(19) United States
(12) Patent Application Publication
(10) Pub. No.: US 2026/0070922 A1
(43) Pub. Date: Mar. 12, 2026

(54) PYRAZOLE AND IMIDAZOLE DERIVATIVES AS DUAL OREXIN AND KAPPA-OPIOID RECEPTORS MODULATORS COMPOSITION, METHODS FOR TREATING NEUROLOGICAL AND PSYCHIATRIC DISORDERS

(71) Applicant: HAGER BIOSCIENCES, LLC, Bethlehem, PA (US)

(72) Inventors: Belew MEKONNEN, Bethlehem, PA (US); Hemantbhai PATEL, Bethlehem, PA (US)

(73) Assignee: HAGER BIOSCIENCES, LLC, Bethlehem, PA (US)

(21) Appl. No.: 19/105,864

(22) PCT Filed: Aug. 31, 2023
(86) PCT No.: PCT/US2023/031782

§ 371 Date: Feb. 24, 2025

Related U.S. Application Data
(60) Provisional application No. 63/403,019, filed on Sep. 1, 2022.

(51) Int. Cl. C07D 498/04; A61K 31/437; A61K 31/444; A61K 31/4545; A61K 31/497; A61K 31/4985; A61K 31/506; A61K 31/519; A61K 31/5365; A61K 31/5377; A61K 31/5383; C07B 59/00; C07D 471/04; C07D 487/04; C07D 519/00

(52) U.S. Cl. CPC C07D 498/04; A61K 31/437; A61K 31/444; A61K 31/4545; A61K 31/497; A61K 31/4985; A61K 31/506; A61K 31/519; A61K 31/5365; A61K 31/5377; A61K 31/5383; C07B 59/002; C07D 471/04; C07D 487/04; C07D 519/00

(57) ABSTRACT

The present disclosure is directed to substituted Pyrazole and Imidazole derivatives of compounds that are antagonists and/or modulators of orexin and kappa-opioid receptors, and which are useful in the treatment or prevention of neurological, psychiatric, cardiovascular and cancer disorders and diseases in which orexin and kappa-opioid receptors are involved or implicated. The disclosure is also directed to pharmaceutical compositions comprising these compounds and the use of these compounds and compositions in the prevention or treatment of such diseases in which orexin and kappa-opioid receptors are involved.

### RELATED APPLICATIONS

This application claims priority to U.S. Ser. No. 63/403,019 filed on Sep. 1, 2022, which is hereby incorporated into this application in its entirety.

### STATEMENT REGARDING FEDERAL SUPPORT

This disclosure was made with partial government support under Federal Award UF1DA054817 awarded by The National Institutes of Drug Abuse of NIH. The government has certain rights in the disclosure.

### FIELD OF THE DISCLOSURE

This disclosure pertains to and provides compounds, compositions and methods for using dual-acting orexin and kappa-opioid receptor antagonists and/or modulators to treat or ameliorate human and animal diseases as therapeutic agents. In particular, any pathological disorder in which both types of orexin and kappa-opioid receptors are pharmacologically involved or implicated. These important therapeutic applications include but are not limited to treating central nervous system (CNS) disorders, neurological, cardiovascular diseases including various cancers that involve or are modulated by orexin and/or kappa-opioid receptors including but not limited to disorders that are responsive to orexin and/or kappa-opioid receptor antagonists, e.g., substance addiction and dependence, cognitive impairment, Alzheimer's disease (AD), posttraumatic stress disorder (PTSD), schizophrenia, panic, anxiety, autism, pain and depression.

### BACKGROUND OF THE DISCLOSURE

The orexins (also known as hypocretins) are comprised of two excitatory hypothalamic neuropeptides: orexin A (OX-A; a 33 amino acid peptide) and orexin B (OX-B; a 28 amino acid peptide). They were simultaneously discovered in 1998 by two research groups searching for new signaling molecules, (1) Sakurai and co-workers (who named them orexin-A and -B) (Sakurai, T. et al, Cell 1998, 92, 573) and (2) de Lecea and co-workers (who named them hypocretin 1 and 2, respectively) (de Lecea, L. et al, Proc. Natl. Acad. Sci. U.S.A. 1998, 95, 322.). These neuropeptides are endogenous ligands for two G protein-coupled receptors (GPCR) named OX₁R and OX₂R (also referred to as Hcrt1 and Hcrt2, respectively) and are derived proteolytically from the same precursor peptide called pre-pro-orexin polypeptide (Sakurai T., et al. The Journal of biological chemistry. 1999; 274, 17771-17776). Though structurally related, the binding affinities of these endogenous ligands for the two GPCRs differ. Orexin A binds to OX₁R with about 100-fold higher affinity than Orexin B, whilst both Orexin A and Orexin B bind to OX₂R with the same affinity (Kodadek, T.; Cai, D. Mol. BioSyst., 2010, 6, 1366-1375). Soon after the discovery of orexin, modulation of the orexin signaling was originally considered for potential novel treatments of narcoleptic or insomniac patients since the role of orexin in regulation of sleep and wakefulness was well-studied and understood, and the discovery of small-molecule modulators of orexin signaling facilitated the development of this class of compounds. Narcoleptic patients show a diminished activity in hypothalamic orexin neurons thereby lowering the amounts of circulating orexin in the cerebrospinal fluid. In contrast, activation of orexin neurons maintains wakefulness and arousal. The effects of orexin signaling on feeding and energy homeostasis were also established earlier and found to be coordinated to the sleep-wake cycle (Kodadek, T.; Cai, D. Mol. BioSyst., 2010, 6, 1366-1375). More recent studies have established the role of orexin and/or kapp-opioid signaling in other key physiological pathways such as neuroendocrine functions (Inutsuka, A.; Yamanaka, A. Front. Endocrinol. 2013, 4:18. doi: 10.3389/fendo.2013.00018), glucose metabolism (Tsuneki, H., et al., Endocrinology, 2016, 157, 4146-4157), stress-adaptive responses (Xiao, F., et al. Neuropharmacology, 2013, 67, 16-24), and addiction/reward-seeking (Aston-Jones, G., et al. Brain Res., 2010, 1314, 74-90). Small molecule orexin antagonists have been broadly categorized into three classes based on their overall receptor selectivity profiles: (1) DORA (dual-acting, or non-selective OX₁R/OX₂R antagonists), (2) SORA-1 (selective OX₁R antagonists), and (3) SORA-2 (selective OX₂R antagonists). It has been shown that while OX₂R knockout mice and OX₁R/OX₂R double knockout mice both show a narcoleptic phenotype, the effect is very muted in OX₁R knockouts (Wang C., et al. Neurosci., 2018, 11, 220. doi: 10.3389/fnmol.2018.00220). Additionally, both DORA and SORA-2 compounds inhibit wakefulness, but SORA-1 compounds do not—thus suggesting that narcoleptic effects are mediated through OX₂R or a combination of OX₁R and OX₂R, but not through OX₁R alone. Thus, it is clear that the discovery and development of differentiated orexin antagonists is crucial to the advancement of this field but most importantly to the development of therapeutic agents for dysregulated biological processes that involve the orexin receptor; especially for non-sleep related indications such as substance addiction, anxiety, panic and PTSD.

Kappa-Opioid Receptor (κOR): Opioid research has been the focus of numerous drug development efforts for CNS or neuropsychiatric disorders thru the modulation of endogenous opioid systems such as the Mu, Kappa, and Delta opioid receptors (MORs, κORs, DORs) and their endogenous ligand peptides; β-endorphins, dynorphins, and enkephalins, respectively. More specific (i.e., preferably), κOR antagonists have shown potential utility in the treatment of stress-related mood-disorders including depression, anxiety, and drug-abuse. Stressful stimuli, especially those occurring during the withdrawal phase, lead to activation of κOR by the endogenous ligand dynorphin. This stress-induced depressive disorder contributes to relapse of drug-seeking behavior, which has been shown to be mitigated by κOR antagonists. In contrast, stimulation of κOR either by its endogenous ligand dynorphin or synthetic agonists such as opioids is known to show anti-nociceptive and analgesic effects.

The simultaneous modulation of different molecular targets or receptors that are co-implicated in a common disease pathway or disorder, using a single molecular entity, could provide better overall efficacy compared to dosing with either a cocktail of medications or a single pill Formulated with multi-component agents. Potential advantages of dual target modulation approach over drug combinations or cocktails include but are not limited to: improved dosing regimen and compliance, reduced drug-drug interactions, simplified and predictable PK/PD relationships and one-dimensional DMPK and safety profiles, potentially synergistic efficacy, reduced potential for tolerance, and reduced regulatory hurdles. This Network Pharmacology approach could provide superior efficacy versus single-target agents to treat multifactorial and multi-etiological disease states such as CNS and/or neurological disorders, cancer, metabolic syndrome, cardiovascular disorders, and more specifically (i.e., preferred) conditions such as addiction disorders. Therefore, a measured but balanced simultaneous modulation of orexin and kappa-opioid receptors (OXR and κOR) (e.g., antagonist of OXR and partial antagonist or inverse agonist or agonist of κOR) may provide unique, first in class and high efficacy new molecular entities as a therapeutic agent(s) for numerous dysregulated biological processes that involve orexin and/or kappa-opioid receptors. The compounds, compositions and methods provide solutions to such problems in the art.

### SUMMARY OF THE DISCLOSURE

This disclosure addresses the aforementioned therapeutic needs by providing compounds of Formula I or II, in different embodiments:

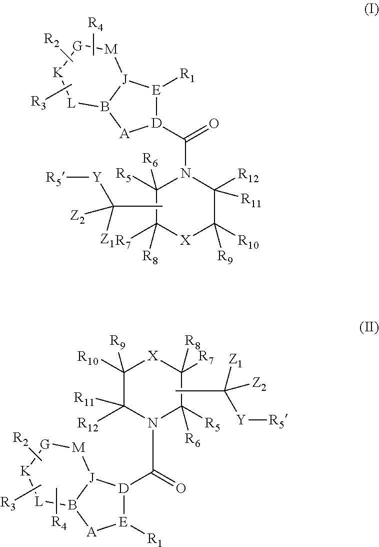

wherein the variables are as defined herein, including any pharmaceutically acceptable salts, solvates, adducts, polymorphs, and isomers thereof. Compounds of Formulas I or II, respectively, can be used to treat the conditions described herein, such as through modulations of orexin and/or kappa-opioid receptors. In some embodiments, the compounds provide a balanced simultaneous modulation of orexin and kappa-opioid receptors (OXR and κOR) (e.g., antagonist of OXR and partial antagonist or inverse agonist or agonist of κOR).

The present disclosure also provides compositions that comprise the above compounds or a pharmaceutically acceptable salt thereof. In another aspect of the disclosure, there is provided a method for treating CNS disorders such as, among others, substance addiction and dependence, posttraumatic stress disorder (PTSD), schizophrenia, panic, anxiety, pain, depression, cognitive impairment and Alzheimer's disease (AD) in a subject in need or at risk thereof, the method comprising the step of administering to said subject a therapeutically effective amount of orexin and/or kappa-opioid receptor antagonists and/or modulators or a pharmaceutically acceptable salt thereof. In certain embodiments of the disclosure, the antagonists and/or modulators or a pharmaceutically acceptable salt thereof could be Formulated to be administered periodically, for example every 3, 6 to 24 hours or weekly as deemed clinically beneficial.

### DETAILED DESCRIPTION OF THE DISCLOSURE

The present disclosure pertains to a fused 6 and 5 membered ring system derivatives of Formula (I) and/or (II) wherein the fused 6 and 5 membered rings are as described in the full disclosure enabling structural descriptions, to pharmaceutically acceptable salts thereof, to their preparation, to pharmaceutical compositions containing one or more compounds of Formula (I) and/or (II), and to their use as pharmaceuticals and therapeutic agents, particularly to their use as orexin and/or kappa-opioid receptor antagonists and/or modulators. These novel agents as described by Formula (I) and/or (II) which are non-peptide modulators of human orexin and/or kappa-opioid receptors and are potentially useful in the treatment of disorders relating to orexinergic and kappa-opioid receptors dysfunctions; including but not limited for such disorders like substance addiction, anxiety, panic, cognitive dysfunctions, mood, or appetite, sleep, Alzheimer (AD), metabolic syndrome, and hypertension; and especially these compounds could be of great therapeutic value in the treatment of anxiety disorders, pain, addiction disorders, and sleep disorders.

A first aspect of the disclosure relates to and provides compounds of the Formula (I) or (II), respectively:

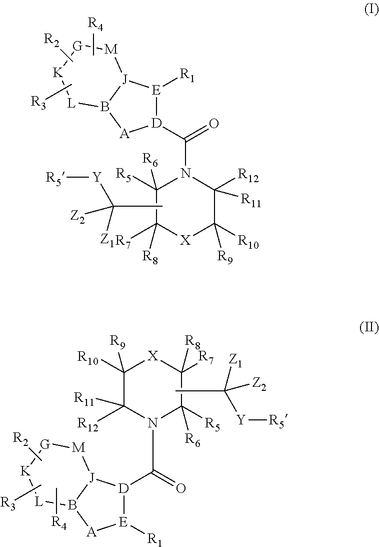

wherein:
R₁, includes E in which is Carbon (C) but not Nitrogen (N), and E is linked to J or D with a double bond, and R₁ is selected from the group consisting of H, alkyl, alkoxy, cycloalkyl, phenyl, aromatic or aryl, heteroaryl (5-6 membered ring), substituted aromatic or aryl, substituted heteroaryl (5-6 membered ring); or, when R₁ is heteroaryl, R₁ is preferably a 5 or 6-membered heteroaryl selected from the group consisting of pyrazolyl, triazolyl, oxazolyl, thiazolyl, thiophenyl, pyridinyl, pyrimidinyl, pyrazinyl, and pyridazinyl; wherein said aromatic, aryl or heteroaryl is unsubstituted, mono-, or di-substituted, wherein the substituents are independently selected from the group consisting of (C₁₋₄)alkyl, (C₁₋₄)alkoxy, halogen, (C₁₋₃)fluoroalkyl, (C₁₋₃)fluoroalkoxy and (C₃₋₇)cycloalkyl;
R₂, R₃, and R₄ are independently selected from the group consisting of H, halogen (such as F, Cl, Br), alkyl, substituted alkyl, (C₁₋₄)alkyl, (C₄)alkoxy, (C₁₋₃)fluoroalkyl, (C₁₋₃)fluoroalkoxy and (C₃₋₇)cycloalkyl; wherein each of R₂, R₃ and R₄ is independently and optionally substituted at each substitutable position with up to three (3) substituents independently selected from one, two, or all R₂, R₃ and R₄;
R₅═H, F, CH₃, alkyl, substituted alkyl, (C₁₋₃)fluoroalkyl, cycloalkyl, R₅ replaced with the carbon bearing Y, Z₁ and Z₂, wherein R_{5′} is defined herein;
R_{5′}=aromatic or aryl, heteroaryl (5-6 membered ring), substituted aromatic or aryl, substituted heteroaryl or fused two heteroaryl ring systems (5-6 membered ring); wherein said aromatic, aryl or heteroaryl is unsubstituted, mono-, or di-substituted or tri-substituted, wherein the substituents are independently selected from the group consisting of (C₁₋₄)alkyl, (C₁₋₄)alkoxy, halogen, (C₁₋₃)fluoroalkyl, (C₁₋₃)fluoroalkoxy, (C₃₋₇)cycloalkyl, (C₃₋₇)heterocycloalkyl;
R₆═H, F, CH₃, alkyl, substituted alkyl, (C₁₋₃)fluoroalkyl, cycloalkyl, R₆ connected to either R₁₀ or R₁₁ as alkyl to form a (C₁₋₃)alkyl bridge cyclic structure;
R₇═H, F, CH₃, alkyl, substituted alkyl, (C₁₋₃)fluoroalkyl, cycloalkyl, R₇ replaced with the carbon bearing Y, Z1 and Z2, wherein R_{5′} is defined herein;
R₈═H, F, CH₃, alkyl, substituted alkyl, (C₁₋₃)fluoroalkyl, cycloalkyl, R₈ connected to either R₁₀, R₁₁ or R₁₂ as alkyl to form a (C₁₋₃)alkyl bridge cyclic structure;
R₉═H, F, CH₃, alkyl, substituted alkyl, (C₁₋₃)fluoroalkyl, cycloalkyl, R₉ connected to either R₆ or R₁₂ as alkyl to form a (C₁₋₃)alkyl bridge cyclic structure;
R₁₀═H, F, CH₃, alkyl, substituted alkyl, (C₁₋₃)fluoroalkyl, cycloalkyl, or R₁₀ connected to R₆ or R₁₁ as alkyl to form a (C₁₋₃)alkyl bridge cyclic structure;
R₁₁═H, F, CH₃, alkyl, substituted alkyl, (C₁₋₃)fluoroalkyl, cycloalkyl, or R₁₁ connected to R₆ as alkyl to form a (C₁₋₃)alkyl bridge cyclic structure;
R₁₂═H, F, CH₃, alkyl, substituted alkyl, (C₁₋₃)fluoroalkyl, cycloalkyl, or R₁₂ connected to R₉ as alkyl to form a (C₁₋₃)alkyl bridge cyclic structure;
X═CH₂, O, nothing (for example to provide five membered pyrrolidine ring), CRₐR_{b} (where Rₐ and R_{b}=alkyl, cycloalkyl, Fluoroalkyl); wherein the carbon atom at position 2 of the piperidine or pyrrolidine is preferred in absolute (S)-configuration; in contrast, the carbon atom at position 2 of the of the morpholine ring (when X═O, oxygen) is preferred in absolute (R)-configuration;
Y═O, NH, nothing (R₅′ attached directly to the carbon bearing Z₁ and Z₂ group), CH₂OR_{5′}, CH₂, NRₐ (wherein Rₐ=alkyl, cycloalkyl, heteroalkyl), or Y could be chosen as a preferred linker as a 5 or 6-membered heteroaryl selected from the group consisting of pyrrolyl, pyrazolyl, triazolyl, oxazolyl, thiazolyl, oxadiazolyl; and,
Z₁, Z₂═H, F, (C₁₋₄)alkyl, (C₁₋₃)fluoroalkyl, (C₁₋₃)fluoroalkoxy, (C₂₋₇)cycloalkyl or independently chosen from one of these groups;
also wherein:
fused or unfused ring system A-B-J-D-E is a five-membered heteroaryl, for example, imidazole (wherein A, J=Nitrogen while B, E, D=Carbon), pyrazole (when A, B=Nitrogen, while D, E, J=Carbon) fused or unfused with an additional ring systems;
fused ring system B-J-M-G-K-L is an arrangement of these outlined variables to provide groups consisting of a 6-membered aromatic, 6-membered aryl, 6-membered substituted aromatic, 6-membered substituted aryl, 6-membered substituted heteroaryl, 6-membered unsubstituted heteroaryl, 5 or 6-membered cycloalkyl, and 5 or 6-membered heterocycloalkyl;
wherein the preferred groups are:
A=Nitrogen, such as in Imidazole or Pyrazole ring systems;
B=Carbon or Nitrogen
J=Carbon or Nitrogen
D=Carbon
E=Carbon, wherein R₁ is as defined above;
M=Carbon, CH, CHR₂, CHR₃, CR₂R₃, CR₂, CR₃, CR₄, O, N;
G=Carbon, CH, CHR₂, CHR₃, CR₂R₃, CR₂, CR₃, CR₄, O;
K=Carbon, CH, CHR₂, CHR₃, CR₂R₃, CR₂, CR₃, CR₄, O; and,
L=Carbon, CH, CHR₂, CHR₃, CR₂R₃, CR₂, CR₃, CR₄, O, and N.

In a more specific (i.e., preferred) embodiment, the disclosure provides a compound that has the Formula I-a and II-a, wherein the ring system (illustrated by A-B-J-D-E variables in Formula (I) or (II)) fused to the 6-membered ring is preferred as an imidazolo ring system as represented by embodiment Formula I-a or II-a, respectively, herein:

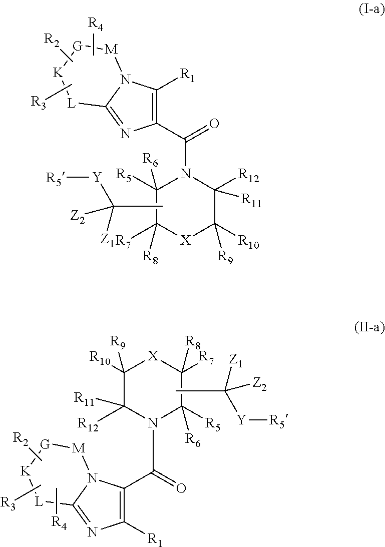

wherein:
R₁ is selected from the group consisting of H, alkyl, alkoxy, cycloalkyl, phenyl, aromatic or aryl, heteroaryl (5-6 membered ring), substituted aromatic or aryl, substituted heteroaryl (5-6 membered ring); or, when R₁ is heteroaryl, R₁ is preferably a 5 or 6-membered heteroaryl selected from the group consisting of pyrazolyl, triazolyl, oxazolyl, thiazolyl, thiophenyl, pyridinyl, pyrimidinyl, pyrazinyl, and pyridazinyl; wherein said aromatic, aryl or heteroaryl is unsubstituted, mono-, or di-substituted, wherein the substituents are independently selected from the group consisting of (C₁₋₄)alkyl, (C₁₋₄)alkoxy, halogen, (C₁₋₃)fluoroalkyl, (C₁₋₃)fluoroalkoxy and (C₃₋₇)cycloalkyl;
R₂, R₃, and R₄ are independently selected from the group consisting of H, halogen (such as F, Cl, Br), alkyl, substituted alkyl, (C₁₋₄)alkyl, (C₁₋₄)alkoxy, (C₁₋₃)fluoroalkyl, (C₁₋₃)fluoroalkoxy and (C₃ 7)cycloalkyl; wherein each of R₂, R₃ and R₄ is independently and optionally substituted at each substitutable position with up to three (3) substituents independently selected from one, two, or all R₂, R₃ and R₄;
R₅═H, F, CH₃, alkyl, substituted alkyl, (C₁₋₃)fluoroalkyl, cycloalkyl, R₅ replaced with the carbon bearing Y, Z1 and Z2, wherein R_{5′} is defined herein;
R_{5′}=aromatic or aryl, heteroaryl (5-6 membered ring), substituted aromatic or aryl, substituted heteroaryl or fused two heteroaryl ring systems (5-6 membered ring); wherein said aromatic, aryl or heteroaryl is unsubstituted, mono-, or di-substituted or tri-substituted, wherein the substituents are independently selected from the group consisting of (C₁₋₄)alkyl, (C₁₋₄)alkoxy, halogen, (C₁₋₃)fluoroalkyl, (C₁₋₃)fluoroalkoxy, (C₃₋₇)cycloalkyl, (C₃₋₇)heterocycloalkyl;
R₆═H, F, CH₃, alkyl, substituted alkyl, (C₁₋₃)fluoroalkyl, cycloalkyl, R₆ connected to either R₁₀ or R₁₁ as alkyl to form a (C₁₋₃)alkyl bridge cyclic structure;
R₇═H, F, CH₃, alkyl, substituted alkyl, (C₁₋₃)fluoroalkyl, cycloalkyl, R₇ replaced with the carbon bearing Y, Z₁ and Z₂, wherein R_{5′} is defined herein;
R₈═H, F, CH₃, alkyl, substituted alkyl, (C₁₋₃)fluoroalkyl, cycloalkyl, R₈ connected to either R₁₀, R₁₁ or R₁₂ as alkyl to form a (C₁₋₃)alkyl bridge cyclic structure;
R₉═H, F, CH₃, alkyl, substituted alkyl, (C₁₋₃)fluoroalkyl, cycloalkyl, R₉ connected to either R₆ or R₁₂ as alkyl to form a (C₁₋₃)alkyl bridge cyclic structure;
R₁₀═H, F, CH₃, alkyl, substituted alkyl, (C₁₋₃)fluoroalkyl, cycloalkyl, or R₁₀ connected to R₆ or R₁₁ as alkyl to form a (C₁₋₃)alkyl bridge cyclic structure;
R₁₁═H, F, CH₃, alkyl, substituted alkyl, (C₁₋₃)fluoroalkyl, cycloalkyl, or R₁₁ connected to R₆ as alkyl to form a (C₁₋₃)alkyl bridge cyclic structure;
R₁₂═H, F, CH₃, alkyl, substituted alkyl, (C₁₋₃)fluoroalkyl, cycloalkyl, or R₁₂ connected to R₉ as alkyl to form a (C₁₋₃)alkyl bridge cyclic structure;
X═CH₂, O, nothing (for example to provide five membered pyrrolidine ring), CRₐR_{b} (where Rₐ and R_{b}=alkyl, cycloalkyl, Fluoroalkyl); wherein the carbon atom at position 2 of the piperidine or pyrrolidine is preferred in absolute (S)-configuration; in contrast, the carbon atom at position 2 of the of the morpholine ring (when X═O, oxygen) is preferred in absolute (R)-configuration;
Y═O, NH, nothing (R₅′ attached directly to the carbon bearing Z₁ and Z₂ group), CH₂OR_{5′}, CH₂, NRₐ (wherein Rₐ=alkyl, cycloalkyl, heteroalkyl), or Y could be chosen as a preferred linker as a 5 or 6-membered heteroaryl selected from the group consisting of pyrrolyl, pyrazolyl, triazolyl, oxazolyl, thiazolyl, oxadiazolyl; and,
Z₁, Z₂═H, F, (C₁₋₄)alkyl, (C₁₋₃)fluoroalkyl, (C₁₋₃)fluoroalkoxy, (C₂₋₇)cycloalkyl or independently chosen from one of these groups;
also wherein:
fused ring system B-J-M-G-K-L is an arrangement of these outlined variables to provide groups consisting of a 6-membered aromatic, 6-membered aryl, 6-membered substituted aromatic, 6-membered substituted aryl, 6-membered substituted heteroaryl, 6-membered unsubstituted heteroaryl, 5 or 6-membered cycloalkyl, and 5 or 6-membered heterocycloalkyl; wherein the preferred groups are:
M=Carbon, CH, CHR₂, CHR₃, CR₂R₃, CR₂, CR₃, CR₄, O, N;
G=Carbon, CH, CHR₂, CHR₃, CR₂R₃, CR₂, CR₃, CR₄, O;
K=Carbon, CH, CHR₂, CHR₃, CR₂R₃, CR₂, CR₃, CR₄, O; and,
L=Carbon, CH, CHR₂, CHR₃, CR₂R₃, CR₂, CR₃, CR₄, O, and N.

In a more specific (i.e., preferred) embodiment, the disclosure provides a compound that has the Formula I-b and II-b, wherein the ring system (illustrated by A-B-J-D-E variables in Formula (I) or (II)) fused to the 6-membered ring is preferred as a pyrazolo ring system as represented by embodiment Formula I-b or II-b, respectively, herein:

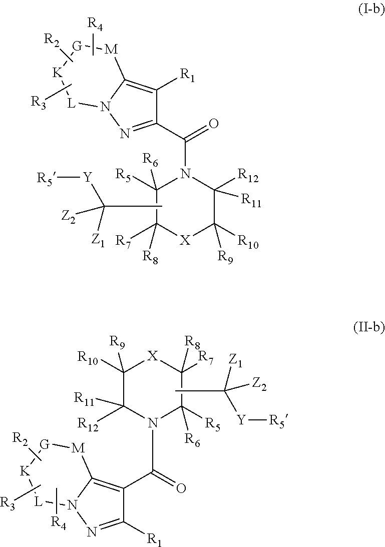

wherein:
R₁ is selected from the group consisting of H, alkyl, alkoxy, cycloalkyl, phenyl, aromatic or aryl, heteroaryl (5-6 membered ring), substituted aromatic or aryl, substituted heteroaryl (5-6 membered ring); or, when R₁ is heteroaryl, R₁ is preferably a 5 or 6-membered heteroaryl selected from the group consisting of pyrazolyl, triazolyl, oxazolyl, thiazolyl, thiophenyl, pyridinyl, pyrimidinyl, pyrazinyl, and pyridazinyl; wherein said aromatic, aryl or heteroaryl is unsubstituted, mono-, or di-substituted, wherein the substituents are independently selected from the group consisting of (C₁₋₄)alkyl, (C₁₋₄)alkoxy, halogen, (C₁₋₃)fluoroalkyl, (C₁₋₃)fluoroalkoxy and (C₃₋₇)cycloalkyl;
R₂, R₃, and R₄ are independently selected from the group consisting of H, halogen (such as F, Cl, Br), alkyl, substituted alkyl, (C₁₋₄)alkyl, (C₁₋₄)alkoxy, (C₁₋₃)fluoroalkyl, (C₁₋₃)fluoroalkoxy and (C₃₋₇)cycloalkyl; wherein each of R₂, R₃ and R₄ is independently and optionally substituted at each substitutable position with up to three (3) substituents independently selected from one, two, or all R₂, R₃ and R₄;
R₅═H, F, CH₃, alkyl, substituted alkyl, (C₁₋₃)fluoroalkyl, cycloalkyl, R₅ replaced with the carbon bearing Y, Z₁ and Z₂, wherein R₅′ is defined herein;
R_{5′}=aromatic or aryl, heteroaryl (5-6 membered ring), substituted aromatic or aryl, substituted heteroaryl or fused two heteroaryl ring systems (5-6 membered ring); wherein said aromatic, aryl or heteroaryl is unsubstituted, mono-, or di-substituted or tri-substituted, wherein the substituents are independently selected from the group consisting of (C₁₋₄)alkyl, (C₁₋₄)alkoxy, halogen, (C₁₋₃)fluoroalkyl, (C₁₋₃)fluoroalkoxy, (C₃₋₇)cycloalkyl, (C₃₋₇)heterocycloalkyl;
R₆═H, F, CH₃, alkyl, substituted alkyl, (C₁₋₃)fluoroalkyl, cycloalkyl, R₆ connected to either R₁₀ or R₁₁ as alkyl to form a (C₁₋₃)alkyl bridge cyclic structure;
R₇═H, F, CH₃, alkyl, substituted alkyl, (C₁₋₃)fluoroalkyl, cycloalkyl, R₇ replaced with the carbon bearing Y, Z₁ and Z₂, wherein R₅ is defined herein;
R₆═H, F, CH₃, alkyl, substituted alkyl, (C₁₋₃)fluoroalkyl, cycloalkyl, R₈ connected to either R₁₀, R₁₁ or R₁₂ as alkyl to form a (C₁₋₃)alkyl bridge cyclic structure;
R₉═H, F, CH₃, alkyl, substituted alkyl, (C₁₋₃)fluoroalkyl, cycloalkyl, R₉ connected to either R₆ or R₁₂ as alkyl to form a (C₁₋₃)alkyl bridge cyclic structure;
R₁₀═H, F, CH₃, alkyl, substituted alkyl, (C₁₋₃)fluoroalkyl, cycloalkyl, or R₁₀ connected to R₆ or R₁₁ as alkyl to form a (C₁₋₃)alkyl bridge cyclic structure;
R₁₁═H, F, CH₃, alkyl, substituted alkyl, (C₁₋₃)fluoroalkyl, cycloalkyl, or R₁₁ connected to R₆ as alkyl to form a (C₁₋₃)alkyl bridge cyclic structure;
R₁₂═H, F, CH₃, alkyl, substituted alkyl, (C₁₋₃)fluoroalkyl, cycloalkyl, or R₁₂ connected to R₉ as alkyl to form a (C₁₋₃)alkyl bridge cyclic structure;
X═CH₂, O, nothing (for example to provide five membered pyrrolidine ring), CRₐR_{b} (where Rₐ and R_{b}=alkyl, cycloalkyl, Fluoroalkyl); wherein the carbon atom at position 2 of the piperidine or pyrrolidine is preferred in absolute (S)-configuration; in contrast, the carbon atom at position 2 of the of the morpholine ring (when X═O, oxygen) is preferred in absolute (R)-configuration;
Y═O, NH, nothing (R₅′ attached directly to the carbon bearing Z₁ and Z₂ group), CH₂OR₅′, CH₂, NRₐ (wherein Rₐ=alkyl, cycloalkyl, heteroalkyl), or Y could be chosen as a preferred linker as a 5 or 6-membered heteroaryl selected from the group consisting of pyrrolyl, pyrazolyl, triazolyl, oxazolyl, thiazolyl, oxadiazolyl; and,
Z₁, Z₂═H, F, (C₁₋₄)alkyl, (C₁₋₃)fluoroalkyl, (C₁₋₃)fluoroalkoxy, (C₂₋₇)cycloalkyl or independently chosen from one of these groups;
also wherein:
fused ring system B-J-M-G-K-L is an arrangement of these outlined variables to provide groups consisting of a 6-membered aromatic, 6-membered aryl, 6-membered substituted aromatic, 6-membered substituted aryl, 6-membered substituted heteroaryl, 6-membered unsubstituted heteroaryl, 5 or 6-membered cycloalkyl, and 5 or 6-membered heterocycloalkyl;
wherein the preferred groups are:
M=Carbon, CH, CHR₂, CHR₃, CR₂R₃, CR₂, CR₃, CR₄, O, N;
G=Carbon, CH, CHR₂, CHR₃, CR₂R₃, CR₂, CR₃, CR₄, O;
K=Carbon, CH, CHR₂, CHR₃, CR₂R₃, CR₂, CR₃, CR₄, O; and,
L=Carbon, CH, CHR₂, CHR₃, CR₂R₃, CR₂, CR₃, CR₄, O, and N.

A further embodiment of the disclosure relates to preferred compounds wherein the imidazole fused six-membered ring is preferred as shown herein according to embodiment Formula I-a1 or II-a1, respectively:

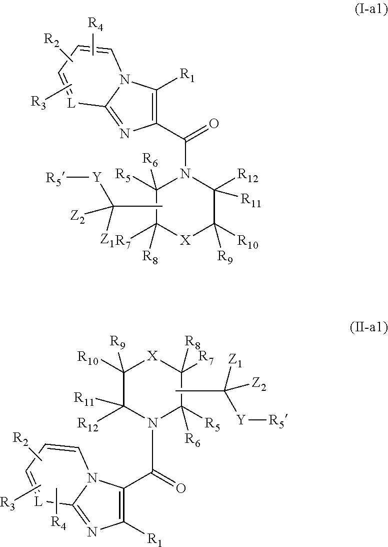

wherein:
R₁ is selected from the group consisting of H, alkyl, alkoxy, cycloalkyl, phenyl, aromatic or aryl, heteroaryl (5-6 membered ring), substituted aromatic or aryl, substituted heteroaryl (5-6 membered ring); or, when R₁ is heteroaryl, R₁ is preferably a 5 or 6-membered heteroaryl selected from the group consisting of pyrazolyl, triazolyl, oxazolyl, thiazolyl, thiophenyl, pyridinyl, pyrimidinyl, pyrazinyl, and pyridazinyl; wherein said aromatic, aryl or heteroaryl is unsubstituted, mono-, or di-substituted, wherein the substituents are independently selected from the group consisting of (C₁₋₄)alkyl, (C₁₋₄)alkoxy, halogen, (C₁₋₃)fluoroalkyl, (C₁₋₃)fluoroalkoxy and (C₃₋₇)cycloalkyl;
R₂, R₃, and R₄ are independently selected from the group consisting of H, halogen (such as F, Cl, Br), alkyl, substituted alkyl, (C₁₋₄)alkyl, (C₁₋₄)alkoxy, (C₁₋₃)fluoroalkyl, (C₁₋₃)fluoroalkoxy and (C₃₋₇)cycloalkyl; wherein each of R₂, R₃ and R₄ is independently and optionally substituted at each substitutable position with up to three (3) substituents independently selected from one, two, or all R₂, R₃ and R₄;
R₅═H, F, CH₃, alkyl, substituted alkyl, (C₁₋₃)fluoroalkyl, cycloalkyl, R₅ replaced with the carbon bearing Y, Z₁ and Z₂, wherein R₅ is defined herein;
R_{5′}=aromatic or aryl, heteroaryl (5-6 membered ring), substituted aromatic or aryl, substituted heteroaryl or fused two heteroaryl ring systems (5-6 membered ring); wherein said aromatic, aryl or heteroaryl is unsubstituted, mono-, or di-substituted or tri-substituted, wherein the substituents are independently selected from the group consisting of (C₁₋₄)alkyl, (C₁₋₄)alkoxy, halogen, (C₁₋₃)fluoroalkyl, (C₁₋₃)fluoroalkoxy, (C₃₋₇)cycloalkyl, (C₃₋₇)heterocycloalkyl;
R₆═H, F, CH₃, alkyl, substituted alkyl, (C₁₋₃)fluoroalkyl, cycloalkyl, R₆ connected to either R₁₀ or R₁₁ as alkyl to form a (C₁₋₃)alkyl bridge cyclic structure;
R₇═H, F, CH₃, alkyl, substituted alkyl, (C₁₋₃)fluoroalkyl, cycloalkyl, R₇ replaced with the carbon bearing Y, Z₁ and Z₂, wherein R_{5′} is defined herein;
R₈═H, F, CH₃, alkyl, substituted alkyl, (C₁₋₃)fluoroalkyl, cycloalkyl, R₈ connected to either R₁₀, R₁₁ or R₁₂ as alkyl to form a (C₁₋₃)alkyl bridge cyclic structure;
R₉═H, F, CH₃, alkyl, substituted alkyl, (C₁₋₃)fluoroalkyl, cycloalkyl, R₉ connected to either R₆ or R₁₂ as alkyl to form a (C₁₋₃)alkyl bridge cyclic structure;
R₁₀═H, F, CH₃, alkyl, substituted alkyl, (C₁₋₃)fluoroalkyl, cycloalkyl, or R₁₀ connected to R₆ or R₁₁ as alkyl to form a (C₁₋₃)alkyl bridge cyclic structure;
R₁₁═H, F, CH₃, alkyl, substituted alkyl, (C₁₋₃)fluoroalkyl, cycloalkyl, or R₁₁ connected to R₆ as alkyl to form a (C₁₋₃)alkyl bridge cyclic structure;
R₁₂═H, F, CH₃, alkyl, substituted alkyl, (C₁₋₃)fluoroalkyl, cycloalkyl, or R₁₂ connected to R₉ as alkyl to form a (C₁₋₃)alkyl bridge cyclic structure;
X═CH₂, O, nothing (for example to provide five membered pyrrolidine ring), CRₐR_{b} (where Rₐ and R_{b}=alkyl, cycloalkyl, fluoroalkyl); wherein the carbon atom at position 2 of the piperidine or pyrrolidine is preferred in absolute (S)-configuration; in contrast, the carbon atom at position 2 of the of the morpholine ring (when X═O, oxygen) is preferred in absolute (R)-configuration;
Y═O, NH, nothing (R₅′ attached directly to the carbon bearing Z₁ and Z₂ group), CH₂OR₅′, CH₂, NRₐ (wherein Rₐ=alkyl, cycloalkyl, heteroalkyl), or Y could be chosen as a preferred linker as a 5 or 6-membered heteroaryl selected from the group consisting of pyrrolyl, pyrazolyl, triazolyl, oxazolyl, thiazolyl, oxadiazolyl; and, Z₁, Z₂═H, F, (C₁₋₄)alkyl, (C₁₋₃)fluoroalkyl, (C₁₋₃)fluoroalkoxy, (C₂₋₇)cycloalkyl or independently chosen from one of these groups;
wherein the preferred groups are:
L=Carbon, CH, CHR₂, CHR₃, CR₂R₃, CR₂, CR₃, CR₄, and N.

A further embodiment of the disclosure relates to compounds wherein the imidazole fused six-membered ring is preferred as shown herein according to embodiment Formula I-a2 or II-a2, respectively:

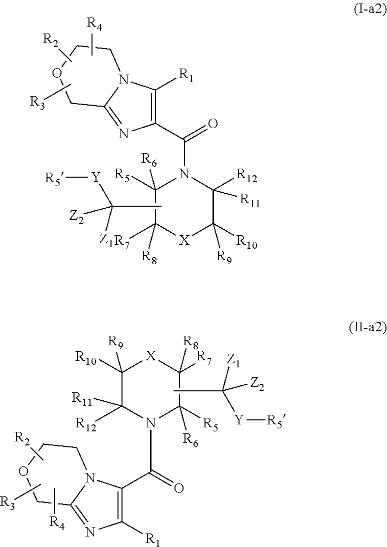

wherein:
R₁ is selected from the group consisting of H, alkyl, alkoxy, cycloalkyl, phenyl, aromatic or aryl, heteroaryl (5-6 membered ring), substituted aromatic or aryl, substituted heteroaryl (5-6 membered ring); or, when R₁ is heteroaryl, R₁ is preferably a 5 or 6-membered heteroaryl selected from the group consisting of pyrazolyl, triazolyl, oxazolyl, thiazolyl, thiophenyl, pyridinyl, pyrimidinyl, pyrazinyl, and pyridazinyl; wherein said aromatic, aryl or heteroaryl is unsubstituted, mono-, or di-substituted, wherein the substituents are independently selected from the group consisting of (C₁₋₄)alkyl, (C₁₋₄)alkoxy, halogen, (C₁₋₃)fluoroalkyl, (C₁₋₃)fluoroalkoxy and (C₃₋₇)cycloalkyl;
R₂, R₃, and R₄ are independently selected from the group consisting of H, halogen (such as F, Cl, Br), alkyl, substituted alkyl, (C₁₋₄)alkyl, (C₁₋₄)alkoxy, (C₁₋₃)fluoroalkyl, (C₁₋₃)fluoroalkoxy and (C₃₋₇)cycloalkyl; wherein each of R₂, R₃ and R₄ is independently and optionally substituted at each substitutable position with up to three (3) substituents independently selected from one, two, or all R₂, R₃ and R₄;
R₅═H, F, CH₃, alkyl, substituted alkyl, (C₁₋₃)fluoroalkyl, cycloalkyl, R₅ replaced with the carbon bearing Y, Z₁ and Z₂, wherein R₅ is defined herein;
R_{5′}=aromatic or aryl, heteroaryl (5-6 membered ring), substituted aromatic or aryl, substituted heteroaryl or fused two heteroaryl ring systems (5-6 membered ring); wherein said aromatic, aryl or heteroaryl is unsubstituted, mono-, or di-substituted or tri-substituted, wherein the substituents are independently selected from the group consisting of (C₁a)alkyl, (C₁₋₄)alkoxy, halogen, (C₁₋₃)fluoroalkyl, (C₁₋₃)fluoroalkoxy, (C₃₋₇)cycloalkyl, (C₃₋₇)heterocycloalkyl;
R₆═H, F, CH₃, alkyl, substituted alkyl, (C₁₋₃)fluoroalkyl, cycloalkyl, R₆ connected to either R₁₀ or R₁₁ as alkyl to form a (C₁₋₃)alkyl bridge cyclic structure;
R₇═H, F, CH₃, alkyl, substituted alkyl, (C₁₋₃)fluoroalkyl, cycloalkyl, R₇ replaced with the carbon bearing Y, Z₁ and Z₂, wherein R₅ is defined herein;
R₆═H, F, CH₃, alkyl, substituted alkyl, (C₁₋₃)fluoroalkyl, cycloalkyl, R₈ connected to either R₁₀, R₁₁ or R₁₂ as alkyl to form a (C₁₋₃)alkyl bridge cyclic structure;
R₉═H, F, CH₃, alkyl, substituted alkyl, (C₁₋₃)fluoroalkyl, cycloalkyl, R₉ connected to either R₀ or R₁₂ as alkyl to form a (C₁₋₃)alkyl bridge cyclic structure;
R₁₀═H, F, CH₃, alkyl, substituted alkyl, (C₁₋₃)fluoroalkyl, cycloalkyl, or R₁₀ connected to R₆ or R₁₁ as alkyl to form a (C₁₋₃)alkyl bridge cyclic structure;
R₁₁═H, F, CH₃, alkyl, substituted alkyl, (C₁₋₃)fluoroalkyl, cycloalkyl, or R₁₁ connected to R₆ as alkyl to form a (C₁₋₃)alkyl bridge cyclic structure;
R₁₂═H, F, CH₃, alkyl, substituted alkyl, (C₁₋₃)fluoroalkyl, cycloalkyl, or R₁₂ connected to R₉ as alkyl to form a (C₁₋₃)alkyl bridge cyclic structure;
X═CH₂, O, nothing (for example to provide five membered pyrrolidine ring), CRₐR_{b} (where Rₐ and R_{b}=alkyl, cycloalkyl, Fluoroalkyl); wherein the carbon atom at position 2 of the piperidine or pyrrolidine is preferred in absolute (S)-configuration; in contrast, the carbon atom at position 2 of the of the morpholine ring (when X═O, oxygen) is preferred in absolute (R)-configuration;
Y═O, NH, nothing (R₅′ attached directly to the carbon bearing Z₁ and Z₂ group), CH₂OR₅′, CH₂, NRₐ (wherein Rₐ=alkyl, cycloalkyl, heteroalkyl), or Y could be chosen as a preferred linker as a 5 or 6-membered heteroaryl selected from the group consisting of pyrrolyl, pyrazolyl, triazolyl, oxazolyl, thiazolyl, oxadiazolyl; and,
Z₁, Z₂═H, F, (C₁₋₄)alkyl, (C₁₋₃)fluoroalkyl, (C₁₋₃)fluoroalkoxy, (C₂₋₇)cycloalkyl or independently chosen from one of these groups.

A further embodiment of the disclosure relates to preferred compounds wherein the imidazole fused six-membered ring is preferred as shown herein according to embodiment Formula I-a3 or II-a3, respectively:

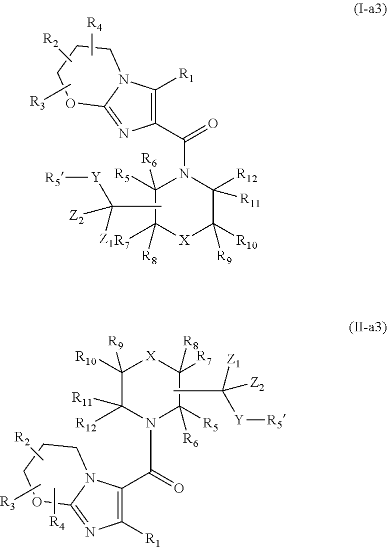

wherein:
R₁ is selected from the group consisting of H, alkyl, alkoxy, cycloalkyl, phenyl, aromatic or aryl, heteroaryl (5-6 membered ring), substituted aromatic or aryl, substituted heteroaryl (5-6 membered ring); or, when R₁ is heteroaryl, R₁ is preferably a 5 or 6-membered heteroaryl selected from the group consisting of pyrazolyl, triazolyl, oxazolyl, thiazolyl, thiophenyl, pyridinyl, pyrimidinyl, pyrazinyl, and pyridazinyl; wherein said aromatic, aryl or heteroaryl is unsubstituted, mono-, or di-substituted, wherein the substituents are independently selected from the group consisting of (C₁₋₄)alkyl, (C₁₋₄)alkoxy, halogen, (C₁₋₃)fluoroalkyl, (C₁₋₃)fluoroalkoxy and (C₃₋₇)cycloalkyl;
R₂, R₃, and R₄ are independently selected from the group consisting of H, halogen (such as F, Cl, Br), alkyl, substituted alkyl, (C₁₋₄)alkyl, (C₁₋₄)alkoxy, (C₁₋₃)fluoroalkyl, (C₁₋₃)fluoroalkoxy and (C₃₋₇)cycloalkyl; wherein each of R₂, R₃ and R₄ is independently and optionally substituted at each substitutable position with up to three (3) substituents independently selected from one, two, or all R₂, R₃ and R₄;
R₅═H, F, CH₃, alkyl, substituted alkyl, (C₁₋₃)fluoroalkyl, cycloalkyl, R₅ replaced with the carbon bearing Y, Z₁ and Z₂, wherein R₅ is defined herein;
R_{5′}=aromatic or aryl, heteroaryl (5-6 membered ring), substituted aromatic or aryl, substituted heteroaryl or fused two heteroaryl ring systems (5-6 membered ring); wherein said aromatic, aryl or heteroaryl is unsubstituted, mono-, or di-substituted or tri-substituted, wherein the substituents are independently selected from the group consisting of (C₁a)alkyl, (C₁₋₄)alkoxy, halogen, (C₁₋₃)fluoroalkyl, (C₁₋₃)fluoroalkoxy, (C₃₋₇)cycloalkyl, (C₃₋₇)heterocycloalkyl;
R₆═H, F, CH₃, alkyl, substituted alkyl, (C₁₋₃)fluoroalkyl, cycloalkyl, R₆ connected to either R₁₀ or R₁₁ as alkyl to form a (C₁₋₃)alkyl bridge cyclic structure;
R₇═H, F, CH₃, alkyl, substituted alkyl, (C₁₋₃)fluoroalkyl, cycloalkyl, R₇ replaced with the carbon bearing Y, Z₁ and Z₂, wherein R₅′ is defined herein;
R₆═H, F, CH₃, alkyl, substituted alkyl, (C₁₋₃)fluoroalkyl, cycloalkyl, R₈ connected to either R₁₀. R₁₁ or R₁₂ as alkyl to form a (C₁₋₃)alkyl bridge cyclic structure;
R₉═H, F, CH₃, alkyl, substituted alkyl, (C₁₋₃)fluoroalkyl, cycloalkyl, R₉ connected to either R₆ or R₁₂ as alkyl to form a (C₁₋₃)alkyl bridge cyclic structure;
R₁₀═H, F, CH₃, alkyl, substituted alkyl, (C₁₋₃)fluoroalkyl, cycloalkyl, or R₁₀ connected to R₆ or R₁₁ as alkyl to form a (C₁₋₃)alkyl bridge cyclic structure;
R₁₁═H, F, CH₃, alkyl, substituted alkyl, (C₁₋₃)fluoroalkyl, cycloalkyl, or R₁₁ connected to R₆ as alkyl to form a (C₁₋₃)alkyl bridge cyclic structure;
R₁₂═H, F, CH₃, alkyl, substituted alkyl, (C₁₋₃)fluoroalkyl, cycloalkyl, or R₁₂ connected to R₉ as alkyl to form a (C₁₋₃)alkyl bridge cyclic structure;
X═CH₂, O, nothing (for example to provide five membered pyrrolidine ring), CRₐR_{b} (where Rₐ and R_{b}=alkyl, cycloalkyl, Fluoroalkyl); wherein the carbon atom at position 2 of the piperidine or pyrrolidine is preferred in absolute (S)-configuration; in contrast, the carbon atom at position 2 of the of the morpholine ring (when X═O, oxygen) is preferred in absolute (R)-configuration;
Y═O, NH, nothing (R₅′ attached directly to the carbon bearing Z₁ and Z₂ group), CH₂OR₅′, CH₂, NRₐ (wherein Rₐ=alkyl, cycloalkyl, heteroalkyl), or Y could be chosen as a preferred linker as a 5 or 6-membered heteroaryl selected from the group consisting of pyrrolyl, pyrazolyl, triazolyl, oxazolyl, thiazolyl, oxadiazolyl; and,
Z₁, Z₂═H, F, (C₁₋₄)alkyl, (C₁₋₃)fluoroalkyl, (C₁₋₃)fluoroalkoxy, (C₂₋₇)cycloalkyl or independently chosen from one of these groups.

A further embodiment of the disclosure relates to preferred compounds wherein the pyrazole fused six-membered ring is preferred as shown herein according to embodiments of Formulas I-b1 or II-b1, respectively:

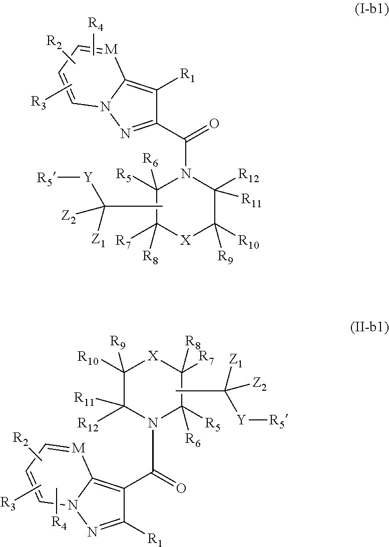

wherein:
R₁ is selected from the group consisting of H, alkyl, alkoxy, cycloalkyl, phenyl, aromatic or aryl, heteroaryl (5-6 membered ring), substituted aromatic or aryl, substituted heteroaryl (5-6 membered ring); or, when R₁ is heteroaryl, R₁ is preferably a 5 or 6-membered heteroaryl selected from the group consisting of pyrazolyl, triazolyl, oxazolyl, thiazolyl, thiophenyl, pyridinyl, pyrimidinyl, pyrazinyl, and pyridazinyl; wherein said aromatic, aryl or heteroaryl is unsubstituted, mono-, or di-substituted, wherein the substituents are independently selected from the group consisting of (C₁₋₄)alkyl, (C₁₋₄)alkoxy, halogen, (C₁₋₃)fluoroalkyl, (C₁₋₃)fluoroalkoxy and (C₃₋₇)cycloalkyl;
R₂, R₃, and R₄ are independently selected from the group consisting of H, halogen (such as F, Cl, Br), alkyl, substituted alkyl, (C₁₋₄)alkyl, (C₁₋₄)alkoxy, (C₁₋₃)fluoroalkyl, (C₁₋₃)fluoroalkoxy and (C₃₋₇)cycloalkyl; wherein each of R₂, R₃ and R₄ is independently and optionally substituted at each substitutable position with up to three (3) substituents independently selected from one, two, or all R₂, R₃ and R₄;
R₅═H, F, CH₃, alkyl, substituted alkyl, (C₁₋₃)fluoroalkyl, cycloalkyl, R₅ replaced with the carbon bearing Y, Z₁ and Z₂, wherein R_{5′} is defined herein;
R_{5′}=aromatic or aryl, heteroaryl (5-6 membered ring), substituted aromatic or aryl, substituted heteroaryl or fused two heteroaryl ring systems (5-6 membered ring); wherein said aromatic, aryl or heteroaryl is unsubstituted, mono-, or di-substituted or tri-substituted, wherein the substituents are independently selected from the group consisting of (C₁₋₄)alkyl, (C₁₋₄)alkoxy, halogen, (C₁₋₃)fluoroalkyl, (C₁₋₃)fluoroalkoxy, (C₃₋₇)cycloalkyl, (C₃₋₇)heterocycloalkyl;
R₆═H, F, CH₃, alkyl, substituted alkyl, (C₁₋₃)fluoroalkyl, cycloalkyl, R₆ connected to either R₁₀ or R₁₁ as alkyl to form a (C₁₋₃)alkyl bridge cyclic structure;
R₇═H, F, CH₃, alkyl, substituted alkyl, (C₁₋₃)fluoroalkyl, cycloalkyl, R₇ replaced with the carbon bearing Y, Z₁ and Z₂, wherein R₅′ is defined herein;
R₈═H, F, CH₃, alkyl, substituted alkyl, (C₁₋₃)fluoroalkyl, cycloalkyl, R₈ connected to either R₁₀. R₁₁ or R₁₂ as alkyl to form a (C₁₋₃)alkyl bridge cyclic structure;
R₉═H, F, CH₃, alkyl, substituted alkyl, (C₁₋₃)fluoroalkyl, cycloalkyl, R₉ connected to either R₆ or R₁₂ as alkyl to form a (C₁₋₃)alkyl bridge cyclic structure;
R₁₀═H, F, CH₃, alkyl, substituted alkyl, (C₁₋₃)fluoroalkyl, cycloalkyl, or R₁₀ connected to R₆ or R₁₁ as alkyl to form a (C₁₋₃)alkyl bridge cyclic structure;
R₁₁═H, F, CH₃, alkyl, substituted alkyl, (C₁₋₃)fluoroalkyl, cycloalkyl, or R₁₁ connected to R₆ as alkyl to form a (C₁₋₃)alkyl bridge cyclic structure;
R₁₂═H, F, CH₃, alkyl, substituted alkyl, (C₁₋₃)fluoroalkyl, cycloalkyl, or R₁₂ connected to R₉ as alkyl to form a (C₁₋₃)alkyl bridge cyclic structure;
X═CH₂, O, nothing (for example to provide five membered pyrrolidine ring), CRₐR_{b} (where Rₐ and R_{b}=alkyl, cycloalkyl, Fluoroalkyl); wherein the carbon atom at position 2 of the piperidine or pyrrolidine is preferred in absolute (S)-configuration; in contrast, the carbon atom at position 2 of the of the morpholine ring (when X═O, oxygen) is preferred in absolute (R)-configuration;
Y═O, NH, nothing (R₅′ attached directly to the carbon bearing Z₁ and Z₂ group), CH₂OR₅′, CH₂, NRₐ (wherein Rₐ=alkyl, cycloalkyl, heteroalkyl), or Y could be chosen as a preferred linker as a 5 or 6-membered heteroaryl selected from the group consisting of pyrrolyl, pyrazolyl, triazolyl, oxazolyl, thiazolyl, oxadiazolyl; and,
Z₁, Z₂═H, F, (C₁₋₄)alkyl, (C₁₋₃)fluoroalkyl, (C₁₋₃)fluoroalkoxy, (C₂₋₇)cycloalkyl or independently;
wherein the preferred groups are:
M=Carbon, CH, CHR₂, CHR₃, CR₂R₃, CR₂, CR₃, CR₄, and N.

A further embodiment of the disclosure relates to preferred compounds wherein the pyrazole fused six-membered ring is preferred as shown herein according to embodiments of Formulas I-b2 or II-b2, respectively:

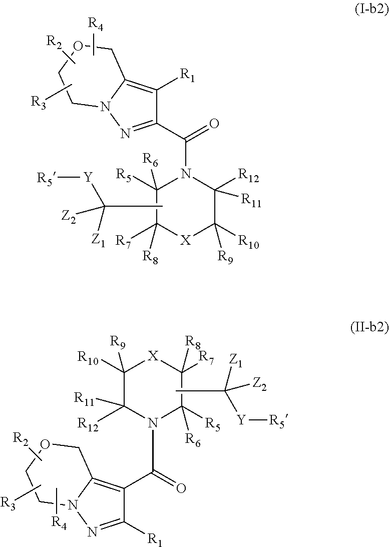

wherein:
R₁ is selected from the group consisting of H, alkyl, alkoxy, cycloalkyl, phenyl, aromatic or aryl, heteroaryl (5-6 membered ring), substituted aromatic or aryl, substituted heteroaryl (5-6 membered ring); or, when R₁ is heteroaryl, R₁ is preferably a 5 or 6-membered heteroaryl selected from the group consisting of pyrazolyl, triazolyl, oxazolyl, thiazolyl, thiophenyl, pyridinyl, pyrimidinyl, pyrazinyl, and pyridazinyl; wherein said aromatic, aryl or heteroaryl is unsubstituted, mono-, or di-substituted, wherein the substituents are independently selected from the group consisting of (C₁₋₄)alkyl, (C₁₋₄)alkoxy, halogen, (C₁₋₃)fluoroalkyl, (C₁₋₃)fluoroalkoxy, and (C₃₋₇)cycloalkyl;
R₂, R₃, and R₄ are independently selected from the group consisting of H, halogen (such as F, Cl, Br), alkyl, substituted alkyl, (C₁₋₄)alkyl, (C₁₋₄)alkoxy, (C₁₋₃)fluoroalkyl, (C₁₋₃)fluoroalkoxy and (C₃₋₇)cycloalkyl; wherein each of R₂, R₃ and R₄ is independently and optionally substituted at each substitutable position with up to three (3) substituents independently selected from one, two, or all R₂, R₃ and R₄;
R₅═H, F, CH₃, alkyl, substituted alkyl, (C₁₋₃)fluoroalkyl, cycloalkyl, R₅ replaced with the carbon bearing Y, Z₁ and Z₂, wherein R₅ is defined herein;
R_{5′}=aromatic or aryl, heteroaryl (5-6 membered ring), substituted aromatic or aryl, substituted heteroaryl or fused two heteroaryl ring systems (5-6 membered ring); wherein said aromatic, aryl or heteroaryl is unsubstituted, mono-, or di-substituted or tri-substituted, wherein the substituents are independently selected from the group consisting of (C₁₋₄)alkyl, (C₁₋₄)alkoxy, halogen, (C₁₋₃)fluoroalkyl, (C₁₋₃)fluoroalkoxy, (C₃₋₇)cycloalkyl, (C₃₋₇)heterocycloalkyl;
R₆═H, F, CH₃, alkyl, substituted alkyl, (C₁₋₃)fluoroalkyl, cycloalkyl, R₆ connected to either R₁₀ or R₁₁ as alkyl to form a (C₁₋₃)alkyl bridge cyclic structure;
R₇═H, F, CH₃, alkyl, substituted alkyl, (C₁₋₃)fluoroalkyl, cycloalkyl, R₇ replaced with the carbon bearing Y, Z₁ and Z₂, wherein R_{5′} is defined herein;
R₈═H, F, CH₃, alkyl, substituted alkyl, (C₁₋₃)fluoroalkyl, cycloalkyl, R₈ connected to either R₁₀. R₁₁ or R₁₂ as alkyl to form a (C₁₋₃)alkyl bridge cyclic structure;
R₉═H, F, CH₃, alkyl, substituted alkyl, (C₁₋₃)fluoroalkyl, cycloalkyl, R₉ connected to either R₆ or R₁₂ as alkyl to form a (C₁₋₃)alkyl bridge cyclic structure;
R₁₀═H, F, CH₃, alkyl, substituted alkyl, (C₁₋₃)fluoroalkyl, cycloalkyl, or R₁₀ connected to R₆ or R₁₁ as alkyl to form a (C₁₋₃)alkyl bridge cyclic structure;
R₁₁═H, F, CH₃, alkyl, substituted alkyl, (C₁₋₃)fluoroalkyl, cycloalkyl, or R₁₁ connected to R₆ as alkyl to form a (C₁₋₃)alkyl bridge cyclic structure;
R₁₂═H, F, CH₃, alkyl, substituted alkyl, (C₁₋₃)fluoroalkyl, cycloalkyl, or R₁₂ connected to R₉ as alkyl to form a (C₁₋₃)alkyl bridge cyclic structure;
X═CH₂, O, nothing (for example to provide five membered pyrrolidine ring), CRₐR_{b} (where Rₐ and R_{b}=alkyl, cycloalkyl, Fluoroalkyl); wherein the carbon atom at position 2 of the piperidine or pyrrolidine is preferred in absolute (S)-configuration; in contrast, the carbon atom at position 2 of the of the morpholine ring (when X═O, oxygen) is preferred in absolute (R)-configuration;
Y═O, NH, nothing (R₅′ attached directly to the carbon bearing Z₁ and Z₂ group), CH₂OR₅′, CH₂, NRₐ (wherein Rₐ=alkyl, cycloalkyl, heteroalkyl), or Y could be chosen as a preferred linker as a 5 or 6-membered heteroaryl selected from the group consisting of pyrrolyl, pyrazolyl, triazolyl, oxazolyl, thiazolyl, oxadiazolyl; and,
Z₁, Z₂═H, F, (C₁₋₄)alkyl, (C₁₋₃)fluoroalkyl, (C₁₋₃)fluoroalkoxy, (C₂₋₇)cycloalkyl or independently chosen from one of these groups.

A further embodiment of the disclosure relates to preferred compounds wherein the pyrazole fused six-membered ring is preferred as shown herein according to the embodiments of Formulas I-b3 or II-b3, respectively:

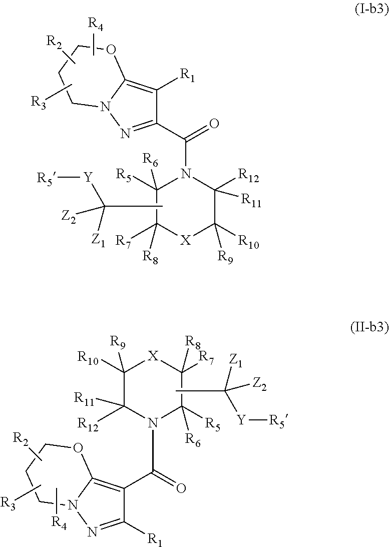

wherein:
R₁ is selected from the group consisting of H, alkyl, alkoxy, cycloalkyl, phenyl, aromatic or aryl, heteroaryl (5-6 membered ring), substituted aromatic or aryl, substituted heteroaryl (5-6 membered ring); or, when R₁ is heteroaryl, R₁ is preferably a 5 or 6-membered heteroaryl selected from the group consisting of pyrazolyl, triazolyl, oxazolyl, thiazolyl, thiophenyl, pyridinyl, pyrimidinyl, pyrazinyl, and pyridazinyl; wherein said aromatic, aryl or heteroaryl is unsubstituted, mono-, or di-substituted, wherein the substituents are independently selected from the group consisting of (C₁₋₄)alkyl, (C₁₋₄)alkoxy, halogen, (C₁₋₃)fluoroalkyl, (C₁₋₃)fluoroalkoxy and (C₃₋₇)cycloalkyl;
R₂, R₃, and R₄ are independently selected from the group consisting of H, halogen (such as F, Cl, Br), alkyl, substituted alkyl, (C₁₋₄)alkyl, (C₁₋₄)alkoxy, (C₁₋₃)fluoroalkyl, (C₁₋₃)fluoroalkoxy and (C₃₋₇)cycloalkyl; wherein each of R₂, R₃ and R₄ is independently and optionally substituted at each substitutable position with up to three (3) substituents independently selected from one, two, or all R₂, R₃ and R₄;
R₅═H, F, CH₃, alkyl, substituted alkyl, (C₁₋₃)fluoroalkyl, cycloalkyl, R₅ replaced with the carbon bearing Y, Z₁ and Z₂, wherein R₅ is defined herein;
R_{5′}=aromatic or aryl, heteroaryl (5-6 membered ring), substituted aromatic or aryl, substituted heteroaryl or fused two heteroaryl ring systems (5-6 membered ring); wherein said aromatic, aryl or heteroaryl is unsubstituted, mono-, or di-substituted or tri-substituted, wherein the substituents are independently selected from the group consisting of (C₁₋₄)alkyl, (C₁₋₄)alkoxy, halogen, (C₁₋₃)fluoroalkyl, (C₁₋₃)fluoroalkoxy, (C₃₋₇)cycloalkyl, (C₃₋₇)heterocycloalkyl;
R₆═H, F, CH₃, alkyl, substituted alkyl, (C₁₋₃)fluoroalkyl, cycloalkyl, R₆ connected to either R₁₀ or R₁₁ as alkyl to form a (C₁₋₃)alkyl bridge cyclic structure;
R₇═H, F, CH₃, alkyl, substituted alkyl, (C₁₋₃)fluoroalkyl, cycloalkyl, R₇ replaced with the carbon bearing Y, Z₁ and Z₂, wherein R_{5′} is defined herein;
R₈═H, F, CH₃, alkyl, substituted alkyl, (C₁₋₃)fluoroalkyl, cycloalkyl, R₈ connected to either R₁₀, R₁₁ or R₁₂ as alkyl to form a (C₁₋₃)alkyl bridge cyclic structure;
R₉═H, F, CH₃, alkyl, substituted alkyl, (C₁₋₃)fluoroalkyl, cycloalkyl, R₉ connected to either R₆ or R₁₂ as alkyl to form a (C₁₋₃)alkyl bridge cyclic structure;
R₁₀═H, F, CH₃, alkyl, substituted alkyl, (C₁₋₃)fluoroalkyl, cycloalkyl, or R₁₀ connected to R₆ or R₁₁ as alkyl to form a (C₁₋₃)alkyl bridge cyclic structure;
R₁₁═H, F, CH₃, alkyl, substituted alkyl, (C₁₋₃)fluoroalkyl, cycloalkyl, or R₁₁ connected to R₆ as alkyl to form a (C₁₋₃)alkyl bridge cyclic structure;
R₁₂═H, F, CH₃, alkyl, substituted alkyl, (C₁₋₃)fluoroalkyl, cycloalkyl, or R₁₂ connected to R₉ as alkyl to form a (C₁₋₃)alkyl bridge cyclic structure;
X═CH₂, O, nothing (for example to provide five membered pyrrolidine ring), CRₐR_{b} (where Rₐ and R_{b}=alkyl, cycloalkyl, Fluoroalkyl); wherein the carbon atom at position 2 of the piperidine or pyrrolidine is preferred in absolute (S)-configuration; in contrast, the carbon atom at position 2 of the of the morpholine ring (when X═O, oxygen) is preferred in absolute (R)-configuration;
Y═O, NH, nothing (R₅′ attached directly to the carbon bearing Z₁ and Z₂ group), CH₂OR₅′, CH₂, NRₐ (wherein Rₐ=alkyl, cycloalkyl, heteroalkyl), or Y could be chosen as a preferred linker as a 5 or 6-membered heteroaryl selected from the group consisting of pyrrolyl, pyrazolyl, triazolyl, oxazolyl, thiazolyl, oxadiazolyl; and,
Z₁, Z₂═H, F, (C₁₋₄)alkyl, (C₁₋₃)fluoroalkyl, (C₁₋₃)fluoroalkoxy, (C₂₋₇)cycloalkyl or independently chosen from one of these groups.

In a more specific (i.e., preferred) embodiment of the disclosure as it relates to compounds wherein the stereogenic centers and main scaffold rings are preferred as shown in the exemplification Formula as shown herein according to embodiments of Formulas I-a4, I-a5, I-a6, II-a4, II-a5, or II-a6, respectively:

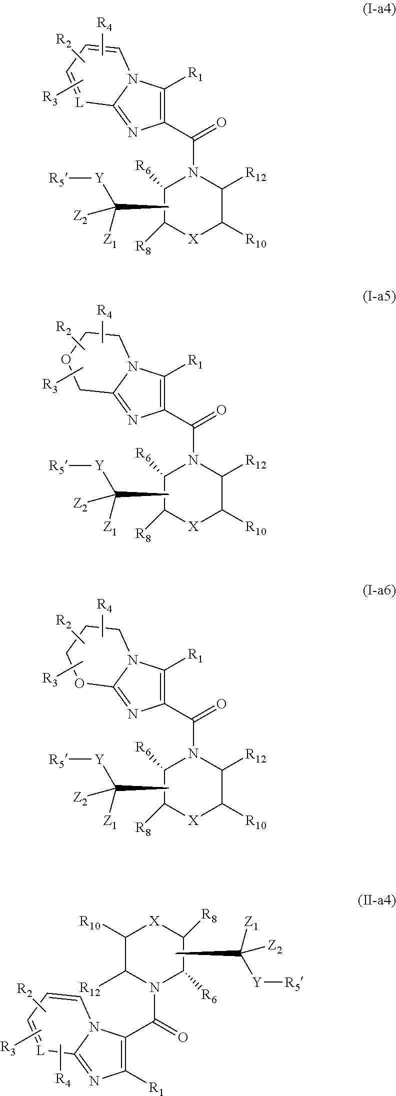

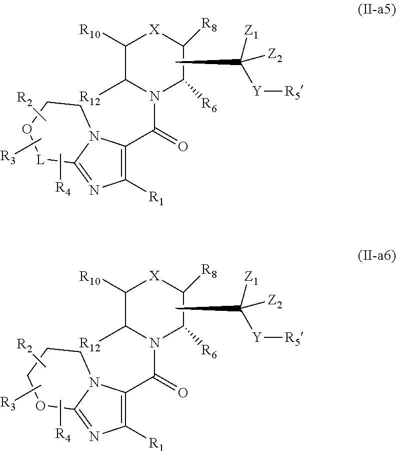

wherein:
R₁ is selected from the group consisting of H, alkyl, alkoxy, cycloalkyl, phenyl, aromatic or aryl, heteroaryl (5-6 membered ring), substituted aromatic or aryl, substituted heteroaryl (5-6 membered ring); or, when R₁ is heteroaryl, R₁ is preferably a 5 or 6-membered heteroaryl selected from the group consisting of pyrazolyl, triazolyl, oxazolyl, thiazolyl, thiophenyl, pyridinyl, pyrimidinyl, pyrazinyl, and pyridazinyl; wherein said aromatic, aryl or heteroaryl is unsubstituted, mono-, or di-substituted, wherein the substituents are independently selected from the group consisting of (C₁₋₄)alkyl, (C₁₋₄)alkoxy, halogen, (C₁₋₃)fluoroalkyl, (C₁₋₃)fluoroalkoxy and (C₃₋₇)cycloalkyl;
R₂, R₃, and R₄ are independently selected from the group consisting of H, halogen (such as F, Cl, Br), alkyl, substituted alkyl, (C₁₋₄)alkyl, (C₁₋₄)alkoxy, (C₁₋₃)fluoroalkyl, (C₁₋₃)fluoroalkoxy and (C₃₋₇)cycloalkyl; wherein each of R₂, R₃ and R₄ is independently and optionally substituted at each substitutable position with up to three (3) substituents independently selected from one, two, or all R₂, R₃ and R₄;
R₅ is H or replaced with the carbon bearing Y, Z₁ and Z₂, wherein R_{5′} is defined herein;
R_{5′}=aromatic or aryl, heteroaryl (5-6 membered ring), substituted aromatic or aryl, substituted heteroaryl or fused two heteroaryl ring systems (5-6 membered ring); wherein said aromatic, aryl or heteroaryl is unsubstituted, mono-, or di-substituted or tri-substituted, wherein the substituents are independently selected from the group consisting of (C₁₋₄)alkyl, (C₁₋₄)alkoxy, halogen, (C₁₋₃)fluoroalkyl, (C₁₋₃)fluoroalkoxy, (C₃₋₇)cycloalkyl, (C₃₋₇)heterocycloalkyl;
R₆═H, F, CH₃, alkyl, substituted alkyl, (C₁₋₃)fluoroalkyl, cycloalkyl, R₆ connected to either R₁₀ or R₁₁ as alkyl to form a (C₁₋₃)alkyl bridge cyclic structure;
R₇ is H or replaced with the carbon bearing Y, Z₁ and Z₂, wherein R_{5′} is defined herein;
R₈═H, F, CH₃, alkyl, substituted alkyl, (C₁₋₃)fluoroalkyl, cycloalkyl, R₈ connected to either R₁₀, R₁₁ or R₁₂ as alkyl to form a (C₁₋₃)alkyl bridge cyclic structure;
R₉═H, F, CH₃, alkyl, substituted alkyl, (C₁₋₃)fluoroalkyl, cycloalkyl, R₉ connected to either R₆ or R₁₂ as alkyl to form a (C₁₋₃)alkyl bridge cyclic structure;
R₁₀═H, F, CH₃, alkyl, substituted alkyl, (C₁₋₃)fluoroalkyl, cycloalkyl, or R₁₀ connected to R₆ or R₁₁ as alkyl to form a (C₁₋₃)alkyl bridge cyclic structure;
R₁₁═H, F, CH₃, alkyl, substituted alkyl, (C₁₋₃)fluoroalkyl, cycloalkyl, or R₁₁ connected to R₆ as alkyl to form a (C₁₋₃)alkyl bridge cyclic structure;
R₁₂═H, F, CH₃, alkyl, substituted alkyl, (C₁₋₃)fluoroalkyl, cycloalkyl, or R₁₂ connected to R₉ as alkyl to form a (C₁₋₃)alkyl bridge cyclic structure;
X═CH₂, O, nothing (for example to provide five membered pyrrolidine ring), CRₐR_{b} (where Rₐ and R_{b}=alkyl, cycloalkyl, Fluoroalkyl); wherein the carbon atom at position 2 of the piperidine or pyrrolidine is preferred in absolute (S)-configuration; in contrast, the carbon atom at position 2 of the of the morpholine ring (when X═O, oxygen) is preferred in absolute (R)-configuration;
Y═O, NH, nothing (R₅′ attached directly to the carbon bearing Z₁ and Z₂ group), CH₂OR₅′, CH₂, NRₐ (wherein Rₐ=alkyl, cycloalkyl, heteroalkyl), or Y could be chosen as a preferred linker as a 5 or 6-membered heteroaryl selected from the group consisting of pyrrolyl, pyrazolyl, triazolyl, oxazolyl, thiazolyl, oxadiazolyl;
Z₁, Z₂═H, F, (C₁₋₄)alkyl, (C₁₋₃)fluoroalkyl, (C₁₋₃)fluoroalkoxy, (C₂₋₇)cycloalkyl or independently chosen from one of these groups; and,
L=Carbon, CH, CHR₂, CHR₃, CR₂R₃, CR₂, CR₃, CR₄, and N.

In a more specific (i.e., preferred) embodiment of the disclosure as it relates to compounds wherein the stereogenic centers and main scaffold rings are preferred as shown in the exemplification Formula as shown herein according to the embodiments of Formulas I-b4, I-b5, I-b6, II-b4, II-b5, or II-b6, respectively:

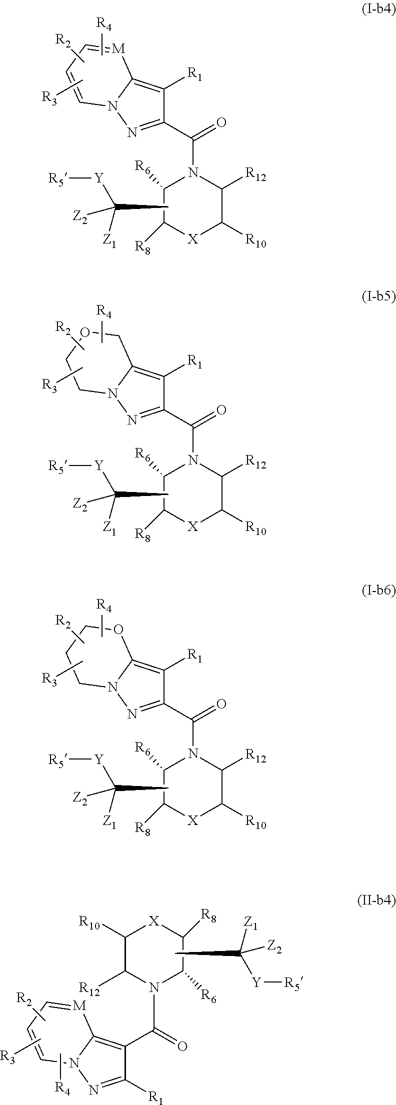

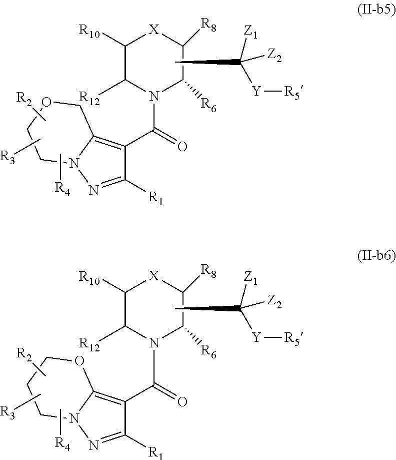

wherein:
R₁ is selected from the group consisting of H, alkyl, alkoxy, cycloalkyl, phenyl, aromatic or aryl, heteroaryl (5-6 membered ring), substituted aromatic or aryl, substituted heteroaryl (5-6 membered ring); or, when R₁ is heteroaryl, R₁ is preferably a 5 or 6-membered heteroaryl selected from the group consisting of pyrazolyl, triazolyl, oxazolyl, thiazolyl, thiophenyl, pyridinyl, pyrimidinyl, pyrazinyl, and pyridazinyl; wherein said aromatic, aryl or heteroaryl is unsubstituted, mono-, or di-substituted, wherein the substituents are independently selected from the group consisting of (C₁₋₄)alkyl, (C₁₋₄)alkoxy, halogen, (C₁₋₃)fluoroalkyl, (C₁₋₃)fluoroalkoxy and (C₃₋₇)cycloalkyl;
R₂, R₃, and R₄ are independently selected from the group consisting of H, halogen (such as F, Cl, Br), alkyl, substituted alkyl, (C₁₋₄)alkyl, (C₁₋₄)alkoxy, (C₁₋₃)fluoroalkyl, (C₁₋₃)fluoroalkoxy and (C₃₋₇)cycloalkyl; wherein each of R₂, R₃ and R₄ is independently and optionally substituted at each substitutable position with up to three (3) substituents independently selected from one, two, or all R₂, R₃ and R₄;
R₅ is H or replaced with the carbon bearing Y, Z₁ and Z₂, wherein R_{5′} is defined herein;
R_{5′}=aromatic or aryl, heteroaryl (5-6 membered ring), substituted aromatic or aryl, substituted heteroaryl or fused two heteroaryl ring systems (5-6 membered ring); wherein said aromatic, aryl or heteroaryl is unsubstituted, mono-, or di-substituted or tri-substituted, wherein the substituents are independently selected from the group consisting of (C₁₋₄)alkyl, (C₁₋₄)alkoxy, halogen, (C₁₋₃)fluoroalkyl, (C₁₋₃)fluoroalkoxy, (C₃₋₇)cycloalkyl, (C₃₋₇)heterocycloalkyl;
R₆═H, F, CH₃, alkyl, substituted alkyl, (C₁₋₃)fluoroalkyl, cycloalkyl, R₆ connected to either R₁₀ or R₁₁ as alkyl to form a (C₁₋₃)alkyl bridge cyclic structure;
R₇H or is replaced with the carbon bearing Y, Z1 and Z₂, wherein R_{5′} is defined herein;
R₈═H, F, CH₃, alkyl, substituted alkyl, (C₁₋₃)fluoroalkyl, cycloalkyl, R₈ connected to either R₁₀, R₁₁ or R₁₂ as alkyl to form a (C₁₋₃)alkyl bridge cyclic structure;
R₉═H, F, CH₃, alkyl, substituted alkyl, (C₁₋₃)fluoroalkyl, cycloalkyl, R₉ connected to either R₆ or R₁₂ as alkyl to form a (C₁₋₃)alkyl bridge cyclic structure;
R₁₀═H, F, CH₃, alkyl, substituted alkyl, (C₁₋₃)fluoroalkyl, cycloalkyl, or R₁₀ connected to R₆ or R₁₁ as alkyl to form a (C₁₋₃)alkyl bridge cyclic structure;
R₁₁═H, F, CH₃, alkyl, substituted alkyl, (C₁₋₃)fluoroalkyl, cycloalkyl, or R₁₁ connected to R₆ as alkyl to form a (C₁₋₃)alkyl bridge cyclic structure;
R₁₂═H, F, CH₃, alkyl, substituted alkyl, (C₁₋₃)fluoroalkyl, cycloalkyl, or R₁₂ connected to R₉ as alkyl to form a (C₁₋₃)alkyl bridge cyclic structure;
X═CH₂, O, nothing (for example to provide five membered pyrrolidine ring), CRₐR_{b} (where Rₐ and R_{b}=alkyl, cycloalkyl, Fluoroalkyl); wherein the carbon atom at position 2 of the piperidine or pyrrolidine is preferred in absolute (S)-configuration; in contrast, the carbon atom at position 2 of the of the morpholine ring (when X═O, oxygen) is preferred in absolute (R)-configuration;
Y═O, NH, nothing (R₅′ attached directly to the carbon bearing Z1 and Z₂ group), CH₂OR_{5′}, CH₂, NRₐ (wherein Rₐ=alkyl, cycloalkyl, heteroalkyl), or Y could be chosen as a preferred linker as a 5 or 6-membered heteroaryl selected from the group consisting of pyrrolyl, pyrazolyl, triazolyl, oxazolyl, thiazolyl, oxadiazolyl; and,
Z₁, Z₂═H, F, (C₁₋₄)alkyl, (C₁₋₃)fluoroalkyl, (C₁₋₃)fluoroalkoxy, (C₂₋₇)cycloalkyl or independently chosen from one of these groups;
wherein the preferred groups are:
M=Carbon, OH, CHR₂, CHR₃, CR₂R₃, CR₂, CR₃, CR₄, and N.

Exemplary preferred compounds of Formulas I, II, I-a, and/or II-b and also being comprised within the subFormulas which are included in the main Formulas, and in preferred embodiments as shown in Table 1 below.

Preferred compounds of this disclosure are those presented below as Examples 1-263. These compounds are shown in the Examples section of this disclosure and shown below. To the extent any of the Example number and/or structure of the compounds shown in this Table 1 contradict those presented in the working Examples section (Table 3), the numbering and/or structure presented in Table 3 controls.

TABLE 1

| Ex. | Compound Structure |
|---|---|
| 1 | 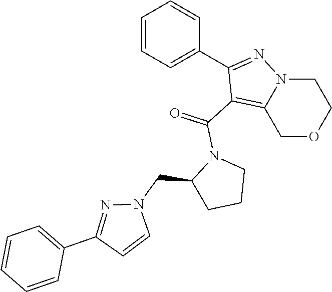 [SMILES: O=C(c1c(-c2ccccc2)nn2c1COCC2)N1CCC[C@H]1Cn1ccc(-c2ccccc2)n1] |
| 2 | 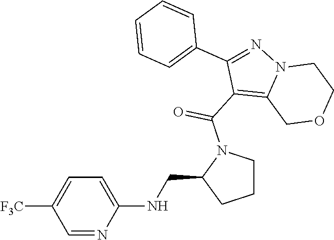 [SMILES: O=C(c1c(-c2ccccc2)nn2c1COCC2)N1CCC[C@H]1CNc1ccc(C(F)(F)F)cn1] |
| 3 | 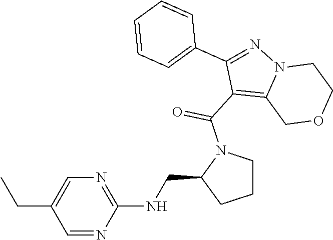 [SMILES: CCc1cnc(NC[C@@H]2CCCN2C(=O)c2c(-c3ccccc3)nn3c2COCC3)nc1] |
| 4 | 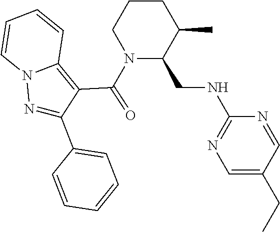 [SMILES: CCc1cnc(NC[C@@H]2[C@H](C)CCCN2C(=O)c2c(-c3ccccc3)nn3ccccc23)nc1] |
| 5 | 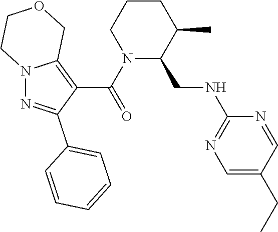 [SMILES: CCc1cnc(NC[C@@H]2[C@H](C)CCCN2C(=O)c2c(-c3ccccc3)nn3c2COCC3)nc1] |
| 6 | 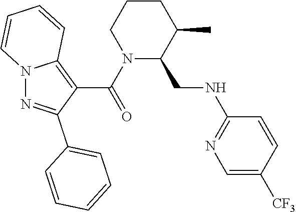 [SMILES: C[C@@H]1CCCN(C(=O)c2c(-c3ccccc3)nn3ccccc23)[C@@H]1CNc1ccc(C(F)(F)F)cn1] |
| 7 | 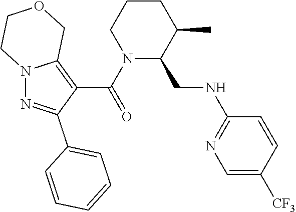 [SMILES: C[C@@H]1CCCN(C(=O)c2c(-c3ccccc3)nn3c2COCC3)[C@@H]1CNc1ccc(C(F)(F)F)cn1] |
| 8 | 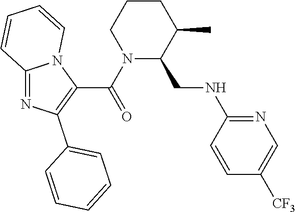 [SMILES: C[C@@H]1CCCN(C(=O)c2c(-c3ccccc3)nc3ccccn23)[C@@H]1CNc1ccc(C(F)(F)F)cn1] |
| 9 | 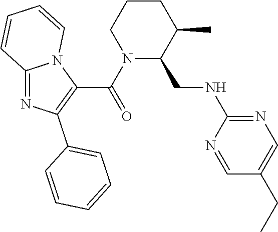 [SMILES: CCc1cnc(NC[C@@H]2[C@H](C)CCCN2C(=O)c2c(-c3ccccc3)nc3ccccn23)nc1] |
| 10 | 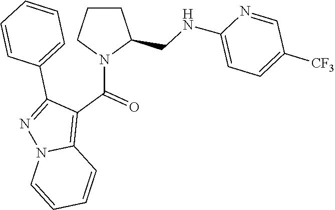 [SMILES: O=C(c1c(-c2ccccc2)nn2ccccc12)N1CCC[C@H]1CNc1ccc(C(F)(F)F)cn1] |
| 11 | 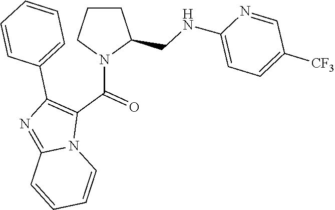 [SMILES: O=C(c1c(-c2ccccc2)nc2ccccn12)N1CCC[C@H]1CNc1ccc(C(F)(F)F)cn1] |
| 12 | 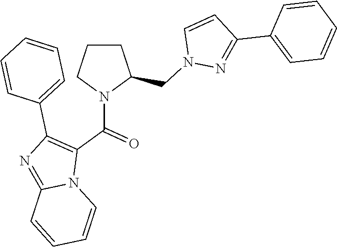 [SMILES: O=C(c1c(-c2ccccc2)nc2ccccn12)N1CCC[C@H]1Cn1ccc(-c2ccccc2)n1] |
| 13 | 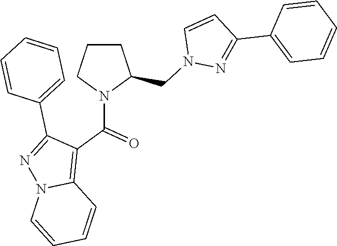 [SMILES: O=C(c1c(-c2ccccc2)nn2ccccc12)N1CCC[C@H]1Cn1ccc(-c2ccccc2)n1] |
| 14 | 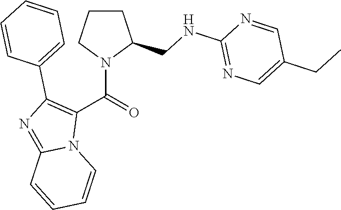 [SMILES: CCc1cnc(NC[C@@H]2CCCN2C(=O)c2c(-c3ccccc3)nc3ccccn23)nc1] |
| 15 | 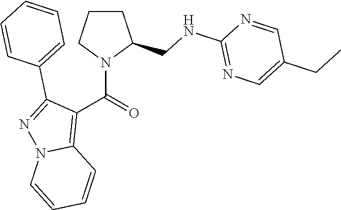 [SMILES: CCc1cnc(NC[C@@H]2CCCN2C(=O)c2c(-c3ccccc3)nn3ccccc23)nc1] |
| 16 | 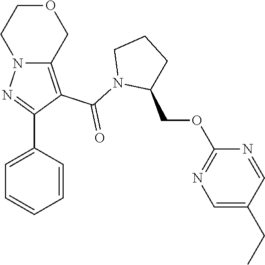 [SMILES: CCc1cnc(OC[C@@H]2CCCN2C(=O)c2c(-c3ccccc3)nn3c2COCC3)nc1] |
| 17 | 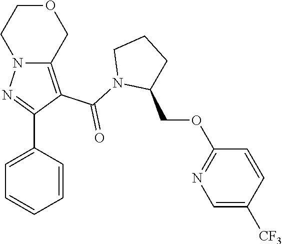 [SMILES: O=C(c1c(-c2ccccc2)nn2c1COCC2)N1CCC[C@H]1COc1ccc(C(F)(F)F)cn1] |
| 18 | 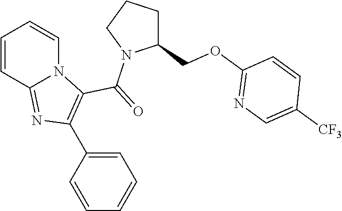 [SMILES: O=C(c1c(-c2ccccc2)nc2ccccn12)N1CCC[C@H]1COc1ccc(C(F)(F)F)cn1] |
| 19 | 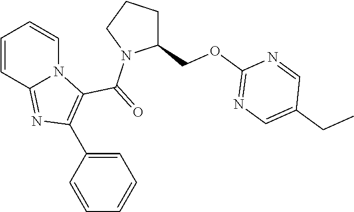 [SMILES: CCc1cnc(OC[C@@H]2CCCN2C(=O)c2c(-c3ccccc3)nc3ccccn23)nc1] |
| 20 | 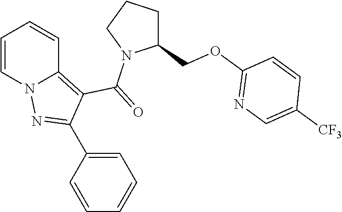 [SMILES: O=C(c1c(-c2ccccc2)nn2ccccc12)N1CCC[C@H]1COc1ccc(C(F)(F)F)cn1] |
| 21 | 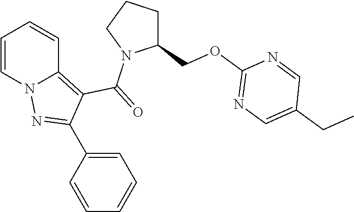 [SMILES: CCc1cnc(OC[C@@H]2CCCN2C(=O)c2c(-c3ccccc3)nn3ccccc23)nc1] |
| 22 | 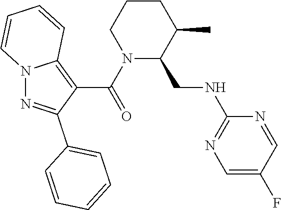 [SMILES: C[C@@H]1CCCN(C(=O)c2c(-c3ccccc3)nn3ccccc23)[C@@H]1CNc1ncc(F)cn1] |
| 23 | 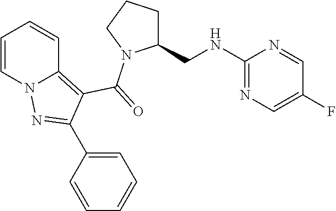 [SMILES: O=C(c1c(-c2ccccc2)nn2ccccc12)N1CCC[C@H]1CNc1ncc(F)cn1] |
| 24 | 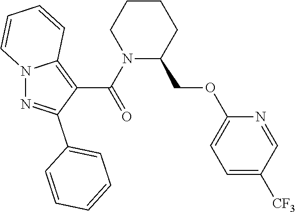 [SMILES: O=C(c1c(-c2ccccc2)nn2ccccc12)N1CCCC[C@H]1COc1ccc(C(F)(F)F)cn1] |
| 25 | 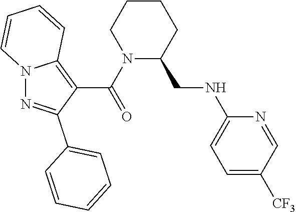 [SMILES: O=C(c1c(-c2ccccc2)nn2ccccc12)N1CCCC[C@H]1CNc1ccc(C(F)(F)F)cn1] |
| 26 | 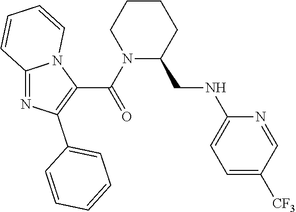 [SMILES: O=C(c1c(-c2ccccc2)nc2ccccn12)N1CCCC[C@H]1CNc1ccc(C(F)(F)F)cn1] |
| 27 | 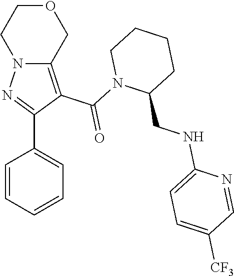 [SMILES: O=C(c1c(-c2ccccc2)nn2c1COCC2)N1CCCC[C@H]1CNc1ccc(C(F)(F)F)cn1] |
| 28 | 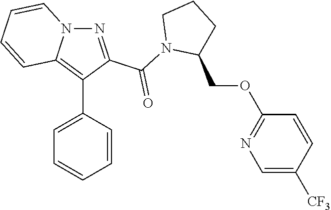 [SMILES: O=C(c1nn2ccccc2c1-c1ccccc1)N1CCC[C@H]1COc1ccc(C(F)(F)F)cn1] |
| 29 | 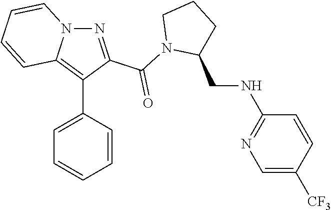 [SMILES: O=C(c1nn2ccccc2c1-c1ccccc1)N1CCC[C@H]1CNc1ccc(C(F)(F)F)cn1] |
| 30 | 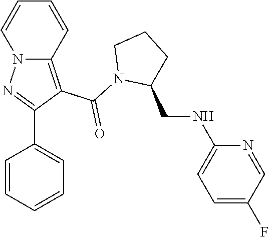 [SMILES: O=C(c1c(-c2ccccc2)nn2ccccc12)N1CCC[C@H]1CNc1ccc(F)cn1] |
| 31 | 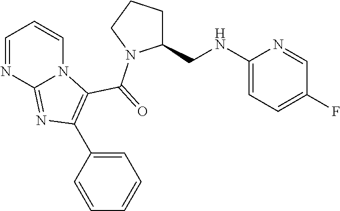 [SMILES: O=C(c1c(-c2ccccc2)nc2ncccn12)N1CCC[C@H]1CNc1ccc(F)cn1] |
| 32 | 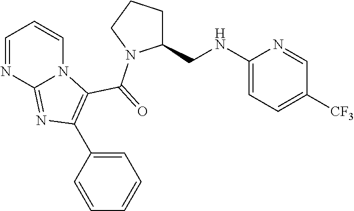 [SMILES: O=C(c1c(-c2ccccc2)nc2ncccn12)N1CCC[C@H]1CNc1ccc(C(F)(F)F)cn1] |
| 33 | 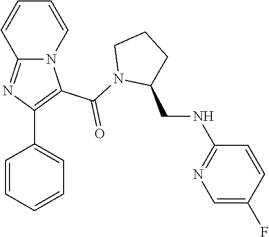 [SMILES: O=C(c1c(-c2ccccc2)nc2ccccn12)N1CCC[C@H]1CNc1ccc(F)cn1] |
| 34 | 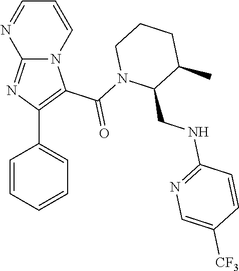 [SMILES: C[C@@H]1CCCN(C(=O)c2c(-c3ccccc3)nc3ncccn23)[C@@H]1CNc1ccc(C(F)(F)F)cn1] |
| 35 | 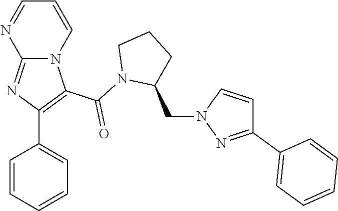 [SMILES: O=C(c1c(-c2ccccc2)nc2ncccn12)N1CCC[C@H]1Cn1ccc(-c2ccccc2)n1] |
| 36 | 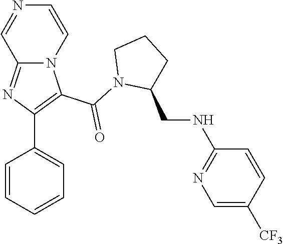 [SMILES: O=C(c1c(-c2ccccc2)nc2cnccn12)N1CCC[C@H]1CNc1ccc(C(F)(F)F)cn1] |
| 37 | 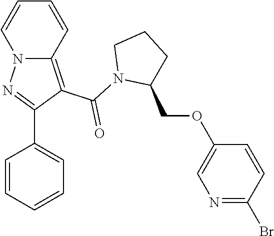 [SMILES: O=C(c1c(-c2ccccc2)nn2ccccc12)N1CCC[C@H]1COc1ccc(Br)nc1] |
| 38 | 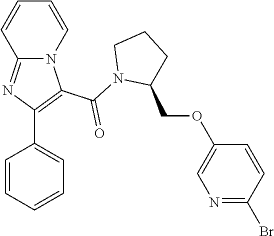 [SMILES: O=C(c1c(-c2ccccc2)nc2ccccn12)N1CCC[C@H]1COc1ccc(Br)nc1] |
| 39 | 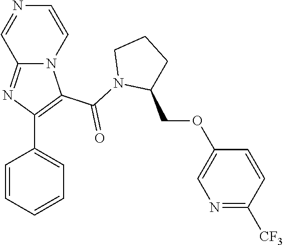 [SMILES: O=C(c1c(-c2ccccc2)nc2cnccn12)N1CCC[C@H]1COc1ccc(C(F)(F)F)nc1] |
| 40 | 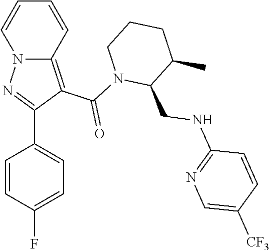 [SMILES: C[C@@H]1CCCN(C(=O)c2c(-c3ccc(F)cc3)nn3ccccc23)[C@@H]1CNc1ccc(C(F)(F)F)cn1] |
| 41 | 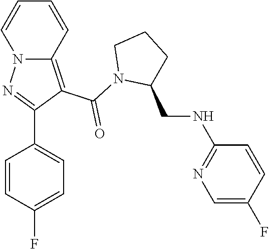 [SMILES: O=C(c1c(-c2ccc(F)cc2)nn2ccccc12)N1CCC[C@H]1CNc1ccc(F)cn1] |
| 42 | 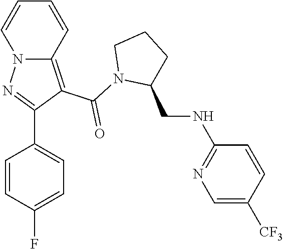 [SMILES: O=C(c1c(-c2ccc(F)cc2)nn2ccccc12)N1CCC[C@H]1CNc1ccc(C(F)(F)F)cn1] |
| 43 | 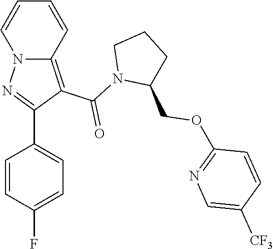 [SMILES: O=C(c1c(-c2ccc(F)cc2)nn2ccccc12)N1CCC[C@H]1COc1ccc(C(F)(F)F)cn1] |
| 44 | 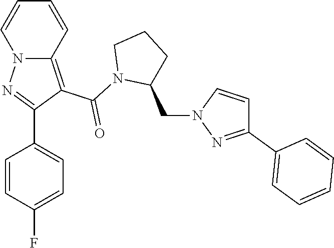 [SMILES: O=C(c1c(-c2ccc(F)cc2)nn2ccccc12)N1CCC[C@H]1Cn1ccc(-c2ccccc2)n1] |
| 45 | 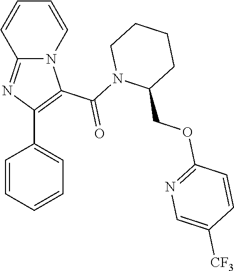 [SMILES: O=C(c1c(-c2ccccc2)nc2ccccn12)N1CCCC[C@H]1COc1ccc(C(F)(F)F)cn1] |
| 46 | 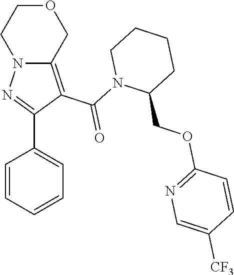 [SMILES: O=C(c1c(-c2ccccc2)nn2c1COCC2)N1CCCC[C@H]1COc1ccc(C(F)(F)F)cn1] |
| 47 | 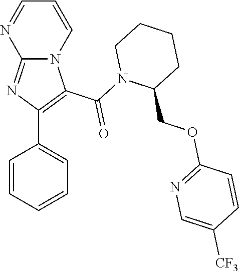 [SMILES: O=C(c1c(-c2ccccc2)nc2ncccn12)N1CCCC[C@H]1COc1ccc(C(F)(F)F)cn1] |
| 48 | 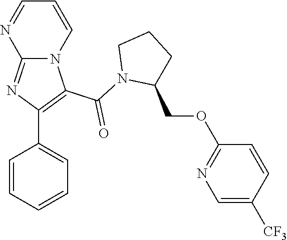 [SMILES: O=C(c1c(-c2ccccc2)nc2ncccn12)N1CCC[C@H]1COc1ccc(C(F)(F)F)cn1] |
| 49 | 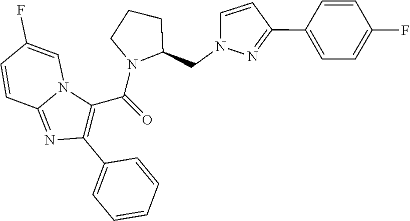 [SMILES: O=C(c1c(-c2ccccc2)nc2ccc(F)cn12)N1CCC[C@H]1Cn1ccc(-c2ccc(F)cc2)n1] |
| 50 | 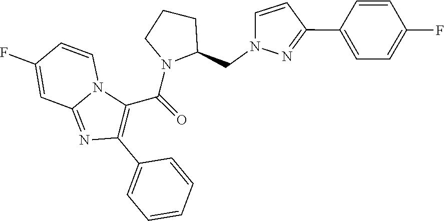 [SMILES: O=C(c1c(-c2ccccc2)nc2cc(F)ccn12)N1CCC[C@H]1Cn1ccc(-c2ccc(F)cc2)n1] |
| 51 | 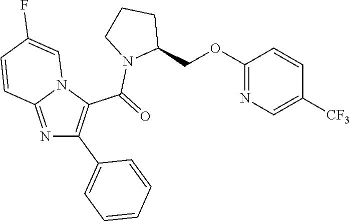 [SMILES: O=C(c1c(-c2ccccc2)nc2ccc(F)cn12)N1CCC[C@H]1COc1ccc(C(F)(F)F)cn1] |
| 52 | 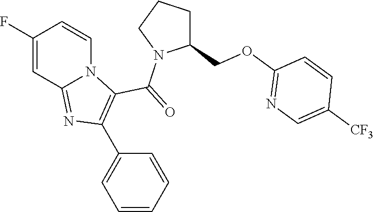 [SMILES: O=C(c1c(-c2ccccc2)nc2cc(F)ccn12)N1CCC[C@H]1COc1ccc(C(F)(F)F)cn1] |
| 53 | 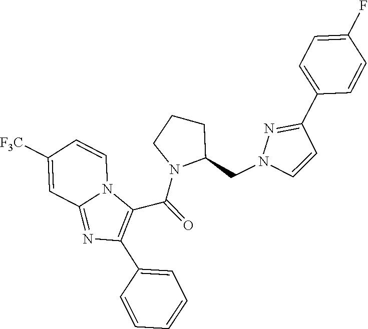 [SMILES: O=C(c1c(-c2ccccc2)nc2cc(C(F)(F)F)ccn12)N1CCC[C@H]1Cn1ccc(-c2ccc(F)cc2)n1] |
| 54 | 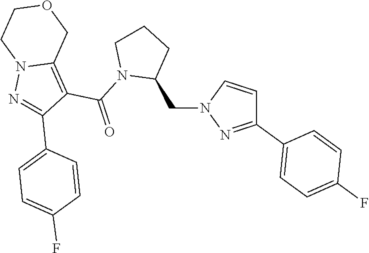 [SMILES: O=C(c1c(-c2ccc(F)cc2)nn2c1COCC2)N1CCC[C@H]1Cn1ccc(-c2ccc(F)cc2)n1] |
| 55 | 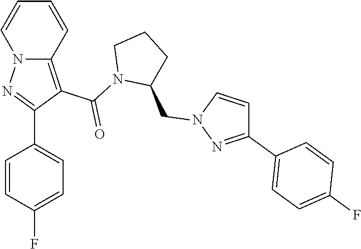 [SMILES: O=C(c1c(-c2ccc(F)cc2)nn2ccccc12)N1CCC[C@H]1Cn1ccc(-c2ccc(F)cc2)n1] |
| 56 | 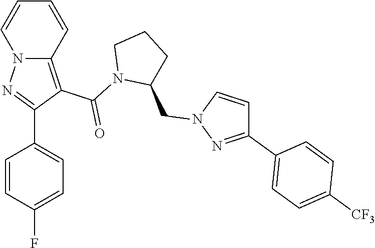 [SMILES: O=C(c1c(-c2ccc(F)cc2)nn2ccccc12)N1CCC[C@H]1Cn1ccc(-c2ccc(C(F)(F)F)cc2)n1] |
| 57 | 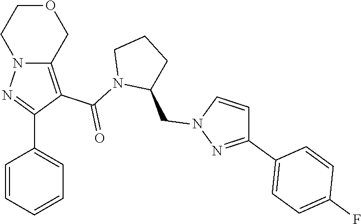 [SMILES: O=C(c1c(-c2ccccc2)nn2c1COCC2)N1CCC[C@H]1Cn1ccc(-c2ccc(F)cc2)n1] |
| 58 | 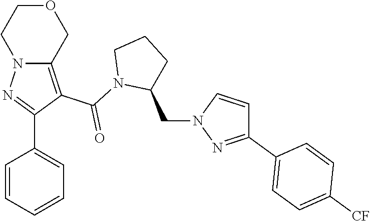 |
| 59 | 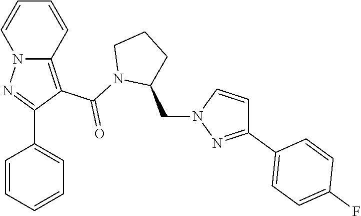 [SMILES: O=C(c1c(-c2ccccc2)nn2ccccc12)N1CCC[C@H]1Cn1ccc(-c2ccc(F)cc2)n1] |
| 60 | 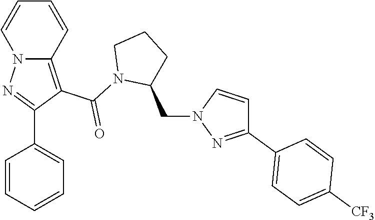 [SMILES: O=C(c1c(-c2ccccc2)nn2ccccc12)N1CCC[C@H]1Cn1ccc(-c2ccc(C(F)(F)F)cc2)n1] |
| 61 | 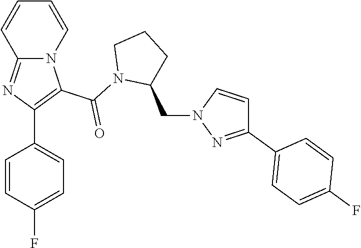 [SMILES: O=C(c1c(-c2ccc(F)cc2)nc2ccccn12)N1CCC[C@H]1Cn1ccc(-c2ccc(F)cc2)n1] |
| 62 | 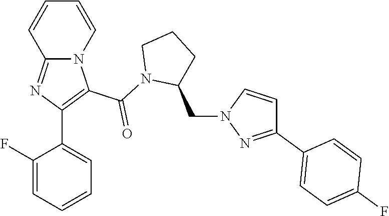 [SMILES: O=C(c1c(-c2ccccc2F)nc2ccccn12)N1CCC[C@H]1Cn1ccc(-c2ccc(F)cc2)n1] |
| 63 | 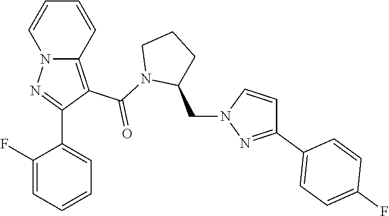 [SMILES: O=C(c1c(-c2ccccc2F)nn2ccccc12)N1CCC[C@H]1Cn1ccc(-c2ccc(F)cc2)n1] |
| 64 | 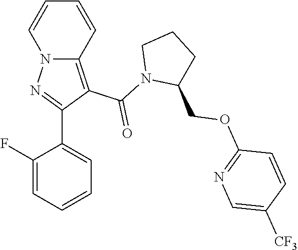 [SMILES: O=C(c1c(-c2ccccc2F)nn2ccccc12)N1CCC[C@H]1COc1ccc(C(F)(F)F)cn1] |
| 65 | 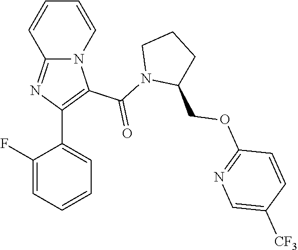 [SMILES: O=C(c1c(-c2ccccc2F)nc2ccccn12)N1CCC[C@H]1COc1ccc(C(F)(F)F)cn1] |
| 66 | 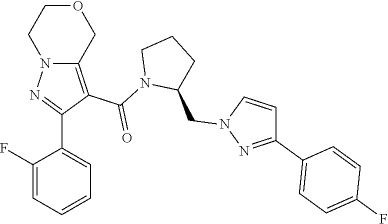 [SMILES: O=C(c1c(-c2ccccc2F)nn2c1COCC2)N1CCC[C@H]1Cn1ccc(-c2ccc(F)cc2)n1] |
| 67 | 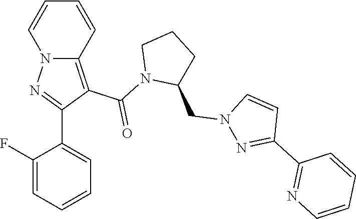 [SMILES: O=C(c1c(-c2ccccc2F)nn2ccccc12)N1CCC[C@H]1Cn1ccc(-c2ccccn2)n1] |
| 68 | 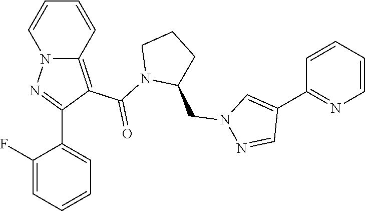 [SMILES: O=C(c1c(-c2ccccc2F)nn2ccccc12)N1CCC[C@H]1Cn1cc(-c2ccccn2)cn1] |
| 69 | 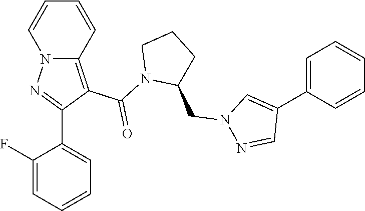 [SMILES: O=C(c1c(-c2ccccc2F)nn2ccccc12)N1CCC[C@H]1Cn1cc(-c2ccccc2)cn1] |
| 70 | 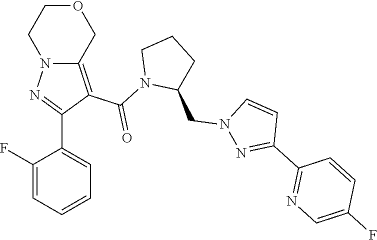 [SMILES: O=C(c1c(-c2ccccc2F)nn2c1COCC2)N1CCC[C@H]1Cn1ccc(-c2ccc(F)cn2)n1] |
| 71 | 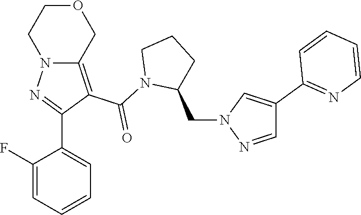 [SMILES: O=C(c1c(-c2ccccc2F)nn2c1COCC2)N1CCC[C@H]1Cn1cc(-c2ccccn2)cn1] |
| 72 | 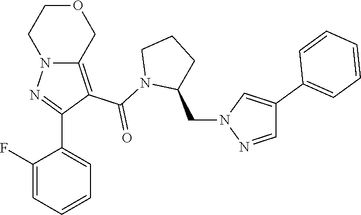 [SMILES: O=C(c1c(-c2ccccc2F)nn2c1COCC2)N1CCC[C@H]1Cn1cc(-c2ccccc2)cn1] |
| 73 | 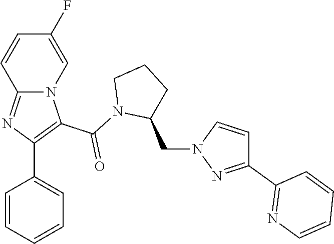 [SMILES: O=C(c1c(-c2ccccc2)nc2ccc(F)cn12)N1CCC[C@H]1Cn1ccc(-c2ccccn2)n1] |
| 74 | 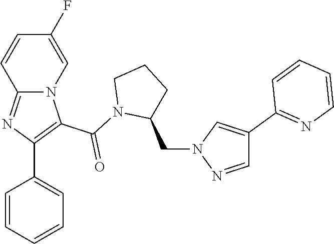 [SMILES: O=C(c1c(-c2ccccc2)nc2ccc(F)cn12)N1CCC[C@H]1Cn1cc(-c2ccccn2)cn1] |
| 75 | 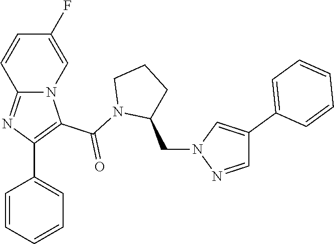 [SMILES: O=C(c1c(-c2ccccc2)nc2ccc(F)cn12)N1CCC[C@H]1Cn1cc(-c2ccccc2)cn1] |
| 76 | 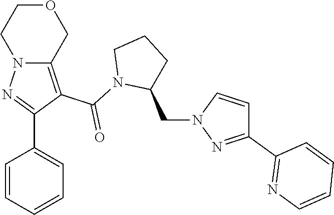 [SMILES: O=C(c1c(-c2ccccc2)nn2c1COCC2)N1CCC[C@H]1Cn1ccc(-c2ccccn2)n1] |
| 77 | 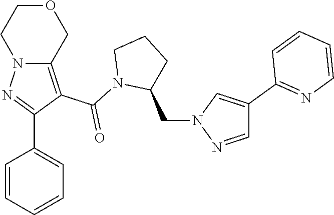 [SMILES: O=C(c1c(-c2ccccc2)nn2c1COCC2)N1CCC[C@H]1Cn1cc(-c2ccccn2)cn1] |
| 78 | 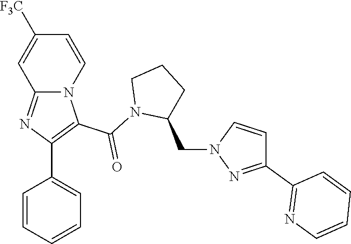 [SMILES: O=C(c1c(-c2ccccc2)nc2cc(C(F)(F)F)ccn12)N1CCC[C@H]1Cn1ccc(-c2ccccn2)n1] |
| 79 | 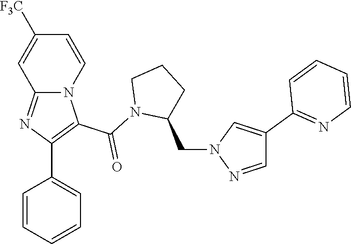 [SMILES: O=C(c1c(-c2ccccc2)nc2cc(C(F)(F)F)ccn12)N1CCC[C@H]1Cn1cc(-c2ccccn2)cn1] |
| 80 | 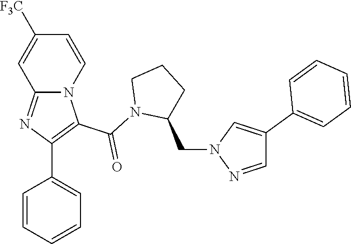 [SMILES: O=C(c1c(-c2ccccc2)nc2cc(C(F)(F)F)ccn12)N1CCC[C@H]1Cn1cc(-c2ccccc2)cn1] |
| 81 | 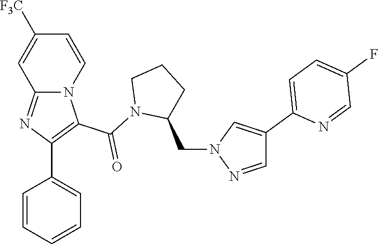 [SMILES: O=C(c1c(-c2ccccc2)nc2cc(C(F)(F)F)ccn12)N1CCC[C@H]1Cn1cc(-c2ccc(F)cn2)cn1] |
| 82 | 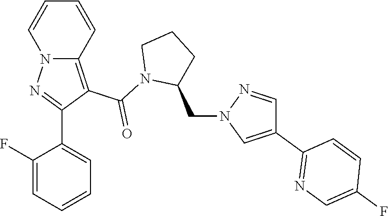 [SMILES: O=C(c1c(-c2ccccc2F)nn2ccccc12)N1CCC[C@H]1Cn1cc(-c2ccc(F)cn2)cn1] |
| 83 | 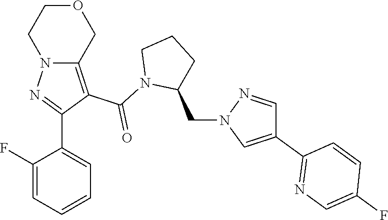 [SMILES: O=C(c1c(-c2ccccc2F)nn2c1COCC2)N1CCC[C@H]1Cn1cc(-c2ccc(F)cn2)cn1] |
| 84 | 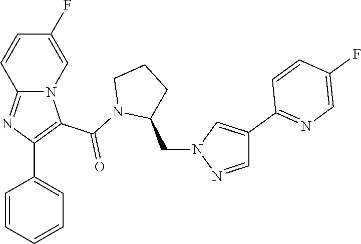 [SMILES: O=C(c1c(-c2ccccc2)nc2ccc(F)cn12)N1CCC[C@H]1Cn1cc(-c2ccc(F)cn2)cn1] |
| 85 | 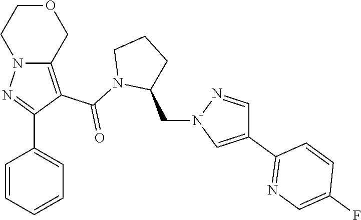 [SMILES: O=C(c1c(-c2ccccc2)nn2c1COCC2)N1CCC[C@H]1Cn1cc(-c2ccc(F)cn2)cn1] |
| 86 | 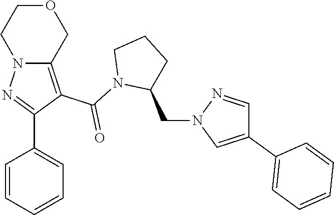 [SMILES: O=C(c1c(-c2ccccc2)nn2c1COCC2)N1CCC[C@H]1Cn1cc(-c2ccccc2)cn1] |
| 87 | 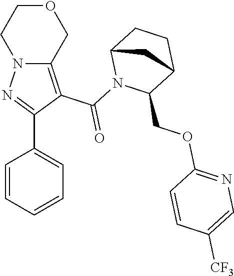 [SMILES: O=C(c1c(-c2ccccc2)nn2c1COCC2)N1[C@@H]2CC[C@@H](C2)[C@H]1COc1ccc(C(F)(F)F)cn1] |
| 88 | 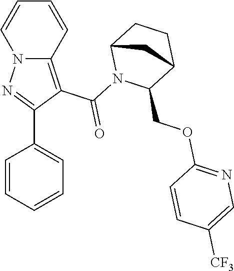 [SMILES: O=C(c1c(-c2ccccc2)nn2ccccc12)N1[C@@H]2CC[C@@H](C2)[C@H]1COc1ccc(C(F)(F)F)cn1] |
| 89 | 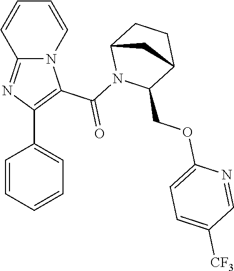 [SMILES: O=C(c1c(-c2ccccc2)nc2ccccn12)N1[C@@H]2CC[C@@H](C2)[C@H]1COc1ccc(C(F)(F)F)cn1] |
| 90 | 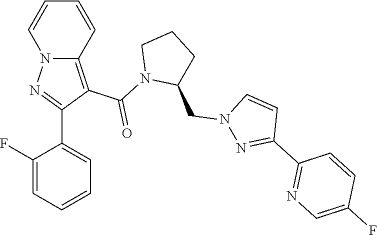 [SMILES: O=C(c1c(-c2ccccc2F)nn2ccccc12)N1CCC[C@H]1Cn1ccc(-c2ccc(F)cn2)n1] |
| 91 | 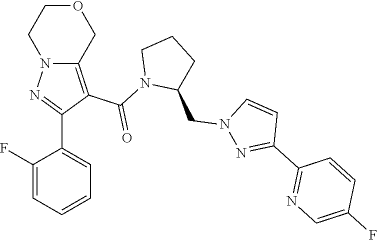 [SMILES: O=C(c1c(-c2ccccc2F)nn2c1COCC2)N1CCC[C@H]1Cn1ccc(-c2ccc(F)cn2)n1] |
| 92 | 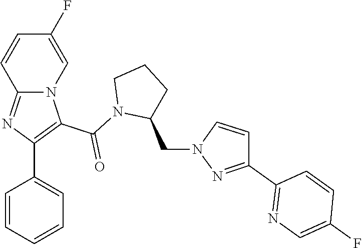 [SMILES: O=C(c1c(-c2ccccc2)nc2ccc(F)cn12)N1CCC[C@H]1Cn1ccc(-c2ccc(F)cn2)n1] |
| 93 | 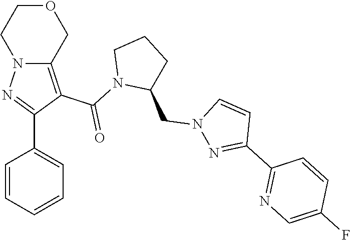 [SMILES: O=C(c1c(-c2ccccc2)nn2c1COCC2)N1CCC[C@H]1Cn1ccc(-c2ccc(F)cn2)n1] |
| 94 | 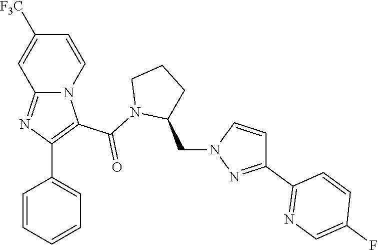 [SMILES: O=C(c1c(-c2ccccc2)nc2cc(C(F)(F)F)ccn12)N1CCC[C@H]1Cn1ccc(-c2ccc(F)cn2)n1] |
| 95 | 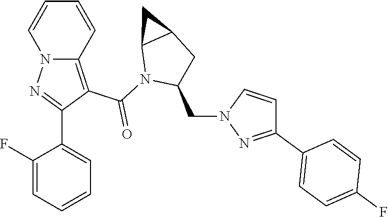 [SMILES: O=C(c1c(-c2ccccc2F)nn2ccccc12)N1[C@H](Cn2ccc(-c3ccc(F)cc3)n2)C[C@@H]2C[C@@H]21] |
| 96 | 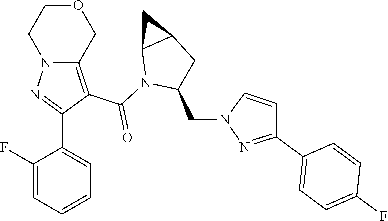 [SMILES: O=C(c1c(-c2ccccc2F)nn2c1COCC2)N1[C@H](Cn2ccc(-c3ccc(F)cc3)n2)C[C@@H]2C[C@@H]21] |
| 97 | 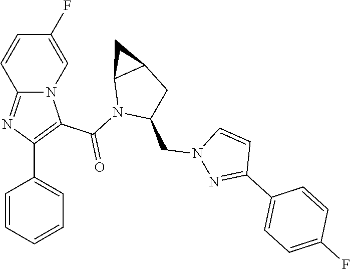 [SMILES: O=C(c1c(-c2ccccc2)nc2ccc(F)cn12)N1[C@H](Cn2ccc(-c3ccc(F)cc3)n2)C[C@@H]2C[C@@H]21] |
| 98 | 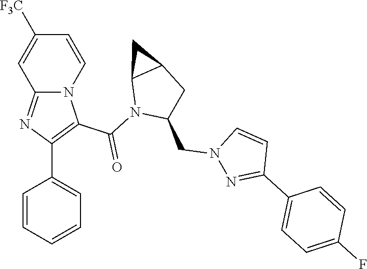 [SMILES: O=C(c1c(-c2ccccc2)nc2cc(C(F)(F)F)ccn12)N1[C@H](Cn2ccc(-c3ccc(F)cc3)n2)C[C@@H]2C[C@@H]21] |
| 99 | 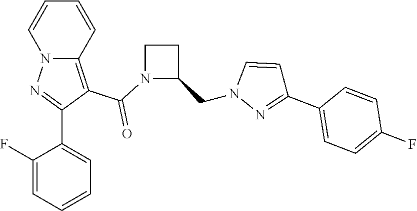 [SMILES: O=C(c1c(-c2ccccc2F)nn2ccccc12)N1CC[C@H]1Cn1ccc(-c2ccc(F)cc2)n1] |
| 100 | 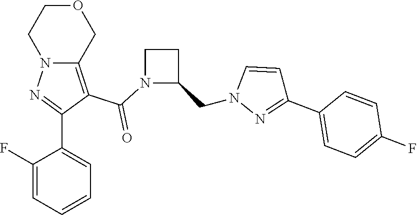 [SMILES: O=C(c1c(-c2ccccc2F)nn2c1COCC2)N1CC[C@H]1Cn1ccc(-c2ccc(F)cc2)n1] |
| 101 | 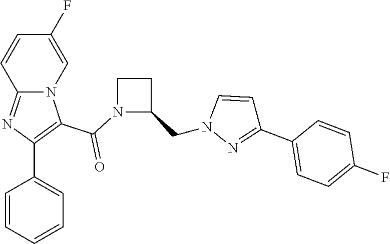 [SMILES: O=C(c1c(-c2ccccc2)nc2ccc(F)cn12)N1CC[C@H]1Cn1ccc(-c2ccc(F)cc2)n1] |
| 102 | 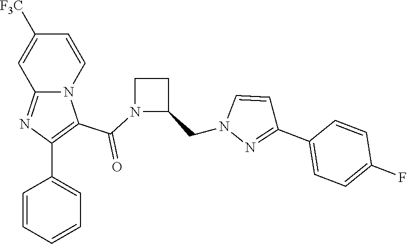 [SMILES: O=C(c1c(-c2ccccc2)nc2cc(C(F)(F)F)ccn12)N1CC[C@H]1Cn1ccc(-c2ccc(F)cc2)n1] |
| 103 | 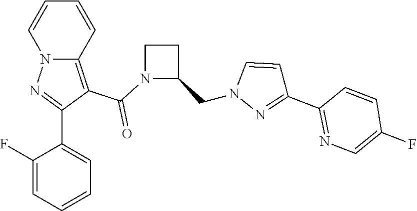 [SMILES: O=C(c1c(-c2ccccc2F)nn2ccccc12)N1CC[C@H]1Cn1ccc(-c2ccc(F)cn2)n1] |
| 104 | 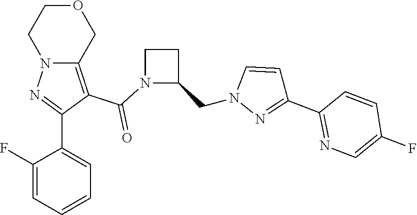 [SMILES: O=C(c1c(-c2ccccc2F)nn2c1COCC2)N1CC[C@H]1Cn1ccc(-c2ccc(F)cn2)n1] |
| 105 | 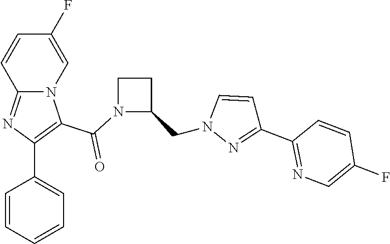 [SMILES: O=C(c1c(-c2ccccc2)nc2ccc(F)cn12)N1CC[C@H]1Cn1ccc(-c2ccc(F)cn2)n1] |
| 106 | 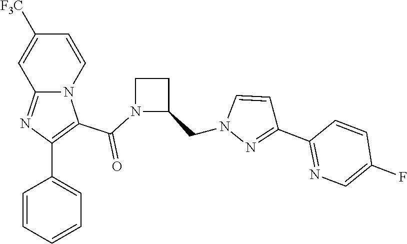 [SMILES: O=C(c1c(-c2ccccc2)nc2cc(C(F)(F)F)ccn12)N1CC[C@H]1Cn1ccc(-c2ccc(F)cn2)n1] |
| 107 | 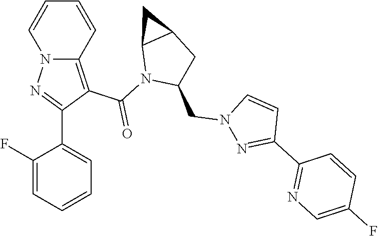 [SMILES: O=C(c1c(-c2ccccc2F)nn2ccccc12)N1[C@H](Cn2ccc(-c3ccc(F)cn3)n2)C[C@@H]2C[C@@H]21] |
| 108 | 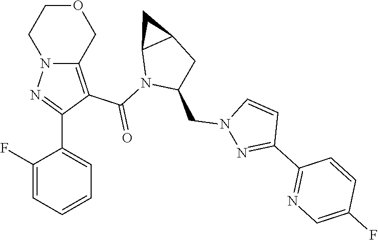 [SMILES: O=C(c1c(-c2ccccc2F)nn2c1COCC2)N1[C@H](Cn2ccc(-c3ccc(F)cn3)n2)C[C@@H]2C[C@@H]21] |
| 109 | 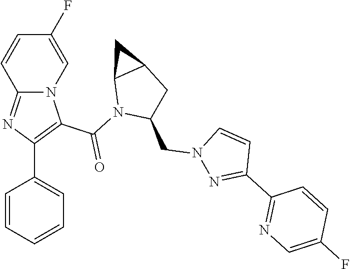 [SMILES: O=C(c1c(-c2ccccc2)nc2ccc(F)cn12)N1[C@H](Cn2ccc(-c3ccc(F)cn3)n2)C[C@@H]2C[C@@H]21] |
| 110 | 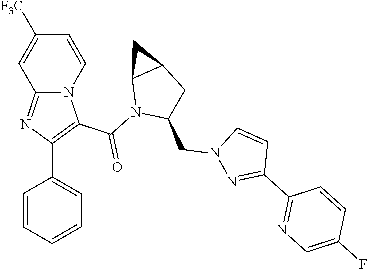 [SMILES: O=C(c1c(-c2ccccc2)nc2cc(C(F)(F)F)ccn12)N1[C@H](Cn2ccc(-c3ccc(F)cn3)n2)C[C@@H]2C[C@@H]21] |
| 111 | 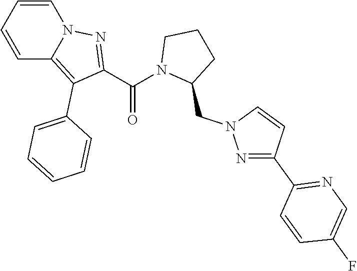 [SMILES: O=C(c1nn2ccccc2c1-c1ccccc1)N1CCC[C@H]1Cn1ccc(-c2ccc(F)cn2)n1] |
| 112 | 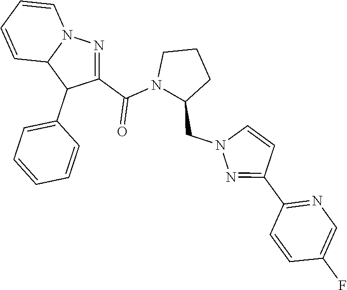 [SMILES: O=C(C1=NN2C=CC=CC2C1c1ccccc1)N1CCC[C@H]1Cn1ccc(-c2ccc(F)cn2)n1] |
| 113 | 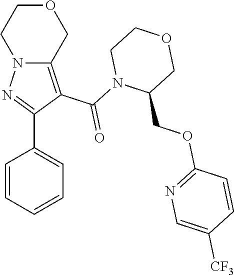 [SMILES: O=C(c1c(-c2ccccc2)nn2c1COCC2)N1CCOC[C@H]1COc1ccc(C(F)(F)F)cn1] |
| 114 | 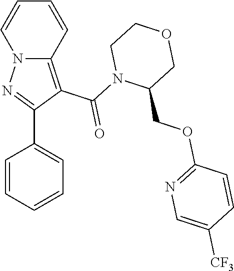 [SMILES: O=C(c1c(-c2ccccc2)nn2ccccc12)N1CCOC[C@H]1COc1ccc(C(F)(F)F)cn1] |
| 115 | 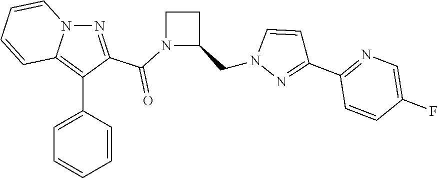 [SMILES: O=C(c1nn2ccccc2c1-c1ccccc1)N1CC[C@H]1Cn1ccc(-c2ccc(F)cn2)n1] |
| 116 | 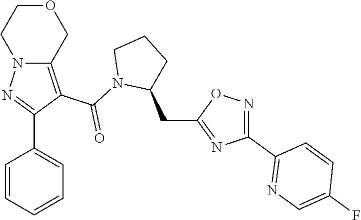 [SMILES: O=C(c1c(-c2ccccc2)nn2c1COCC2)N1CCC[C@H]1Cc1nc(-c2ccc(F)cn2)no1] |
| 117 | 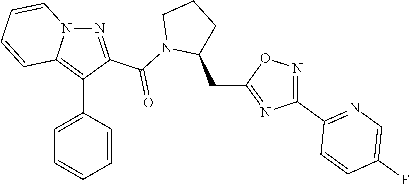 [SMILES: O=C(c1nn2ccccc2c1-c1ccccc1)N1CCC[C@H]1Cc1nc(-c2ccc(F)cn2)no1] |
| 118 | 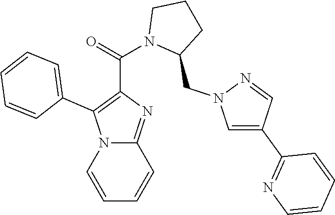 [SMILES: O=C(c1nc2ccccn2c1-c1ccccc1)N1CCC[C@H]1Cn1cc(-c2ccccn2)cn1] |
| 119 | 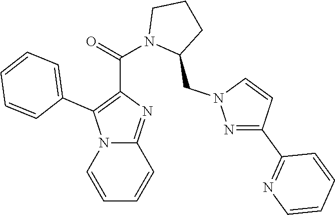 [SMILES: O=C(c1nc2ccccn2c1-c1ccccc1)N1CCC[C@H]1Cn1ccc(-c2ccccn2)n1] |
| 120 | 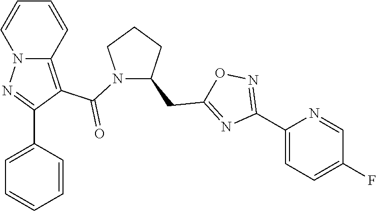 [SMILES: O=C(c1c(-c2ccccc2)nn2ccccc12)N1CCC[C@H]1Cc1nc(-c2ccc(F)cn2)no1] |
| 121 | 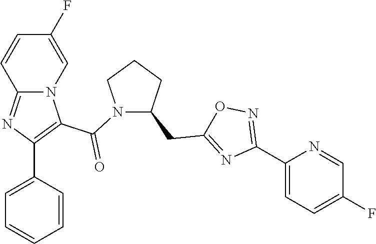 [SMILES: O=C(c1c(-c2ccccc2)nc2ccc(F)cn12)N1CCC[C@H]1Cc1nc(-c2ccc(F)cn2)no1] |
| 122 | 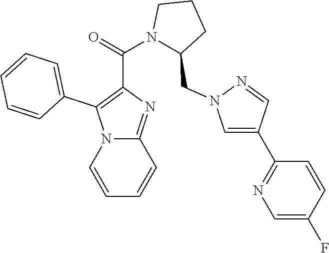 [SMILES: O=C(c1nc2ccccn2c1-c1ccccc1)N1CCC[C@H]1Cn1cc(-c2ccc(F)cn2)cn1] |
| 123 | 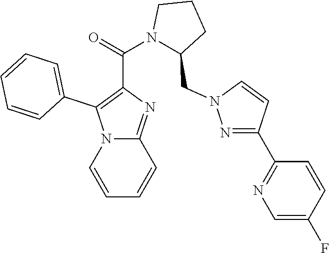 [SMILES: O=C(c1nc2ccccn2c1-c1ccccc1)N1CCC[C@H]1Cn1ccc(-c2ccc(F)cn2)n1] |
| 124 | 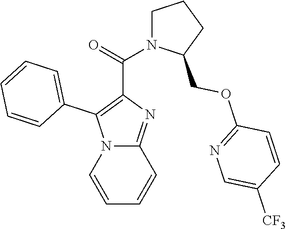 [SMILES: O=C(c1nc2ccccn2c1-c1ccccc1)N1CCC[C@H]1COc1ccc(C(F)(F)F)cn1] |
| 125 | 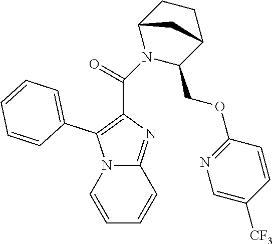 [SMILES: O=C(c1nc2ccccn2c1-c1ccccc1)N1[C@@H]2CC[C@@H](C2)[C@H]1COc1ccc(C(F)(F)F)cn1] |
| 126 | 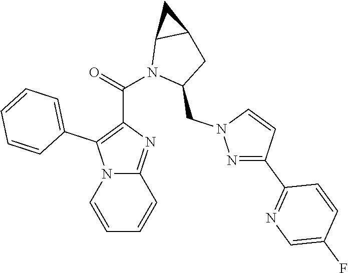 [SMILES: O=C(c1nc2ccccn2c1-c1ccccc1)N1[C@H](Cn2ccc(-c3ccc(F)cn3)n2)C[C@@H]2C[C@@H]21] |
| 127 | 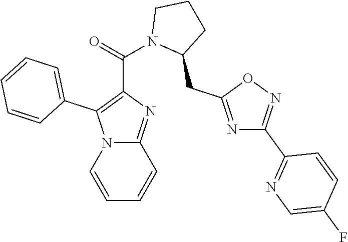 [SMILES: O=C(c1nc2ccccn2c1-c1ccccc1)N1CCC[C@H]1Cc1nc(-c2ccc(F)cn2)no1] |
| 128 | 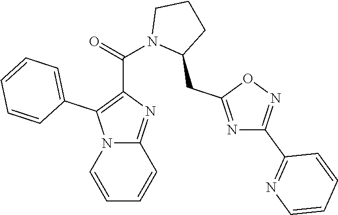 [SMILES: O=C(c1nc2ccccn2c1-c1ccccc1)N1CCC[C@H]1Cc1nc(-c2ccccn2)no1] |
| 129 | 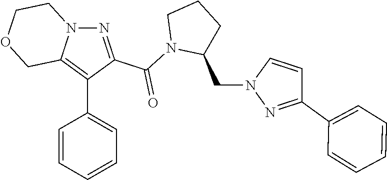 [SMILES: O=C(c1nn2c(c1-c1ccccc1)COCC2)N1CCC[C@H]1Cn1ccc(-c2ccccc2)n1] |
| 130 | 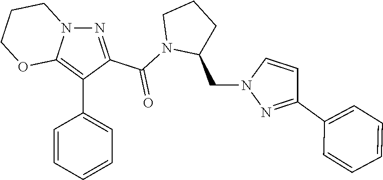 [SMILES: O=C(c1nn2c(c1-c1ccccc1)OCCC2)N1CCC[C@H]1Cn1ccc(-c2ccccc2)n1] |
| 131 | 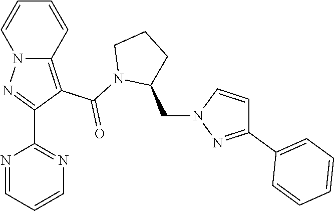 [SMILES: O=C(c1c(-c2ncccn2)nn2ccccc12)N1CCC[C@H]1Cn1ccc(-c2ccccc2)n1] |
| 132 | 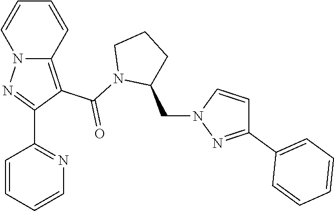 [SMILES: O=C(c1c(-c2ccccn2)nn2ccccc12)N1CCC[C@H]1Cn1ccc(-c2ccccc2)n1] |
| 133 | 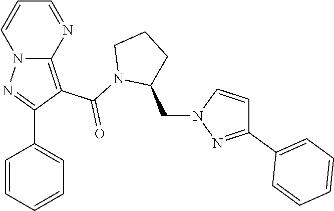 [SMILES: O=C(c1c(-c2ccccc2)nn2cccnc12)N1CCC[C@H]1Cn1ccc(-c2ccccc2)n1] |
| 134 | 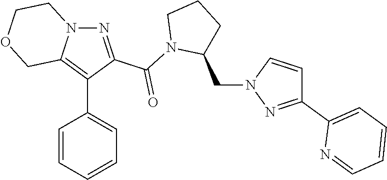 [SMILES: O=C(c1nn2c(c1-c1ccccc1)COCC2)N1CCC[C@H]1Cn1ccc(-c2ccccn2)n1] |
| 135 | 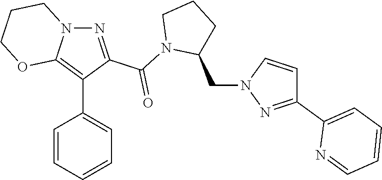 [SMILES: O=C(c1nn2c(c1-c1ccccc1)OCCC2)N1CCC[C@H]1Cn1ccc(-c2ccccn2)n1] |
| 136 | 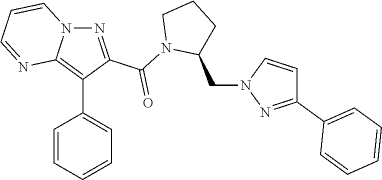 [SMILES: O=C(c1nn2cccnc2c1-c1ccccc1)N1CCC[C@H]1Cn1ccc(-c2ccccc2)n1] |
| 137 | 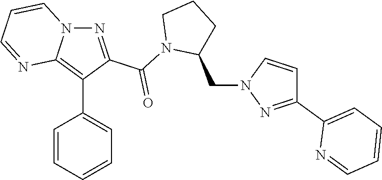 [SMILES: O=C(c1nn2cccnc2c1-c1ccccc1)N1CCC[C@H]1Cn1ccc(-c2ccccn2)n1] |
| 138 | 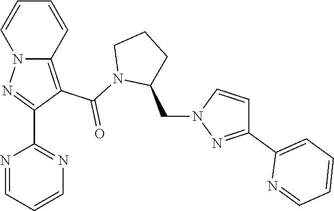 [SMILES: O=C(c1c(-c2ncccn2)nn2ccccc12)N1CCC[C@H]1Cn1ccc(-c2ccccn2)n1] |
| 139 | 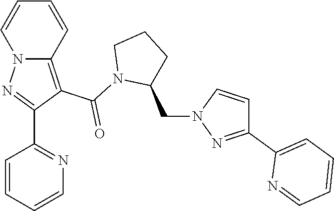 [SMILES: O=C(c1c(-c2ccccn2)nn2ccccc12)N1CCC[C@H]1Cn1ccc(-c2ccccn2)n1] |
| 140 | 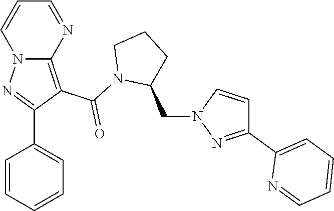 [SMILES: O=C(c1c(-c2ccccc2)nn2cccnc12)N1CCC[C@H]1Cn1ccc(-c2ccccn2)n1] |
| 141 | 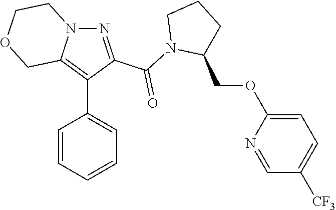 [SMILES: O=C(c1nn2c(c1-c1ccccc1)COCC2)N1CCC[C@H]1COc1ccc(C(F)(F)F)cn1] |
| 142 | 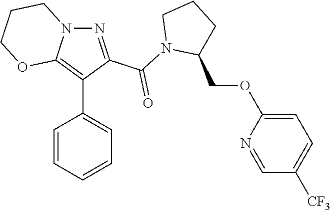 [SMILES: O=C(c1nn2c(c1-c1ccccc1)OCCC2)N1CCC[C@H]1COc1ccc(C(F)(F)F)cn1] |
| 143 | 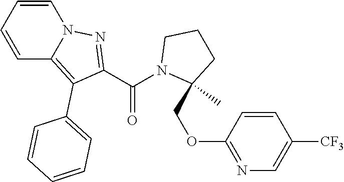 [SMILES: C[C@@]1(COc2ccc(C(F)(F)F)cn2)CCCN1C(=O)c1nn2ccccc2c1-c1ccccc1] |
| 144 | 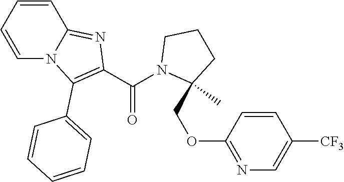 [SMILES: C[C@@]1(COc2ccc(C(F)(F)F)cn2)CCCN1C(=O)c1nc2ccccn2c1-c1ccccc1] |
| 145 | 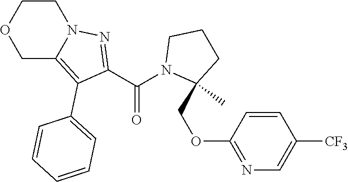 [SMILES: C[C@@]1(COc2ccc(C(F)(F)F)cn2)CCCN1C(=O)c1nn2c(c1-c1ccccc1)COCC2] |
| 146 | 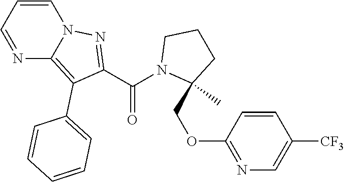 [SMILES: C[C@@]1(COc2ccc(C(F)(F)F)cn2)CCCN1C(=O)c1nn2cccnc2c1-c1ccccc1] |
| 147 | 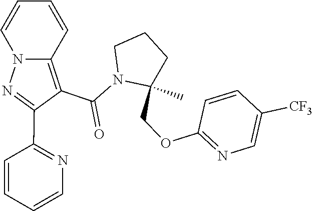 [SMILES: C[C@@]1(COc2ccc(C(F)(F)F)cn2)CCCN1C(=O)c1c(-c2ccccn2)nn2ccccc12] |
| 148 | 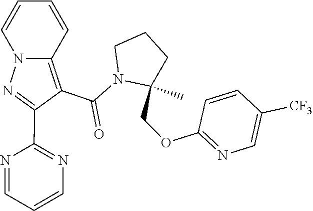 [SMILES: C[C@@]1(COc2ccc(C(F)(F)F)cn2)CCCN1C(=O)c1c(-c2ncccn2)nn2ccccc12] |
| 149 | 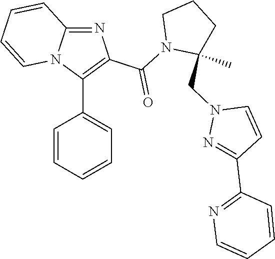 [SMILES: C[C@@]1(Cn2ccc(-c3ccccn3)n2)CCCN1C(=O)c1nc2ccccn2c1-c1ccccc1] |
| 150 | 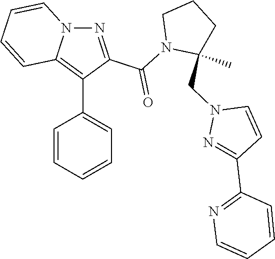 [SMILES: C[C@@]1(Cn2ccc(-c3ccccn3)n2)CCCN1C(=O)c1nn2ccccc2c1-c1ccccc1] |
| 151 | 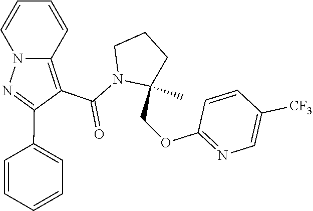 [SMILES: C[C@@]1(COc2ccc(C(F)(F)F)cn2)CCCN1C(=O)c1c(-c2ccccc2)nn2ccccc12] |
| 152 | 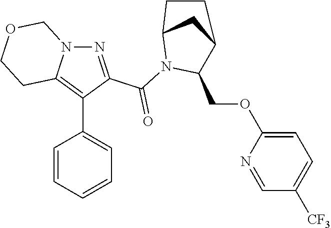 [SMILES: O=C(c1nn2c(c1-c1ccccc1)CCOC2)N1[C@@H]2CC[C@@H](C2)[C@H]1COc1ccc(C(F)(F)F)cn1] |
| 153 | 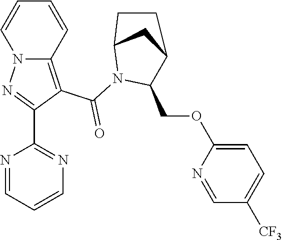 [SMILES: O=C(c1c(-c2ncccn2)nn2ccccc12)N1[C@@H]2CC[C@@H](C2)[C@H]1COc1ccc(C(F)(F)F)cn1] |
| 154 | 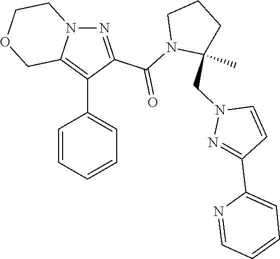 [SMILES: C[C@@]1(Cn2ccc(-c3ccccn3)n2)CCCN1C(=O)c1nn2c(c1-c1ccccc1)COCC2] |
| 155 | 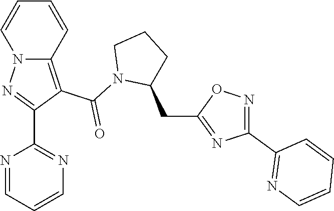 [SMILES: O=C(c1c(-c2ncccn2)nn2ccccc12)N1CCC[C@H]1Cc1nc(-c2ccccn2)no1] |
| 156 | 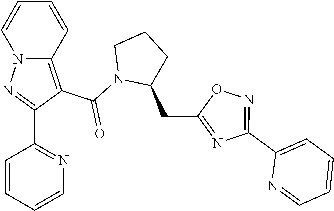 [SMILES: O=C(c1c(-c2ccccn2)nn2ccccc12)N1CCC[C@H]1Cc1nc(-c2ccccn2)no1] |
| 157 | 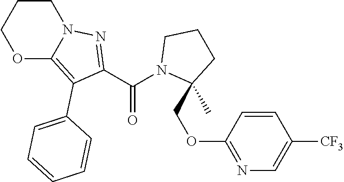 [SMILES: C[C@@]1(COc2ccc(C(F)(F)F)cn2)CCCN1C(=O)c1nn2c(c1-c1ccccc1)OCCC2] |
| 158 | 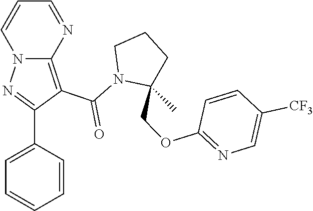 [SMILES: C[C@@]1(COc2ccc(C(F)(F)F)cn2)CCCN1C(=O)c1c(-c2ccccc2)nn2cccnc12] |
| 159 | 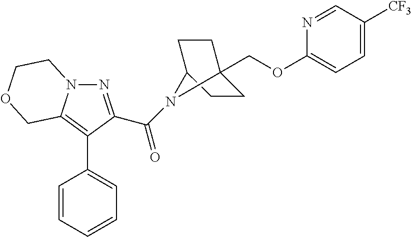 [SMILES: O=C(c1nn2c(c1-c1ccccc1)COCC2)N1C2CCC1(COc1ccc(C(F)(F)F)cn1)CC2] |
| 160 | 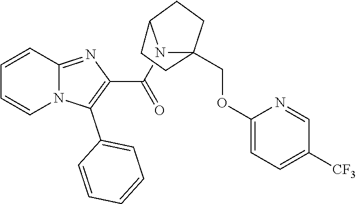 [SMILES: O=C(c1nc2ccccn2c1-c1ccccc1)N1C2CCC1(COc1ccc(C(F)(F)F)cn1)CC2] |
| 161 | 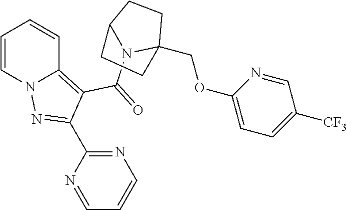 [SMILES: O=C(c1c(-c2ncccn2)nn2ccccc12)N1C2CCC1(COc1ccc(C(F)(F)F)cn1)CC2] |
| 162 | 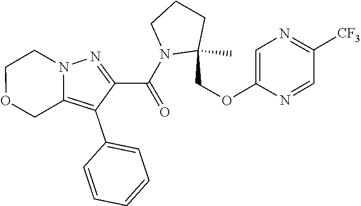 [SMILES: C[C@@]1(COc2cnc(C(F)(F)F)cn2)CCCN1C(=O)c1nn2c(c1-c1ccccc1)COCC2] |
| 163 | 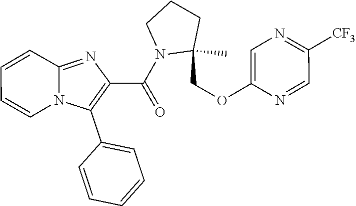 [SMILES: C[C@@]1(COc2cnc(C(F)(F)F)cn2)CCCN1C(=O)c1nc2ccccn2c1-c1ccccc1] |
| 164 | 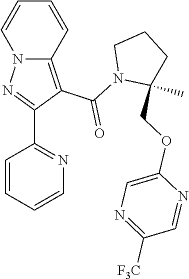 [SMILES: C[C@@]1(COc2cnc(C(F)(F)F)cn2)CCCN1C(=O)c1c(-c2ccccn2)nn2ccccc12] |
| 165 | 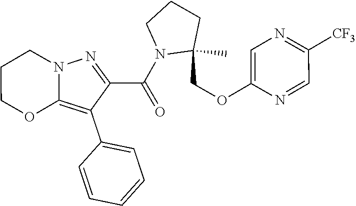 [SMILES: C[C@@]1(COc2cnc(C(F)(F)F)cn2)CCCN1C(=O)c1nn2c(c1-c1ccccc1)OCCC2] |
| 166 | 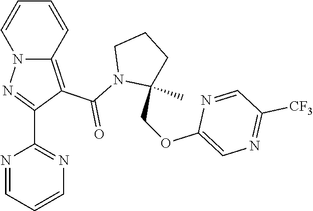 [SMILES: C[C@@]1(COc2cnc(C(F)(F)F)cn2)CCCN1C(=O)c1c(-c2ncccn2)nn2ccccc12] |
| 167 | 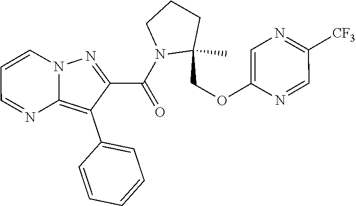 [SMILES: C[C@@]1(COc2cnc(C(F)(F)F)cn2)CCCN1C(=O)c1nn2cccnc2c1-c1ccccc1] |
| 168 | 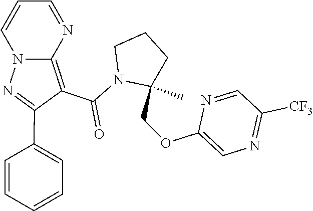 [SMILES: C[C@@]1(COc2cnc(C(F)(F)F)cn2)CCCN1C(=O)c1c(-c2ccccc2)nn2cccnc12] |
| 169 | 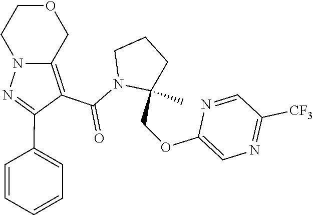 [SMILES: C[C@@]1(COc2cnc(C(F)(F)F)cn2)CCCN1C(=O)c1c(-c2ccccc2)nn2c1COCC2] |
| 170 | 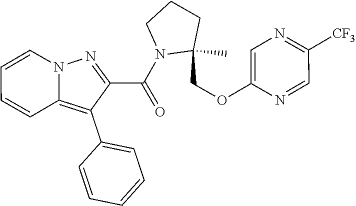 [SMILES: C[C@@]1(COc2cnc(C(F)(F)F)cn2)CCCN1C(=O)c1nn2ccccc2c1-c1ccccc1] |
| 171 | 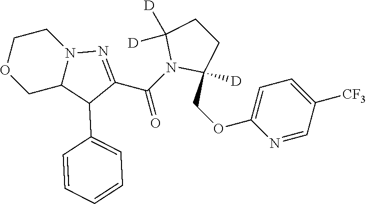 [SMILES: [2H]C1([2H])CC[C@@]([2H])(COc2ccc(C(F)(F)F)cn2)N1C(=O)C1=NN2CCOCC2C1c1ccccc1] |
| 172 | 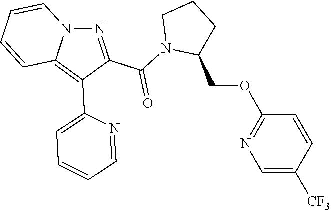 [SMILES: O=C(c1nn2ccccc2c1-c1ccccn1)N1CCC[C@H]1COc1ccc(C(F)(F)F)cn1] |
| 173 | 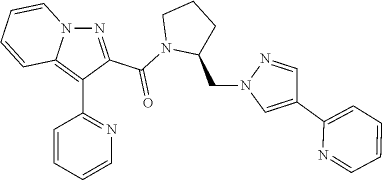 [SMILES: O=C(c1nn2ccccc2c1-c1ccccn1)N1CCC[C@H]1Cn1cc(-c2ccccn2)cn1] |
| 174 | 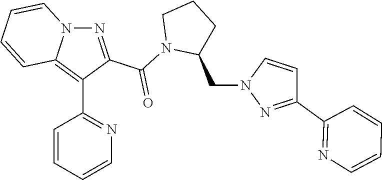 [SMILES: O=C(c1nn2ccccc2c1-c1ccccn1)N1CCC[C@H]1Cn1ccc(-c2ccccn2)n1] |
| 175 | 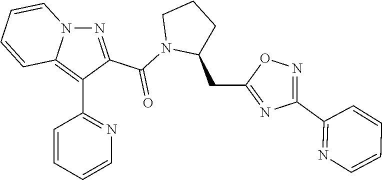 [SMILES: O=C(c1nn2ccccc2c1-c1ccccn1)N1CCC[C@H]1Cc1nc(-c2ccccn2)no1] |
| 176 | 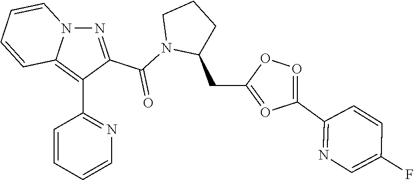 |
| 177 | 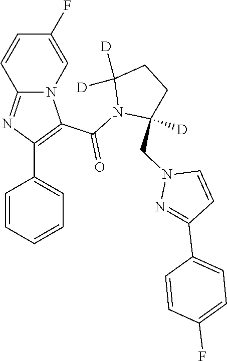 [SMILES: [2H]C1([2H])CC[C@@]([2H])(Cn2ccc(-c3ccc(F)cc3)n2)N1C(=O)c1c(-c2ccccc2)nc2ccc(F)cn12] |
| 178 | 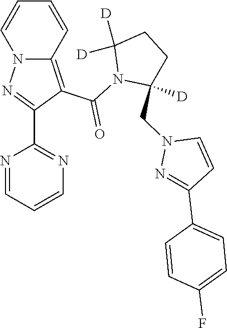 [SMILES: [2H]C1([2H])CC[C@@]([2H])(Cn2ccc(-c3ccc(F)cc3)n2)N1C(=O)c1c(-c2ncccn2)nn2ccccc12] |
| 179 | 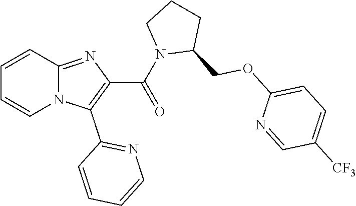 [SMILES: O=C(c1nc2ccccn2c1-c1ccccn1)N1CCC[C@H]1COc1ccc(C(F)(F)F)cn1] |
| 180 | 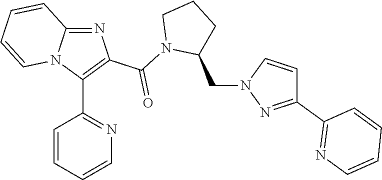 [SMILES: O=C(c1nc2ccccn2c1-c1ccccn1)N1CCC[C@H]1Cn1ccc(-c2ccccn2)n1] |
| 181 | 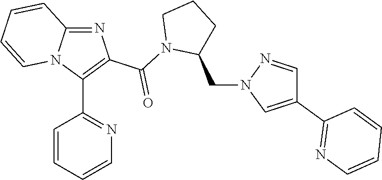 [SMILES: O=C(c1nc2ccccn2c1-c1ccccn1)N1CCC[C@H]1Cn1cc(-c2ccccn2)cn1] |
| 182 | 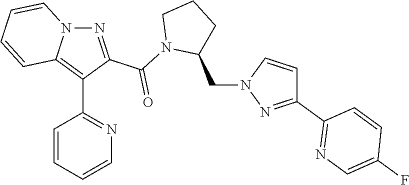 [SMILES: O=C(c1nn2ccccc2c1-c1ccccn1)N1CCC[C@H]1Cn1ccc(-c2ccc(F)cn2)n1] |
| 183 | 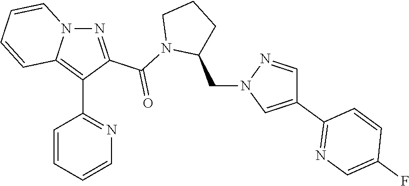 [SMILES: O=C(c1nn2ccccc2c1-c1ccccn1)N1CCC[C@H]1Cn1cc(-c2ccc(F)cn2)cn1] |
| 184 | 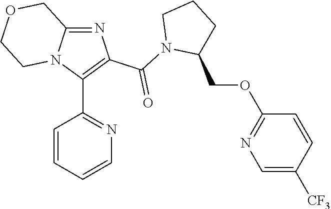 [SMILES: O=C(c1nc2n(c1-c1ccccn1)CCOC2)N1CCC[C@H]1COc1ccc(C(F)(F)F)cn1] |
| 185 | 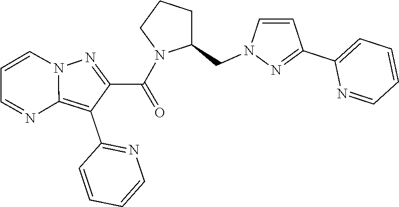 [SMILES: O=C(c1nn2cccnc2c1-c1ccccn1)N1CCC[C@H]1Cn1ccc(-c2ccccn2)n1] |
| 186 | 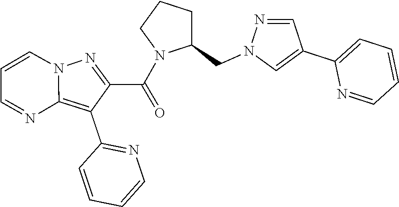 [SMILES: O=C(c1nn2cccnc2c1-c1ccccn1)N1CCC[C@H]1Cn1cc(-c2ccccn2)cn1] |
| 187 | 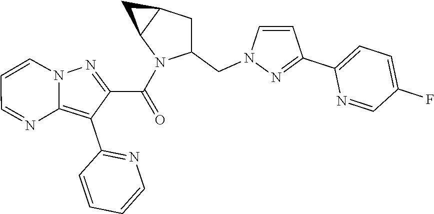 [SMILES: O=C(c1nn2cccnc2c1-c1ccccn1)N1C(Cn2ccc(-c3ccc(F)cn3)n2)C[C@@H]2C[C@@H]21] |
| 188 | 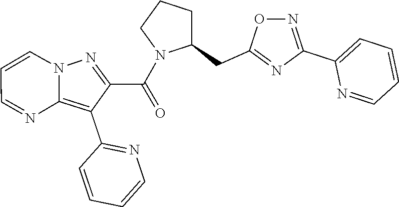 [SMILES: O=C(c1nn2cccnc2c1-c1ccccn1)N1CCC[C@H]1Cc1nc(-c2ccccn2)no1] |
| 189 | 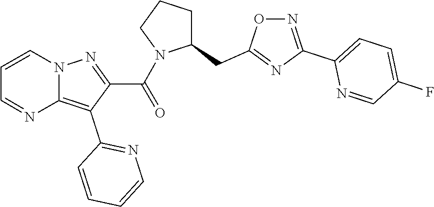 [SMILES: O=C(c1nn2cccnc2c1-c1ccccn1)N1CCC[C@H]1Cc1nc(-c2ccc(F)cn2)no1] |
| 190 | 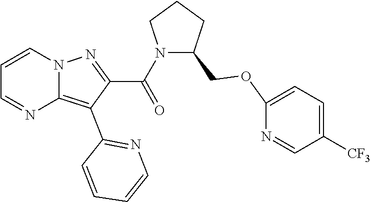 [SMILES: O=C(c1nn2cccnc2c1-c1ccccn1)N1CCC[C@H]1COc1ccc(C(F)(F)F)cn1] |
| 191 | 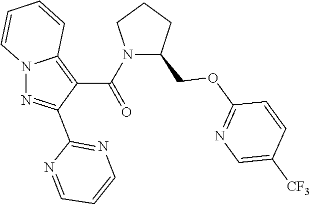 [SMILES: O=C(c1c(-c2ncccn2)nn2ccccc12)N1CCC[C@H]1COc1ccc(C(F)(F)F)cn1] |
| 192 | 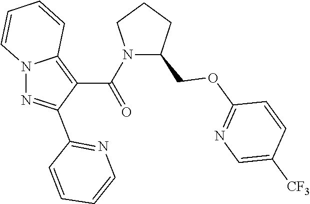 [SMILES: O=C(c1c(-c2ccccn2)nn2ccccc12)N1CCC[C@H]1COc1ccc(C(F)(F)F)cn1] |
| 193 | 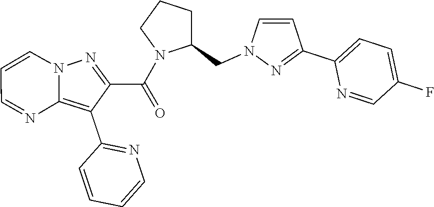 [SMILES: O=C(c1nn2cccnc2c1-c1ccccn1)N1CCC[C@H]1Cn1ccc(-c2ccc(F)cn2)n1] |
| 194 | 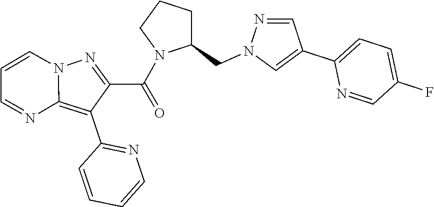 [SMILES: O=C(c1nn2cccnc2c1-c1ccccn1)N1CCC[C@H]1Cn1cc(-c2ccc(F)cn2)cn1] |
| 195 | 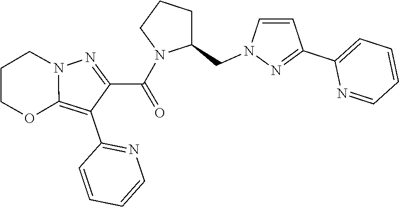 [SMILES: O=C(c1nn2c(c1-c1ccccn1)OCCC2)N1CCC[C@H]1Cn1ccc(-c2ccccn2)n1] |
| 196 | 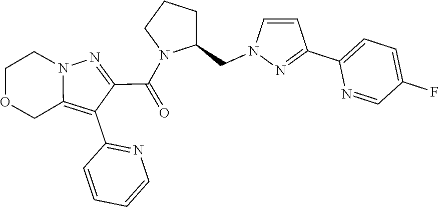 [SMILES: O=C(c1nn2c(c1-c1ccccn1)COCC2)N1CCC[C@H]1Cn1ccc(-c2ccc(F)cn2)n1] |
| 197 | 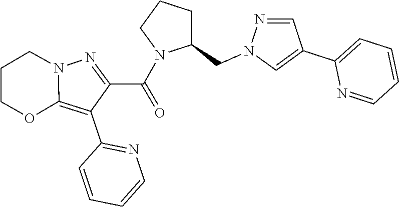 [SMILES: O=C(c1nn2c(c1-c1ccccn1)OCCC2)N1CCC[C@H]1Cn1cc(-c2ccccn2)cn1] |
| 198 | 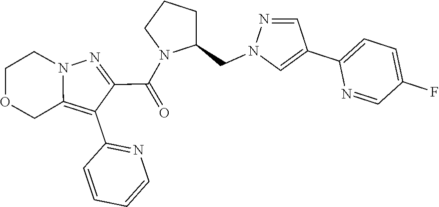 [SMILES: O=C(c1nn2c(c1-c1ccccn1)COCC2)N1CCC[C@H]1Cn1cc(-c2ccc(F)cn2)cn1] |
| 199 | 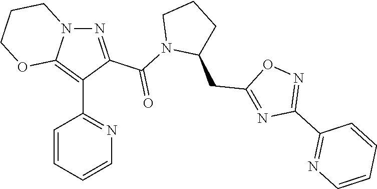 [SMILES: O=C(c1nn2c(c1-c1ccccn1)OCCC2)N1CCC[C@H]1Cc1nc(-c2ccccn2)no1] |
| 200 | 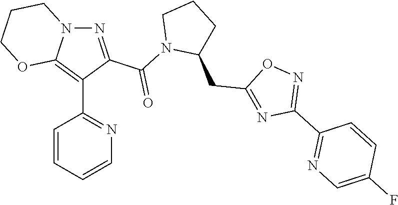 [SMILES: O=C(c1nn2c(c1-c1ccccn1)OCCC2)N1CCC[C@H]1Cc1nc(-c2ccc(F)cn2)no1] |
| 201 | 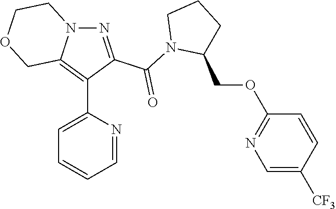 [SMILES: O=C(c1nn2c(c1-c1ccccn1)COCC2)N1CCC[C@H]1COc1ccc(C(F)(F)F)cn1] |
| 202 | 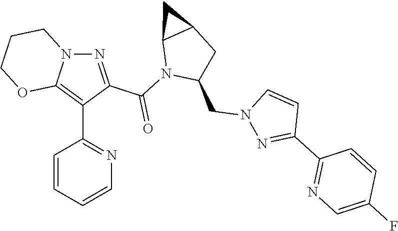 [SMILES: O=C(c1nn2c(c1-c1ccccn1)OCCC2)N1[C@H](Cn2ccc(-c3ccc(F)cn3)n2)C[C@@H]2C[C@@H]21] |
| 203 | 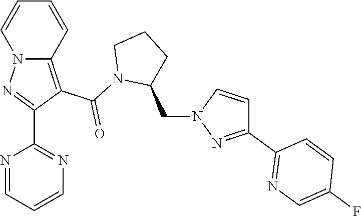 [SMILES: O=C(c1c(-c2ncccn2)nn2ccccc12)N1CCC[C@H]1Cn1ccc(-c2ccc(F)cn2)n1] |
| 204 | 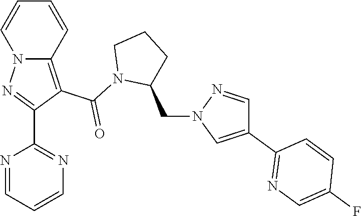 [SMILES: O=C(c1c(-c2ncccn2)nn2ccccc12)N1CCC[C@H]1Cn1cc(-c2ccc(F)cn2)cn1] |
| 205 | 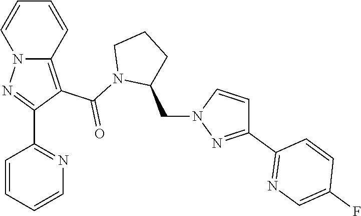 [SMILES: O=C(c1c(-c2ccccn2)nn2ccccc12)N1CCC[C@H]1Cn1ccc(-c2ccc(F)cn2)n1] |
| 206 | 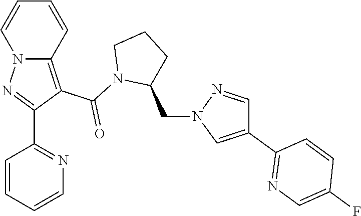 [SMILES: O=C(c1c(-c2ccccn2)nn2ccccc12)N1CCC[C@H]1Cn1cc(-c2ccc(F)cn2)cn1] |
| 207 | 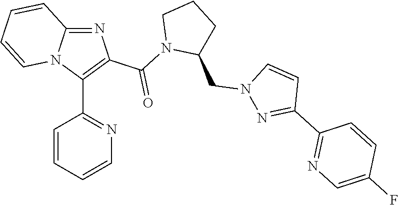 [SMILES: O=C(c1nc2ccccn2c1-c1ccccn1)N1CCC[C@H]1Cn1ccc(-c2ccc(F)cn2)n1] |
| 208 | 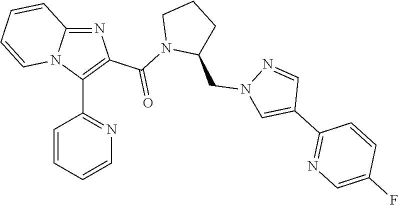 [SMILES: O=C(c1nc2ccccn2c1-c1ccccn1)N1CCC[C@H]1Cn1cc(-c2ccc(F)cn2)cn1] |
| 209 | 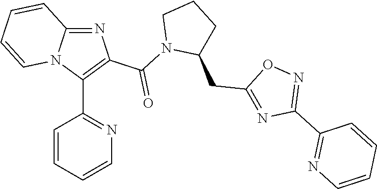 [SMILES: O=C(c1nc2ccccn2c1-c1ccccn1)N1CCC[C@H]1Cc1nc(-c2ccccn2)no1] |
| 210 | 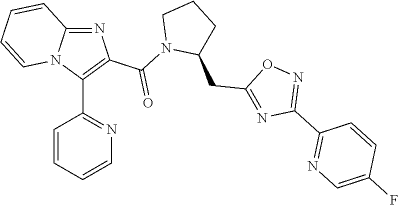 [SMILES: O=C(c1nc2ccccn2c1-c1ccccn1)N1CCC[C@H]1Cc1nc(-c2ccc(F)cn2)no1] |
| 211 | 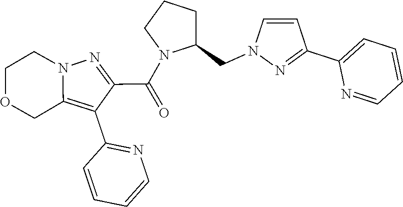 [SMILES: O=C(c1nn2c(c1-c1ccccn1)COCC2)N1CCC[C@H]1Cn1ccc(-c2ccccn2)n1] |
| 212 | 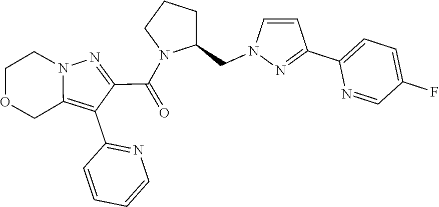 [SMILES: O=C(c1nn2c(c1-c1ccccn1)COCC2)N1CCC[C@H]1Cn1ccc(-c2ccc(F)cn2)n1] |
| 213 | 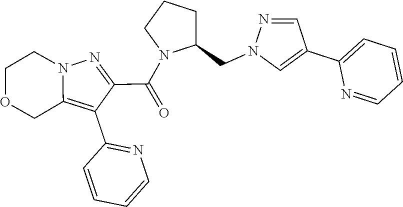 [SMILES: O=C(c1nn2c(c1-c1ccccn1)COCC2)N1CCC[C@H]1Cn1cc(-c2ccccn2)cn1] |
| 214 | 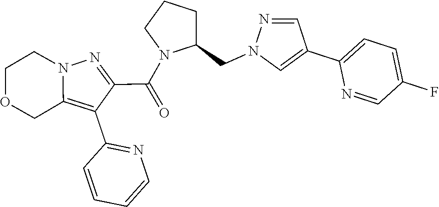 [SMILES: O=C(c1nn2c(c1-c1ccccn1)COCC2)N1CCC[C@H]1Cn1cc(-c2ccc(F)cn2)cn1] |
| 215 | 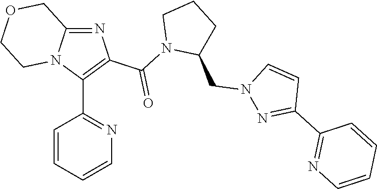 [SMILES: O=C(c1nc2n(c1-c1ccccn1)CCOC2)N1CCC[C@H]1Cn1ccc(-c2ccccn2)n1] |
| 216 | 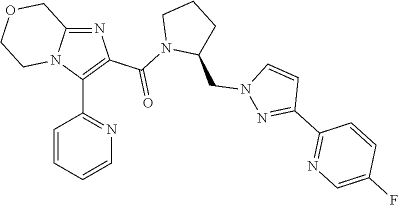 [SMILES: O=C(c1nc2n(c1-c1ccccn1)CCOC2)N1CCC[C@H]1Cn1ccc(-c2ccc(F)cn2)n1] |
| 217 | 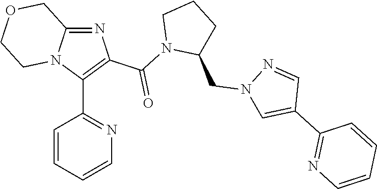 [SMILES: O=C(c1nc2n(c1-c1ccccn1)CCOC2)N1CCC[C@H]1Cn1cc(-c2ccccn2)cn1] |
| 218 | 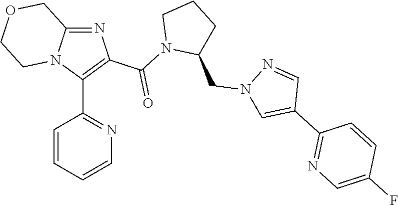 [SMILES: O=C(c1nc2n(c1-c1ccccn1)CCOC2)N1CCC[C@H]1Cn1cc(-c2ccc(F)cn2)cn1] |
| 219 | 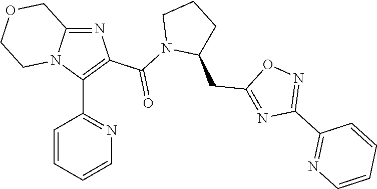 [SMILES: O=C(c1nc2n(c1-c1ccccn1)CCOC2)N1CCC[C@H]1Cc1nc(-c2ccccn2)no1] |
| 220 | 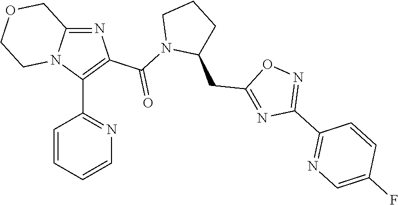 [SMILES: O=C(c1nc2n(c1-c1ccccn1)CCOC2)N1CCC[C@H]1Cc1nc(-c2ccc(F)cn2)no1] |
| 221 | 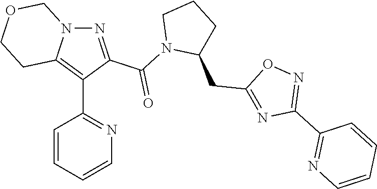 [SMILES: O=C(c1nn2c(c1-c1ccccn1)CCOC2)N1CCC[C@H]1Cc1nc(-c2ccccn2)no1] |
| 222 | 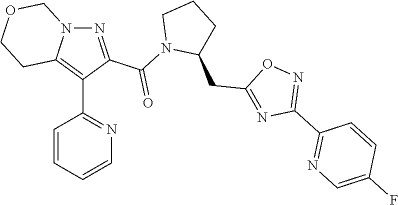 [SMILES: O=C(c1nn2c(c1-c1ccccn1)CCOC2)N1CCC[C@H]1Cc1nc(-c2ccc(F)cn2)no1] |
| 223 | 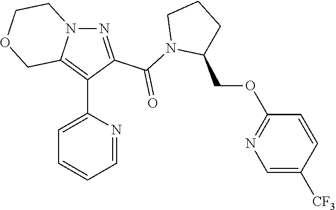 [SMILES: O=C(c1nn2c(c1-c1ccccn1)COCC2)N1CCC[C@H]1COc1ccc(C(F)(F)F)cn1] |
| 224 | 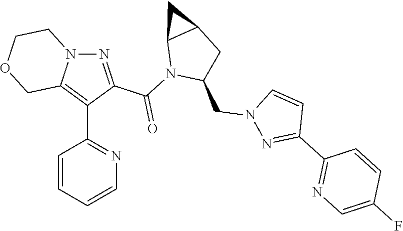 [SMILES: O=C(c1nn2c(c1-c1ccccn1)COCC2)N1[C@H](Cn2ccc(-c3ccc(F)cn3)n2)C[C@@H]2C[C@@H]21] |
| 225 | 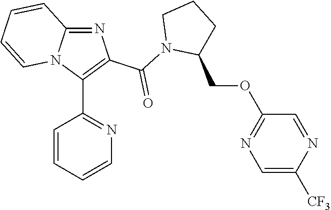 [SMILES: O=C(c1nc2ccccn2c1-c1ccccn1)N1CCC[C@H]1COc1cnc(C(F)(F)F)cn1] |
| 226 | 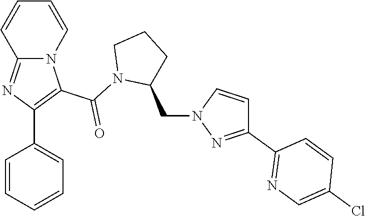 [SMILES: O=C(c1c(-c2ccccc2)nc2ccccn12)N1CCC[C@H]1Cn1ccc(-c2ccc(Cl)cn2)n1] |
| 227 | 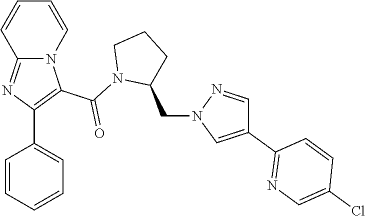 [SMILES: O=C(c1c(-c2ccccc2)nc2ccccn12)N1CCC[C@H]1Cn1cc(-c2ccc(Cl)cn2)cn1] |
| 228 | 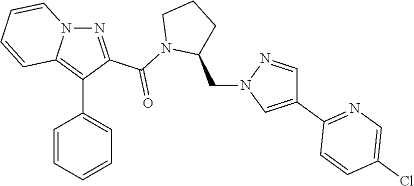 [SMILES: O=C(c1nn2ccccc2c1-c1ccccc1)N1CCC[C@H]1Cn1cc(-c2ccc(Cl)cn2)cn1] |
| 229 | 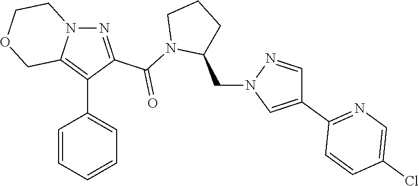 [SMILES: O=C(c1nn2c(c1-c1ccccc1)COCC2)N1CCC[C@H]1Cn1cc(-c2ccc(Cl)cn2)cn1] |
| 230 | 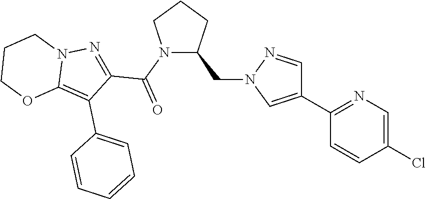 [SMILES: O=C(c1nn2c(c1-c1ccccc1)OCCC2)N1CCC[C@H]1Cn1cc(-c2ccc(Cl)cn2)cn1] |
| 231 | 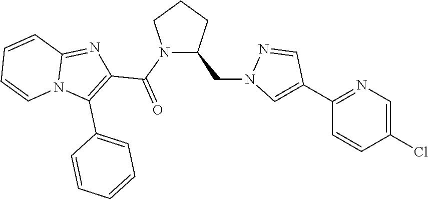 [SMILES: O=C(c1nc2ccccn2c1-c1ccccc1)N1CCC[C@H]1Cn1cc(-c2ccc(Cl)cn2)cn1] |
| 232 | 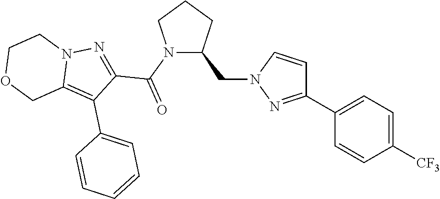 [SMILES: O=C(c1nn2c(c1-c1ccccc1)COCC2)N1CCC[C@H]1Cn1ccc(-c2ccc(C(F)(F)F)cc2)n1] |
| 234 | 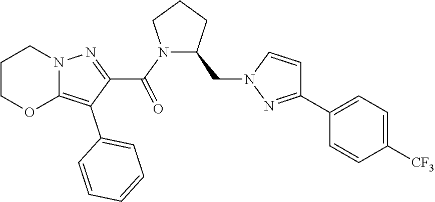 [SMILES: O=C(c1nn2c(c1-c1ccccc1)OCCC2)N1CCC[C@H]1Cn1ccc(-c2ccc(C(F)(F)F)cc2)n1] |
| 235 | 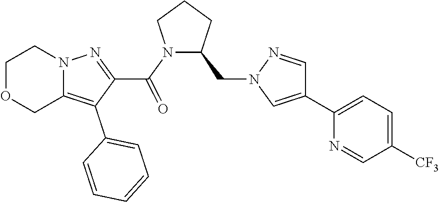 [SMILES: O=C(c1nn2c(c1-c1ccccc1)COCC2)N1CCC[C@H]1Cn1cc(-c2ccc(C(F)(F)F)cn2)cn1] |
| 236 | 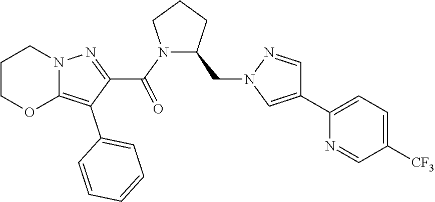 [SMILES: O=C(c1nn2c(c1-c1ccccc1)OCCC2)N1CCC[C@H]1Cn1cc(-c2ccc(C(F)(F)F)cn2)cn1] |
| 237 | 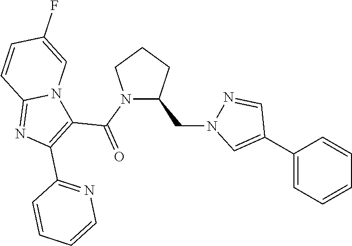 [SMILES: O=C(c1c(-c2ccccn2)nc2ccc(F)cn12)N1CCC[C@H]1Cn1cc(-c2ccccc2)cn1] |
| 238 | 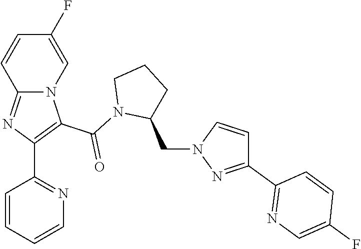 [SMILES: O=C(c1c(-c2ccccn2)nc2ccc(F)cn12)N1CCC[C@H]1Cn1ccc(-c2ccc(F)cn2)n1] |
| 239 | 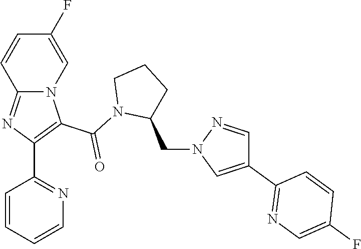 [SMILES: O=C(c1c(-c2ccccn2)nc2ccc(F)cn12)N1CCC[C@H]1Cn1cc(-c2ccc(F)cn2)cn1] |
| 240 | 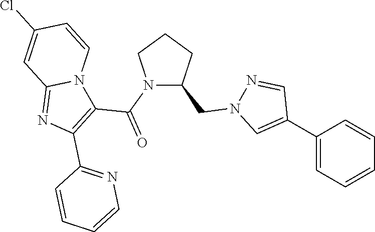 [SMILES: O=C(c1c(-c2ccccn2)nc2cc(Cl)ccn12)N1CCC[C@H]1Cn1cc(-c2ccccc2)cn1] |
| 241 | 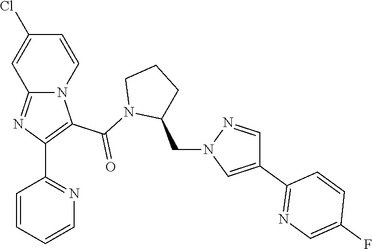 [SMILES: O=C(c1c(-c2ccccn2)nc2cc(Cl)ccn12)N1CCC[C@H]1Cn1cc(-c2ccc(F)cn2)cn1] |
| 242 | 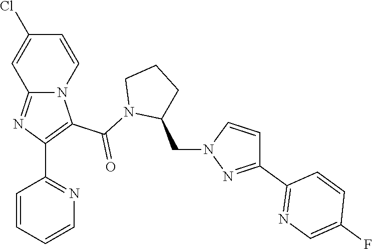 [SMILES: O=C(c1c(-c2ccccn2)nc2cc(Cl)ccn12)N1CCC[C@H]1Cn1ccc(-c2ccc(F)cn2)n1] |
| 243 | 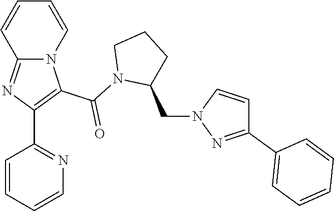 [SMILES: O=C(c1c(-c2ccccn2)nc2ccccn12)N1CCC[C@H]1Cn1ccc(-c2ccccc2)n1] |
| 244 | 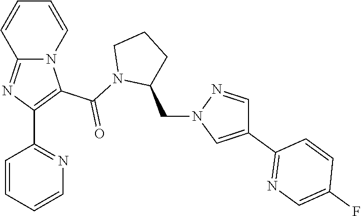 [SMILES: O=C(c1c(-c2ccccn2)nc2ccccn12)N1CCC[C@H]1Cn1cc(-c2ccc(F)cn2)cn1] |
| 245 | 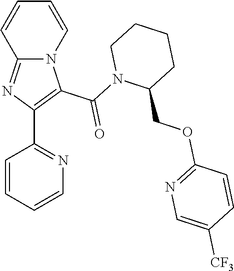 [SMILES: O=C(c1c(-c2ccccn2)nc2ccccn12)N1CCCC[C@H]1COc1ccc(C(F)(F)F)cn1] |
| 246 | 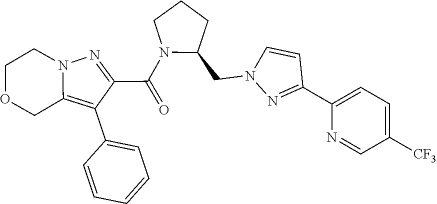 [SMILES: O=C(c1nn2c(c1-c1ccccc1)COCC2)N1CCC[C@H]1Cn1ccc(-c2ccc(C(F)(F)F)cn2)n1] |
| 247 | 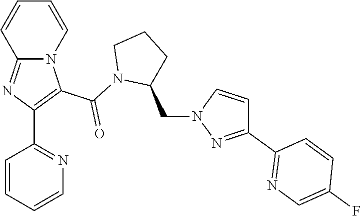 [SMILES: O=C(c1c(-c2ccccn2)nc2ccccn12)N1CCC[C@H]1Cn1ccc(-c2ccc(F)cn2)n1] |
| 248 | 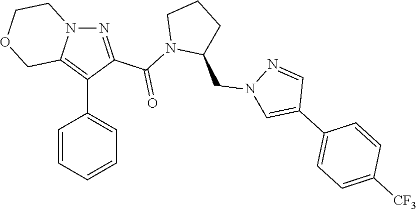 [SMILES: O=C(c1nn2c(c1-c1ccccc1)COCC2)N1CCC[C@H]1Cn1cc(-c2ccc(C(F)(F)F)cc2)cn1] |
| 249 | 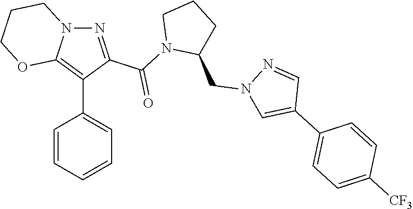 [SMILES: O=C(c1nn2c(c1-c1ccccc1)OCCC2)N1CCC[C@H]1Cn1cc(-c2ccc(C(F)(F)F)cc2)cn1] |
| 250 | 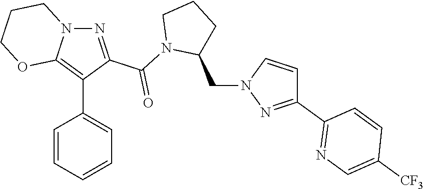 [SMILES: O=C(c1nn2c(c1-c1ccccc1)OCCC2)N1CCC[C@H]1Cn1ccc(-c2ccc(C(F)(F)F)cn2)n1] |
| 251 | 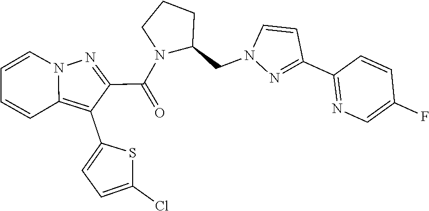 [SMILES: O=C(c1nn2ccccc2c1-c1ccc(Cl)s1)N1CCC[C@H]1Cn1ccc(-c2ccc(F)cn2)n1] |
| 252 | 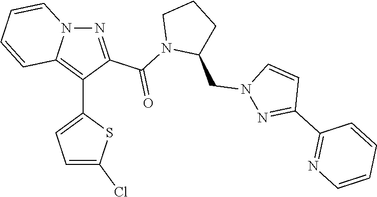 [SMILES: O=C(c1nn2ccccc2c1-c1ccc(Cl)s1)N1CCC[C@H]1Cn1ccc(-c2ccccn2)n1] |
| 253 | 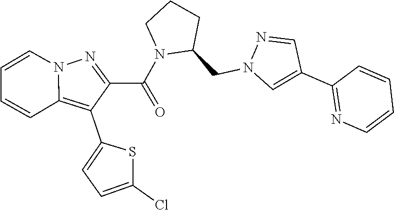 [SMILES: O=C(c1nn2ccccc2c1-c1ccc(Cl)s1)N1CCC[C@H]1Cn1cc(-c2ccccn2)cn1] |
| 254 | 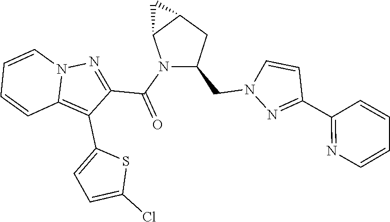 [SMILES: O=C(c1nn2ccccc2c1-c1ccc(Cl)s1)N1[C@H](Cn2ccc(-c3ccccn3)n2)C[C@H]2C[C@H]21] |
| 255 | 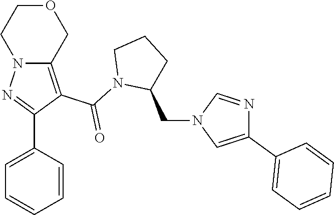 [SMILES: O=C(c1c(-c2ccccc2)nn2c1COCC2)N1CCC[C@H]1Cn1cnc(-c2ccccc2)c1] |
| 256 | 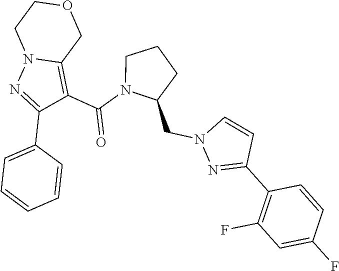 [SMILES: O=C(c1c(-c2ccccc2)nn2c1COCC2)N1CCC[C@H]1Cn1ccc(-c2ccc(F)cc2F)n1] |
| 257 | 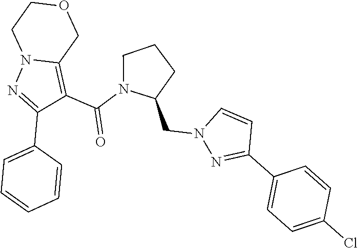 [SMILES: O=C(c1c(-c2ccccc2)nn2c1COCC2)N1CCC[C@H]1Cn1ccc(-c2ccc(Cl)cc2)n1] |
| 258 | 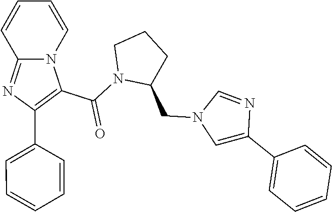 [SMILES: O=C(c1c(-c2ccccc2)nc2ccccn12)N1CCC[C@H]1Cn1cnc(-c2ccccc2)c1] |
| 259 | 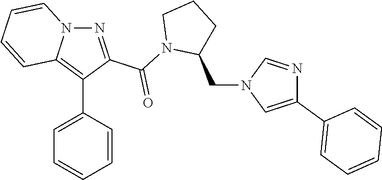 [SMILES: O=C(c1nn2ccccc2c1-c1ccccc1)N1CCC[C@H]1Cn1cnc(-c2ccccc2)c1] |
| 260 | 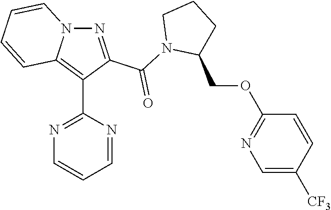 [SMILES: O=C(c1nn2ccccc2c1-c1ncccn1)N1CCC[C@H]1COc1ccc(C(F)(F)F)cn1] |
| 261 | 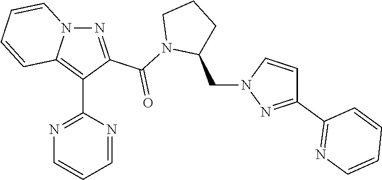 [SMILES: O=C(c1nn2ccccc2c1-c1ncccn1)N1CCC[C@H]1Cn1ccc(-c2ccccn2)n1] |
| 262 | 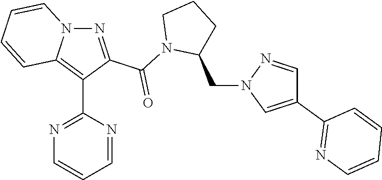 [SMILES: O=C(c1nn2ccccc2c1-c1ncccn1)N1CCC[C@H]1Cn1cc(-c2ccccn2)cn1] |
| 263 | 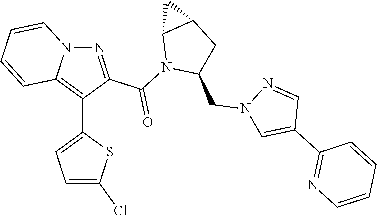 [SMILES: O=C(c1nn2ccccc2c1-c1ccc(Cl)s1)N1[C@H](Cn2cc(-c3ccccn3)cn2)C[C@H]2C[C@H]21] |

More preferred compounds of this disclosure are those presented below as Examples 4, 6, 7, 8, 10, 12, 13, 20, 22, 24, 25-29, 34, 40, 42-50, 53-64, 66-69, 73, 75, 78, 80, 89, 90, 92, 94, 95, 97, 107, 111, 112, 117-119, 122-142, 147-151, 156, 158, 171-183, 185-198, 201-203, 205, 207, 208, 210-214, 223, and 224.

The most preferred compounds of this disclosure are those presented below as Examples 53, 55, 66, 95, 112, 118, 119, 122, 123, 124, 129, 130, 131, 134, 135, 138, 139, 140, 141, 142, 147, 148, 156, 171, 172, 173, 174, 175, 176, 177, 178, 179, 180, 181, 182, 191, 195, 203, 205, 211, 223, and 224.

Any embodiment given herein is also intended to represent unlabeled forms as well as isotopically labeled forms of the compounds, unless otherwise indicated. Isotopically labeled compounds have structures depicted by the Formulas given herein except that one or more atoms are replaced by an atom having a selected atomic mass or mass number. Examples of isotopes that can be incorporated into compounds of the disclosure include isotopes of hydrogen, carbon, nitrogen, oxygen, phosphorous, fluorine, and chlorine, such as ²H, ³H, ¹¹C, ¹³C, ¹⁴C, ¹⁵N, ¹⁸F, ³¹P, ³²P, ³⁵S, ³⁶Cl, ¹²⁵I, respectively. The disclosure includes various isotopically labeled compounds as defined herein, for example those into which radioactive isotopes, such as ³H, ¹³C, and ¹⁴C, are present. Such isotopically labelled compounds are useful in metabolic studies (preferably with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. In particular, an ¹⁸F or labeled compound may be particularly preferred for PET or SPECT studies. Isotopically labeled compounds of this disclosure and prodrugs thereof can generally be prepared by carrying out the procedures disclosed in the schemes or in the examples and preparations described below by substituting a readily available isotopically labeled reagent for a non-isotopically labeled reagent.

Many of the compounds useful in the methods and compositions of this disclosure have at least one stereogenic center in their structure. This stereogenic center may be present in a R or a S configuration, said R and S notation is used in correspondence with the rules described in Pure Appl. Chem. (1976), 45, 11-30. The disclosure also relates to all stereoisomeric forms such as enantiomeric and diastereoisomeric forms of the compounds or mixtures thereof (including all possible mixtures of stereoisomers). See, e.g., WO 01/062726. Furthermore, multiple substituents on a piperidinyl or pyrrolidinyl ring can also be in either cis or trans relationship to each other with respect to the plane of the piperidinyl or the pyrrolidinyl ring. Such forms or geometric isomers, although not explicitly indicated in the Formulae described herein, are intended to be included within the scope of the present disclosure. With respect to the methods and compositions of the present disclosure, reference to a compound or compounds is intended to encompass that compound in each of its possible isomeric forms and mixtures thereof unless the particular isomeric form is referred to specifically.

Pharmaceutically acceptable salts is used herein to refer to an agent or a compound according to the disclosure that is a therapeutically active, non-toxic base and acid salt form of the compounds. The acid addition salt form of a compound that occurs in its free form as a base can be obtained by treating said free base form with an appropriate acid such as an inorganic acid, for example, a hydrohalic such as hydrochloric or hydrobromic, sulfuric, nitric, phosphoric and the like; or an organic acid, such as, for example, acetic, hydroxyacetic, propanoic, lactic, pyruvic, malonic, succinic, maleic, fumaric, malic, tartaric, citric, methanesulfonic, ethanesulfonic, benzenesulfonic, p-toluenesulfonic, cyclic, salicylic, p-aminosalicylic, pamoic and the like (see, e.g., WO 01/062726, U.S. Pat. No. 8,492,416 B2, US 2017/0022208 A1, and US 2017/0253603 A2).

Compounds containing acidic protons may be converted into their therapeutically active, non-toxic base addition salt form, e. g. metal or amine salts, by treatment with appropriate organic and inorganic bases. Appropriate base salt forms include, for example, ammonium salts, alkali and alkaline earth metal salts, e. g., lithium, sodium, potassium, magnesium, calcium salts and the like, salts with organic bases, and salts with amino acids such as, for example, arginine, lysine and the like. Conversely, said salt forms can be converted into the free forms by treatment with an appropriate base or acid. Compounds and their salts can be in the form of a solvate, which is included within the scope of the present disclosure. Such solvates include for example hydrates, alcoholates and the like.

Throughout this specification, the word “comprise” or variations such as “comprises” or “comprising” will be understood to imply the inclusion of a stated integer (or components) or group of integers (or components), but not the exclusion of any other integer (or components) or group of integers (or components). The singular forms “a,” “an,” and “the” include the plurals unless the context clearly dictates otherwise. The term “including” is used to mean “including but not limited to “Including” and “including but not limited to” are used interchangeably. The term “Substance Addiction” or “Substance Addiction Disorder” (SUD) can include addiction to a number of either stimulants (for example: including but not limited to cocaine, methamphetamines, nicotine, etc.) or depressants (for example: including but not limited to opioids, alcohol, etc.). The term “agent” is used herein to denote a chemical compound (such as an organic or a mixture of chemical compounds). Agents include, for example, agents that are known with respect to structure, and their orexin and/or kappa-opioid antagonist/modulators activities of such agents may render them suitable as “therapeutic agents” in the methods and compositions of this disclosure. Where the term “=” is used to describe a substituent (e.g., R1), it is understood meant to indicate “is selected from the group consisting of”. Where the term “=” used with reference to multiple possible substituents, it should be understood to mean that each of such possible substituents can be independently selected from the groups listed thereafter (e.g., “Z1, Z2=” is meant to indicate “Z₁ and Z₂ are independently selected from the group consisting of” the groups listed).

The term “aryl” as used herein means a monocyclic or bicyclic carbocyclic aromatic or aryl ring system. Phenyl is a non-limiting (unless otherwise indicated) example of a monocyclic aromatic or aryl ring system.

The term “heteroaryl” as used herein means a monocyclic or bicyclic aromatic or aryl ring system having 1 to 3 heteroatom or heteroatom groups in each ring selected from O, N, NH or S in a chemically stable arrangement. In such a bicyclic aromatic or aryl ring system embodiment of “heteroaryl”: both rings may be aromatic or aryl; and one or both rings may contain said heteroatom or heteroatom groups. Examples of heteroaryl rings include 2-furanyl, 3-furanyl, N-imidazolyl, 2-imidazolyl, 4-imidazolyl, 5-imidazolyl, benzimidazolyl, 3-isoxazolyl, 4-isoxazolyl, 5-isoxazolyl, 2-oxazolyl, 4-oxazolyl, 5-oxazolyl, N-pyrrolyl, 2-pyrrolyl, 3-pyrrolyl, 2-pyridyl, 3-pyridyl, 4-pyridyl, 2-pyrimidinyl, 4-pyrimidinyl, 5-pyrimidinyl, pyridazinyl (e.g., 3-pyridazinyl), 2-thiazolyl, 4-thiazolyl, 5-thiazolyl, tetrazolyl (e.g., 5-tetrazolyl), triazolyl (e.g., 2-triazolyl and 5-triazolyl), 2-thienyl, 3-thienyl, benzofuryl, benzothiophenyl, indolyl (e.g., 2-indolyl), pyrazolyl (e.g., 2-pyrazolyl), isothiazolyl, 1,2,3-oxadiazolyl, 1,2,5-oxadiazolyl, 1,2,4-oxadiazolyl, 1,2,3-triazolyl, 1,2,3-thiadiazolyl, 1,3,4-thiadiazolyl, 1,2,5-thiadiazolyl, purinyl, pyrazinyl, 1,3,5-triazinyl, quinolinyl (e.g., 2-quinolinyl, 3-quinolinyl, 4-quinolinyl), and isoquinolinyl (e.g., 1-isoquinolinyl, 3-isoquinolinyl, or 4-isoquinolinyl).

The term “cycloalkyl or cycloalkenyl” refers to a monocyclic or fused or (C1-3)alkyl bridged bicyclic carbocyclic ring system that is not aromatic or aryl. Cycloalkenyl rings have one or more units of unsaturation. Preferred cycloalkyl or cycloalkenyl groups include cyclopropyl, cyclobutyl, cyclopentyl, cyclohexyl, cyclohexenyl, cycloheptyl, cycloheptenyl, norbornyl, adamantly and decalinyl.

The compounds of the present disclosure also include prodrugs, analogs or derivatives. The term “prodrug” is a recognized art in the field and is intended to encompass compounds or agents which, under physiological conditions, are converted into orexin and kappa-opioid antagonists. A common method for making a prodrug is to select moieties which are hydrolyzed or metabolized under physiological conditions to provide the desired compound or agent. In other embodiments, the prodrug is converted by an enzymatic activity of the host animal to an orexin and/or kappa-opioid antagonist (i.e., as an antagonist and/or modulator thereof).

The present disclosure also includes isotopically labelled, especially ²H (deuterium) labelled compounds of all Formulas, which compounds are identical to the compounds of any of the Formulas described herein except that one or more atoms have each been replaced by an atom having the same atomic number but an atomic mass different from the atomic mass usually found in nature. Isotopically labelled, especially ²H (deuterium) labelled compounds of all Formulas and salts thereof are within the scope of the present disclosure. Substitution of hydrogen with the heavier isotope ²H (deuterium) may lead to greater metabolic stability resulting e.g. in increased in-vivo half-life or reduced dosage requirements, or may lead to reduced inhibition of cytochrome P450 enzymes, resulting e.g. in an improved safety profile. In another aspects of embodiment of the disclosure, the compounds of all Formulas are not isotopically labelled. However, isotopically labelled compounds of all Formula could be prepared by anyone skilled in the art in analogy to the methods described hereinafter, but using the appropriate isotopic variation of suitable reagents or starting materials.

In some embodiments, this disclosure provides a composition comprising any one or more of such a compound, and/or pharmaceutically acceptable salt, hydrate, solvate, polymorph, isomer, or combination thereof. The disclosure further provides pharmaceutical compositions comprising one or more compounds of the disclosure together with a pharmaceutically acceptable carrier or excipient. In some embodiments, this disclosure provides a pharmaceutical composition comprising a compound, pharmaceutically acceptable salt, hydrate, solvate, polymorph, isomer, or combination thereof; and at least one pharmaceutically acceptable excipient, carrier, adjuvant, or vehicle. In some embodiments, this disclosure provides a therapeutically effective amount of such a compound, or a pharmaceutically acceptable salt, hydrate, solvate, polymorph, isomer, or combination thereof. In some embodiments, this disclosure provides such a pharmaceutical composition further comprises at least one second therapeutic agent. The disclosure also provides pharmaceutical compositions comprising one or more compounds of this disclosure (or the like, such as a pharmaceutically acceptable salt thereof) (i.e., as an active agent, as a therapeutic agent), and one or more pharmaceutically acceptable carriers or excipients. A pharmaceutical composition contains a therapeutically effective amount of one or more of such compounds or the like (i.e., active agent(s)), or an appropriate fraction thereof. A composition can optionally contain an additional active agent. In some embodiments, a peptide product is at least about 90%, 95% or 98% pure. Pharmaceutically acceptable excipients and carriers include pharmaceutically acceptable substances, materials and vehicles. Non-limiting examples of types of excipients include liquid and solid fillers, diluents, binders, lubricants, glidants, surfactants, dispersing agents, disintegration agents, emulsifying agents, wetting agents, suspending agents, thickeners, solvents, isotonic agents, buffers, pH adjusters, absorption-delaying agents, stabilizers, antioxidants, preservatives, antimicrobial agents, antibacterial agents, antifungal agents, chelating agents, adjuvants, sweetening agents, flavoring agents, coloring agents, encapsulating materials and coating materials. The use of such excipients in pharmaceutical Formulations is known in the art. For example, conventional vehicles and carriers include without limitation oils (e.g., vegetable oils such as olive oil and sesame oil), aqueous solvents (e.g., saline, buffered saline (e.g., phosphate-buffered saline [PBS]) and isotonic solutions (e.g., Ringer's solution)}, and organic solvents (e.g., dimethyl sulfoxide and alcohols (e.g., ethanol, glycerol and propylene glycol)). Except insofar as any conventional excipient or carrier is incompatible with a peptide product, the disclosure encompasses the use of conventional excipients and carriers in Formulations containing a peptide product. See, e.g., Remington: The Science and Practice of Pharmacy, 2lst Ed., Lippincott Williams and Wilkins (Philadelphia, Pennsylvania) (2005); Handbook of Pharmaceutical Excipients, 5th Ed., Rowe et ah, Eds., The Pharmaceutical Press and the American Pharmaceutical Association (2005); Handbook of Pharmaceutical Additives, 3rd Ed., Ash and Ash, Eds., Gower Publishing Co. (2007); and Pharmaceutical Pre-Formulation and Formulation, Gibson, Ed., CRC Press (Boca Raton, Florida) (2004). The appropriateness of a particular Formulation can depend on various factors, such as the route of administration chosen. Potential routes of administration of a pharmaceutical composition comprising the compounds or the like disclosed herein can include, without limitation oral, parenteral (including intradermal, subcutaneous, intramuscular, intravascular, intravenous, intra-arterial, intraperitoneal, intracavitary and topical), topical (including transdermal, transmucosal, intranasal (e.g., by nasal spray or drop), ocular (e.g., by eye drop), pulmonary (e.g., by oral or nasal inhalation), buccal, sublingual, rectal (e.g., by suppository), vaginal (e.g., by suppository), and/or other suitable route as would be known to those of ordinary skill in the art.

In some embodiments, the compounds and compositions of this disclosure can be used as orexin receptor and/or K-opioid receptor (or kappa opioid receptor, abbreviated KOR or KOP for its ligand ketazocine, which is a G protein-coupled receptor that in humans is encoded by the OPRK1 gene) antagonists and/or modulators. In preferred embodiments, the compounds and/or compositions disclosed herein can be antagonists for one or more orexin receptor antagonists, such as either one of OX₁R or OX₂R, or both OX₁R or OX₂R; and antagonists and/or modulators of KOR. The compounds and/or compositions disclosed herein can be selectively more antagonistic for one or more orexin receptor as compared to another orexin receptor, such as in being more or less antagonistic of OX₁R or OX₂R as compared to the other orexin receptor. Thus, the compounds and/or compositions of the same can be referred to herein as “Orexin Receptor Antagonists”. In some embodiments, the compounds may be antagonists and/or modulators of the K-opioid receptor. In some embodiments, the compounds and/or compositions of this disclosure may be antagonists of one or more types orexin receptors (i.e., Orexin Receptor Antagonist) but not a KOR. In some embodiments, for instance, one or more compounds of this disclosure, and/or a combination thereof, designed and preferred to provide the following characteristics as measured using standard in vitro cellular assays (e.g., those described in the Examples herein), for effective therapeutic efficacy: 1) OX₁R Kb<100 nM, OX₂R Kb>500 nM, KOR Ki<1000 nM; 2) OX₁R Kb<100 nM, OX₂R Kb<1000 nM, KOR Ki<1000 nM; or, 3) OX₁R Kb<100 nM, OX₂R Kb>10000 nM, KOR Ki<1000 4) OX₁R Kb<100 nM, OX₂R Kb<1000 nM, KOR Ki<10000 nM. In some embodiments, this disclosure provides methods of preventing or treating a condition associated with an orexin receptor (i.e., as an Orexin Receptor Antagonist) and/or one or more K-opioid receptors. For in vivo assays, the compounds and/or compositions of this disclosure may be tested using an animal model such as the rat by measuring primary dependent measures (e.g., such as but not limited to the time to obtain first drug injection, total drug injections taken, rate of drug intake, and total inactive lever presses), the progressive ratio (e.g, such as but not limited to time to obtain first drug injection, breakpoints, final ratio completed, and total active and inactive lever presses), and reinstatement (e.g., such as but not limited to time to first lever press as well as total active and inactive lever presses). These tests and in-vivo assessments are designed, planned and expected to demonstrate disclosures of compounds therapeutic utility and in vivo efficacy—towards attenuation of intake and motivation towards illicit drugs of abuse using techniques known to those of ordinary skill in the art (e.g., Brain Research 1731 (2020), edited by James, et al. (see, e.g., Brodnik, et al. Article 145894); Gentile, et al. Addict Biol 2018, 23(1):247-255; Brodnik, et al. Behav Brain Res. 2015 September 15; 291: 377-384, doi:10.1016/j.bbr.2015.05.051). Other testing methods may also be suitable as would be understood by those of ordinary skill in the art.

In some embodiments, this disclosure provides methods of preventing or treating a condition selected from the group consisting of a central nervous system (CNS) disorder, substance addiction, dependence, panic, anxiety, depression, posttraumatic stress disorder (PTSD), neurodegeneration, autism, schizophrenia, and Alzheimer disease (AD) in a subject in need thereof, by administering to the subject any of such one or more compounds and/or composition comprising one or more of such compounds, or pharmaceutically acceptable salt, hydrate, solvate, polymorph, isomer, or combination thereof. In some embodiments, the methods can include administering a composition comprising a therapeutically effective amount of the compound, pharmaceutically acceptable salt, hydrate, solvate, polymorph, isomer, or combination thereof. In some embodiments, the composition comprises a pharmaceutically acceptable salt or isotope of such a compound. In some embodiments, the composition can comprise an unlabeled form of the compound or an isotopically labeled form of the compound in which the compound has a structure depicted by the Formula wherein one or more atoms are replaced by an atom having a selected atomic mass or mass number. In some embodiments, this disclosure provides for the use of a compound, pharmaceutically acceptable salt, hydrate, solvate, polymorph, isomer, or combination thereof, disclosed herein in the preparation of a medicament for preventing and/or treating a condition selected from the group consisting of a central nervous system (CNS) disorder, substance addiction, dependence, panic, anxiety, depression, posttraumatic stress disorder (PTSD), neurodegeneration, autism, schizophrenia, and Alzheimer disease (AD) in a subject in need thereof. Substance addiction, for instance, can include addiction of a person to one or more of an opioids (e.g., such as but not limited to heroin, morphine, oxycodone (e.g., OxyContin, Percocet), fentanyl, and/or hydrocodone (e.g., Vicodin)), one or more stimulants (e.g., amphetamine (e.g., Adderall, Ritalin), cocaine, crack cocaine, methamphetamine, one or more sedatives and/or tranquilizers (e.g., such as but not limited to benzodiazepines (e.g., Valium, Xanax, Klonopin) or barbituates (e.g., Nembutal, Luminal, phenobarbital), or other addictive substance(s) as may be known to those of ordinary skill in the art. In some embodiments, the use can include a composition comprises a therapeutically effective amount of the compound, pharmaceutically acceptable salt, hydrate, solvate, polymorph, isomer, or combination thereof. In some embodiments, such use can comprise a composition a pharmaceutically acceptable salt or isotope of the compound. In some embodiments, such use can comprise a composition comprises an unlabeled form of the compound or an isotopically labeled form of the compound in which the compound has a structure depicted by the Formula wherein one or more atoms are replaced by an atom having a selected atomic mass or mass number. This disclosure also provides intermediates of the compounds disclosed herein as well as methods for preparing the same. In some embodiments, such methods for preparing can include using any of the intermediates disclosed herein. Other embodiments are also contemplated here as would be understood by those of ordinary skill in the art.

The term “therapeutically effective amount” refers to an amount of a compound that, when administered to a subject, is sufficient to prevent, reduce the risk of developing, delay the onset of, slow the progression of or cause regression of the medical condition being treated, or to alleviate to some extent the medical condition or one or more symptoms or complications of that condition, at least in some fraction of the subjects taking that compound. The term “therapeutically effective amount” also refers to an amount of a compound that is sufficient to elicit the biological or medical response of a cell, tissue, organ or human which is sought by a medical doctor or clinician. The terms “treat,” “treating” and “treatment” include alleviating, ameliorating, inhibiting the progress of, reversing or abrogating a medical condition or one or more symptoms or complications associated with the condition, and alleviating, ameliorating or eradicating one or more causes of the condition. Reference to “treatment” of a medical condition includes prevention of the condition. The terms “prevent”, “preventing” and “prevention” include precluding, reducing the risk of developing and delaying the onset of a medical condition or one or more symptoms or complications associated with the condition. The term “medical conditions” (or “conditions” for brevity) includes diseases and disorders. The terms “diseases” and “disorders” are used interchangeably herein.

Throughout this specification the word “comprise” or variations such as “comprises” or “comprising” will be understood to imply the inclusion of a stated integer (or components) or group of integers (or components), but not the exclusion of any other integer (or components) or group of integers (or components). The singular forms “a,” “an,” and “the” include the plurals unless the context clearly dictates otherwise. The symbol “=” when used in describing a Formula means “is”. The term “including” is used to mean “including but not limited to “Including” and “including but not limited to” are used interchangeably. The term “agent” is used herein to denote a chemical compound (such as an organic or a mixture of chemical compounds). Agents include, for example, agents that are known with respect to structure, and their orexin antagonist activities of such agents may render them suitable as “therapeutic agents” in the methods and compositions disclosed herein. In addition, those of ordinary skill in the art recognize that it is common to use the following abbreviations:
Me: methyl
Et: ethyl
t-Bu: tert-butyl
Ar: aryl
Ph: phenyl
BINAP: 2,2′-bis(diphenylphosphino)-1,1′-binaphthyl
Bn: benzyl
Ac: acetyl
Boc: tert-butyloxy carbonyl
BSA: bovine serum albumin
CbzCl: benzylchloroformate
CDI: carbonyl diimidazole
DCM: dichloromethane
DCE: dichloroethane
DEAD: diethylazodicarboxylate
DIPEA: N,N-diisopropylethylamine
DMF: N,N-dimethylformamide
DMSO: dimethylsulfoxide
CH₂Cl₂: dichloromethane
EDC: N-(3-Dimethylaminopropyl)-N′-ethylcarbodiimide
Et₃N: triethylamine
EtOAc: ethyl acetate
EtOH: ethanol
HCl: hydrogen chloride
HOAt: 1-hydroxy-7-aza-10-benzotriazole
HOBT: hydroxybenzotriazole hydrate
LCMS: Liquid Chromatography Mass Spectrometry
HPLC: High Performance Liquid Chromatography
Hunig's base: N,N-diisopropylethylamine
MeOH: methanol
MgS0₄: magnesium sulfate
MTBE: methyl tert-butyl ether
NaHCO₃: sodium bicarbonate
Na2CO3: sodium carbonate
K2CO3: potassium carbonate
NaOH: sodium hydroxide
NMM: N-methylmorpholine
Pt0₂: Platinum oxide
Pd: Palladium
Pd/C: Palladium over carbon
PyClu: 1-(chloro-1 pyrrolidinylmethylene)pyrrolidiniumhexafluorophosphate
RT or rt: room temperature
SOCl₂: thionyl chloride
THF: tetrahydrofuran
TFA: trifluoroacetic acid
X-Phos: 2-(dicyclohexyl-phosphino)-2′,4′,6′-triisopropylbiphenyl
HATU: (1-[Bis(dimethylamino)methylene]-1H-1,2,3-triazolo[4,5-b]pyridinium 3-oxid hexafluorophosphate
NMR: Nuclear Magnetic Resonance
ESI: Electrospray Ionization
MS: Mass spectrometry reaction

Thus, in some embodiments, this disclosure provides compounds of the Formulas shown below, compositions comprising the same, methods for making (e.g., as in the Examples section) and using the same (e.g., to treat disease conditions):

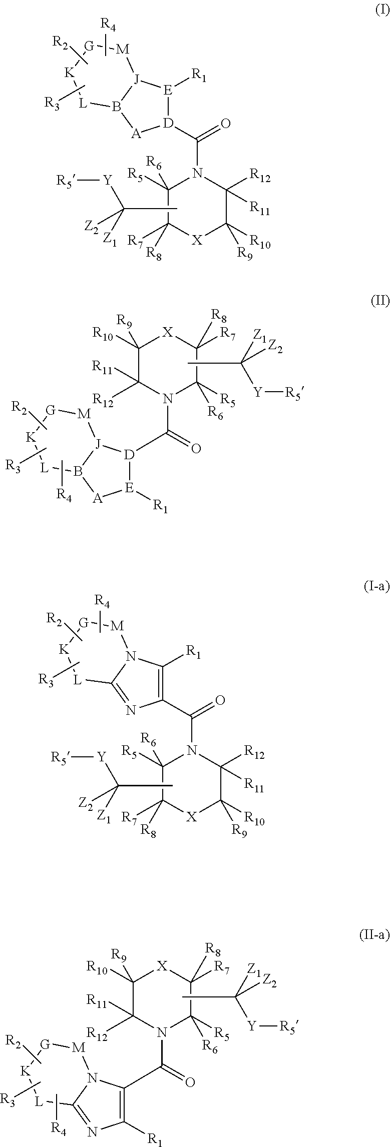

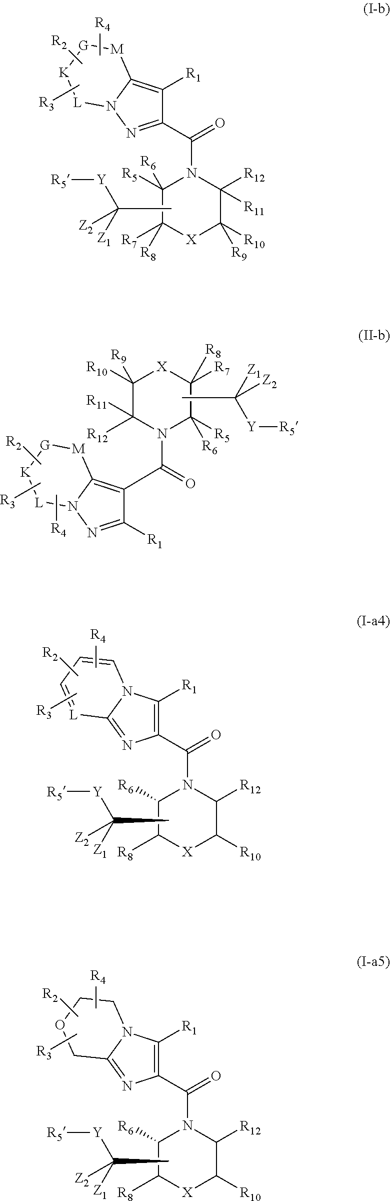

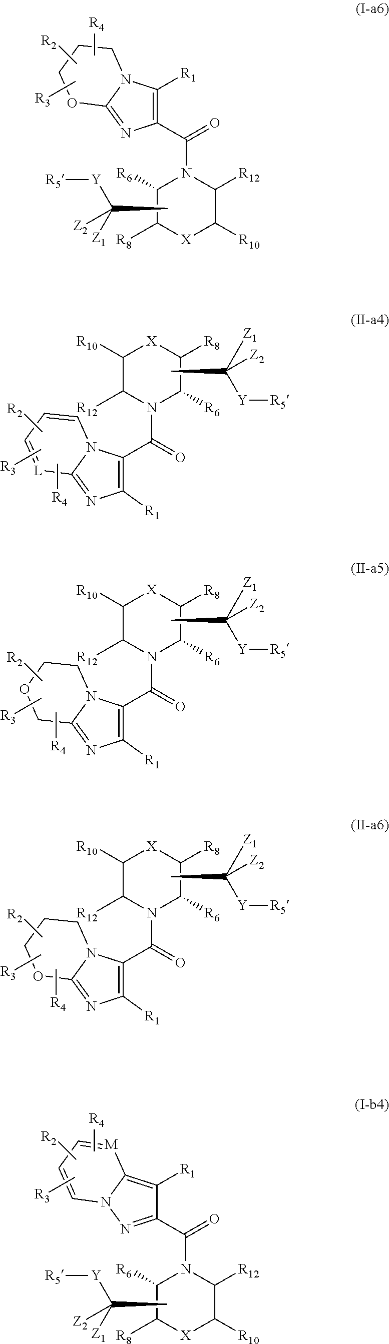

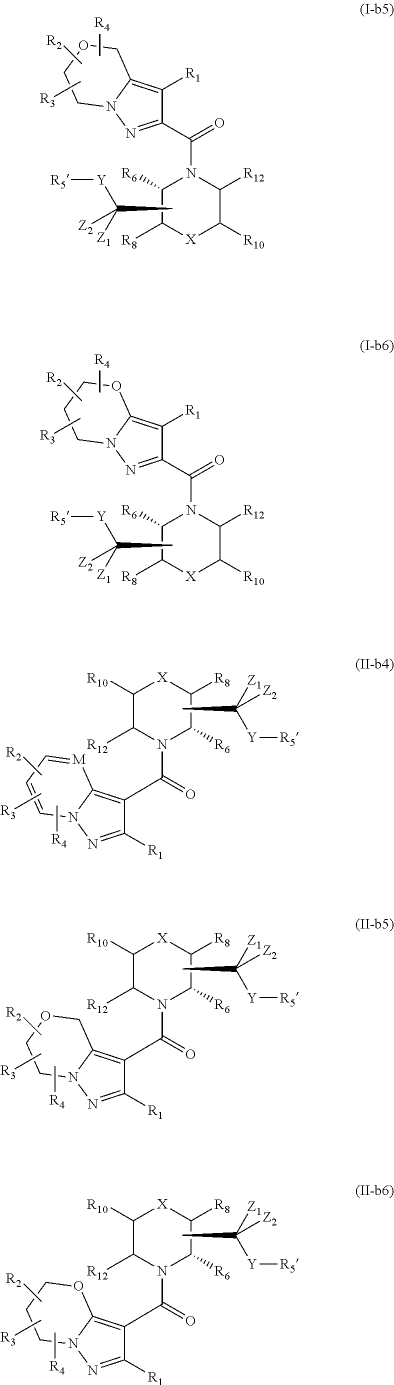

or a pharmaceutically acceptable salt, hydrate, solvate, polymorph, isomer, or combination thereof, wherein, if present:
R₁ includes E as Carbon (C) but not Nitrogen (N), and in Formula I E is linked to J or D with a double bond or in Formula II E is linked to A or D with a double bond, and R₁ is selected from the group consisting of H, alkyl, alkoxy, cycloalkyl, phenyl, aromatic or aryl, heteroaryl that is optionally a 5- or 6 membered heteroaryl, substituted aromatic or aryl, and, substituted heteroaryl that is optionally a 5- or 6-membered heteroaryl; wherein if R₁ is heteroaryl, R₁ is optionally a 5- or 6-membered heteroaryl selected from the group consisting of pyrazolyl, triazolyl, oxazolyl, thiazolyl, thiophenyl, pyridinyl, pyrimidinyl, pyrazinyl, and pyridazinyl; wherein said aromatic, aryl or heteroaryl is unsubstituted, mono-substituted, or di-substituted, wherein the substituents are independently selected from the group consisting of (C₁₋₄)alkyl, (C₁₋₄)alkoxy, halogen, (C₁₋₃)fluoroalkyl, (C₁₋₃)fluoroalkoxy and (C₃₋₇)cycloalkyl;
R₂, R₃, and R₄ are independently selected from the group consisting of H, alkyl, substituted alkyl, (C₁₋₄)alkyl, (C₁₋₄)alkoxy, (C₁₋₃)fluoroalkyl, (C₁₋₃)fluoroalkoxy, (C₃₋₇)cycloalkyl, and halogen that is optionally F, Cl, or Br; wherein each of R₂, R₃ and R₄ is independently and optionally substituted at each substitutable position with up to three (3) substituents independently selected from one, two, or all R₂, R₃ and R₄;
R₅ is selected from the group consisting of H, F, CH₃, alkyl, substituted alkyl, (C₁₋₃)fluoroalkyl, cycloalkyl, and R₅ replaced with the carbon bearing Y, Z₁ and Z₂, wherein R₅′ is as defined herein;
R₅′ is selected from the group consisting of aromatic, aryl, heteroaryl, 5- or 6-membered heteroaryl, substituted aromatic or aryl, substituted heteroaryl or fused two heteroaryl ring systems, optionally comprising a 5- or 6-membered ring; wherein said aromatic, aryl or heteroaryl is unsubstituted, mono-, or di-substituted or tri-substituted, wherein the substituents are independently selected from the group consisting of (C₁₋₄)alkyl, (C₁₋₄)alkoxy, halogen, (C₁₋₃)fluoroalkyl, (C₁₋₃)fluoroalkoxy, (C₃₋₇)cycloalkyl, and (C₃₋₇)heterocycloalkyl;
R₆ is selected from the group consisting of H, F, CH₃, alkyl, substituted alkyl, (C₁₋₃)fluoroalkyl, cycloalkyl, and R₆ connected to either R₁₀ or R₁₁ as alkyl to form a (C₁₋₃)alkyl bridge cyclic structure;
R₇ is selected from the group consisting of H, F, CH₃, alkyl, substituted alkyl, (C₁₋₃)fluoroalkyl, cycloalkyl, and R₇ replaced with the carbon bearing Y, Z₁ and Z₂, wherein R₅′ is defined herein;
R₈ is selected from the group consisting of H, F, CH₃, alkyl, substituted alkyl, (C₁₋₃)fluoroalkyl, cycloalkyl, and R₈ connected to either R₁₀, R₁₁ or R₁₂ as alkyl to form a (C₁₋₃)alkyl bridge cyclic structure;
R₉ is selected from the group consisting of H, F, CH₃, alkyl, substituted alkyl, (C₁₋₃)fluoroalkyl, cycloalkyl, and R₉ connected to either R₆ or R₁₂ as alkyl to form a (C₁₋₃)alkyl bridge cyclic structure;
R₁₀ is selected from the group consisting of H, F, CH₃, alkyl, substituted alkyl, (C₁₋₃)fluoroalkyl, cycloalkyl, and R₁₀ connected to R₆ or R₁₁ as alkyl to form a (C₁₋₃)alkyl bridge cyclic structure;
R₁₁ is selected from the group consisting of H, F, CH₃, alkyl, substituted alkyl, (C₁₋₃)fluoroalkyl, cycloalkyl, and R₁₁ connected to R₆ as alkyl to form a (C₁₋₃)alkyl bridge cyclic structure;
R₁₂ is selected from the group consisting of H, F, CH₃, alkyl, substituted alkyl, (C₁₋₃)fluoroalkyl, cycloalkyl, and R₁₂ connected to R₉ as alkyl to form a (C₁₋₃)alkyl bridge cyclic structure;
X is selected from the group consisting of absent to optionally provide five membered pyrrolidine ring, CH₂, O, and CRₐR_{b} where Rₐ and R_{b} are selected from the group consisting of alkyl, cycloalkyl, and fluoroalkyl; and wherein:
   the carbon atom at position 2 of the piperidine or pyrrolidine is optionally in absolute (S)-configuration; or,
   the carbon atom at position 2 of the of the morpholine ring wherein X is oxygen and optionally in absolute (R)-configuration;
Y is selected from the group consisting of absent to provide R₅′ attached directly to the carbon bearing Z₁ and Z₂ groups; O; NH; CH₂OR₅′; CH₂; NRₐ wherein Rₐ is selected from the group consisting of alkyl, cycloalkyl, and heteroalkyl; and a 5 or 6-membered heteroaryl selected from the group consisting of pyrrolyl, pyrazolyl, triazolyl, oxazolyl, thiazolyl, and oxadiazolyl; and,
Z₁ and Z₂ are independently selected from the group consisting of H, F, (C₁₋₄)alkyl, (C₁₋₃)fluoroalkyl, (C₁₋₃)fluoroalkoxy, and (C₂₋₇)cycloalkyl;
   and wherein:
fused or unfused ring system A-B-J-D-E is a five-membered heteroaryl optionally imidazole wherein A and J are Nitrogen while B, E, D are Carbon; pyrazole wherein A and B are Nitrogen while D, E, and J are Carbon; optionally fused or unfused with one or more additional ring systems;
the fused ring system B-J-M-G-K-L is an arrangement of these outlined variables to provide groups consisting of a 6-membered aromatic, 6-membered aryl, 6-membered substituted aromatic, 6-membered substituted aryl, 6-membered substituted heteroaryl, 6-membered unsubstituted heteroaryl, 5 or 6-membered cycloalkyl, and 5 or 6-membered heterocycloalkyl;
   and wherein, optionally:
A is Nitrogen, optionally an Imidazole or Pyrazole;
B is Carbon or Nitrogen;
J is Carbon or Nitrogen;
D is Carbon;
E is Carbon, wherein R₁ is as defined above;
M is selected from the group consisting of Carbon, CH, CHR₂, CHR₃, CR₂R₃, CR₂, CR₃, CR₄, O, and N;
G is selected from the group consisting of Carbon, CH, CHR₂, CHR₃, CR₂R₃, CR₂, CR₃, CR₄, and O;
K is selected from the group consisting of Carbon, CH, CHR₂, CHR₃, CR₂R₃, CR₂, CR₃, CR₄, and O; and,
L is selected from the group consisting of Carbon, CH, CHR₂, CHR₃, CR₂R₃, CR₂, CR₃, CR₄, O, and N;
or a pharmaceutically acceptable salt, hydrate, solvate, polymorph, isomer, or combination thereof.

In preferred embodiments, the intermediates, reaction conditions and the like used to produce the compounds are described in detail in the Examples section, which is not reproduced in detail here but will be understood by those of skill in the art therefrom as if the same was reproduced in describing this preferred aspect (i.e., incorporated herein). This disclosure also provides methods for manufacturing a pharmaceutical composition comprising combining at least one compound of this disclosure with at least one pharmaceutically acceptable excipient. Methods for making such combinations (i.e., at least one compound or the like and at least one pharmaceutical composition) are generally known in the art and are therefore not described in detail within this aspect, but are incorporated herein.

Thus, this disclosure, in preferred embodiments, provides a compound of any of Examples 1-263 (see Tables 1 and 3), and/or a combination thereof, and/or a pharmaceutically acceptable salt, hydrate, solvate, polymorph, isomer, or combination thereof. In some preferred embodiments, the compound is selected from the group consisting of the compounds of Examples 4, 6, 7, 8, 10, 12, 13, 20, 22, 24, 25-29, 34, 40, 42-50, 53-64, 66-69, 73, 75, 78, 80, 89, 90, 92, 94, 95, 97, 107, 111, 112, 117-119, 122-142, 147-151, 156, 158, 171-183, 185-198, 201-203, 205, 207, 208, 210-214, 223, and 224; and/or a combination thereof, and/or a pharmaceutically acceptable salt, hydrate, solvate, polymorph, isomer, or combination thereof. In the most preferred embodiments, this disclosure provides a compound selected from the group consisting of the compounds of Examples 53, 55, 66, 95, 112, 118, 119, 122, 123, 124, 129, 130, 131, 134, 135, 138, 139, 140, 141, 142, 147, 148, 156, 171, 172, 173, 174, 175, 176, 177, 178, 179, 180, 181, 182, 191, 195, 203, 205, 211, 223, and 224; and/or a combination thereof, and/or pharmaceutically acceptable salt, hydrate, solvate, polymorph, isomer, or combination thereof. In some embodiments, the compound is unlabeled or isotopically. In some embodiments, this disclosure provides a pharmaceutical composition comprising a compound disclosed herein, and/or a combination thereof, and/or a pharmaceutically acceptable salt, hydrate, solvate, polymorph, isomer, or combination thereof; and at least one pharmaceutically acceptable carrier, adjuvant and/or vehicle. In preferred embodiments, the pharmaceutical composition comprises a therapeutically effective amount of the compound, pharmaceutically acceptable salt, hydrate, solvate, polymorph, isomer, and/or combination thereof. In some embodiments, the composition further comprises at least one second therapeutic agent. In some preferred embodiments, this disclosure provides methods for antagonizing and/or modulating at least one orexin receptor and/or at least one kappa-opioid receptor in a cell, comprising the step of exposing the cell to a compound and/or composition of this disclosure, optionally wherein said method is in vitro. In some preferred embodiments, the methods for modulating at least one orexin receptor and/or at least one kappa-opioid receptor in a subject in need thereof, comprising the step of administering to the subject a compound and/or composition of this disclosure. In some preferred embodiments, the method of treating a condition is selected from the group consisting of substance addiction, substance dependence, panic, anxiety, depression, posttraumatic stress disorders (PTSD), neurodegeneration, autism, schizophrenia, pain, Alzheimer diseases (AD), and a central nervous system (CNS) disorder in a subject in need thereof, comprising the step of administering to the subject a compound and/or composition of this disclosure. In some preferred embodiments, the substance corresponding to the substance addiction or substance dependence is selected from the group consisting of one or more opioids, optionally heroin, morphine, oxycodone, fentanyl, and hydrocodone; one or more stimulants optionally selected from the group consisting of amphetamine, cocaine, crack cocaine, and methamphetamine; one or more sedatives and/or tranquilizers, benzodiazepine, and barbituates. In some embodiments, the compound antagonize at least one orexin receptor and/or antagonize or modulate at least one kappa-opioid receptor. In some preferred embodiments, this disclosure provides methods for manufacturing a compound or composition of any preceding claim using at least one applicable combination of acid intermediates, amine intermediates, and methods of presented in Table 2.

All references cited within this disclosure are hereby incorporated by reference in their entirety. Certain embodiments are further described in the following examples. These embodiments are provided as examples only and are not intended to limit the scope of the claims in any way.

### Examples

For this disclosure, the following intermediates were prepared and used for the synthesis of example compounds claimed herein:

### 1. Carboxylic Containing Intermediates Prepared—Carboxylic Acids Group

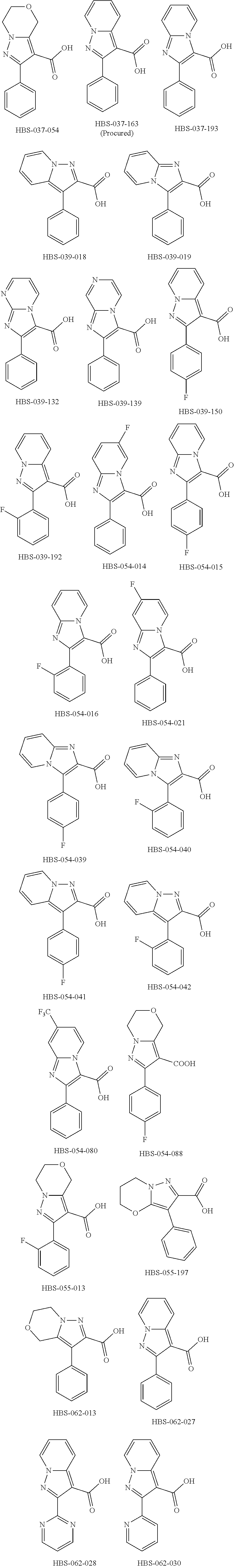

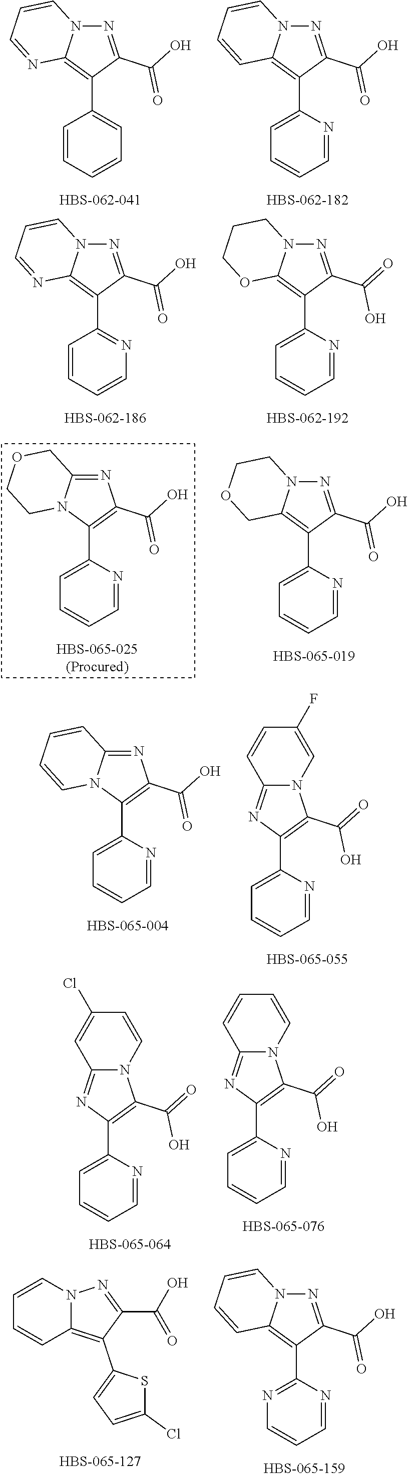

### 2. Secondary Amine Containing Intermediates Prepared—Amine Group

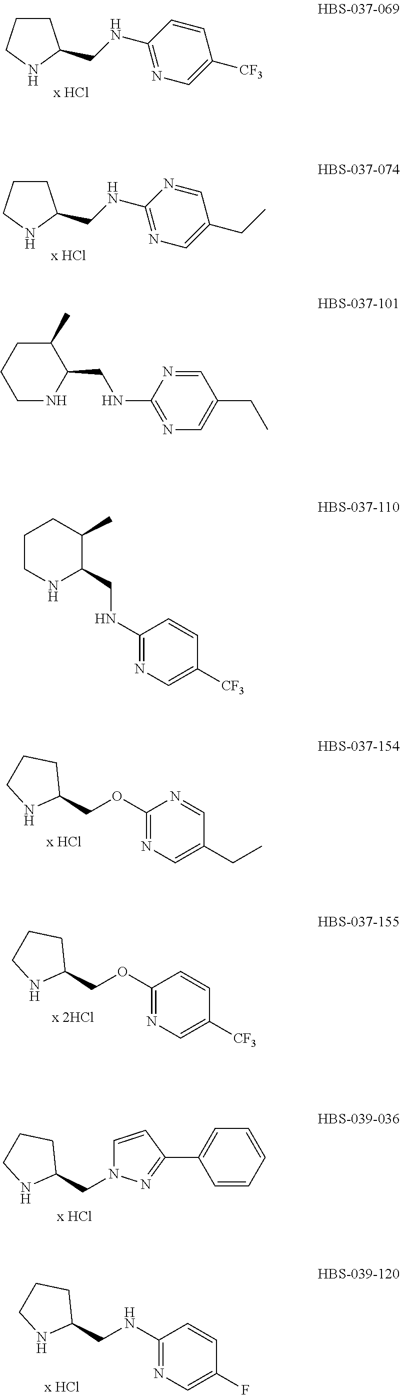

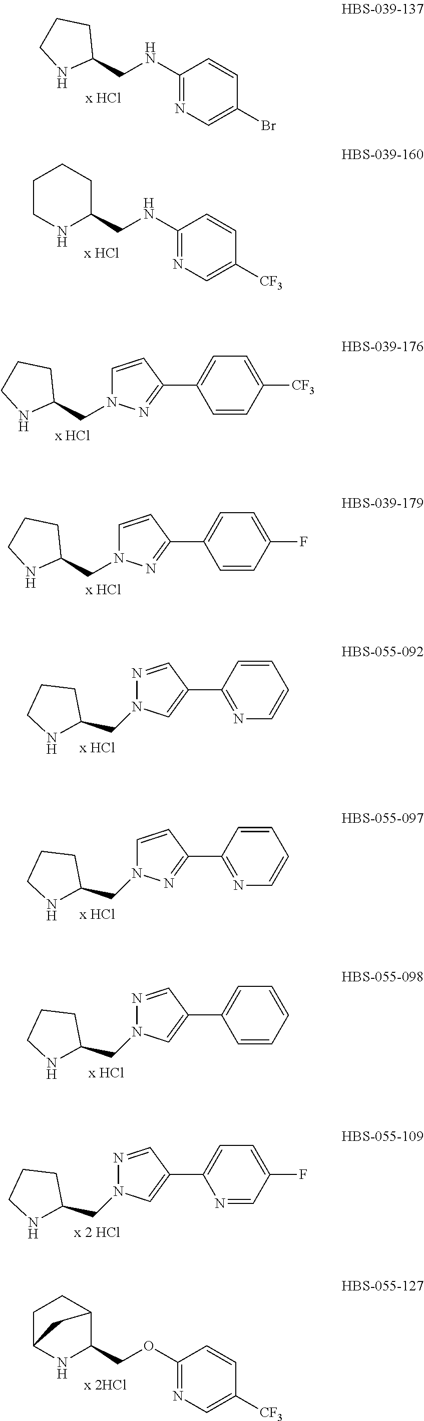

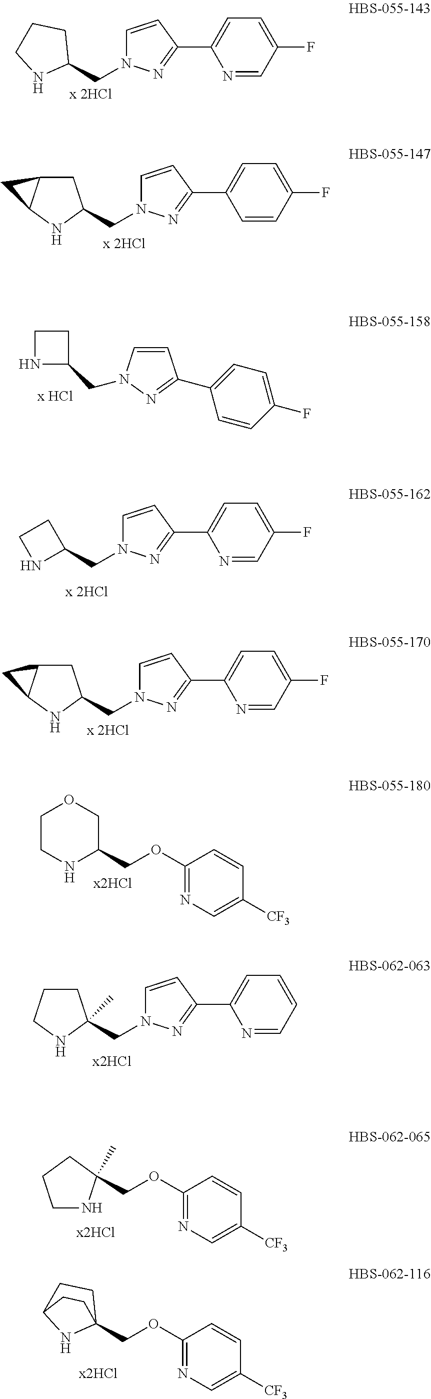

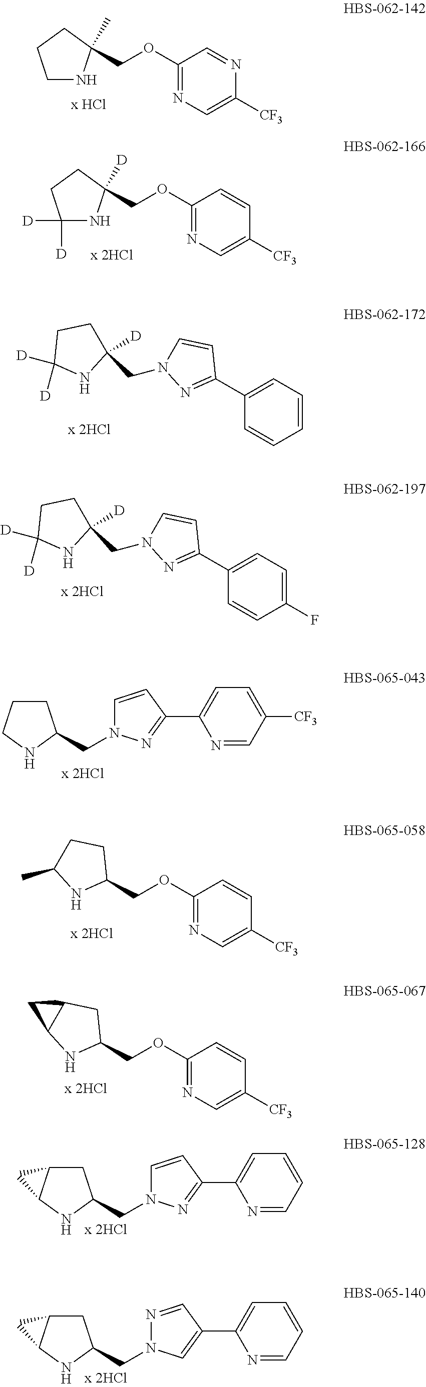

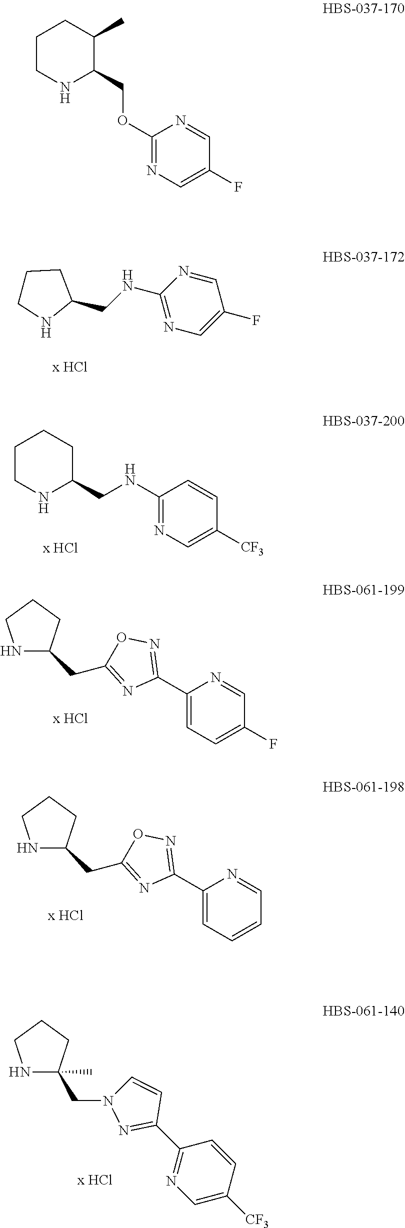 [SMILES: C[C@@H]1CCCN[C@@H]1COc1ncc(F)cn1 ; Fc1cnc(NC[C@@H]2CCCN2)nc1 ; FC(F)(F)c1ccc(NC[C@@H]2CCCCN2)nc1 ; Fc1ccc(-c2noc(C[C@@H]3CCCN3)n2)nc1 ; c1ccc(-c2noc(C[C@@H]3CCCN3)n2)nc1 ; C[C@@]1(Cn2ccc(-c3ccc(C(F)(F)F)cn3)n2)CCCN1]

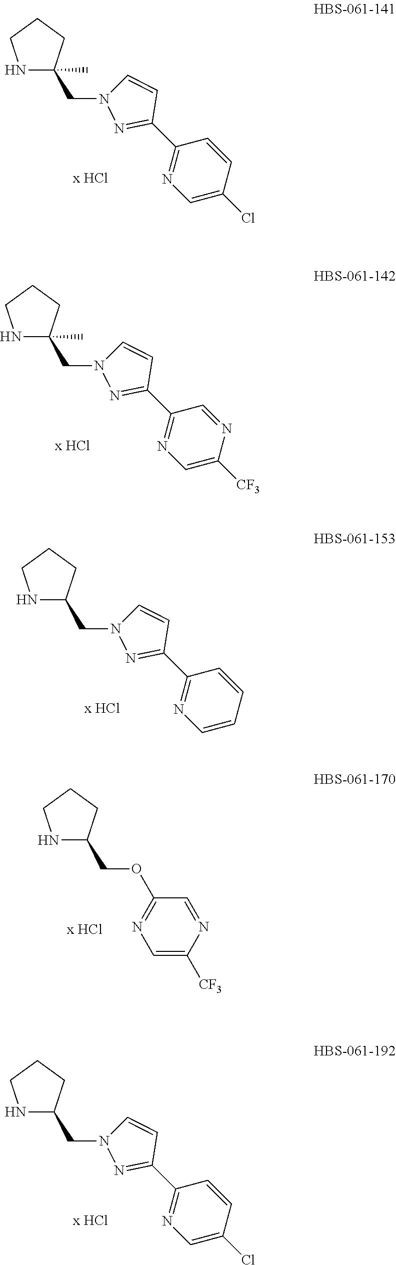 [SMILES: C[C@@]1(Cn2ccc(-c3ccc(Cl)cn3)n2)CCCN1 ; C[C@@]1(Cn2ccc(-c3cnc(C(F)(F)F)cn3)n2)CCCN1 ; c1ccc(-c2ccn(C[C@@H]3CCCN3)n2)nc1 ; FC(F)(F)c1cnc(OC[C@@H]2CCCN2)cn1 ; Clc1ccc(-c2ccn(C[C@@H]3CCCN3)n2)nc1]

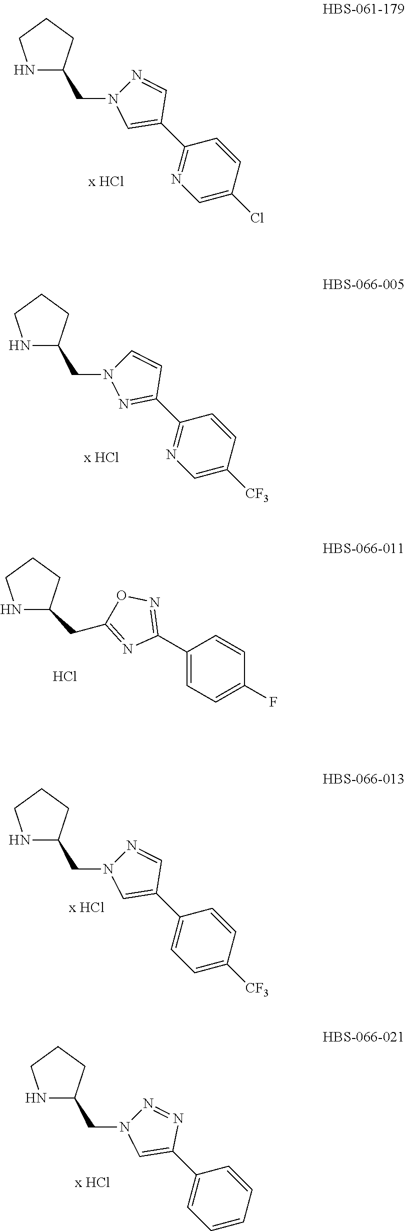 [SMILES: Clc1ccc(-c2cnn(C[C@@H]3CCCN3)c2)nc1 ; FC(F)(F)c1ccc(-c2ccn(C[C@@H]3CCCN3)n2)nc1 ; Fc1ccc(-c2noc(C[C@@H]3CCCN3)n2)cc1 ; Cl ; FC(F)(F)c1ccc(-c2cnn(C[C@@H]3CCCN3)c2)cc1 ; c1ccc(-c2cn(C[C@@H]3CCCN3)nn2)cc1]

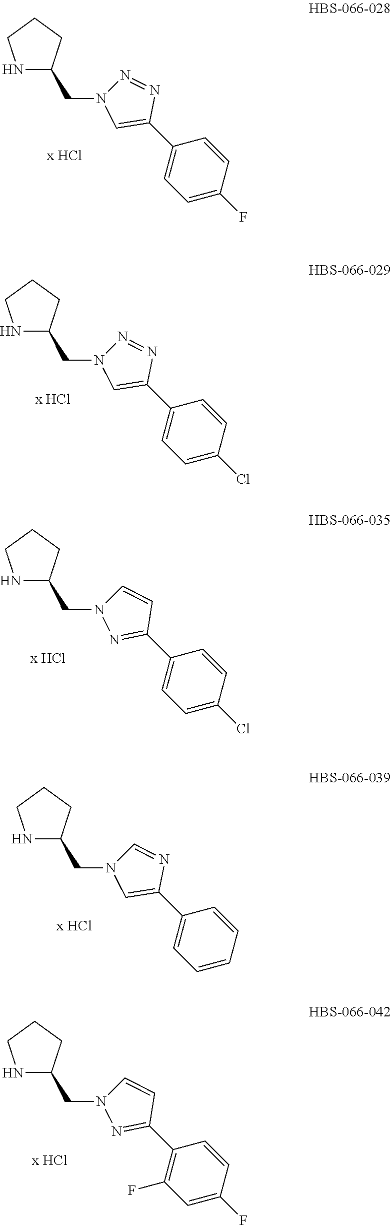 [SMILES: Fc1ccc(-c2cn(C[C@@H]3CCCN3)nn2)cc1 ; Clc1ccc(-c2cn(C[C@@H]3CCCN3)nn2)cc1 ; Clc1ccc(-c2ccn(C[C@@H]3CCCN3)n2)cc1 ; c1ccc(-c2cn(C[C@@H]3CCCN3)cn2)cc1 ; Fc1ccc(-c2ccn(C[C@@H]3CCCN3)n2)c(F)c1]

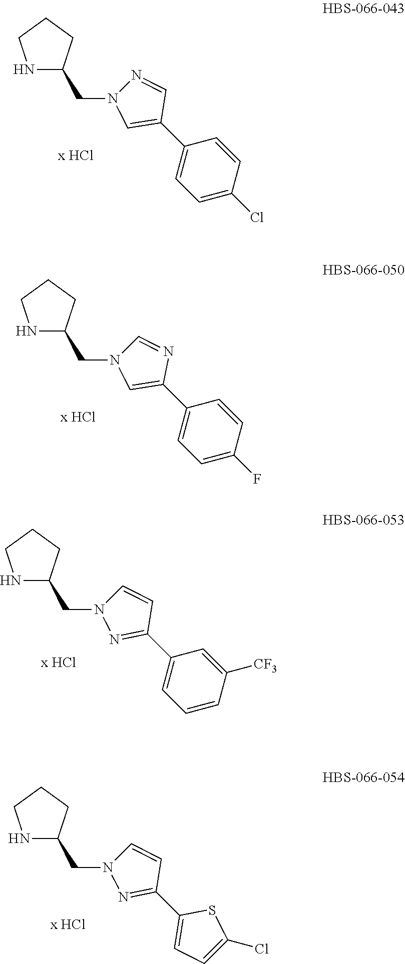 [SMILES: Clc1ccc(-c2cnn(C[C@@H]3CCCN3)c2)cc1 ; Fc1ccc(-c2cn(C[C@@H]3CCCN3)cn2)cc1 ; FC(F)(F)c1cccc(-c2ccn(C[C@@H]3CCCN3)n2)c1 ; Clc1ccc(-c2ccn(C[C@@H]3CCCN3)n2)s1]

### I. General Synthetic Methods and Procedures

### General:

All temperatures are stated in ° C. Commercially available starting materials were used as received without further purification. Unless otherwise specified, all reactions were carried out in oven-dried glassware under an atmosphere of nitrogen. Compounds were purified by flash column chromatography on silica gel or by preparative HPLC. Compounds described in the disclosure are characterized by LC-MS data (retention time t_{R} is given in min; molecular weight obtained from the mass spectrum is given in g/mol) using the conditions listed below.

### LC-MS Under Acidic Conditions

Method A: Agilent 1100 series with mass spectrometry detection (MS: Agilent single quadrupole). Column: Zorbax SB (3.5 μm, 4.6×150 mm). Conditions: MeCN (0.1% FA) [gradient eluent A]; water (0.1% FA) [gradient eluent B]. Gradient: 95% B+5% B over 5 min (flow: 0.8 ml/min). Detection: UV 280/254 nm+MS.

Method B: Agilent 1100 series with mass spectrometry detection (MS: Agilent single quadrupole). Column: X-Bridge C18 (3.5 μm, 4.6×150 mm). Conditions: MeCN (0.1% FA) [gradient eluent A]; water (0.1% FA) [gradient eluent B]. Gradient: 95% B+5% B over 5 min (flow: 0.8 ml/min). Detection: UV 280/254 nm+MS.

In general, the compounds of this disclosure may be prepared by methods known to those skilled in the art and contemporary technologies in the field. Schemes 1-4 below illustrate synthetic routes to the compounds of the present disclosure. Other equivalent schemes, which will be readily apparent to the ordinary skilled synthetic organic or medicinal chemist may alternatively be used to synthesize various portions of the molecules as illustrated by the general schemes described herein.

### Synthesis of Carboxylic Containing Intermediates—Carboxylic Acids Group

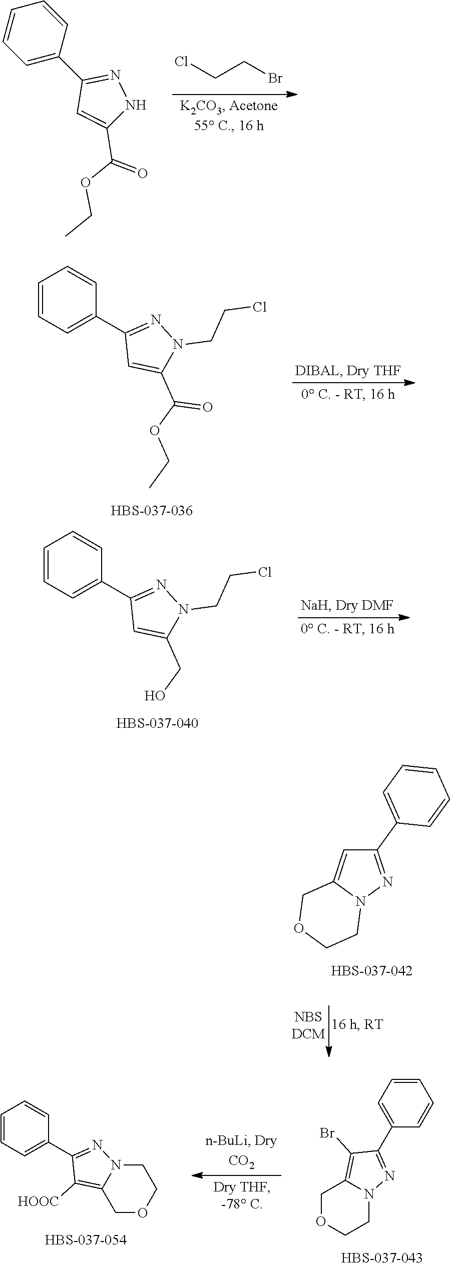

Step 1: Synthesis of HBS-037-036: Ethyl-3-Phenyl-1H-pyrazole-5-carboxylate (0.5 g, 2.31 mmol) was dissolved in Acetone (10.0 mL). The K₂CO₃ (0.96 g, 6.9 mmol) was added, followed by 1-Bromo-2-Chloro-ethane (0.1 mL, 11.6 mmol). The rxn mixture was heated at 55° C. for 16 h. LCMS data shows desired product formation m/z 279.0 and minor amount of side product formation. The rxn mixture was filtered and the solid was washed with ethyl acetate. The filtrate was concentrated under reduced pressure to obtain crude product. The crude product was purified by column chromatography, Mobile Phase: EtOAc:Hexane, gradient. The 0.6 g of liquid product was isolated (Yield 93.2%). MS (ESI) mass calcd. for C₁₄H₁₅ClN₂O₂, 278.7; m/z found 279.0 [M+H]⁺.

Step 2: Synthesis of HBS-037-040: Compound HBS-037-036 (0.55 g, 1.97 mmol) was dissolved in Dry THF (6.0 mL). The DIBAL (12.0 mL, 1.0 M solution, 11.8 mmol) was added, under ice cooling bath. The rxn mixture was gradually warmed to room temperature and stirred for 16 h. LCMS data shows desired product formation m/z 237.0. The rxn mixture was quenched with 1.0 N aq. NaOH solution and diluted with ethyl acetate (10.0 mL). The rxn mixture was filtered through celite bed and washed with ethyl acetate (10.0 mL×3). The EtOAc layer was separated and washed with water followed by brine. The organic layer was dried over anhydrous sodium sulfate. The evaporation of solvent gave crude product 0.4 g (Yield 85.6%). MS (ESI) mass calcd. for C₁₂H₁₃ClN₂O, 236.7; m/z found 237.0 [M+H]⁺.

Step 3: Synthesis of HBS-037-042: Compound HBS-037-040 (0.47 g, 1.97 mmol) was dissolved in Dry DMF (12.0 mL). The NaH (0.12 g, 2.96 mmol) was added under ice cooling. The rxn mixture was gradually warmed to room temperature and stirred for 16 h. LCMS data shows desired product formation m/z 201.1. The rxn mixture was diluted with water and the product was extracted with ethyl acetate. The combined ethyl acetate layer was separated and dried over anhydrous sodium sulfate. The evaporation of solvent gave crude product. The crude product was purified by column chromatography, Mobile Phase: EtOAc:Hexane, gradient. The 0.26 g of solid product was obtained (Yield 65.3%). MS (ESI) mass calcd. for C₁₂H₁₂N₂O, 200.2; m/z found 201.1 [M+H]⁺.

Step 4: Synthesis of HBS-037-043: Compound HBS-037-042 (0.25 g, 1.25 mmol) was dissolved in DCM (5.0 mL). The NBS (0.24 g, 1.37 mmol) was added, and rxn mixture was stirred at room temperature for 16 h. LCMS data shows desired product formation m/z 280.9. The evaporation of solvent gave crude product. The crude product was purified by column chromatography, Mobile Phase: EtOAc:Hexane, gradient. The 0.29 g of liquid product was obtained (Yield 81.7%). MS (ESI) mass calcd. for C₁₂H₁₁BrN₂O, 279.1; m/z found 280.9 [M+H]⁺.

Step 5: Synthesis of HBS-037-054: Compound HBS-037-043 (0.025 g, 0.09 mmol) was dissolved in anhydrous THF (1.0 mL) under N₂ atm. The reaction mixture was cooled at −78.0° C. temperature and n-BuLi (0.12 mL, 1.6 M) was added into the rxn mixture. The rxn mixture was stirred at −78.0° C. temperature for 30.0 min. The dry CO₂ gas bubbled through the rxn mixture at −65° C. and the rxn mixture was gradually warmed at room temperature. The LCMS data shows desired product formation m/z 245, debrominated side product (m/z 201) and some unknown product formation. The rxn mixture was quenched by water and extracted with ethyl acetate. The ethyl acetate layer was separated and debrominated product was recovered. The aq. Layer was acidified with 1 M HCl solution and evaporated to dryness to obtain 0.022 g of solid product. MS (ESI) mass calcd. for C₁₃H₁₂N₂O₃, 244.3; m/z found 245.0 [M+H]⁺, ¹H NMR (400 MHz, CHLOROFORM-d) δ ppm 4.06-4.15 (m, 2H) 4.16-4.25 (m, 2H) 5.03-5.10 (s, 2H) 7.32-7.40 (m, 3H) 7.60-7.69 (m, 2H)

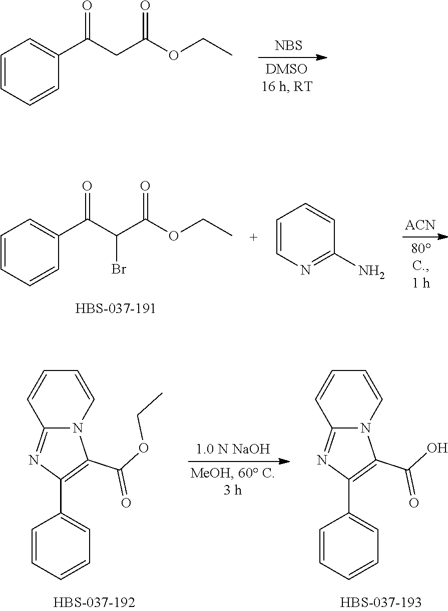

Step 1: Synthesis of HBS-037-191: The ethyl benzoylacetate (0.5 g, 2.6 mmol) was dissolved in DMSO (5.0 mL). The NBS (0.51 g, 2.86 mmol) was added and rxn mixture was stirred at ambient temperature for 24 h. LCMS shows product formation m/z 270.9. The rxn mixture was diluted with water and the product was extracted with ethyl acetate. The ethyl actetate layer was separated and dried over anhydrous Na₂SO₄. The evaporation of solvent gave crude product. The crude product was purified by combi-flash system, Mobile phase: EtOAc:Hexane gradient. The 0.42 g of product was obtained (Yield 59.7%). MS (ESI) mass calcd. for C₁₁H₁₁BrO₃, 271.1; m/z found 270.9 [M+H]⁺.

Step 2: Synthesis of HBS-037-192: Compound HBS-037-191 (0.42 g, 1.55 mmol) was dissolved in anhydrous acetonitrile (5.0 mL). The 2-amino-pyridine (0.15 g, 1.55 mmol) was added and rxn mixture was stirred at 80° C. for 1 h. LCMS shows product formation m/z 267.1. The rxn mixture was concentrated under reduced pressure to yield crude product. The crude product was purified by combi-flash system, Mobile phase: EtOAc:Hexane gradient. The 0.24 g of product was obtained (Yield 58.0%). MS (ESI) mass calcd. for C₁₆H₁₄N₂O₂, 266.3; m/z found 267.1 [M+H]⁺.

Step 3: Synthesis of HBS-037-193: Compound HBS-037-192 (0.24 g, 0.9 mmol) was dissolved in MeOH (5.0 mL). The 1.0 N aq. NaOH solution (4.51 mL, 4.51 mmol) was added and rxn mixture was stirred at 60° C. temperature for 3 h. LCMS shows product formation m/z 239. The rxn mixture was concentrated under reduced pressure. The solid was dissolved in water and acidified with 2.0 M aq. HCl solution (pH=5). The ppts were filtered and washed with water (10.0 mL×3) to obtain 0.2 g of solid product. MS (ESI) mass calcd. for C₁₄H₁₀N₂O₂, 238.2; m/z found 239.1 [M+H]⁺, ¹H NMR (400 MHz, CHLOROFORM-d) δ ppm 7.04 (t, J=6.93 Hz, 1H) 7.33-7.48 (m, 4H) 7.70-7.78 (m, 3H) 9.41 (d, J=7.04 Hz, 1H).

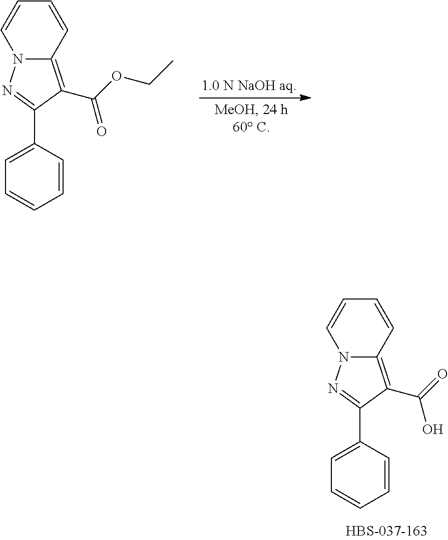 [SMILES: CCOC(=O)c1c(-c2ccccc2)nn2ccccc12 ; O=C(O)c1c(-c2ccccc2)nn2ccccc12]

Step 1: Synthesis of HBS-037-163: 2-Phenyl-pyrazolo[1,5-a]pyridine-3-carboxylic acid ethyl ester (0.2 g, 0.75 mmol) was dissolved in MeOH (6.0 mL). The 1.0 N aq. NaOH solution (3.8 mL, 3.8 mmol) was added and rxn mixture was stirred at 60° C. temperature for 24 h. LCMS shows product formation m/z 239.1. The rxn mixture was concentrated under reduced pressure. The solid was dissolved in water and acidified with 1.0 M aq. HCl solution (pH=5). The ppts were filtered and washed with water (10.0 mL×3) to obtain 0.18 g of solid product. MS (ESI) mass calcd. for C14H10N2O2, 238.2; m/z found 239.1 [M+H]+, 1H NMR (400 MHz, CHLOROFORM-d) δ ppm 6.90-7.03 (m, 1H) 7.27-7.47 (m, 4H) 7.75-7.78 (ddd, J=4.86, 3.21, 1.54 Hz, 2H) 8.18-8.28 (dd, J=8.99, 0.70 Hz, 1H) 8.49-8.56 (dd, J=6.90, 0.73 Hz, 1H).

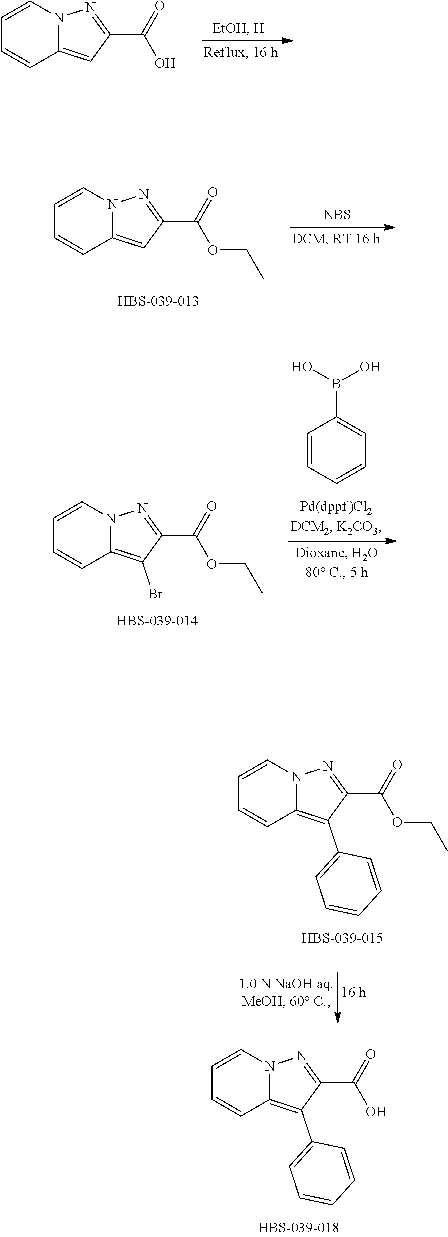

Step 1: Synthesis of HBS-039-013: The pyrazolo[1,5-a]pyridine-2-carboxylic acid (1.0 g, 6.17 mmol) was dissolved in ethanol (20.0 mL). The catalytic amount of conc. sulfuric acid (0.5 mL) was added and rxn mixture was refluxed for 16 h. The LCMS data shows product formation m/z 191.1. The rxn mixture was concentrated under reduced pressure and neutralized with saturate aq. solution of sodium bicarbonate. The product was extracted with ethyl acetate. The ethyl acetate layer was separated and dried over anhydrous Na₂SO₄. The evaporation of solvent gave product. The 1.2 g of product was obtained (Yield Quant.) MS (ESI) mass calcd. for C₁₀H₁₀N₂O₂, 190.2; m/z found 191.1 [M+H]⁺.

Step 2: Synthesis of HBS-039-014: The HBS-039-013 (1.17 g, 6.17 mmol) was dissolved in DCM (25.0 mL). The NBS (1.1 g, 6.17 mmol) was added and rxn mixture was stirred at ambient temperature for 16 h. The LCMS data shows product formation m/z 271.0. The rxn mixture was concentrated under reduced pressure to obtain the crude product. The crude product was purified by ISCO combi-flash chromatography system, Mobile phase: EtOAc:Hexane gradient.

The 1.5 g of product was obtained (Yield 90.4%). MS (ESI) mass calcd. for C₁₀H₉BrN₂O₂, 269.1; m/z found 271.0 [M+H]⁺.

Step 3: Synthesis of HBS-039-015: The HBS-039-014 (0.2 g, 0.74 mmol) was dissolved in mixture of Dioxane/water (8.0:2.0 v/v mL). The phenylboronic acid (0.11 g, 0.89 mmol) and K₂CO₃ (0.3 g, 2.23 mmol) were added followed by Pd(dppf)Cl₂·DCM₂ (0.06 g, 0.07 mmol). The rxn mixture was stirred at 80° C. temperature under N₂ atm. for 5 h. The LCMS data shows product formation m/z 267. The rxn mixture was filtered over celite bed and washed with ethyl acetate. The filtrate was concentrated under reduced pressure to obtain the crude product. The crude product was purified by ISCO combi-flash chromatography system, Mobile phase: EtOAc:Hexane gradient. The 0.2 g of product was obtained (Yield Quant.). MS (ESI) mass calcd. for C₁₆H₁₄N₂O₂, 266.3; m/z found 267.1 [M+H]⁺.

Step 4: Synthesis of HBS-039-018: HBS-039-015 (0.2 g, 0.74 mmol) was dissolved in MeOH (6.0 mL). The 1.0 N aq. solution of NaOH (3.7 mL, 3.7 mmol) was added and rxn mixture was stirred at 60° C. temperature for 16 h. The LCMS data shows product formation m/z 239.1. The rxn mixture was concentrated under reduced pressure and diluted with water. The aq. layer was acidified with 1.0 M aq. HCl solution (pH=5). The ppts were filtered and washed with water (5.0 mL×3) to obtain 0.16 g of solid product (Yield 92.6%). MS (ESI) mass calcd. for C₁₄H₁₀N₂O₂, 238.2; m/z found 239.1 [M+H]⁺.

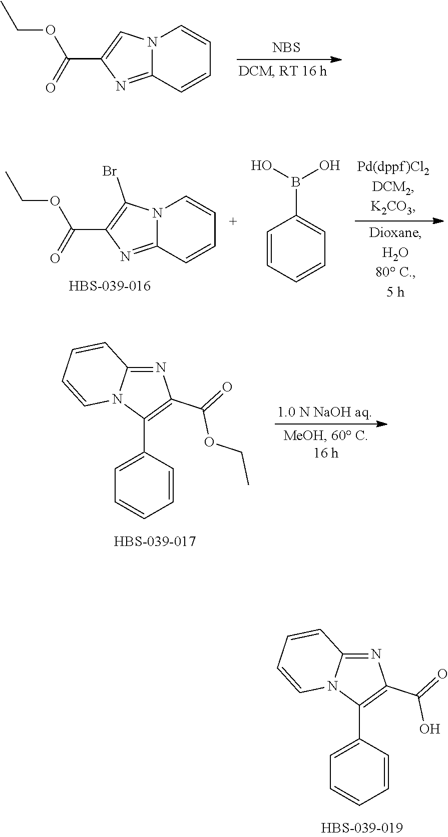

Step 1: Synthesis of HBS-039-016: The Imidazo[1,2-a]pyridine-2-carboxylic acid (1.0 g, 5.26 mmol) was dissolved in DCM (20.0 mL). The NBS (1.0 g, 5.78 mmol) was added and rxn mixture was stirred at ambient temperature for 16 h. The LCMS data shows product formation m/z 271.0. The rxn mixture was concentrated under reduced pressure to obtain the crude product. The crude product was purified by ISCO combi-flash chromatography system, Mobile phase: EtOAc:Hexane gradient. The 1.4 g of product was obtained (Yield 98.9%). MS (ESI) mass calcd. for C₁₀H₉BrN₂O₂, 269.1; m/z found 271.0 [M+H]⁺.

Step 2: Synthesis of HBS-039-017: Compound HBS-039-016 (0.2 g, 0.74 mmol) was dissolved in mixture of Dioxane/water (8.0:2.0 v/v mL). The phenylboronic acid (0.11 g, 0.89 mmol) and K₂CO₃ (0.3 g, 2.23 mmol) were added followed by Pd(dppf)Cl₂·DCM₂ (0.06 g, 0.07 mmol). The rxn mixture was stirred at 80° C. temperature under N₂ atm. for 5 h. The LCMS data shows product formation m/z 267.1. The rxn mixture was filtered over celite bed and washed with ethyl acetate. The filtrate was concentrated under reduced pressure to obtain the crude product. The crude product was purified by ISCO combi-flash chromatography system, Mobile phase: EtOAc:Hexane gradient. The 0.2 g of product was obtained (Yield Quant.). MS (ESI) mass calcd. for C₁₆H₁₄N₂O₂, 266.3; m/z found 267.1 [M+H]⁺.

Step 3: Synthesis of HBS-039-019: Compound HBS-039-017 (0.2 g, 0.74 mmol) was dissolved in MeOH (6.0 mL). The 1.0 N aq. solution of NaOH (3.7 mL, 3.7 mmol) was added and rxn mixture was stirred at 60° C. temperature for 16 h. The LCMS data shows product formation m/z 239.1. The rxn mixture was concentrated under reduced pressure and diluted with water. The aq. layer was acidified with 1.0 M HCl solution (pH=5). The ppts were filtered and washed with water (5.0 mL×3) to obtain 0.11 g of solid product (Yield 62.1%). MS (ESI) mass calcd. for C₁₄H₁₀N₂O₂, 238.2; m/z found 239.1 [M+H]⁺.

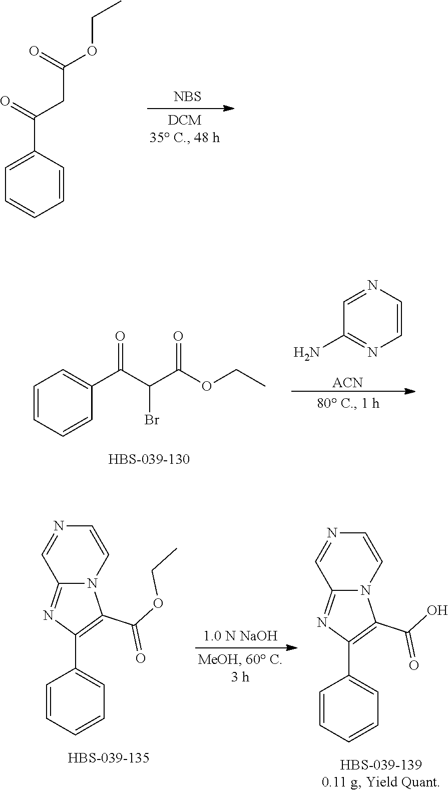

Step 1: Synthesis of HBS-039-130: The ethyl benzoylacetate (3.0 g, 15.61 mmol) was dissolved in DCM (20.0 mL). The NBS (3.1 g, 17.17 mmol) was added and rxn mixture was stirred at 35° C. temperature for 48 h. LCMS shows product formation m/z 272.0. The rxn mixture was diluted with water and the product was extracted with ethyl acetate. The ethyl actetate layer was separated and dried over anhydrous Na₂SO₄. The evaporation of solvent gave crude product. The crude product was purified by combi-flash system, Mobile phase: EtOAc:Hexane gradient. The 3.75 g of product was obtained (Yield 88.6%). MS (ESI) mass calcd. for C₁H₁₁BrO₃, 271.1; m/z found 272.0 [M+H]⁺.

Step 2: Synthesis of HBS-039-135: Compound HBS-039-130 (0.25 g, 0.92 mmol) was dissolved in anhydrous acetonitrile (5.0 mL). The pyrazine-2-amine (0.088 g, 0.92 mmol) was added and rxn mixture was stirred at 80° C. for 24 h. LCMS shows product formation m/z 268.1. The rxn mixture was concentrated under reduced pressure to yield crude product. The crude product was purified by combi-flash system, Mobile phase: EtOAc:Hexane gradient. The 0.13 g of product was obtained (Yield 51.5%). MS (ESI) mass calcd. for C₁₅H₁₃N₃O₂, 267.28; m/z found 268.1 [M+H]⁺.

Step 3: Synthesis of HBS-039-139: Compound HBS-039-135 (0.12 g, 0.45 mmol) was dissolved in MeOH (2.5 mL). The 1.0 N aq. NaOH solution (2.24 mL, 2.24 mmol) was added and rxn mixture was stirred at 60° C. temperature for 3 h. LCMS shows product formation m/z 240.0. The rxn mixture was concentrated under reduced pressure. The solid was dissolved in water and acidified with 2.0 M aq. HCl solution (pH=5). The ppts were filtered and washed with water (1.0 mL×3) to obtain 0.11 g of solid product. MS (ESI) mass calcd. for C₁₃H₉N₃O₂, 239.23; m/z found 240.0 [M+H]⁺.

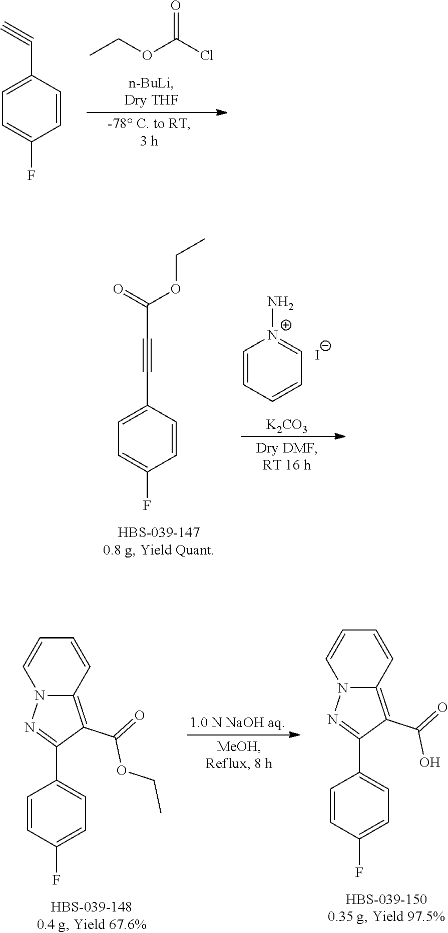

Step 1: Synthesis of HBS-039-147: 1-Ethynyl-4-fluorobenzene (0.5 g, 4.16 mmol) was dissolved in anhydrous THF (5.0 mL). The n-BuLi (5.2 mL, 8.32 mmol) was added at −78° C. temperature and rxn mixture was stirred at −78° C. temperature for 1 h. The ethyl chloroformate (1.59 mL, 16.7 mmol) was added at −78° C. temperature and rxn mixture was gradually warmed to ambient temperature. The LCMS data shows product formation m/z 193.0. The rxn mixture was diluted with aq. NH₄Cl solution and product was extracted with ethyl acetate. The combined ethyl acetate layer was separated and dried over anhydrous sodium sulfate. The evaporation of solvent gave crude product. The 0.8 g of crude product was obtained (Yield Quant.). MS (ESI) mass calcd. for C₁₁H₉FO₂, 192.19; m/z found 193.0 [M+H]⁺.

Step 2: Synthesis of HBS-039-148: Compound HBS-039-147 (0.4 g, 2.08 mmol) and 1-Aminopyridinium iodide (0.46 g, 2.08 mmol) were dissolved in anhydrous DMF (5.0 mL). The anhydrous K₂CO₃ (0.72 g, 5.2 mmol) was added, and reaction mixture was stirred at ambient temperature for 16 h. The LCMS data shows product formation m/z 285.1. The reaction mixture was diluted with water and ppts were filtered. The drying of ppts gave crude product. The crude product was purified by combi-flash system, Mobile phase: EtOAc:Hexane gradient. The 0.4 g of product was obtained (Yield 67.6%). MS (ESI) mass calcd. for C₁₆H₁₃FN₂O₂, 284.29; m/z found 285.1 [M+H]⁺.

Step 3: Synthesis of HBS-039-150: Compound HBS-039-148 (0.4 g, 1.41 mmol) was dissolved in MeOH (7.0 mL). The 1.0 N aq. NaOH solution (7.0 mL, 7.0 mmol) was added, and reaction mixture was refluxed for 8 h. LCMS shows product formation m/z 257.0. The reaction mixture was concentrated under reduced pressure. The solid was dissolved in water and acidified with 2.0 M aq. HCl solution (pH=5). The ppts were filtered and washed with water (5.0 mL×3) to obtain 0.35 g of solid product (Yield 97.5%). MS (ESI) mass calcd. for C₁₄H₉FN₂O₂, 256.23; m/z found 257.0 [M+H]⁺.

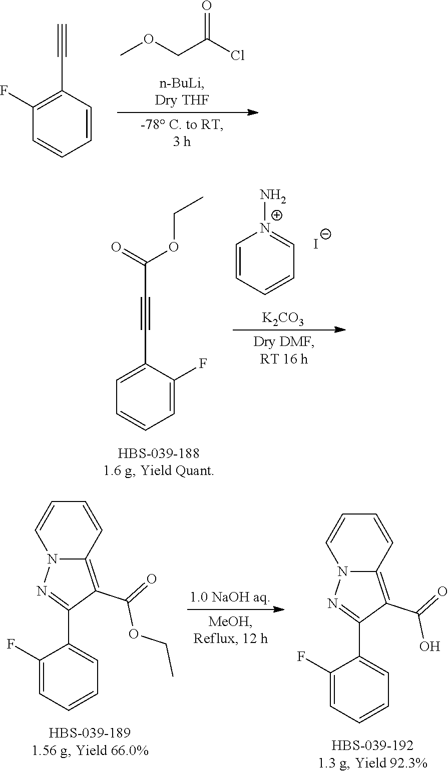

Step 1: Synthesis of HBS-039-188: 1-Ethynyl-2-fluorobenzene (1.0 g, 8.33 mmol) was dissolved in anhydrous THF (10.0 mL). The n-BuLi (10.4 mL, 16.65 mmol) was added at −78° C. temperature and rxn mixture was stirred at −78° C. temperature for 1 h. The ethyl chloroformate (3.8 mL, 40.0 mmol) was added at −78° C. temperature and rxn mixture was gradually warmed to ambient temperature. The LCMS data shows product formation m/z 193.0. The rxn mixture was diluted with aq. NH₄Cl solution and product was extracted with ethyl acetate. The combined ethyl acetate layer was separated and dried over anhydrous sodium sulfate. The evaporation of solvent gave crude product. The 1.6 g of crude product was obtained (Yield Quant.). MS (ESI) mass calcd. for C₁₁H₉FO₂, 192.19; m/z found 193.0 [M+H]⁺.

Step 2: Synthesis of HBS-039-189: Compound HBS-039-188 (1.6 g, 8.33 mmol) and 1-Aminopyridinium iodide (1.85 g, 8.33 mmol) were dissolved in anhydrous DMF (15.0 mL). The anhydrous K₂CO₃ (2.88 g, 20.81 mmol) was added, and reaction mixture was stirred at ambient temperature for 16 h. The LCMS data shows product formation m/z 285.1. The reaction mixture was diluted with water and the product was extracted with ethyl acetate. The combined ethyl acetate layer was separated and dried over anhydrous sodium sulfate. The evaporation of solvent gave crude product. The crude product was purified by column chromatography, Mobile Phase: EtOAc:Hexane, gradient. The 1.56 g of product was obtained (Yield 66.0%). MS (ESI) mass calcd. for C₁₆H₁₃FN₂O₂, 284.29; m/z found 285.1 [M+H]⁺.

Step 3: Synthesis of HBS-039-192: Compound HBS-039-189 (1.56 g, 5.5 mmol) was dissolved in MeOH (10.0 mL). The 1.0 N aq. NaOH solution (16.5 mL, 16.5 mmol) was added, and reaction mixture was refluxed for 12 h. LCMS shows product formation m/z 257.0. The reaction mixture was concentrated under reduced pressure. The solid was dissolved in water and acidified with 2.0 M aq. HCl solution (pH=5). The ppts were filtered and washed with water (5.0 mL×3) to obtain 1.3 g of solid product (Yield 92.3%). MS (ESI) mass calcd. for C₁₄H₉FN₂O₂, 256.23; m/z found 257.0 [M+H]⁺.

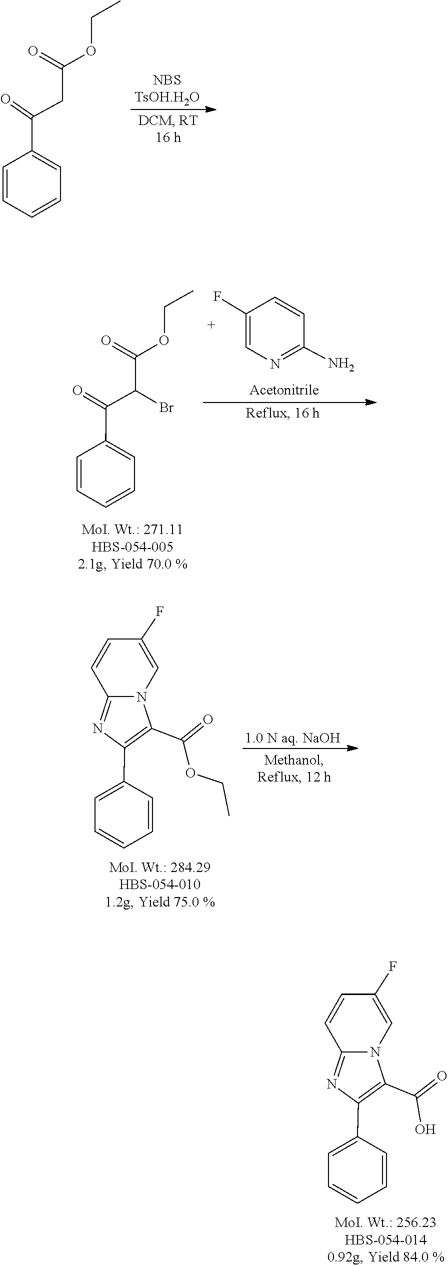

Step 1: Synthesis of HBS-054-005: The ethyl benzoylacetate (2.13 g, 11.1 mmol) was dissolved in DCM (20.0 mL). The NBS (1.8 g, 11.1 mmol) and TsOH·H₂O (0.38 g, 2.0 mmol) were added and reaction mixture was stirred at ambient temperature for 24 h. LCMS shows product formation m/z 272.0. The reaction mixture was diluted with water and the product was extracted with ethyl acetate. The ethyl acetate layer was separated and dried over anhydrous Na₂SO₄. The evaporation of solvent gave crude product. The crude product was purified by combi-flash system, Mobile phase: EtOAc:Hexane gradient. The 2.1 g of product was obtained (Yield 70.0%). MS (ESI) mass calcd. for C₁₁H₁₁BrO₃, 271.1; m/z found 272.0 [M+H]⁺.

Step 2: Synthesis of HBS-054-010: Compound HBS-054-005 (1.5 g, 5.56 mmol) was dissolved in anhydrous acetonitrile (20.0 mL). The 2-amino-5-fluoropyridine (1.9 g, 16.67 mmol) was added and reaction mixture was stirred at 80° C. for 16 h. LCMS shows product formation m/z 285.0. The reaction mixture was concentrated under reduced pressure to yield crude product. The crude product was purified by combi-flash system, Mobile phase: EtOAc:Hexane gradient. The 1.2 g of product was obtained (Yield 75.0%). MS (ESI) mass calcd. for C₁₆H₁₃FN₂O₂, 284.29; m/z found 285.0 [M+H]⁺.

Step 3: Synthesis of HBS-054-014: Compound HBS-054-010 (1.2 g, 4.22 mmol) was dissolved in MeOH (12.0 mL). The 1.0 N aq. NaOH solution (8.44 mL, 8.5 mmol) was added, and reaction mixture was stirred at 60° C. temperature for 12 h. LCMS shows product formation m/z 257.0. The reaction mixture was concentrated under reduced pressure. The solid was dissolved in water and acidified with 2.0 M aq. HCl solution (pH=5). The ppts were filtered and washed with water (5.0 mL×3) to obtain 0.92 g of solid product (Yield 84.0%). MS (ESI) mass calcd. for C₁₄H₉FN₂O₂, 256.23; m/z found 257.0 [M+H]⁺.

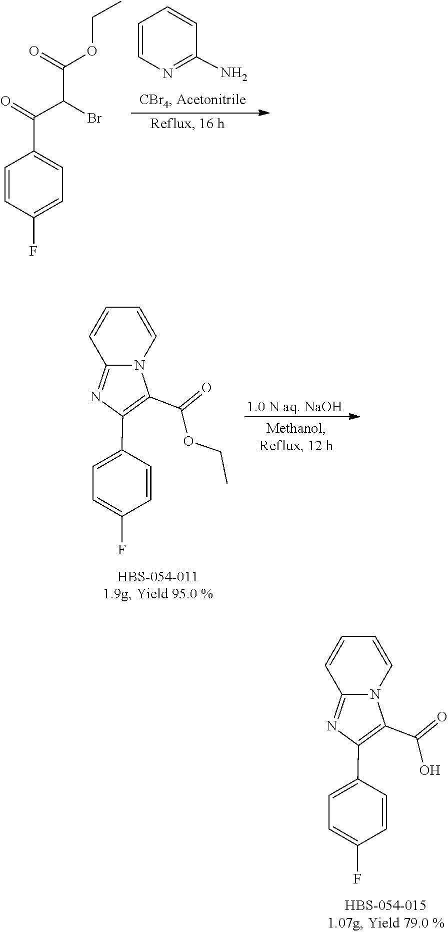

Step 1: Synthesis of HBS-054-011: Ethyl 3-(4-fluorophenyl)-3-oxopropanoate (1.5 g, 7.14 mmol) was dissolved in anhydrous acetonitrile (20.0 mL). The 2-amino-pyridine (2.0 g, 21.4 mmol) was added followed by CBr₄ (4.7 g, 14.27 mmol) and reaction mixture was stirred at 80° C. for 16 h. LCMS shows product formation m/z 285.0. The reaction mixture was concentrated under reduced pressure to yield crude product. The crude product was purified by combi-flash system, Mobile phase: EtOAc:Hexane gradient. The 1.9 g of product was obtained (Yield 95.0%). MS (ESI) mass calcd. for C₁₆H₁₃FN₂O₂, 284.29; m/z found 285.0 [M+H]⁺.

Step 2: Synthesis of HBS-054-015: Compound HBS-054-011 (1.5 g, 5.28 mmol) was dissolved in MeOH (15.0 mL). The 1.0 N aq. NaOH solution (10.6 mL, 10.56 mmol) was added, and reaction mixture was stirred at 60° C. temperature for 12 h. LCMS shows product formation m/z 257.0. The reaction mixture was concentrated under reduced pressure. The solid was dissolved in water and acidified with 2.0 M aq. HCl solution (pH=5). The ppts were filtered and washed with water (5.0 mL×3) to obtain 1.07 g of solid product (Yield 79.0%). MS (ESI) mass calcd. for C₁₄H₉FN₂O₂, 256.23; m/z found 257.0 [M+H]⁺.

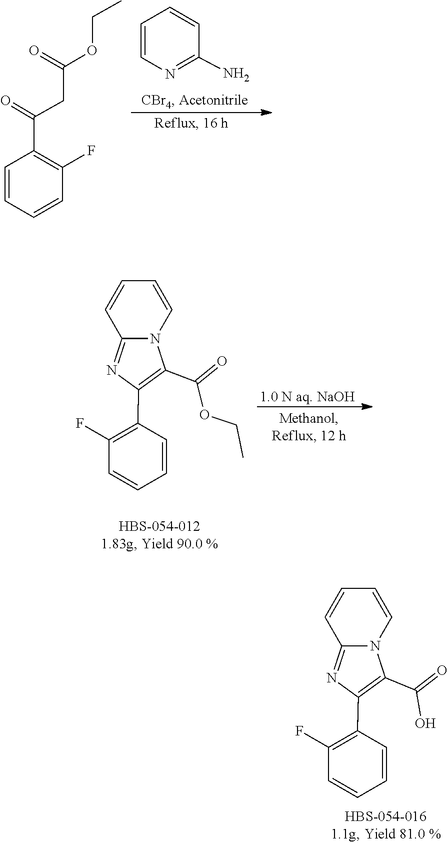

Step 1: Synthesis of HBS-054-012: Ethyl 3-(2-fluorophenyl)-3-oxopropanoate (1.5 g, 7.14 mmol) was dissolved in anhydrous acetonitrile (20.0 mL). The 2-amino-pyridine (2.0 g, 21.4 mmol) was added followed by CBr₄ (4.7 g, 14.27 mmol) and reaction mixture was stirred at 80° C. for 4 h. LCMS shows product formation m/z 285.0. The reaction mixture was concentrated under reduced pressure to yield crude product. The crude product was purified by combi-flash system, Mobile phase: EtOAc:Hexane gradient. The 1.83 g of product was obtained (Yield 90.0%). MS (ESI) mass calcd. for C₁₆H₁₃FN₂O₂, 284.29; m/z found 285.0 [M+H]⁺.

Step 2: Synthesis of HBS-054-016: Compound HBS-054-012 (1.5 g, 5.28 mmol) was dissolved in MeOH (15.0 mL). The 1.0 N aq. NaOH solution (10.6 mL, 10.56 mmol) was added, and reaction mixture was stirred at 60° C. temperature for 12 h. LCMS shows product formation m/z 257.0. The reaction mixture was concentrated under reduced pressure. The solid was dissolved in water and acidified with 2.0 M aq. HCl solution (pH=5). The ppts were filtered and washed with water (5.0 mL×3) to obtain 1.1 g of solid product (Yield 81.0%). MS (ESI) mass calcd. for C₁₄H₉FN₂O₂, 256.23; m/z found 257.0 [M+H]⁺.

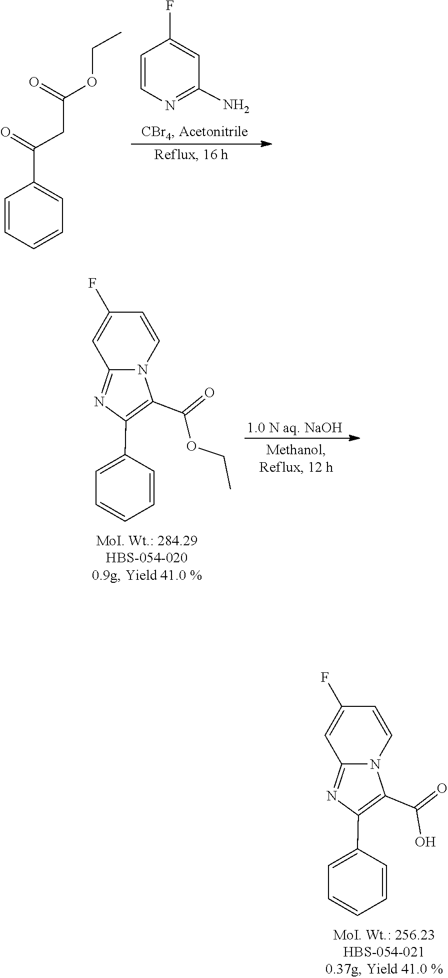

Step 1: Synthesis of HBS-054-020: Ethyl benzoylacetate (1.5 g, 7.81 mmol) was dissolved in anhydrous acetonitrile (20.0 mL). The 2-amino-4-fluoropyridine (2.6 g, 23.4 mmol) was added followed by CBr₄ (5.2 g, 15.6 mmol) and reaction mixture was stirred at 80° C. for 16 h. LCMS shows product formation m/z 285.0. The reaction mixture was concentrated under reduced pressure to yield crude product. The crude product was purified by combi-flash system, Mobile phase: EtOAc:Hexane gradient. The 0.9 g of product was obtained (Yield 41.0%). MS (ESI) mass calcd. for C₁₆H₁₃FN₂O₂, 284.29; m/z found 285.0 [M+H]⁺.

Step 2: Synthesis of HBS-054-021: Compound HBS-054-020 (0.9 g, 3.17 mmol) was dissolved in MeOH (10.0 mL). The 1.0 N aq. NaOH solution (6.3 mL, 6.34 mmol) was added, and reaction mixture was stirred at 60° C. temperature for 12 h. LCMS shows product formation m/z 257.0. The reaction mixture was concentrated under reduced pressure. The solid was dissolved in water and acidified with 2.0 M aq. HCl solution (pH=5). The ppts were filtered and washed with water (5.0 mL×3) to obtain 0.37 g of solid product (Yield 41.0%). MS (ESI) mass calcd. for C₁₄H₉FN₂O₂, 256.23; m/z found 257.0 [M+H]⁺.

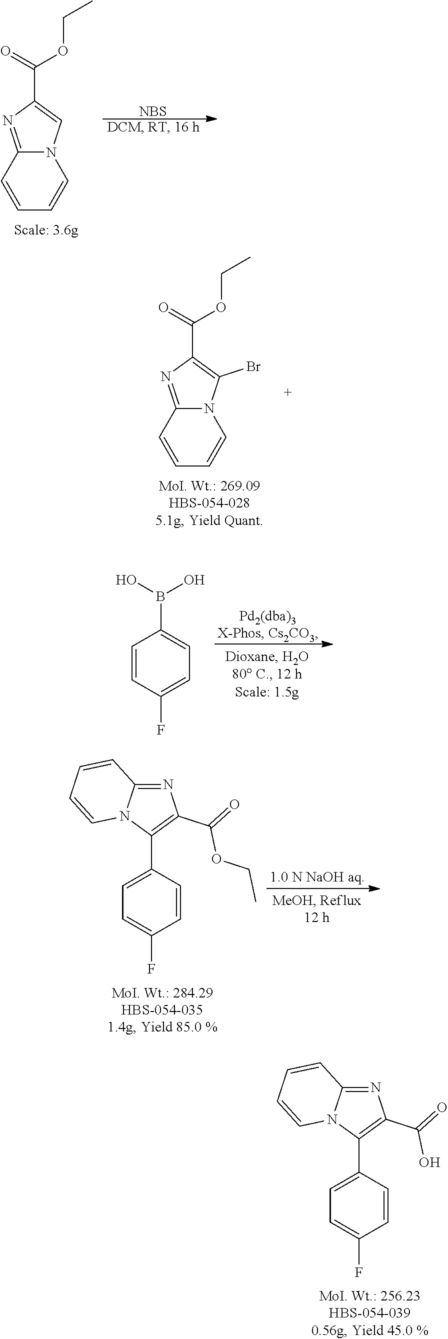 [SMILES: CCOC(=O)c1cn2ccccc2n1 ; "NBS" ; CCOC(=O)c1nc2ccccn2c1Br ; OB(O)c1ccc(F)cc1 ; CCOC(=O)c1nc2ccccn2c1-c1ccc(F)cc1 ; O=C(O)c1nc2ccccn2c1-c1ccc(F)cc1]

Step 1: Synthesis of HBS-054-028: Ethyl imidazo[1,2-a]pyridine-2-carboxylate (3.6 g, 18.94 mmol) was dissolved in DCM (80.0 mL). The NBS (3.4 g, 18.94 mmol) was added and rxn mixture was stirred at ambient temperature for 16 h. The LCMS data shows product formation m/z 270.0. The rxn mixture was concentrated under reduced pressure to obtain the crude product. The crude product was purified by ISCO combi-flash chromatography system, Mobile phase: EtOAc:Hexane gradient. The 5.1 g of product was obtained (Yield Quant.). MS (ESI) mass calcd. for C₁₀H₉BrN₂O₂, 269.1; m/z found 270.0 [M+H]⁺.

Step 2: Synthesis of HBS-054-035: Compound HBS-054-028 (1.5 g, 5.6 mmol) and 4-fluorophenylboronic acid (1.2 g, 8.4 mmol) were dissolved in mixture of Dioxane/water (24.0:6.0 v/v mL). The anhydrous Cs₂CO₃ (3.8 g, 11.75 mmol) was added followed by Pd₂(dba)₃ (0.26 g, 0.28 mmol) and X-Phos (0.4 g, 0.84 mmol). The rxn mixture was stirred at 80° C. temperature under N₂ atm. for 12 h. The LCMS data shows product formation m/z 285.0. The rxn mixture was filtered over celite bed and washed with ethyl acetate. The filtrate was concentrated under reduced pressure to obtain the crude product. The crude product was purified by ISCO combi-flash chromatography system, Mobile phase: EtOAc:Hexane gradient. The 1.4 g of product was obtained (Yield 85.0%). MS (ESI) mass calcd. for C₁₆H₁₃FN₂O₂, 284.29; m/z found 285.0 [M+H]⁺.

Step 3: Synthesis of HBS-054-039: Compound HBS-054-035 (1.4 g, 4.93 mmol) was dissolved in MeOH (15.0 mL). The 1.0 N aq. solution of NaOH (10.0 mL, 9.86 mmol) was added and rxn mixture was refluxed for 12 h. The LCMS data shows product formation m/z 257.0. The rxn mixture was concentrated under reduced pressure and diluted with water. The aq. layer was acidified with 2.0 M aq. HCl solution (pH=5). The ppts were filtered and washed with water (5.0 mL×3) to obtain 0.56 g of solid product (Yield 45.0%). MS (ESI) mass calcd. for C₁₄H₉FN₂O₂, 256.23; m/z found 257.0 [M+H]⁺.

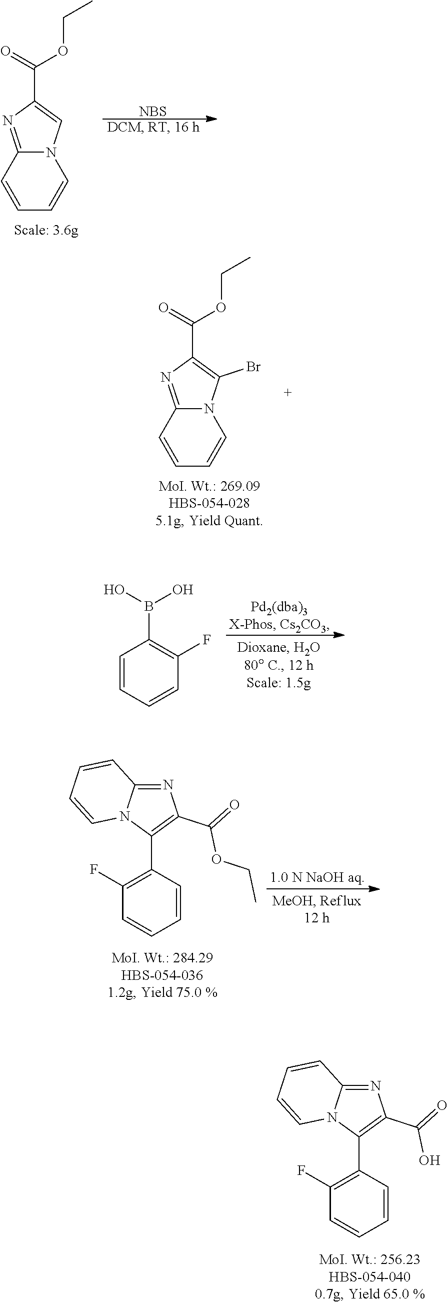 [SMILES: CCOC(=O)c1cn2ccccc2n1 ; "NBS" ; CCOC(=O)c1nc2ccccn2c1Br ; OB(O)c1ccccc1F ; CCOC(=O)c1nc2ccccn2c1-c1ccccc1F ; O=C(O)c1nc2ccccn2c1-c1ccccc1F]

Step 1: Synthesis of HBS-054-028: Ethyl imidazo[1,2-a]pyridine-2-carboxylate (3.6 g, 18.94 mmol) was dissolved in DCM (80.0 mL). The NBS (3.4 g, 18.94 mmol) was added and rxn mixture was stirred at ambient temperature for 16 h. The LCMS data shows product formation m/z 270.0. The rxn mixture was concentrated under reduced pressure to obtain the crude product. The crude product was purified by ISCO combi-flash chromatography system, Mobile phase: EtOAc:Hexane gradient. The 5.1 g of product was obtained (Yield Quant.). MS (ESI) mass calcd. for C₁₀H₉BrN₂O₂, 269.1; m/z found 270.0 [M+H]⁺.

Step 2: Synthesis of HBS-054-036: Compound HBS-054-028 (1.5 g, 5.6 mmol) and 2-fluorophenylboronic acid (1.2 g, 8.4 mmol) were dissolved in mixture of Dioxane/water (24.0:6.0 v/v mL). The anhydrous Cs₂CO₃ (3.8 g, 11.75 mmol) was added followed by Pd₂(dba)₃ (0.26 g, 0.28 mmol) and X-Phos (0.4 g, 0.84 mmol). The rxn mixture was stirred at 80° C. temperature under N₂ atm. for 12 h. The LCMS data shows product formation m/z 285.0. The rxn mixture was filtered over celite bed and washed with ethyl acetate. The filtrate was concentrated under reduced pressure to obtain the crude product. The crude product was purified by ISCO combi-flash chromatography system, Mobile phase: EtOAc:Hexane gradient. The 1.2 g of product was obtained (Yield 75.0%). MS (ESI) mass calcd. for C₁₆H₁₃FN₂O₂, 284.29; m/z found 285.0 [M+H]⁺.

Step 3: Synthesis of HBS-054-040: Compound HBS-054-036 (1.2 g, 4.22 mmol) was dissolved in MeOH (15.0 mL). The 1.0 N aq. solution of NaOH (8.4 mL, 8.44 mmol) was added and rxn mixture was refluxed for 12 h. The LCMS data shows product formation m/z 257.0. The rxn mixture was concentrated under reduced pressure and diluted with water. The aq. layer was acidified with 2.0 M aq. HCl solution (pH=5). The ppts were filtered and washed with water (5.0 mL×3) to obtain 0.7 g of solid product (Yield 65.0%). MS (ESI) mass calcd. for C₁₄H₉FN₂O₂, 256.23; m/z found 257.0 [M+H]⁺.

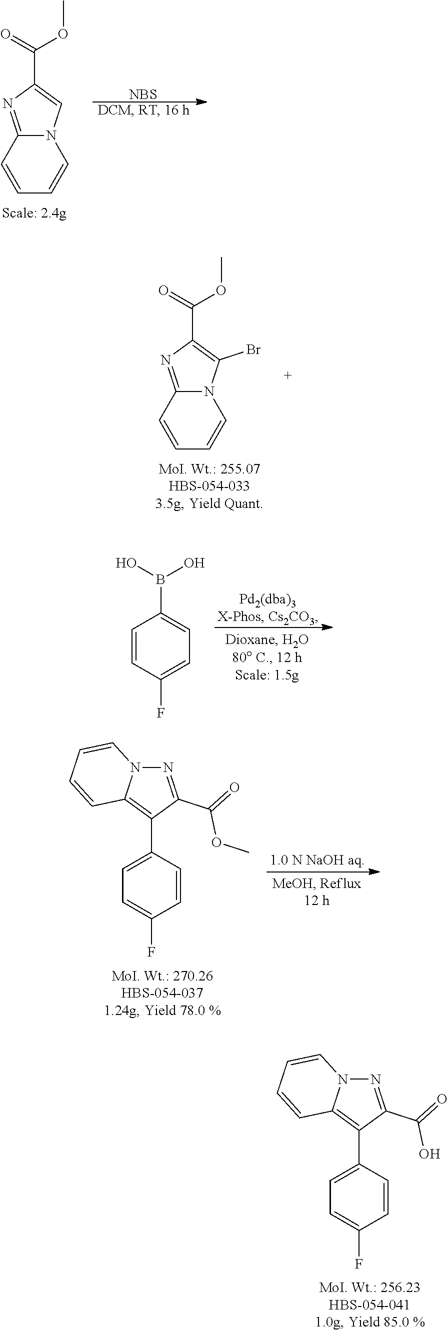 [SMILES: COC(=O)c1cn2ccccc2n1 ; "NBS" ; COC(=O)c1nc2ccccn2c1Br ; OB(O)c1ccc(F)cc1 ; COC(=O)c1nn2ccccc2c1-c1ccc(F)cc1 ; O=C(O)c1nn2ccccc2c1-c1ccc(F)cc1]

Step 1: Synthesis of HBS-054-033: Pyrazolo[1,5-a]pyridine-2-carboxylic acid methyl ester (2.4 g, 13.6 mmol) was dissolved in DCM (54.0 mL). The NBS (2.5 g, 14.3 mmol) was added and rxn mixture was stirred at ambient temperature for 16 h. The LCMS data shows product formation m/z 256.0. The rxn mixture was concentrated under reduced pressure to obtain the crude product. The crude product was purified by ISCO combi-flash chromatography system, Mobile phase: EtOAc:Hexane gradient. The 3.5 g of product was obtained (Yield Quant.). MS (ESI) mass calcd. for C₉H₇BrN₂O₂, 255.07; m/z found 256.0 [M+H]⁺.

Step 2: Synthesis of HBS-054-037: Compound HBS-054-033 (1.5 g, 5.9 mmol) and 4-fluorophenylboronic acid (1.2 g, 8.86 mmol) were dissolved in mixture of Dioxane/water (24.0:6.0 v/v mL). The anhydrous Cs₂CO₃ (4.0 g, 12.39 mmol) was added followed by Pd₂(dba)₃ (0.27 g, 0.29 mmol) and X-Phos (0.4 g, 0.88 mmol). The rxn mixture was stirred at 80° C. temperature under N₂ atm. for 12 h. The LCMS data shows product formation m/z 271.0. The rxn mixture was filtered over celite bed and washed with ethyl acetate. The filtrate was concentrated under reduced pressure to obtain the crude product. The crude product was purified by ISCO combi-flash chromatography system, Mobile phase: EtOAc:Hexane gradient. The 1.24 g of product was obtained (Yield 78.0%). MS (ESI) mass calcd. for C₁₅H₁₁FN₂O₂, 270.26; m/z found 271.0 [M+H]⁺.

Step 3: Synthesis of HBS-054-041: Compound HBS-054-037 (1.2 g, 4.59 mmol) was dissolved in MeOH (15.0 mL). The 1.0 N aq. solution of NaOH (9.2 mL, 9.18 mmol) was added and rxn mixture was refluxed for 12 h. The LCMS data shows product formation m/z 257.0. The rxn mixture was concentrated under reduced pressure and diluted with water. The aq. layer was acidified with 2.0 M aq. HCl solution (pH=5). The ppts were filtered and washed with water (5.0 mL×3) to obtain 1.0 g of solid product (Yield 85.0%). MS (ESI) mass calcd. for C₁₄H₉FN₂O₂, 256.23; m/z found 257.0 [M+H]⁺.

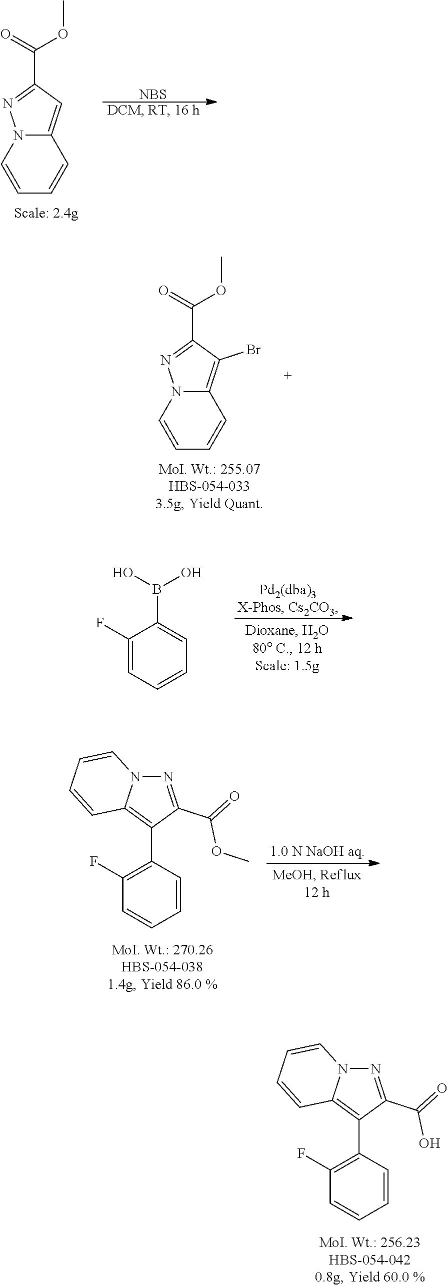 [SMILES: COC(=O)c1cc2ccccn2n1 ; "NBS" ; COC(=O)c1nn2ccccc2c1Br ; OB(O)c1ccccc1F ; COC(=O)c1nn2ccccc2c1-c1ccccc1F ; O=C(O)c1nn2ccccc2c1-c1ccccc1F]

Step 1: Synthesis of HBS-054-033: Pyrazolo[1,5-a]pyridine-2-carboxylic acid methyl ester (2.4 g, 13.6 mmol) was dissolved in DCM (54.0 mL). The NBS (2.5 g, 14.3 mmol) was added and rxn mixture was stirred at ambient temperature for 16 h. The LCMS data shows product formation m/z 256.0. The rxn mixture was concentrated under reduced pressure to obtain the crude product. The crude product was purified by ISCO combi-flash chromatography system, Mobile phase: EtOAc:Hexane gradient. The 3.5 g of product was obtained (Yield Quant.). MS (ESI) mass calcd. for C₉H₇BrN₂O₂, 255.07; m/z found 256.0 [M+H]⁺.

Step 2: Synthesis of HBS-054-038: Compound HBS-054-033 (1.5 g, 5.9 mmol) and 2-fluorophenylboronic acid (1.2 g, 8.86 mmol) were dissolved in mixture of Dioxane/water (24.0:6.0 v/v mL). The anhydrous Cs₂CO₃ (4.0 g, 12.39 mmol) was added followed by Pd₂(dba)₃ (0.27 g, 0.29 mmol) and X-Phos (0.4 g, 0.88 mmol). The rxn mixture was stirred at 80° C. temperature under N₂ atm. for 12 h. The LCMS data shows product formation m/z 271.0. The rxn mixture was filtered over celite bed and washed with ethyl acetate. The filtrate was concentrated under reduced pressure to obtain the crude product. The crude product was purified by ISCO combi-flash chromatography system, Mobile phase: EtOAc:Hexane gradient. The 1.4 g of product was obtained (Yield 86.0%). MS (ESI) mass calcd. for C₁₅H₁₁FN₂O₂, 270.26; m/z found 271.0 [M+H]⁺.

Step 3: Synthesis of HBS-054-042: Compound HBS-054-038 (1.4 g, 5.18 mmol) was dissolved in MeOH (15.0 mL). The 1.0 N aq. solution of NaOH (10.4 mL, 10.36 mmol) was added and rxn mixture was refluxed for 12 h. The LCMS data shows product formation m/z 257.0. The rxn mixture was concentrated under reduced pressure and diluted with water. The aq. layer was acidified with 2.0 M aq. HCl solution (pH=5). The ppts were filtered and washed with water (5.0 mL×3) to obtain 1.0 g of solid product (Yield 85.0%). MS (ESI) mass calcd. for C₁₄H₉FN₂O₂, 256.23; m/z found 257.0 [M+H]⁺.

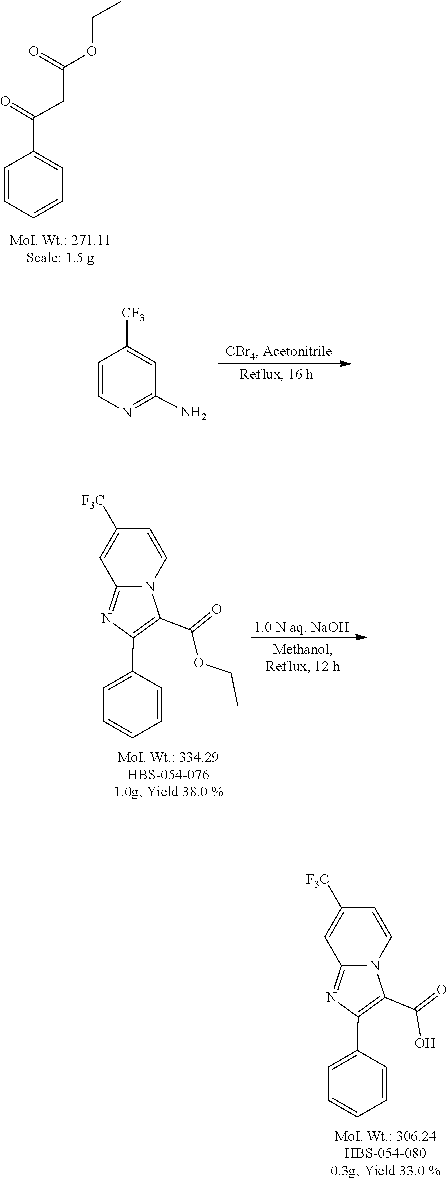 [SMILES: CCOC(=O)CC(=O)c1ccccc1 ; Nc1cc(C(F)(F)F)ccn1 ; CCOC(=O)c1c(-c2ccccc2)nc2cc(C(F)(F)F)ccn12 ; O=C(O)c1c(-c2ccccc2)nc2cc(C(F)(F)F)ccn12]

Step 1: Synthesis of HBS-054-076: Ethyl benzoylacetate (1.5 g, 7.81 mmol) was dissolved in anhydrous acetonitrile (20.0 mL). The 2-amino-4-(trifluoromethyl)pyridine (3.8 g, 23.4 mmol) was added followed by CBr₄ (5.2 g, 15.6 mmol) and reaction mixture was stirred at 80° C. for 16 h. LCMS shows product formation m/z 335.0. The reaction mixture was concentrated under reduced pressure to yield crude product. The crude product was purified by combi-flash system, Mobile phase: EtOAc:Hexane gradient. The 1.0 g of product was obtained (Yield 38.0%). MS (ESI) mass calcd. for C₁₇H₁₃F₃N₂O₂, 334.29; m/z found 335.0 [M+H]⁺.

Step 2: Synthesis of HBS-054-080: Compound HBS-054-076 (1.0 g, 2.99 mmol) was dissolved in MeOH (10.0 mL). The 1.0 N aq. NaOH solution (6.0 mL, 6.0 mmol) was added, and reaction mixture was refluxed for 12 h. LCMS shows product formation m/z 307.0. The reaction mixture was concentrated under reduced pressure. The solid was dissolved in water and acidified with 2.0 M aq. HCl solution (pH=5). The ppts were filtered and washed with water (5.0 mL×3) to obtain 0.3 g of solid product (Yield 33.0%). MS (ESI) mass calcd. for C₁₅H₉F₃N₂O₂, 306.24; m/z found 307.0 [M+H]⁺.

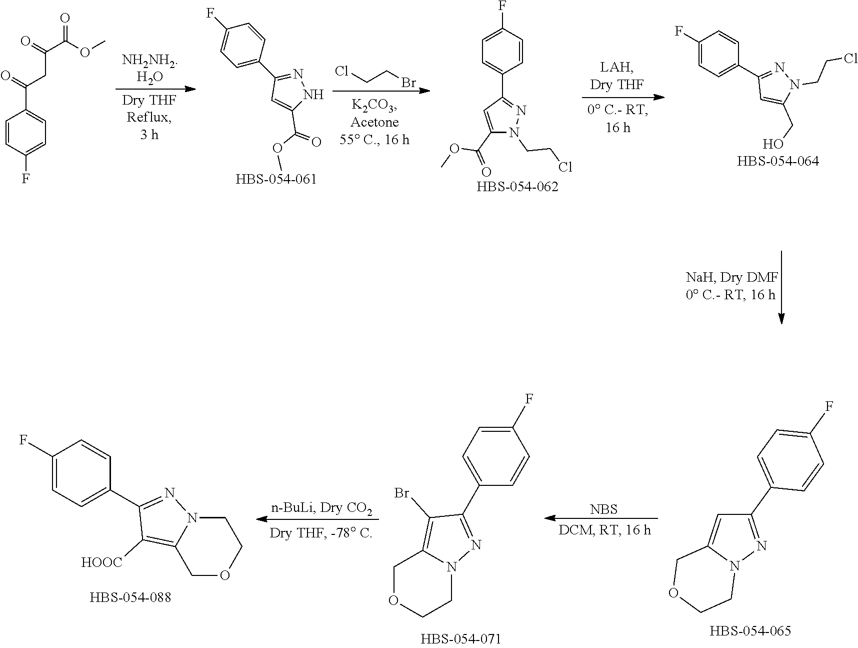

Step 1: Synthesis of HBS-054-061: Methyl 4-(4-fluorophenyl)-2,4-dioxobutanoate (2.2 g, 10.68 mmol) was dissolved in anhydrous THF (40.0 mL). The hydrazine monohydrate (0.56 g, 11.21 mmol) was added. The reaction mixture was heated at reflux for 3 h. LCMS data shows desired product formation m/z 221.0. The reaction mixture was concentrated under reduced pressure to obtain the crude product. The crude product was purified by column chromatography, Mobile Phase: EtOAc:Hexane, gradient. The 1.2 g of product was isolated (Yield 51.0%). MS (ESI) mass calcd. for C₁₁H₉FN₂O₂, 220.2; m/z found 221.0 [M+H]⁺.

Step 2: Synthesis of HBS-054-062: Compound HBS-054-061 (0.64 g, 2.91 mmol) was dissolved in Acetone (15.0 mL). The K₂CO₃ (0.8 g, 5.82 mmol) was added, followed by 1-Bromo-2-Chloro-ethane (0.5 g, 3.49 mmol). The reaction mixture was heated at 55° C. for 16 h. LCMS data shows desired product formation m/z 283.0 and minor amount of side product formation. The reaction mixture was filtered and the solid was washed with ethyl acetate. The filtrate was concentrated under reduced pressure to obtain crude product. The crude product was purified by column chromatography, Mobile Phase: EtOAc:Hexane, gradient. The 0.7 g of product was isolated (Yield 85.0%). MS (ESI) mass calcd. for C₁₃H₁₂ClFN₂O₂, 282.7; m/z found 283.0 [M+H]⁺.

Step 3: Synthesis of HBS-054-064: Compound HBS-054-062 (0.7 g, 2.48 mmol) was dissolved in dry THF (10.0 mL). The 2.0 M LAH solution in THF (1.24 mL, 2.48 mmol) was added, under ice cooling bath. The reaction mixture was gradually warmed to room temperature and stirred for 16 h. LCMS data shows desired product formation m/z 255.0. The reaction mixture was quenched with 1.0 N aq. NaOH solution and diluted with ethyl acetate (10.0 mL). The reaction mixture was filtered through celite bed and washed with ethyl acetate (10.0 mL×3). The organic layer was separated and washed with water followed by brine. The organic layer was dried over anhydrous sodium sulfate. The evaporation of solvent gave crude product 0.6 g (Yield 95.0%). MS (ESI) mass calcd. for C₁₂H₁₂ClFN₂O, 254.69; m/z found 255.0 [M+H]⁺.

Step 4: Synthesis of HBS-054-065: Compound HBS-054-064 (0.6 g, 2.36 mmol) was dissolved in dry DMF (5.0 mL). The NaH (0.11 g, 4.72 mmol) was added under ice cooling. The reaction mixture was gradually warmed to room temperature and stirred for 16 h. LCMS data shows desired product formation m/z 219.0. The reaction mixture was diluted with water and the product was extracted with ethyl acetate. The combined ethyl acetate layer was separated and dried over anhydrous sodium sulfate. The evaporation of solvent gave crude product. The crude product was purified by column chromatography, Mobile Phase: EtOAc:Hexane, gradient. The 0.46 g of solid product was obtained (Yield 88.0%). MS (ESI) mass calcd. for C₁₂H₁₁FN₂O, 218.23; m/z found 219.0 [M+H]⁺.

Step 5: Synthesis of HBS-054-071: Compound HBS-054-065 (0.46 g, 2.11 mmol) was dissolved in DCM (7.0 mL). The NBS (0.41 g, 2.32 mmol) was added, and reaction mixture was stirred at room temperature for 16 h. LCMS data shows desired product formation m/z 298.0. The evaporation of solvent gave crude product. The crude product was purified by column chromatography, Mobile Phase: EtOAc:Hexane, gradient. The 0.25 g of product was obtained (Yield 40.0%). MS (ESI) mass calcd. for C₁₂H₁₀BrFN₂O, 297.12; m/z found 298.0 [M+H]⁺.

Step 6: Synthesis of HBS-054-088: Compound HBS-054-071 (1.15 g, 3.85 mmol) was dissolved in anhydrous THF (20.0 mL) under N₂ atm. The reaction mixture was cooled at −78.0° C. temperature and 1.6 M n-BuLi in Hexane (4.81 mL, 7.7 mmol) was added into the reaction mixture. The reaction mixture was stirred at −78.0° C. temperature for 30.0 min. The dry CO₂ gas bubbled through the reaction mixture at −65° C. and the reaction mixture gradually warmed at room temperature. The LCMS data shows desired product formation m/z 263.0, debrominated side product and some unknown product formation. The reaction mixture was quenched by water and extracted with ethyl acetate. The ethyl acetate layer was separated and debrominated product was recovered. The aq. Layer was acidified with 2 M HCl solution to obtain the ppts. The ppts were filtered and dried to obtain 0.6 g of solid product (Yield 60.0%). MS (ESI) mass calcd. for C₁₃H₁₁FN₂O₃, 262.24; m/z found 263.0 [M+H]⁺.

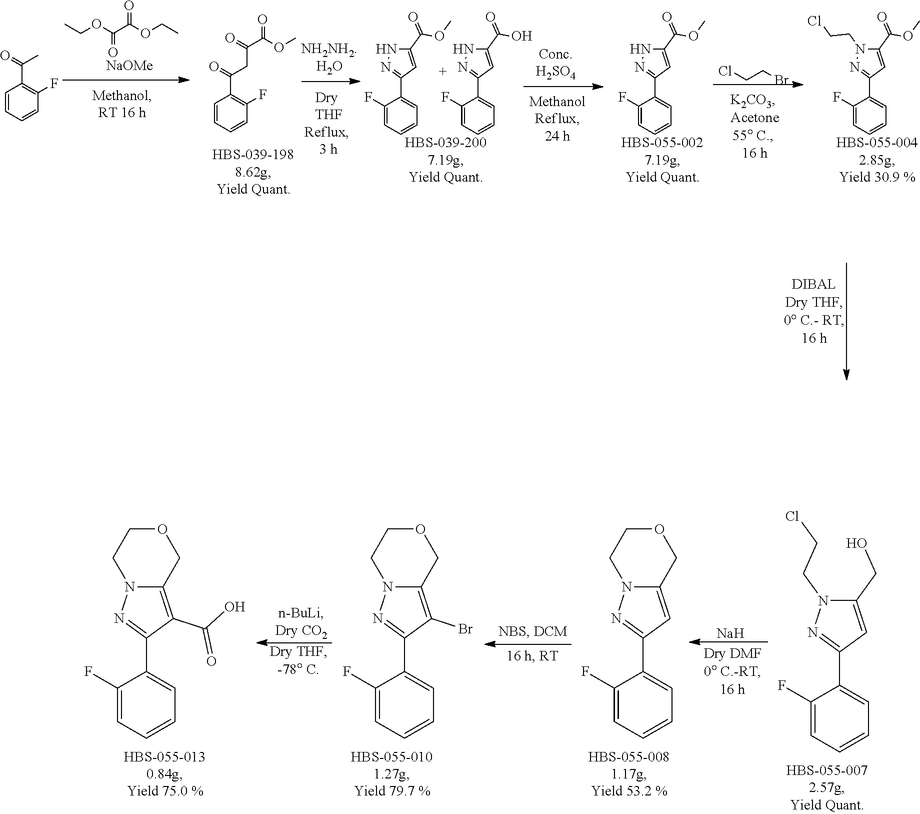

Step 1: Synthesis of HBS-039-198: 2-Fluoroacetophenone (5.0 g, 36.19 mmol) was added dropwise in NaOMe solution (1.25 g Na in 25.0 mL of Methanol). The rxn mixture was stirred at ambient temperature for 30.0 min. The Diethyl oxalate (5.81 g, 39.81 mmol) solution in anhydrous Methanol (25.0 mL) was added and reaction mixture was stirred at ambient temperature for 16 h. LCMS data shows desired product formation m/z 225.0. The reaction mixture was concentrated under reduced pressure to obtain the crude product. The crude product was dissolved in cold water and acidified with 2.0 M aq. HCl solution. The ppts were filtered and dried to obtain 8.62 g of crude product. MS (ESI) mass calcd. for C₁₁H₉FO₄, 224.19; m/z found 225.0 [M+H]⁺.

Step 2: Synthesis of HBS-039-200: Compound HBS-039-198 (8.62 g, 36.19 mmol) was dissolved in IPA (100.0 mL). The hydrazine monohydrate (2.1 mL, 43.4 mmol) was added, and reaction mixture was heated at reflux for 3 h. LCMS data shows desired product formation m/z 221.0. and hydrolyzed side product m/z 207.0. The reaction mixture was cooled at ambient temperature to obtain ppts. The ppts were filtered to obtain 7.19 g of crude product. (Yield 90.2%). MS (ESI) mass calcd. for C₁₁H₉FN₂O₂, 220.2; m/z found 221.0 [M+H]⁺ and mass calcd. for C₁₀H₇FN₂O₂, 206.17; m/z found 207.0 [M+H]⁺.

Step 3: Synthesis of HBS-055-002: Compound HBS-039-200 crude (7.19 g, 32.65 mmol) was dissolved in anhydrous Methanol (100.0 mL). The conc. Sulfuric acid (4.0 mL) was added, and reaction mixture was heated at reflux for 24 h. LCMS data shows desired product formation m/z 221.0. The reaction mixture was cooled at ambient temperature and neutralized with aq. saturated solution of sodium bicarbonate to obtain ppts. The ppts were filtered and dried to obtain 7.19 g of solid product. (Yield Quant.). MS (ESI) mass calcd. for C₁₁H₉FN₂O₂, 220.2; m/z found 221.0 [M+H]⁺.

Step 4: Synthesis of HBS-055-004: Compound HBS-055-002 (7.19 g, 32.65 mmol) was dissolved in Acetone (100.0 mL). The K₂CO₃ (13.53 g, 97.95 mmol) was added, followed by 1-Bromo-2-Chloro-ethane (13.5 mL, 163.3 mmol). The reaction mixture was heated at 65° C. for 24 h. LCMS data shows desired product formation m/z 283.0. The reaction mixture was filtered and the solid was washed with ethyl acetate. The filtrate was concentrated under reduced pressure to obtain crude product. The crude product was purified by column chromatography, Mobile Phase: EtOAc:Hexane, gradient. The 2.85 g of product was isolated (Yield 30.9%). MS (ESI) mass calcd. for C₁₃H₁₂ClFN₂O₂, 282.7; m/z found 283.0 [M+H]⁺.

Step 5: Synthesis of HBS-055-007: Compound HBS-055-004 (2.85 g, 10.1 mmol) was dissolved in dry THF (25.0 mL). The reaction mixture was cooled at 0° C. temperature in the ice bath. The 1.0 M DIBAL solution in Hexane (25.2 mL, 25.2 mmol) was added. The reaction mixture was gradually warmed to room temperature and stirred for 16 h. LCMS data shows desired product formation m/z 255.1. The reaction mixture was quenched with aq. NH₄Cl solution and diluted with ethyl acetate (100.0 mL). The reaction mixture was filtered through celite bed and washed with ethyl acetate. The organic layer was separated and washed with brine. The organic layer was dried over anhydrous sodium sulfate. The evaporation of solvent gave crude product 2.57 g (Yield Quant.). MS (ESI) mass calcd. for C₁₂H₁₂ClFN₂O, 254.69; m/z found 255.0 [M+H]⁺.

Step 6: Synthesis of HBS-055-008: Compound HBS-055-007 (2.57 g, 10.1 mmol) was dissolved in dry DMF (20.0 mL). The 60.0% NaH (0.81 g, 20.2 mmol) in mineral oil was added under ice cooling. The reaction mixture was gradually warmed to room temperature and stirred for 16 h. LCMS data shows desired product formation m/z 219.1. The reaction mixture was diluted with water and the product was extracted with ethyl acetate. The combined ethyl acetate layer was separated and dried over anhydrous sodium sulfate. The evaporation of solvent gave crude product. The crude product was purified by column chromatography, Mobile Phase: EtOAc:Hexane, gradient. The 1.17 g of solid product was obtained (Yield 53.2%). MS (ESI) mass calcd. for C₁₂H₁₁FN₂O, 218.23; m/z found 219.1 [M+H]⁺.

Step 7: Synthesis of HBS-055-010: Compound HBS-055-008 (1.17 g, 5.36 mmol) was dissolved in DCM (15.0 mL). The NBS (1.05 g, 5.9 mmol) was added, and reaction mixture was stirred at ambient temperature for 16 h. LCMS data shows desired product formation m/z 299.0. The evaporation of solvent gave crude product. The crude product was purified by column chromatography, Mobile Phase: EtOAc:Hexane, gradient. The 1.27 g of product was obtained (Yield 79.7%). MS (ESI) mass calcd. for C₁₂H₁₀BrFN₂O, 297.12; m/z found 299.0 [M+H]⁺.

Step 8: Synthesis of HBS-055-013: Compound HBS-055-010 (1.27 g, 4.27 mmol) was dissolved in anhydrous THF (15.0 mL) under N₂ atm. The reaction mixture was cooled at −78.0° C. temperature. The 1.6 M n-BuLi in Hexane (5.33 mL, 8.54 mmol) was added and reaction mixture was stirred at −78.0° C. temperature for 30.0 min. The dry CO₂ gas bubbled through the reaction mixture at −65° C. temperature and the reaction mixture gradually warmed at room temperature. The LCMS data shows desired product formation m/z 263.1, debrominated side product and some unknown product formation. The reaction mixture was quenched by water and extracted with ethyl acetate. The ethyl acetate layer was separated and debrominated product was recovered. The aq. Layer was acidified with 2 M HCl solution to obtain the ppts. The ppts were filtered and dried to obtain 0.84 g of solid product (Yield 75.0%). MS (ESI) mass calcd. for C₁₃H₁₁FN₂O₃, 262.24; m/z found 263.1 [M+H]⁺.

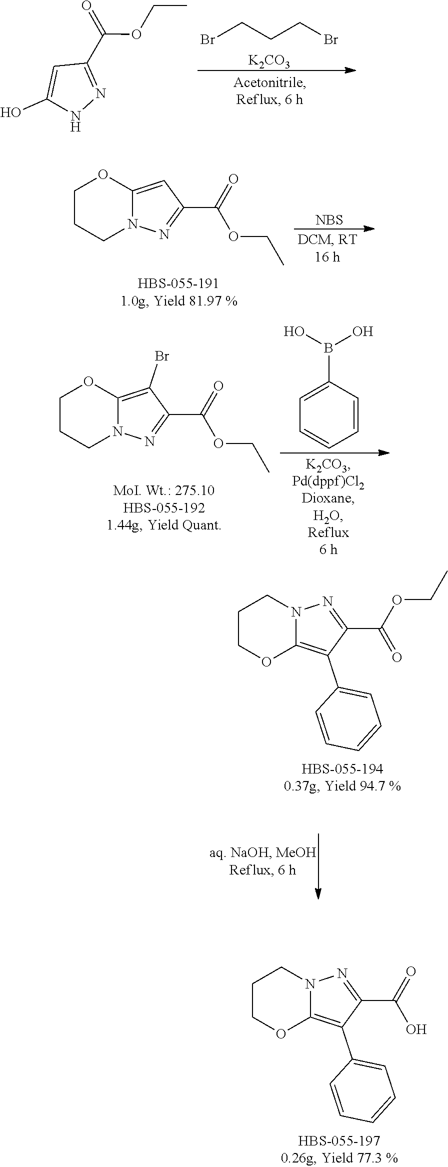

Step 1: Synthesis of HBS-055-191: Ethyl-5-hydroxy-1H-pyrazole-3-carboxylate (1.0 g, 6.40 mmol) was dissolved in anhydrous Acetonitrile (15.0 mL). The anhydrous K₂CO₃ (3.54 g, 25.62 mmol) was added, and reaction mixture was stirred at ambient temperature for 15.0 min. The 1,3-dibromo propane (0.72 mL, 7.05 mmol) was added, and reaction mixture was heated at reflux for 6 h. The LCMS data shows product formation m/z 197.1. The reaction mixture cooled at ambient temperature and filtered. The filtrate was concentrated under reduced pressure to obtain the crude product. The crude product was purified by ISCO combi-flash chromatography system, Mobile phase: EtOAc:Hexane gradient. The 1.03 g of product was obtained (Yield 81.97%). MS (ESI) mass calcd. for C₉H₁₂N₂O₃, 196.20; m/z found 197.1 [M+H]⁺.

Step 2: Synthesis of HBS-055-192: Compound HBS-055-191 (1.03 g, 5.25 mmol) was dissolved in DCM (15.0 mL). The NBS (0.93 g, 5.25 mmol) was added and rxn mixture was stirred at ambient temperature for 16 h. The LCMS data shows product formation m/z 275.0. The reaction mixture was diluted with water and the product was extracted with DCM. The combined DCM layer was separated and dried over anhydrous sodium sulfate. The evaporation of solvent gave crude product. The crude product was purified by ISCO combi-flash system, Mobile phase: EtOAc:Hexane gradient. The 1.44 g of product was obtained (Yield Quant.). MS (ESI) mass calcd. for C₉H₁₁BrN₂O₃, 275.1; m/z found 275.0 [M+H]⁺.

Step 3: Synthesis of HBS-055-194: Compound HBS-055-192 (0.4 g, 1.45 mmol) and Phenylboronic acid (0.27 g, 2.18 mmol) were dissolved in mixture of Dioxane/water (9:1 v/v mL). The anhydrous K₂CO₃ (0.6 g, 4.36 mmol) was added followed by Pd(dppf)Cl₂·DCM₂ (0.06 g, 0.073 mmol). The rxn mixture was stirred at 100° C. temperature under N₂ atm. for 6 h. The LCMS data shows product formation m/z 273.1. The rxn mixture was filtered over celite bed and washed with ethyl acetate. The filtrate was concentrated under reduced pressure to obtain the crude product. The crude product was purified by ISCO combi-flash chromatography system, Mobile phase: EtOAc:Hexane gradient. The 0.37 g of product was obtained (Yield 94.7%). MS (ESI) mass calcd. for C₁₅H₁₆N₂O₃, 272.3; m/z found 273.1 [M+H]⁺.

Step 4: Synthesis of HBS-055-197: Compound HBS-055-194 (0.37 g, 1.38 mmol) was dissolved in MeOH (5.0 mL). The 1.0 N aq. solution of NaOH (4.1 mL, 4.1 mmol) was added and rxn mixture was refluxed for 6 h. The LCMS data shows product formation m/z 245.1. The rxn mixture was concentrated under reduced pressure and diluted with water. The aq. layer was acidified with 2.0 M aq. HCl solution (pH=5). The ppts were filtered and washed with water (2.0 mL×3) to obtain 0.26 g of solid product (Yield 77.3%). MS (ESI) mass calcd. for C₁₃H₁₂N₂O₃, 244.25; m/z found 245.1 [M+H]⁺.

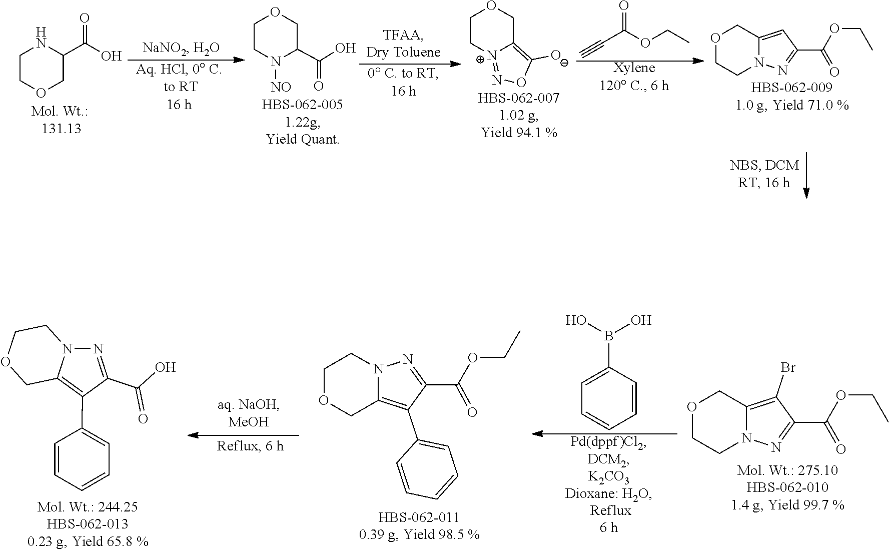

Step 1: Synthesis of HBS-062-005: Morpholine-3-carboxylic acid (1.0 g, 7.63 mmol) was dissolved in water (6.0 mL). Anhydrous NaNO₂ (0.79 g, 11.44 mmol) was added and rxn mixture was cooled at 0° C. temperature in Ice bath. The 12.0 M aq. HCl (1.27 mL, 15.26 mmol) was added, and reaction mixture was gradually warmed at ambient temperature for 16 h. The LCMS data shows product formation m/z 161.1. The reaction mixture was extracted with ethyl acetate. The combined ethyl acetate layer was separated and dried over anhydrous sodium sulfate. The evaporation of solvent gave crude product. The 1.22 g of product was obtained (Yield Quant.). MS (ESI) mass calcd. for C₅H₈N₂O₄, 160.13; m/z found 161.1 [M+H]⁺.

Step 2: Synthesis of HBS-062-007: Compound HBS-062-005 (1.22 g, 7.63 mmol) was dissolved in anhydrous toluene (10.0 mL). The rxn mixture was cooled at 0° C. temperature in the Ice bath. The anhydrous TFA (1.6 mL, 11.44 mmol) was added, and reaction mixture was gradually warmed at ambient temperature for 16 h. The LCMS data shows product formation m/z 143.1. The reaction mixture was concentrated under reduced pressure to obtain the crude product. The crude product was purified by ISCO combi-flash chromatography system, Mobile phase: EtOAc:Hexane gradient. The 1.02 g of product was obtained (Yield 94.1%). MS (ESI) mass calcd. for C₅H₆N₂O₃, 142.11; m/z found 143.1 [M+H]⁺.

Step 3: Synthesis of HBS-062-009: Compound HBS-062-007 (1.0 g, 7.18 mmol) was dissolved in Xylene (10.0 mL). The ethyl propiolate (0.95 mL, 9.33 mmol) was added, and reaction mixture was heated at 120° C. temperature for 6 h. The LCMS data shows product formation m/z 197.1. The reaction mixture was concentrated under reduced pressure to obtain the crude product. The crude product was purified by ISCO combi-flash chromatography system, Mobile phase: EtOAc:Hexane gradient. The 1.0 g of product was obtained (Yield 71.0%). MS (ESI) mass calcd. for C₉H₁₂N₂O₃, 196.20; m/z found 197.1 [M+H]⁺.

Step 4: Synthesis of HBS-062-010: Compound HBS-062-009 (1.0 g, 5.1 mmol) was dissolved in DCM (15.0 mL). The NBS (1.0 g, 5.61 mmol) was added and rxn mixture was stirred at ambient temperature for 16 h. The LCMS data shows product formation m/z 275.0. The reaction mixture was diluted with water and the product was extracted with DCM. The combined DCM layer was separated and dried over anhydrous sodium sulfate. The evaporation of solvent gave crude product. The crude product was purified by ISCO combi-flash system, Mobile phase: EtOAc:Hexane gradient. The 1.4 g of product was obtained (Yield 99.7%). MS (ESI) mass calcd. for C₉H₁₁BrN₂O₃, 275.1; m/z found 275.0 [M+H]⁺.

Step 5: Synthesis of HBS-052-011: Compound HBS-062-010 (0.4 g, 1.45 mmol) and Phenylboronic acid (0.27 g, 2.18 mmol) were dissolved in mixture of Dioxane/water (9:1 v/v mL). The anhydrous K₂CO₃ (0.6 g, 4.36 mmol) was added followed by Pd(dppf)Cl₂·DCM₂ (0.06 g, 0.073 mmol). The rxn mixture was stirred at 100° C. temperature under N₂ atm. for 6 h. The LCMS data shows product formation m/z 273.1. The rxn mixture was filtered over celite bed and washed with ethyl acetate. The filtrate was concentrated under reduced pressure to obtain the crude product. The crude product was purified by ISCO combi-flash chromatography system, Mobile phase: EtOAc:Hexane gradient. The 0.39 g of product was obtained (Yield 95.5%). MS (ESI) mass calcd. for C₁₅H₁₆N₂O₃, 272.3; m/z found 273.1 [M+H]⁺.

Step 6: Synthesis of HBS-062-013: Compound HBS-062-011 (0.39 g, 1.43 mmol) was dissolved in MeOH (8.0 mL). The 1.0 N aq. solution of NaOH (4.3 mL, 4.3 mmol) was added and rxn mixture was refluxed for 6 h. The LCMS data shows product formation m/z 245.1. The rxn mixture was concentrated under reduced pressure and diluted with water. The aq. layer was acidified with 2.0 M aq. HCl solution (pH=5). The ppts were filtered and washed with water (2.0 mL×3) to obtain 0.23 g of solid product (Yield 65.8%). MS (ESI) mass calcd. for C₁₃H₁₂N₂O₃, 244.25; m/z found 245.1 [M+H]⁺.

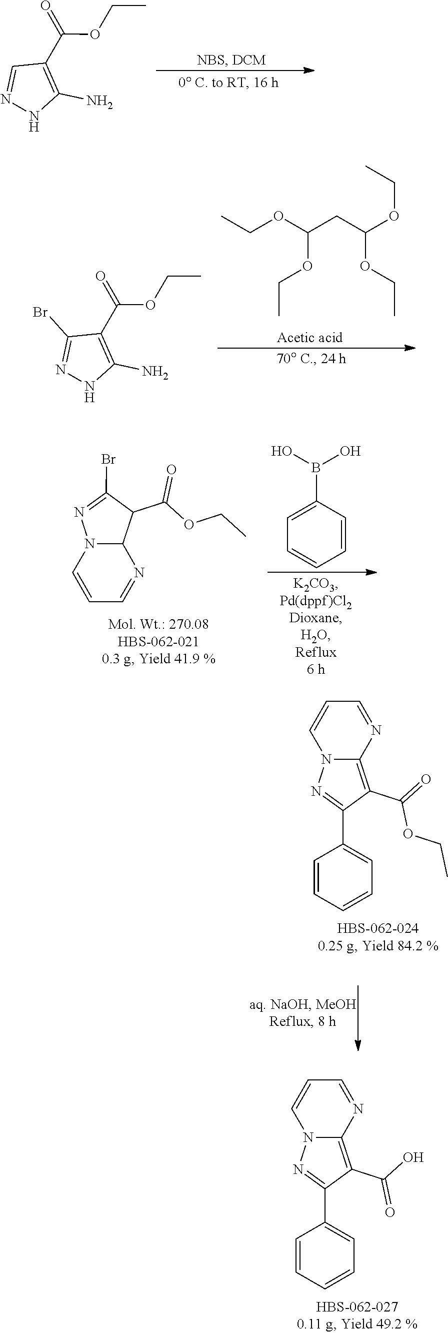

Step 1: Synthesis of HBS-062-019: Ethyl 5-amino-1H-Pyrazole-4-carboxylate (1.0 g, 6.44 mmol) was dissolved in DCM (15.0 mL). The reaction mixture was cooled at 0° C. temperature in Ice bath. The NBS (1.38 g, 7.73 mmol) was added and rxn mixture was stirred at ambient temperature for 16 h. The LCMS data shows product formation m/z 236.0. The reaction mixture was diluted with aq. saturated solution of NaHCO₃. The product was extracted with DCM. The combined DCM layer was separated and dried over anhydrous sodium sulfate. The evaporation of solvent gave crude product. The crude product was purified by ISCO combi-flash system, Mobile phase: DCM:Methanol gradient. The 0.62 g of product was obtained (Yield 41.1%). MS (ESI) mass calcd. for C₆H₈BrN₃O₂, 234.05; m/z found 236.0 [M+H]⁺.

Step 2: Synthesis of HBS-062-021: Compound HBS-062-019 (0.62 g, 2.65 mmol) and 1,1,3,3-Tetraethoxy propane (0.76 mL, 3.18 mmol) were dissolved in anhydrous Acetic acid (10.0 mL). The reaction mixture was heated at 70° C. temperature for 24 h. The LCMS data shows product formation m/z 272.0. The reaction mixture was concentrated under reduced pressure to obtain the crude product. The crude product was diluted with water and neutralized with aq. saturated solution of NaHCO₃. The ppts were filtered and dried to obtain 0.3 g of product (Yield 41.9%). MS (ESI) mass calcd. for C₉H₈BrN₃O₂, 270.08; m/z found 272.0 [M+H]⁺.

Step 3: Synthesis of HBS-062-024: Compound HBS-062-021 (0.3 g, 1.11 mmol) and Phenylboronic acid (0.2 g, 1.67 mmol) were dissolved in mixture of Dioxane/water (7:1 v/v mL). The anhydrous K₂CO₃ (0.46 g, 3.33 mmol) was added followed by Pd(dppf)Cl₂·DCM₂ (0.045 g, 0.056 mmol). The rxn mixture was stirred at 100° C. temperature under N₂ atm. for 4 h. The LCMS data shows product formation m/z 268.1. The rxn mixture was filtered over celite bed and washed with ethyl acetate. The filtrate was concentrated under reduced pressure to obtain the crude product. The crude product was purified by ISCO combi-flash chromatography system, Mobile phase: EtOAc:Hexane gradient. The 0.25 g of product was obtained (Yield 84.2%). MS (ESI) mass calcd. for C₁₅H₁₃N₃O₂, 267.28; m/z found 268.1 [M+H]⁺.

Step 4: Synthesis of HBS-062-027: Compound HBS-062-024 (0.25 g, 0.94 mmol) was dissolved in MeOH (6.0 mL). The 1.0 N aq. solution of NaOH (1.9 mL, 1.87 mmol) was added and rxn mixture was refluxed for 8 h. The LCMS data shows product formation m/z 240.1. The rxn mixture was concentrated under reduced pressure and diluted with water. The aq. layer was acidified with 2.0 M aq. HCl solution (pH=5). The ppts were filtered and washed with water (2.0 mL×3) to obtain 0.11 g of solid product (Yield 49.2%). MS (ESI) mass calcd. for C₁₃H₉N₃O₂, 239.23; m/z found 240.1 [M+H]⁺.

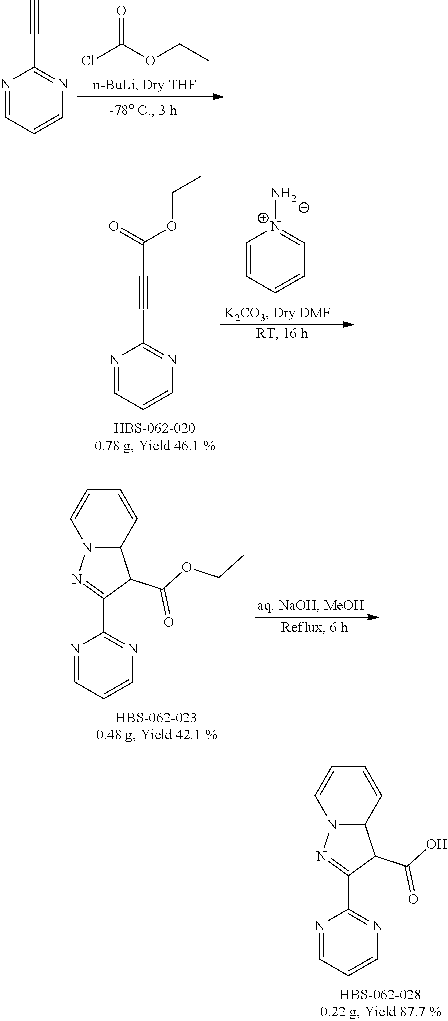

Step 1: Synthesis of HBS-062-020: 1-Ethynyl pyrimidine (1.0 g, 9.61 mmol) was dissolved in anhydrous THF (12.0 mL). The n-BuLi (7.2 mL, 11.53 mmol) was added at −78° C. temperature and rxn mixture was stirred at −78° C. temperature for 30.0 min. The ethyl chloroformate (1.4 mL, 14.41 mmol) was added at −78° C. temperature and rxn mixture was gradually warmed to ambient temperature for 3 h. The LCMS data shows product formation m/z 177.1. The rxn mixture was diluted with aq. NH₄Cl solution and product was extracted with ethyl acetate. The combined ethyl acetate layer was separated and dried over anhydrous sodium sulfate. The evaporation of solvent gave crude product. The crude product was purified by ISCO combi-flash system, Mobile phase: DCM:MeOH gradient. The 0.78 g of product was obtained (Yield 46.1%). MS (ESI) mass calcd. for C₉H₈N₂O₂, 176.17; m/z found 177.1 [M+H]⁺.

Step 2: Synthesis of HBS-062-023: Compound HBS-062-020 (0.75 g, 4.25 mmol) and 1-Aminopyridinium iodide (1.13 g, 5.11 mmol) were dissolved in anhydrous DMF (10.0 mL). The anhydrous K₂CO₃ (1.47 g, 10.63 mmol) was added, and reaction mixture was stirred at ambient temperature for 16 h. The LCMS data shows product formation m/z 269.1. The rxn mixture was diluted with water and the product was extracted with ethyl acetate. The combined ethyl acetate layer was separated and dried over anhydrous sodium sulfate. The evaporation of solvent gave crude product. The crude product was purified by ISCO combi-flash system, Mobile phase: Ethyl acetate:Hexane gradient. The 0.48 g of product was obtained (Yield 42.1%). MS (ESI) mass calcd. for C₁₄H₁₂N₄O₂, 268.27; m/z found 269.1 [M+H]⁺.

Step 3: Synthesis of HBS-062-028: Compound HBS-062-023 (0.28 g, 1.04 mmol) was dissolved in MeOH (8.0 mL). The 1.0 N aq. NaOH solution (2.1 mL, 2.09 mmol) was added, and reaction mixture was refluxed for 6 h. LCMS shows product formation m/z 241.1. The reaction mixture was concentrated under reduced pressure. The solid was dissolved in water and acidified with 2.0 M aq. HCl solution (pH=5). The ppts were filtered and washed with water (2.0 mL×3) to obtain 0.22 g of solid product (Yield 87.5%). MS (ESI) mass calcd. for C₁₂HsN₄O₂, 240.22; m/z found 241.1 [M+H]⁺.

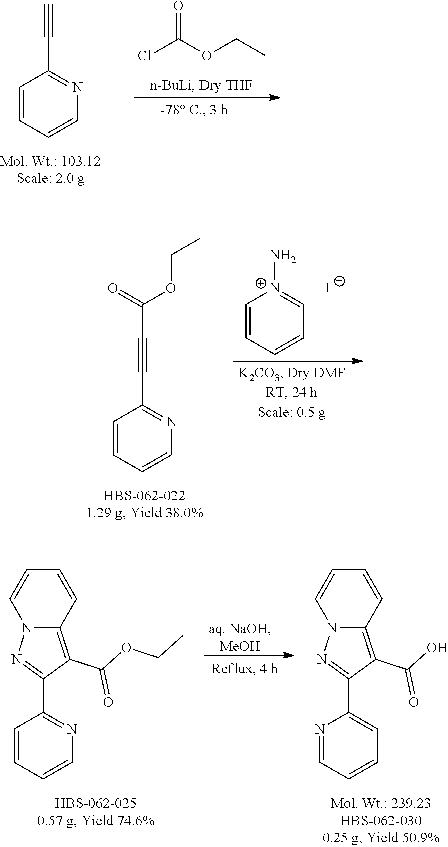

Step 1: Synthesis of HBS-062-022: 1-Ethynyl pyridine (2.0 g, 19.4 mmol) was dissolved in anhydrous THF (15.0 mL). The n-BuLi (14.6 mL, 23.3 mmol) was added at −78° C. temperature and rxn mixture was stirred at −78° C. temperature for 30.0 min. The ethyl chloroformate (2.2 mL, 23.3 mmol) was added at −78° C. temperature and rxn mixture was gradually warmed to ambient temperature for 3 h. The LCMS data shows product formation m/z 176.1. The rxn mixture was diluted with aq. NH₄Cl solution and product was extracted with ethyl acetate. The combined ethyl acetate layer was separated and dried over anhydrous sodium sulfate. The evaporation of solvent gave crude product. The crude product was purified by ISCO combi-flash system, Mobile phase: Ethyl acetate:Hexane gradient. The 1.29 g of product was obtained (Yield 38.0%). MS (ESI) mass calcd. for C₁₀H₉NO₂, 175.18; m/z found 176.1 [M+H]⁺.

Step 2: Synthesis of HBS-062-025: Compound HBS-062-022 (0.5 g, 2.85 mmol) and 1-Aminopyridinium iodide (0.76 g, 3.43 mmol) were dissolved in anhydrous DMF (8.0 mL). The anhydrous K₂CO₃ (0.79 g, 5.71 mmol) was added, and reaction mixture was stirred at ambient temperature for 16 h. The LCMS data shows product formation m/z 268.1. The rxn mixture was diluted with water and the product was extracted with ethyl acetate. The combined ethyl acetate layer was separated and dried over anhydrous sodium sulfate. The evaporation of solvent gave crude product. The crude product was purified by ISCO combi-flash system, Mobile phase: Ethyl acetate:Hexane and DCM:Methanol gradient. The 0.57 g of product was obtained (Yield 74.6%). MS (ESI) mass calcd. for C₁₅H₁₃N₃O₂, 267.28; m/z found 268.1 [M+H]⁺.

Step 3: Synthesis of HBS-062-030: Compound HBS-062-025 (0.56 g, 2.1 mmol) was dissolved in MeOH (8.0 mL). The 1.0 N aq. NaOH solution (4.2 mL, 4.19 mmol) was added, and reaction mixture was refluxed for 4 h. LCMS shows product formation m/z 240.1. The reaction mixture was concentrated under reduced pressure. The solid was dissolved in water and acidified with 2.0 M aq. HCl solution (pH=5). The ppts were filtered and washed with water (2.0 mL×3) to obtain 0.26 g of solid product (Yield 50.9%). MS (ESI) mass calcd. for C₁₃H₉N₃O₂, 239.23; m/z found 240.1 [M+H]⁺.

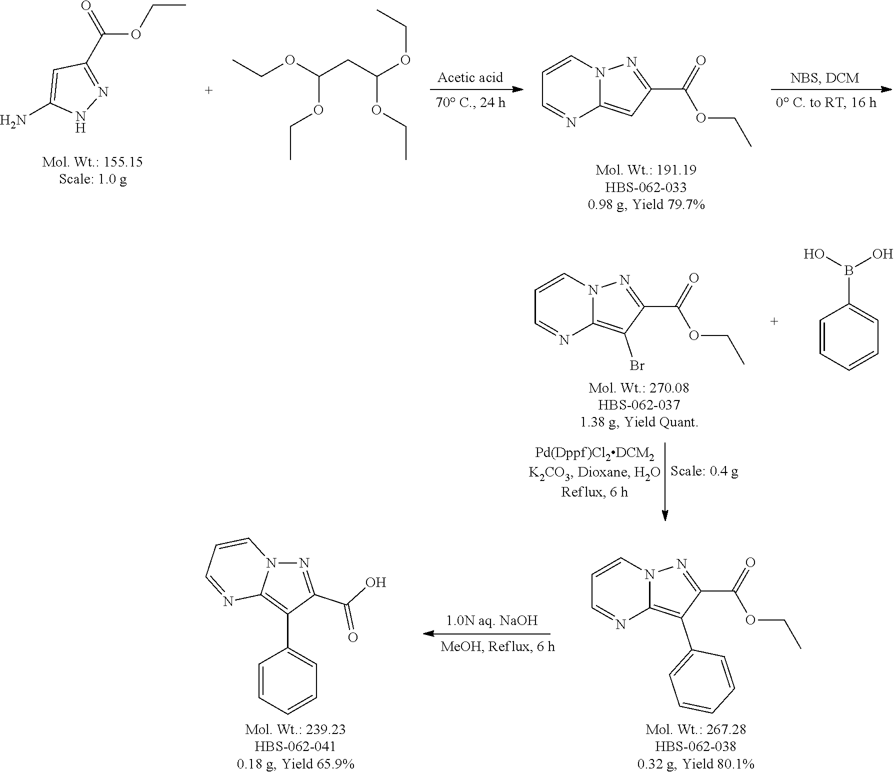

Step 1: Synthesis of HBS-062-033: Ethyl 5-amino-1H-Pyrazole-4-carboxylate (1.0 g, 6.43 mmol) and 1,1,3,3-Tetraethoxy propane (1.85 mL, 7.72 mmol) were dissolved in anhydrous Acetic acid (8.0 mL). The reaction mixture was heated at 70° C. temperature for 24 h. The LCMS data shows product formation m/z 192.1.0. The reaction mixture was concentrated under reduced pressure to obtain the crude product. The crude product was diluted with water and neutralized with aq. saturated solution of NaHCO₃. The product was extracted with DCM. The combined DCM layer was separated and dried over anhydrous sodium sulfate. The evaporation of solvent gave crude product. The crude product was purified by ISCO combi-flash system, Mobile phase: DCM:Methanol gradient. The 0.98 g of product was obtained (Yield 79.7%). MS (ESI) mass calcd. for C₉H₉N₃O₂, 191.19; m/z found 192.1 [M+H]⁺.

Step 2: Synthesis of HBS-062-037: Compound HBS-062-033 (0.98 g, 5.13 mmol) was dissolved in DCM (15.0 mL). The reaction mixture was cooled at 0° C. temperature in Ice bath. The NBS (1.0 g, 5.64 mmol) was added and rxn mixture was stirred at ambient temperature for 16 h. The LCMS data shows product formation m/z 272.0. The reaction mixture was diluted with aq. saturated solution of NaHCO₃. The product was extracted with DCM. The combined DCM layer was separated and dried over anhydrous sodium sulfate. The evaporation of solvent gave 1.38 g of crude product (Yield Quant.). MS (ESI) mass calcd. for C₉H₈BrN₃O₂, 270.08; m/z found 272.0 [M+H]⁺.

Step 3: Synthesis of HBS-062-038: Compound HBS-062-037 (0.4 g, 1.48 mmol) and Phenylboronic acid (0.27 g, 2.22 mmol) were dissolved in mixture of Dioxane/water (8:1 v/v mL).

The anhydrous K₂CO₃ (0.61 g, 4.44 mmol) was added followed by Pd(dppf)Cl₂·DCM₂ (0.06 g, 0.074 mmol). The rxn mixture was stirred at 100° C. temperature under N₂ atm. for 6 h. The LCMS data shows product formation m/z 268.1. The rxn mixture was filtered over celite bed and washed with ethyl acetate. The filtrate was concentrated under reduced pressure to obtain the crude product. The crude product was purified by ISCO combi-flash chromatography system, Mobile phase: EtOAc:Hexane gradient. The 0.32 g of product was obtained (Yield 80.1%). MS (ESI) mass calcd. for C₁₅H₁₃N₃O₂, 267.28; m/z found 268.1 [M+H]⁺.

Step 4: Synthesis of HBS-062-041: Compound HBS-062-038 (0.32 g, 1.19 mmol) was dissolved in MeOH (5.0 mL). The 1.0 N aq. solution of NaOH (2.4 mL, 2.37 mmol) was added and rxn mixture was refluxed for 6 h. The LCMS data shows product formation m/z 240.1. The rxn mixture was concentrated under reduced pressure and diluted with water. The aq. layer was acidified with 2.0 M aq. HCl solution (pH=5). The ppts were filtered and washed with water (2.0 mL×3) to obtain 0.18 g of solid product (Yield 65.9%). MS (ESI) mass calcd. for C₁₃H₉N₃O₂, 239.23; m/z found 240.1 [M+H]⁺.

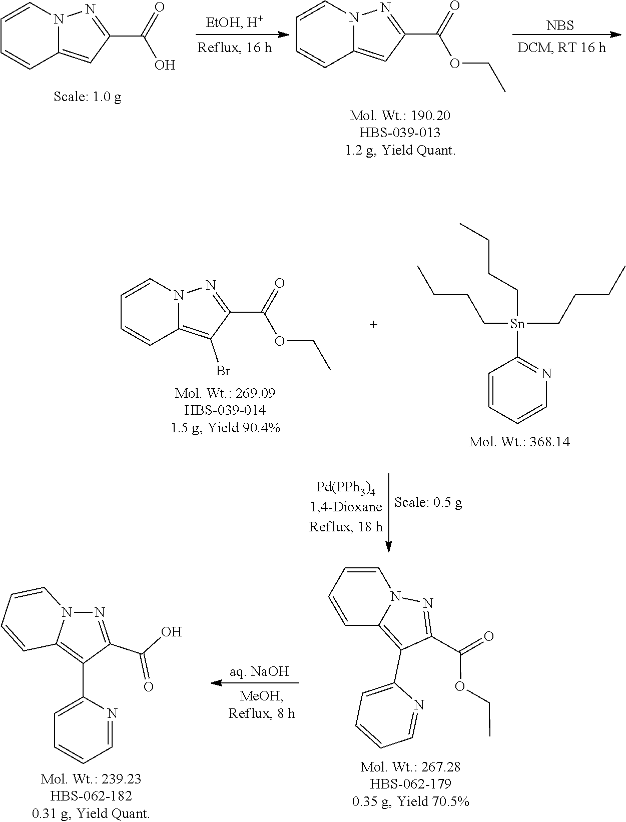

Step-1: Synthesis of HBS-039-013: The pyrazolo[1,5-a]pyridine-2-carboxylic acid (1.0 g, 6.17 mmol) was dissolved in ethanol (20.0 mL). The catalytic amount of conc. sulfuric acid (0.5 mL) was added and rxn mixture was refluxed for 16 h. The LCMS data shows product formation m/z 191.1. The rxn mixture was concentrated under reduced pressure and neutralized with saturate aq. solution of sodium bicarbonate. The product was extracted with ethyl acetate. The ethyl acetate layer was separated and dried over anhydrous Na₂SO₄. The evaporation of solvent gave product. The 1.2 g of product was obtained (Yield Quant.) MS (ESI) mass calcd. for C₁₀H₁₀N₂O₂, 190.2; m/z found 191.1 [M+H]⁺.

Step-2: Synthesis of HBS-039-014: The HBS-039-013 (1.17 g, 6.17 mmol) was dissolved in DCM (25.0 mL). The NBS (1.1 g, 6.17 mmol) was added and rxn mixture was stirred at ambient temperature for 16 h. The LCMS data shows product formation m/z 271.0. The rxn mixture was concentrated under reduced pressure to obtain the crude product. The crude product was purified by ISCO combi-flash chromatography system, Mobile phase: EtOAc:Hexane gradient.

The 1.5 g of product was obtained (Yield 90.4%). MS (ESI) mass calcd. for C₁₀H₉BrN₂O₂, 269.1; m/z found 271.0 [M+H]⁺.

Step 3: Synthesis of HBS-062-179: Compound HBS-039-014 (0.5 g, 1.86 mmol) and 2-(tributyl stannyl)-pyridine (1.36 g, 3.72 mmol) were dissolved in 1,4-Dioxane (10.0 mL). The Pd(PPh₃)₄ (0.214 g, 0.18 mmol) was added, and reaction mixture was heated at 115° C. temperature under N₂ atm. for 18 h. The LCMS data shows product formation m/z 268.0. The rxn mixture was filtered over celite bed and washed with ethyl acetate. The filtrate was concentrated under reduced pressure to obtain the crude product. The crude product was purified by ISCO combi-flash chromatography system, Mobile phase: EtOAc:Hexane gradient.

The 0.35 g of product was obtained (Yield 70.5%). MS (ESI) mass calcd. for C₁₅H₁₃N₃O₂, 267.28; m/z found 268.0 [M+H]⁺.

Step 4: Synthesis of HBS-062-182: Compound HBS-062-179 (0.35 g, 1.31 mmol) was dissolved in MeOH (5.0 mL). The 1.0 N aq. solution of NaOH (2.62 mL, 2.62 mmol) was added and rxn mixture was refluxed for 8 h. The LCMS data shows product formation m/z 240.1. The rxn mixture was concentrated under reduced pressure and diluted with water. The aq. layer was acidified with 2.0 M aq. HCl solution (pH=5). The ppts were filtered and washed with water (2.0 mL×3) to obtain 0.31 g of product (Yield Quant.). MS (ESI) mass calcd. for C₁₃H₉N₃O₂, 239.23; m/z found 240.1 [M+H]⁺.

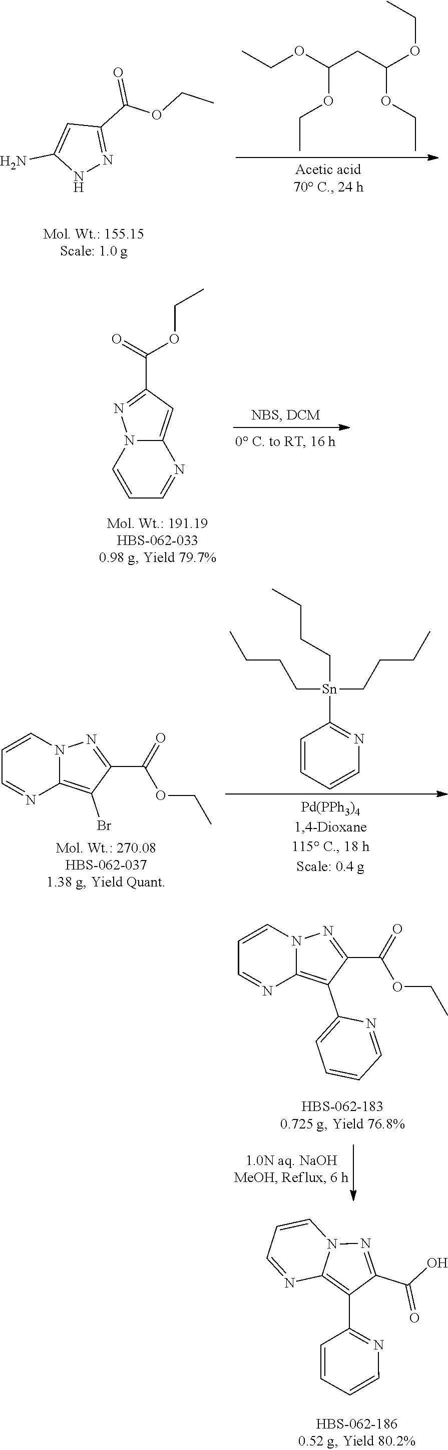

Step 1: Synthesis of HBS-062-033: Ethyl 5-amino-1H-Pyrazole-4-carboxylate (1.0 g, 6.43 mmol) and 1,1,3,3-Tetraethoxy propane (1.85 mL, 7.72 mmol) were dissolved in anhydrous Acetic acid (8.0 mL). The reaction mixture was heated at 70° C. temperature for 24 h. The LCMS data shows product formation m/z 192.1.0. The reaction mixture was concentrated under reduced pressure to obtain the crude product. The crude product was diluted with water and neutralized with aq. saturated solution of NaHCO₃. The product was extracted with DCM. The combined DCM layer was separated and dried over anhydrous sodium sulfate. The evaporation of solvent gave crude product. The crude product was purified by ISCO combi-flash system, Mobile phase: DCM:Methanol gradient. The 0.98 g of product was obtained (Yield 79.7%). MS (ESI) mass calcd. for C₉H₉N₃O₂, 191.19; m/z found 192.1 [M+H]⁺.

Step 2: Synthesis of HBS-062-037: Compound HBS-062-033 (0.98 g, 5.13 mmol) was dissolved in DCM (15.0 mL). The reaction mixture was cooled at 0° C. temperature in Ice bath.

The NBS (1.0 g, 5.64 mmol) was added and rxn mixture was stirred at ambient temperature for 16 h. The LCMS data shows product formation m/z 272.0. The reaction mixture was diluted with aq. saturated solution of NaHCO₃. The product was extracted with DCM. The combined DCM layer was separated and dried over anhydrous sodium sulfate. The evaporation of solvent gave 1.38 g of crude product (Yield Quant.). MS (ESI) mass calcd. for C₉H₈BrN₃O₂, 270.08; m/z found 272.0 [M+H]⁺.

Step 3: Synthesis of HBS-062-183: Compound HBS-062-037 (0.95 g, 3.52 mmol) and 2-(tributyl stannyl)-pyridine (1.94 g, 5.28 mmol) were dissolved in 1,4-Dioxane (12.0 mL). The Pd(PPh₃)₄ (0.41 g, 0.35 mmol) was added, and reaction mixture was heated at 115° C. temperature under N₂ atm. for 18 h. The 0.05 eq Pd(PPh₃)₄ was added to consume the starting material. The LCMS data shows product formation m/z 269.0. The rxn mixture was filtered over celite bed and washed with ethyl acetate. The filtrate was concentrated under reduced pressure to obtain the crude product. The crude product was purified by ISCO combi-flash chromatography system, Mobile phase: EtOAc:Hexane gradient. The 0.725 g of product was obtained (Yield 76.8%). MS (ESI) mass calcd. for C₁₄H₁₂N₄O₂, 268.27; m/z found 269.0 [M+H]⁺.

Step 4: Synthesis of HBS-062-186: Compound HBS-062-183 (0.73 g, 2.7 mmol) was dissolved in MeOH (8.0 mL). The 1.0 N aq. solution of NaOH (5.4 mL, 5.4 mmol) was added and rxn mixture was refluxed for 6 h. The LCMS data shows product formation m/z 241.1. The rxn mixture was concentrated under reduced pressure and diluted with water. The aq. layer was acidified with 2.0 M aq. HCl solution (pH=5). The ppts were filtered and washed with water (2.0 mL×3) to obtain 0.52 g of product (Yield 80.2%). MS (ESI) mass calcd. for C₁₂H₈N₄O₂, 240.22; m/z found 241.1 [M+H]⁺.

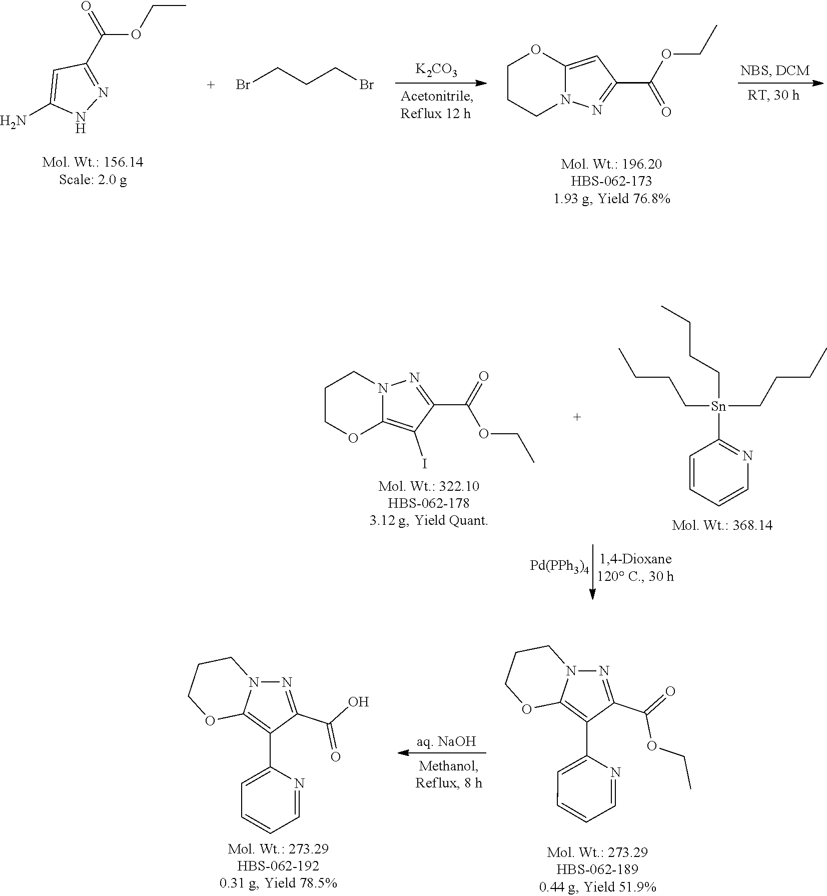

Step 1: Synthesis of HBS-062-173: Ethyl-5-hydroxy-1H-pyrazole-3-carboxylate (2.0 g, 12.81 mmol) was dissolved in anhydrous Acetonitrile (40.0 mL). The anhydrous K₂CO₃ (7.1 g, 51.24 mmol) was added, and reaction mixture was stirred at ambient temperature for 10.0 min. The 1,3-dibromo propane (1.43 mL, 14.1 mmol) was added, and reaction mixture was heated at reflux for 12 h. The LCMS data shows product formation m/z 197.1. The reaction mixture cooled at ambient temperature and filtered over celite. The filtrate was concentrated under reduced pressure to obtain the crude product. The crude product was purified by ISCO combi-flash chromatography system, Mobile phase: EtOAc:Hexane gradient. The 1.93 g of product was obtained (Yield 76.8%). MS (ESI) mass calcd. for C₉H₁₂N₂O₃, 196.20; m/z found 197.1 [M+H]⁺.

Step 2: Synthesis of HBS-062-178: Compound HBS-062-173 (1.9 g, 9.68 mmol) was dissolved in DCM (30.0 mL). The NIS (2.61 g, 11.62 mmol) was added and rxn mixture was stirred at ambient temperature for 30 h. The 0.6 eq of NIS was added to consume the starting material. The LCMS data shows product formation m/z 323.0. The reaction mixture was diluted with water and the product was extracted with DCM. The DCM layer was separated and washed with aq. sodium thiosulphate solution. The combined DCM layer was separated and dried over anhydrous sodium sulfate. The evaporation of solvent gave crude product. The crude product was purified by ISCO combi-flash system, Mobile phase: EtOAc:Hexane gradient. The 3.12 g of product was obtained (Yield Quant.). MS (ESI) mass calcd. for C₉H₁₁N₂O₃, 322.1; m/z found 323.0 [M+H]⁺.

Step 3: Synthesis of HBS-062-189: Compound HBS-062-178 (1.0 g, 3.11 mmol) and 2-(tributyl stannyl)-pyridine (1.14 g, 3.11 mmol) were dissolved in 1,4-Dioxane (12.0 mL). The Pd(PPh₃)₄ (0.36 g, 0.31 mmol) was added, and reaction mixture was heated at 120° C. temperature under N₂ atm. for 30 h. The LCMS data shows product formation m/z 274.0. The rxn mixture was filtered over celite bed and washed with ethyl acetate. The filtrate was concentrated under reduced pressure to obtain the crude product. The crude product was purified by ISCO combi-flash chromatography system, Mobile phase: EtOAc:Hexane gradient and EtOAc:Methanol (95:05, v/v mL) gradient. The 0.44 g of product was obtained (Yield 51.9%). MS (ESI) mass calcd. for C₁₄H₁₅N₃O₃, 273.3; m/z found 274.0 [M+H]⁺.

Step 4: Synthesis of HBS-062-192: Compound HBS-062-189 (0.44 g, 1.61 mmol) was dissolved in MeOH (8.0 mL). The 1.0 N aq. solution of NaOH (3.22 mL, 3.22 mmol) was added and rxn mixture was refluxed for 8 h. The LCMS data shows product formation m/z 246.1. The rxn mixture was concentrated under reduced pressure and diluted with water. The aq. layer was acidified with 2.0 M aq. HCl solution (pH=5). The ppts were filtered and washed with water (2.0 mL×3) to obtain 0.31 g of product (Yield 78.5%). MS (ESI) mass calcd. for C₁₂H₁₁N₃O₃, 245.23; m/z found 246.1 [M+H]⁺.

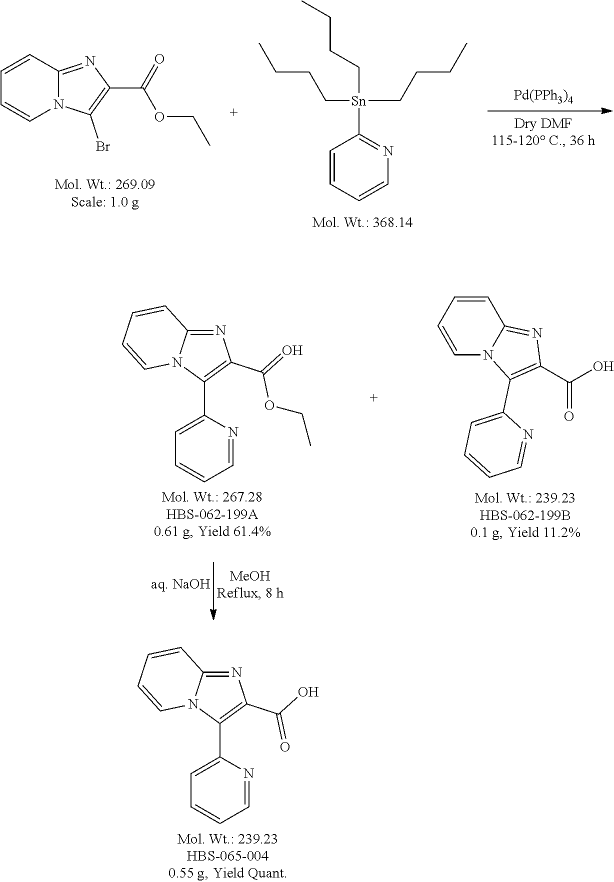 [SMILES: CCOC(=O)c1nc2ccccn2c1Br ; CCC[CH2][Sn]([CH2]CCC)([CH2]CCC)[c]1ccccn1 ; CCOC(=O)c1nc2ccccn2c1-c1ccccn1 ; O=C(O)c1nc2ccccn2c1-c1ccccn1 ; O=C(O)c1nc2ccccn2c1-c1ccccn1 ; "Pd(PPh3)4"]

Step 1: Synthesis of HBS-062-199: 3-Bromoimidazo[1,2-a]pyridine-2-carboxylic acid ethyl ester (1.0 g, 3.72 mmol) and 2-(tributyl stannyl)-pyridine (1.5 g, 4.1 mmol) were dissolved in Dry DMF (12.0 mL). The Pd(PPh₃)₄ (0.43 g, 0.37 mmol) was added, and reaction mixture was heated at 115-120° C. temperature under N₂ atm. for 36 h. The 0.05 eq of Pd(PPh₃)₄ was added to consume the starting material during the reaction. The LCMS data shows product formation m/z 268.1 and acid side product m/z 240.1. The reaction mixture was diluted with water and the product was extracted with DCM. The DCM layer was separated and washed with aq. sodium thiosulphate solution. The combined DCM layer was separated and dried over anhydrous sodium sulfate. The evaporation of solvent gave crude product. The crude product was purified by ISCO combi-flash chromatography system, Mobile phase: EtOAc:Hexane gradient and EtOAc:Methanol (95:05, v/v mL) gradient. The 0.61 g of product was obtained (Yield 61.4%). MS (ESI) mass calcd. for C₁₅H₁₃N₃O₂, 267.28; m/z found 268.1 [M+H]⁺ and 0.1 g of acid side product was obtained (Yield 11.2%). MS (ESI) mass calcd. for C₁₃H₉N₃O₂, 239.23; m/z found 240.1 [M+H]⁺.

Step 2: Synthesis of HBS-065-004: Compound HBS-062-199A (0.61 g, 2.28 mmol) was dissolved in MeOH (8.0 mL). The 1.0 N aq. solution of NaOH (4.6 mL, 4.56 mmol) was added and reaction mixture was refluxed for 8 h. The LCMS data shows product formation m/z 240.9. The rxn mixture was concentrated under reduced pressure and diluted with water. The aq. layer was acidified with 2.0 M aq. HCl solution (pH=5). The aq. layer was concentrated to obtain 0.55 g of product (Yield Quant.). MS (ESI) mass calcd. for C₁₃H₉N₃O₂, 239.23; m/z found 240.9 [M+H]⁺.

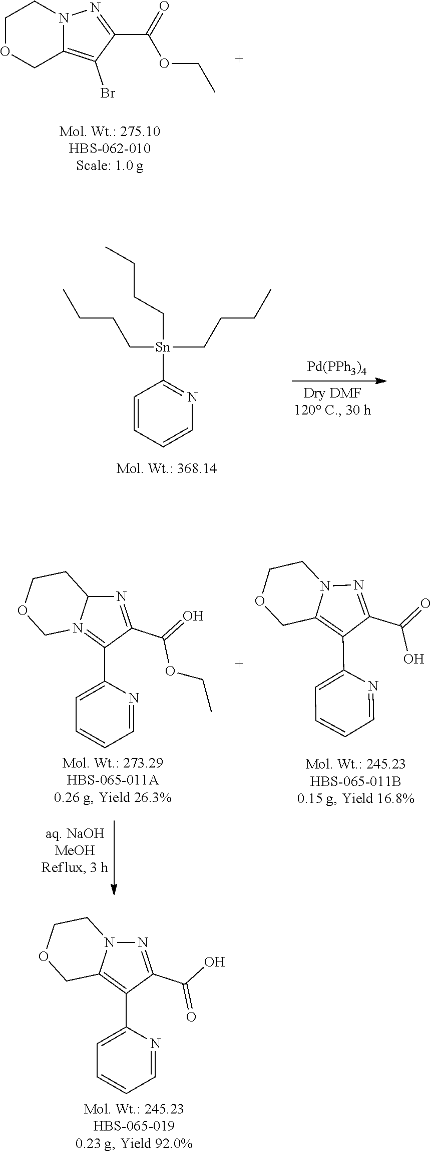

Step 1: Synthesis of HBS-065-011: HBS-062-010 (1.0 g, 3.64 mmol) and 2-(tributyl stannyl)-pyridine (1.6 g, 4.36 mmol) were dissolved in Dry DMF (12.0 mL). The Pd(PPh₃)₄ (0.42 g, 0.36 mmol) was added, and reaction mixture was heated at 120° C. temperature under N₂ atm. for 30 h. The 0.05 eq of Pd(PPh₃)₄ was added to consume the starting material during the reaction. The LCMS data shows product formation m/z 274.0 and acid side product m/z 246.0. The reaction mixture was diluted with water and the product was extracted with DCM. The DCM layer was separated and washed with aq. sodium thiosulphate solution. The combined DCM layer was separated and dried over anhydrous sodium sulfate. The evaporation of solvent gave crude product. The crude product was purified by ISCO combi-flash chromatography system, Mobile phase: EtOAc:Hexane gradient and DCM:Methanol gradient. The 0.26 g of product was obtained (Yield 26.3%). MS (ESI) mass calcd. for C₁₄H₁₅N₃O₃, 273.29; m/z found 274.0 [M+H]⁺ and 0.15 g of acid side product was obtained (Yield 16.8%). MS (ESI) mass calcd. for C₁₂H₁₁N₃O₃, 245.23; m/z found 246.0 [M+H]⁺.

Step 2: Synthesis of HBS-065-019: Compound HBS-065-011A (0.26 g, 0.96 mmol) was dissolved in MeOH (5.0 mL). The 1.0 N aq. solution of NaOH (1.9 mL, 1.91 mmol) was added and reaction mixture was refluxed for 3 h. The LCMS data shows product formation m/z 246.1. The rxn mixture was concentrated under reduced pressure and diluted with water. The aq. layer was acidified with 2.0 M aq. HCl solution (pH=5). The ppts were filtered and aq. layer was concentrated to obtain 0.23 g of total product (Yield 92.0%). MS (ESI) mass calcd. for C₁₂H₁₁N₃O₃, 245.23; m/z found 246.1 [M+H]⁺.

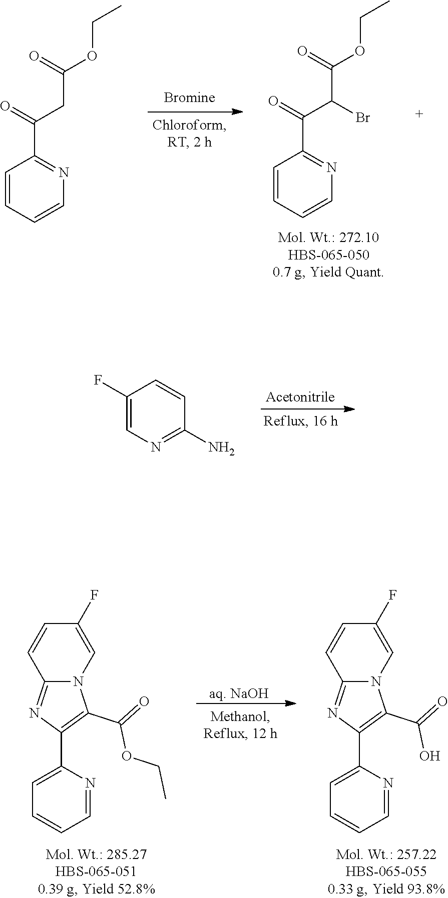 [SMILES: CCOC(=O)CC(=O)c1ccccn1 ; CCOC(=O)C(Br)C(=O)c1ccccn1 ; Nc1ccc(F)cn1 ; CCOC(=O)c1c(-c2ccccn2)nc2ccc(F)cn12 ; O=C(O)c1c(-c2ccccn2)nc2ccc(F)cn12]

Step 1: Synthesis of HBS-065-050: Ethyl picolinoylacetate (0.5 g, 2.59 mmol) was dissolved in Chloroform (12.0 mL). The Bromine (0.13 mL, 2.59 mmol) solution in Chloroform (1.0 mL) was added and reaction mixture was stirred at ambient temperature for 2 h. LCMS shows product formation m/z 274.0. The reaction mixture was diluted with aq. saturated solution of NaHCO₃ and the product was extracted with Chloroform. The Chloroform layer was separated and dried over anhydrous Na₂SO₄. The evaporation of solvent gave 0.7 g of crude product (Yield Quant.). MS (ESI) mass calcd. for C₁₀H₁₀BrNO₃, 272.1; m/z found 274.0 [M+H]⁺.

Step 2: Synthesis of HBS-065-051: Compound HBS-065-050 (0.7 g, 2.59 mmol) was dissolved in anhydrous Acetonitrile (8.0 mL). The 2-amino-5-fluoropyridine (0.29 g, 2.59 mmol) was added and reaction mixture was heated at reflux for 16 h. LCMS shows product formation m/z 286.1. The reaction mixture was diluted with aq. saturated solution of NaHCO₃ and the product was extracted with Ethyl acetate. The combined Ethyl acetate layer was separated and dried over anhydrous sodium sulfate. The evaporation of solvent gave crude product. The crude product was purified by combi-flash system, Mobile phase: DCM:Methanol gradient. The 0.39 g of product was obtained (Yield 52.8%). MS (ESI) mass calcd. for C₁₅H₁₂FN₃O₂, 285.27; m/z found 286.1 [M+H]⁺.

Step 3: Synthesis of HBS-065-055: Compound HBS-065-051 (0.39 g, 1.37 mmol) was dissolved in MeOH (8.0 mL). The 1.0 N aq. NaOH solution (2.7 mL, 2.73 mmol) was added, and reaction mixture was heated at reflux for 12 h. LCMS shows product formation m/z 258.2. The reaction mixture was concentrated under reduced pressure. The solid was dissolved in water and acidified with 2.0 M aq. HCl solution (pH=5). The ppts were filtered and washed with water (2.0 mL×3) to obtain 0.33 g of solid product (Yield 93.8%). MS (ESI) mass calcd. for C₁₃H₈FN₃O₂, 257.22; m/z found 258.2 [M+H]⁺.

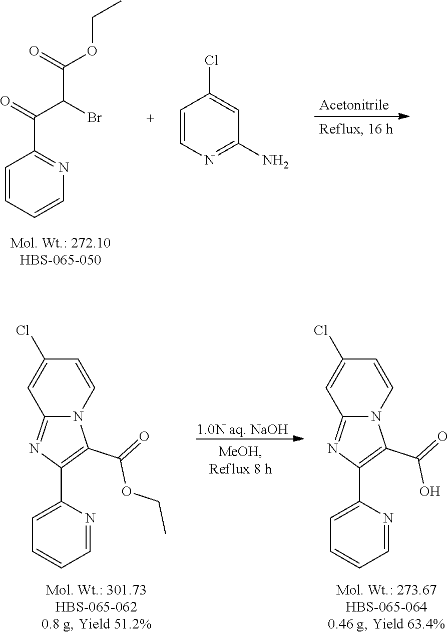 [SMILES: CCOC(=O)C(Br)C(=O)c1ccccn1 ; Nc1cc(Cl)ccn1 ; CCOC(=O)c1c(-c2ccccn2)nc2cc(Cl)ccn12 ; O=C(O)c1c(-c2ccccn2)nc2cc(Cl)ccn12]

Step 1: Synthesis of HBS-065-062: Compound HBS-065-050 (1.41 g, 5.18 mmol) was dissolved in anhydrous Acetonitrile (10.0 mL). The 2-amino-4-chloropyridine (0.67 g, 5.18 mmol) was added and reaction mixture was heated at reflux for 16 h. LCMS shows product formation m/z 302.0. The reaction mixture was diluted with aq. saturated solution of NaHCO₃ and the product was extracted with Ethyl acetate. The combined Ethyl acetate layer was separated and dried over anhydrous sodium sulfate. The evaporation of solvent gave crude product. The crude product was purified by combi-flash system, Mobile phase: DCM:Methanol gradient. The 0.8 g of product was obtained (Yield 51.2%). MS (ESI) mass calcd. for C₁₅H₁₂ClN₃O₂, 301.73; m/z found 302.0 [M+H]⁺.

Step 2: Synthesis of HBS-065-064: Compound HBS-065-062 (0.8 g, 2.65 mmol) was dissolved in MeOH (8.0 mL). The 1.0 N aq. NaOH solution (5.3 mL, 5.30 mmol) was added, and reaction mixture was heated at reflux for 8 h. LCMS shows product formation m/z 274.0. The reaction mixture was concentrated under reduced pressure. The crude was dissolved in water and acidified with 2.0 M aq. HCl solution (pH=5). The ppts were filtered and washed with water (3.0 mL×3) to obtain 0.46 g of solid product (Yield 63.4%). MS (ESI) mass calcd. for C₁₃H₈ClN₃O₂, 273.67; m/z found 274.0 [M+H]⁺.

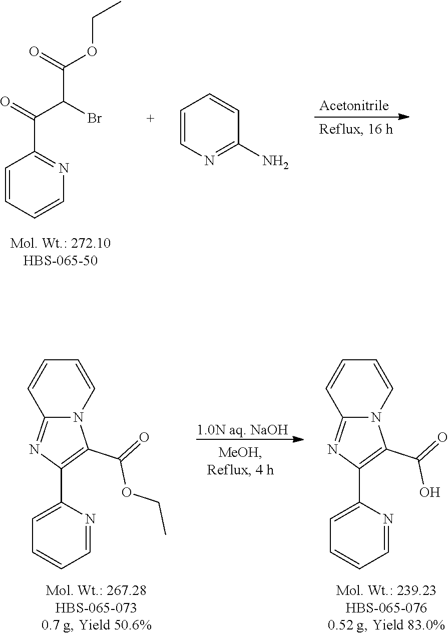 [SMILES: CCOC(=O)C(Br)C(=O)c1ccccn1 ; Nc1ccccn1 ; CCOC(=O)c1c(-c2ccccn2)nc2ccccn12 ; O=C(O)c1c(-c2ccccn2)nc2ccccn12]

Step 1: Synthesis of HBS-065-073: Compound HBS-065-050 (1.41 g, 5.18 mmol) was dissolved in anhydrous Acetonitrile (12.0 mL). The 2-aminopyridine (0.49 g, 5.18 mmol) was added and the reaction mixture was heated at reflux for 16 h. LCMS shows product formation m/z 268.0. The reaction mixture was diluted with aq. saturated solution of NaHCO₃ and the product was extracted with Ethyl acetate. The combined Ethyl acetate layer was separated and dried over anhydrous sodium sulfate. The evaporation of solvent gave crude product. The crude product was purified by combi-flash system, Mobile phase: DCM:Methanol gradient. The 0.7 g of product was obtained (Yield 50.6%). MS (ESI) mass calcd. for C₁₅H₁₃N₃O₂, 267.28; m/z found 268.0 [M+H]⁺.

Step 2: Synthesis of HBS-065-076: Compound HBS-065-073 (0.7 g, 2.62 mmol) was dissolved in MeOH (6.0 mL). The 1.0 N aq. NaOH solution (5.2 mL, 5.24 mmol) was added, and reaction mixture was heated at reflux for 4 h. LCMS shows product formation m/z 240.0. The reaction mixture was concentrated under reduced pressure. The crude was dissolved in water and acidified with 2.0 M aq. HCl solution (pH=5). The ppts were filtered and washed with water (3.0 mL×3) to obtain 0.52 g of solid product (Yield 83.0%). MS (ESI) mass calcd. for C₁₃H₉N₃O₂, 239.23; m/z found 240.0 [M+H]⁺.

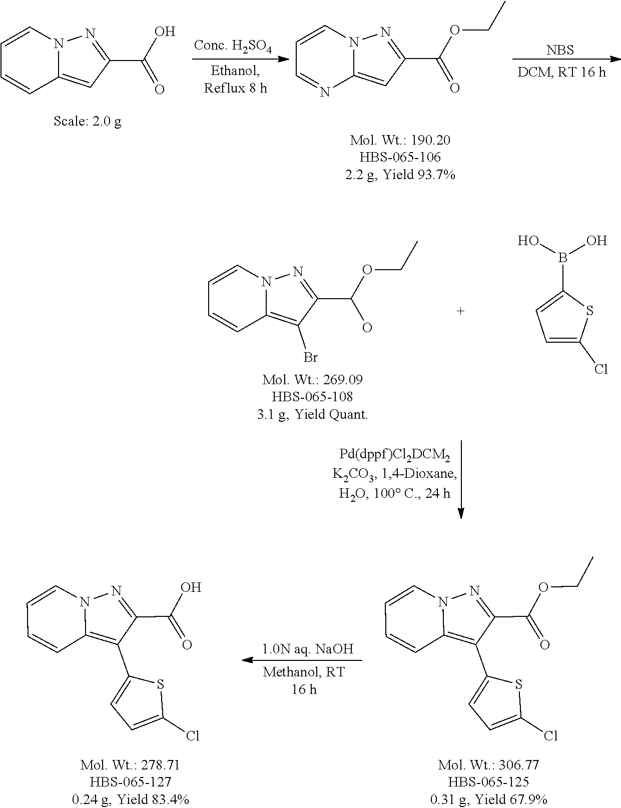

Step-1: Synthesis of HBS-065-106: The pyrazolo[1,5-a]pyridine-2-carboxylic acid (2.0 g, 12.34 mmol) was dissolved in Ethanol (40.0 mL). The catalytic amount of conc. sulfuric acid (0.1 mL) was added and rxn mixture was refluxed for 8 h. The LCMS data shows product formation m/z 191.1. The rxn mixture was concentrated under reduced pressure and neutralized with saturate aq. solution of sodium bicarbonate. The product was extracted with ethyl acetate. The ethyl acetate layer was separated and dried over anhydrous Na₂SO₄. The evaporation of solvent gave product. The 2.2 g of product was obtained (Yield 93.7%) MS (ESI) mass calcd. for C₁₀H₁₀N₂O₂, 190.2; m/z found 191.1 [M+H]⁺.

Step 2: Synthesis of HBS-065-108: Compound HBS-065-106 (2.2 g, 11.57 mmol) was dissolved in DCM (40.0 mL). The NBS (2.27 g, 12.73 mmol) was added and rxn mixture was stirred at ambient temperature for 16 h. The LCMS data shows product formation m/z 270.9.

The rxn mixture was diluted with aq. saturated solution of sodium bicarbonate. The product was extracted with DCM. The combined DCM layer was separated and dried over anhydrous Na₂SO₄. The evaporation of solvent gave product. The crude product was purified by normal phase chromatography system, Mobile phase: EtOAc:Hexane gradient. The 3.11 g of product was obtained (Yield Quant.). MS (ESI) mass calcd. for C₁₀H₉BrN₂O₂, 269.1; m/z found 270.9 [M+H]⁺.

Step 3: Synthesis of HBS-065-125: Compound HBS-065-108 (0.4 g, 1.49 mmol) and 5-Chlorothiophene-2-boronic acid (0.48 g, 2.97 mmol) were dissolved in mixture of Dioxane/water (12:1 v/v mL). The anhydrous K₂CO₃ (0.41 g, 2.97 mmol) was added followed by Pd(dppf)Cl₂·DCM₂ (0.06 g, 0.074 mmol). The rxn mixture was stirred at 100° C. temperature under N₂ atm. for 24 h. The 5-Chlorothiophene-2-boronic acid and catalyst were added to consume the starting material during the reaction. The LCMS data shows product formation m/z 307.0. The rxn mixture was filtered over celite bed and washed with ethyl acetate. The filtrate was concentrated under reduced pressure to obtain the crude product. The crude product was purified by ISCO combi-flash chromatography system, Mobile phase: EtOAc:Hexane gradient. The 0.31 g of product was obtained (Yield 67.9%). MS (ESI) mass calcd. for C₁₄H₁₁ClN₂O₂S, 306.77; m/z found 307.0 [M+H]⁺.

Step 4: Synthesis of HBS-065-127: Compound HBS-065-125 (0.31 g, 1.01 mmol) was dissolved in MeOH (5.0 mL). The 1.0 N aq. solution of NaOH (2.0 mL, 2.0 mmol) was added and reaction mixture was stirred at ambient temperature for 16 h. The LCMS data shows product formation m/z 278.9. The rxn mixture was concentrated under reduced pressure and diluted with water. The aq. layer was acidified with 2.0 M aq. HCl solution (pH=5). The ppts were filtered and washed with water (2.0 mL×3) to obtain 0.24 g of solid product (Yield 83.4%). MS (ESI) mass calcd. for C₁₂H₇ClN₂O₂S, 278.71; m/z found 278.9 [M+H]⁺.

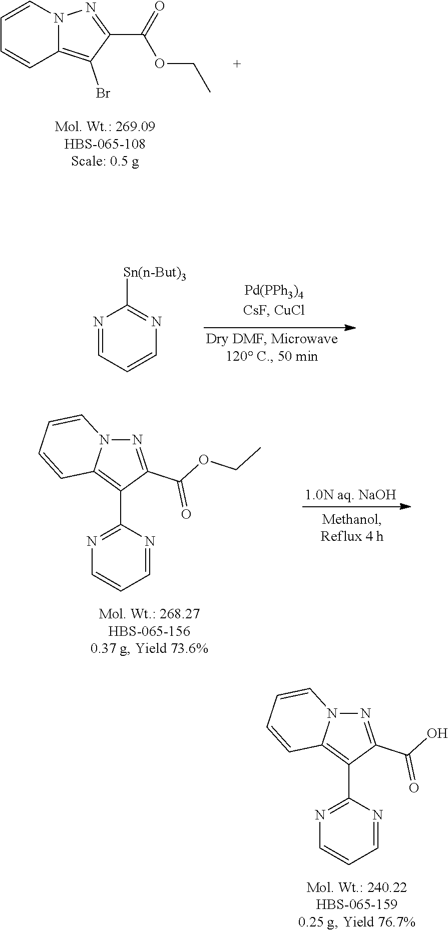

Step 1: Synthesis of HBS-065-156: Compound HBS-065-108 (0.5 g, 1.86 mmol) and 2-(tributylstannyl)-pyrimidine (0.69 g, 1.86 mmol) were dissolved in anhydrous DMF (6.0 mL). The anhydrous CsF (0.85 g, 5.57 mmol) and CuCl (0.024 g, 0.24 mmol) were added followed by Pd(PPh₃)₄ (0.11 g, 0.093 mmol). The rxn mixture was irradiated with microwave radiation at 120° C. temperature for 50.0 min in microwave reactor. The LCMS data shows product formation m/z 269.2. The rxn mixture was filtered over celite bed and washed with ethyl acetate. The filtrate was diluted with water and extracted with Ethyl acetate. The combined Ethyl acetate layer was separated and dried over anhydrous Na₂SO₄. The evaporation of solvent gave crude product. The crude product was purified by ISCO combi-flash chromatography system, Mobile phase: EtOAc:Hexane gradient. The 0.37 g of product was obtained (Yield 73.6%). MS (ESI) mass calcd. for C₁₄H₁₂N₄O₂, 268.27; m/z found 269.2 [M+H]⁺.

Step 2: Synthesis of HBS-065-159: Compound HBS-065-156 (0.37 g, 1.37 mmol) was dissolved in MeOH (8.0 mL). The 1.0 N aq. solution of NaOH (2.74 mL, 2.74 mmol) was added and reaction mixture was refluxed for 4 h. The LCMS data shows product formation m/z 241.1. The rxn mixture was concentrated under reduced pressure and diluted with water. The aq. layer was acidified with 2.0 M aq. HCl solution (pH=5). The ppts were filtered and washed with water (2.0 mL×3) to obtain 0.25 g of solid product (Yield 76.7%). MS (ESI) mass calcd. for C₁₂H₈N₄O₂, 240.22; m/z found 241.1 [M+H]⁺.

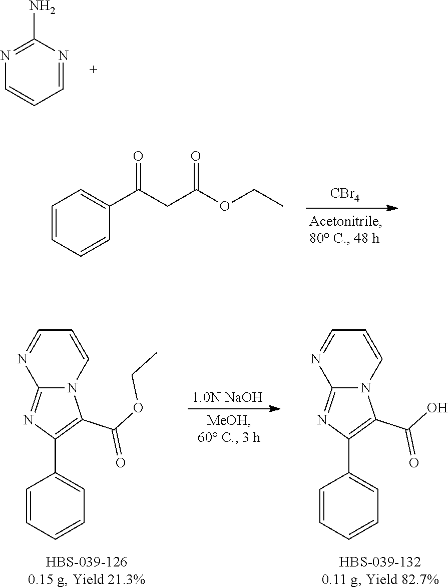 [SMILES: Nc1ncccn1 ; CCOC(=O)CC(=O)c1ccccc1 ; "CBr4" ; CCOC(=O)c1c(-c2ccccc2)nc2ncccn12 ; O=C(O)c1c(-c2ccccc2)nc2ncccn12]

Step 1: Synthesis of HBS-039-126: Ethyl benzoylacetate (0.5 g, 2.6 mmol) was dissolved in anhydrous acetonitrile (10.0 mL). The 2-amino-pyrimidine (0.55 g, 5.76 mmol) was added followed by CBr₄ (1.27 g, 3.84 mmol) and reaction mixture was stirred at 80° C. for 48 h. The reagents were added to consume the starting material. LCMS shows product formation m/z 268.1. The reaction mixture was concentrated under reduced pressure to yield crude product. The crude product was purified by combi-flash system, Mobile phase: EtOAc:Hexane gradient. The 0.15 g of product was obtained (Yield 21.3%). MS (ESI) mass calcd. for C₁₅H₁₃N₃O₂, 267.28; m/z found 268.1 [M+H]⁺.

Step 2: Synthesis of HBS-039-132: Compound HBS-039-126 (0.15 g, 0.55 mmol) was dissolved in MeOH (2.5 mL). The 1.0 N aq. NaOH solution (2.77 mL, 2.76 mmol) was added, and reaction mixture was refluxed for 3 h. LCMS shows product formation m/z 240.1. The reaction mixture was concentrated under reduced pressure. The solid was dissolved in water and acidified with 2.0 M aq. HCl solution (pH=5). The ppts were filtered and washed with water (2.0 mL×3) to obtain 0.11 g of solid product (Yield 82.7%). MS (ESI) mass calcd. for C₁₃H₉N₃O₂, 239.23; m/z found 240.1 [M+H]⁺.

### Synthesis of Secondary Amine Containing Intermediates—Amine Group

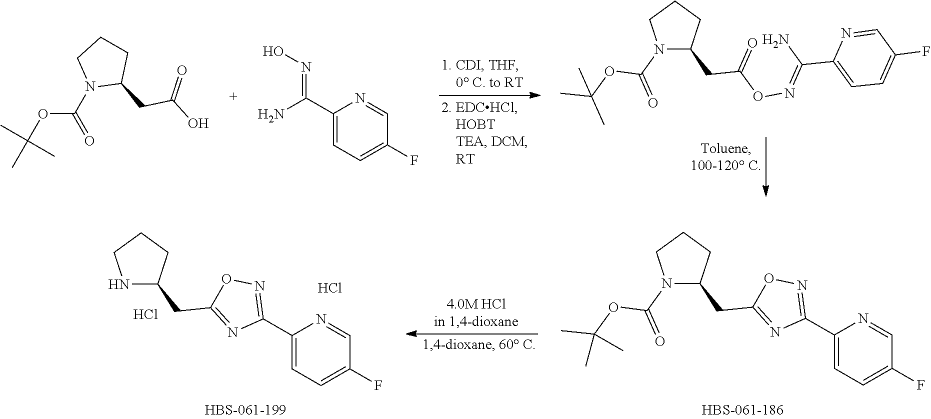

Step 1: Synthesis of HBS-061-186: The carboxylic acid (1.540 g, 6.72 mmol) was dissolved in THF (30 mL). The solution was cooled in an ice bath for 1 h. CDI (1.09 g, 6.72 mmol) was added and after 30 min the ice bath was removed, and the reaction was stirred at RT for 18 h. The hydroxyl amidine (0.694 g, 4.48 mmol) was added and stirring at RT continued. After 24 h observed only 36% conversion to the acyl intermediate by LCMS. Introduced sequentially HOBT (0.908 g, 6.72 mmol), EDC (1.09 g, 6.72 mmol), and TEA (3.11 mL, 22.3 mmol), and CH₂C₁₂ (30 mL) and stirred at RT. LCMS showed conversion to the acyl intermediate was complete after 22 h with m/z 367. The acyl intermediate was isolated by extractive workup between EtOAc and water affording a viscous yellow oil (2.40 g). The oil was dissolved in DCM (15 mL), toluene (25 mL) was added, and the reaction was heated in an open flask to 100-120° C. LCMS showed the product with m/z 349. The reaction mix was dissolved in aq. sat. NaHCO₃ (aq). and extracted with EtOAc. The EtOAc layer was dried over Na₂SO₄, filtered, and evaporated. The crude product was purified by column chromatography, mobile phase: Hexane:EtOAc gradient. Obtained 1.14 g of white solid (Yield=73.3%). MS (ESI) mass calcd. for C₁₇H₂₁FN₄O₃, 348.2; m/z found 349.1 [M+H]⁺.

Step 2: Synthesis of HBS-061-199: Compound HBS-061-186 (1.142 g, 3.28 mmol) was dissolved in anhydrous dioxane (20 mL). The 4.0 M HCl in dioxane (4.1 mL, 16.4 mmol) was added and the reaction was stirred vigorously at 60° C. for 42 h forming a suspension. LCMS showed complete conversion to product m/z 249. The reaction mixture was cooled to RT, filtered, and washed with hexanes to obtain a paste that adhered to the filter paper. The sample was dissolved in MeOH, concentrated, and dried in a vacuum oven to afford 0.807 g of powdery off white solid (Yield=76.7%). MS (ESI) mass calcd. for C₁₂H₁₃FN₄O, 248.1; m/z found 249.1 [M+H]⁺.

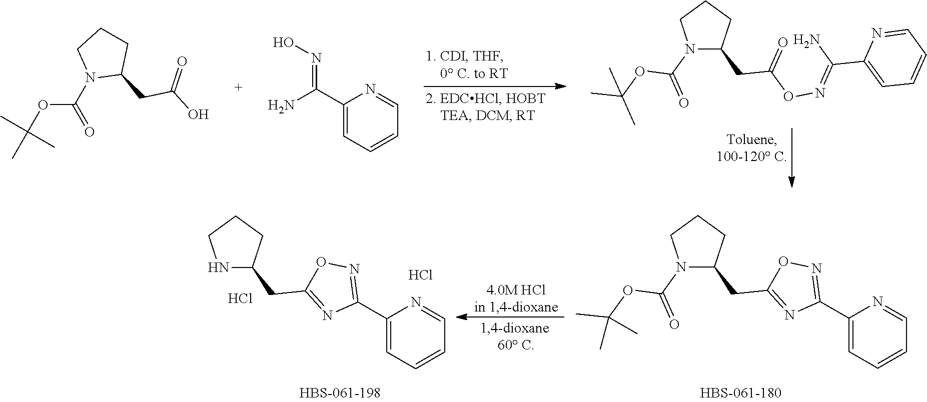

Step 1: Synthesis of HBS-061-180: The carboxylic acid (1.993 g, 8.69 mmol) was dissolved in THF (40 mL). The solution was cooled in an ice bath for 1 h. CDI (1.42 g, 8.76 mmol) was added and after 15 min the ice bath was removed, and the reaction was stirred at RT for 6 h. The hydroxyl amidine (0.795 g, 5.80 mmol) was added and stirring at RT continued. After 72 h observed only 50% conversion to the acyl intermediate by LCMS. Introduced sequentially HOBT (1.17 g, 8.66 mmol), EDC (1.67 g, 8.71 mmol), and TEA (4.0 mL, 28.7 mmol), and CH₂Cl₂ (40 mL) and stirred at RT. LCMS showed conversion to the acyl intermediate was complete after 22 h with m/z 349. The acyl intermediate was isolated by extractive workup between EtOAc and water affording a light-yellow oil (2.87 g). The oil was dissolved in CH₂Cl₂ (25 mL), toluene (30 mL) was added, and the reaction was heated in an open flask to 100° C. LCMS showed the product formation m/z 331. The reaction mix was dissolved in aq. sat. NaHCO₃ and extracted with EtOAc. The EtOAc layer was dried over Na₂SO₄, filtered, and evaporated. The crude product was purified by column chromatography, mobile phase: Hexanes:EtOAc gradient. Obtained 1.65 g of colorless oil (Yield=85.9%). MS (ESI) mass calcd. for C₁₇H₂₂N₄O₃, 330.2; m/z found 331.1 [M+H]⁺.

Step 2: Synthesis of HBS061-198: Compound HBS-061-180 (1.645 g, 4.98 mmol) was dissolved in anhydrous dioxane (30 mL). The 4.0 M HCl in dioxane (6.2 mL, 24.8 mmol) was added and the reaction was stirred vigorously at 60° C. for 42 h. LCMS showed complete conversion to product m/z 231. The reaction mixture was cooled to RT, filtered, and washed with hexanes to obtain a paste that adhered to the filter paper. The sample was dissolved in MeOH, concentrated, and dried in a vacuum oven to afford 1.47 g of crusty beige solid (Yield=97.1%). MS (ESI) mass calcd. for C₁₂H₁₄N₄O, 230.1; m/z found 231.1 [M+H]⁺.

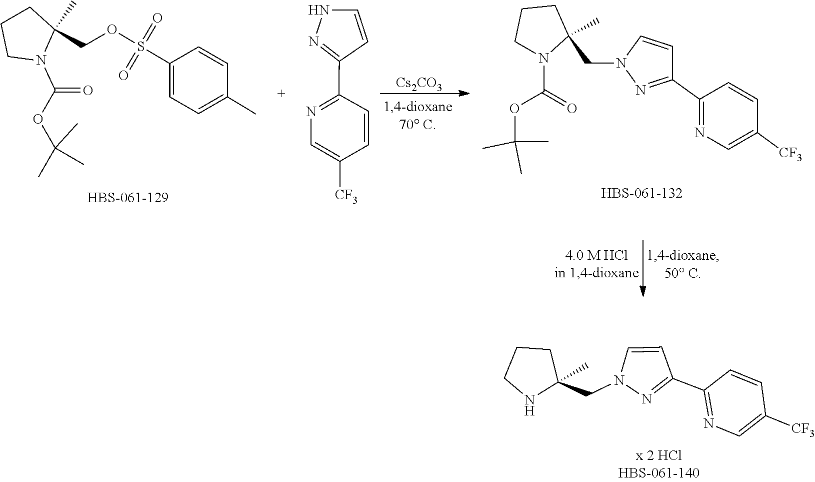

General Synthesis of HBS-061-129: The alcohol (3.09 g, 14.3 mmol) was dissolved in anhydrous DCM (30 mL). DIPEA (3.75 mL, 21.5 mmol) and DMAP (2.63 g, 21.5 mmol) were added sequentially, and the mixture was stirred in an ice bath for 1 h. The p-TsCl (3.01 g, 15.8 mmol) was added in portions over 20 min and the ice bath were allowed to slowly warm to RT 24 h. The reagents were added to consume the starting material during the reaction. The LCMS data shows product formation m/z 314, 270. The reaction mixture was treated with water and the product was extracted with DCM. The DCM layer was dried over Na₂SO₄, filtered, and evaporated. The crude product was purified by column chromatography, mobile phase: Hexane:EtOAc gradient. Obtained 3.21 g of crystalline white solid (Yield=60.4%). MS (ESI) mass calcd. for C₁₈H₂₇NO₅S, 369.2; m/z found 314.1, 270.1 [M+H]⁺.

Step 1: Synthesis of HBS-061-132: HBS-061-129 (1.034 g, 2.80 mmol) and the pyrazole (0.716 g, 3.36 mmol) were dissolved in anhydrous dioxane (20 mL). The Cs₂CO₃ (1.82 g, 5.58 mmol) was added, and the reaction mixture was heated to 70° C. with vigorous stirring. LCMS showed high conversion to product as an 82:18 isomer mixture with m/z 411 after 40 h. The reaction mixture was cooled to RT, treated with water, and extracted with EtOAc. The EtOAc layer was dried over Na₂SO₄, filtered, and evaporated. The crude product was purified by column chromatography, mobile phase: Hexane:EtOAc gradient. Obtained 0.39 g of the major isomer as a colorless oil (Yield=33.7%). MS (ESI) mass calcd. for C₂₀H₂₅F₃N₄O₂, 410.2; m/z found 411.2 [M+H]⁺.

Step 2: Synthesis of HBS-061-140: Compound HBS-061-132 (0.387 g, 0.943 mmol) was dissolved in anhydrous dioxane (7 mL). The 4.0 M HCl in dioxane (1.2 mL, 4.8 mmol) was added and the reaction was stirred vigorously at 50° C. for 21 h. LCMS showed complete conversion to product m/z 311. The reaction mixture was cooled to RT and concentrated to afford a glass. The sample was dissolved in MeOH, concentrated, and dried in a vacuum oven to afford 0.38 g of a glass (Yield Quant.). MS (ESI) mass calcd. for C₁₅H₁₇F₃N₄, 310.2; m/z found 311.2 [M+H]⁺.

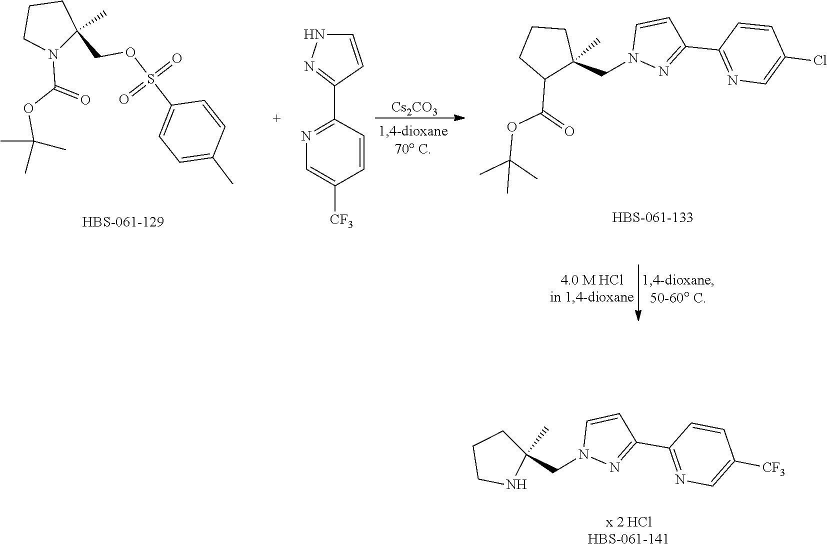

Step 1: Synthesis of HBS-061-133: Compound HBS-061-129 (1.050 g, 2.84 mmol) and the pyrazole (0.612 g, 3.41 mmol) were dissolved in anhydrous dioxane (20 mL). The Cs₂CO₃ (1.85 g, 5.68 mmol) was added, and the reaction mixture was heated to 70° C. with vigorous stirring. LCMS showed high conversion to product as an 80:20 isomer mixture with m/z 377 after 40 h. The reaction mixture was cooled to RT, treated with water, and extracted with EtOAc. The EtOAc layer was dried over Na₂SO₄, filtered, and evaporated. The crude product was purified by column chromatography, mobile phase: Hexane:EtOAc gradient. Obtained 0.35 g of the major isomer as a colorless oil (Yield=32.3%). MS (ESI) mass calcd. for C₁₉H₂₅ClN₄O₂, 376.2; m/z found 377.2 [M+H]⁺.

Step 2: Synthesis of HBS-061-141: Compound HBS-061-133 (0.346 g, 0.918 mmol) was dissolved in anhydrous dioxane (7 mL). The 4.0 M HCl in dioxane (1.15 mL, 4.6 mmol) was added and the reaction was stirred vigorously at 50° C. for 24 h followed by 3 h at 60° C. LCMS showed complete conversion to product m/z 277. The reaction mixture was cooled to RT, filtered, and washed with hexanes to afford 0.27 g of powdery white solid (Yield=85.3%). MS (ESI) mass calcd. for C₁₄H₁₇ClN₄, 276.1; m/z found 277.1 [M+H]⁺.

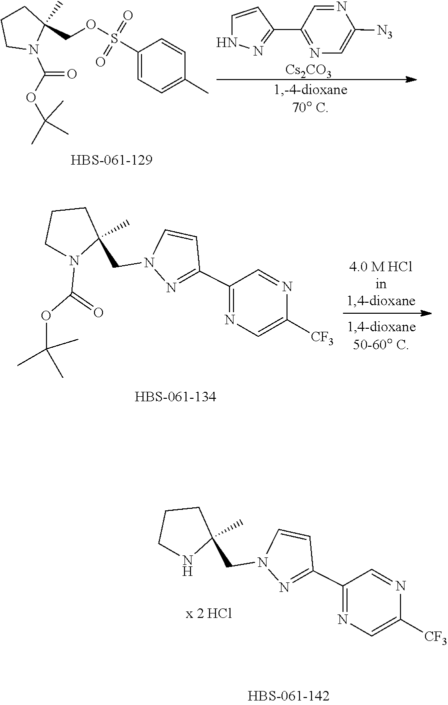

Step 1: Synthesis of HBS-061-134: Compound HBS-061-129 (1.011 g, 2.74 mmol) and the pyrazole (0.612 g, 3.41 mmol) were dissolved in anhydrous dioxane (20 mL). The Cs₂CO₃ (1.78 g, 5.46 mmol) was added, and the reaction mixture was heated to 70° C. with vigorous stirring. LCMS showed high conversion to product as an 88:12 isomer mixture with m/z 412 after 40 h. The reaction mixture was cooled to RT, treated with water, and extracted with EtOAc. The EtOAc layer was dried over Na₂SO₄, filtered, and evaporated. The crude product was purified by column chromatography, mobile phase: Hexane:EtOAc gradient. Obtained 0.30 g of the major isomer as a colorless oil (Yield=26.9%). MS (ESI) mass calcd. for C₁₉H₂₄F₃N₅O₂, 411.2; m/z found 412.1 [M+H]⁺.

Step 2: Synthesis of HBS-061-142: Compound HBS-061-134 (0.303 g, 0.736 mmol) was dissolved in anhydrous dioxane (7 mL). The 4.0 M HCl in dioxane (0.92 mL, 3.7 mmol) was added and the reaction was stirred vigorously at 50° C. for 24 h followed by 3 h at 60° C. LCMS showed complete conversion to product m/z 312. Upon cooling a precipitate formed. The reaction mixture was filtered and washed with Hexane to afford 0.23 g of powdery white solid (Yield=79.9%). MS (ESI) mass calcd. for C₁₄H₁₆F₃N₅, 311.1; m/z found 312.2 [M+H]⁺.

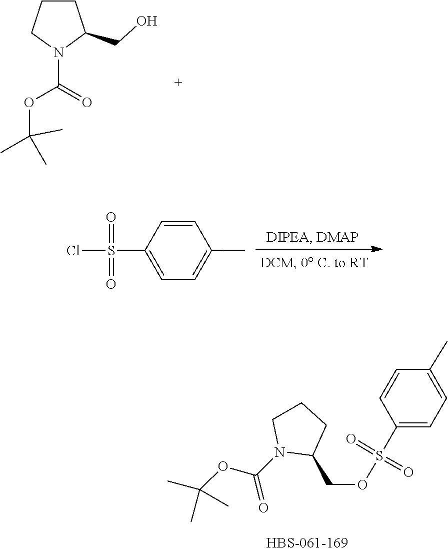 [SMILES: CC(C)(C)OC(=O)N1CCC[C@H]1CO ; Cc1ccc(S(=O)(=O)Cl)cc1 ; Cc1ccc(S(=O)(=O)OC[C@@H]2CCCN2C(=O)OC(C)(C)C)cc1]

Synthesis of HBS-061-169: The alcohol (3.02 g, 15.0 mmol) was dissolved in anhydrous CH₂Cl₂ (32 mL). DIPEA (3.92 mL, 22.5 mmol) and DMAP (2.75 g, 22.5 mmol) were added sequentially, and the mixture was stirred in an ice bath for 1 h. p-TsCl (3.43 g, 15.8 mmol) was added in portions over 10 min and the ice bath were allowed to slowly warm to RT. After 24 h at RT LCMS showed complete conversion to product with m/z 300, 256. The reaction mixture was treated with water and the product was extracted with DCM. The DCM layer was dried over Na₂SO₄, filtered, and evaporated. The crude product was purified by column chromatography, mobile phase: Hexane:EtOAc gradient. Obtained 4.91 g of colorless oil (Yield=92.0%). MS (ESI) mass calcd. for C₁₇H₂₅NO₅S, 355.2; m/z found 300.0, 256.0 [M+H]⁺.

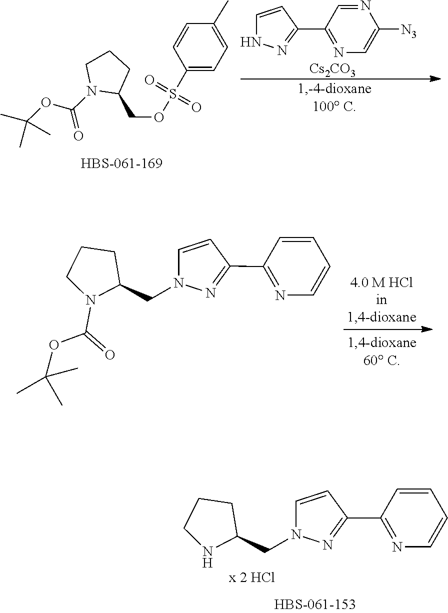

Step 1: Synthesis of HBS-061-146: Compound HBS-061-169 (1.533 g, 4.31 mmol) and the pyrazole (0.750 g, 5.17 mmol) were dissolved in anhydrous dioxane (30 mL). The Cs₂CO₃ (2.81 g, 5.17 mmol) was added, and the reaction mixture was heated to 100° C. with vigorous stirring. LCMS showed high conversion to product as an 83:17 isomer mixture with m/z 329 after 24 h. The reaction mixture was cooled to RT, treated with water, and extracted with EtOAc. The EtOAc layer was dried over Na₂SO₄, filtered, and evaporated. The crude product was purified by column chromatography, mobile phase: Hexane:EtOAc gradient. Obtained 1.06 g of the major isomer as a colorless oil (Yield=75.0%). MS (ESI) mass calcd. for C₁₈H₂₄N₄O₂, 328.2; m/z found 329.2 [M+H]⁺.

Step 2: Synthesis of HBS-061-153: Compound HBS-061-146 (1.061 g, 3.23 mmol) was dissolved in anhydrous dioxane (12 mL). The 4.0 M HCl in dioxane (4.04 mL, 16.2 mmol) was added and the reaction was stirred vigorously at 60° C. for 18 h. LCMS showed complete conversion to product m/z 229. The reaction mixture was cooled to RT, filtered, and washed with hexanes to afford 0.97 g of powdery white solid (Yield=100%). MS (ESI) mass calcd. for C₁₃H₁₆N₄, 228.1; m/z found 229.1 [M+H]⁺.

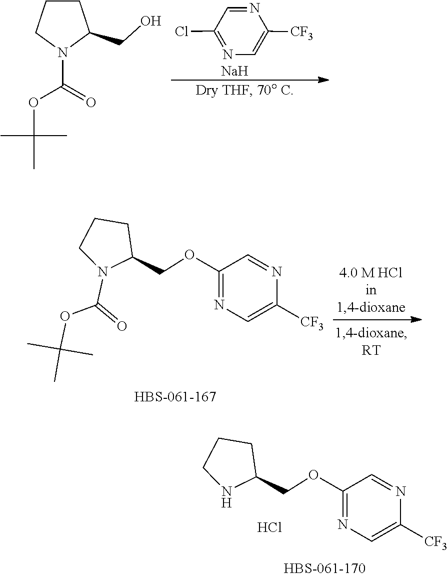

Step 1: Synthesis of HBS-061-167: The alcohol (1.41 g, 7.0 mmol) was dissolved in anhydrous THF (10 mL). The 60% NaH (0.42 g, 10.5 mmol) was added in one portion and after 15 minutes the chloropyrimidine (1.28 g, 7.00 mmol) was added as a solution in DMF (6 mL). The reaction mixture was heated to 70° C. LCMS showed product formation after 3 h with m/z 292, 248. The reaction mixture was cooled to RT, treated with water, and extracted with EtOAc. The EtOAc layer was dried over Na₂SO₄, filtered, and evaporated. The crude product was purified by column chromatography, mobile phase: Hexane:EtOAc gradient. Obtained 0.13 g of the product as a colorless oil (Yield=5.3%). MS (ESI) mass calcd. for C₁₅H₂₀F₃N₃O₃, 347.2; m/z found 292.0, 248.1 [M+H]⁺.

Step 2: Synthesis of HBS-061-170: Compound HBS-061-167 (0.129 g, 0.371 mmol) was dissolved in anhydrous dioxane (2 mL). The 4.0 M HCl in dioxane (0.47 mL, 1.88 mmol) was added and the reaction was stirred vigorously at RT for 96 h forming a suspension. LCMS showed complete conversion to product m/z 248. The reaction mixture was filtered and washed with hexanes to afford 67 mg of white solid (yield=63.5%). MS (ESI) mass calcd. for C₁₀H₁₂F₃N₃O, 247.1; m/z found 248.1 [M+H]⁺.

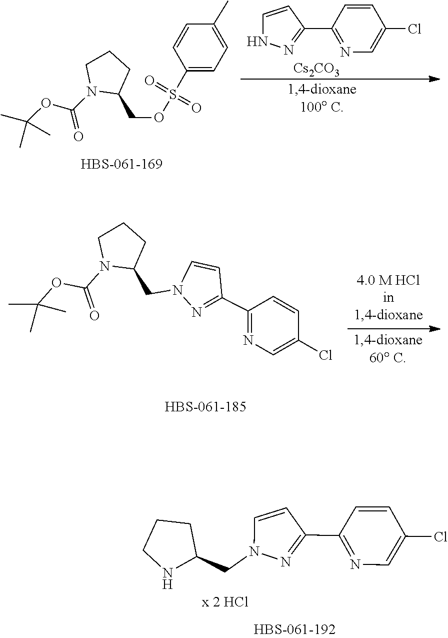

Step 1: Synthesis of HBS-061-185: Compound HBS-061-169 (0.695 g, 1.96 mmol) and the pyrazole (0.422 g, 2.35 mmol) were dissolved in anhydrous dioxane (15 mL). The Cs₂CO₃ (1.27 g, 3.90 mmol) was added, and the reaction mixture was heated to 100° C. with vigorous stirring. LCMS showed high conversion to product with isomer mixture m/z 363 after 42 h. The reaction mixture was cooled to RT, treated with water, and extracted with EtOAc. The EtOAc layer was dried over Na₂SO₄, filtered, and evaporated. The crude product was purified by column chromatography, mobile phase: Hexane:EtOAc gradient. Obtained 0.18 g of the major isomer as a colorless oil (Yield=24.9%). MS (ESI) mass calcd. for C₁₈H₂₃ClN₄O₂, 362.2; m/z found 363.1 [M+H]⁺.

Step 2: Synthesis of HBS-061-192: Compound HBS-061-185 (0.177 g, 0.488 mmol) was dissolved in anhydrous dioxane (10 mL). The 4.0 M HCl in dioxane (1.22 mL, 4.88 mmol) was added and the reaction was stirred vigorously at 60° C. for 64 h. LCMS showed complete conversion to product m/z 263. The reaction mixture was cooled to RT, filtered, and washed with Hexane to afford 0.16 g of white solid (Yield=97.7%). MS (ESI) mass calcd. for C₁₃H₁₅ClN₄, 262.1; m/z found 263.1 [M+H]⁺.

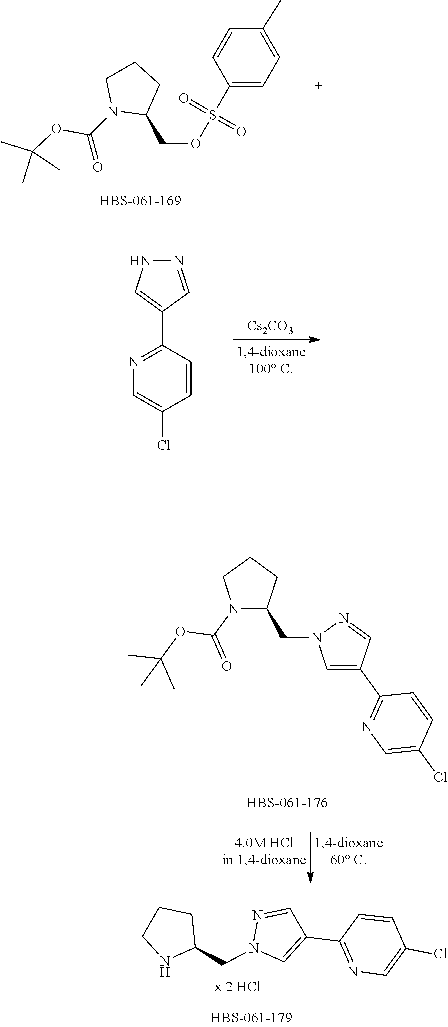

Step 1: Synthesis of HBS-061-176: Compound HBS-061-169 (0.704 g, 1.98 mmol) and the pyrazole (0.427 g, 2.38 mmol) were dissolved in anhydrous dioxane (20 mL). The Cs₂CO₃ (1.29 g, 3.96 mmol) was added, and the reaction mixture was heated to 100° C. with vigorous stirring. LCMS showed high conversion to product with m/z 363 after 64 h. The reaction mixture was cooled to RT, treated with water, and extracted with EtOAc. The EtOAc layer was dried over Na₂SO₄, filtered, and evaporated. The crude product was purified by column chromatography, mobile phase: Hexane:EtOAc gradient. Obtained 0.64 g of the product as a colorless oil (Yield=89.3%). MS (ESI) mass calcd. for C₁₈H₂₃ClN₄O₂, 362.2; m/z found 363.1 [M+H]⁺.

Step 2: Synthesis of HBS-061-179: Compound HBS-061-176 (0.642 g, 1.77 mmol) was dissolved in anhydrous dioxane (20 mL). The 4.0 M HCl in dioxane (2.21 mL, 8.84 mmol) was added and the reaction was stirred vigorously at 60° C. for 18 h. LCMS showed complete conversion to product m/z 263. The reaction mixture was cooled to RT, filtered, and washed with Hexane to afford 0.54 g of powdery white solid (Yield=91.6%). MS (ESI) mass calcd. for C₁₃H₁₅ClN₄, 262.1; m/z found 263.1 [M+H]⁺.

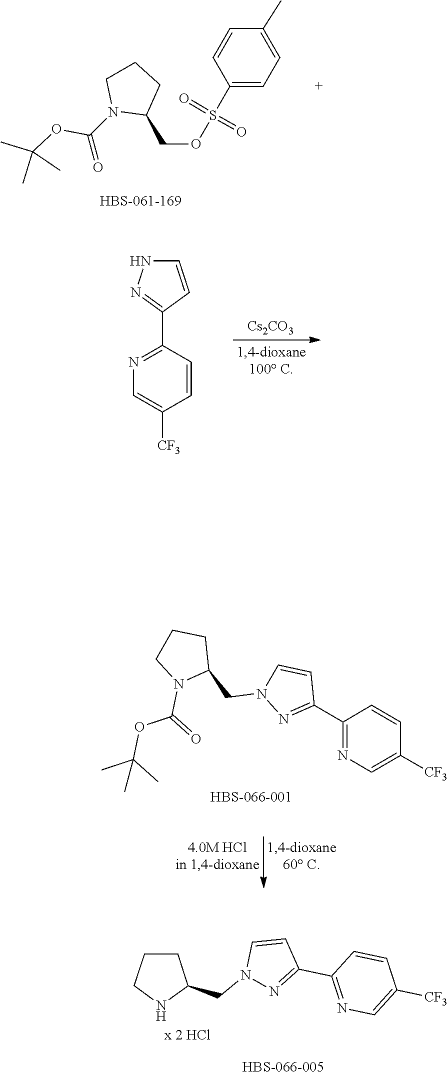

Step 1: Synthesis of HBS-066-001: HBS-061-169 (0.614 g, 1.73 mmol) and the pyrazole (0.441 g, 2.07 mmol) were dissolved in anhydrous dioxane (20 mL). The Cs₂CO₃ (1.12 g, 3.44 mmol) was added, and the reaction mixture was heated to 100° C. with vigorous stirring. LCMS showed high conversion to product as an 88:12 isomer mixture with m/z 397 after 64 h. The reaction mixture was cooled to RT, treated with water, and extracted with EtOAc. The EtOAc layer was dried over Na₂SO₄, filtered, and evaporated. The crude product was purified by column chromatography, mobile phase: Hexanes:EtOAc gradient. Obtained 0.51 g of the major isomer as a waxy white solid (Yield=74.9%). MS (ESI) mass calcd. for C₁₉H₂₃F₃N₄O₂, 396.2; m/z found 397.1 [M+H]⁺.

Step 2: Synthesis of HBS-066-005: Compound HBS-066-001 (0.513 g, 1.29 mmol) was dissolved in anhydrous dioxane (20 mL). The 4.0 M HCl in dioxane (1.62 mL, 6.48 mmol) was added and the reaction was stirred vigorously at 60° C. for 90 h. LCMS showed complete conversion to product m/z 297. The reaction mixture was cooled to RT, filtered, and washed with Hexane to afford 0.36 g of powdery white solid (Yield=76.0%). MS (ESI) mass calcd. for C₁₄H₁₅F₃N₄, 296.1; m/z found 297.1 [M+H]⁺.

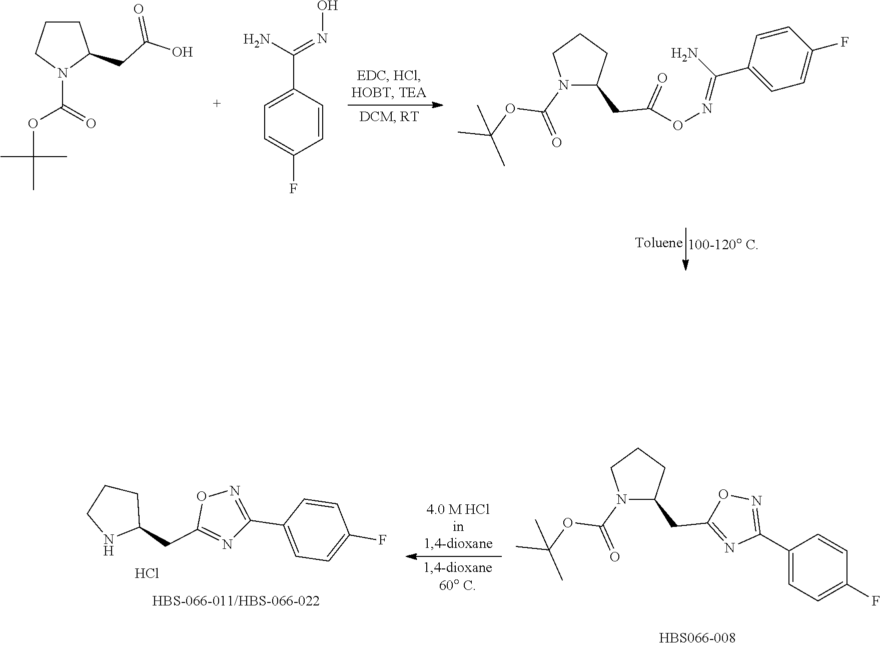 [SMILES: CC(C)(C)OC(=O)N1CCC[C@H]1CC(=O)O ; N/C(=N\O)c1ccc(F)cc1 ; CC(C)(C)OC(=O)N1CCC[C@H]1CC(=O)O/N=C(\N)c1ccc(F)cc1 ; Fc1ccc(-c2noc(C[C@@H]3CCCN3)n2)cc1 ; Cl ; CC(C)(C)OC(=O)N1CCC[C@H]1Cc1nc(-c2ccc(F)cc2)no1]

Step 1: Synthesis of HBS-061-186: The carboxylic acid (1.720 g, 7.50 mmol) was dissolved in CH₂Cl₂ (40 mL). Introduced sequentially the hydroxyl amidine (0.771 g, 5.00 mmol), HOBT (1.35 g, 10.0 mmol), EDC (1.917 g, 10.0 mmol), and TEA (3.50 mL, 25.0 mmol), and stirred at RT. LCMS showed conversion to the acyl intermediate was complete after 24 h with m/z 366. The acyl intermediate was isolated by extractive workup between EtOAc and water affording a viscous yellow oil (2.44 g). The oil was dissolved in DCM (20 mL), toluene (30 mL) was added, and the reaction was heated in an open flask to 100° C. LCMS showed complete conversion after 21 h forming product with m/z 348, 292, 248. Upon cooling the reaction mix was dissolved in EtOAc and extracted with aq. sat. NaHCO₃. The EtOAc layer was dried over Na₂SO₄, filtered, and evaporated. The crude product was purified by column chromatography, mobile phase: Hexane:EtOAc gradient. Obtained 0.717 g of colorless oil (Yield=41.0%). MS (ESI) mass calcd. for C₁₈H₂₂FN₃O₃, 347.2; m/z found 348.1, 292.0, 248.0 [M+H]⁺.

Step 2: Synthesis of HBS-066-011/HBS-066-022: Compound HBS-066-008 (0.717 g, 2.06 mmol) was dissolved in anhydrous dioxane (20 mL). The 4.0 M HCl in dioxane (3.29 mL, 13.2 mmol) was added and the reaction was stirred vigorously at 60° C. for 72 h. LCMS showed complete conversion to product m/z 248. The reaction mixture was cooled to RT, filtered, and washed with Hexane to afford 0.53 g of powdery white solid (Yield=90.2%). MS (ESI) mass calcd. for C₁₃H₁₄FN₃O, 247.1; m/z found 248.1 [M+H]⁺.

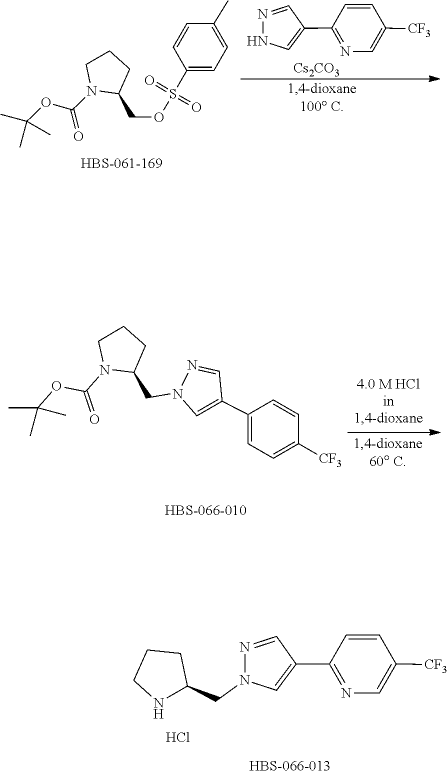

Step 1: Synthesis of HBS-066-010: Compound HBS-061-169 (0.547 g, 1.54 mmol) and the pyrazole (0.392 g, 1.85 mmol) were dissolved in anhydrous dioxane (20 mL). The Cs₂CO₃ (1.00 g, 3.07 mmol) was added, and the reaction mixture was heated to 100° C. with vigorous stirring. LCMS showed high conversion to product with m/z 396 after 48 h. The reaction mixture was cooled to RT, treated with water, and extracted with EtOAc. The EtOAc layer was dried over Na₂SO₄, filtered, and evaporated. The crude product was purified by column chromatography, mobile phase: Hexane:EtOAc gradient. Obtained 0.47 g of the product as a colorless oil (Yield=77.7%). MS (ESI) mass calcd. for C₂₀H₂₄F₃N₃O₂, 395.2; m/z found 396.1 [M+H]⁺.

Step 2: Synthesis of HBS-066-013: Compound HBS-066-010 (0.473 g, 1.20 mmol) was dissolved in anhydrous dioxane (20 mL). The 4.0 M HCl in dioxane (1.50 mL, 6.00 mmol) was added and the reaction was stirred vigorously at 60° C. for 66 h. LCMS showed complete conversion to product m/z 296. The reaction mixture was cooled to RT, filtered, and washed with Hexane to afford 0.37 g of powdery white solid (Yield=84.0%). MS (ESI) mass calcd. for C₁₅H₁₆F₃N₃, 295.1; m/z found 296.1 [M+H]⁺.

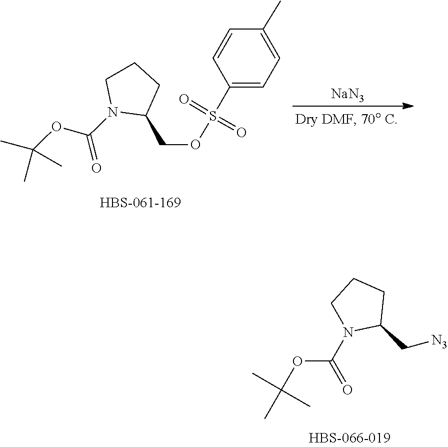

Synthesis of HBS-066-019: HBS-061-169 (1.179 g, 3.32 mmol) was dissolved in anhydrous DMF (20 mL). The NaN₃ (0.323 g, 4.97 mmol) was added, and the reaction mixture was heated to 70° C. with vigorous stirring. LCMS showed high conversion to product with m/z 171, 127 after 24 h. The reaction mixture was cooled to RT, treated with water, and extracted with EtOAc. The EtOAc layer was washed with brine, dried over Na₂SO₄, filtered, and evaporated. The crude product was purified by column chromatography, mobile phase: Hexane:EtOAc gradient. Obtained 0.630 g of the product as a colorless oil (Yield=83.9%). MS (ESI) mass calcd. for C₁₀H₁₈N₄O₂, 226.2; m/z found 171.1, 127.1 [M+H]⁺.

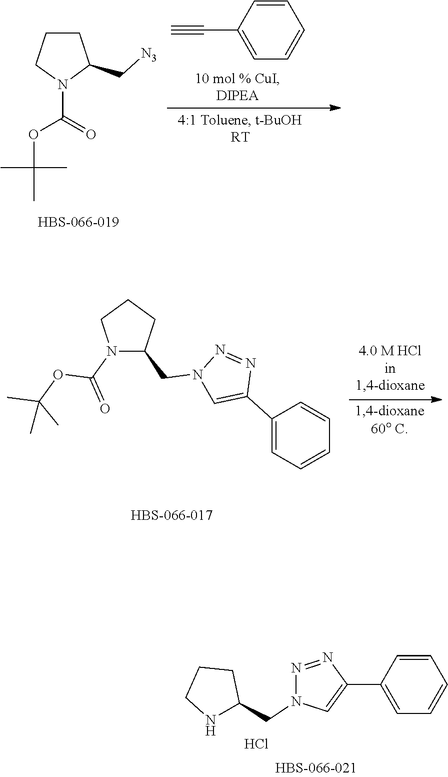

Step 1: Synthesis of HBS066-017: Compound HBS-066-019 (0.177 g, 0.782 mmol) was dissolved in a mixture of toluene and t-BuOH (4:1 v/v mL). Added sequentially the acetylene (0.103 mL, 0.938 mmol), CuI (15 mg, 0.078 mmol), and DIPEA (0.272 mL, 1.56 mmol). The reaction mixture was stirred vigorously at RT. LCMS showed high conversion to product with m/z 329.0 after 42 h. The reaction mixture was evaporated, and the crude product was purified by column chromatography, mobile phase: Hexane:EtOAc gradient. Obtained 0.159 g of the product as a white solid (Yield=62.0%). MS (ESI) mass calcd. for C₁₈H₂₄N₄O₂, 328.2; m/z found 329.2 [M+H]⁺.

Step 2: Synthesis of HBS-066-021: Compound HBS-066-017 (0.159 g, 0.484 mmol) was dissolved in anhydrous dioxane (10 mL). The 4.0 M HCl in dioxane (0.81 mL, 3.24 mmol) was added and the reaction was stirred vigorously at 60° C. for 72 h forming a suspension. LCMS showed complete conversion to product m/z 229. The reaction mixture was cooled to RT, filtered, and washed with Hexane. The sample was dissolved in MeOH, concentrated, and dried in a vacuum oven to afford 0.14 g of off white waxy solid (yield=95.3%). MS (ESI) mass calcd. for C₁₃H₁₆N₄, 228.1; m/z found 229.1 [M+H]⁺.

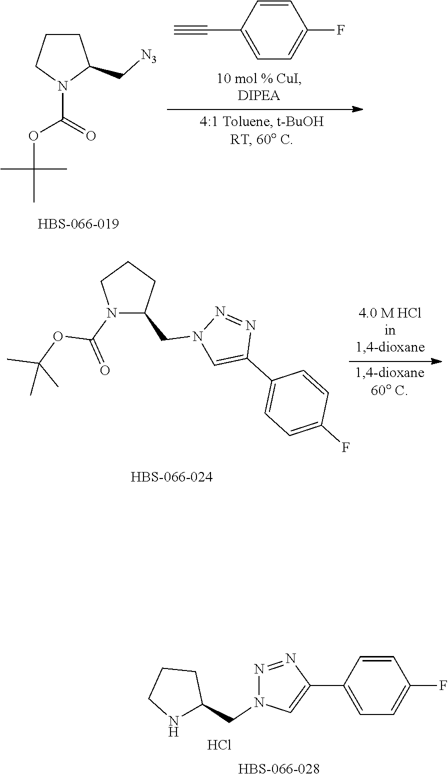

Step 1: Synthesis of HBS-066-024: Compound HBS066-019 (0.315 g, 1.39 mmol) was dissolved in a mixture of toluene and t-BuOH (6:1.5, v/v mL). Added sequentially the acetylene (0.200 g, 1.66 mmol), CuI (28 mg, 0.147 mmol), and DIPEA (0.485 mL, 2.78 mmol). The reaction mixture was stirred vigorously at RT. LCMS showed incomplete conversion to product after 42 h. Heated to 60° C. to achieve high conversion after 2.5 h forming product with m/z 347. The reaction mixture was evaporated, and the crude product was purified by column chromatography, mobile phase: hexanes/EtOAc gradient. Obtained 0.32 g of the product as a white solid (Yield=66.6%). MS (ESI) mass calcd. for C₁₈H₂₃FN₄O₂, 346.2; m/z found 347.1 [M+H]⁺.

Step 2: Synthesis of HBS-066-028: Compound HBS-066-024 (0.321 g, 0.927 mmol) was dissolved in anhydrous dioxane (20 mL). The 4.0 M HCl in dioxane (2.32 mL, 9.27 mmol) was added and the reaction was stirred vigorously at 60° C. for 42 h. LCMS showed complete conversion to product m/z 247. The reaction mixture was cooled to RT, filtered, and washed with hexanes to obtain a white waxy solid that adhered to the filter paper. The sample was dissolved in MeOH, concentrated, and dried in a vacuum oven to afford 0.32 g of white waxy solid (Yield Quant.). MS (ESI) mass calcd. for C₁₃H₁₅FN₄, 246.1; m/z found 247.1 [M+H]⁺.

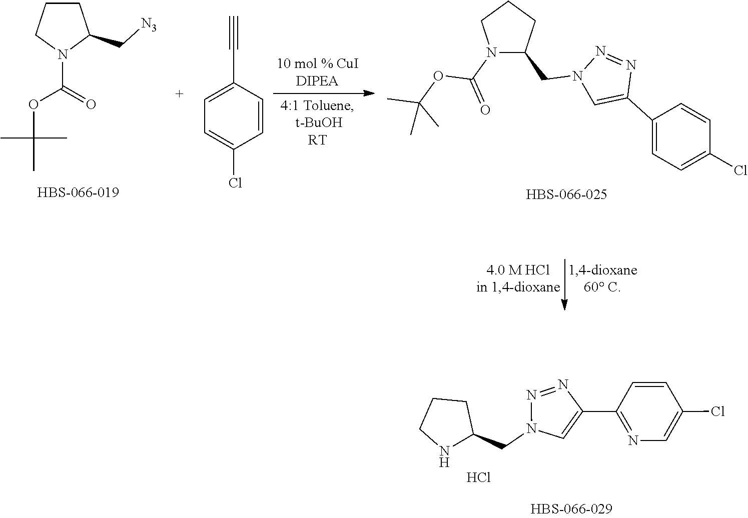

Step 1: Synthesis of HBS-066-025: Compound HBS-066-019 (0.315 g, 1.39 mmol) was dissolved in a mixture of toluene and t-BuOH (6:1.5, v/v mL). Added sequentially the acetylene (0.228 g, 1.67 mmol), CuI (28 mg, 0.147 mmol), and DIPEA (0.485 mL, 2.78 mmol). The reaction mixture was stirred vigorously at RT. LCMS showed incomplete conversion to product after 42 h. Heated to 60° C. to achieve high conversion after 2.5 h forming product with m/z 363.

The reaction mixture was evaporated, and the crude product was purified by column chromatography, mobile phase: Hexane:EtOAc gradient. Obtained 0.312 g of the product as a white solid (Yield=61.8%). MS (ESI) mass calcd. for C₁₈H₂₃ClN₄O₂, 362.2; m/z found 363.1 [M+H]⁺.

Step 2: Synthesis of HBS-066-029: Compound HBS-066-025 (0.31 g, 0.860 mmol) was dissolved in anhydrous dioxane (20 mL). The 4.0 M HCl in dioxane (2.15 mL, 8.60 mmol) was added and the reaction was stirred vigorously at 60° C. for 42 h forming a suspension. LCMS showed complete conversion to product m/z 263. The reaction mixture was cooled to RT, filtered, and washed with Hexane to afford 0.260 g of white solid (Yield=90.1%). MS (ESI) mass calcd. for C₁₃H₁₅ClN₄, 262.1; m/z found 263.1 [M+H]⁺.

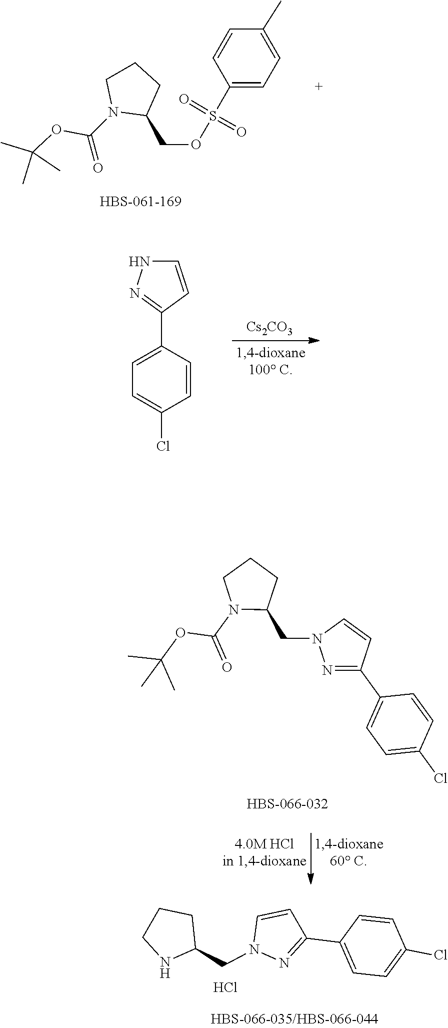

Step 1: Synthesis of HBS-066-032: HBS-061-169 (1.01 g, 2.83 mmol) and the pyrazole (0.607 g, 3.40 mmol) were dissolved in anhydrous dioxane (25 mL). The Cs₂CO₃ (1.85 g, 5.68 mmol) was added, and the reaction mixture was heated to 100° C. with vigorous stirring. LCMS showed high conversion to product as a 92:8 isomer mixture with m/z 362 after 24 h. The reaction mixture was cooled to RT, treated with water, and extracted with EtOAc. The EtOAc layer was dried over Na₂SO₄, filtered, and evaporated. The crude product was purified by column chromatography, mobile phase: Hexane:EtOAc gradient. Obtained 0.518 g of the major isomer as a colorless oil (Yield=50.5%). MS (ESI) mass calcd. for C₁₉H₂₄ClN₃O₂, 361.2; m/z found 362.1 [M+H]⁺.

Step 2: Synthesis of HBS-066-035/HBS-066-044: Compound HBS-066-032 (0.518 g, 1.43 mmol) was dissolved in anhydrous dioxane (30 mL). The 4.0 M HCl in dioxane (3.58 mL, 14.3 mmol) was added and the reaction was stirred vigorously at 60° C. for 66 h forming a suspension. LCMS showed complete conversion to product m/z 262. The reaction mixture was cooled to RT, filtered, and washed with Hexane to afford 0.395 g of powdery white solid (Yield=82.4%). MS (ESI) mass calcd. for C₁₄H₁₆ClN₃, 261.1; m/z found 262.1 [M+H]⁺.

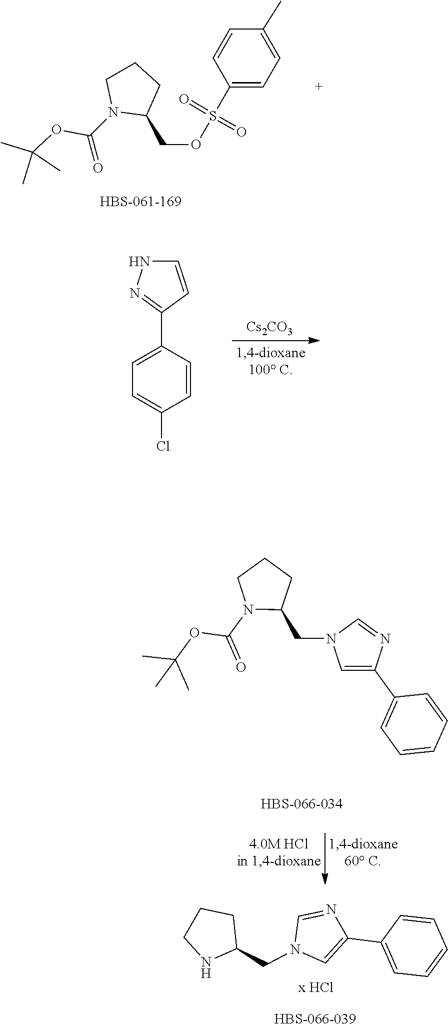

Step 1: Synthesis of HBS066-034: HBS-061-169 (0.499 g, 1.40 mmol) and the pyrazole (0.243 g, 1.68 mmol) were dissolved in anhydrous dioxane (12 mL). The Cs₂CO₃ (0.915 g, 2.81 mmol) was added, and the reaction mixture was heated to 100° C. with vigorous stirring. LCMS showed high conversion to product with isomer mixture m/z 328 after 90 h. The reaction mixture was cooled to RT, treated with water, and extracted with EtOAc. The EtOAc layer was dried over Na₂SO₄, filtered, and evaporated. TLC (95:5, DCM, MeOH) showed the isomers separating. The crude product was purified by column chromatography, mobile phase: DCM:MeOH gradient. Obtained 0.253 g of the major isomer as a yellow oil (Yield=55.0%). MS (ESI) mass calcd. for C₁₉H₂₅N₃O₂, 327.2; m/z found 328.2 [M+H]⁺.

Step 2: Synthesis of HBS-066-039: Compound HBS-066-034 (0.253 g, 7.73 mmol) was dissolved in anhydrous dioxane (20 mL). The 4.0 M HCl in dioxane (1.93 mL, 4.88 mmol) was added and the reaction was stirred vigorously at 60° C. for 27 h forming a suspension. LCMS showed complete conversion to product m/z 228. The reaction mixture was cooled to RT, filtered, and washed with Hexane. The sample was dissolved in MeOH, concentrated, and dried in a vacuum oven to afford 0.168 g of white crystalline wax (Yield=72.4%). MS (ESI) mass calcd. for C₁₄H₁₇N3, 227.1; m/z found 228.1 [M+H]⁺.

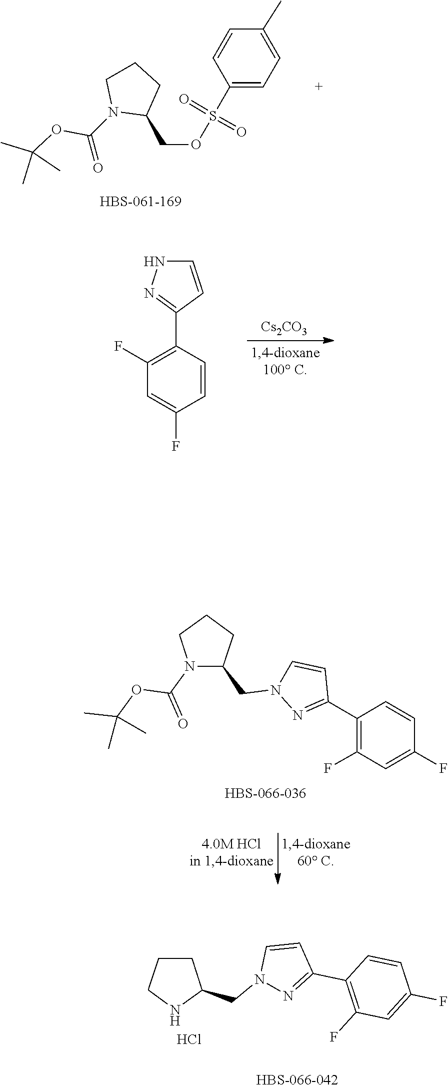

Step 1: Synthesis of HBS-066-036: Compound HBS-061-169 (0.978 g, 2.75 mmol) and the pyrazole (0.545 g, 3.02 mmol) were dissolved in anhydrous dioxane (25 mL). The Cs₂CO₃ (1.79 g, 5.49 mmol) was added, and the reaction mixture was heated to 100° C. with vigorous stirring. LCMS showed high conversion to product as a 96:4 isomer mixture with m/z 364 after 64 h. The reaction mixture was cooled to RT, treated with water, and extracted with EtOAc. The EtOAc layer was dried over Na₂SO₄, filtered, and evaporated. The crude product was purified by column chromatography, mobile phase: Hexane:EtOAc gradient. Obtained 0.682 g of the major isomer as a colorless oil (Yield=68.2%). MS (ESI) mass calcd. for C₁₉H₂₃F₂N₃O₂, 363.2; m/z found 364.2 [M+H]⁺.

Step 2: Synthesis of HBS-066-042: Compound HBS-066-036 (0.682 g, 1.88 mmol) was dissolved in anhydrous dioxane (20 mL). The 4.0 M HCl in dioxane (2.35 mL, 9.40 mmol) was added and the reaction was stirred vigorously at 60° C. for 66 h forming a suspension. LCMS showed complete conversion to product m/z 264. The reaction mixture was cooled to RT, filtered, and washed with hexanes to obtain a film that adhered to the filter paper. The sample was dissolved in MeOH, concentrated, and dried in a vacuum oven to afford 0.55 g of off-white wax (Yield=87.2%). MS (ESI) mass calcd. for C₁₄H₁₅F₂N₃, 263.1; m/z found 264.2 [M+H]⁺.

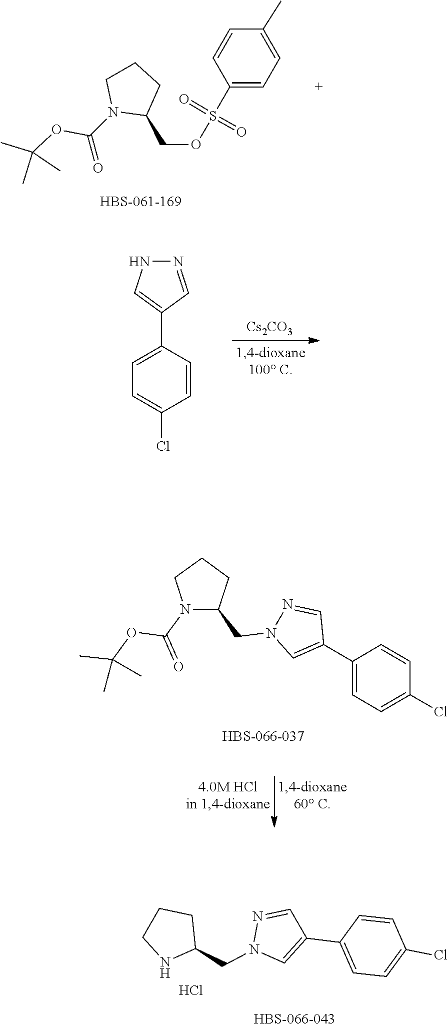

Step 1: Synthesis of HBS-066-037: Compound HBS-061-169 (0.963 g, 2.71 mmol) and the pyrazole (0.532 g, 2.98 mmol) were dissolved in anhydrous dioxane (25 mL). The Cs₂CO₃ (1.76 g, 5.40 mmol) was added, and the reaction mixture was heated to 100° C. with vigorous stirring. LCMS showed high conversion to product with m/z 362 after 64 h. The reaction mixture was cooled to RT, treated with water, and extracted with EtOAc. The EtOAc layer was dried over Na₂SO₄, filtered, and evaporated. The crude product was purified by column chromatography, mobile phase: Hexane:EtOAc gradient. Obtained 0.65 g of the major isomer as a yellow oil (Yield=66.4%). MS (ESI) mass calcd. for C₁₉H₂₄ClN₃O₂, 361.2; m/z found 362.1 [M+H]⁺.

Step 2: Synthesis of HBS-066-043: Compound HBS066-037 (0.651 g, 1.80 mmol) was dissolved in anhydrous dioxane (20 mL). The 4.0 M HCl in dioxane (2.25 mL, 9.00 mmol) was added and the reaction was stirred vigorously at 60° C. for 66 h forming a suspension. LCMS showed complete conversion to product m/z 262. The reaction mixture was cooled to RT, filtered, and washed with hexanes to obtain a film that adhered to the filter paper. The sample was dissolved in MeOH, concentrated, and dried in a vacuum oven to afford 0.54 g of beige wax (Yield=88.9%). MS (ESI) mass calcd. for C₁₄H₁₆ClN₃, 261.1; m/z found 262.1 [M+H]⁺.

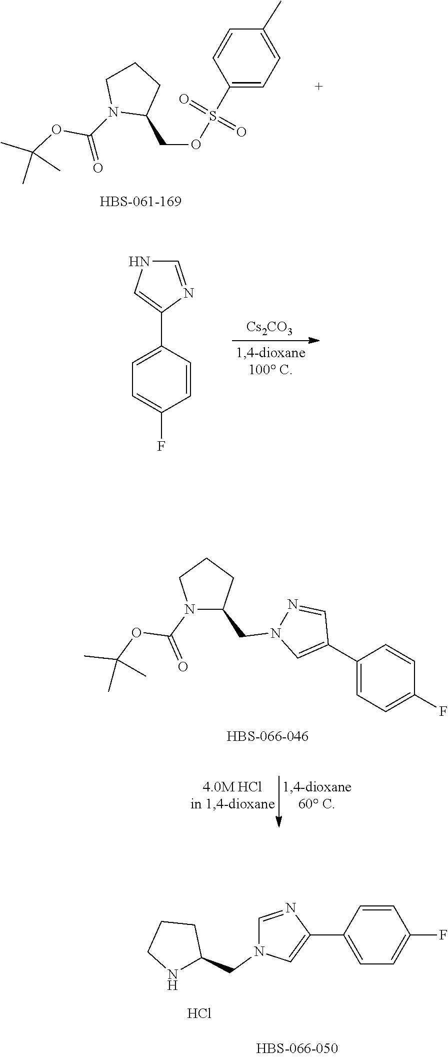

Step 1: Synthesis of HBS-066-046: Compound HBS-061-169 (1.355 g, 3.81 mmol) and the pyrazole (0.680 g, 4.19 mmol) were dissolved in anhydrous dioxane (40 mL). The Cs₂CO₃ (2.48 g, 7.61 mmol) was added, and the reaction mixture was heated to 100° C. with vigorous stirring.

LCMS showed high conversion to product with isomer mixture m/z 346 after 48 h. The reaction mixture was cooled to RT, treated with water, and extracted with EtOAc. The EtOAc layer was dried over Na₂SO₄, filtered, and evaporated. TLC (95:5 DCM, MeOH) showed the isomers separating. The crude product was purified by column chromatography, mobile phase: DCM:MeOH gradient. Obtained 0.63 g of the major isomer as a yellow oil (Yield=47.9%). MS (ESI) mass calcd. for C₁₉H₂₄FN₃O₂, 345.2; m/z found 346.2 [M+H]⁺.

Step 2: Synthesis of HBS-066-050: Compound HBS-066-046 (0.61 g, 1.78 mmol) was dissolved in anhydrous dioxane (20 mL). The 4.0 M HCl in dioxane (2.22 mL, 8.88 mmol) was added and the reaction was stirred vigorously at 60° C. for 68 h forming a suspension. LCMS showed complete conversion to product m/z 246. The reaction mixture was cooled to RT, filtered, and washed with Hexane to afford 0.478 g of white solid (Yield=84.5%). MS (ESI) mass calcd. for C₁₄H₁₆FN₃, 245.1; m/z found 246.2 [M+H]⁺.

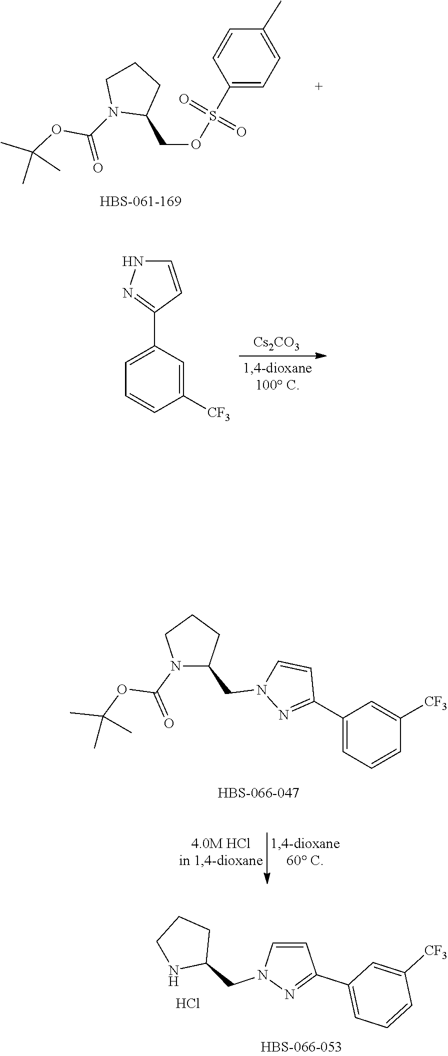

Step 1: Synthesis of HBS-066-047: Compound HBS-061-169 (0.858 g, 2.41 mmol) and the pyrazole (0.564 g, 2.66 mmol) were dissolved in anhydrous dioxane (25 mL). The Cs₂CO₃ (1.57 g, 4.82 mmol) was added, and the reaction mixture was heated to 100° C. with vigorous stirring. LCMS showed high conversion to product as a 92:8 isomer mixture with m/z 396 after 64 h. The reaction mixture was cooled to RT, treated with water, and extracted with EtOAc. The EtOAc layer was dried over Na₂SO₄, filtered, and evaporated. The crude product was purified by column chromatography, mobile phase: hexanes/EtOAc gradient. Obtained 0.70 g of the major isomer as a colorless oil (Yield=73.8%). MS (ESI) mass calcd. for C₂₀H₂₄F₃N₃O₂, 395.2; m/z found 396.2 [M+H]⁺.

Step 2: Synthesis of HBS-066-053: Compound HBS-066-047 (0.704 g, 1.78 mmol) was dissolved in anhydrous dioxane (20 mL). The 4.0 M HCl in dioxane (2.20 mL, 8.80 mmol) was added and the reaction was stirred vigorously at 60° C. for 72 h. LCMS showed complete conversion to product m/z 296. The reaction mixture was cooled to RT, filtered, and washed with Hexane. The sample was dissolved in MeOH, concentrated, and dried in a vacuum oven to afford 0.59 g of off white crusty solid (Yield=90.1%). MS (ESI) mass calcd. for C₁₅H₁₆F₃N₃, 295.1; m/z found 296.2 [M+H]⁺.

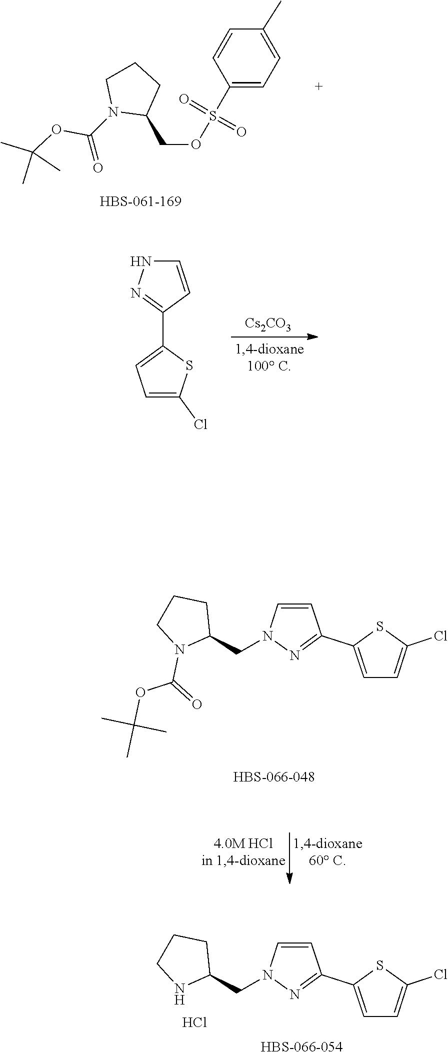

Step 1: Synthesis of HBS-066-048: HBS061-169 (0.866 g, 2.44 mmol) and the pyrazole (0.495 g, 2.68 mmol) were dissolved in anhydrous dioxane (25 mL). The Cs₂CO₃ (1.59 g, 4.88 mmol) was added, and the reaction mixture was heated to 100° C. with vigorous stirring. LCMS showed high conversion to product with isomer mixture m/z 368 after 64 h. The reaction mixture was cooled to RT, treated with water, and extracted with EtOAc. The EtOAc layer was dried over Na₂SO₄, filtered, and evaporated. TLC (2:1 Hexane:EtOAc) showed the isomers separating. The crude product was purified by column chromatography, mobile phase: Hexane:EtOAc gradient. Obtained 0.670 g of the major isomer as a colorless oil (Yield=74.7%). MS (ESI) mass calcd. for C₁₇H₂₂ClN₃O₂S, 367.1; m/z found 368.1 [M+H]⁺.

Step 2: Synthesis of HBS-066-054: Compound HBS-066-048 (0.670 g, 1.82 mmol) was dissolved in anhydrous dioxane (20 mL). The 4.0 M HCl in dioxane (2.30 mL, 9.20 mmol) was added and the reaction was stirred vigorously at 60° C. for 72 h. LCMS showed complete conversion to product m/z 268. The reaction mixture was cooled to RT, filtered, and washed with Hexane to obtain a crusty solid that adhered to the filter paper. The sample was dissolved in MeOH, concentrated, and dried in a vacuum oven to afford 0.53 g of off white crusty solid (Yield=85.9%). MS (ESI) mass calcd. for C₁₂H₁₄ClN₃S, 267.1; m/z found 268.1 [M+H]⁺.

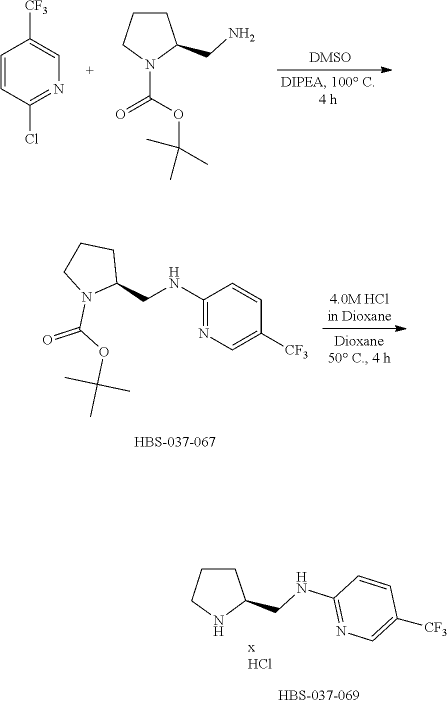 [SMILES: FC(F)(F)c1ccc(Cl)nc1 ; CC(C)(C)OC(=O)N1CCC[C@H]1CN ; CC(C)(C)OC(=O)N1CCC[C@H]1CNc1ccc(C(F)(F)F)cn1 ; FC(F)(F)c1ccc(NC[C@@H]2CCCN2)nc1]

Step 1: Synthesis of HBS-037-067: 2-Chloro-5-trifluoromethyl-pyridine (0.33 g, 1.82 mmol) and (S)-1-Boc-2-(aminomethyl)-pyrrolidine (0.36 g, 1.81 mmol) were dissolved in Dry DMSO (5.0 mL). The DIPEA (1.6 mL, 9.1 mmol) was added and rxn mixture was stirred at 100° C. temperature for 4 h. TLC shows product formation. The rxn mixture was diluted with water. The product was extracted with Ethyl acetate. The combined ethyl acetate layer was washed with water followed by brine. The organic layer was separated and dried over anhydrous sodium sulfate. The evaporation of solvent gave crude product. The crude product was purified by ISCO combi-flash chromatography system, Mobile phase: EtOAc:Hexane gradient. The desired liquid product was isolated 0.24 g (Yield 39.0%). MS (ESI) mass calcd. for C₁₆H₂₂F₃N₃O₂, 345.0; m/z found 346.1 [M+H]⁺.

Step 2: Synthesis of HBS-037-069: HBS-037-067 (0.24 g, 0.71 mmol) were dissolved in Dry Dioxane (3.0 mL). The 4.0 M HCl solution in dioxane (1.77 mL, 7.08 mmol) was added and rxn mixture was stirred at 50° C. temperature for 4 h. LCMS shows product formation m/z 246. The rxn mixture was concentrated under reduced pressure to obtain solid product (0.19 g, Yield 78.4%). MS (ESI) mass calcd. for C₁₁H₁₄F₃N₃, 245.2; m/z found 246.0 [M+H]⁺, ¹H NMR (400 MHz, CHLOROFORM-d) δ ppm 1.76-2.01 (m, 1H) 2.01-2.15 (m, 1H) 2.15-2.28 (m, 1H) 2.36 (br s, 1H) 3.25-3.46 (br s, 1H) 3.48 (br s, 1H) 4.04 (br s, 2H) 4.31 (br s, 1H) 7.47 (br s, 1H) 7.86 (br s, 1H) 8.19 (br s, 1H) 9.41-10.42 (br s, 1H).

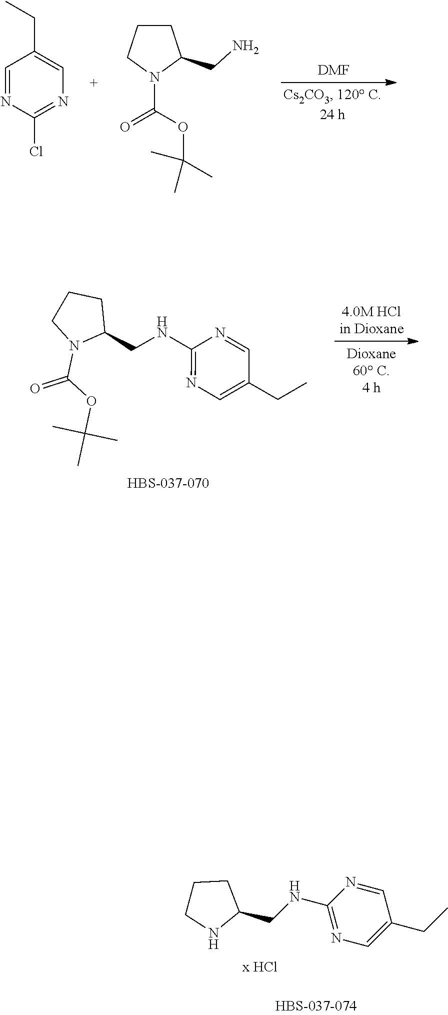

Step 1: Synthesis of HBS-037-070: 2-Chloro-5-ethyl-pyrimidine (0.2 g, 1.37 mmol) and (S)-1-Boc-2-(aminomethyl)-pyrrolidine (0.28 g, 1.37 mmol) were dissolved in Dry DMF (5.0 mL). The Cs₂CO₃ (0.89 g, 2.75 mmol) was added and rxn mixture was stirred at 120° C. temperature for 24 h. TLC shows product formation. The rxn mixture was diluted with water. The product was extracted with Ethyl acetate. The combined ethyl acetate layer was washed with water followed by brine. The organic layer was separated and dried over anhydrous sodium sulfate. The evaporation of solvent gave crude product. The crude product was purified by ISCO combi-flash chromatography system, Mobile phase: EtOAc:Hexane gradient. The desired liquid product was isolated 0.27 g (Yield 64.2%). MS (ESI) mass calcd. for C₁₆H₂₆N₄O₂, 306.4; m/z found 307.1 [M+H]⁺.

Step 2: Synthesis of HBS-037-074: HBS-037-070 (0.27 g, 0.88 mmol) were dissolved in Dry Dioxane (3.0 mL). The 4.0 M HCl solution in dioxane (2.2 mL, 8.81 mmol) was added and rxn mixture was stirred at 60° C. temperature for 4 h. LCMS shows product formation m/z 207. The rxn mixture was concentrated under reduced pressure to obtain solid product (0.31 g, Yield Quant.). MS (ESI) mass calcd. for C₁H₁₈N₄, 206.3; m/z found 207.1 [M+H]⁺.

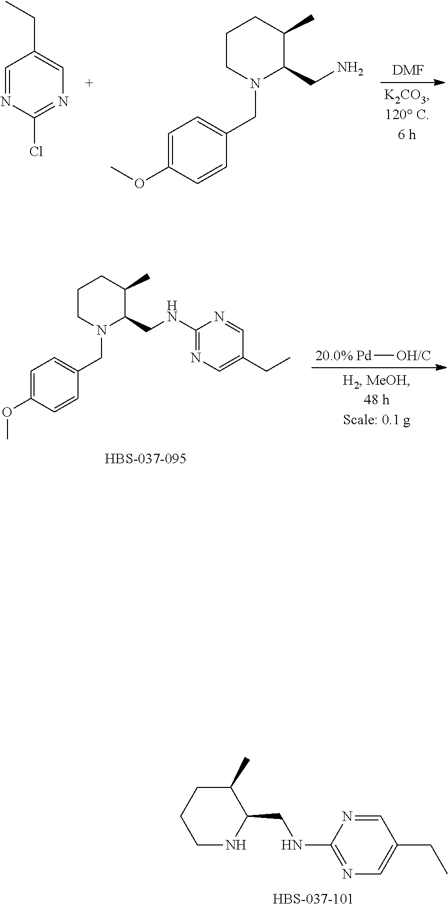

Step 1: Synthesis of HBS-037-095: [(2S,3R)-1-[4-methoxyphenyl)methyl]-3-methylpiperidine-2-yl]methanamine (0.35 g, 1.4 mmol) and 2-chloro-5-ethyl-pyrimidine (0.2 g, 1.4 mmol) were dissolved in Dry DMF (4.0 mL). The K₂CO₃ (0.39 g, 2.82 mmol) was added and rxn mixture was stirred at 120° C. temperature for 6 h. TLC shows product formation. The rxn mixture was diluted with water. The product was extracted with Ethyl acetate. The combined ethyl acetate layer was washed with water followed by brine. The organic layer was separated and dried over anhydrous sodium sulfate. The evaporation of solvent gave crude product. The crude product was purified by Combiflash chromatography system, Mobile phase: EtOAc:Hexane, gradient. The 0.33 g of product was obtained (Yield 65.4%). MS (ESI) mass calcd. for C₂₁H₃₀N₄O, 354.5; m/z found 355.2 [M+H]⁺, ¹H NMR (400 MHz, CHLOROFORM-d) δ ppm 0.85-0.94 (d, J=8.0 Hz, 3H) 1.16 (t, J=8.0 Hz, 3H) 1.25-1.47 (m, 2H) 1.50-1.84 (m, 2H) 2.05-2.25 (m, 1H) 2.35-2.47 (q, J=8.0 Hz, 2H) 2.47-2.60 (m, 1H) 2.62-2.83 (m, 2H) 3.33-3.46 (m, 2H) 3.77 (s, 3H) 3.78-3.85 (m, 2H) 5.68 (br s, 1H) 6.84 (d, J=8.66 Hz, 2H) 7.24-7.31 (m, 2H) 8.12 (s, 2H).

Step 2: Synthesis of HBS-037-101: HBS-037-095 (0.1 g, 0.3 mmol) was dissolved in MeOH (3.0 mL). The 20.0% Pd—OH/C (30.0 mg) was added and rxn mixture was stirred at ambient temperature for 24 h. The TLC shows little amount of starting material and product formation. The 20.0% Pd—OH/C (30.0 mg) was further added and rxn mixture was stirred at ambient temperature for another 24 h. The TLC shows completion of rxn. The LCMS data shows m/z 235 of product formation. The rxn mixture was filtered over celite and washed with MeOH. The filtrate was evaporated under reduced pressure to obtain 66.0 mg of crude product. The crude product is used in next step without purification. MS (ESI) mass calcd. for C₁₃H₂₂N₄, 234.3; m/z found 235.2 [M+H]⁺.

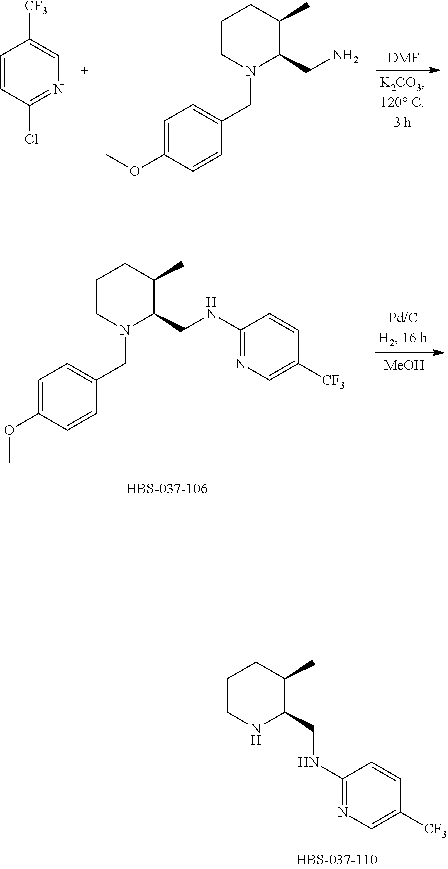

Step 1: Synthesis of HBS-037-106: [(2S,3R)-1-[4-methoxyphenyl)methyl]-3-methylpiperidine-2-yl]methanamine (0.32 g, 1.29 mmol) and 2-chloro-5-trifluoromethyl-pyridine (0.23 g, 1.29 mmol) were dissolved in Dry DMF (5.0 mL). The K₂CO₃ (0.36 g, 2.58 mmol) was added and rxn mixture was stirred at 120° C. temperature for 4 h. TLC shows product formation and LCMS shows m/z 394 of product formation. The rxn mixture was diluted with water. The product was extracted with Ethyl acetate. The combined ethyl acetate layer was washed with water followed by brine. The organic layer was separated and dried over anhydrous sodium sulfate. The evaporation of solvent gave crude product. The crude product was purified by ISCO combiflash chromatography system, Mobile phase: DCM:MeOH (90:10 v/v mL). The product band was isolated. The 0.41 g of pure product was obtained with m/z 394 (Yield 81.4%). MS (ESI) mass calcd. For C₂₁H₂₆F₃N₃O, 393.5; m/z found 394.1 [M+H]⁺, ¹H NMR (400 MHz, CHLOROFORM-d) δ ppm 0.88 (d, J=7.04 Hz, 3H) 1.19-1.46 (m, 2H) 1.54-1.65 (m, 1H) 1.65-1.83 (m, 1H) 2.07-2.22 (m, 1H) 2.50-2.65 (m, 1H) 2.65-2.80 (m, 2H) 3.20-3.38 (m, 2H) 3.78 (s, 3H) 3.79-3.84 (m, 2H) 5.67 (br s, 1H) 6.32 (d, J=8.80 Hz, 1H) 6.80-6.89 (m, 2H) 7.15-7.29 (m, 2H) 7.49 (dd, J=8.80, 2.35 Hz, 1H) 8.28-8.32 (m, 1H)

Step 2: Synthesis of HBS-037-110: HBS-037-106 (0.02 g, 0.5 mmol) was dissolved in MeOH (5.0 mL). The 10.0% Pd/C (60.0 mg) was added and rxn mixture was stirred at ambient temperature for 24 h under H₂ atm. The TLC shows completion of rxn. The LCMS data shows m/z 274 of product formation. The rxn mixture was filtered over celite and washed with MeOH. The filtrate was evaporated under reduced pressure to obtain 0.16 g of crude product. MS (ESI) mass calcd. for C₁₃H₁₈F₃N₃, 273.3; m/z found 274.1 [M+H]⁺.

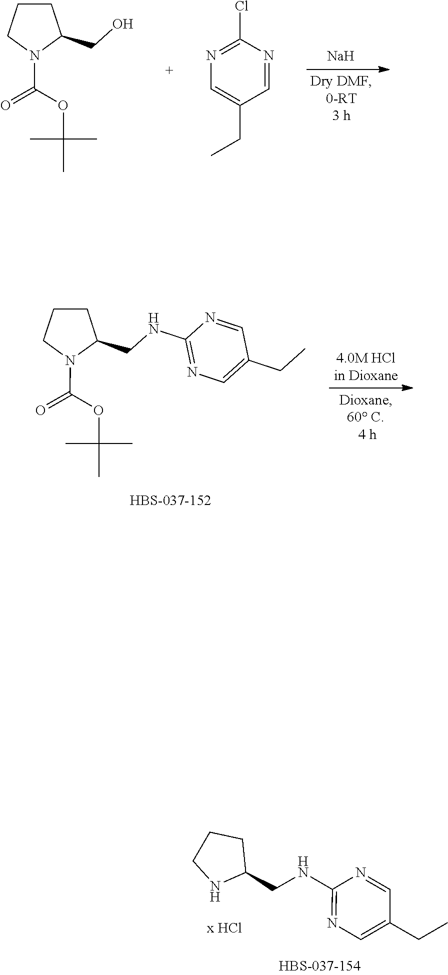

Step 1: Synthesis of HBS-037-152: N-Boc-L-Prolinol (0.2 g, 0.99 mmol) was dissolved in Dry DMF (4.0 mL). The NaH (0.08 g, 2.0 mmol) was added under ice cooling. The 2-Chloro-5-Ethyl-Pyrimidine (0.2 g, 1.5 mmol) was added under cooling and rxn mixture was gradually warmed at room temperature under stirring for 3 h. The LCMS shows product formation m/z 308.2. The rxn mixture was diluted with water. The product was extracted with Ethyl acetate three times. The EtOAc layers were separated and dried over anhydrous Na₂SO₄. The evaporation of solvent gave crude product. The crude product was purified by column chromatography, Mobile phase: EtOAc:Hexane gradient. The 0.3 g of pure product was obtained (Yield Quantitative). MS (ESI) mass calcd. for C₁₆H₂₆N₃O₃, 307.4; m/z found 308.2 [M+H]⁺.

Step 2: Synthesis of HBS-037-154: HBS-037-152 (0.3 g, 0.99 mmol) were dissolved in Dry Dioxane (4.0 mL). The 4.0 M HCl solution in dioxane (2.48 mL, 9.9 mmol) was added and rxn mixture was stirred at 60° C. temperature for 4 h. LCMS shows product formation m/z 208.1. The rxn mixture was concentrated under reduced pressure to obtain 0.32 g of liquid product. MS (ESI) mass calcd. for C₁₁H₁₇N₃O, 207.3; m/z found 208.1 [M+H]⁺.

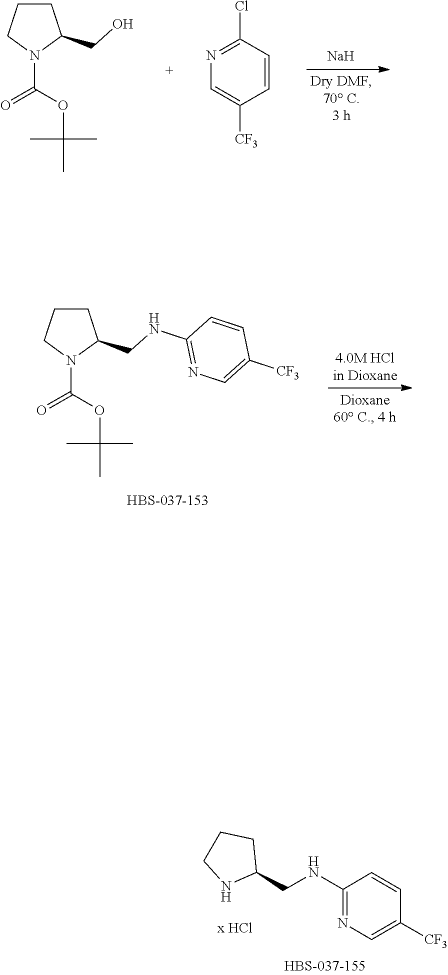

Step 1: Synthesis of HBS-037-153: N-Boc-L-Prolinol (0.2 g, 0.99 mmol) was dissolved in Dry DMF (4.0 mL). The NaH (0.08 g, 2.0 mmol) was added followed by 2-Chloro-5-trifluoromethylpyridine (0.27 g, 1.5 mmol). The rxn mixture was heated at 70° C. temperature for 3 h. The LCMS shows product formation m/z 347.1. The rxn mixture was diluted with water. The product was extracted with Ethyl acetate three times. The EtOAc layers were separated and dried over anhydrous Na₂SO₄. The evaporation of solvent gave crude product. The crude product was purified by column chromatography, Mobile phase: EtOAc: Hexane gradient. The 0.3 g of pure product was obtained (Yield 86.0%). MS (ESI) mass calcd. for C₁₆H₂₁F₃N₂O₃, 346.3; m/z found 347.1 [M+H]⁺.

Step 1: Synthesis of HBS-037-155: HBS-037-153 (0.3 g, 0.86 mmol) were dissolved in Dry Dioxane (2.0 mL). The 4.0 M HCl solution in dioxane (2.14 mL, 8.6 mmol) was added and rxn mixture was stirred at 60° C. temperature for 4 h. LCMS shows product formation m/z 247.1. The rxn mixture was filtered and washed with Hexane (5.0 mL×3) to obtain the 0.27 g of solid product (Yield Quant.). MS (ESI) mass calcd. for C₁₁H₁₃F₃N₂O, 246.2; m/z found 247.1 [M+H]⁺, ¹H NMR (400 MHz, CHLOROFORM-d) δ ppm 1.87-1.99 (m, 1H) 1.99-2.08 (m, 1H) 2.08-2.17 (m, 1H) 2.17-2.29 (m, 1H) 3.32-3.49 (m, 2H) 3.96-4.09 (m, 1H) 4.60-4.77 (m, 2H) 7.00 (d, J=8.73 Hz, 1H) 7.77 (dd, J=8.73, 2.35 Hz, 1H) 8.37-8.42 (m, 1H) 9.74 (br s, 1H) 10.32 (br s, 1H).

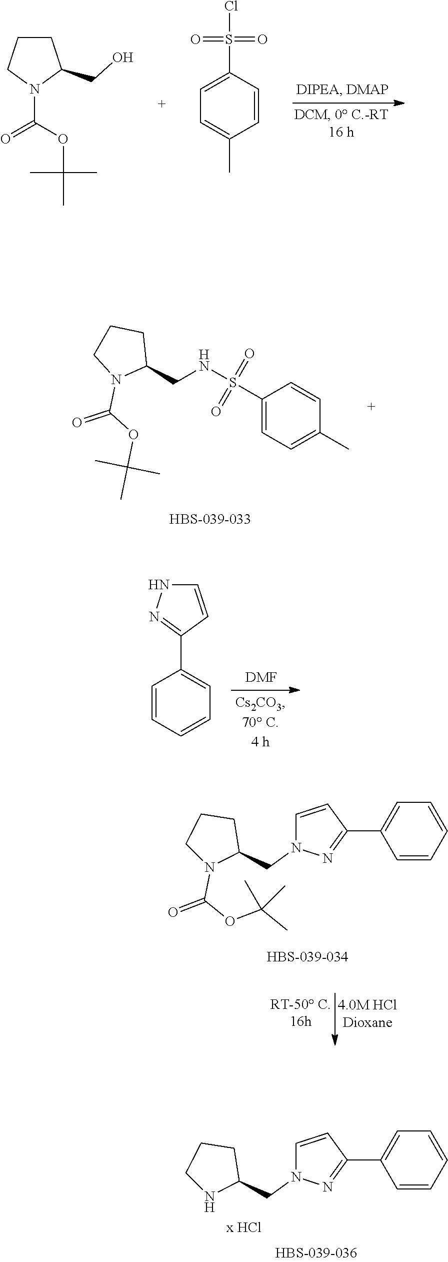

Step 1: Synthesis of HBS-039-033: The N-Boc-L-Prolinol (0.5 g, 2.48 mmol) was dissolved in DCM (10.0 mL). The DIPEA (0.9 mL, 4.97 mmol) was added followed by DMAP (0.61 g, 4.97 mmol). The rxn mixture was cooled in ice bath and p-TsCl (0.52 g, 2.73 mmol) was added. The rxn mixture was stirred and gradually warmed to room temperature for 16 h. The LCMS shows product formation m/z 256, 300. The rxn mixture was diluted with water. The product was extracted with DCM. The DCM layer was separated and dried over anhydrous Na2SO₄. The evaporation of solvent gave 0.88 g of product (Yield Quant.). MS (ESI) mass calcd. for C₁₇H₂₅NO₅S, 355.5; m/z found 300.1, 256.1 [M+H]⁺.

Step 2: Synthesis of HBS-039-034: HBS-039-033 (0.88 g, 2.48 mmol) and 3-Phenyl-1H-Pyrazole (0.43 g, 2.98 mmol) were dissolved in Dry DMF (5.0 mL). The Cs₂CO₃ (1.61 g, 4.96 mmol) was added and the rxn mixture was stirred at 70° C. temperature for 4 h. The LCMS shows product formation m/z 328. The rxn mixture was cooled at ambient temperature and diluted with water. The product was extracted with Ethyl acetate. The EtOAc layer was separated and dried over anhydrous Na₂SO₄. The evaporation of solvent gave crude product. The crude product was purified by column chromatography, Mobile phase: EtOAc:Hexane gradient. The 0.73 g of pure product was obtained (Yield 89.5%). MS (ESI) mass calcd. for C₁₉H₂₅N₃O₂, 327.4; m/z found 328.3 [M+H]⁺.

Step 3: Synthesis of HBS-039-036: HBS-039-034 (0.73 g, 2.24 mmol) was dissolved in Dry Dioxane (10.0 mL). The 4.0 M HCl solution in dioxane (2.8 mL, 11.2 mmol) was added and rxn mixture was stirred at 50° C. temperature for 16 h. LCMS shows product formation m/z 228. The rxn mixture was filtered and washed with Hexane (5.0 mL×3) to obtain the 0.53 g of solid product (Yield Quant.). MS (ESI) mass calcd. for C₁₄H₁₇N₃, 227.1; m/z found 228.2 [M+H]⁺, ¹H NMR (400 MHz, CHLOROFORM-d) δ ppm 1.74-1.94 (m, 1H) 1.94-2.05 (m, 1H) 2.06-2.15 (m, 1H) 2.16-2.35 (m, 1H) 3.21-3.45 (m, 2H) 4.33 (br s, 1H) 4.86 (dd, J=14.82, 3.96 Hz, 1H) 5.07 (br dd, J=14.67, 8.66 Hz, 1H) 6.75 (d, J=2.20 Hz, 1H) 7.34-7.48 (m, 3H) 7.76-7.87 (m, 2H) 8.44 (d, J=2.49 Hz, 1H) 9.51-10.12 (br, 1H).

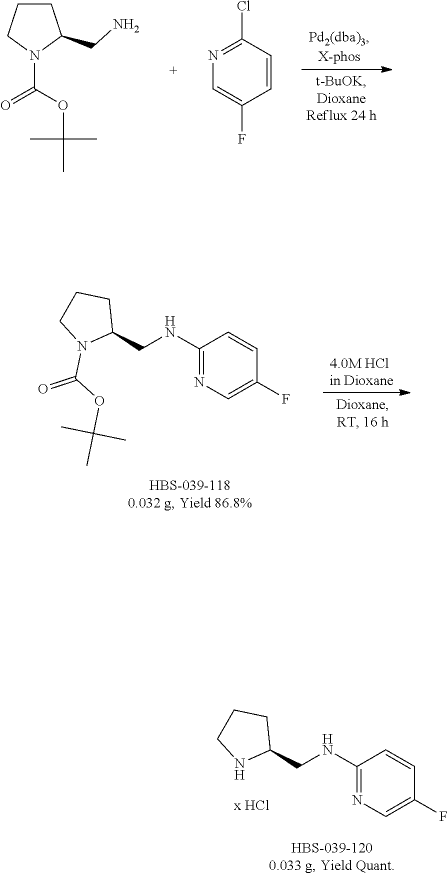 [SMILES: CC(C)(C)OC(=O)N1CCC[C@H]1CN ; Fc1ccc(Cl)nc1 ; "Pd2(dba)3,X-phos" ; CC(C)(C)OC(=O)N1CCC[C@H]1CNc1ccc(F)cn1 ; Fc1ccc(NC[C@@H]2CCCN2)nc1]

Step 1: Synthesis of HBS-039-118: (S)-1-Boc-2-(amino methyl)pyrrolidine (1.5 g, 5.6 mmol) and 2-Chloro-5-fluoro pyridine (0.033 g, 0.25 mmol) were dissolved in Dioxane (3.0 mL). The anhydrous t-BuOK (0.042 g, 0.37 mmol) was added followed by Pd₂(dba)₃ (0.023 g, 0.025 mmol) and X-Phos (0.012 g, 0.025 mmol). The rxn mixture was stirred at 110° C. temperature under N₂ atm. for 4 h. The LCMS data shows product formation m/z 296.0. The rxn mixture was filtered over celite bed and washed with ethyl acetate. The filtrate was concentrated under reduced pressure to obtain the crude product. The crude product was purified by ISCO combi-flash chromatography system, Mobile phase: EtOAc:Hexane gradient. The 0.032 g of product was obtained (Yield 86.8%). MS (ESI) mass calcd. for C₁₅H₂₂FN₃O₂, 295.35; m/z found 296.0 [M+H]⁺.

Step 2: Synthesis of HBS-039-120: Compound HBS-039-118 (0.032 g, 0.11 mmol) was dissolved in Dry Dioxane (1.0 mL). The 4.0 M HCl solution in dioxane (0.27 mL, 1.1 mmol) was added and rxn mixture was stirred at ambient temperature for 16 h. LCMS shows product formation m/z 196.1. The rxn mixture was concentrated under reduced pressure to obtain the 0.033 g of product (Yield Quant.). MS (ESI) mass calcd. for C₁₀H₁₄FN₃, 195.24; m/z found 196.1 [M+H]⁺.

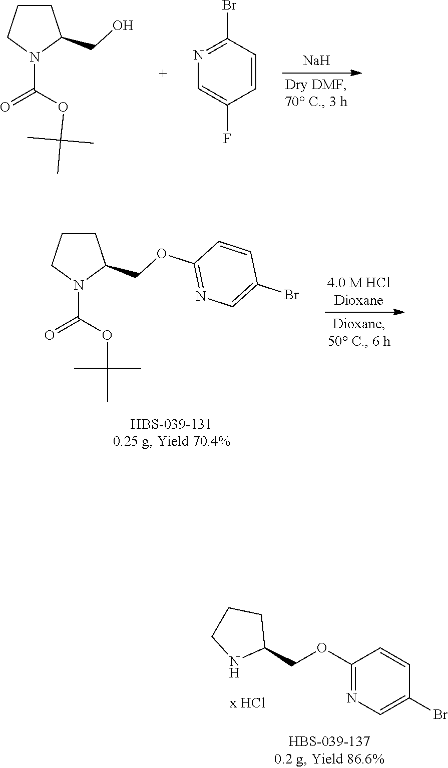

Step 1: Synthesis of HBS-039-131: N-Boc-L-prolinol (0.2 g, 0.99 mmol) was dissolved in Dry DMF (2.0 mL). The NaH (0.12 g, 2.98 mmol) was added at 0° C. temperature. The rxn mixture was stirred at 0° C. temperature for 30.0 min. The 2-Bromo-5-fluoro-pyridine (0.26 g, 1.49 mmol) solution in DMF (1.0 mL) was added at 0° C. temperature. The rxn mixture was gradually warmed at ambient temperature and heated at 70° C. temperature for 3 h. The LCMS shows product formation m/z 357.1. The rxn mixture was cooled at ambient temperature and diluted with water. The product was extracted with Ethyl acetate. The EtOAc layer was separated and dried over anhydrous Na₂SO₄. The evaporation of solvent gave crude product. The crude product was purified by column chromatography, Mobile phase: EtOAc:Hexane gradient. The 0.25 g of product was obtained (Yield 70.4%). MS (ESI) mass calcd. for C₁₅H₂₁BrN₂O₃, 357.24; m/z found 357.1 [M+H]⁺.

Step 2: Synthesis of HBS-039-137: Compound HBS-039-131 (0.25 g, 0.7 mmol) was dissolved in Dry Dioxane (5.0 mL). The 4.0 M HCl solution in dioxane (1.74 mL, 7.0 mmol) was added and rxn mixture was stirred at 50° C. temperature for 6 h. LCMS shows product formation m/z 257. The rxn mixture was filtered and dried to obtain the 0.2 g of product (Yield 86.6%). MS (ESI) mass calcd. for C₁₀H₁₃BrN₂O, 257.13; m/z found 257.0 [M+H]⁺.

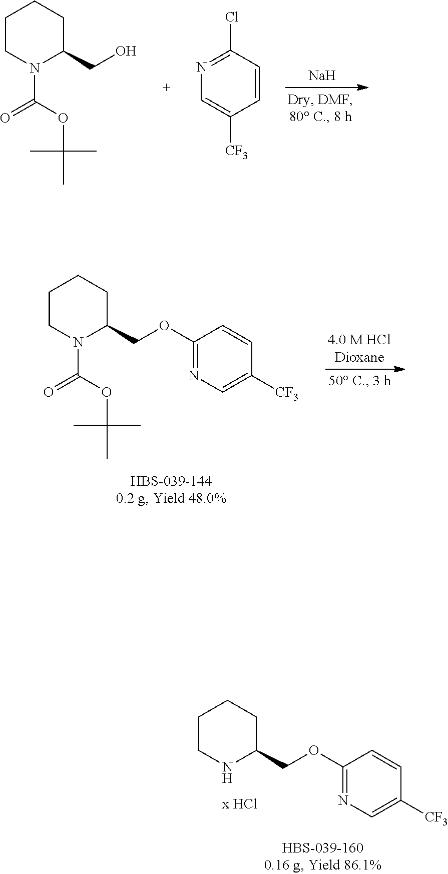

Step 1: Synthesis of HBS-039-144: (S)-1-Boc-2-(hydroxymethyl)piperidine (0.25 g, 1.16 mmol) was dissolved in anhydrous DMF (5.0 mL). The NaH (0.14 g, 3.48 mmol) was added at 0° C. temperature and reaction mixture was stirred for 15.0 min. The 2-chloro-5-trifluoromethylpyridine (0.32 g, 1.74 mmol) was added and the reaction mixture was gradually warmed at ambient temperature. The reaction mixture was heated at 80° C. for 8 h. LCMS shows product formation m/z 361.2. The rxn mixture was cooled at ambient temperature and diluted with water. The product was extracted with Ethyl acetate. The EtOAc layer was separated and dried over anhydrous Na₂SO₄. The evaporation of solvent gave crude product. The crude product was purified by column chromatography, Mobile phase: EtOAc:Hexane gradient. The 0.2 g of product was obtained (Yield 48.0%). MS (ESI) mass calcd. for C₁₇H₂₃F₃N₂O₃, 360.37; m/z found 361.2 [M+H]⁺.

Step 2: Synthesis of HBS-039-160: Compound HBS-039-144 (0.2 g, 0.56 mmol) was dissolved in anhydrous Dioxane (5.0 mL). The 4.0 M HCl in Dioxane (1.39 mL, 5.58 mmol) was added and the reaction was heated at 50° C. temperature for 3 h. LCMS shows product formation m/z 261.1. The reaction mixture was concentrated under reduced pressure to obtain 0.16 g of product (Yield 86.1%). MS (ESI) mass calcd. for C₁₂H₁₅F₃N₂O, 260.26; m/z found 261.1 [M+H]⁺.

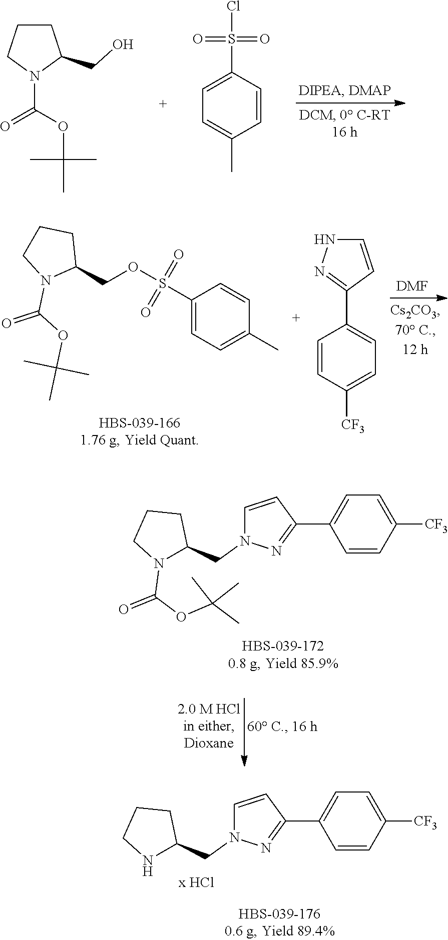

Step 1: Synthesis of HBS-039-166: The N-Boc-L-Prolinol (1.0 g, 4.97 mmol) was dissolved in DCM (15.0 mL). The DIPEA (1.72 mL, 9.94 mmol) was added followed by DMAP (1.21 g, 9.94 mmol). The rxn mixture was cooled at 0° C. in Ice bath. The p-TsCl (1.04 g, 5.46 mmol) was added. The rxn mixture was stirred and gradually warmed to room temperature for 16 h. The LCMS shows product formation m/z 256. The reaction mixture was diluted with water. The product was extracted with DCM. The DCM layer was separated and dried over anhydrous Na₂SO₄. The evaporation of solvent gave 1.76 g of product (Yield Quant.). MS (ESI) mass calcd. for C₁₇H₂₅NO₅S, 355.45; m/z found, 256.0 [M−Boc]⁺.

Step 2: Synthesis of HBS-039-172: Compound HBS-039-166 (0.84 g, 2.36 mmol) and 3-[4-(trifluoromethyl)Phenyl]-1H-Pyrazole (0.5 g, 2.36 mmol) were dissolved in Dry DMF (10.0 mL). The anhydrous Cs₂CO₃ (1.53 g, 4.71 mmol) was added and the rxn mixture was stirred at 70° C. temperature for 12 h. The LCMS shows product formation m/z 396.3. The rxn mixture was cooled at ambient temperature and diluted with water. The product was extracted with Ethyl acetate. The combined EtOAc layer was separated and dried over anhydrous Na₂SO₄. The evaporation of solvent gave crude product. The crude product was purified by column chromatography, Mobile phase: EtOAc:Hexane gradient. The 0.8 g of pure product was obtained (Yield 85.9%). MS (ESI) mass calcd. for C₂₀H₂₄F₃N₃O₂, 395.42; m/z found 396.3 [M+H]⁺.

Step 3: Synthesis of HBS-039-176: Compound HBS-039-172 (0.8 g, 2.02 mmol) was dissolved in Dry Dioxane (20.0 mL). The 2.0 M HCl solution in Diethyl ether (4.1 mL, 8.1 mmol) was added and rxn mixture was stirred at 60° C. temperature for 16 h. LCMS shows product formation m/z 296.1. The rxn mixture cooled and ppts were filtered. The ppts were dried to obtain the 0.6 g of solid product (Yield 89.4%). MS (ESI) mass calcd. for C₁₄H₁₇N3, 227.1; m/z found 228.2 [M+H]⁺.

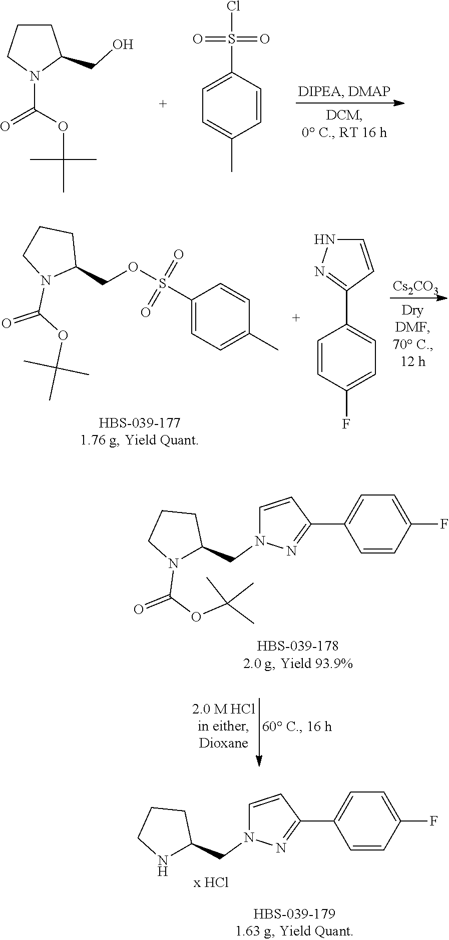

Step 1: Synthesis of HBS-039-177: The N-Boc-L-Prolinol (1.0 g, 4.97 mmol) was dissolved in DCM (15.0 mL). The DIPEA (1.72 mL, 9.94 mmol) was added followed by DMAP (1.21 g, 9.94 mmol). The rxn mixture was cooled at 0° C. in Ice bath. The p-TsCl (1.04 g, 5.46 mmol) was added. The rxn mixture was stirred and gradually warmed to room temperature for 16 h. The LCMS shows product formation m/z 256.1, 300.1. The reaction mixture was diluted with water. The product was extracted with DCM. The DCM layer was separated and dried over anhydrous Na₂SO₄. The evaporation of solvent gave 1.76 g of product (Yield Quant.). MS (ESI) mass calcd. for C₁₇H₂₅NO₅S, 355.45; m/z found, 256.1, 300.1 [M−Boc]⁺.

Step 2: Synthesis of HBS-039-178: Compound HBS-039-177 (1.0 g, 6.17 mmol) and 3-[4-fluorophenyl]-1H-Pyrazole (1.0 g, 6.17 mmol) were dissolved in Dry DMF (20.0 mL). The anhydrous Cs₂CO₃ (4.0 g, 12.33 mmol) was added and the rxn mixture was stirred at 70° C. temperature for 12 h. The LCMS shows product formation m/z 346.3. The rxn mixture was cooled at ambient temperature and diluted with water. The product was extracted with Ethyl acetate. The combined EtOAc layer was separated and dried over anhydrous Na₂SO₄. The evaporation of solvent gave crude product. The crude product was purified by column chromatography, Mobile phase: EtOAc:Hexane gradient. The 2.0 g of pure product was obtained (Yield 93.9%). MS (ESI) mass calcd. for C₁₉H₂₄FN₃O₂, 345.41; m/z found 346.3 [M+H]⁺.

Step 3: Synthesis of HBS-039-179: Compound HBS-039-178 (2.0 g, 5.79 mmol) was dissolved in Dry Dioxane (20.0 mL). The 2.0 M HCl solution in Diethyl ether (11.58 mL, 23.16 mmol) was added and rxn mixture was stirred at 60° C. temperature for 16 h. LCMS shows product formation m/z 24621. The rxn mixture cooled and ppts were filtered. The ppts were dried to obtain 1.63 g of solid product (Yield Quant.). MS (ESI) mass calcd. for C₁₄H₁₆FN₃, 245.3; m/z found 246.2 [M+H]⁺.

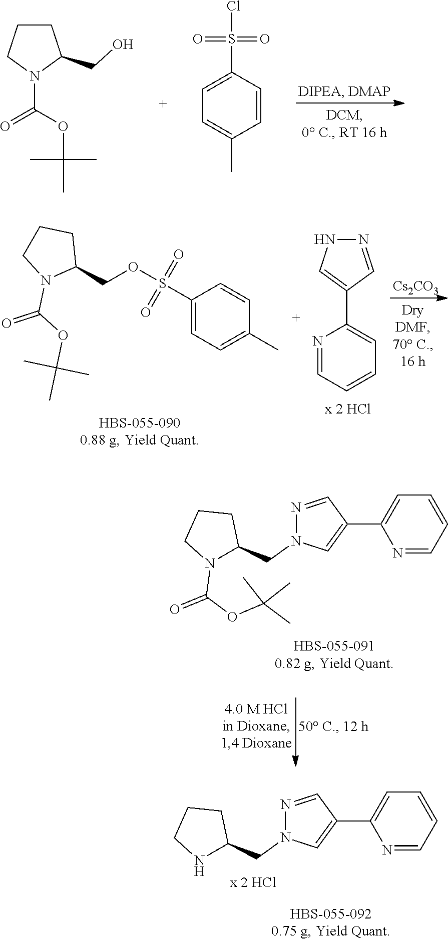

Step 1: Synthesis of HBS-055-090: The N-Boc-L-Prolinol (0.5 g, 2.48 mmol) was dissolved in DCM (10.0 mL). The DIPEA (0.9 mL, 4.97 mmol) was added followed by DMAP (0.61 g, 4.97 mmol). The rxn mixture was cooled at 0° C. in Ice bath. The p-TsCl (0.52 g, 2.73 mmol) was added. The rxn mixture was stirred and gradually warmed to room temperature for 16 h. The LCMS shows product formation m/z 256.1, 300.1. The reaction mixture was diluted with water. The product was extracted with DCM. The DCM layer was separated and dried over anhydrous Na₂SO₄. The evaporation of solvent gave 0.88 g of product (Yield Quant.). MS (ESI) mass calcd. for C₁₇H₂₅NO₅S, 355.45; m/z found, 256.1, 300.1 [M−Boc]⁺.

Step 2: Synthesis of HBS-055-091: Compound HBS-055-090 (0.88 g, 2.48 mmol) and 2-(1H-pyrazol-4-yl)pyridine dihydrochloride (0.6 g, 2.73 mmol) were dissolved in Dry DMF (10.0 mL). The anhydrous Cs₂CO₃ (1.61 g, 4.97 mmol) was added and the rxn mixture was stirred at 70° C. temperature for 16 h. The LCMS shows product formation m/z 329.2. The rxn mixture was cooled at ambient temperature and diluted with water. The product was extracted with Ethyl acetate. The combined EtOAc layer was separated and dried over anhydrous Na₂SO₄. The evaporation of solvent gave crude product. The crude product was purified by column chromatography, Mobile phase: EtOAc:Hexane gradient. The 0.82 g of pure product was obtained (Yield 81.5%). MS (ESI) mass calcd. for C₁₈H₂₄N₄O₂, 328.41; m/z found 329.2 [M+H]⁺.

Step 3: Synthesis of HBS-055-092: Compound HBS-055-091 (0.82 g, 2.48 mmol) was dissolved in Dry Dioxane (10.0 mL). The 4.0 M HCl solution in Dioxane (2.5 mL, 9.94 mmol) was added and rxn mixture was stirred at 50° C. temperature for 12 h. LCMS shows product formation m/z 229.2. The rxn mixture cooled and ppts were filtered. The ppts were dried to obtain 0.75 g of solid product (Yield Quant.). MS (ESI) mass calcd. for C₁₃H₁₆N₄, 228.29; m/z found 229.1 [M+H]⁺.

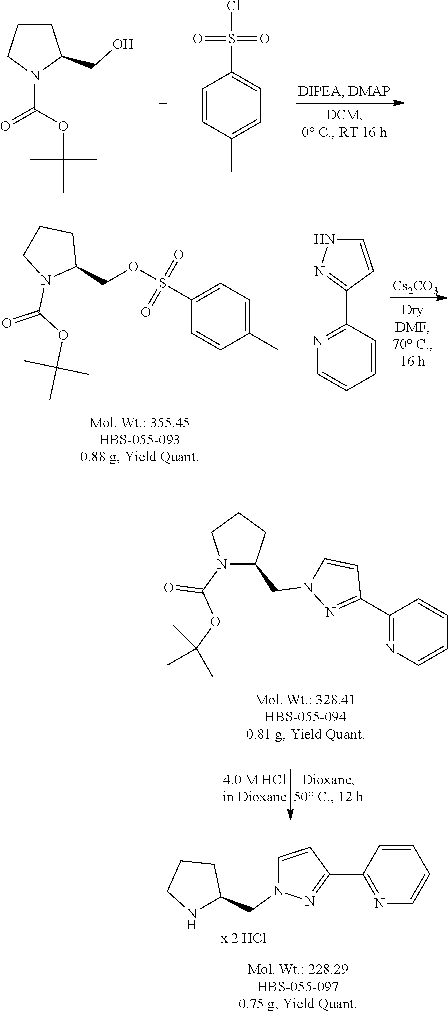

Step 1: Synthesis of HBS-055-093: The N-Boc-L-Prolinol (0.5 g, 2.48 mmol) was dissolved in DCM (10.0 mL). The DIPEA (0.9 mL, 4.97 mmol) was added followed by DMAP (0.61 g, 4.97 mmol). The rxn mixture was cooled at 0° C. in Ice bath. The p-TsCl (0.52 g, 2.73 mmol) was added. The rxn mixture was stirred and gradually warmed to room temperature for 16 h. The LCMS shows product formation m/z 256.1, 300.1. The reaction mixture was diluted with water. The product was extracted with DCM. The DCM layer was separated and dried over anhydrous Na₂SO₄. The evaporation of solvent gave 0.88 g of product (Yield Quant.). MS (ESI) mass calcd. for C₁₇H₂₅NO₅S, 355.45; m/z found, 256.1, 300.1 [M−Boc]⁺.

Step 2: Synthesis of HBS-055-094: Compound HBS-055-093 (0.88 g, 2.48 mmol) and 2-(1H-pyrazol-3-yl)pyridine (0.43 g, 2.98 mmol) were dissolved in Dry DMF (10.0 mL). The anhydrous Cs₂CO₃ (1.62 g, 4.97 mmol) was added and the rxn mixture was stirred at 70° C. temperature for 12 h. The LCMS shows product formation m/z 329.2. The rxn mixture was cooled at ambient temperature and diluted with water. The product was extracted with Ethyl acetate. The combined EtOAc layer was separated and dried over anhydrous Na₂SO₄. The evaporation of solvent gave crude product. The crude product was purified by column chromatography, Mobile phase: EtOAc:Hexane gradient. The 0.81 g of product was obtained (Yield Quant.). MS (ESI) mass calcd. for C₁₈H₂₄N₄O₂, 328.41; m/z found 329.2 [M+H]⁺.

Step 3: Synthesis of HBS-055-097: Compound HBS-055-094 (0.81 g, 2.48 mmol) was dissolved in Dry Dioxane (10.0 mL). The 4.0 M HCl solution in Dioxane (2.5 mL, 9.93 mmol) was added and rxn mixture was stirred at 50° C. temperature for 12 h. LCMS shows product formation m/z 229.2. The rxn mixture cooled and ppts were filtered. The ppts were dried to obtain 0.75 g of solid product (Yield Quant.). MS (ESI) mass calcd. for C₁₃H₁₆N₄, 228.29; m/z found 229.1 [M+H]⁺.

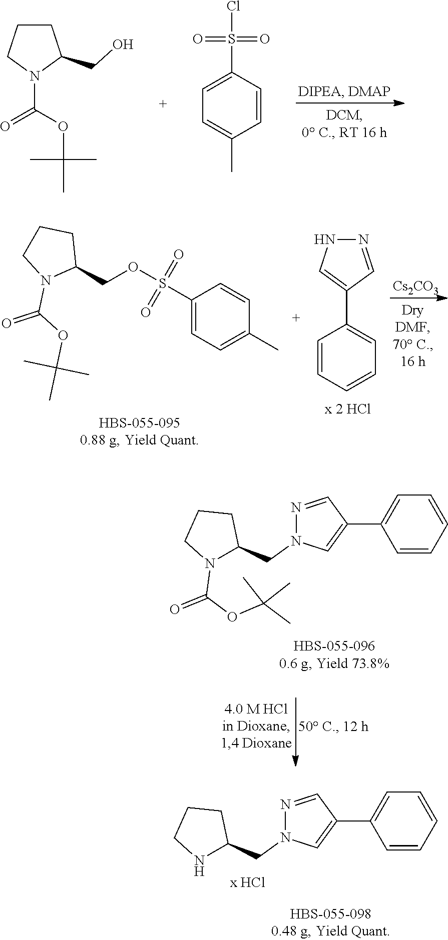

Step 1: Synthesis of HBS-055-095: The N-Boc-L-Prolinol (0.5 g, 2.48 mmol) was dissolved in DCM (10.0 mL). The DIPEA (0.9 mL, 4.97 mmol) was added followed by DMAP (0.61 g, 4.97 mmol). The rxn mixture was cooled at 0° C. in Ice bath. The p-TsCl (0.52 g, 2.73 mmol) was added. The rxn mixture was stirred and gradually warmed to room temperature for 16 h. The LCMS shows product formation m/z 256.1, 300.1. The reaction mixture was diluted with water. The product was extracted with DCM. The DCM layer was separated and dried over anhydrous Na₂SO₄. The evaporation of solvent gave 0.88 g of product (Yield Quant.). MS (ESI) mass calcd. for C₁₇H₂₅NO₅S, 355.45; m/z found, 256.1, 300.1 [M−Boc]⁺.

Step 2: Synthesis of HBS-055-096: Compound HBS-055-095 (0.88 g, 2.48 mmol) and 4-Phenyl-1H-pyrazole (0.72 g, 4.97 mmol) were dissolved in Dry DMF (10.0 mL). The anhydrous Cs₂CO₃ (1.62 g, 4.97 mmol) was added and the rxn mixture was stirred at 70° C. temperature for 16 h. The LCMS shows product formation m/z 328.2. The rxn mixture was cooled at ambient temperature and diluted with water. The product was extracted with Ethyl acetate. The combined EtOAc layer was separated and dried over anhydrous Na₂SO₄. The evaporation of solvent gave crude product. The crude product was purified by column chromatography, Mobile phase: EtOAc:Hexane gradient. The 0.6 g of pure product was obtained (Yield 73.8%). MS (ESI) mass calcd. for C₁₉H₂₅N₃O₂, 327.42; m/z found 328.2 [M+H]⁺.

Step 3: Synthesis of HBS-055-098: Compound HBS-055-096 (0.6 g, 1.83 mmol) was dissolved in Dry Dioxane (10.0 mL). The 4.0 M HCl solution in Dioxane (1.83 mL, 7.33 mmol) was added and rxn mixture was stirred at 50° C. temperature for 12 h. LCMS shows product formation m/z 228.2. The rxn mixture cooled and ppts were filtered. The ppts were dried to obtain 0.48 g of solid product (Yield Quant.). MS (ESI) mass calcd. for C₁₄H₁₇N₃, 227.31; m/z found 228.2 [M+H]⁺.

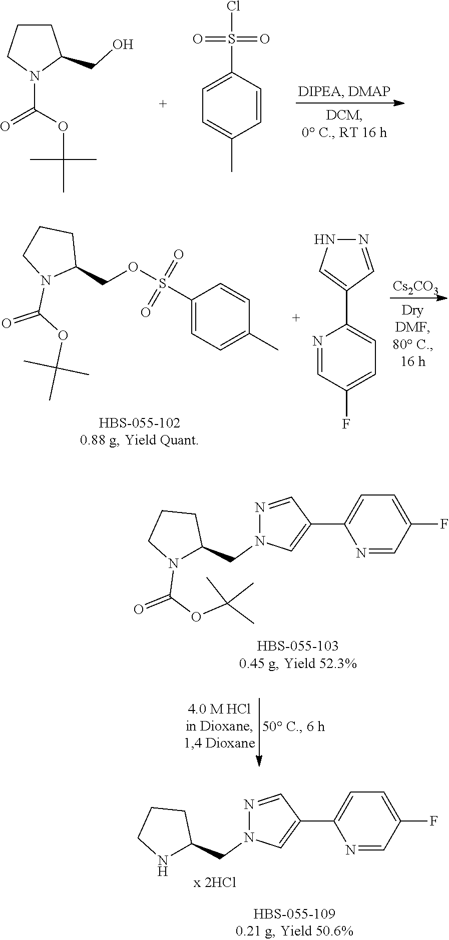

Step 1: Synthesis of HBS-055-102: The N-Boc-L-Prolinol (0.5 g, 2.48 mmol) was dissolved in DCM (10.0 mL). The DIPEA (0.9 mL, 4.97 mmol) was added followed by DMAP (0.61 g, 4.97 mmol). The rxn mixture was cooled at 0° C. in the Ice bath. The p-TsCl (0.52 g, 2.73 mmol) was added. The rxn mixture was stirred and gradually warmed to room temperature for 16 h. The LCMS shows product formation m/z 256.1, 300.1. The reaction mixture was diluted with water. The product was extracted with DCM. The DCM layer was separated and dried over anhydrous Na₂SO₄. The evaporation of solvent gave 0.88 g of product (Yield Quant.). MS (ESI) mass calcd. for C₁₇H₂₅NO₅S, 355.45; m/z found, 256.1, 300.1 [M−Boc]⁺.

Step 2: Synthesis of HBS-055-103: Compound HBS-055-102 (0.88 g, 2.48 mmol) and 5-Fluoro-2-(1H-pyrazole-4-yl)pyridine (0.7 g, 2.98 mmol) were dissolved in Dry DMF (10.0 mL). The anhydrous Cs₂CO₃ (2.64 g, 8.1 mmol) was added and the rxn mixture was stirred at 80° C. temperature for 16 h. The LCMS shows product formation m/z 347.2. The rxn mixture was cooled at ambient temperature and diluted with water. The product was extracted with Ethyl acetate. The combined EtOAc layer was separated and dried over anhydrous Na₂SO₄. The evaporation of solvent gave crude product. The crude product was purified by column chromatography, Mobile phase: EtOAc:Hexane gradient. The 0.45 g of pure product was obtained (Yield 52.3%). MS (ESI) mass calcd. for C₁₈H₂₃FN₄O₂, 346.4; m/z found 347.2 [M+H]⁺.

Step 3: Synthesis of HBS-055-109: Compound HBS-055-103 (0.45 g, 1.3 mmol) was dissolved in Dry Dioxane (10.0 mL). The 4.0 M HCl solution in Dioxane (1.3 mL, 5.2 mmol) was added and rxn mixture was stirred at 50° C. temperature for 6 h. LCMS shows product formation m/z 246.28. The rxn mixture cooled and ppts were filtered. The ppts were dried to obtain 0.21 g of solid product (Yield 50.6%). MS (ESI) mass calcd. for C₁₃H₁₅FN₄, 246.28; m/z found 247.1 [M+H]⁺.

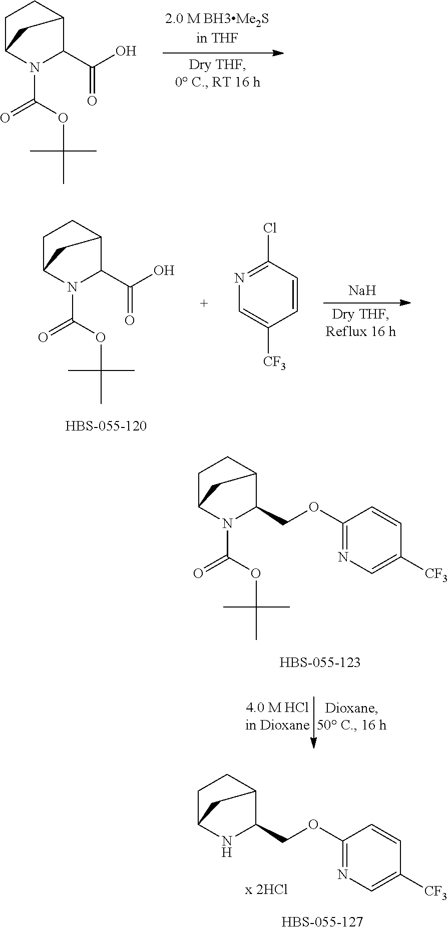

Step 1: Synthesis of HBS-055-120: (1R,3S,4S)-2-(Tert-butoxy carbonyl)-2-azabicyclo [2.2.1] Heptane-3-carboxylic acid (0.5 g, 2.1 mmol) was dissolved in Dry THF (10.0 mL). The 2.0 M BH₃·Me₂S (2.1 mL, 4.14 mmol) was added at 0° C. temperature. The rxn mixture gradually warmed at ambient temperature for 16 h. The LCMS shows product formation m/z 172.1, 250.1. The rxn mixture was quenched with Methanol. The reaction mixture was diluted with water. The product was extracted with Ethyl acetate. The EtOAc layers were separated and dried over anhydrous Na₂SO₄. The evaporation of solvent gave 0.47 g of crude product. (Yield Quant.). MS (ESI) mass calcd. for C₁₂H₂₁NO₃, 227.3; m/z found 172.1, 250.1 [M+Na]⁺.

Step 2: Synthesis of HBS-055-123: Compound HBS-055-120 (0.47 g, 2.1 mmol) was dissolved in Dry THF (10.0 mL). The NaH (0.166 g, 4.14 mmol) was added at 0° C. temperature. The 2-Chloro-5-trifluoromethylpyridine (0.45 g, 2.48 mmol) was added and reaction mixture was gradually heated at reflux temperature for 16 h. The LCMS shows product formation m/z 373.1. The rxn mixture was diluted with water. The product was extracted with Ethyl acetate. The combined EtOAc layers were separated and dried over anhydrous Na₂SO₄. The evaporation of solvent gave crude product. The crude product was purified by column chromatography, Mobile phase: EtOAc:Hexane gradient. The 0.5 g of pure product was obtained (Yield 64.9%). MS (ESI) mass calcd. for C₁₈H₂₃F₃N₂O₃, 372.38; m/z found 373.1 [M+H]⁺.

Step 3: Synthesis of HBS-055-127: Compound HBS-055-123 (0.5 g, 1.34 mmol) was dissolved in Dioxane (10.0 mL). The 4.0 M HCl solution in dioxane (1.34 mL, 5.37 mmol) was added and rxn mixture was stirred at 50° C. temperature for 16 h. LCMS shows product formation m/z 273.1. The rxn mixture was cooled and ppts were filtered. The ppts were dried to obtain 0.41 g of solid product (Yield 88.4%). MS (ESI) mass calcd. for C₁₃H₁₅F₃N₂O, 272.27; m/z found 273.1 [M+H]⁺.

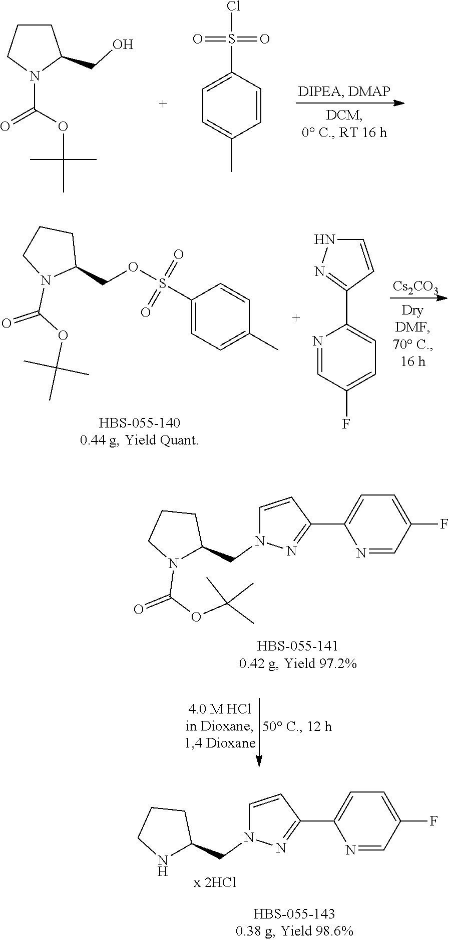

Step 1: Synthesis of HBS-055-140: The N-Boc-L-Prolinol (0.25 g, 1.24 mmol) was dissolved in DCM (5.0 mL). The DIPEA (0.43 mL, 2.48 mmol) was added followed by DMAP (0.3 g, 2.48 mmol). The rxn mixture was cooled at 0° C. in the Ice bath. The p-TsCl (0.26 g, 1.34 mmol) was added. The rxn mixture was stirred and gradually warmed to room temperature for 16 h. The LCMS shows product formation m/z 256.1, 300.1. The reaction mixture was diluted with water. The product was extracted with DCM. The DCM layer was separated and dried over anhydrous Na₂SO₄. The evaporation of solvent gave 0.44 g of product (Yield Quant.). MS (ESI) mass calcd. for C₁₇H₂₅NO₅S, 355.45; m/z found, 256.1, 300.1 [M−Boc]⁺.

Step 2: Synthesis of HBS-055-141: Compound HBS-055-140 (0.44 g, 1.24 mmol) and 5-Fluoro-2-(1H-pyrazole-3-yl)pyridine (0.24 g, 1.49 mmol) were dissolved in Dry DMF (8.0 mL). The anhydrous Cs₂CO₃ (1.21 g, 3.73 mmol) was added and the rxn mixture was stirred at 70° C. temperature for 16 h. The LCMS shows product formation m/z 347.2. The rxn mixture was cooled at ambient temperature and diluted with water. The product was extracted with Ethyl acetate. The combined EtOAc layer was separated and dried over anhydrous Na₂SO₄. The evaporation of solvent gave crude product. The crude product was purified by column chromatography, Mobile phase: EtOAc:Hexane gradient. The 0.42 g of pure product was obtained (Yield 97.2%). MS (ESI) mass calcd. for C₁₈H₂₃FN₄O₂, 346.4; m/z found 347.2 [M+H]⁺.

Step 3: Synthesis of HBS-055-143: Compound HBS-055-141 (0.42 g, 1.21 mmol) was dissolved in Dry Dioxane (10.0 mL). The 4.0 M HCl solution in Dioxane (1.21 mL, 4.83 mmol) was added and rxn mixture was stirred at 50° C. temperature for 12 h. LCMS shows product formation m/z 247.1. The rxn mixture was cooled and ppts were filtered. The ppts were dried to obtain 0.38 g of solid product (Yield 98.6%). MS (ESI) mass calcd. for C₁₃H₁₅FN₄, 246.28; m/z found 247.1 [M+H]⁺.

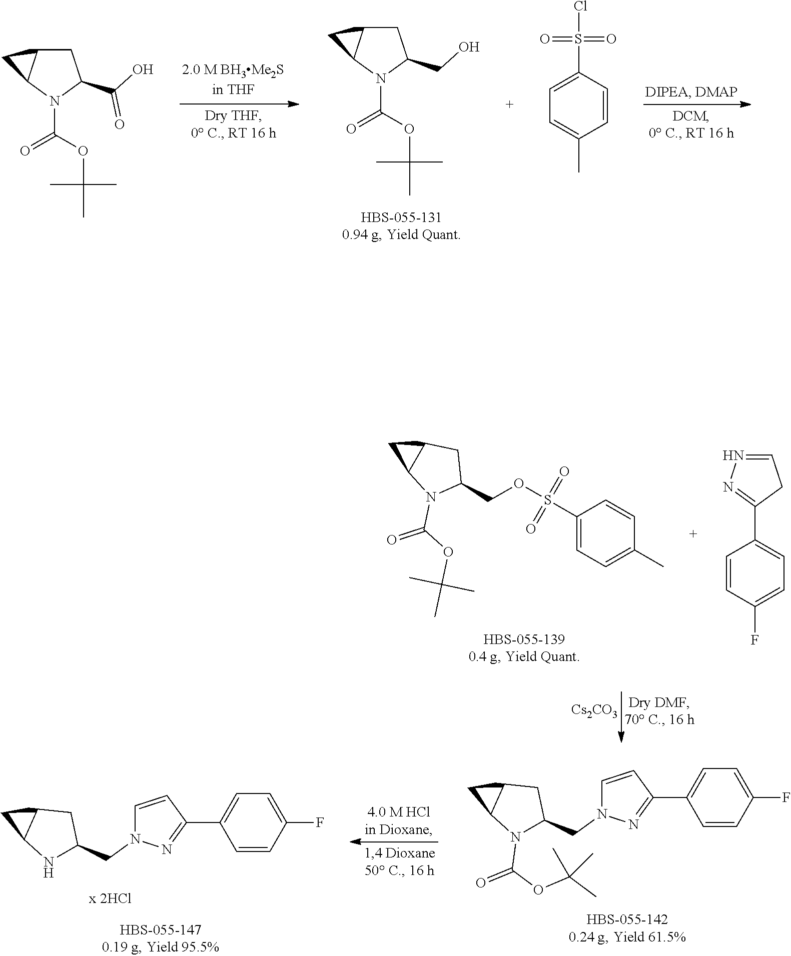

Step 1: Synthesis of HBS-055-131: (1S,3S,5S)-2-(Tert-butoxy carbonyl)-2-azabicyclo [3.1.0] Heptane-3-carboxylic acid (1.0 g, 4.4 mmol) was dissolved in Dry THF (10.0 mL). The 2.0 M BH₃·Me₂S (4.4 mL, 8.8 mmol) was added at 0° C. temperature. The rxn mixture gradually warmed at ambient temperature for 16 h. The LCMS shows product formation m/z 158.1. The rxn mixture was quenched with Methanol. The reaction mixture was diluted with water. The product was extracted with Ethyl acetate. The EtOAc layers were separated and dried over anhydrous Na₂SO₄. The evaporation of solvent gave 0.94 g of crude product. (Yield Quant.). MS (ESI) mass calcd. for C₁₁H₁₉NO₃, 213.27; m/z found 158.1 [M−Boc]⁺.

Step 2: Synthesis of HBS-055-139: Compound HBS-055-131 (0.25 g, 1.17 mmol) was dissolved in DCM (10.0 mL). The DIPEA (0.4 mL, 2.34 mmol) was added followed by DMAP (0.24 g, 2.34 mmol). The rxn mixture was cooled at 0° C. in the Ice bath. The p-TsCl (0.25 g, 1.29 mmol) was added. The rxn mixture was stirred and gradually warmed to room temperature for 16 h. The LCMS shows product formation m/z 268.1, 312.1. The reaction mixture was diluted with water. The product was extracted with DCM. The DCM layer was separated and dried over anhydrous Na₂SO₄. The evaporation of solvent gave 0.4 g of product (Yield Quant.). MS (ESI) mass calcd. for C₁₈H₂₅NO₅S, 367.46; m/z found, 268.1, 312.1 [M−Boc]⁺.

Step 3: Synthesis of HBS-055-142: Compound HBS-055-139 (0.4 g, 1.1 mmol) and 3-(4-Fluoro-phenyl)-1H-pyrazole (0.2 g, 1.21 mmol) were dissolved in Dry DMF (8.0 mL). The anhydrous Cs₂CO₃ (1.1 g, 3.3 mmol) was added and reaction mixture was stirred at 70° C. temperature for 16 h. The LCMS shows product formation m/z 358.2. The rxn mixture was cooled at ambient temperature and diluted with water. The product was extracted with Ethyl acetate. The combined EtOAc layer was separated and dried over anhydrous Na₂SO₄. The evaporation of solvent gave crude product. The crude product was purified by column chromatography, Mobile phase: EtOAc:Hexane gradient. The 0.24 g of pure product was obtained (Yield 61.5%). MS (ESI) mass calcd. for C₂₀H₂₄FN₃O₂, 357.42; m/z found 358.2 [M+H]⁺.

Step 4: Synthesis of HBS-055-147: Compound HBS-055-141 (0.24 g, 0.68 mmol) was dissolved in Dry Dioxane (5.0 mL). The 4.0 M HCl solution in Dioxane (1.35 mL, 2.71 mmol) was added and rxn mixture was stirred at 50° C. temperature for 16 h. LCMS shows product formation m/z 258.1. The rxn mixture was cooled and ppts were filtered. The ppts were dried to obtain 0.19 g of solid product (Yield 95.5%). MS (ESI) mass calcd. for C₁₅H₁₆FN₃, 257.31; m/z found 258.1 [M+H]⁺.

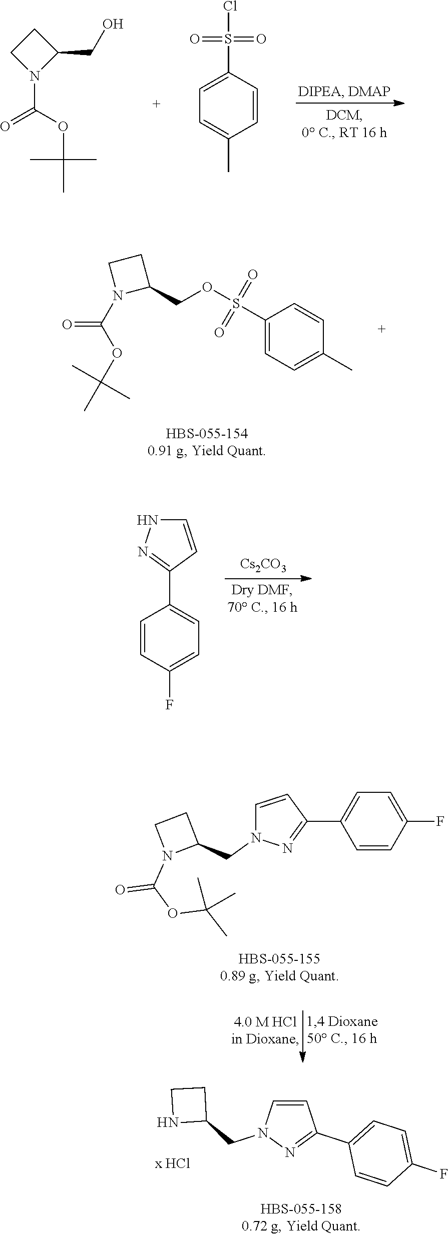

Step 1: Synthesis of HBS-055-154: (S)-1-(tert-Butoxycarbonyl)-2-azetidinemethanol (0.5 g, 2.67 mmol) was dissolved in DCM (10.0 mL). The DIPEA (0.93 mL, 5.34 mmol) was added followed by DMAP (0.65 g, 5.34 mmol). The rxn mixture was cooled at 0° C. in the Ice bath. The p-TsCl (0.56 g, 2.94 mmol) was added. The rxn mixture was stirred and gradually warmed to room temperature for 16 h. The LCMS shows product formation m/z 242.1, 286.1. The reaction mixture was diluted with water. The product was extracted with DCM. The DCM layer was separated and dried over anhydrous Na₂SO₄. The evaporation of solvent gave 0.91 g of product (Yield Quant.). MS (ESI) mass calcd. for C₁₆H₂₃NO₅S, 341.42; m/z found, 242.1, 286.1 [M−Boc]⁺.

Step 2: Synthesis of HBS-055-155: Compound HBS-055-154 (0.91 g, 2.67 mmol) and 3-(4-Fluorophenyl)-1H-pyrazole (0.48 g, 0.48 mmol) were dissolved in Dry DMF (10.0 mL). The anhydrous Cs₂CO₃ (2.61 g, 8.01 mmol) was added, and reaction mixture was stirred at 70° C. temperature for 16 h. The LCMS shows product formation m/z 332.2. The rxn mixture was cooled at ambient temperature and diluted with water. The product was extracted with Ethyl acetate. The combined EtOAc layer was separated and dried over anhydrous Na₂SO₄. The evaporation of solvent gave crude product. The crude product was purified by column chromatography, Mobile phase: EtOAc:Hexane gradient. The 0.89 g of pure product was obtained (Yield Quant.). MS (ESI) mass calcd. for C₁₈H₂₂FN₃O₂, 331.38; m/z found 332.2 [M+H]⁺.

Step 3: Synthesis of HBS-055-158: Compound HBS-055-155 (0.89 g, 2.67 mmol) was dissolved in Dry Dioxane (10.0 mL). The 4.0 M HCl solution in Dioxane (2.7 mL, 10.68 mmol) was added and rxn mixture was stirred at 50° C. temperature for 16 h. LCMS shows product formation m/z 232.1. The rxn mixture was cooled and concentrated under reduced pressure to obtain 0.72 g of solid product (Yield Quant.). MS (ESI) mass calcd. for C₁₃H₁₄FN₃, 231.27; m/z found 232.1 [M+H]⁺.

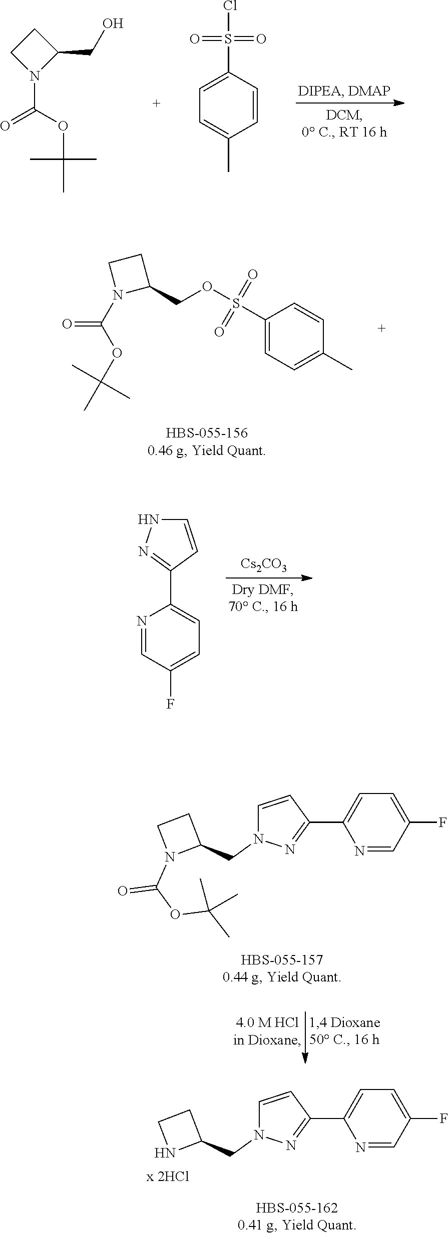

Step 1: Synthesis of HBS-055-156: (S)-1-(tert-Butoxycarbonyl)-2-azetidinemethanol (0.25 g, 1.33 mmol) was dissolved in DCM (8.0 mL). The DIPEA (0.47 mL, 2.67 mmol) was added followed by DMAP (0.33 g, 2.67 mmol). The rxn mixture was cooled at 0° C. in the Ice bath. The p-TsCl (0.28 g, 1.47 mmol) was added. The rxn mixture was stirred and gradually warmed to room temperature for 16 h. The LCMS shows product formation m/z 242.1, 286.1. The reaction mixture was diluted with water. The product was extracted with DCM. The DCM layer was separated and dried over anhydrous Na₂SO₄. The evaporation of solvent gave 0.46 g of product (Yield Quant.). MS (ESI) mass calcd. for C₁₆H₂₃NO₅S, 341.42; m/z found, 242.1, 286.1 [M−Boc]⁺.

Step 2: Synthesis of HBS-055-157: Compound HBS-055-156 (0.46 g, 1.33 mmol) and 5-fluoro-2-(1H-pyrazole-3-yl)pyridine (0.26 g, 1.60 mmol) were dissolved in Dry DMF (10.0 mL). The anhydrous Cs₂CO₃ (1.31 g, 4.0 mmol) was added, and reaction mixture was stirred at 70° C. temperature for 16 h. The LCMS shows product formation m/z 333.1. The rxn mixture was cooled at ambient temperature and diluted with water. The product was extracted with Ethyl acetate. The combined EtOAc layer was separated and dried over anhydrous Na₂SO₄. The evaporation of solvent gave crude product. The crude product was purified by column chromatography, Mobile phase: EtOAc:Hexane gradient. The 0.44 g of pure product was obtained (Yield Quant.). MS (ESI) mass calcd. for C₁₇H₂₁FN₄O₂, 332.37; m/z found 333.1 [M+H]⁺.

Step 3: Synthesis of HBS-055-162: Compound HBS-055-157 (0.44 g, 1.34 mmol) was dissolved in Dry Dioxane (10.0 mL). The 4.0 M HCl solution in Dioxane (1.34 mL, 5.34 mmol) was added and rxn mixture was stirred at 40° C. temperature for 16 h. LCMS shows product formation m/z 233.1. The rxn mixture cooled and concentrated under reduced pressure to obtain 0.41 g of product (Yield Quant.). MS (ESI) mass calcd. for C₁₂H₁₃FN₄, 232.26; m/z found 233.1 [M+H]⁺.

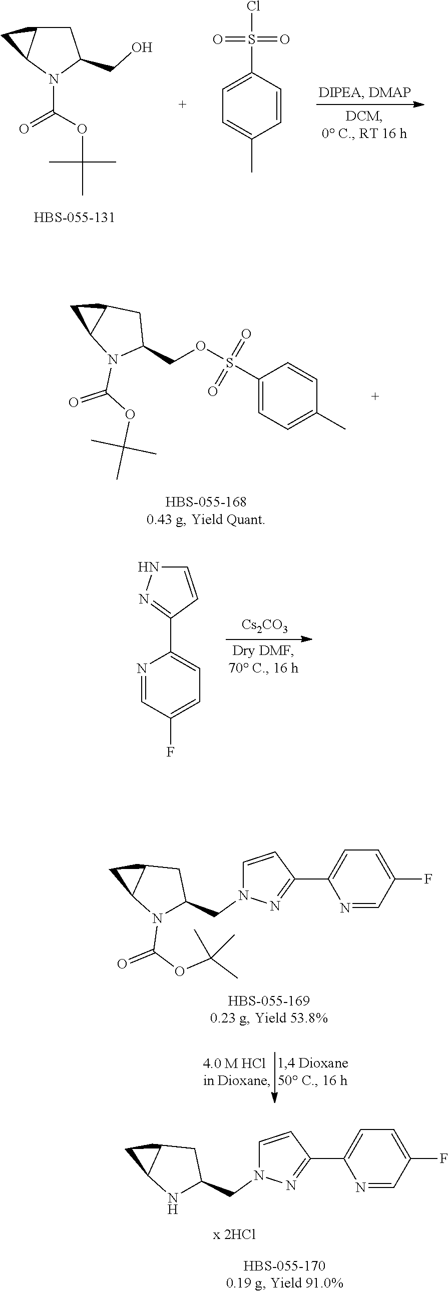

Step 1: Synthesis of HBS-055-168: Compound HBS-055-131 (0.25 g, 1.17 mmol) was dissolved in DCM (10.0 mL). The DIPEA (0.4 mL, 2.34 mmol) was added followed by DMAP (0.24 g, 2.34 mmol). The rxn mixture was cooled at 0° C. in the Ice bath. The p-TsCl (0.25 g, 1.29 mmol) was added. The rxn mixture was stirred and gradually warmed to room temperature for 16 h. The LCMS shows product formation m/z 268.1, 312.1. The reaction mixture was diluted with water. The product was extracted with DCM. The DCM layer was separated and dried over anhydrous Na₂SO₄. The evaporation of solvent gave 0.43 g of product (Yield Quant.). MS (ESI) mass calcd. for C₁₈H₂₅NO₅S, 367.46; m/z found, 268.1, 312.1 [M−Boc]⁺.

Step 2: Synthesis of HBS-055-169: Compound HBS-055-168 (0.43 g, 1.17 mmol) and 5-fluoro-2-(1H-pyrazol-3-yl)pyridine (0.23 g, 1.41 mmol) were dissolved in Dry DMF (8.0 mL). The anhydrous Cs₂CO₃ (1.15 g, 3.52 mmol) was added, and reaction mixture was stirred at 70° C. temperature for 16 h. The LCMS shows product formation m/z 359.2. The rxn mixture was cooled at ambient temperature and diluted with water. The product was extracted with Ethyl acetate. The combined EtOAc layer was separated and dried over anhydrous Na₂SO₄. The evaporation of solvent gave crude product. The crude product was purified by column chromatography, Mobile phase: EtOAc:Hexane gradient. The 0.23 g of pure product was obtained (Yield 53.8%). MS (ESI) mass calcd. for C₁H₂₃FN₄O₂, 358.41; m/z found 359.2 [M+H]⁺.

Step 3: Synthesis of HBS-055-170: Compound HBS-055-169 (0.23 g, 0.63 mmol) was dissolved in Dry Dioxane (6.0 mL). The 4.0 M HCl solution in Dioxane (0.63 mL, 2.52 mmol) was added and rxn mixture was stirred at 50° C. temperature for 16 h. LCMS shows product formation m/z 259.1. The rxn mixture was cooled and ppts were filtered. The ppts were dried to obtain 0.19 g of solid product (Yield 90.95%). MS (ESI) mass calcd. for C₁₄H₁₅FN₄, 258.29; m/z found 259.1 [M+H]⁺.

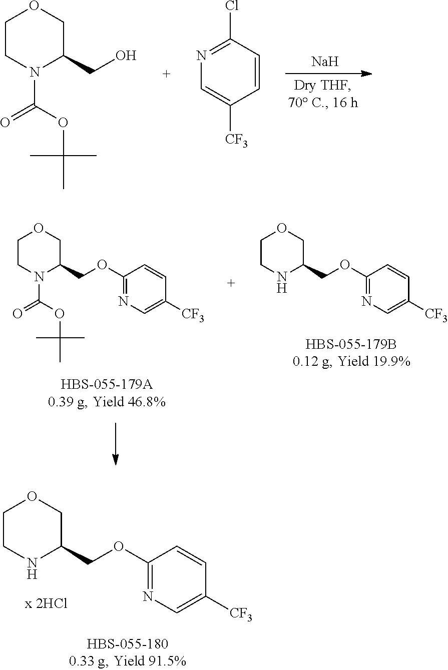

Step 1: Synthesis of HBS-055-179: The (R)-3-hydroxymethyl-4-Boc-morpholine (0.5 g, 2.3 mmol) was dissolved in Dry THF (10.0 mL). The NaH (0.14 g, 3.45 mmol) was added at 0° C. temperature. The 2-Chloro-5-trifluoromethylpyridine (0.5 g, 2.76 mmol) was added and the reaction mixture was gradually heated at reflux temperature for 16 h. The LCMS shows two product formation m/z 363.1 and m/z 263.1. The rxn mixture was diluted with water. The product was extracted with Ethyl acetate. The combined EtOAc layers were separated and dried over anhydrous Na₂SO₄. The evaporation of solvent gave crude product. The crude product was purified by column chromatography, Mobile phase: EtOAc:Hexane gradient and DCM:MeOH gradient. The 0.39 g of pure product A was obtained (Yield 46.8%) and 0.12 g of deboc product B was obtained (Yield 19.9%). MS (ESI) mass calcd. for C₁₆H₂₁F₃N₂O₄, 362.34; m/z found 363.1 [M+H]⁺ and MS (ESI) mass calcd. for C₁₁H₁₃F₃N₂O₂, 262.23; m/z found 263.1 [M+H]⁺

Step 2: Synthesis of HBS-055-180: Compound HBS-055-179A (0.39 g, 1.08 mmol) was dissolved in Dioxane (6.0 mL). The 4.0 M HCl solution in dioxane (1.1 mL, 4.31 mmol) was added and rxn mixture was stirred at 50° C. temperature for 16 h. LCMS shows product formation m/z 263.0. The rxn mixture was cooled and ppts were filtered. The ppts were dried to obtain 0.33 g of pure product (Yield 91.5%). MS (ESI) mass calcd. for C₁₁H₁₃F₃N₂O₂, 262.23; m/z found 263.0 [M+H]⁺.

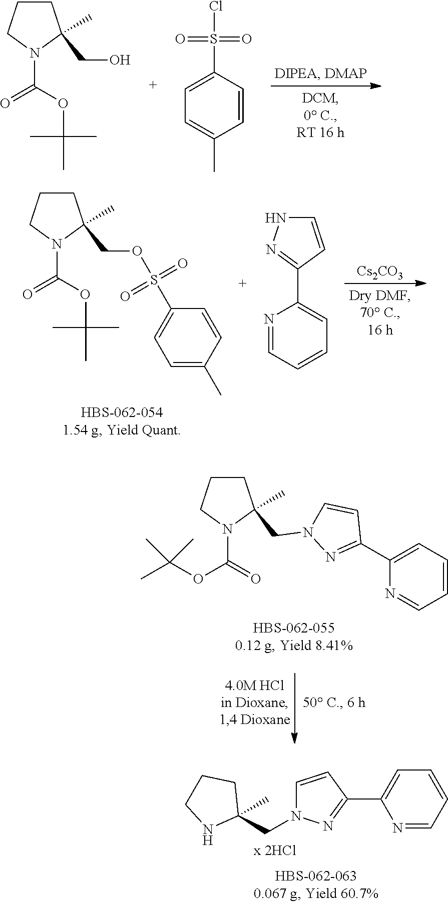

Step 1: Synthesis of HBS-062-054: Compound HBS-062-051 (0.9 g, 4.18 mmol) was dissolved in DCM (15.0 mL). The DIPEA (1.1 mL, 6.27 mmol) was added followed by DMAP (0.77 g, 6.27 mmol). The rxn mixture was cooled at 0° C. in the Ice bath. The p-TsCl (0.88 g, 4.6 mmol) was added. The rxn mixture was stirred and gradually warmed to room temperature for 16 h. The LCMS shows product formation m/z 270.1, 314.1. The reaction mixture was diluted with water. The product was extracted with DCM. The DCM layer was separated and dried over anhydrous Na₂SO₄. The evaporation of solvent gave 1.54 g of product (Yield Quant.). MS (ESI) mass calcd. for C₁₈H₂₇NO₅S, 369.48; m/z found, 270.1, 314.1 [M−Boc]⁺.

Step 2: Synthesis of HBS-062-055: Compound HBS-062-054 (1.54 g, 4.17 mmol) and 2-(1H-pyrazol-3-yl)pyridine (0.61 g, 4.17 mmol) were dissolved in Dry DMF (12.0 mL). The anhydrous Cs₂CO₃ (4.1 g, 12.5 mmol) was added, and reaction mixture was stirred at 70° C. temperature for 12 h. The LCMS shows product formation m/z 343.2 and other side products. The rxn mixture was cooled at ambient temperature and diluted with water. The product was extracted with Ethyl acetate. The combined EtOAc layer was separated and dried over anhydrous Na₂SO₄. The evaporation of solvent gave crude product. The crude product was purified by column chromatography, Mobile phase: EtOAc:Hexane gradient. The 0.12 g of pure product was obtained (Yield 8.41%). MS (ESI) mass calcd. for C₁₉H₂₆N₄O₂, 342.44; m/z found 343.2 [M+H]⁺.

Step 3: Synthesis of HBS-062-063: Compound HBS-062-055 (0.12 g, 0.35 mmol) was dissolved in Dry Dioxane (5.0 mL). The 4.0 M HCl solution in Dioxane (0.4 mL, 1.75 mmol) was added and rxn mixture was stirred at 50° C. temperature for 6 h. LCMS shows product formation m/z 243.1. The rxn mixture was cooled and solvent was concentrated to obtain 0.067 g of solid product (Yield 60.7%). MS (ESI) mass calcd. for C₁₄H₁₈N₄, 242.32; m/z found 243.1 [M+H]⁺.

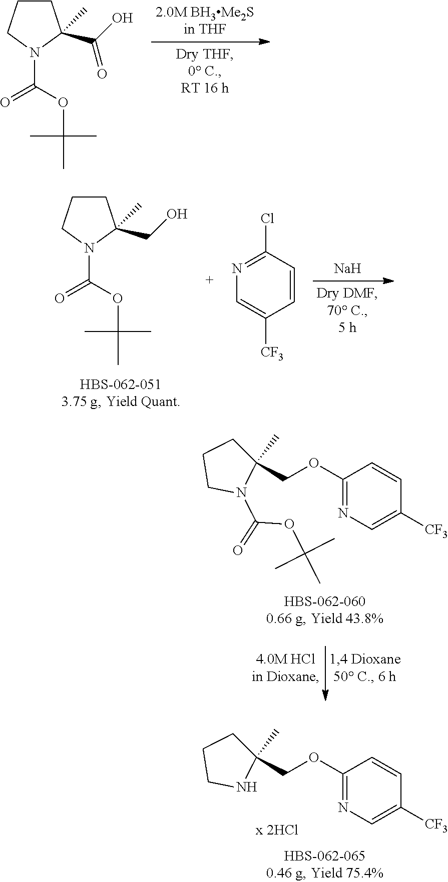

Step 1: Synthesis of HBS-062-051: N-Boc-alpha-methyl-L-proline (4.0 g, 17.44 mmol) was dissolved in Dry THF (20.0 mL). The 2.0 M BH₃·Me₂S (17.4 mL, 34.9 mmol) was added at 0° C. temperature. The rxn mixture gradually warmed at ambient temperature for 16 h. The LCMS shows product formation m/z 160.1. The rxn mixture was quenched with Methanol. The reaction mixture was diluted with water. The product was extracted with Ethyl acetate. The EtOAc layers were separated and dried over anhydrous Na₂SO₄. The evaporation of solvent gave 3.75 g of crude product. (Yield Quant.). MS (ESI) mass calcd. for C₁₁H₂₁NO₃, 215.29; m/z found 160.1 [M−Boc]⁺.

Step 2: Synthesis of HBS-062-060: Compound HBS-062-051 (0.9 g, 4.18 mmol) was dissolved in Dry DMF (10.0 mL). The NaH (0.25 g, 6.27 mmol) was added at 0° C. temperature. The 2-Chloro-5-trifluoromethylpyridine (0.76 g, 4.18 mmol) was added and the reaction mixture was gradually heated at reflux temperature for 5 h. The LCMS shows product formation m/z 361.1. The rxn mixture was diluted with water. The product was extracted with Ethyl acetate. The combined EtOAc layers were separated and dried over anhydrous Na₂SO₄. The evaporation of solvent gave crude product. The crude product was purified by column chromatography, Mobile phase: EtOAc:Hexane gradient. The 0.66 g of product was obtained (Yield 43.8%). MS (ESI) mass calcd. for C₁₇H₂₃F₃N₂O₃, 360.37; m/z found 361.1 [M+H]⁺.

Step 3: Synthesis of HBS-062-065: Compound HBS-062-060 (0.66 g, 1.83 mmol) was dissolved in Dioxane (8.0 mL). The 4.0 M HCl solution in dioxane (1.83 mL, 7.33 mmol) was added and rxn mixture was stirred at 50° C. temperature for 16 h. LCMS shows product formation m/z 261.1. The rxn mixture was cooled and ppts were filtered. The ppts were dried to obtain 0.46 g of pure product (Yield 75.4%). MS (ESI) mass calcd. for C₁₂H₁₅F₃N₂O, 260.26; m/z found 261.1 [M+H]⁺.

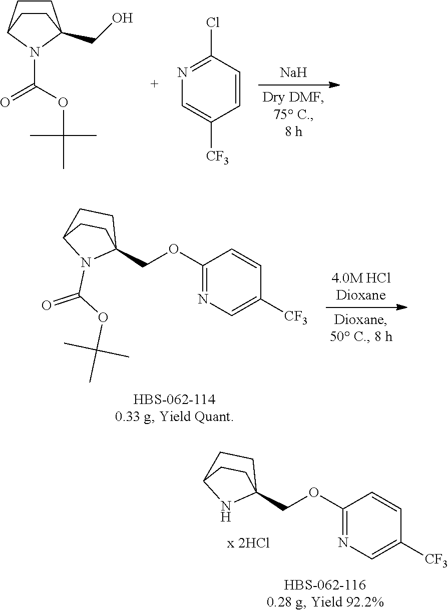

Step 1: Synthesis of HBS-062-114: Tert-butyl-1-(hydroxymethyl)-7-azabicyclo[2.2.1]heptane-7-carboxylate (0.2 g, 0.88 mmol) was dissolved in Dry DMF (6.0 mL). The NaH (0.052 g, 1.32 mmol) was added at 0° C. temperature. The 2-Chloro-5-trifluoromethylpyridine (0.24 g, 1.32 mmol) was added and the reaction mixture was gradually heated at 75° C. temperature for 8 h. The LCMS shows product formation m/z 373.1. The rxn mixture was diluted with water. The product was extracted with Ethyl acetate. The combined EtOAc layers were separated and dried over anhydrous Na₂SO₄. The evaporation of solvent gave crude product. The crude product was purified by column chromatography, Mobile phase: EtOAc:Hexane gradient. The 0.33 g of product was obtained (Yield Quant.). MS (ESI) mass calcd. for C₁₈H₂₃F₃N₂O₃, 372.38; m/z found 373.1 [M+H]⁺.

Step 2: Synthesis of HBS-062-116: Compound HBS-062-114 (0.33 g, 0.88 mmol) was dissolved in Dioxane (5.0 mL). The 4.0 M HCl solution in dioxane (0.88 mL, 3.52 mmol) was added and rxn mixture was stirred at 50° C. temperature for 8 h. LCMS shows product formation m/z 273.1. The rxn mixture was cooled and concentrated under reduced pressure to obtain 0.28 g of pure product (Yield 92.2%). MS (ESI) mass calcd. for C₁₃H₁₅F₃N₂O, 272.27; m/z found 273.1 [M+H]⁺.

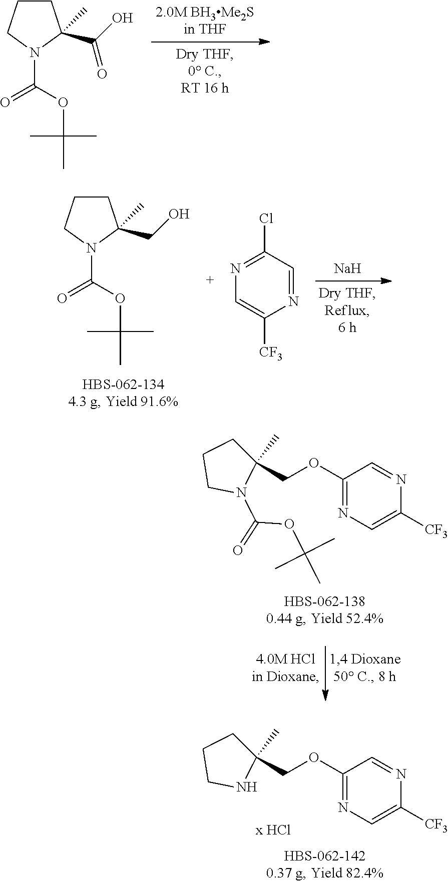

Step 1: Synthesis of HBS-062-134: N-Boc-alpha-methyl-L-proline (5.0 g, 21.81 mmol) was dissolved in Dry THF (20.0 mL). The 2.0 M BH₃·Me₂S (21.8 mL, 43.62 mmol) was added at 0° C. temperature. The rxn mixture gradually warmed at ambient temperature for 16 h. The LCMS shows product formation m/z 160.0. The rxn mixture was quenched with Methanol. The reaction mixture was diluted with water. The product was extracted with Ethyl acetate. The EtOAc layers were separated and dried over anhydrous Na₂SO₄. The evaporation of solvent gave 4.3 g of crude product. (Yield Quant.). MS (ESI) mass calcd. for C₁₁H₂₁NO₃, 215.29; m/z found 160.1 [M−Boc]⁺.

Step 2: Synthesis of HBS-062-138: Compound HBS-062-134 (0.5 g, 2.32 mmol) was dissolved in Dry THF (6.0 mL). The NaH (0.14 g, 3.49 mmol) was added at 0° C. temperature. The 2-Chloro-5-trifluoromethylpyrazine (0.3 mL, 2.32 mmol) was added and the reaction mixture was gradually heated at reflux temperature for 6 h. The LCMS shows product formation m/z 262.1, 306.0. The rxn mixture was diluted with water. The product was extracted with Ethyl acetate. The combined EtOAc layers were separated and dried over anhydrous Na₂SO₄. The evaporation of solvent gave crude product. The crude product was purified by column chromatography, Mobile phase: EtOAc:Hexane gradient. The 0.44 g of product was obtained (Yield 52.4%). MS (ESI) mass calcd. for C₁₆H₂₂F₃N₃O₃, 361.36; m/z found 262.1, 306.0 [M−Boc]⁺.

Step 3: Synthesis of HBS-062-142: Compound HBS-062-138 (0.44 g, 1.22 mmol) was dissolved in Dioxane (5.0 mL). The 4.0 M HCl solution in dioxane (1.52 mL, 6.1 mmol) was added and rxn mixture was stirred at 50° C. temperature for 8 h. LCMS shows product formation m/z 262.1. The rxn mixture was cooled and concentrated under reduced pressure to obtain 0.37 g of pure product (Yield 82.4%). MS (ESI) mass calcd. for C₁₁H₁₄F₃N₃O, 261.24; m/z found 262.1 [M+H]⁺.

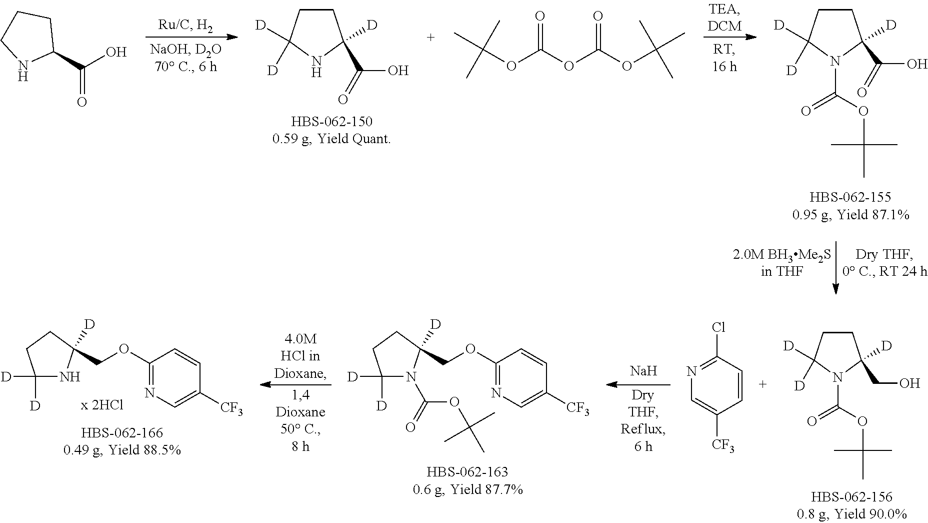

Step 1: Synthesis of HBS-062-150: The L-proline (0.58 g, 5.0 mmol) and NaOH (0.6 g, 15.0 mmol) were dissolved in D₂O (5.0 mL). The Ru/C, 5 wt. % (0.058 g, 10% w/w) was added and reaction mixture was stirred under hydrogen atm at 70° C. temperature for 6 h. LCMS shows product formation m/z 118.1. The rxn mixture was filtered over celite bed and washed with D₂O. The reaction mixture pH was adjusted to pH 6.5 using HCl. The Dowex X-8 [H₊] resin was added and aq. layer filtered and washed with 25% aq. ammonia solution. The aq. layer was concentrated under reduced pressure to obtain 0.59 g of crude product. (Yield Quant.). MS (ESI) mass calcd. for C₅H₆D₃NO₂, 118.15; m/z found 118.1 [M+H]⁺.

Step 2: Synthesis of HBS-062-155: Compound HBS-062-150 (0.59 g, 5.0 mmol) was dissolved in DCM (15.0 mL). The triethyl amine (0.76 mL, 5.5 mmol) was added followed by Di-tert-butyl-dicarbonate (1.2 g, 5.5 mmol). The rxn mixture was stirred at ambient temperature for 16 h. The LCMS shows product formation m/z 162.0. The reaction mixture was diluted with water. The product was extracted with DCM. The DCM layers were separated and dried over anhydrous Na₂SO₄. The evaporation of solvent and aq. layer extracted gave 0.95 g combined product. (Yield 87.1%). MS (ESI) mass calcd. for C₁₀H₁₄D₃NO₄, 218.26; m/z found 162.0 [M−tButyl]⁺.

Step 3: Synthesis of HBS-062-156: Compound HBS-062-155 (0.95 g, 4.35 mmol) was dissolved in Dry THF (10.0 mL). The 2.0 M BH₃·Me₂S (4.3 mL, 8.70 mmol) was added at 0° C. temperature. The rxn mixture gradually warmed at ambient temperature for 16 h. The LCMS shows product formation m/z 148.1. The rxn mixture was quenched with Methanol. The reaction mixture was diluted with water. The product was extracted with Ethyl acetate. The EtOAc layers were separated and dried over anhydrous Na₂SO₄. The crude product was purified by column chromatography, Mobile phase: EtOAc:Hexane gradient. The 0.8 g of product was obtained. (Yield 90.0%). MS (ESI) mass calcd. for C₁₀H₁₆D₃NO₃, 204.28; m/z found 148.1 [M−tButyl]⁺.

Step 4: Synthesis of HBS-062-163: Compound HBS-062-156 (0.4 g, 1.96 mmol) was dissolved in Dry THF (10.0 mL). The NaH (0.12 g, 2.49 mmol) was added at 0° C. temperature. The 2-Chloro-5-trifluoromethylpyridine (0.53 g, 2.94 mmol) was added and the reaction mixture was gradually heated at reflux temperature for 6 h. The LCMS shows product formation m/z 350.1. The rxn mixture was diluted with water. The product was extracted with Ethyl acetate. The combined EtOAc layers were separated and dried over anhydrous Na₂SO₄. The evaporation of solvent gave crude product. The crude product was purified by column chromatography, Mobile phase: EtOAc:Hexane gradient. The 0.6 g of product was obtained (Yield 87.7%). MS (ESI) mass calcd. for C₁₆H₁₈D₃F₃N₂O₃, 349.36; m/z found 350.1 [M+H]⁺.

Step 5: Synthesis of HBS-062-166: Compound HBS-062-163 (0.6 g, 1.72 mmol) was dissolved in Dioxane (8.0 mL). The 4.0 M HCl solution in dioxane (2.15 mL, 8.59 mmol) was added and rxn mixture was stirred at 55° C. temperature for 8 h. LCMS shows product formation m/z 250.1. The rxn mixture was cooled at ambient temperature. The ppts were filtered and dried to obtain 0.49 g of pure product (Yield 88.5%). MS (ESI) mass calcd. for C₁₁H₁₀D₃F₃N₂O, 249.25; m/z found 250.1 [M+H]⁺.

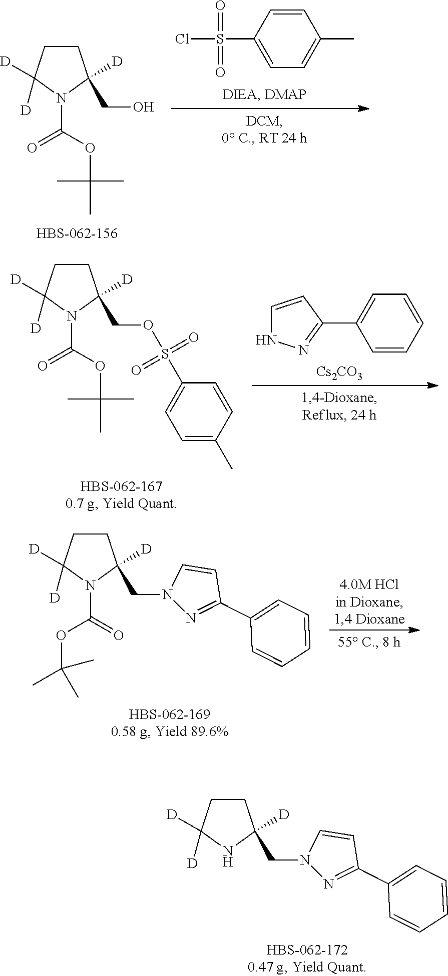

Step 1: Synthesis of HBS-062-167: Compound HBS-062-156 (0.4 g, 1.96 mmol) was dissolved in DCM (15.0 mL). The DIPEA (0.51 mL, 2.94 mmol) was added followed by DMAP (0.36 g, 2.94 mmol). The rxn mixture was cooled at 0° C. in the Ice bath. The p-TsCl (0.41 g, 2.15 mmol) was added. The rxn mixture was stirred and gradually warmed to room temperature for 24 h. The LCMS shows product formation m/z 258.1, 302.1. The reaction mixture was diluted with water. The product was extracted with DCM. The DCM layer was separated and dried over anhydrous Na₂SO₄. The evaporation of solvent gave 0.7 g of product (Yield Quant.). MS (ESI) mass calcd. for C₁₇H₂₂D₃NO₅S, 358.47; m/z found, 258.1, 302.1 [M−Boc]⁺.

Step 2: Synthesis of HBS-062-169: Compound HBS-062-167 (0.7 g, 1.96 mmol) and 3-phenyl-1H-pyrazol (0.34 g, 2.35 mmol) were dissolved in Dry 1,4-Dioxane (10.0 mL). The anhydrous Cs₂CO₃ (1.6 g, 4.9 mmol) was added, and reaction mixture was stirred at reflux temperature for 24 h. The LCMS shows product formation m/z 331.2. The rxn mixture was cooled at ambient temperature and diluted with water. The product was extracted with Ethyl acetate. The combined EtOAc layer was separated and dried over anhydrous Na₂SO₄. The evaporation of solvent gave crude product. The crude product was purified by column chromatography, Mobile phase: EtOAc:Hexane gradient. The 0.58 g of pure product was obtained (Yield 89.6%). MS (ESI) mass calcd. for C₁₉H₂₂D₃N₃O₂, 330.44; m/z found 331.2 [M+H]⁺.

Step 3: Synthesis of HBS-062-172: Compound HBS-062-169 (0.58 g, 1.76 mmol) was dissolved in Dry Dioxane (8.0 mL). The 4.0 M HCl solution in Dioxane (2.2 mL, 8.78 mmol) was added and rxn mixture was stirred at 55° C. temperature for 8 h. LCMS shows product formation m/z 231.1. The rxn mixture was cooled and solvent was concentrated to obtain 0.47 g of solid product (Yield Quant.). MS (ESI) mass calcd. for C₁₄H₁₄D₃N₃, 230.32; m/z found 231.1 [M+H]⁺.

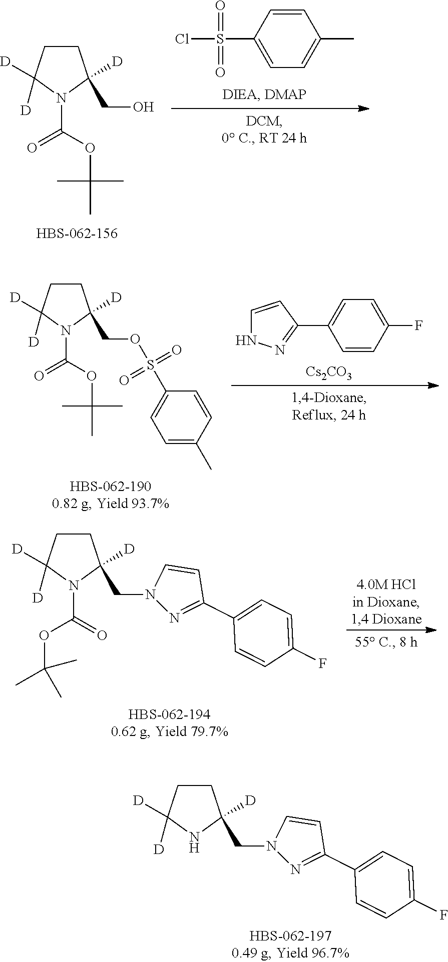

Step 1: Synthesis of HBS-062-190: Compound HBS-062-156 (0.5 g, 2.45 mmol) was dissolved in DCM (15.0 mL). The DIPEA (0.64 mL, 3.67 mmol) was added followed by DMAP (0.45 g, 3.67 mmol). The rxn mixture was cooled at 0° C. in the Ice bath. The p-TsCl (0.56 g, 2.94 mmol) was added. The rxn mixture was stirred and gradually warmed to room temperature for 24 h. The LCMS shows product formation m/z 259.1, 303.1. The reaction mixture was diluted with water. The product was extracted with DCM. The DCM layer was separated and dried over anhydrous Na₂SO₄. The evaporation of solvent gave 0.82 g of product (Yield 93.7%). MS (ESI) mass calcd. for C₁₇H₂₂D₃NO₅S, 358.47; m/z found, 259.1, 303.1 [M−Boc]⁺.

Step 2: Synthesis of HBS-062-194: Compound HBS-062-190 (0.8 g, 2.23 mmol) and 3-(4-fluorophenyl)-1H-pyrazol (0.43 g, 2.68 mmol) were dissolved in Dry 1,4-Dioxane (12.0 mL). The anhydrous Cs₂CO₃ (1.81 g, 5.58 mmol) was added, and reaction mixture was stirred at reflux temperature for 24 h. The LCMS shows product formation m/z 349.2. The rxn mixture was cooled at ambient temperature and diluted with water. The product was extracted with Ethyl acetate. The combined EtOAc layer was separated and dried over anhydrous Na₂SO₄. The evaporation of solvent gave crude product. The crude product was purified by column chromatography, Mobile phase: EtOAc:Hexane gradient. The 0.62 g of pure product was obtained (Yield 79.7%). MS (ESI) mass calcd. for C₁₉H₂₁D₃FN₃O₂, 348.43; m/z found 349.2 [M+H]⁺.

Step 3: Synthesis of HBS-062-197: Compound HBS-062-194 (0.62 g, 1.78 mmol) was dissolved in Dry Dioxane (8.0 mL). The 4.0 M HCl solution in Dioxane (1.8 mL, 7.12 mmol) was added and rxn mixture was stirred at 55° C. temperature for 8 h. LCMS shows product formation m/z 249.2. The rxn mixture was cooled and ppts were filtered. The drying of ppts gave 0.49 g of solid product (Yield 96.7%). MS (ESI) mass calcd. for C₁₄H₁₃D₃FN₃, 248.31; m/z found 249.2 [M+H]⁺.

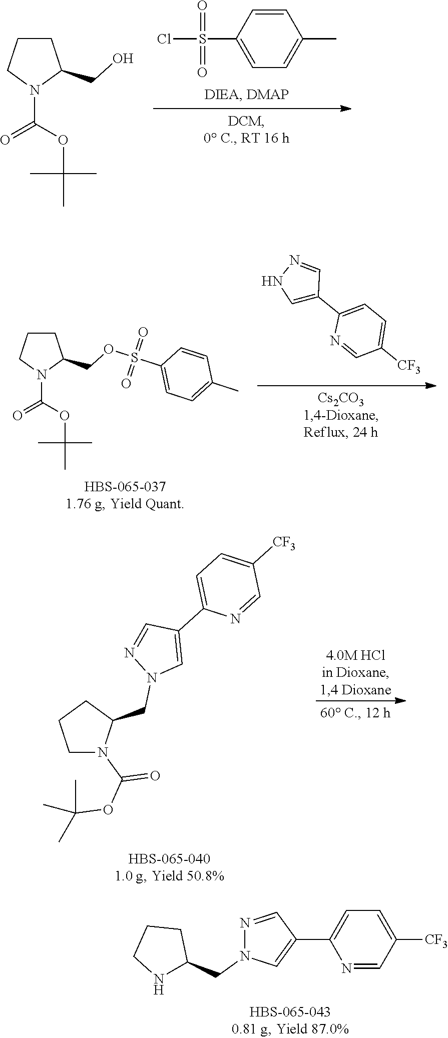

Step 1: Synthesis of HBS-065-037: The N-Boc-L-prolinol (1.0 g, 4.97 mmol) was dissolved in DCM (15.0 mL). The DIPEA (1.3 mL, 7.45 mmol) was added followed by DMAP (0.91 g, 7.45 mmol). The rxn mixture was cooled at 0° C. in the Ice bath. The p-TsCl (1.04 g, 5.46 mmol) was added. The rxn mixture was stirred and gradually warmed to room temperature for 16 h. The LCMS shows product formation m/z 256.0, 300.0. The reaction mixture was diluted with water. The product was extracted with DCM. The DCM layer was separated and dried over anhydrous Na₂SO₄. The evaporation of solvent gave 1.76 g of product (Yield Quant.). MS (ESI) mass calcd. for C₁₇H₂₅NO₅S, 355.45; m/z found, 256.0, 300.0 [M−Boc]⁺.

Step 2: Synthesis of HBS-065-040: Compound HBS-065-037 (1.76 g, 4.97 mmol) and 2-(1H-pyrazole-4-yl-5-(trifluoromethyl)pyridine hydrochloride (1.42 g, 4.97 mmol) were dissolved in Dry 1,4-Dioxane (25.0 mL). The anhydrous Cs₂CO₃ (3.24 g, 9.94 mmol) was added, and reaction mixture was stirred at reflux temperature for 24 h. The LCMS shows product formation m/z 397.2. The rxn mixture was cooled at ambient temperature and diluted with water. The product was extracted with Ethyl acetate. The combined EtOAc layer was separated and dried over anhydrous Na₂SO₄. The evaporation of solvent gave crude product. The crude product was purified by column chromatography, Mobile phase: EtOAc:Hexane gradient. The 1.0 g of pure product was obtained (Yield 50.8%). MS (ESI) mass calcd. for C₁₉H₂₃F₃N₄O₂, 396.41; m/z found 397.2 [M+H]⁺.

Step 3: Synthesis of HBS-065-043: Compound HBS-065-040 (1.0 g, 2.52 mmol) was dissolved in Dry Dioxane (15.0 mL). The 4.0 M HCl solution in Dioxane (2.52 mL, 10.1 mmol) was added and reaction mixture was stirred at 60° C. temperature for 12 h. LCMS shows product formation m/z 297.1. The rxn mixture was cooled and ppts were filtered. The drying of ppts gave 0.81 g of solid product (Yield 87.0%). MS (ESI) mass calcd. for C₁₄H₁₅F₃N₄, 296.29; m/z found 297.1 [M+H]⁺.

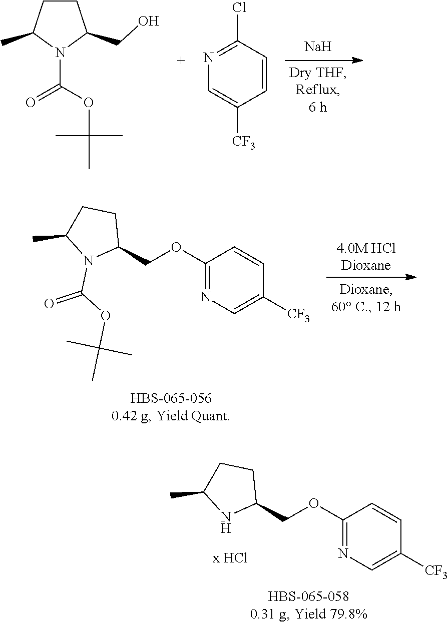

Step 1: Synthesis of HBS-065-056: The (2S,5S)-tert-Butyl-2-(Hydroxymethyl)-5-Methyl pyrrolidine-1-carboxylate (0.25 g, 1.16 mmol) was dissolved in Dry THF (6.0 mL). The NaH (0.07 g, 1.74 mmol) was added at 0° C. temperature. The 2-Chloro-5-trifluoromethylpyridine (0.25 g, 1.39 mmol) was added and the reaction mixture was gradually heated at reflux temperature for 6 h. The LCMS shows product formation m/z 361.2. The rxn mixture was diluted with water. The product was extracted with Ethyl acetate. The combined EtOAc layers were separated and dried over anhydrous Na₂SO₄. The evaporation of solvent gave crude product. The crude product was purified by column chromatography, Mobile phase: EtOAc:Hexane gradient. The 0.42 g of product was obtained (Yield Quant.). MS (ESI) mass calcd. for C₁₇H₂₃F₃N₂O₃, 360.37; m/z found 361.2 [M+H]⁺.

Step 2: Synthesis of HBS-065-058: Compound HBS-065-056 (0.42 g, 1.17 mmol) was dissolved in Dioxane (8.0 mL). The 4.0 M HCl solution in dioxane (1.16 mL, 4.66 mmol) was added and rxn mixture was stirred at 60° C. temperature for 12 h. LCMS shows product formation m/z 261.1. The reaction mixture was cooled at ambient temperature. The ppts were filtered and dried to obtain 0.31 g of pure product (Yield 79.8%). MS (ESI) mass calcd. for C₁₂H₁₅F₃N₂O, 260.26; m/z found 261.1 [M+H]⁺.

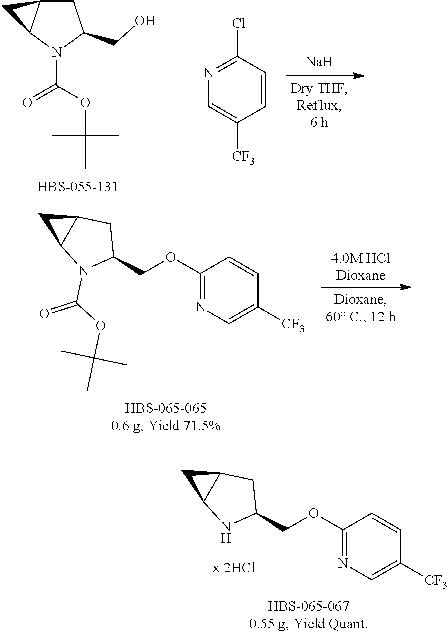

Step 1: Synthesis of HBS-065-065: Compound HBS-055-131 (0.5 g, 2.34 mmol) was dissolved in Dry THF (8.0 mL). The NaH (0.14 g, 3.51 mmol) was added at 0° C. temperature. The 2-Chloro-5-trifluoromethylpyridine (0.51 g, 2.81 mmol) was added and the reaction mixture was gradually heated at reflux temperature for 6 h. The LCMS shows product formation m/z 359.1. The rxn mixture was diluted with water. The product was extracted with Ethyl acetate. The combined EtOAc layers were separated and dried over anhydrous Na₂SO₄. The evaporation of solvent gave crude product. The crude product was purified by column chromatography, Mobile phase: EtOAc:Hexane gradient. The 0.6 g of product was obtained (Yield 71.5%). MS (ESI) mass calcd. for C₁₇H₂₁F₃N₂O₃, 358.36; m/z found 359.1 [M+H]⁺.

Step 2: Synthesis of HBS-065-067: Compound HBS-065-065 (0.6 g, 1.67 mmol) was dissolved in Dioxane (6.0 mL). The 4.0 M HCl solution in dioxane (1.67 mL, 6.7 mmol) was added and rxn mixture was stirred at 60° C. temperature for 12 h. LCMS shows product formation m/z 259.0. The reaction mixture was cooled at ambient temperature and concentrated under reduced pressure to obtain 0.55 g of pure product (Yield Quant.). MS (ESI) mass calcd. for C₁₂H₁₃F₃N₂O, 258.24; m/z found 259.0 [M+H]⁺.

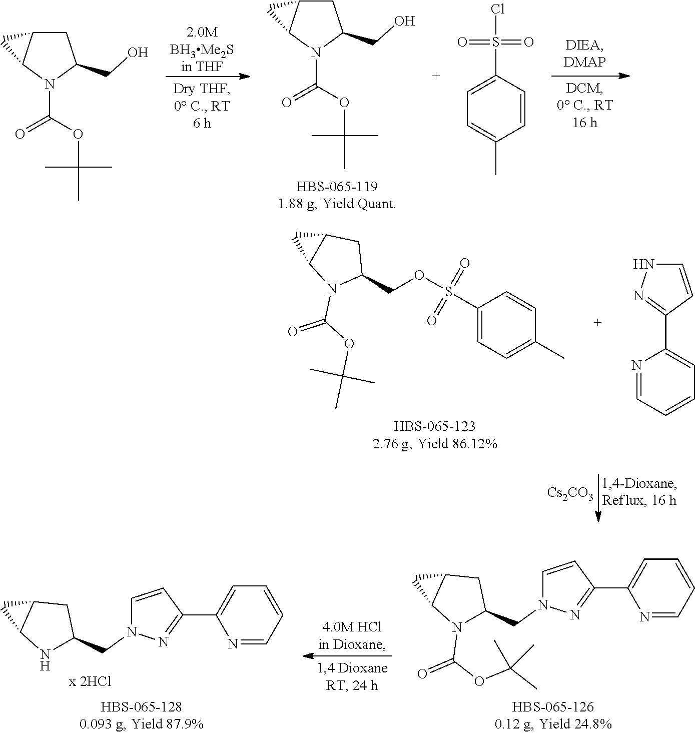

Step 1: Synthesis of HBS-065-119: (1R,3S,5R)-2-[(Tert-butoxy) carbonyl]-2-azabicyclo [3.1.0] Hexane-3-carboxylic acid (2.0 g, 8.8 mmol) was dissolved in Dry THF (20.0 mL). The 2.0 M BH₃·Me₂S (8.8 mL, 17.6 mmol) was added at 0° C. temperature. The rxn mixture gradually warmed at ambient temperature for 16 h. The LCMS shows product formation m/z 158.1. The rxn mixture was quenched with Methanol. The reaction mixture was diluted with water. The product was extracted with Ethyl acetate. The EtOAc layers were separated and dried over anhydrous Na₂SO₄. The evaporation of solvent gave 1.88 g of crude product. (Yield Quant.). MS (ESI) mass calcd. for C₁₁H₁₉NO₃, 213.27; m/z found 158.1 [M−Boc]⁺.

Step 2: Synthesis of HBS-065-123: Compound HBS-065-119 (1.86 g, 8.72 mmol) was dissolved in DCM (25.0 mL). The DIPEA (2.3 mL, 13.1 mmol) was added followed by DMAP (1.6 g, 13.1 mmol). The rxn mixture was cooled at 0° C. in the Ice bath. The p-TsCl (2.0 g, 10.47 mmol) was added. The rxn mixture was stirred and gradually warmed to room temperature for 16 h. The LCMS shows product formation m/z 268.1, 312.1. The reaction mixture was diluted with water. The product was extracted with DCM. The DCM layer was separated and dried over anhydrous Na₂SO₄. The evaporation of solvent gave crude product. The crude product was purified by column chromatography, Mobile phase: EtOAc:Hexane gradient. The 2.76 g of product was obtained (Yield 86.12%). MS (ESI) mass calcd. for C₁₈H₂₅NO₅S, 367.46; m/z found, 268.1, 312.1 [M−Boc]⁺.

Step 3: Synthesis of HBS-065-126: Compound HBS-065-123 (0.5 g, 1.36 mmol) and 2-(1H-Pyrazol-3-yl)pyridine (0.22 g, 1.5 mmol) were dissolved in 1,4-Dioxane (8.0 mL). The anhydrous Cs₂CO₃ (0.89 g, 2.72 mmol) was added, and reaction mixture was stirred at reflux for 16 h. The LCMS shows product formation m/z 341.2. The rxn mixture was cooled at ambient temperature and diluted with water. The product was extracted with Ethyl acetate. The combined EtOAc layer was separated and dried over anhydrous Na₂SO₄. The evaporation of solvent gave crude product. The crude product was purified by column chromatography, Mobile phase: EtOAc:Hexane gradient. The 0.12 g of pure product was obtained (Yield 24.8%). MS (ESI) mass calcd. for C₁₉H₂₄N₄O₂, 340.42; m/z found 341.2 [M+H]⁺.

Step 4: Synthesis of HBS-065-128: Compound HBS-065-126 (0.12 g, 0.33 mmol) was dissolved in Dry Dioxane (5.0 mL). The 4.0 M HCl solution in Dioxane (0.33 mL, 1.35 mmol) was added and rxn mixture was stirred at ambient temperature for 24 h. LCMS shows product formation m/z 241.1. The rxn mixture was cooled and ppts were filtered. The ppts were dried to obtain 0.093 g of solid product (Yield 87.9%). MS (ESI) mass calcd. for C₁₄H₁₆N₄, 240.3; m/z found 241.1 [M+H]⁺.

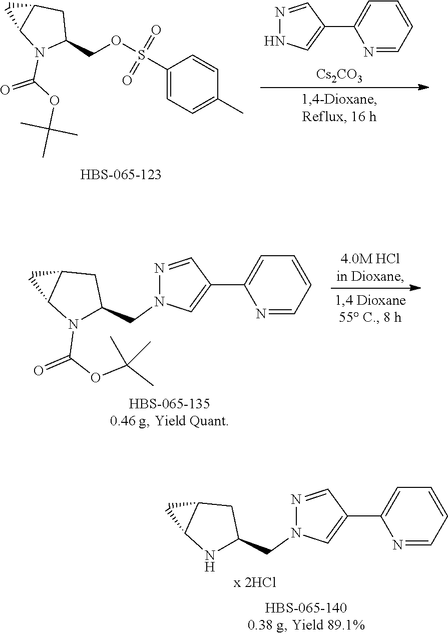

Step 1: Synthesis of HBS-065-135: Compound HBS-065-123 (0.5 g, 1.36 mmol) and 2-(1H-Pyrazol-4-yl)pyridine (0.22 g, 1.5 mmol) were dissolved in 1,4-Dioxane (10.0 mL). The anhydrous Cs₂CO₃ (0.89 g, 2.72 mmol) was added, and reaction mixture was stirred at reflux for 16 h. The LCMS shows product formation m/z 341.2. The rxn mixture was cooled at ambient temperature and diluted with water. The product was extracted with Ethyl acetate. The combined EtOAc layer was separated and dried over anhydrous Na₂SO₄. The evaporation of solvent gave crude product. The crude product was purified by column chromatography, Mobile phase: EtOAc:Hexane gradient. The 0.46 g of pure product was obtained (Yield Quant.). MS (ESI) mass calcd. for C₁₉H₂₄N₄O₂, 340.42; m/z found 341.2 [M+H]⁺.

Step 4: Synthesis of HBS-065-140: Compound HBS-065-135 (0.46 g, 1.36 mmol) was dissolved in Dry Dioxane (10.0 mL). The 4.0 M HCl solution in Dioxane (1.36 mL, 5.44 mmol) was added and rxn mixture was stirred at 55° C. temperature for 8 h. LCMS shows product formation m/z 241.2. The rxn mixture was cooled and ppts were filtered. The ppts were dried to obtain 0.38 g of solid product (Yield 89.1%). MS (ESI) mass calcd. for C₁₄H₁₆N₄, 240.3; m/z found 241.2 [M+H]⁺.

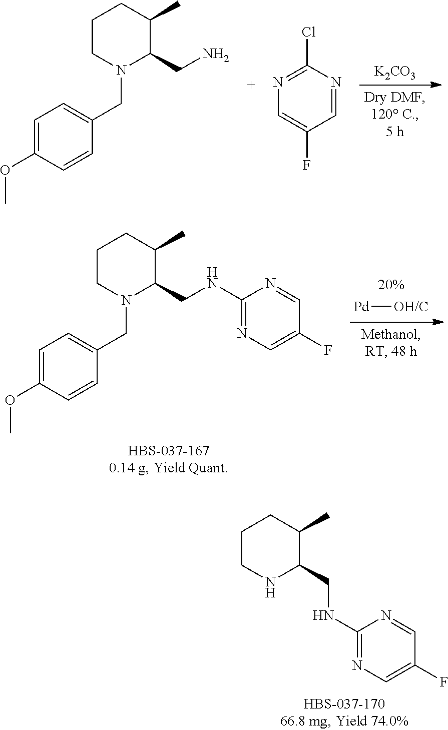

Step 1: Synthesis of HBS-037-167: [(2S,3R)-1-[4-methoxyphenyl)methyl]-3-methylpiperidine-2-yl]methanamine (0.1 g, 0.4 mmol) and 2-Chloro-5-Fluoro-pyrimidine (0.12 g, 0.81 mmol) were dissolved in Dry DMF (3.0 mL). The K₂CO₃ (0.14 g, 1.01 mmol) was added and rxn mixture was stirred at 120° C. temperature for 5 h. LCMS shows product formation m/z 345.2. The rxn mixture was diluted with water. The product was extracted with Ethyl acetate. The combined ethyl acetate layer was washed with water followed by brine. The organic layer was separated and dried over anhydrous sodium sulfate. The evaporation of solvent gave crude product. The crude product was purified by ISCO combi-flash chromatography system, Mobile phase: EtOAc:Hexane gradient. The 0.14 g of product was isolated (Yield Quant.). MS (ESI) mass calcd. for C₁₉H₂₅FN₄O, 344.43; m/z found 345.2 [M+H]⁺.

Step 2: Synthesis of HBS-037-170: Compound HBS-037-167 (0.14 g, 0.4 mmol) was dissolved in MeOH (4.0 mL). The 20.0% Pd—OH/C (28.0 mg) was added and rxn mixture was stirred at ambient temperature for 24 h. The LCMS shows unreacted starting material and product formation. The 20.0% Pd—OH/C (20.0 mg) was further added and rxn mixture was stirred at ambient temperature for another 24 h. The LCMS shows product formation m/z 225.1. The rxn mixture was filtered over celite and washed with MeOH. The filtrate was evaporated under reduced pressure to obtain 67.0 mg of crude product (Yield 74.0%). The crude product is used in the next step without purification. MS (ESI) mass calcd. for C₁₁H₁₇FN₄, 224.28; m/z found 225.1 [M+H]⁺.

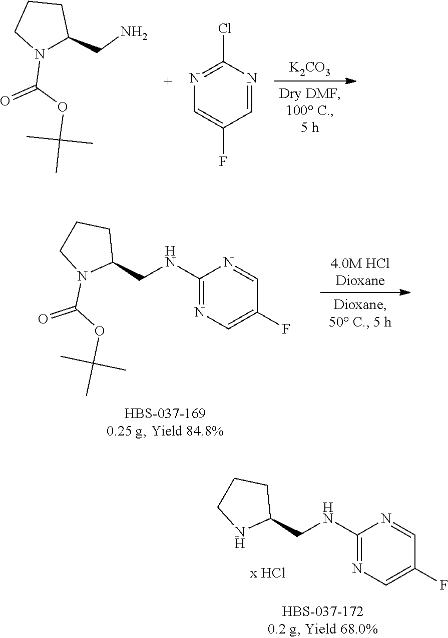

Step 1: Synthesis of HBS-037-169: (S)-1-Boc-2-(aminomethyl)-pyrrolidine (0.2 g, 0.1 mmol) and 2-Chloro-5-Fluoro-pyrimidine (0.2 g, 1.5 mmol) were dissolved in anhydrous DMF (4.0 mL). The K₂CO₃ (0.35 g, 2.5 mmol) was added, and the reaction mixture was heated to 100° C. temperature for 5 h. LCMS shows product formation m/z 297.0 and side product. The reaction mixture was cooled to ambient temperature and diluted with water. The product was extracted with Ethyl acetate. The combined ethyl acetate layer was washed with water followed by brine. The organic layer was separated and dried over anhydrous sodium sulfate. The evaporation of solvent gave crude product. The crude product was purified by ISCO combi-flash chromatography system, Mobile phase: EtOAc:Hexane gradient. The 0.25 g of major product was obtained (Yield 84.8%). MS (ESI) mass calcd. for C₁₄H₂₁FN₄O₂, 296.34; m/z found 297.0 [M+H]⁺.

Step 2: Synthesis of HBS-037-172: Compound HBS-037-169 (0.25 g, 0.85 mmol) was dissolved in anhydrous dioxane (3.0 mL). The 4.0 M HCl in dioxane (2.1 mL, 8.47 mmol) was added and the reaction was stirred at 50° C. temperature for 5 h. LCMS shows product formation m/z 197.0. The reaction mixture was cooled to ambient temperature and concentrated under reduced pressure to obtain 0.2 of product (Yield 68.0%). MS (ESI) mass calcd. for C₉H₁₃FN₄, 196.22; m/z found 197.0 [M+H]⁺.

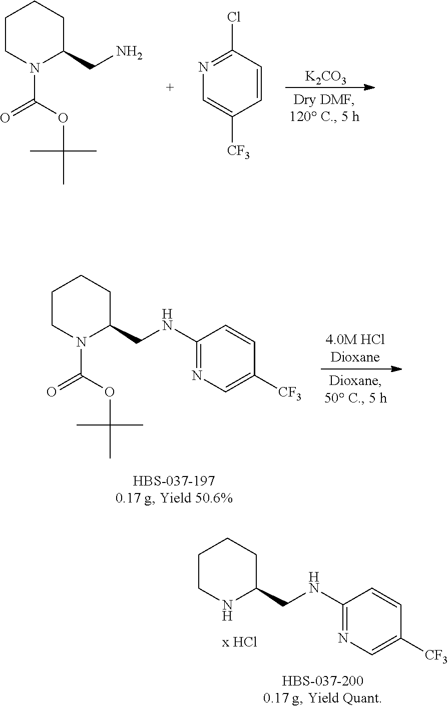

Step 1: Synthesis of HBS-037-197: Tert-butyl-(2S)-2-(aminomethyl)piperidin-1-carboxylate (0.2 g, 0.93 mmol) and 2-Chloro-5-trifluoromethylpyridine (0.2 g, 1.11 mmol) were dissolved in anhydrous DMF (5.0 mL). The K₂CO₃ (0.32 g, 2.32 mmol) was added, and the reaction mixture was heated to 120° C. temperature for 5 h. LCMS shows product formation m/z 360.0. The reaction mixture was cooled to ambient temperature and diluted with water. The product was extracted with Ethyl acetate. The combined ethyl acetate layer was washed with water followed by brine. The organic layer was separated and dried over anhydrous sodium sulfate. The evaporation of solvent gave crude product. The crude product was purified by ISCO combi-flash chromatography system, Mobile phase: EtOAc:Hexane gradient. The 0.17 g of product was obtained (Yield 50.6%). MS (ESI) mass calcd. for C₁₇H₂₄F₃N₃O₂, 359.39; m/z found 360.0 [M+H]⁺.

Step 2: Synthesis of HBS-037-200: Compound HBS-037-197 (0.17 g, 0.47 mmol) was dissolved in anhydrous dioxane (5.0 mL). The 4.0 M HCl in dioxane (1.2 mL, 4.7 mmol) was added and the reaction was stirred at 50° C. temperature for 5 h. LCMS shows product formation m/z 260.0. The reaction mixture was cooled to ambient temperature and concentrated under reduced pressure to obtain 0.17 of product (Yield Quant.). MS (ESI) mass calcd. for C₁₂H₁₆F₃N₃, 259.27; m/z found 260.0 [M+H]⁺.

### General Experimental Procedures for Synthesis of Example Compounds:

Method A: Acid Intermediate 1.0 eq. (0.1 mmol) was dissolved in anhydrous DCM (2.0 mL). The EDC·HCl 1.5 eq. (0.15 mmol) and HOBt 1.5 eq. (0.15 mmol) were added followed by Et₃N 5.0 eq. (0.5 mmol). The rxn mixture was stirred at ambient temperature for 5.0 min. Amine Intermediate (0.1 mmol) was added to the rxn mixture. The rxn mixture was stirred at ambient temperature for 16 h. The rxn mixture was diluted with DCM and washed with saturated solution of NaHCO₃. The DCM layer was separated and dried over anhydrous Na₂SO₄. The evaporation of solvent gave crude product. The crude product was purified by ISCO combi-flash system, Mobile phase: Ethyl Acetate:Hexane or DCM:MeOH gradient.

Method B: Acid Intermediate 1.0 eq. (0.1 mmol) and HATU 1.1 eq. (0.11 mmol) were dissolved in anhydrous DMF (1.5 mL). The DIPEA 4.0 eq. (0.4 mmol) was added and rxn mixture was stirred at ambient temperature for 5.0 min. Amine Intermediate (0.1 mmol) was added to the rxn mixture. The rxn mixture was stirred at ambient temperature for 16 h. The rxn mixture was diluted with DCM and washed with saturated solution of NaHCO₃. The DCM layer was separated and dried over anhydrous Na₂SO₄. The evaporation of solvent gave crude product. The crude product was purified by ISCO combi-flash system, Mobile phase: Ethyl Acetate:Hexane or DCM:MeOH gradient.

A summary of the acid intermediates, amine intermediates, and method used to produce each Example compound is shown in Table 2.

TABLE 2

| | Acid | Amine | | MW. | Found, |
|---|---|---|---|---|---|
| Example | Intermediate | Intermediate | Method | (g/mol) | [M + H]⁺ |
| 1 | HBS-037-054 | HBS-039-036 | B | 453.54 | 454.1 |
| 2 | HBS-037-054 | HBS-037-069 | B | 471.48 | 472.1 |
| 3 | HBS-037-054 | HBS-037-074 | B | 432.52 | 433.1 |
| 4 | HBS-037-163 | HBS-037-101 | A | 454.57 | 455.2 |
| 5 | HBS-037-054 | HBS-037-101 | A | 460.57 | 461.2 |
| 6 | HBS-037-163 | HBS-037-110 | A | 493.52 | 494.2 |
| 7 | HBS-037-054 | HBS-037-110 | A | 499.53 | 500.2 |
| 8 | HBS-037-193 | HBS-037-110 | A | 493.52 | 494.2 |
| 9 | HBS-037-193 | HBS-037-101 | A | 454.57 | 455.3 |
| 10 | HBS-037-163 | HBS-037-069 | A | 465.47 | 466.2 |
| 11 | HBS-037-193 | HBS-037-069 | A | 465.47 | 466.2 |
| 12 | HBS-037-193 | HBS-039-036 | A | 447.53 | 448.2 |
| 13 | HBS-037-163 | HBS-039-036 | A | 447.53 | 448.2 |
| 14 | HBS-037-193 | HBS-037-074 | A | 426.51 | 427.2 |
| 15 | HBS-037-163 | HBS-037-074 | A | 426.51 | 427.2 |
| 16 | HBS-037-054 | HBS-037-154 | A | 433.50 | 434.2 |
| 17 | HBS-037-054 | HBS-037-155 | A | 472.46 | 473.2 |
| 18 | HBS-037-193 | HBS-037-155 | A | 466.46 | 467.2 |
| 19 | HBS-037-193 | HBS-037-154 | A | 427.50 | 428.2 |
| 20 | HBS-037-163 | HBS-037-155 | A | 466.46 | 467.2 |
| 21 | HBS-037-163 | HBS-037-154 | A | 427.50 | 428.2 |
| 22 | HBS-037-163 | HBS-037-170 | A | 444.5 | 445.1 |
| 23 | HBS-037-163 | HBS-037-172 | A | 416.45 | 417.2 |
| 24 | HBS-037-163 | HBS-039-160 | A | 480.48 | 481.1 |
| 25 | HBS-037-163 | HBS-037-200 | A | 479.50 | 480.0 |
| 26 | HBS-037-193 | HBS-037-200 | A | 479.50 | 480.0 |
| 27 | HBS-037-054 | HBS-037-200 | A | 485.50 | 486.0 |
| 28 | HBS-039-018 | HBS-037-155 | A | 466.46 | 467.2 |
| 29 | HBS-039-018 | HBS-037-069 | A | 465.47 | 466.2 |
| 30 | HBS-037-163 | HBS-039-120 | A | 415.46 | 416.2 |
| 31 | HBS-039-132 | HBS-039-120 | A | 416.45 | 417.3 |
| 32 | HBS-039-132 | HBS-037-069 | A | 466.46 | 467.3 |
| 33 | HBS-037-193 | HBS-039-120 | A | 415.46 | 416.2 |
| 34 | HBS-039-132 | HBS-037-110 | A | 494.5 | 495.3 |
| 35 | HBS-039-132 | HBS-039-036 | A | 448.52 | 449.3 |
| 36 | HBS-039-139 | HBS-037-069 | A | 466.46 | 467.2 |
| 37 | HBS-037-163 | HBS-039-137 | A | 477.35 | 477.1 |
| 38 | HBS-037-193 | HBS-039-137 | A | 477.35 | 477.1 |
| 39 | HBS-039-139 | HBS-037-155 | A | 467.44 | 468.2 |
| 40 | HBS-039-150 | HBS-037-110 | A | 511.51 | 512.3 |
| 41 | HBS-039-150 | HBS-039-120 | A | 433.45 | 434.2 |
| 42 | HBS-039-150 | HBS-037-069 | A | 483.46 | 484.2 |
| 43 | HBS-039-150 | HBS-037-155 | A | 484.45 | 485.2 |
| 44 | HBS-039-150 | HBS-039-036 | A | 465.52 | 466.2 |
| 45 | HBS-037-193 | HBS-039-160 | A | 480.48 | 481.2 |
| 46 | HBS-037-054 | HBS-039-160 | A | 486.49 | 487.2 |
| 47 | HBS-039-132 | HBS-039-160 | A | 481.47 | 482.2 |
| 48 | HBS-039-132 | HBS-037-155 | A | 467.44 | 468.2 |
| 49 | HBS-054-014 | HBS-039-179 | A | 483.51 | 484.0 |
| 50 | HBS-054-021 | HBS-039-179 | A | 483.51 | 484.0 |
| 51 | HBS-054-014 | HBS-037-155 | A | 484.45 | 485.0 |
| 52 | HBS-054-021 | HBS-037-155 | A | 484.45 | 485.0 |
| 53 | HBS-054-080 | HBS-039-179 | A | 533.52 | 534.4 |
| 54 | HBS-054-088 | HBS-039-179 | A | 489.52 | 490.2 |
| 55 | HBS-039-150 | HBS-039-179 | A | 483.51 | 484.2 |
| 56 | HBS-039-150 | HBS-039-176 | A | 533.52 | 534.3 |
| 57 | HBS-037-054 | HBS-039-179 | A | 471.53 | 472.3 |
| 58 | HBS-037-054 | HBS-039-176 | A | 521.53 | 522.3 |
| 59 | HBS-037-163 | HBS-039-179 | A | 465.52 | 466.3 |
| 60 | HBS-037-163 | HBS-039-176 | A | 515.53 | 516.2 |
| 61 | HBS-054-015 | HBS-039-179 | A | 483.51 | 484.2 |
| 62 | HBS-054-016 | HBS-039-179 | A | 483.51 | 484.2 |
| 63 | HBS-039-192 | HBS-039-179 | A | 483.51 | 484.2 |
| 64 | HBS-039-192 | HBS-037-155 | A | 484.45 | 485.2 |
| 65 | HBS-054-016 | HBS-037-155 | A | 484.45 | 485.2 |
| 66 | HBS-055-013 | HBS-039-179 | A | 489.52 | 490.3 |
| 67 | HBS-039-192 | HBS-055-097 | A | 466.51 | 467.2 |
| 68 | HBS-039-192 | HBS-055-092 | A | 466.51 | 467.2 |
| 69 | HBS-039-192 | HBS-055-098 | A | 465.52 | 466.2 |
| 70 | HBS-055-013 | HBS-055-097 | A | 472.51 | 473.2 |
| 71 | HBS-055-013 | HBS-055-092 | A | 472.51 | 473.2 |
| 72 | HBS-055-013 | HBS-055-098 | A | 471.53 | 472.2 |
| 73 | HBS-054-014 | HBS-055-097 | A | 466.51 | 467.2 |
| 74 | HBS-054-014 | HBS-055-092 | A | 466.51 | 467.2 |
| 75 | HBS-054-014 | HBS-055-098 | A | 465.52 | 466.2 |
| 76 | HBS-037-054 | HBS-055-097 | A | 454.52 | 455.2 |
| 77 | HBS-037-054 | HBS-055-092 | A | 454.52 | 455.2 |
| 78 | HBS-054-080 | HBS-055-097 | A | 516.52 | 517.2 |
| 79 | HBS-054-080 | HBS-055-092 | A | 516.52 | 517.2 |
| 80 | HBS-054-080 | HBS-055-098 | A | 515.53 | 516.2 |
| 81 | HBS-054-080 | HBS-055-109 | A | 534.51 | 535.1 |
| 82 | HBS-039-192 | HBS-055-109 | A | 484.50 | 485.1 |
| 83 | HBS-055-012 | HBS-055-109 | A | 490.50 | 491.2 |
| 84 | HBS-054-014 | HBS-055-109 | A | 484.50 | 485.2 |
| 85 | HBS-037-054 | HBS-055-109 | A | 472.51 | 473.2 |
| 86 | HBS-037-054 | HBS-055-098 | A | 453.54 | 454.2 |
| 87 | HBS-037-054 | HBS-055-127 | A | 498.50 | 499.2 |
| 88 | HBS-037-163 | HBS-055-127 | A | 492.49 | 493.2 |
| 89 | HBS-037-193 | HBS-055-127 | A | 492.49 | 493.2 |
| 90 | HBS-039-192 | HBS-055-143 | A | 484.50 | 485.1 |
| 91 | HBS-055-013 | HBS-055-143 | A | 490.50 | 491.1 |
| 92 | HBS-054-014 | HBS-055-143 | A | 484.50 | 485.2 |
| 93 | HBS-037-054 | HBS-055-143 | A | 472.51 | 473.2 |
| 94 | HBS-054-080 | HBS-055-143 | A | 534.51 | 535.1 |
| 95 | HBS-039-192 | HBS-055-147 | A | 495.52 | 496.1 |
| 96 | HBS-055-013 | HBS-055-147 | A | 501.53 | 502.1 |
| 97 | HBS-054-014 | HBS-055-147 | A | 495.52 | 496.2 |
| 98 | HBS-054-080 | HBS-055-147 | A | 545.53 | 546.2 |
| 99 | HBS-039-192 | HBS-055-158 | A | 469.49 | 470.1 |
| 100 | HBS-055-013 | HBS-055-158 | A | 475.49 | 476.1 |
| 101 | HBS-054-014 | HBS-055-158 | A | 469.49 | 470.2 |
| 102 | HBS-054-080 | HBS-055-158 | A | 519.49 | 520.1 |
| 103 | HBS-039-192 | HBS-055-162 | A | 470.47 | 471.1 |
| 104 | HBS-055-013 | HBS-055-162 | A | 476.48 | 477.1 |
| 105 | HBS-054-014 | HBS-055-162 | A | 470.47 | 471.1 |
| 106 | HBS-054-080 | HBS-055-162 | A | 520.48 | 521.1 |
| 107 | HBS-039-192 | HBS-055-170 | A | 496.51 | 497.2 |
| 108 | HBS-055-013 | HBS-055-170 | A | 502.52 | 503.1 |
| 109 | HBS-054-014 | HBS-055-170 | A | 496.51 | 497.2 |
| 110 | HBS-054-080 | HBS-055-170 | A | 546.52 | 547.2 |
| 111 | HBS-039-018 | HBS-055-109 | A | 466.51 | 467.2 |
| 112 | HBS-039-018 | HBS-055-109 | A | 466.51 | 467.2 |
| 113 | HBS-037-054 | HBS-055-180 | A | 488.46 | 489.1 |
| 114 | HBS-037-163 | HBS-055-180 | A | 482.45 | 483.1 |
| 115 | HBS-039-018 | HBS-055-162 | A | 452.48 | 453.2 |
| 116 | HBS-037-054 | HBS-061-199 | A | 474.49 | 475.1 |
| 117 | HBS-039-018 | HBS-061-199 | A | 468.46 | 469.2 |
| 118 | HBS-039-019 | HBS-055-092 | A | 448.52 | 449.2 |
| 119 | HBS-039-019 | HBS-055-092 | A | 448.52 | 449.2 |
| 120 | HBS-037-163 | HBS-061-199 | A | 468.48 | 469.2 |
| 121 | HBS-054-014 | HBS-061-199 | A | 486.47 | 487.2 |
| 122 | HBS-039-019 | HBS-055-109 | A | 466.51 | 467.2 |
| 123 | HBS-039-019 | HBS-055-143 | A | 466.51 | 467.2 |
| 124 | HBS-039-019 | HBS-037-155 | A | 466.46 | 467.1 |
| 125 | HBS-039-019 | HBS-055-127 | A | 492.49 | 493.1 |
| 126 | HBS-039-019 | HBS-055-170 | A | 478.52 | 479.2 |
| 127 | HBS-039-019 | HBS-061-199 | A | 468.48 | 469.2 |
| 128 | HBS-039-019 | HBS-061-198 | A | 450.49 | 451.2 |
| 129 | HBS-062-013 | HBS-039-036 | A | 453.54 | 454.2 |
| 130 | HBS-055-197 | HBS-039-036 | A | 453.54 | 454.2 |
| 131 | HBS-062-028 | HBS-039-036 | A | 449.51 | 450.2 |
| 132 | HBS-062-030 | HBS-039-036 | A | 448.52 | 449.2 |
| 133 | HBS-062-027 | HBS-039-036 | A | 448.52 | 449.1 |
| 134 | HBS-062-013 | HBS-055-097 | A | 454.52 | 455.2 |
| 135 | HBS-055-197 | HBS-055-097 | A | 454.52 | 455.2 |
| 136 | HBS-062-041 | HBS-039-036 | A | 448.52 | 449.1 |
| 137 | HBS-062-041 | HBS-055-097 | A | 449.51 | 450.1 |
| 138 | HBS-062-028 | HBS-055-097 | A | 450.50 | 451.2 |
| 139 | HBS-062-030 | HBS-055-097 | A | 449.51 | 450.2 |
| 140 | HBS-062-027 | HBS-055-097 | A | 449.51 | 450.2 |
| 141 | HBS-062-013 | HBS-037-155 | A | 472.46 | 473.1 |
| 142 | HBS-055-197 | HBS-037-155 | A | 472.46 | 473.1 |
| 143 | HBS-039-018 | HBS-062-065 | A | 480.48 | 481.1 |
| 144 | HBS-039-019 | HBS-062-065 | A | 480.48 | 481.1 |
| 145 | HBS-062-013 | HBS-062-065 | A | 486.59 | 487.2 |
| 146 | HBS-062-041 | HBS-062-065 | A | 481.47 | 482.1 |
| 147 | HBS-062-030 | HBS-062-065 | B | 481.47 | 482.1 |
| 148 | HBS-062-028 | HBS-062-065 | A | 482.46 | 483.1 |
| 149 | HBS-039-019 | HBS-062-063 | A | 462.55 | 463.2 |
| 150 | HBS-039-018 | HBS-062-063 | A | 462.55 | 463.2 |
| 151 | HBS-037-163 | HBS-062-065 | A | 480.48 | 481.1 |
| 152 | HBS-062-013 | HBS-055-127 | A | 498.50 | 499.2 |
| 153 | HBS-062-028 | HBS-055-127 | A | 494.47 | 495.1 |
| 154 | HBS-062-013 | HBS-062-063 | A | 468.55 | 469.2 |
| 155 | HBS-062-028 | HBS-061-198 | A | 452.47 | 453.1 |
| 156 | HBS-062-030 | HBS-061-198 | B | 451.48 | 452.1 |
| 157 | HBS-055-197 | HBS-062-065 | A | 486.49 | 487.1 |
| 158 | HBS-062-027 | HBS-062-065 | A | 481.47 | 482.1 |
| 159 | HBS-062-013 | HBS-062-116 | A | 498.50 | 499.2 |
| 160 | HBS-039-019 | HBS-062-116 | A | 492.49 | 493.1 |
| 161 | HBS-062-028 | HBS-062-116 | A | 494.47 | 495.1 |
| 162 | HBS-062-013 | HBS-062-142 | A | 487.47 | 488.3 |
| 163 | HBS-039-019 | HBS-062-142 | A | 481.47 | 481.1 |
| 164 | HBS-062-030 | HBS-062-142 | B | 482.46 | 483.2 |
| 165 | HBS-055-197 | HBS-062-142 | A | 487.47 | 488.2 |
| 166 | HBS-062-028 | HBS-062-142 | A | 483.45 | 484.2 |
| 167 | HBS-062-041 | HBS-062-142 | A | 482.46 | 483.1 |
| 168 | HBS-062-027 | HBS-062-142 | A | 482.46 | 483.2 |
| 169 | HBS-037-054 | HBS-062-142 | A | 487.47 | 488.1 |
| 170 | HBS-039-018 | HBS-062-142 | A | 481.47 | 482.2 |
| 171 | HBS-062-013 | HBS-062-166 | A | 475.48 | 476.2 |
| 172 | HBS-062-182 | HBS-037-155 | A | 467.44 | 468.1 |
| 173 | HBS-062-182 | HBS-055-092 | A | 449.51 | 450.2 |
| 174 | HBS-062-182 | HBS-055-097 | B | 449.51 | 450.2 |
| 175 | HBS-062-182 | HBS-061-198 | B | 451.48 | 452.1 |
| 176 | HBS-062-182 | HBS-061-199 | B | 469.47 | 470.2 |
| 177 | HBS-054-014 | HBS-062-197 | A | 486.53 | 487.3 |
| 178 | HBS-062-028 | HBS-062-197 | A | 470.52 | 471.3 |
| 179 | HBS-065-004 | HBS-037-155 | A | 467.44 | 468.2 |
| 180 | HBS-065-004 | HBS-055-097 | B | 449.51 | 450.2 |
| 181 | HBS-065-004 | HBS-055-092 | B | 449.51 | 450.2 |
| 182 | HBS-062-182 | HBS-055-143 | B | 467.50 | 468.2 |
| 183 | HBS-062-182 | HBS-055-109 | B | 467.50 | 468.1 |
| 184 | HBS-065-025 | HBS-037-155 | B | 473.45 | 474.2 |
| 185 | HBS-062-186 | HBS-055-097 | B | 450.50 | 451.2 |
| 186 | HBS-062-186 | HBS-055-092 | B | 450.50 | 451.2 |
| 187 | HBS-062-186 | HBS-055-170 | B | 480.50 | 481.2 |
| 188 | HBS-062-186 | HBS-061-198 | B | 452.47 | 453.2 |
| 189 | HBS-062-186 | HBS-061-199 | B | 470.46 | 471.2 |
| 190 | HBS-062-186 | HBS-037-155 | B | 468.43 | 469.2 |
| 191 | HBS-062-028 | HBS-037-155 | B | 468.43 | 469.2 |
| 192 | HBS-062-030 | HBS-037-155 | B | 467.44 | 468.2 |
| 193 | HBS-062-186 | HBS-055-143 | B | 468.49 | 469.2 |
| 194 | HBS-062-186 | HBS-055-109 | B | 468.49 | 469.2 |
| 195 | HBS-062-192 | HBS-055-097 | B | 455.51 | 456.2 |
| 196 | HBS-062-192 | HBS-055-143 | B | 473.50 | 474.2 |
| 197 | HBS-062-192 | HBS-055-092 | B | 455.51 | 456.2 |
| 198 | HBS-062-192 | HBS-055-109 | B | 473.50 | 474.2 |
| 199 | HBS-062-192 | HBS-061-198 | B | 457.48 | 458.2 |
| 200 | HBS-062-192 | HBS-061-199 | B | 457.48 | 458.2 |
| 201 | HBS-062-192 | HBS-037-155 | B | 473.45 | 474.2 |
| 202 | HBS-062-192 | HBS-055-170 | B | 485.51 | 486.2 |
| 203 | HBS-062-028 | HBS-055-143 | B | 468.49 | 469.2 |
| 204 | HBS-062-028 | HBS-055-109 | B | 468.49 | 469.2 |
| 205 | HBS-062-030 | HBS-055-143 | B | 467.50 | 468.2 |
| 206 | HBS-062-030 | HBS-055-109 | B | 467.50 | 468.2 |
| 207 | HBS-065-004 | HBS-055-143 | B | 467.50 | 468.2 |
| 208 | HBS-065-004 | HBS-055-109 | B | 467.50 | 468.2 |
| 209 | HBS-065-004 | HBS-061-198 | B | 451.48 | 452.2 |
| 210 | HBS-065-004 | HBS-061-199 | B | 469.47 | 470.2 |
| 211 | HBS-065-019 | HBS-055-097 | B | 455.51 | 456.2 |
| 212 | HBS-065-019 | HBS-055-143 | B | 473.50 | 474.2 |
| 213 | HBS-065-019 | HBS-055-092 | B | 455.51 | 456.2 |
| 214 | HBS-065-019 | HBS-055-109 | B | 473.50 | 474.2 |
| 215 | HBS-062-025 | HBS-055-097 | B | 455.51 | 456.2 |
| 216 | HBS-062-025 | HBS-055-143 | B | 473.50 | 474.2 |
| 217 | HBS-062-025 | HBS-055-092 | B | 455.51 | 456.2 |
| 218 | HBS-062-025 | HBS-055-109 | B | 473.50 | 474.2 |
| 219 | HBS-062-025 | HBS-061-198 | B | 457.48 | 458.2 |
| 220 | HBS-062-025 | HBS-061-199 | B | 475.48 | 476.2 |
| 221 | HBS-065-019 | HBS-061-198 | B | 457.48 | 458.2 |
| 222 | HBS-065-019 | HBS-061-199 | B | 475.48 | 476.2 |
| 223 | HBS-065-019 | HBS-037-155 | B | 473.45 | 474.2 |
| 224 | HBS-065-019 | HBS-055-170 | B | 485.51 | 486.2 |
| 225 | HBS-065-004 | HBS-061-170 | B | 468.43 | 469.2 |
| 226 | HBS-037-193 | HBS-061-192 | A | 482.96 | 483.2 |
| 227 | HBS-037-193 | HBS-061-179 | A | 482.96 | 483.2 |
| 228 | HBS-062-182 | HBS-061-179 | B | 483.95 | 484.1 |
| 229 | HBS-062-013 | HBS-061-179 | B | 488.97 | 489.2 |
| 230 | HBS-055-197 | HBS-061-179 | B | 488.97 | 489.2 |
| 231 | HBS-039-019 | HBS-061-179 | A | 482.96 | 483.2 |
| 232 | HBS-062-013 | HBS-039-176 | A | 521.53 | 522.2 |
| 233 | HBS-055-197 | HBS-039-176 | A | 521.53 | 522.2 |
| 234 | HBS-062-013 | HBS-065-043 | A | 522.52 | 523.2 |
| 235 | HBS-055-197 | HBS-065-043 | A | 522.52 | 523.2 |
| 236 | HBS-065-055 | HBS-055-098 | A | 466.51 | 466.9 |
| 237 | HBS-065-055 | HBS-055-143 | B | 485.49 | 486.2 |
| 238 | HBS-065-055 | HBS-055-109 | B | 485.49 | 486.2 |
| 239 | HBS-065-064 | HBS-055-098 | B | 482.96 | 483.1 |
| 240 | HBS-065-064 | HBS-055-109 | B | 501.94 | 502.1 |
| 241 | HBS-065-064 | HBS-055-143 | B | 501.94 | 502.1 |
| 242 | HBS-065-076 | HBS-055-098 | B | 448.52 | 449.1 |
| 243 | HBS-065-076 | HBS-055-109 | B | 467.50 | 468.1 |
| 244 | HBS-065-076 | HBS-039-160 | B | 481.47 | 482.1 |
| 245 | HBS-065-013 | HBS-066-005 | A | 522.52 | 523.2 |
| 246 | HBS-065-076 | HBS-055-143 | B | 467.50 | 468.1 |
| 247 | HBS-065-013 | HBS-066-013 | A | 521.53 | 522.2 |
| 248 | HBS-055-197 | HBS-066-013 | A | 521.53 | 522.2 |
| 249 | HBS-055-197 | HBS-066-005 | A | 522.52 | 523.2 |
| 250 | HBS-065-127 | HBS-055-143 | A | 506.98 | 507.1 |
| 251 | HBS-065-127 | HBS-055-097 | A | 488.99 | 489.0 |
| 252 | HBS-065-127 | HBS-055-092 | A | 488.99 | 489.1 |
| 253 | HBS-065-127 | HBS-065-128 | A | 501.00 | 501.2 |
| 254 | HBS-037-054 | HBS-066-039 | A | 453.54 | 454.3 |
| 255 | HBS-037-054 | HBS-066-042 | A | 489.52 | 490.2 |
| 256 | HBS-037-054 | HBS-066-035 | A | 487.98 | 488.2 |
| 257 | HBS-037-193 | HBS-066-039 | A | 447.53 | 448.3 |
| 258 | HBS-039-018 | HBS-066-039 | A | 447.53 | 448.3 |
| 259 | HBS-065-159 | HBS-037-155 | B | 468.43 | 469.2 |
| 260 | HBS-065-159 | HBS-055-097 | B | 450.50 | 451.2 |
| 261 | HBS-065-159 | HBS-055-092 | B | 450.50 | 451.2 |
| 262 | HBS-065-127 | HBS-065-140 | A | 501.00 | 501.2 |
| 263 | HBS-065-159 | HBS-065-128 | B | 462.51 | 463.2 |

### II. Biological Assays Experimental

Antagonistic activities on both orexin receptors have been measured for each example compound using the following procedure:

### In Vitro Orexin Antagonism Measurement Assay: Intracellular Calcium Measurements:

Chinese hamster ovary (CHO) cells expressing the human orexin receptor and the human orexin-2 receptor, respectively, are grown in culture medium (Ham F-12 with L-Glutamine) containing 300 μg/mL G418, 100 U/mL penicillin, 100 μg/mL streptomycin and 10% heat inactivated fetal calf serum (FCS). The cells are seeded at 20,000 cells/well into 384-well black clear bottom sterile plates (Greiner). The seeded plates are incubated overnight at 37° C. in 5% 002. Human orexin-A as an agonist is prepared as 1 mM stock solution in MeOH: water (1:1), diluted in HBSS containing 0.1% bovine serum albumin (BSA), NaHCO₃: 0.375 g/L and 20 mM HEPES for use in the assay at a final concentration of 3 nM.

Antagonists are prepared as 10 mM stock solution in DMSO, then diluted in 384-well plates using DMSO followed by a transfer of the dilutions into in HBSS containing 0.1% bovine serum albumin (BSA), NaHCO₃: 0.375 g/L and 20 mM HEPES. On the day of the assay, 50 μL of staining buffer (HBSS containing 1% FCS, 20 mM HEPES, NaHCO₃: 0.375 g/L, 5 mM probenecid (Sigma) and 3 μM of the fluorescent calcium indicator fluo-4 AM (1 mM stock solution in DMSO, containing 10% pluronic) is added to each well. The 384-well cell-plates are incubated for 50 min at 37° C. in 5% CO₂ followed by equilibration at RT for 30 min before measurement.

Within the Fluorescent Imaging Plate Reader (FLIPR Tetra, Molecular Devices), antagonists are added to the plate in a volume of 10 μL/well, incubated for 120 min and finally 10 μL/well of agonist is added. Fluorescence is measured for each well at 1 second intervals, and the height of each fluorescence peak is compared to the height of the fluorescence peak induced by 3 nM orexin-A with vehicle in place of antagonist. The IC50 value (the concentration of compound needed to inhibit 50% of the agonistic response) is determined and may be normalized using the obtained IC50 value of an on-plate reference compound. Optimized conditions were achieved by adjustment of pipetting speed and cell splitting regime. The calculated IC₅₀values may fluctuate depending on the daily cellular assay performance. Fluctuations of this kind are known to those skilled in the art. In the case where IC₅₀values have been determined several times for the same compound, the geometric mean has been given. Antagonistic activities of example compounds are shown in Table 3.

Human k-Opioid (KOP) Receptor (Agonist Radioligand) Binding Assay

Purpose: Evaluation of the affinity of compounds for the human k-opioid receptor in transfected RBL cells determined in a radioligand binding assay.

Experimental protocol: Cell membrane homogenates (about 80 μg protein) are incubated for 60 min at 22° C. with 0.5 nM [3H]U-69593 in the absence or presence of the test compound in a buffer containing 50 mM Tris-HCl (pH 7.4), 10 mM MgCl2 and 1 mM EDTA. Nonspecific binding is determined in the presence of 10 μM naloxone. Following incubation, the samples are filtered rapidly under vacuum through glass fiber filters (CF/B, Packard) presoaked with 0.3% PEI and rinsed several times with ice-cold 50 mM Tris-HCl using a 96-sample cell harvester (Unifilter, Packard). The filters are dried then counted for radioactivity in a scintillation counter (Topcount, Packard) using a scintillation cocktail (Microscint 0, Packard). The results are expressed as a percent inhibition of the control radioligand specific binding. The standard reference compound is U-50488, which is tested in each experiment at several concentrations to obtain a competition curve from which its IC50 is calculated.

TABLE 3
Example of compounds reduced to practice with data (% Inhibition @ 1 uM), and selected compounds
with IC₅₀ values) at Orexin type 1 (OX₁R), Orexin type 2 (OX₂R) Receptors and
kappa-Opioid Receptors (kOR)
(“Ex.” Indicates Example Compound number)

| | | | | | | KOR | | |
|---|---|---|---|---|---|---|---|---|
| | | OX1R, | OX1R | OX2R, | OX2R | % | KOR | |
| | | % Inh. | IC50 | % | IC50 | Inh. | IC50 | MW. |
| Ex. | | @ | /Kb | Inh. @ | /Kb | @ | /Ki | (g/ |
| * | Structure | 1 uM | (nM) | 1 uM | (nM) | 1 uM | (nM) | mol) |
| 1 | 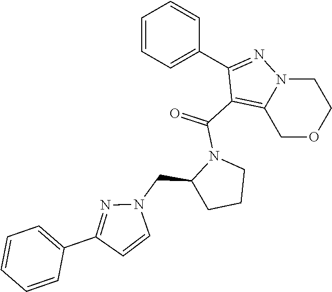 [SMILES: O=C(c1c(-c2ccccc2)nn2c1COCC2)N1CCC[C@H]1Cn1ccc(-c2ccccc2)n1] | 69.72 | 33/7 | 28.21 | | | | 453.54 |
| 2 | 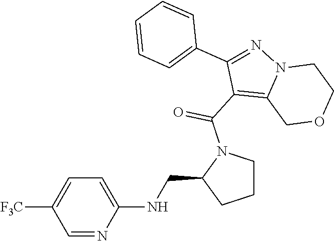 [SMILES: O=C(c1c(-c2ccccc2)nn2c1COCC2)N1CCC[C@H]1CNc1ccc(C(F)(F)F)cn1] | 62.84 | 120/26 | 20.90 | | | | 471.48 |
| 3 | 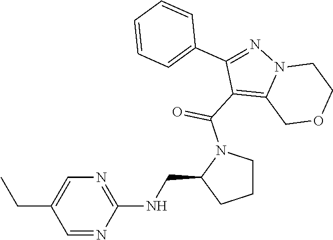 [SMILES: CCc1cnc(NC[C@@H]2CCCN2C(=O)c2c(-c3ccccc3)nn3c2COCC3)nc1] | 14.01 | | 14.24 | | | | 432.52 |
| 4 | 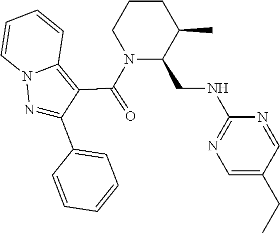 [SMILES: CCc1cnc(NC[C@@H]2[C@H](C)CCCN2C(=O)c2c(-c3ccccc3)nn3ccccc23)nc1] | 85.70 | 4/0.8 | 12.80 | | 52 | 1564/854 | 454.57 |
| 5 | 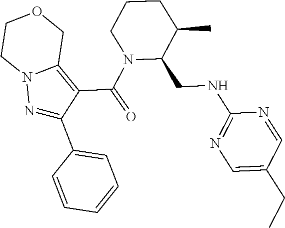 [SMILES: CCc1cnc(NC[C@@H]2[C@H](C)CCCN2C(=O)c2c(-c3ccccc3)nn3c2COCC3)nc1] | 70.97 | 140/29 | 0.4 | | | | 460.57 |
| 6 | 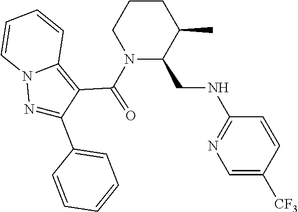 [SMILES: C[C@@H]1CCCN(C(=O)c2c(-c3ccccc3)nn3ccccc23)[C@@H]1CNc1ccc(C(F)(F)F)cn1] | 87.57 | 8/2 | 38.45 | | | | 493.52 |
| 7 | 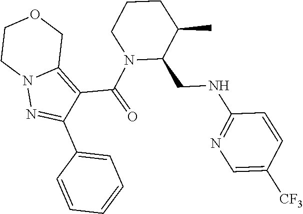 [SMILES: C[C@@H]1CCCN(C(=O)c2c(-c3ccccc3)nn3c2COCC3)[C@@H]1CNc1ccc(C(F)(F)F)cn1] | 80.10 | 9/2 | 2.44 | | 35 | 1465/799 | 499.53 |
| 8 | 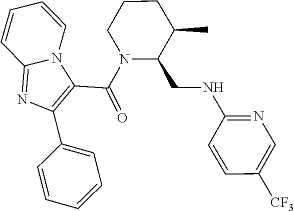 [SMILES: C[C@@H]1CCCN(C(=O)c2c(-c3ccccc3)nc3ccccn23)[C@@H]1CNc1ccc(C(F)(F)F)cn1] | 100 | 5/1 | 21.40 | | 38 | 1976/1078 | 493.52 |
| 9 | 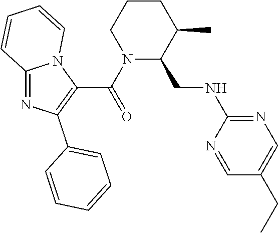 [SMILES: CCc1cnc(NC[C@@H]2[C@H](C)CCCN2C(=O)c2c(-c3ccccc3)nc3ccccn23)nc1] | 76.81 | 59/12 | −2.51 | | | | 454.57 |
| 10 | 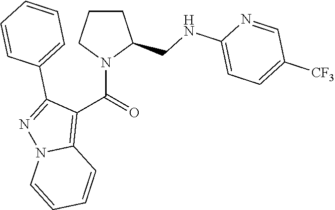 [SMILES: O=C(c1c(-c2ccccc2)nn2ccccc12)N1CCC[C@H]1CNc1ccc(C(F)(F)F)cn1] | 86.92 | 4/1 | 43.54 | | 26 | 6131/3344 | 465.47 |
| 11 | 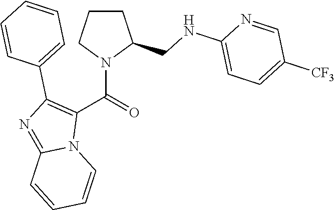 [SMILES: O=C(c1c(-c2ccccc2)nc2ccccn12)N1CCC[C@H]1CNc1ccc(C(F)(F)F)cn1] | 67.13 | 39/8 | 21.62 | | | | 465.47 |
| 12 | 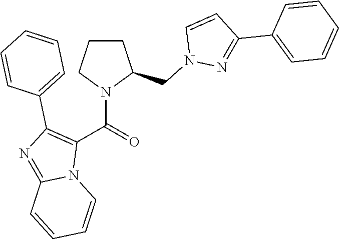 [SMILES: O=C(c1c(-c2ccccc2)nc2ccccn12)N1CCC[C@H]1Cn1ccc(-c2ccccc2)n1] | 88.90 | 2/0.5 | 46.14 | | | | 447.53 |
| 13 | 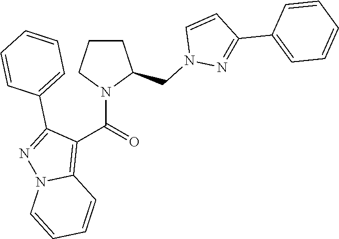 [SMILES: O=C(c1c(-c2ccccc2)nn2ccccc12)N1CCC[C@H]1Cn1ccc(-c2ccccc2)n1] | 96.60 | 3/0.6 | 98.47 | 70/10 | 56 | 1007/549 | 447.53 |
| 14 | 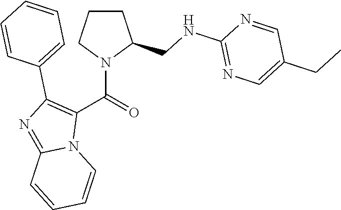 [SMILES: CCc1cnc(NC[C@@H]2CCCN2C(=O)c2c(-c3ccccc3)nc3ccccn23)nc1] | 14.79 | | 18.28 | | | | 426.51 |
| 15 | 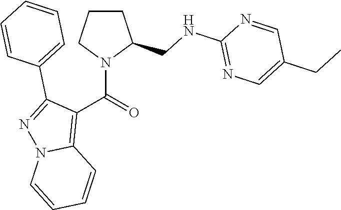 [SMILES: CCc1cnc(NC[C@@H]2CCCN2C(=O)c2c(-c3ccccc3)nn3ccccc23)nc1] | 54.08 | 180/38 | 37.30 | | | | 426.51 |
| 16 | 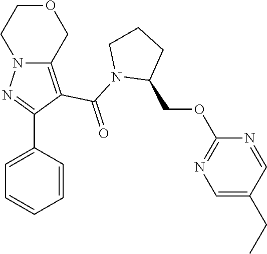 [SMILES: CCc1cnc(OC[C@@H]2CCCN2C(=O)c2c(-c3ccccc3)nn3c2COCC3)nc1] | 0.59 | | 7.77 | | | | 433.50 |
| 17 | 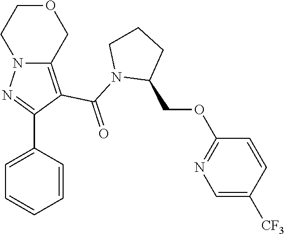 [SMILES: O=C(c1c(-c2ccccc2)nn2c1COCC2)N1CCC[C@H]1COc1ccc(C(F)(F)F)cn1] | 45.11 | | 5.78 | | | | 472.46 |
| 18 | 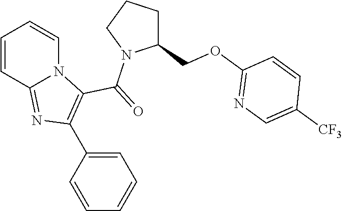 [SMILES: O=C(c1c(-c2ccccc2)nc2ccccn12)N1CCC[C@H]1COc1ccc(C(F)(F)F)cn1] | 69.98 | 290/62 | 29.61 | | | | 466.46 |
| 19 | 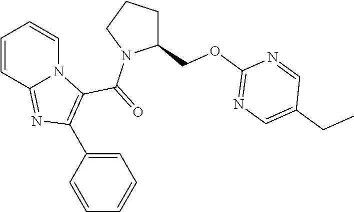 [SMILES: CCc1cnc(OC[C@@H]2CCCN2C(=O)c2c(-c3ccccc3)nc3ccccn23)nc1] | −10.79 | | 11.22 | | | | 427.50 |
| 20 | 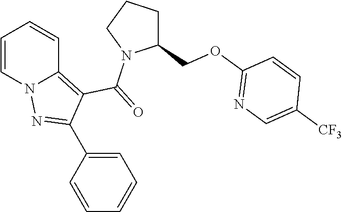 [SMILES: O=C(c1c(-c2ccccc2)nn2ccccc12)N1CCC[C@H]1COc1ccc(C(F)(F)F)cn1] | 84.89 | 11/2 | 49.58 | | 33 | 3497/1907 | 466.46 |
| 21 | 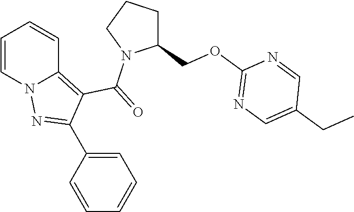 [SMILES: CCc1cnc(OC[C@@H]2CCCN2C(=O)c2c(-c3ccccc3)nn3ccccc23)nc1] | 38.75 | | 26.91 | | | | 427.50 |
| 22 | 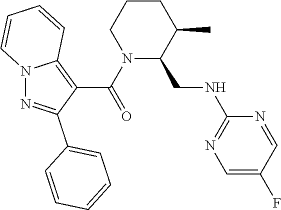 [SMILES: C[C@@H]1CCCN(C(=O)c2c(-c3ccccc3)nn3ccccc23)[C@@H]1CNc1ncc(F)cn1] | 82.2 | 10/2 | −3.0 | | | | 444.5 |
| 23 | 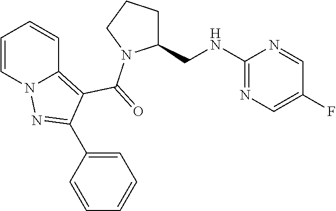 [SMILES: O=C(c1c(-c2ccccc2)nn2ccccc12)N1CCC[C@H]1CNc1ncc(F)cn1] | 23.0 | | 13.5 | | | | 416.45 |
| 24 | 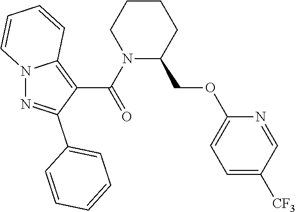 [SMILES: O=C(c1c(-c2ccccc2)nn2ccccc12)N1CCCC[C@H]1COc1ccc(C(F)(F)F)cn1] | 98.0 | 5/1 | 60.1 | | 52 | 1100/630 | 480.48 |
| 25 | 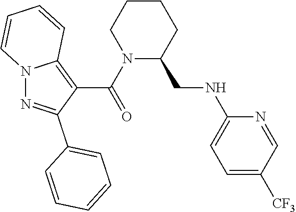 [SMILES: O=C(c1c(-c2ccccc2)nn2ccccc12)N1CCCC[C@H]1CNc1ccc(C(F)(F)F)cn1] | 99.8 | 17/4 | 72.0 | 74/10 | | | 479.50 |
| 26 | 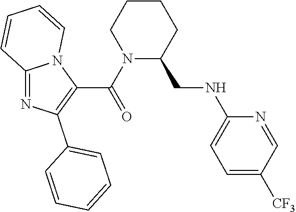 [SMILES: O=C(c1c(-c2ccccc2)nc2ccccn12)N1CCCC[C@H]1CNc1ccc(C(F)(F)F)cn1] | 99.5 | 14/3 | 39.4 | | | | 479.50 |
| 27 | 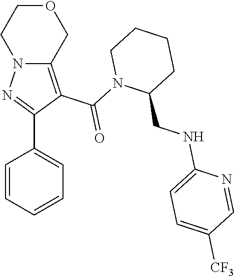 [SMILES: O=C(c1c(-c2ccccc2)nn2c1COCC2)N1CCCC[C@H]1CNc1ccc(C(F)(F)F)cn1] | 99.0 | 9/2 | 50.4 | | | | 485.50 |
| 28 | 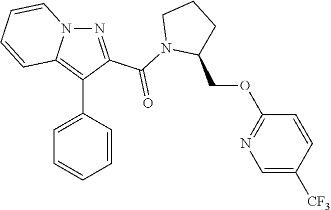 [SMILES: O=C(c1nn2ccccc2c1-c1ccccc1)N1CCC[C@H]1COc1ccc(C(F)(F)F)cn1] | 98.4 | 4/1 | 52.5 | | | | 466.46 |
| 29 | 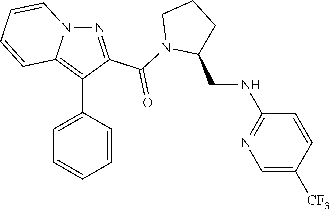 [SMILES: O=C(c1nn2ccccc2c1-c1ccccc1)N1CCC[C@H]1CNc1ccc(C(F)(F)F)cn1] | 99.5 | 5/1 | 84.1 | 35/5 | | | 465.47 |
| 30 | 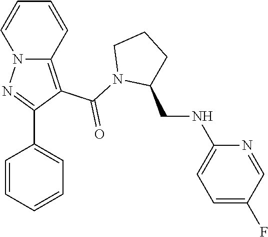 [SMILES: O=C(c1c(-c2ccccc2)nn2ccccc12)N1CCC[C@H]1CNc1ccc(F)cn1] | 32.5 | | 14.8 | | | | 415.46 |
| 31 | 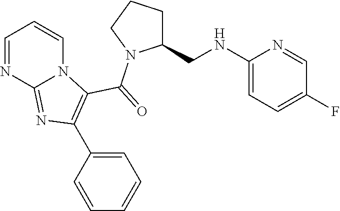 [SMILES: O=C(c1c(-c2ccccc2)nc2ncccn12)N1CCC[C@H]1CNc1ccc(F)cn1] | 4.7 | | −3.4 | | | | 416.45 |
| 32 | 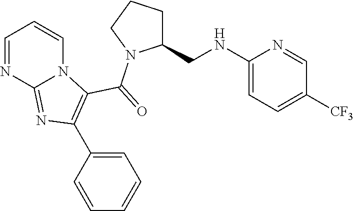 [SMILES: O=C(c1c(-c2ccccc2)nc2ncccn12)N1CCC[C@H]1CNc1ccc(C(F)(F)F)cn1] | 19.7 | | 3.6 | | | | 466.46 |
| 33 | 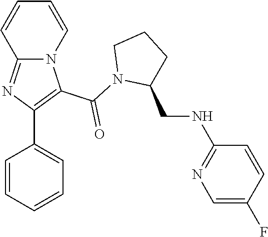 [SMILES: O=C(c1c(-c2ccccc2)nc2ccccn12)N1CCC[C@H]1CNc1ccc(F)cn1] | 4.8 | | 0.7 | | | | 415.46 |
| 34 | 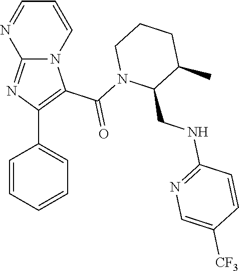 [SMILES: C[C@@H]1CCCN(C(=O)c2c(-c3ccccc3)nc3ncccn23)[C@@H]1CNc1ccc(C(F)(F)F)cn1] | 79.8 | 53/11 | 1.8 | | 57 | 730/400 | 494.5 |
| 35 | 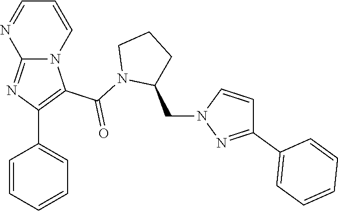 [SMILES: O=C(c1c(-c2ccccc2)nc2ncccn12)N1CCC[C@H]1Cn1ccc(-c2ccccc2)n1] | 54.8 | | 23.7 | | | | 448.52 |
| 36 | 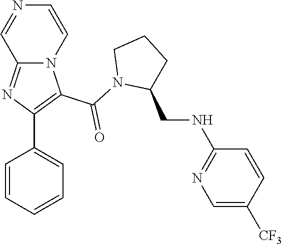 [SMILES: O=C(c1c(-c2ccccc2)nc2cnccn12)N1CCC[C@H]1CNc1ccc(C(F)(F)F)cn1] | 53.7 | | 14.7 | | | | 466.46 |
| 37 | 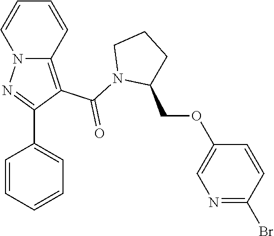 [SMILES: O=C(c1c(-c2ccccc2)nn2ccccc12)N1CCC[C@H]1COc1ccc(Br)nc1] | 25.4 | | 10.4 | | | | 477.35 |
| 38 | 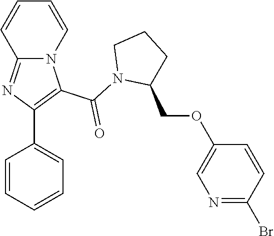 [SMILES: O=C(c1c(-c2ccccc2)nc2ccccn12)N1CCC[C@H]1COc1ccc(Br)nc1] | 8.2 | | −6.1 | | | | 477.35 |
| 39 | 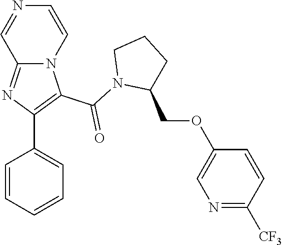 [SMILES: O=C(c1c(-c2ccccc2)nc2cnccn12)N1CCC[C@H]1COc1ccc(C(F)(F)F)nc1] | 39.4 | | 7.9 | | | | 467.44 |
| 40 | 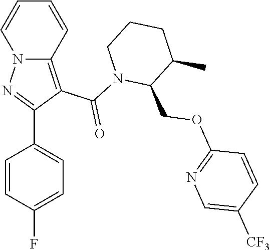 [SMILES: C[C@@H]1CCCN(C(=O)c2c(-c3ccc(F)cc3)nn3ccccc23)[C@@H]1COc1ccc(C(F)(F)F)cn1] | 98.4 | 29/6 | 7.1 | | 59 | 740/400 | 511.51 |
| 41 | 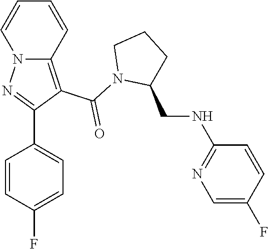 [SMILES: O=C(c1c(-c2ccc(F)cc2)nn2ccccc12)N1CCC[C@H]1CNc1ccc(F)cn1] | 45.6 | | 3.2 | | | | 433.45 |
| 42 | 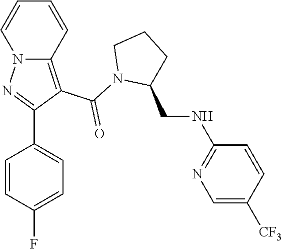 [SMILES: O=C(c1c(-c2ccc(F)cc2)nn2ccccc12)N1CCC[C@H]1CNc1ccc(C(F)(F)F)cn1] | 98.2 | 12/3 | 39.4 | | | | 483.46 |
| 43 | 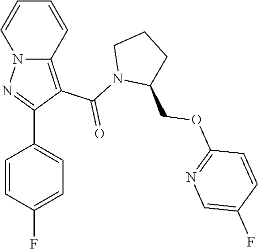 [SMILES: O=C(c1c(-c2ccc(F)cc2)nn2ccccc12)N1CCC[C@H]1COc1ccc(F)cn1] | 93.1 | 9/2 | 28.3 | | | | 484.45 |
| 44 | 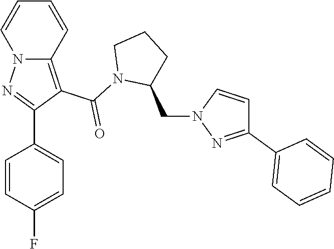 [SMILES: O=C(c1c(-c2ccc(F)cc2)nn2ccccc12)N1CCC[C@H]1Cn1ccc(-c2ccccc2)n1] | 98.8 | 5/1 | 72.3 | 220/31 | 55 | 690/370 | 465.52 |
| 45 | 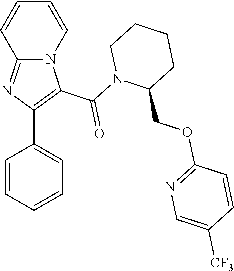 [SMILES: O=C(c1c(-c2ccccc2)nc2ccccn12)N1CCCC[C@H]1COc1ccc(C(F)(F)F)cn1] | 98.7 | 4/0.9 | 38.8 | | | | 480.48 |
| 46 | 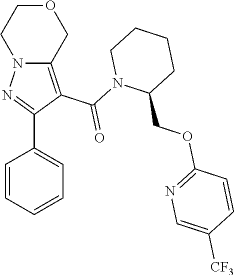 [SMILES: O=C(c1c(-c2ccccc2)nn2c1COCC2)N1CCCC[C@H]1COc1ccc(C(F)(F)F)cn1] | 94.8 | 7/1 | 25.3 | | 38 | | 486.49 |
| 47 | 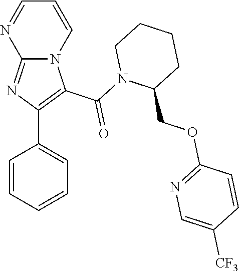 [SMILES: O=C(c1c(-c2ccccc2)nc2ncccn12)N1CCCC[C@H]1COc1ccc(C(F)(F)F)cn1] | 59.5 | | 1.2 | | | | 481.47 |
| 48 | 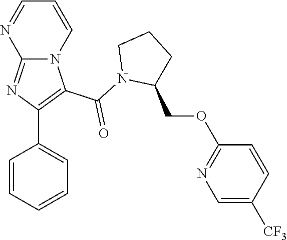 [SMILES: O=C(c1c(-c2ccccc2)nc2ncccn12)N1CCC[C@H]1COc1ccc(C(F)(F)F)cn1] | 18.9 | | −1.1 | | | | 467.44 |
| 49 | 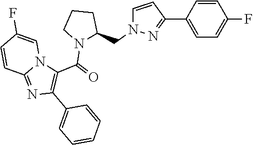 [SMILES: O=C(c1c(-c2ccccc2)nc2ccc(F)cn12)N1CCC[C@H]1Cn1ccc(-c2ccc(F)cc2)n1] | 101.98 | 9/2 | 79.34 | 52/7 | 92 | 47/25 | 483.51 |
| 50 | 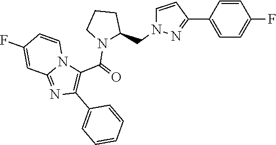 [SMILES: O=C(c1c(-c2ccccc2)nc2cc(F)ccn12)N1CCC[C@H]1Cn1ccc(-c2ccc(F)cc2)n1] | 99 | 3/0.6 | 89 | 18/3 | 59 | 590/320 | 483.51 |
| 51 | 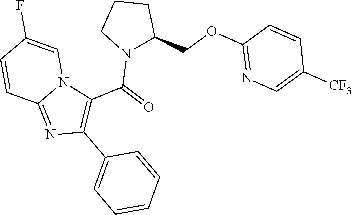 [SMILES: O=C(c1c(-c2ccccc2)nc2ccc(F)cn12)N1CCC[C@H]1COc1ccc(C(F)(F)F)cn1] | 88.10 | 80/17 | 46.55 | | | | 484.45 |
| 52 | 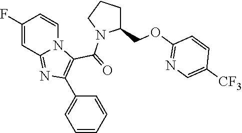 [SMILES: O=C(c1c(-c2ccccc2)nc2cc(F)ccn12)N1CCC[C@H]1COc1ccc(C(F)(F)F)cn1] | 79.21 | 327/69 | 43.26 | | | | 484.45 |
| 53 | 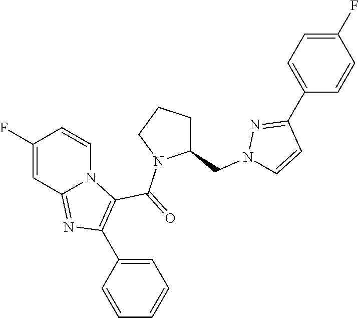 [SMILES: O=C(c1c(-c2ccccc2)nc2cc(F)ccn12)N1CCC[C@H]1Cn1ccc(-c2ccc(F)cc2)n1] | 97.63 | 21/4 | 95.54 | 33/5 | 93 | 56/31 | 533.52 |
| 54 | 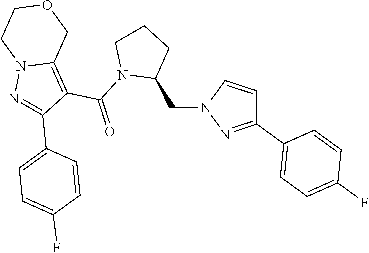 [SMILES: O=C(c1c(-c2ccc(F)cc2)nn2c1COCC2)N1CCC[C@H]1Cn1ccc(-c2ccc(F)cc2)n1] | 98 | 122/26 | 45.06 | | | | 489.52 |
| 55 | 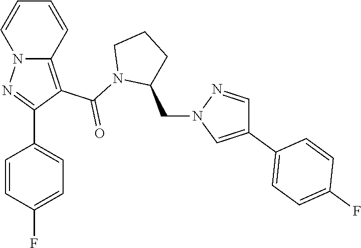 [SMILES: O=C(c1c(-c2ccc(F)cc2)nn2ccccc12)N1CCC[C@H]1Cn1cc(-c2ccc(F)cc2)cn1] | 100 | 5/1 | 93 | 33/5 | 66 | 480/260 | 483.51 |
| 56 | 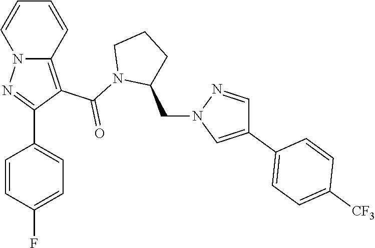 [SMILES: O=C(c1c(-c2ccc(F)cc2)nn2ccccc12)N1CCC[C@H]1Cn1cc(-c2ccc(C(F)(F)F)cc2)cn1] | 59 | 576/121 | 12 | | 76 | 450/250 | 533.52 |
| 57 | 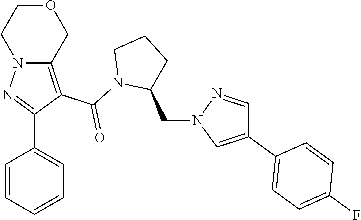 [SMILES: O=C(c1c(-c2ccccc2)nn2c1COCC2)N1CCC[C@H]1Cn1cc(-c2ccc(F)cc2)cn1] | 73 | 65/14 | 52 | 773/107 | 76 | 440/240 | 471.53 |
| 58 | 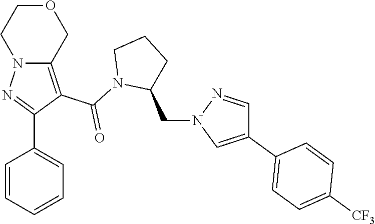 [SMILES: O=C(c1c(-c2ccccc2)nn2c1COCC2)N1CCC[C@H]1Cn1cc(-c2ccc(C(F)(F)F)cc2)cn1] | 9.22 | | 6.67 | | 85 | 130/69 | 521.53 |
| 59 | 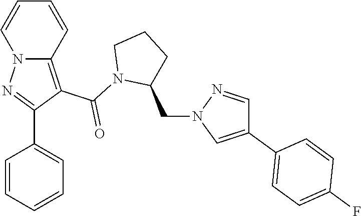 [SMILES: O=C(c1c(-c2ccccc2)nn2ccccc12)N1CCC[C@H]1Cn1cc(-c2ccc(F)cc2)cn1] | 91 | 4/0.7 | 99 | 11/2 | 56 | 470/260 | 465.52 |
| 60 | 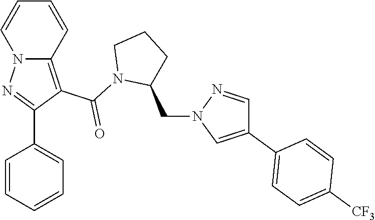 [SMILES: O=C(c1c(-c2ccccc2)nn2ccccc12)N1CCC[C@H]1Cn1cc(-c2ccc(C(F)(F)F)cc2)cn1] | 78 | 106/22 | 37 | | 63 | 390/210 | 515.53 |
| 61 | 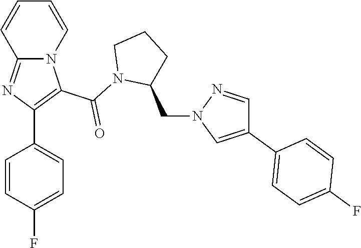 [SMILES: O=C(c1c(-c2ccc(F)cc2)nc2ccccn12)N1CCC[C@H]1Cn1cc(-c2ccc(F)cc2)cn1] | 85 | 29/6 | 73 | 76/10 | 74 | 280/150 | 483.51 |
| 62 | 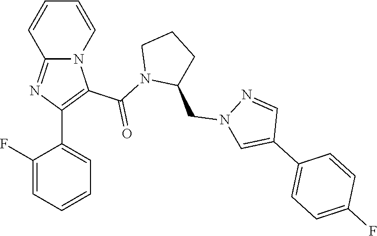 [SMILES: O=C(c1c(-c2ccccc2F)nc2ccccn12)N1CCC[C@H]1Cn1cc(-c2ccc(F)cc2)cn1] | 96.96 | 6/1 | 85.23 | 26/4 | 79 | 200/110 | 483.51 |
| 63 | 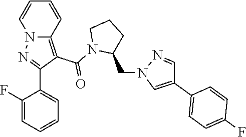 [SMILES: O=C(c1c(-c2ccccc2F)nn2ccccc12)N1CCC[C@H]1Cn1cc(-c2ccc(F)cc2)cn1] | 98 | 4/0.8 | 100 | 6/0.9 | 75 | 280150 | 483.51 |
| 64 | 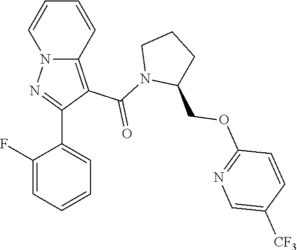 [SMILES: O=C(c1c(-c2ccccc2F)nn2ccccc12)N1CCC[C@H]1COc1ccc(C(F)(F)F)cn1] | 81 | 11/2 | 67 | 275/38 | 37 | | 484.45 |
| 65 | 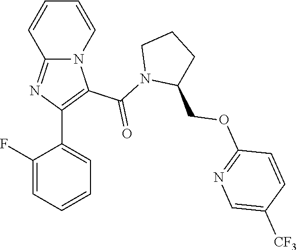 [SMILES: O=C(c1c(-c2ccccc2F)nc2ccccn12)N1CCC[C@H]1COc1ccc(C(F)(F)F)cn1] | 64 | 53/11 | 41 | | 11 | | 484.45 |
| 66 | 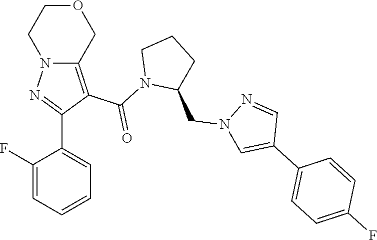 [SMILES: O=C(c1c(-c2ccccc2F)nn2c1COCC2)N1CCC[C@H]1Cn1cc(-c2ccc(F)cc2)cn1] | 84 | 33/7 | 68 | 131/18 | 77 | 220/120 | 489.52 |
| 67 | 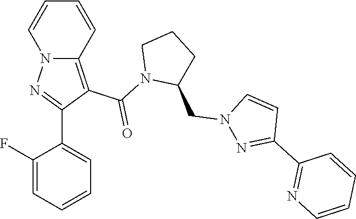 [SMILES: O=C(c1c(-c2ccccc2F)nn2ccccc12)N1CCC[C@H]1Cn1ccc(-c2ccccn2)n1] | 100 | 13/3 | 74 | 20/4 | 27 | | 466.51 |
| 68 | 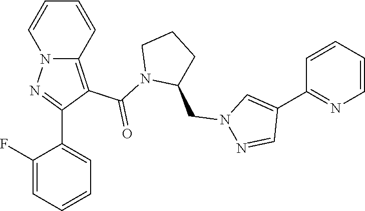 [SMILES: O=C(c1c(-c2ccccc2F)nn2ccccc12)N1CCC[C@H]1Cn1cc(-c2ccccn2)cn1] | 82 | 47/10 | 64 | 170/30 | 48 | | 466.51 |
| 69 | 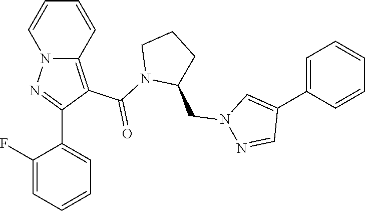 [SMILES: O=C(c1c(-c2ccccc2F)nn2ccccc12)N1CCC[C@H]1Cn1cc(-c2ccccc2)cn1] | 100 | 9/2 | 86 | 23/4 | 76 | 190/100 | 465.52 |
| 70 | 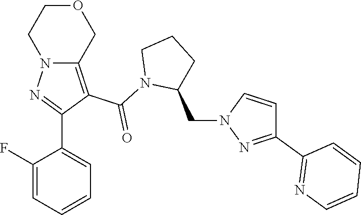 [SMILES: O=C(c1c(-c2ccccc2F)nn2c1COCC2)N1CCC[C@H]1Cn1ccc(-c2ccccn2)n1] | 60 | 120/25 | 35 | | | | 472.51 |
| 71 | 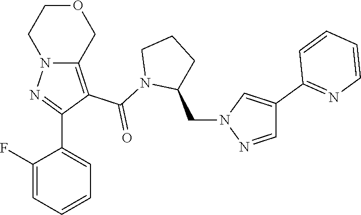 [SMILES: O=C(c1c(-c2ccccc2F)nn2c1COCC2)N1CCC[C@H]1Cn1cc(-c2ccccn2)cn1] | 14 | | 40 | | | | 472.51 |
| 72 | 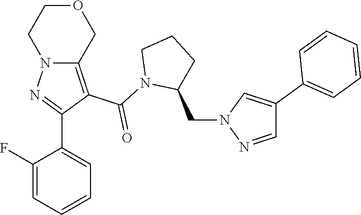 [SMILES: O=C(c1c(-c2ccccc2F)nn2c1COCC2)N1CCC[C@H]1Cn1cc(-c2ccccc2)cn1] | 64 | 620/13 | 45 | | | | 471.53 |
| 73 | 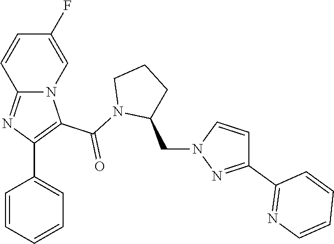 [SMILES: O=C(c1c(-c2ccccc2)nc2ccc(F)cn12)N1CCC[C@H]1Cn1ccc(-c2ccccn2)n1] | 82 | 9/2 | 48 | | 37 | | 466.51 |
| 74 | 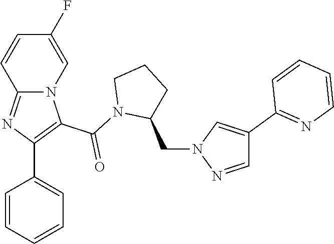 [SMILES: O=C(c1c(-c2ccccc2)nc2ccc(F)cn12)N1CCC[C@H]1Cn1cc(-c2ccccn2)cn1] | 40 | | 45 | | | | 466.51 |
| 75 | 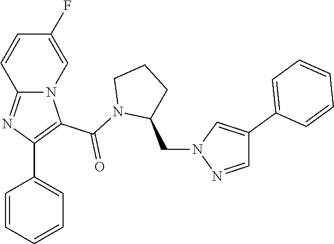 [SMILES: O=C(c1c(-c2ccccc2)nc2ccc(F)cn12)N1CCC[C@H]1Cn1cc(-c2ccccc2)cn1] | 93 | 160/33 | 76 | 160/28 | 86 | 140/77 | 465.52 |
| 76 | 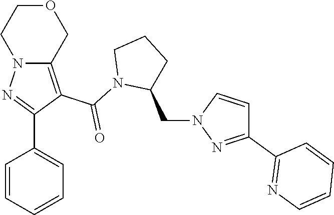 [SMILES: O=C(c1c(-c2ccccc2)nn2c1COCC2)N1CCC[C@H]1Cn1ccc(-c2ccccn2)n1] | 46 | | 25 | | | | 454.52 |
| 77 | 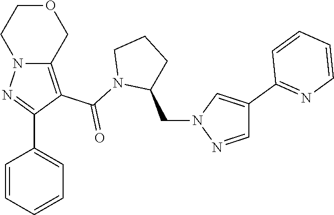 [SMILES: O=C(c1c(-c2ccccc2)nn2c1COCC2)N1CCC[C@H]1Cn1cc(-c2ccccn2)cn1] | 22 | | 23 | | | | 454.52 |
| 78 | 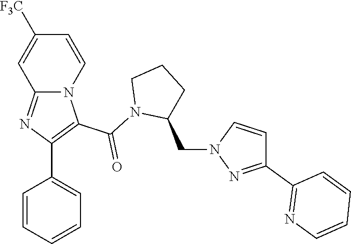 [SMILES: O=C(c1c(-c2ccccc2)nc2cc(C(F)(F)F)ccn12)N1CCC[C@H]1Cn1ccc(-c2ccccn2)n1] | 78 | 160/34 | 55 | 270/48 | 52 | | 516.52 |
| 79 | 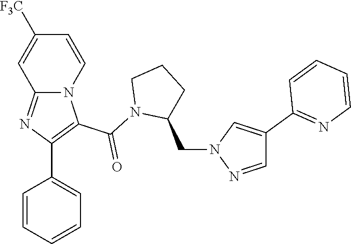 [SMILES: O=C(c1c(-c2ccccc2)nc2cc(C(F)(F)F)ccn12)N1CCC[C@H]1Cn1cc(-c2ccccn2)cn1] | 43 | | 62 | 77/13 | | | 516.52 |
| 80 | 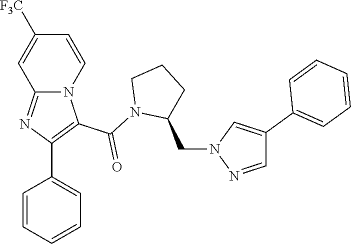 [SMILES: O=C(c1c(-c2ccccc2)nc2cc(C(F)(F)F)ccn12)N1CCC[C@H]1Cn1cc(-c2ccccc2)cn1] | 87 | 190/40 | 78 | 79/14 | 95 | 46/25 | 515.53 |
| 81 | 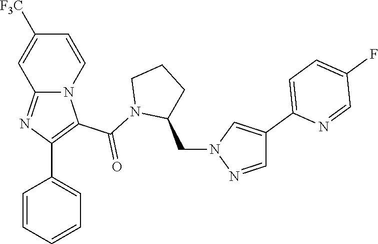 [SMILES: O=C(c1c(-c2ccccc2)nc2cc(C(F)(F)F)ccn12)N1CCC[C@H]1Cn1cc(-c2ccc(F)cn2)cn1] | 58 | 400/84 | 63 | 70/12 | | | 534.51 |
| 82 | 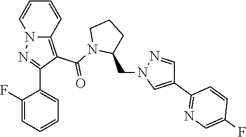 [SMILES: O=C(c1c(-c2ccccc2F)nn2ccccc12)N1CCC[C@H]1Cn1cc(-c2ccc(F)cn2)cn1] | 85 | 220/10 | 74 | 46/10 | 33 | | 484.50 |
| 83 | 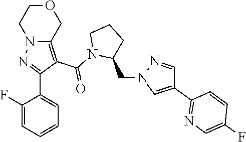 [SMILES: O=C(c1c(-c2ccccc2F)nn2c1COCC2)N1CCC[C@H]1Cn1cc(-c2ccc(F)cn2)cn1] | 17 | | 39 | | | | 490.50 |
| 84 | 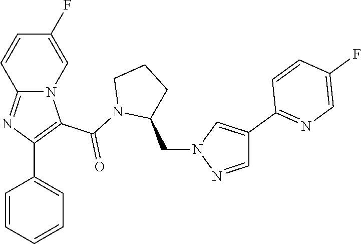 [SMILES: O=C(c1c(-c2ccccc2)nc2ccc(F)cn12)N1CCC[C@H]1Cn1cc(-c2ccc(F)cn2)cn1] | 32 | | 58 | 400/70 | | | 484.50 |
| 85 | 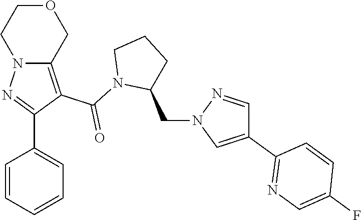 [SMILES: O=C(c1c(-c2ccccc2)nn2c1COCC2)N1CCC[C@H]1Cn1cc(-c2ccc(F)cn2)cn1] | 19 | | 35 | | | | 472.51 |
| 86 | 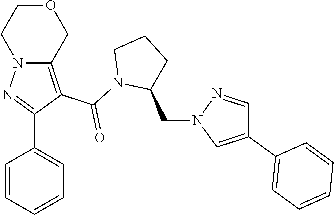 [SMILES: O=C(c1c(-c2ccccc2)nn2c1COCC2)N1CCC[C@H]1Cn1cc(-c2ccccc2)cn1] | 56 | 1300/280 | 42 | | | | 453.54 |
| 87 | 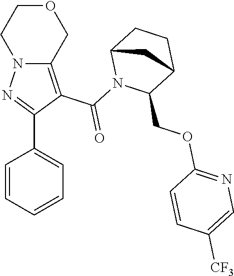 [SMILES: O=C(c1c(-c2ccccc2)nn2c1COCC2)N1[C@@H]2CC[C@@H](C2)[C@H]1COc1ccc(C(F)(F)F)cn1] | 78 | 230/49 | 23 | | 3 | | 498.50 |
| 88 | 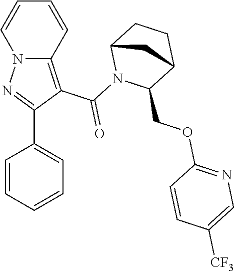 [SMILES: O=C(c1c(-c2ccccc2)nn2ccccc12)N1[C@@H]2CC[C@@H](C2)[C@H]1COc1ccc(C(F)(F)F)cn1] | 97 | 37/8 | 66 | 360/63 | 22 | | 492.49 |
| 89 | 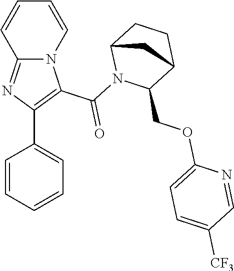 [SMILES: O=C(c1c(-c2ccccc2)nc2ccccn12)N1[C@@H]2CC[C@@H](C2)[C@H]1COc1ccc(C(F)(F)F)cn1] | 98 | 80/2 | 46 | | 11 | | 492.49 |
| 90 | 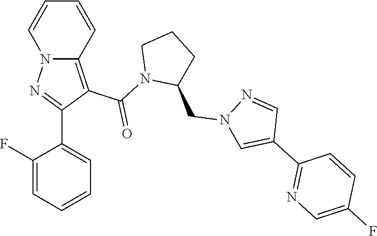 [SMILES: O=C(c1c(-c2ccccc2F)nn2ccccc12)N1CCC[C@H]1Cn1cc(-c2ccc(F)cn2)cn1] | 99 | 6/1 | 84 | 15/3 | 33 | | 484.50 |
| 91 | 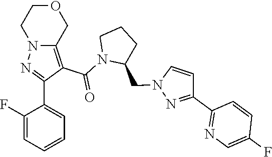 [SMILES: O=C(c1c(-c2ccccc2F)nn2c1COCC2)N1CCC[C@H]1Cn1ccc(-c2ccc(F)cn2)n1] | 58 | 540/110 | 38 | | | | 490.50 |
| 92 | 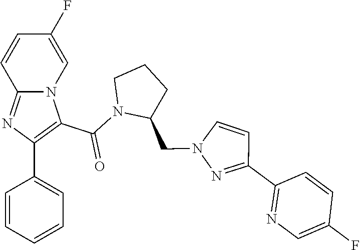 [SMILES: O=C(c1c(-c2ccccc2)nc2ccc(F)cn12)N1CCC[C@H]1Cn1ccc(-c2ccc(F)cn2)n1] | 75 | 250/52 | 61 | 930/160 | 79 | 170/94 | 484.50 |
| 93 | 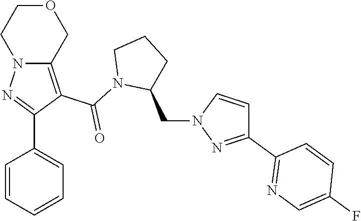 [SMILES: O=C(c1c(-c2ccccc2)nn2c1COCC2)N1CCC[C@H]1Cn1ccc(-c2ccc(F)cn2)n1] | 41 | | 36 | | | | 472.51 |
| 94 | 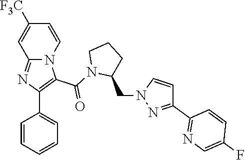 [SMILES: O=C(c1c(-c2ccccc2)nc2cc(C(F)(F)F)ccn12)N1CCC[C@H]1Cn1ccc(-c2ccc(F)cn2)n1] | 88 | 37/8 | 76 | 36/6 | 74 | 210/120 | 534.51 |
| 95 | 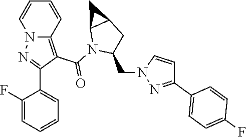 [SMILES: O=C(c1c(-c2ccccc2F)nn2ccccc12)N1[C@H](Cn2ccc(-c3ccc(F)cc3)n2)C[C@@H]2C[C@@H]21] | 91 | 95/20 | 50 | | 87 | 180/99 | 495.52 |
| 96 | 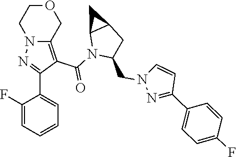 [SMILES: O=C(c1c(-c2ccccc2F)nn2c1COCC2)N1[C@H](Cn2ccc(-c3ccc(F)cc3)n2)C[C@@H]2C[C@@H]21] | 44 | | 22 | | | | 501.53 |
| 97 | 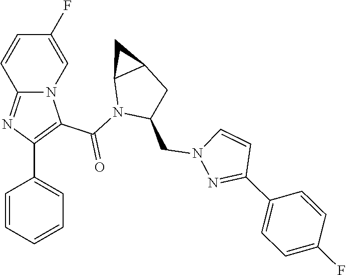 [SMILES: O=C(c1c(-c2ccccc2)nc2ccc(F)cn12)N1[C@H](Cn2ccc(-c3ccc(F)cc3)n2)C[C@@H]2C[C@@H]21] | 70 | 160/33 | 37 | | 91 | 80/44 | 495.52 |
| 98 | 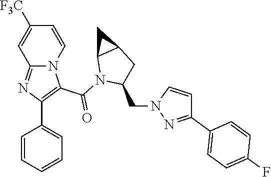 [SMILES: O=C(c1c(-c2ccccc2)nc2cc(C(F)(F)F)ccn12)N1[C@H](Cn2ccc(-c3ccc(F)cc3)n2)C[C@@H]2C[C@@H]21] | 48 | | 35 | | | | 545.53 |
| 99 | 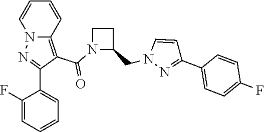 [SMILES: O=C(c1c(-c2ccccc2F)nn2ccccc12)N1CC[C@H]1Cn1ccc(-c2ccc(F)cc2)n1] | 59 | 440/92 | 61 | 270/46 | | | 469.49 |
| 100 | 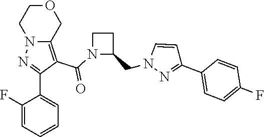 [SMILES: O=C(c1c(-c2ccccc2F)nn2c1COCC2)N1CC[C@H]1Cn1ccc(-c2ccc(F)cc2)n1] | −2 | | 19 | | | | 475.49 |
| 101 | 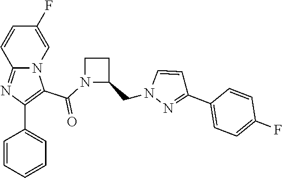 [SMILES: O=C(c1c(-c2ccccc2)nc2ccc(F)cn12)N1CC[C@H]1Cn1ccc(-c2ccc(F)cc2)n1] | 25 | | 47 | | | | 469.49 |
| 102 | 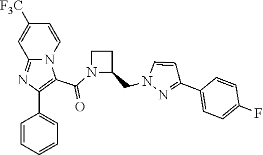 [SMILES: O=C(c1c(-c2ccccc2)nc2cc(C(F)(F)F)ccn12)N1CC[C@H]1Cn1ccc(-c2ccc(F)cc2)n1] | 31 | | 60 | 660/110 | | | 519.49 |
| 103 | 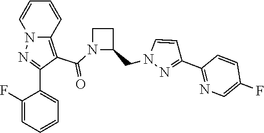 [SMILES: O=C(c1c(-c2ccccc2F)nn2ccccc12)N1CC[C@H]1Cn1ccc(-c2ccc(F)cn2)n1] | 24 | | 43 | | | | 470.47 |
| 104 | 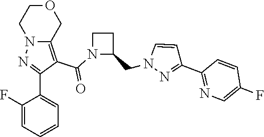 [SMILES: O=C(c1c(-c2ccccc2F)nn2c1COCC2)N1CC[C@H]1Cn1ccc(-c2ccc(F)cn2)n1] | 8 | | 14 | | | | 476.48 |
| 105 | 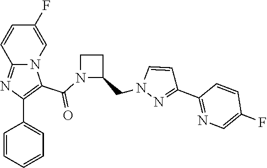 [SMILES: O=C(c1c(-c2ccccc2)nc2ccc(F)cn12)N1CC[C@H]1Cn1ccc(-c2ccc(F)cn2)n1] | 5 | | 29 | | | | 470.47 |
| 106 | 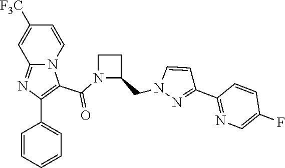 [SMILES: O=C(c1c(-c2ccccc2)nc2cc(C(F)(F)F)ccn12)N1CC[C@H]1Cn1ccc(-c2ccc(F)cn2)n1] | 19 | | 39 | | | | 520.48 |
| 107 | 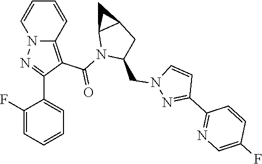 [SMILES: O=C(c1c(-c2ccccc2F)nn2ccccc12)N1[C@H](Cn2ccc(-c3ccc(F)cn3)n2)C[C@@H]2C[C@@H]21] | 92 | 83/18 | 55 | 520/90 | 61 | 910/500 | 496.51 |
| 108 | 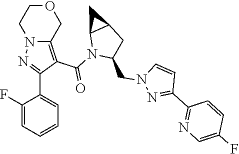 [SMILES: O=C(c1c(-c2ccccc2F)nn2c1COCC2)N1[C@H](Cn2ccc(-c3ccc(F)cn3)n2)C[C@@H]2C[C@@H]21] | 23 | | 17 | | | | 502.52 |
| 109 | 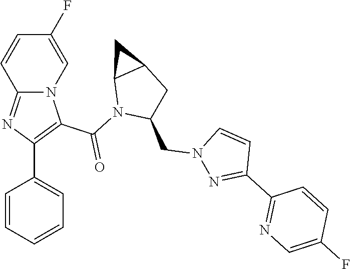 [SMILES: O=C(c1c(-c2ccccc2)nc2ccc(F)cn12)N1[C@H](Cn2ccc(-c3ccc(F)cn3)n2)C[C@@H]2C[C@@H]21] | 45 | | 32 | | | | 496.51 |
| 110 | 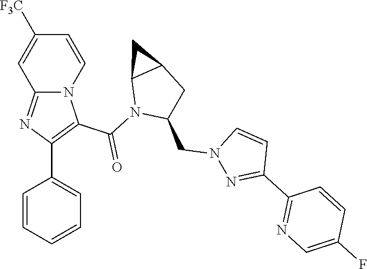 [SMILES: O=C(c1c(-c2ccccc2)nc2cc(C(F)(F)F)ccn12)N1[C@H](Cn2ccc(-c3ccc(F)cn3)n2)C[C@@H]2C[C@@H]21] | 69 | 280/60 | 42 | | | | 546.52 |
| 111 | 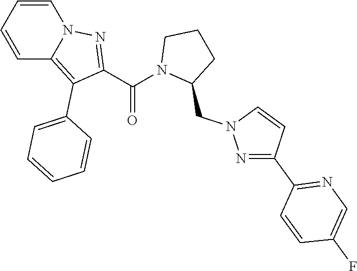 [SMILES: O=C(c1nn2ccccc2c1-c1ccccc1)N1CCC[C@H]1Cn1ccc(-c2ccc(F)cn2)n1] | 100 | 14/3 | 79 | 43/8 | 21 | | 466.51 |
| 112 | 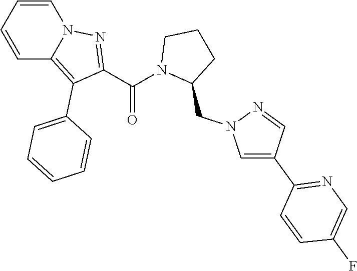 [SMILES: O=C(c1nn2ccccc2c1-c1ccccc1)N1CCC[C@H]1Cn1cc(-c2ccc(F)cn2)cn1] | 100 | 4/0.8 | 86 | 15/3 | 70 | 720/390 | 466.51 |
| 113 | 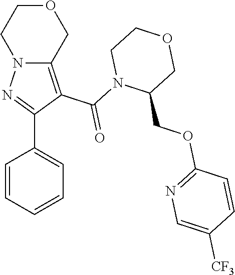 [SMILES: O=C(c1c(-c2ccccc2)nn2c1COCC2)N1CCOC[C@H]1COc1ccc(C(F)(F)F)cn1] | 12 | | 8 | | | | 488.46 |
| 114 | 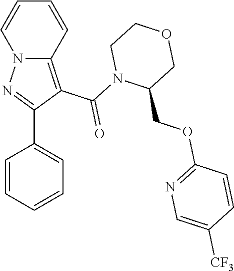 [SMILES: O=C(c1c(-c2ccccc2)nn2ccccc12)N1CCOC[C@H]1COc1ccc(C(F)(F)F)cn1] | 14 | | 6 | | | | 482.45 |
| 115 | 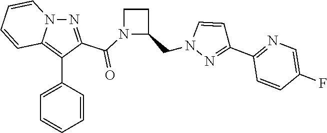 [SMILES: O=C(c1nn2ccccc2c1-c1ccccc1)N1CC[C@H]1Cn1ccc(-c2ccc(F)cn2)n1] | 60 | 650/140 | 38 | | 51 | | 452.48 |
| 116 | 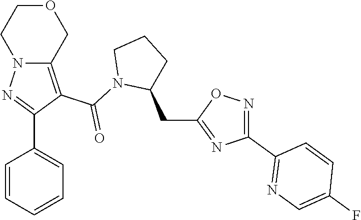 [SMILES: O=C(c1c(-c2ccccc2)nn2c1COCC2)N1CCC[C@H]1Cc1nc(-c2ccc(F)cn2)no1] | 10 | | 19 | | | | 474.49 |
| 117 | 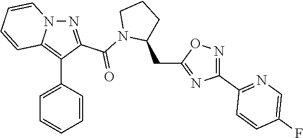 [SMILES: O=C(c1nn2ccccc2c1-c1ccccc1)N1CCC[C@H]1Cc1nc(-c2ccc(F)cn2)no1] | 100 | 2/0.3 | 69 | 150/26 | 19 | | 468.46 |
| 118 | 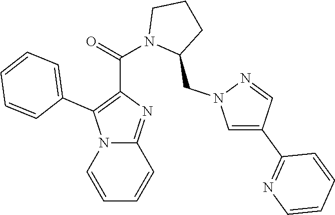 [SMILES: O=C(c1nc2ccccn2c1-c1ccccc1)N1CCC[C@H]1Cn1cc(-c2ccccn2)cn1] | 100 | 5/1 | 45 | | 35 | | 448.52 |
| 119 | 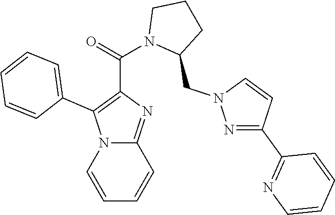 [SMILES: O=C(c1nc2ccccn2c1-c1ccccc1)N1CCC[C@H]1Cn1ccc(-c2ccccn2)n1] | 100 | 5/1 | 37 | | 6 | | 448.52 |
| 120 | 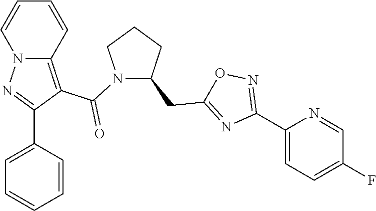 [SMILES: O=C(c1c(-c2ccccc2)nn2ccccc12)N1CCC[C@H]1Cc1nc(-c2ccc(F)cn2)no1] | 80 | 46/10 | 63 | 300/52 | 22 | | 468.48 |
| 121 | 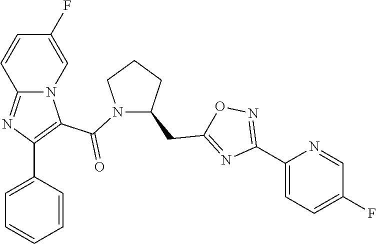 [SMILES: O=C(c1c(-c2ccccc2)nc2ccc(F)cn12)N1CCC[C@H]1Cc1nc(-c2ccc(F)cn2)no1] | 44 | | 45 | | | | 486.47 |
| 122 | 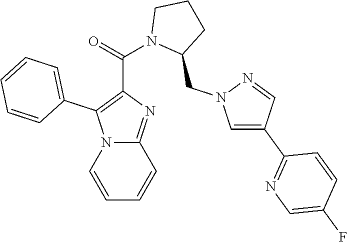 [SMILES: O=C(c1nc2ccccn2c1-c1ccccc1)N1CCC[C@H]1Cn1cc(-c2ccc(F)cn2)cn1] | 99 | 10/2 | 51 | | 15 | | 466.51 |
| 123 | 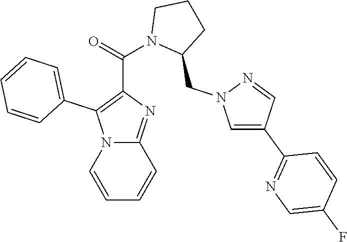 [SMILES: O=C(c1nc2ccccn2c1-c1ccccc1)N1CCC[C@H]1Cn1cc(-c2ccc(F)cn2)cn1] | 100 | 14/3 | 42 | | | | 466.51 |
| 124 | 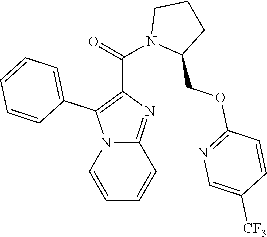 [SMILES: O=C(c1nc2ccccn2c1-c1ccccc1)N1CCC[C@H]1COc1ccc(C(F)(F)F)cn1] | 100 | 5/1 | 36 | | | | 466.46 |
| 125 | 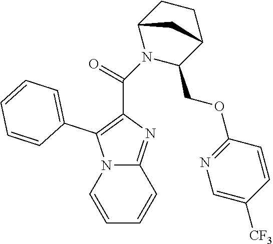 [SMILES: O=C(c1nc2ccccn2c1-c1ccccc1)N1[C@@H]2CC[C@@H](C2)[C@H]1COc1ccc(C(F)(F)F)cn1] | 93 | 190/41 | 57 | 20/3 | 7 | | 492.49 |
| 126 | 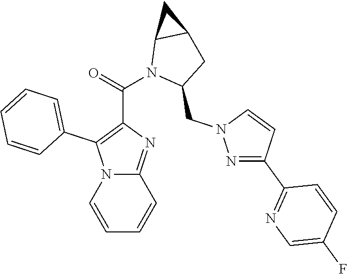 [SMILES: O=C(c1nc2ccccn2c1-c1ccccc1)N1[C@H](Cn2ccc(-c3ccc(F)cn3)n2)C[C@@H]2C[C@@H]21] | 99 | 17/4 | 24 | | 10 | | 478.52 |
| 127 | 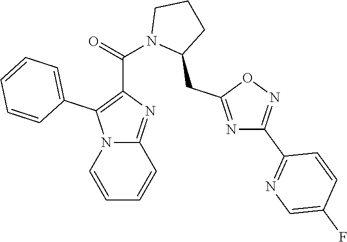 [SMILES: O=C(c1nc2ccccn2c1-c1ccccc1)N1CCC[C@H]1Cc1nc(-c2ccc(F)cn2)no1] | 100 | 5/1 | 31 | | 17 | | 468.48 |
| 128 | 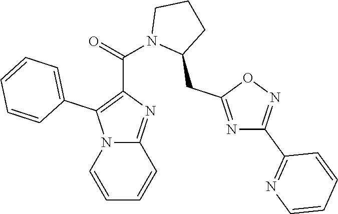 [SMILES: O=C(c1nc2ccccn2c1-c1ccccc1)N1CCC[C@H]1Cc1nc(-c2ccccn2)no1] | 100 | 7/1 | 17 | | 19 | | 450.49 |
| 129 | 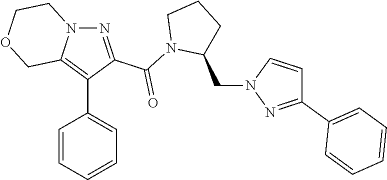 [SMILES: O=C(c1nn2c(c1-c1ccccc1)COCC2)N1CCC[C@H]1Cn1ccc(-c2ccccc2)n1] | 100 | 6/1 | 88 | 240/41 | 7 | | 453.54 |
| 130 | 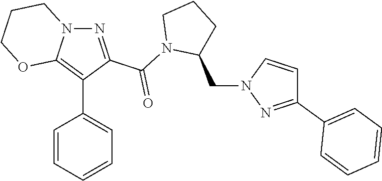 [SMILES: O=C(c1nn2c(c1-c1ccccc1)OCCC2)N1CCC[C@H]1Cn1ccc(-c2ccccc2)n1] | 100 | 9/2 | 98 | 110/19 | 53 | | 453.54 |
| 131 | 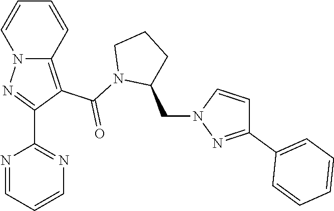 [SMILES: O=C(c1c(-c2ncccn2)nn2ccccc12)N1CCC[C@H]1Cn1ccc(-c2ccccc2)n1] | 100 | 4/1 | 91 | 68/12 | 27 | | 449.51 |
| 132 | 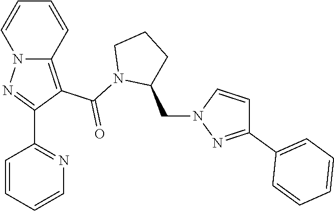 [SMILES: O=C(c1c(-c2ccccn2)nn2ccccc12)N1CCC[C@H]1Cn1ccc(-c2ccccc2)n1] | 100 | 4/1 | 98 | 16/3 | 35 | | 448.52 |
| 133 | 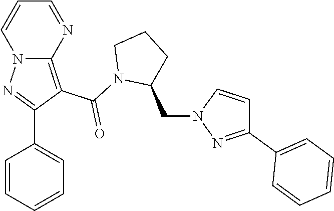 [SMILES: O=C(c1c(-c2ccccc2)nn2cccnc12)N1CCC[C@H]1Cn1ccc(-c2ccccc2)n1] | 99 | 4/1 | 81 | 83/14 | 44 | | 448.52 |
| 134 | 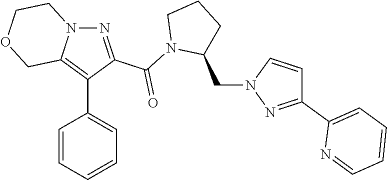 [SMILES: O=C(c1nn2c(c1-c1ccccc1)COCC2)N1CCC[C@H]1Cn1ccc(-c2ccccn2)n1] | 100 | 12/3 | 44 | | 0 | | 454.52 |
| 135 | 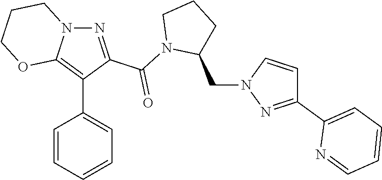 [SMILES: O=C(c1nn2c(c1-c1ccccc1)OCCC2)N1CCC[C@H]1Cn1ccc(-c2ccccn2)n1] | 100 | 4/1 | 71 | 660/110 | 22 | | 454.52 |
| 136 | 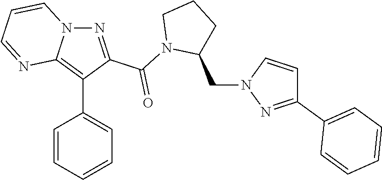 [SMILES: O=C(c1nn2cccnc2c1-c1ccccc1)N1CCC[C@H]1Cn1ccc(-c2ccccc2)n1] | 99 | 9/1 | 74 | 3/0.5 | 26 | | 448.52 |
| 137 | 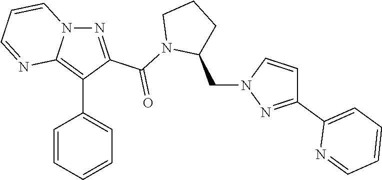 [SMILES: O=C(c1nn2cccnc2c1-c1ccccc1)N1CCC[C@H]1Cn1ccc(-c2ccccn2)n1] | 99 | 5/1 | 52 | 490/85 | 9 | | 449.51 |
| 138 | 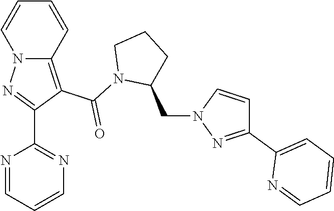 [SMILES: O=C(c1c(-c2ncccn2)nn2ccccc12)N1CCC[C@H]1Cn1ccc(-c2ccccn2)n1] | 100 | 9/2 | 62 | 490/84 | 0 | | 450.50 |
| 139 | 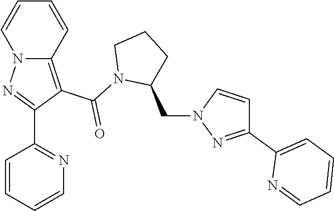 [SMILES: O=C(c1c(-c2ccccn2)nn2ccccc12)N1CCC[C@H]1Cn1ccc(-c2ccccn2)n1] | 100 | 8/2 | 82 | 63/11 | 0 | | 449.51 |
| 140 | 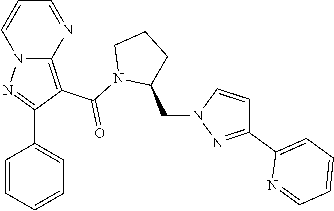 [SMILES: O=C(c1c(-c2ccccc2)nn2cccnc12)N1CCC[C@H]1Cn1ccc(-c2ccccn2)n1] | 100 | 4/1 | 59 | 190/32 | 14 | | 449.51 |
| 141 | 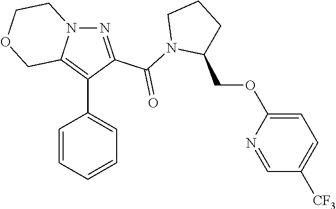 [SMILES: O=C(c1nn2c(c1-c1ccccc1)COCC2)N1CCC[C@H]1COc1ccc(C(F)(F)F)cn1] | 100 | 5/1 | 33 | | 40 | | 472.46 |
| 142 | 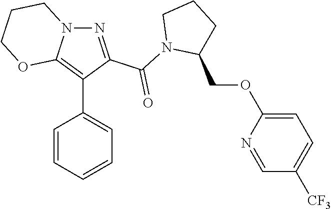 [SMILES: O=C(c1nn2c(c1-c1ccccc1)OCCC2)N1CCC[C@H]1COc1ccc(C(F)(F)F)cn1] | 100 | 6/1 | 42 | | | | 472.46 |
| 143 | 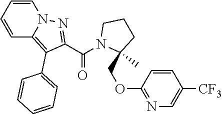 [SMILES: C[C@@]1(COc2ccc(C(F)(F)F)cn2)CCCN1C(=O)c1nn2ccccc2c1-c1ccccc1] | 98 | 210/45 | 31 | | 3 | | 480.48 |
| 144 | 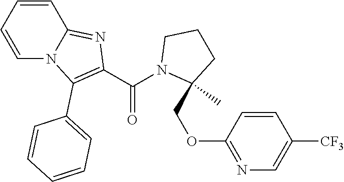 [SMILES: C[C@@]1(COc2ccc(C(F)(F)F)cn2)CCCN1C(=O)c1nc2ccccn2c1-c1ccccc1] | 98 | 250/53 | 13 | | 4 | | 480.48 |
| 145 | 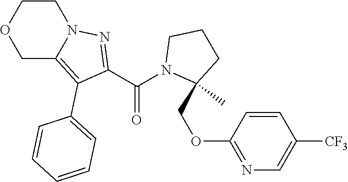 [SMILES: C[C@@]1(COc2ccc(C(F)(F)F)cn2)CCCN1C(=O)c1nn2c(c1-c1ccccc1)COCC2] | 96 | 270/56 | 5 | | 12 | | 486.59 |
| 146 | 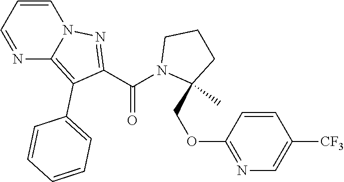 [SMILES: C[C@@]1(COc2ccc(C(F)(F)F)cn2)CCCN1C(=O)c1nn2cccnc2c1-c1ccccc1] | 99 | 350/74 | 27 | | 0 | | 481.47 |
| 147 | 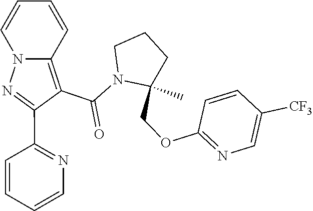 [SMILES: C[C@@]1(COc2ccc(C(F)(F)F)cn2)CCCN1C(=O)c1c(-c2ccccn2)nn2ccccc12] | 100 | 43/9 | 25 | | 2 | | 481.47 |
| 148 | 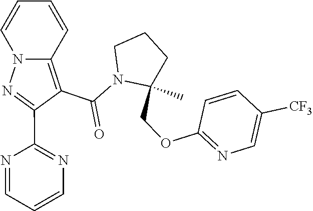 [SMILES: C[C@@]1(COc2ccc(C(F)(F)F)cn2)CCCN1C(=O)c1c(-c2ncccn2)nn2ccccc12] | 98 | 120/26 | 19 | | 8 | | 482.46 |
| 149 | 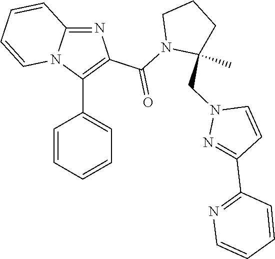 [SMILES: C[C@@]1(Cn2ccc(-c3ccccn3)n2)CCCN1C(=O)c1nc2ccccn2c1-c1ccccc1] | 89 | 570/120 | 13 | | 28 | | 462.55 |
| 150 | 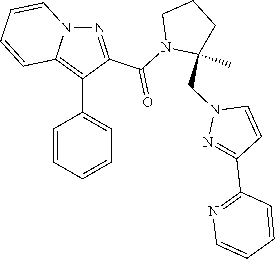 [SMILES: C[C@@]1(Cn2ccc(-c3ccccn3)n2)CCCN1C(=O)c1nn2ccccc2c1-c1ccccc1] | 97 | 340/72 | 11 | | 16 | | 462.55 |
| 151 | 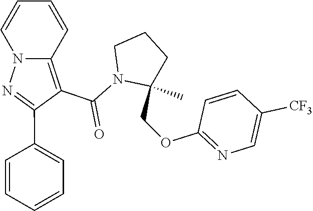 [SMILES: C[C@@]1(COc2ccc(C(F)(F)F)cn2)CCCN1C(=O)c1c(-c2ccccc2)nn2ccccc12] | 99 | 90/19 | 34 | | 7 | | 480.48 |
| 152 | 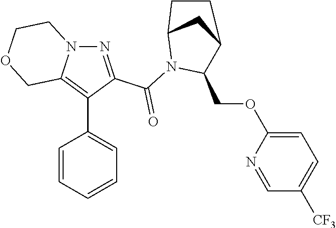 [SMILES: O=C(c1nn2c(c1-c1ccccc1)COCC2)N1[C@@H]2CC[C@@H](C2)[C@H]1COc1ccc(C(F)(F)F)cn1] | 58 | 2400/510 | 16 | | 3 | | 498.50 |
| 153 | 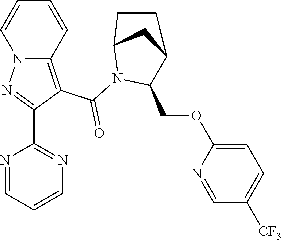 [SMILES: O=C(c1c(-c2ncccn2)nn2ccccc12)N1[C@@H]2CC[C@@H](C2)[C@H]1COc1ccc(C(F)(F)F)cn1] | 90 | 190/40 | 35 | | 0 | | 494.47 |
| 154 | 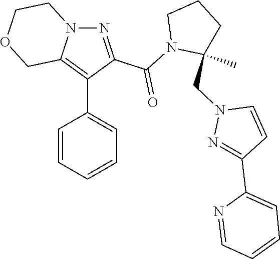 [SMILES: C[C@@]1(Cn2ccc(-c3ccccn3)n2)CCCN1C(=O)c1nn2c(c1-c1ccccc1)COCC2] | 83 | 520/110 | 5 | | 23 | | 468.55 |
| 155 | 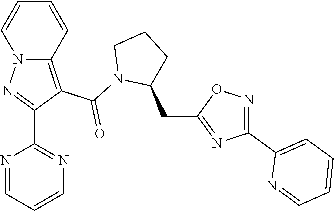 [SMILES: O=C(c1c(-c2ncccn2)nn2ccccc12)N1CCC[C@H]1Cc1nc(-c2ccccn2)no1] | 69 | 410/87 | 25 | | 12 | | 452.47 |
| 156 | 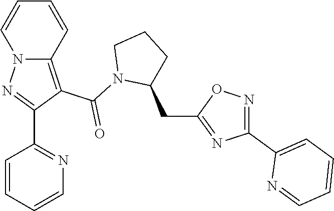 [SMILES: O=C(c1c(-c2ccccn2)nn2ccccc12)N1CCC[C@H]1Cc1nc(-c2ccccn2)no1] | 97 | 23/5 | 50 | | 6 | | 451.48 |
| 157 | 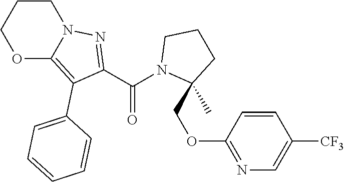 [SMILES: C[C@@]1(COc2ccc(C(F)(F)F)cn2)CCCN1C(=O)c1nn2c(c1-c1ccccc1)OCCC2] | 100 | 190/39 | 16 | | 7 | | 486.49 |
| 158 | 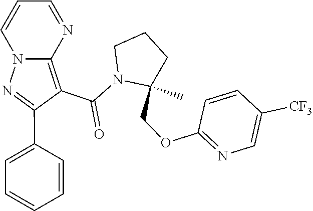 [SMILES: C[C@@]1(COc2ccc(C(F)(F)F)cn2)CCCN1C(=O)c1c(-c2ccccc2)nn2cccnc12] | 93 | 120/26 | 46 | | 2 | | 481.47 |
| 159 | 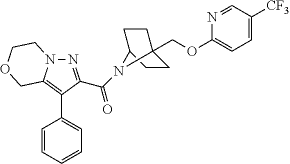 [SMILES: O=C(c1nn2c(c1-c1ccccc1)COCC2)N1C2CCC1(COc1ccc(C(F)(F)F)cn1)CC2] | 86 | 48/10 | 15 | | | | 498.50 |
| 160 | 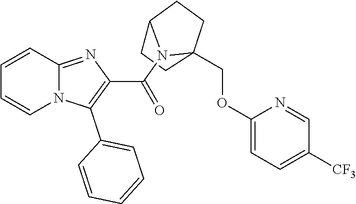 [SMILES: O=C(c1nc2ccccn2c1-c1ccccc1)N1C2CCC1(COc1ccc(C(F)(F)F)cn1)CC2] | 98 | 24/5 | 2 | | | | 492.49 |
| 161 | 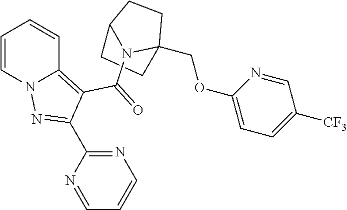 [SMILES: O=C(c1c(-c2ncccn2)nn2ccccc12)N1C2CCC1(COc1ccc(C(F)(F)F)cn1)CC2] | 44 | | 6 | | | | 494.47 |
| 162 | 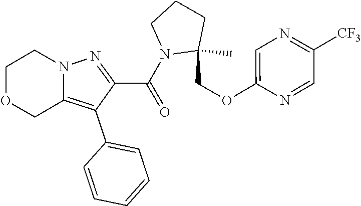 [SMILES: C[C@@]1(COc2cnc(C(F)(F)F)cn2)CCCN1C(=O)c1nn2c(c1-c1ccccc1)COCC2] | 36 | | 0 | | | | 487.47 |
| 163 | 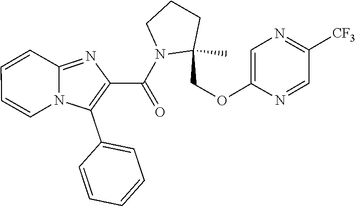 [SMILES: C[C@@]1(COc2cnc(C(F)(F)F)cn2)CCCN1C(=O)c1nc2ccccn2c1-c1ccccc1] | 75 | 771/162 | 11 | | | | 481.47 |
| 164 | 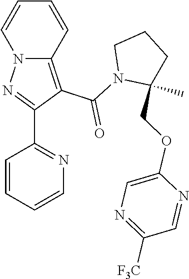 [SMILES: C[C@@]1(COc2cnc(C(F)(F)F)cn2)CCCN1C(=O)c1c(-c2ccccn2)nn2ccccc12] | 84 | 305/64 | 20 | | | | 482.46 |
| 165 | 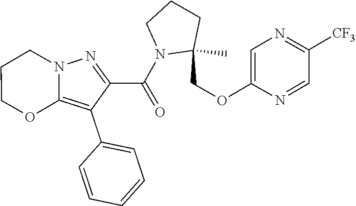 [SMILES: C[C@@]1(COc2cnc(C(F)(F)F)cn2)CCCN1C(=O)c1nn2c(c1-c1ccccc1)OCCC2] | 50 | | 6 | | | | 487.47 |
| 166 | 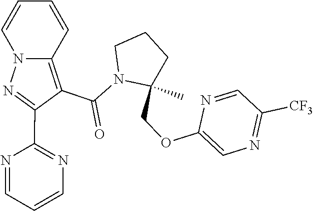 [SMILES: C[C@@]1(COc2cnc(C(F)(F)F)cn2)CCCN1C(=O)c1c(-c2ncccn2)nn2ccccc12] | 53 | | 10 | | | | 483.45 |
| 167 | 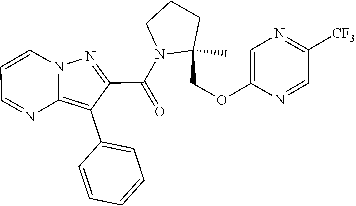 [SMILES: C[C@@]1(COc2cnc(C(F)(F)F)cn2)CCCN1C(=O)c1nn2cccnc2c1-c1ccccc1] | 82 | 326/69 | 20 | | | | 482.46 |
| 168 | 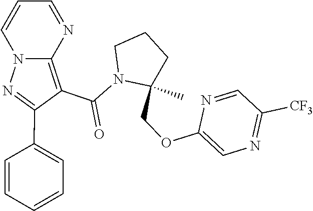 [SMILES: C[C@@]1(COc2cnc(C(F)(F)F)cn2)CCCN1C(=O)c1c(-c2ccccc2)nn2cccnc12] | 72 | 475/100 | 28 | | | | 482.46 |
| 169 | 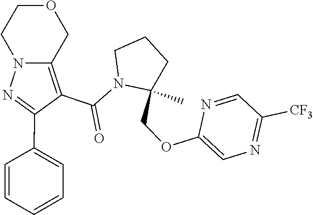 [SMILES: C[C@@]1(COc2cnc(C(F)(F)F)cn2)CCCN1C(=O)c1c(-c2ccccc2)nn2c1COCC2] | 2 | | 15 | | | | 487.47 |
| 170 | 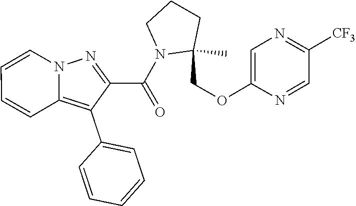 [SMILES: C[C@@]1(COc2cnc(C(F)(F)F)cn2)CCCN1C(=O)c1nn2ccccc2c1-c1ccccc1] | 85 | 460/97 | 23 | | | | 481.47 |
| 171 | 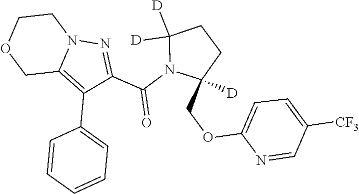 [SMILES: [2H]C1([2H])CC[C@@]([2H])(COc2ccc(C(F)(F)F)cn2)N1C(=O)c1nn2c(c1-c1ccccc1)COCC2] | 100 | 10/2 | 26 | | 0 | | 475.48 |
| 172 | 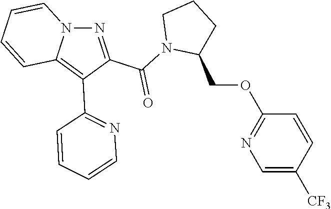 [SMILES: O=C(c1nn2ccccc2c1-c1ccccn1)N1CCC[C@H]1COc1ccc(C(F)(F)F)cn1] | 100 | 3/0.7 | 40 | | 4 | | 467.44 |
| 173 | 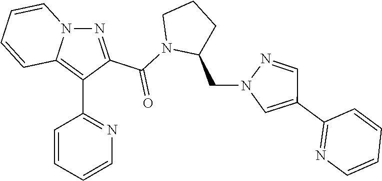 [SMILES: O=C(c1nn2ccccc2c1-c1ccccn1)N1CCC[C@H]1Cn1cc(-c2ccccn2)cn1] | 100 | 8/2 | 82 | 260/45 | 3 | | 449.51 |
| 174 | 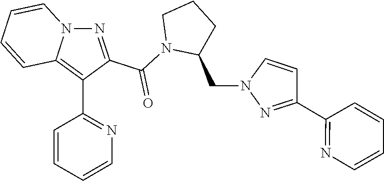 [SMILES: O=C(c1nn2ccccc2c1-c1ccccn1)N1CCC[C@H]1Cn1ccc(-c2ccccn2)n1] | 100 | 5/0.9 | 56 | | 0 | | 449.51 |
| 175 | 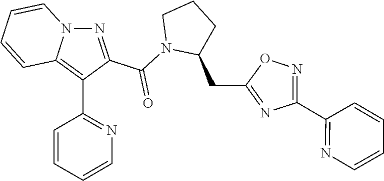 [SMILES: O=C(c1nn2ccccc2c1-c1ccccn1)N1CCC[C@H]1Cc1nc(-c2ccccn2)no1] | 93 | 11/2 | 29 | | | | 451.48 |
| 176 | 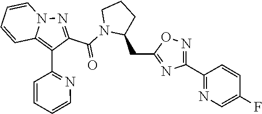 [SMILES: O=C(c1nn2ccccc2c1-c1ccccn1)N1CCC[C@H]1Cc1nc(-c2ccc(F)cn2)no1] | 93 | 12/2 | 40 | | | | 469.47 |
| 177 | 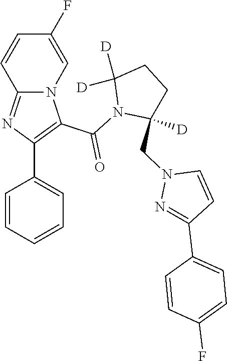 [SMILES: [2H]C1([2H])CC[C@@]([2H])(Cn2ccc(-c3ccc(F)cc3)n2)N1C(=O)c1c(-c2ccccc2)nc2ccc(F)cn12] | 95 | 20/4 | 85 | 371/64 | 91 | 90/49 | 486.53 |
| 178 | 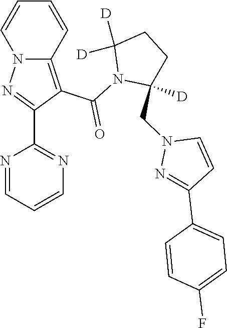 [SMILES: [2H]C1([2H])CC[C@@]([2H])(Cn2ccc(-c3ccc(F)cc3)n2)N1C(=O)c1c(-c2ncccn2)nn2ccccc12] | 92 | 4/0.8 | 95 | 15/3 | 53 | | 470.52 |
| 179 | 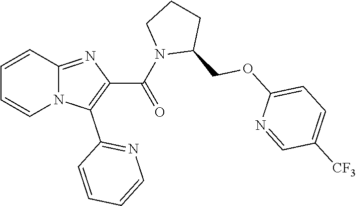 [SMILES: O=C(c1nc2ccccn2c1-c1ccccn1)N1CCC[C@H]1COc1ccc(C(F)(F)F)cn1] | 99 | 8/2 | 12 | | | | 467.44 |
| 180 | 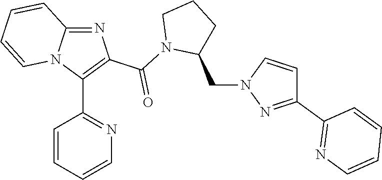 [SMILES: O=C(c1nc2ccccn2c1-c1ccccn1)N1CCC[C@H]1Cn1ccc(-c2ccccn2)n1] | 97 | 17/4 | 27 | | | | 449.51 |
| 181 | 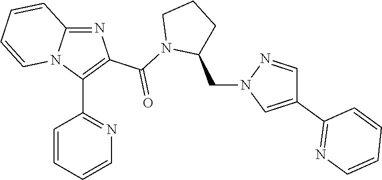 [SMILES: O=C(c1nc2ccccn2c1-c1ccccn1)N1CCC[C@H]1Cn1cc(-c2ccccn2)cn1] | 91 | 40/9 | 39 | | 0 | | 449.51 |
| 182 | 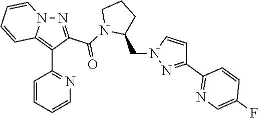 [SMILES: O=C(c1nn2ccccc2c1-c1ccccn1)N1CCC[C@H]1Cn1ccc(-c2ccc(F)cn2)n1] | 96 | 19/4 | 59 | | | | 467.50 |
| 183 | 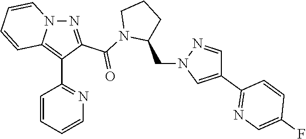 [SMILES: O=C(c1nn2ccccc2c1-c1ccccn1)N1CCC[C@H]1Cn1cc(-c2ccc(F)cn2)cn1] | 94 | 38/9 | 67 | | 0 | | 467.50 |
| 184 | 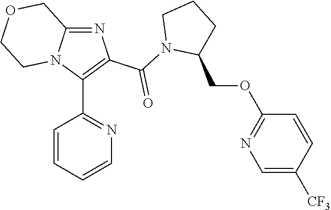 [SMILES: O=C(c1nc2n(c1-c1ccccn1)CCOC2)N1CCC[C@H]1COc1ccc(C(F)(F)F)cn1] | 42 | | 10 | | | | 473.45 |
| 185 | 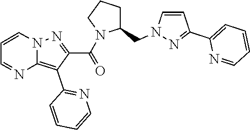 [SMILES: O=C(c1nn2cccnc2c1-c1ccccn1)N1CCC[C@H]1Cn1ccc(-c2ccccn2)n1] | 94 | 16/3 | 61 | | | | 450.50 |
| 186 | 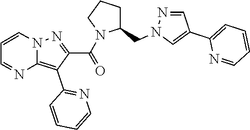 [SMILES: O=C(c1nn2cccnc2c1-c1ccccn1)N1CCC[C@H]1Cn1cc(-c2ccccn2)cn1] | 86 | 21/4 | 65 | | 3 | | 450.50 |
| 187 | 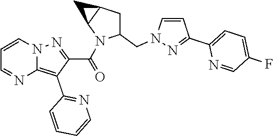 [SMILES: O=C(c1nn2cccnc2c1-c1ccccn1)N1C(Cn2ccc(-c3ccc(F)cn3)n2)C[C@@H]2C[C@@H]21] | 100 | 14/3 | 34 | | 0 | | 480.50 |
| 188 | 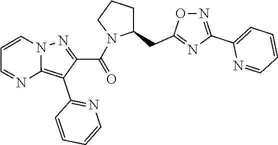 [SMILES: O=C(c1nn2cccnc2c1-c1ccccn1)N1CCC[C@H]1Cc1nc(-c2ccccn2)no1] | 82 | 29/6 | 23 | | | | 452.47 |
| 189 | 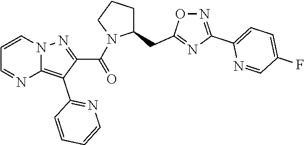 [SMILES: O=C(c1nn2cccnc2c1-c1ccccn1)N1CCC[C@H]1Cc1nc(-c2ccc(F)cn2)no1] | 94 | 18/4 | 43 | | | | 470.46 |
| 190 | 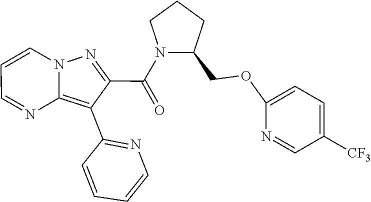 [SMILES: O=C(c1nn2cccnc2c1-c1ccccn1)N1CCC[C@H]1COc1ccc(C(F)(F)F)cn1] | 91 | 5/1 | 28 | | | | 468.43 |
| 191 | 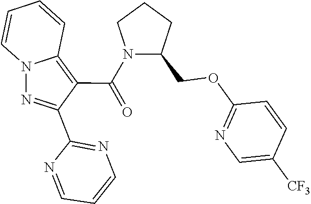 [SMILES: O=C(c1c(-c2ncccn2)nn2ccccc12)N1CCC[C@H]1COc1ccc(C(F)(F)F)cn1] | 100 | 49/10 | 11 | | | | 468.43 |
| 192 | 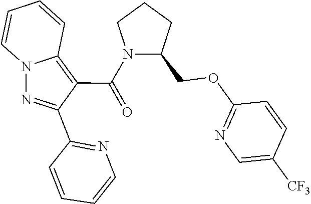 [SMILES: O=C(c1c(-c2ccccn2)nn2ccccc12)N1CCC[C@H]1COc1ccc(C(F)(F)F)cn1] | 88 | 11/2 | 49 | | | | 467.44 |
| 193 | 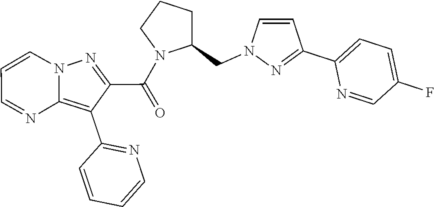 [SMILES: O=C(c1nn2cccnc2c1-c1ccccn1)N1CCC[C@H]1Cn1ccc(-c2ccc(F)cn2)n1] | 91 | 16/3 | 71 | | | | 468.49 |
| 194 | 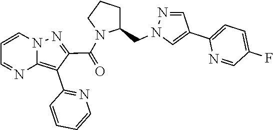 [SMILES: O=C(c1nn2cccnc2c1-c1ccccn1)N1CCC[C@H]1Cn1cc(-c2ccc(F)cn2)cn1] | 89 | 3/1 | 82 | 180/31 | 0 | | 468.49 |
| 195 | 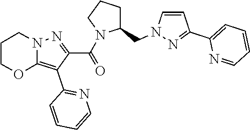 [SMILES: O=C(c1nn2c(c1-c1ccccn1)OCCC2)N1CCC[C@H]1Cn1ccc(-c2ccccn2)n1] | 100 | 15/3 | 29 | | | | 455.51 |
| 196 | 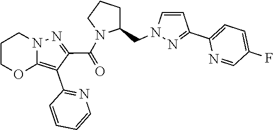 [SMILES: O=C(c1nn2c(c1-c1ccccn1)OCCC2)N1CCC[C@H]1Cn1ccc(-c2ccc(F)cn2)n1] | 99 | 24/5 | 39 | | | | 473.50 |
| 197 | 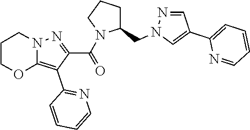 [SMILES: O=C(c1nn2c(c1-c1ccccn1)OCCC2)N1CCC[C@H]1Cn1cc(-c2ccccn2)cn1] | 97 | 31/7 | 39 | | 2 | | 455.51 |
| 198 | 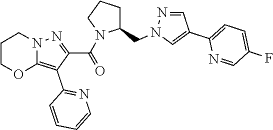 [SMILES: O=C(c1nn2c(c1-c1ccccn1)OCCC2)N1CCC[C@H]1Cn1cc(-c2ccc(F)cn2)cn1] | 84 | 140/29 | 50 | | 8 | | 473.50 |
| 199 | 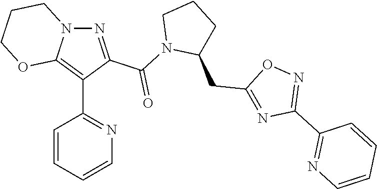 [SMILES: O=C(c1nn2c(c1-c1ccccn1)OCCC2)N1CCC[C@H]1Cc1nc(-c2ccccn2)no1] | 64 | | 8 | | | | 457.48 |
| 200 | 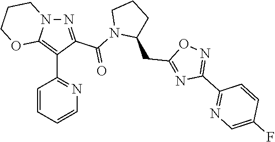 [SMILES: O=C(c1nn2c(c1-c1ccccn1)OCCC2)N1CCC[C@H]1Cc1nc(-c2ccc(F)cn2)no1] | 78 | 294/62 | 15 | | | | 457.48 |
| 201 | 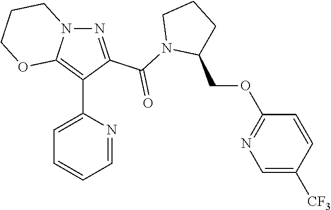 [SMILES: O=C(c1nn2c(c1-c1ccccn1)OCCC2)N1CCC[C@H]1COc1ccc(C(F)(F)F)cn1] | 100 | 9/2 | 19 | | | | 473.45 |
| 202 | 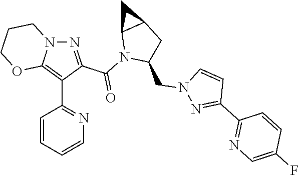 [SMILES: O=C(c1nn2c(c1-c1ccccn1)OCCC2)N1[C@H](Cn2ccc(-c3ccc(F)cn3)n2)C[C@@H]2C[C@@H]21] | 87 | 64/14 | 14 | | 3 | | 485.51 |
| 203 | 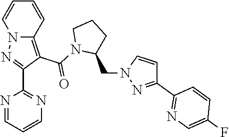 [SMILES: O=C(c1c(-c2ncccn2)nn2ccccc12)N1CCC[C@H]1Cn1ccc(-c2ccc(F)cn2)n1] | 98 | 17/4 | 78 | | | | 468.49 |
| 204 | 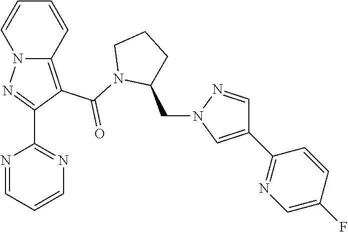 [SMILES: O=C(c1c(-c2ncccn2)nn2ccccc12)N1CCC[C@H]1Cn1cc(-c2ccc(F)cn2)cn1] | 53 | | 66 | | | | 468.49 |
| 205 | 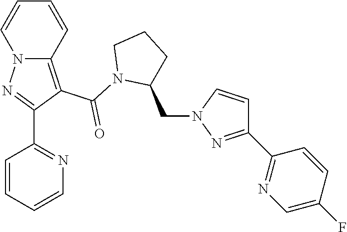 [SMILES: O=C(c1c(-c2ccccn2)nn2ccccc12)N1CCC[C@H]1Cn1ccc(-c2ccc(F)cn2)n1] | 99 | 12/3 | 98 | 24/4 | | | 467.50 |
| 206 | 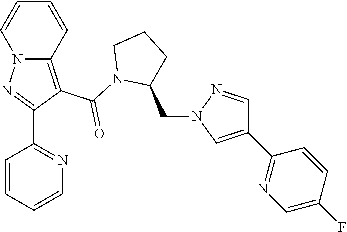 [SMILES: O=C(c1c(-c2ccccn2)nn2ccccc12)N1CCC[C@H]1Cn1cc(-c2ccc(F)cn2)cn1] | 67 | | 83 | 62/11 | | | 467.50 |
| 207 | 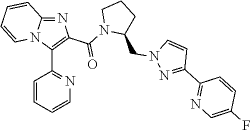 [SMILES: O=C(c1nc2ccccn2c1-c1ccccn1)N1CCC[C@H]1Cn1ccc(-c2ccc(F)cn2)n1] | 99 | 30/6 | 0 | | | | 467.50 |
| 208 | 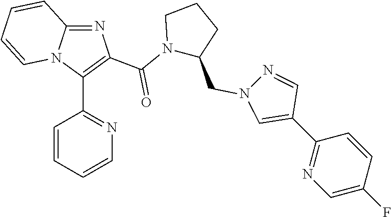 [SMILES: O=C(c1nc2ccccn2c1-c1ccccn1)N1CCC[C@H]1Cn1cc(-c2ccc(F)cn2)cn1] | 100 | 39/8 | 52 | | 0 | | 467.50 |
| 209 | 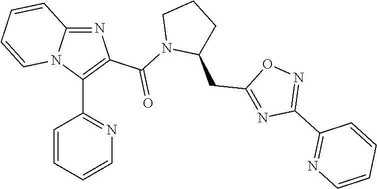 [SMILES: O=C(c1nc2ccccn2c1-c1ccccn1)N1CCC[C@H]1Cc1nc(-c2ccccn2)no1] | 75 | 300/63 | 4 | | | | 451.48 |
| 210 | 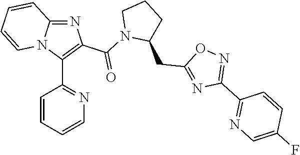 [SMILES: O=C(c1nc2ccccn2c1-c1ccccn1)N1CCC[C@H]1Cc1nc(-c2ccc(F)cn2)no1] | 85 | 190/40 | 12 | | | | 469.47 |
| 211 | 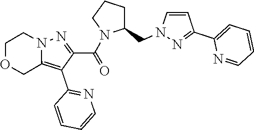 [SMILES: O=C(c1nn2c(c1-c1ccccn1)COCC2)N1CCC[C@H]1Cn1ccc(-c2ccccn2)n1] | 100 | 19/4 | 28 | | | | 455.51 |
| 212 | 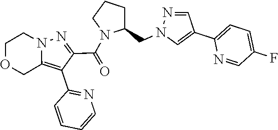 [SMILES: O=C(c1nn2c(c1-c1ccccn1)COCC2)N1CCC[C@H]1Cn1cc(-c2ccc(F)cn2)cn1] | 96 | 35/7 | 46 | | | | 473.50 |
| 213 | 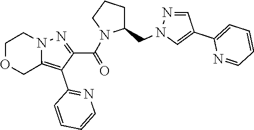 [SMILES: O=C(c1nn2c(c1-c1ccccn1)COCC2)N1CCC[C@H]1Cn1cc(-c2ccccn2)cn1] | 77 | 220/47 | 33 | | | | 455.51 |
| 214 | 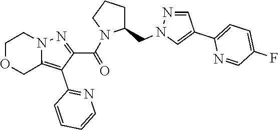 [SMILES: O=C(c1nn2c(c1-c1ccccn1)COCC2)N1CCC[C@H]1Cn1cc(-c2ccc(F)cn2)cn1] | 66 | | 32 | | | | 473.50 |
| 215 | 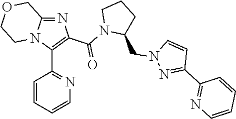 [SMILES: O=C(c1nc2n(c1-c1ccccn1)CCOC2)N1CCC[C@H]1Cn1ccc(-c2ccccn2)n1] | 64 | | 12 | | | | 455.51 |
| 216 | 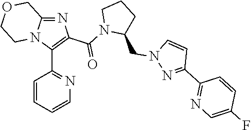 [SMILES: O=C(c1nc2n(c1-c1ccccn1)CCOC2)N1CCC[C@H]1Cn1ccc(-c2ccc(F)cn2)n1] | 70 | 160/34 | 24 | | | | 473.50 |
| 217 | 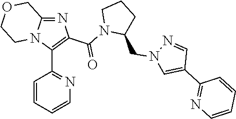 [SMILES: O=C(c1nc2n(c1-c1ccccn1)CCOC2)N1CCC[C@H]1Cn1cc(-c2ccccn2)cn1] | 76 | 210/45 | 57 | | | | 455.51 |
| 218 | 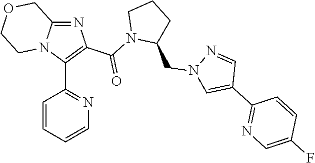 [SMILES: O=C(c1nc2n(c1-c1ccccn1)CCOC2)N1CCC[C@H]1Cn1cc(-c2ccc(F)cn2)cn1] | 79 | 120/25 | 74 | | | | 473.50 |
| 219 | 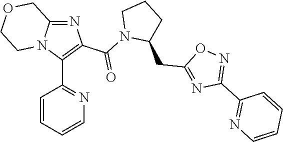 [SMILES: O=C(c1nc2n(c1-c1ccccn1)CCOC2)N1CCC[C@H]1Cc1nc(-c2ccccn2)no1] | 25 | | 0 | | | | 457.48 |
| 220 | 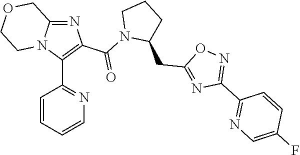 [SMILES: O=C(c1nc2n(c1-c1ccccn1)CCOC2)N1CCC[C@H]1Cc1nc(-c2ccc(F)cn2)no1] | 21 | | 0 | | | | 475.48 |
| 221 | 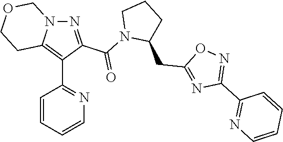 [SMILES: O=C(c1nn2c(c1-c1ccccn1)CCOC2)N1CCC[C@H]1Cc1nc(-c2ccccn2)no1] | 56 | | 0 | | | | 457.48 |
| 222 | 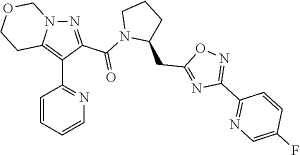 [SMILES: O=C(c1nn2c(c1-c1ccccn1)CCOC2)N1CCC[C@H]1Cc1nc(-c2ccc(F)cn2)no1] | 55 | | 12 | | | | 475.48 |
| 223 | 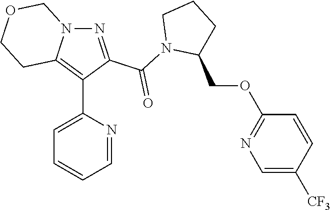 [SMILES: O=C(c1nn2c(c1-c1ccccn1)CCOC2)N1CCC[C@H]1COc1ccc(C(F)(F)F)cn1] | 97 | 5/1 | 21 | | | | 473.45 |
| 224 | 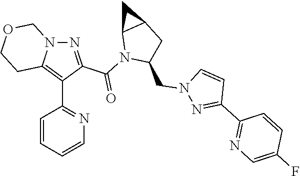 [SMILES: O=C(c1nn2c(c1-c1ccccn1)CCOC2)N1[C@H](Cn2ccc(-c3ccc(F)cn3)n2)C[C@@H]2C[C@@H]21] | 97 | 15/3 | 30 | | | | 485.51 |
| 225 | 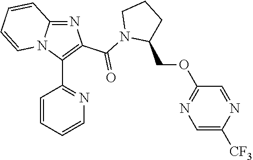 [SMILES: O=C(c1nc2ccccn2c1-c1ccccn1)N1CCC[C@H]1COc1cnc(C(F)(F)F)cn1] | | | | | | | 468.43 |
| 226 | 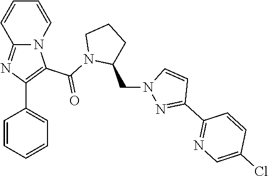 [SMILES: O=C(c1c(-c2ccccc2)nc2ccccn12)N1CCC[C@H]1Cn1ccc(-c2ccc(Cl)cn2)n1] | | | | | | | 482.96 |
| 227 | 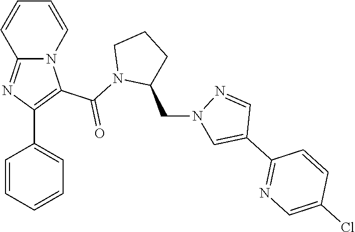 [SMILES: O=C(c1c(-c2ccccc2)nc2ccccn12)N1CCC[C@H]1Cn1cc(-c2ccc(Cl)cn2)cn1] | | | | | | | 482.96 |
| 228 | 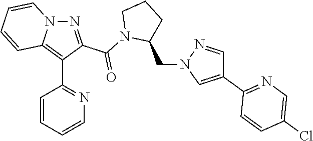 [SMILES: O=C(c1nn2ccccc2c1-c1ccccn1)N1CCC[C@H]1Cn1cc(-c2ccc(Cl)cn2)cn1] | | | | | | | 483.95 |
| 229 | 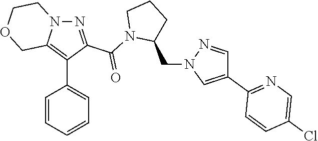 [SMILES: O=C(c1nn2c(c1-c1ccccc1)COCC2)N1CCC[C@H]1Cn1cc(-c2ccc(Cl)cn2)cn1] | | | | | | | 488.97 |
| 230 | 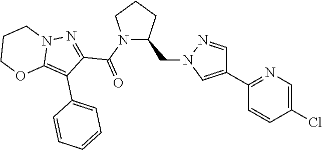 [SMILES: O=C(c1nn2c(c1-c1ccccc1)OCCC2)N1CCC[C@H]1Cn1cc(-c2ccc(Cl)cn2)cn1] | | | | | | | 488.97 |
| 231 | 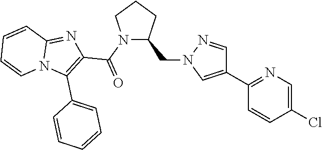 [SMILES: O=C(c1nc2ccccn2c1-c1ccccc1)N1CCC[C@H]1Cn1cc(-c2ccc(Cl)cn2)cn1] | | | | | | | 482.96 |
| 232 | 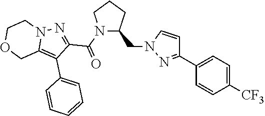 [SMILES: O=C(c1nn2c(c1-c1ccccc1)COCC2)N1CCC[C@H]1Cn1ccc(-c2ccc(C(F)(F)F)cc2)n1] | | | | | | | 521.53 |
| 233 | 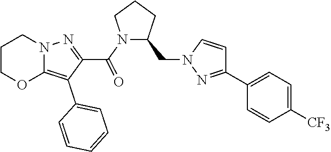 [SMILES: O=C(c1nn2c(c1-c1ccccc1)OCCC2)N1CCC[C@H]1Cn1ccc(-c2ccc(C(F)(F)F)cc2)n1] | | | | | | | 521.53 |
| 234 | 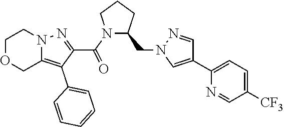 [SMILES: O=C(c1nn2c(c1-c1ccccc1)COCC2)N1CCC[C@H]1Cn1cc(-c2ccc(C(F)(F)F)cn2)cn1] | | | | | | | 522.52 |
| 235 | 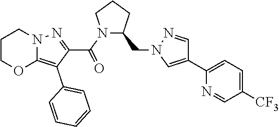 [SMILES: O=C(c1nn2c(c1-c1ccccc1)OCCC2)N1CCC[C@H]1Cn1cc(-c2ccc(C(F)(F)F)cn2)cn1] | | | | | | | 522.52 |
| 236 | 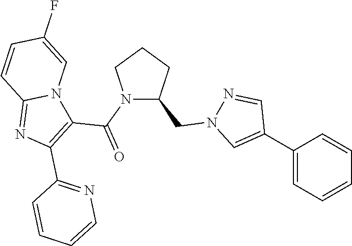 [SMILES: O=C(c1c(-c2ccccn2)nc2ccc(F)cn12)N1CCC[C@H]1Cn1cc(-c2ccccc2)cn1] | | | | | | | 466.51 |
| 237 | 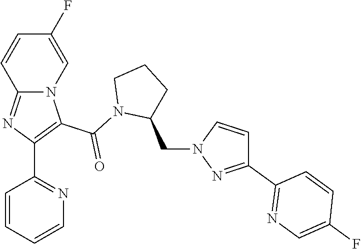 [SMILES: O=C(c1c(-c2ccccn2)nc2ccc(F)cn12)N1CCC[C@H]1Cn1ccc(-c2ccc(F)cn2)n1] | | | | | | | 485.49 |
| 238 | 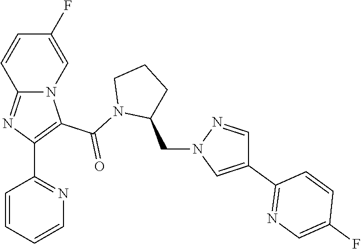 [SMILES: O=C(c1c(-c2ccccn2)nc2ccc(F)cn12)N1CCC[C@H]1Cn1cc(-c2ccc(F)cn2)cn1] | | | | | | | 485.49 |
| 239 | 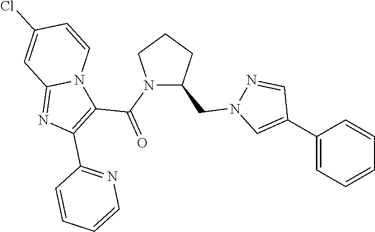 [SMILES: O=C(c1c(-c2ccccn2)nc2cc(Cl)ccn12)N1CCC[C@H]1Cn1cc(-c2ccccc2)cn1] | | | | | | | 482.96 |
| 240 | 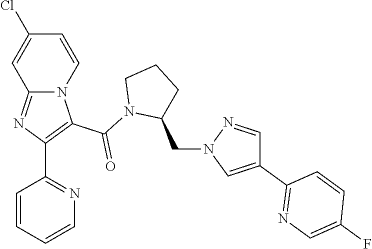 [SMILES: O=C(c1c(-c2ccccn2)nc2cc(Cl)ccn12)N1CCC[C@H]1Cn1cc(-c2ccc(F)cn2)cn1] | | | | | | | 501.94 |
| 241 | 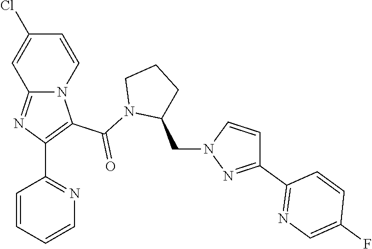 [SMILES: O=C(c1c(-c2ccccn2)nc2cc(Cl)ccn12)N1CCC[C@H]1Cn1ccc(-c2ccc(F)cn2)n1] | | | | | | | 501.94 |
| 242 | 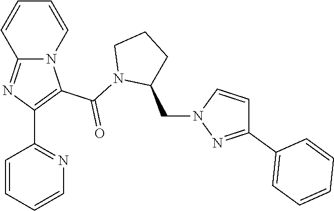 [SMILES: O=C(c1c(-c2ccccn2)nc2ccccn12)N1CCC[C@H]1Cn1ccc(-c2ccccc2)n1] | | | | | | | 448.52 |
| 243 | 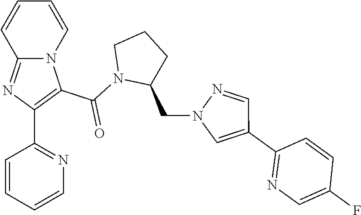 [SMILES: O=C(c1c(-c2ccccn2)nc2ccccn12)N1CCC[C@H]1Cn1cc(-c2ccc(F)cn2)cn1] | | | | | | | 467.50 |
| 244 | 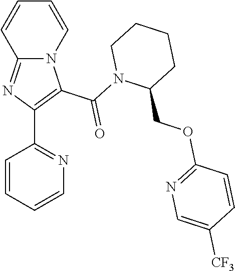 [SMILES: O=C(c1c(-c2ccccn2)nc2ccccn12)N1CCCC[C@H]1COc1ccc(C(F)(F)F)cn1] | | | | | | | 481.47 |
| 245 | 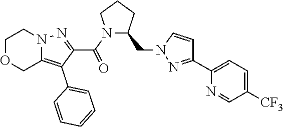 [SMILES: O=C(c1nn2c(c1-c1ccccc1)COCC2)N1CCC[C@H]1Cn1ccc(-c2ccc(C(F)(F)F)cn2)n1] | | | | | | | 522.52 |
| 246 | 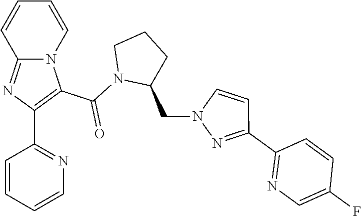 [SMILES: O=C(c1c(-c2ccccn2)nc2ccccn12)N1CCC[C@H]1Cn1ccc(-c2ccc(F)cn2)n1] | | | | | | | 467.50 |
| 247 | 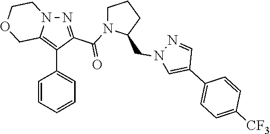 [SMILES: O=C(c1nn2c(c1-c1ccccc1)COCC2)N1CCC[C@H]1Cn1cc(-c2ccc(C(F)(F)F)cc2)cn1] | | | | | | | 521.53 |
| 248 | 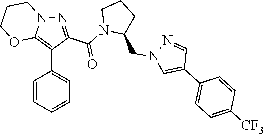 [SMILES: O=C(c1nn2c(c1-c1ccccc1)OCCC2)N1CCC[C@H]1Cn1cc(-c2ccc(C(F)(F)F)cc2)cn1] | | | | | | | 521.53 |
| 249 | 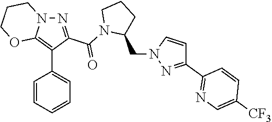 [SMILES: O=C(c1nn2c(c1-c1ccccc1)OCCC2)N1CCC[C@H]1Cn1ccc(-c2ccc(C(F)(F)F)cn2)n1] | | | | | | | 522.52 |
| 250 | 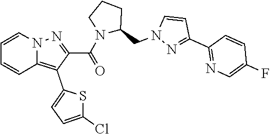 [SMILES: O=C(c1nn2ccccc2c1-c1ccc(Cl)s1)N1CCC[C@H]1Cn1ccc(-c2ccc(F)cn2)n1] | | | | | | | 506.98 |
| 251 | 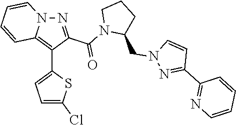 [SMILES: O=C(c1nn2ccccc2c1-c1ccc(Cl)s1)N1CCC[C@H]1Cn1ccc(-c2ccccn2)n1] | | | | | | | 488.99 |
| 252 | 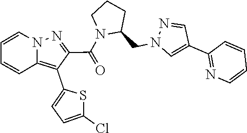 [SMILES: O=C(c1nn2ccccc2c1-c1ccc(Cl)s1)N1CCC[C@H]1Cn1cc(-c2ccccn2)cn1] | | | | | | | 488.99 |
| 253 | 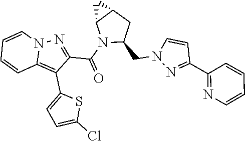 [SMILES: O=C(c1nn2ccccc2c1-c1ccc(Cl)s1)N1[C@H](Cn2ccc(-c3ccccn3)n2)C[C@H]2C[C@H]21] | | | | | | | 501.00 |
| 254 | 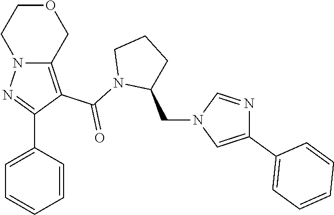 [SMILES: O=C(c1c(-c2ccccc2)nn2c1COCC2)N1CCC[C@H]1Cn1cnc(-c2ccccc2)c1] | | | | | | | 453.54 |
| 255 | 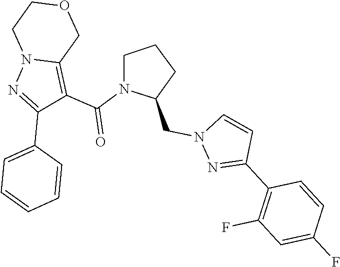 [SMILES: O=C(c1c(-c2ccccc2)nn2c1COCC2)N1CCC[C@H]1Cn1ccc(-c2ccc(F)cc2F)n1] | | | | | | | 489.52 |
| 256 | 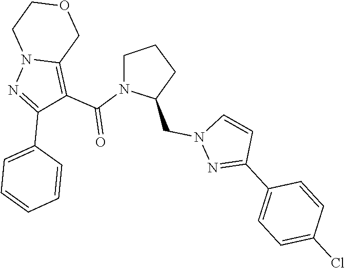 [SMILES: O=C(c1c(-c2ccccc2)nn2c1COCC2)N1CCC[C@H]1Cn1ccc(-c2ccc(Cl)cc2)n1] | | | | | | | 487.98 |
| 257 | 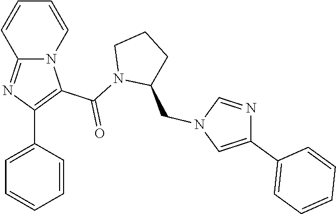 [SMILES: O=C(c1c(-c2ccccc2)nc2ccccn12)N1CCC[C@H]1Cn1cnc(-c2ccccc2)c1] | | | | | | | 447.53 |
| 258 | 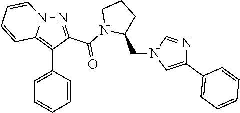 [SMILES: O=C(c1nn2ccccc2c1-c1ccccc1)N1CCC[C@H]1Cn1cnc(-c2ccccc2)c1] | | | | | | | 447.53 |
| 259 | 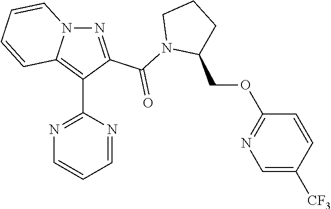 [SMILES: O=C(c1nn2ccccc2c1-c1ncccn1)N1CCC[C@H]1COc1ccc(C(F)(F)F)cn1] | | | | | | | 468.43 |
| 260 | 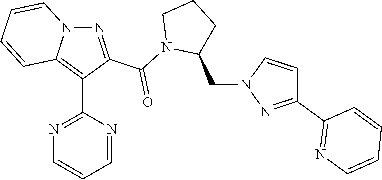 [SMILES: O=C(c1nn2ccccc2c1-c1ncccn1)N1CCC[C@H]1Cn1ccc(-c2ccccn2)n1] | | | | | | | 450.50 |
| 261 | 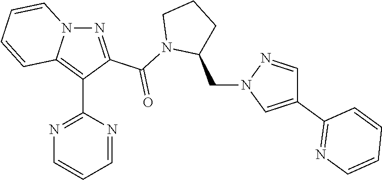 [SMILES: O=C(c1nn2ccccc2c1-c1ncccn1)N1CCC[C@H]1Cn1cc(-c2ccccn2)cn1] | | | | | | | 450.50 |
| 262 | 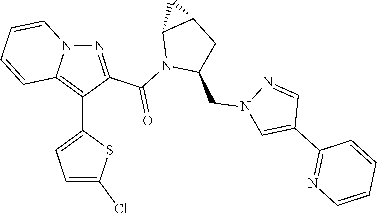 [SMILES: O=C(c1nn2ccccc2c1-c1ccc(Cl)s1)N1[C@H](Cn2cc(-c3ccccn3)cn2)C[C@H]2C[C@H]21] | | | | | | | 501.00 |
| 263 | 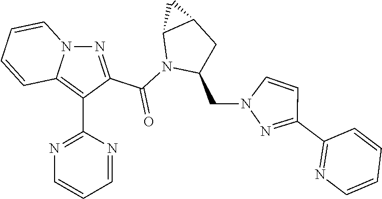 [SMILES: O=C(c1nn2ccccc2c1-c1ncccn1)N1[C@H](Cn2ccc(-c3ccccn3)n2)C[C@H]2C[C@H]21] | | | | | | | 462.51 |

[symbol]

While certain embodiments have been described in terms of the preferred embodiments, it is understood that variations and modifications will occur to those skilled in the art. Therefore, it is intended that the appended claims cover all such equivalent variations that come within the scope of the following claims.

What is claimed is:

1. A compound of Formula (I) or (II), 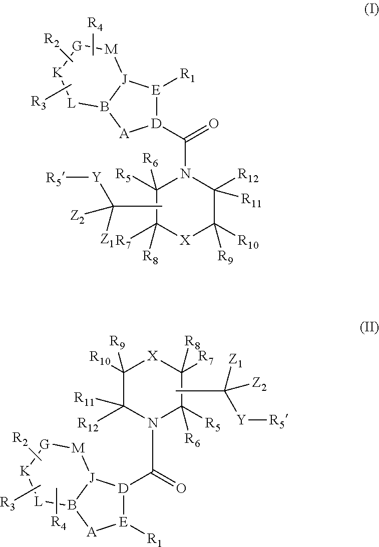
or a pharmaceutically acceptable salt, hydrate, solvate, polymorph, isomer, or combination thereof, wherein:
R₁ includes E as Carbon (C) but not Nitrogen (N), and in Formula I E is linked to J or D with a double bond or in Formula II E is linked to A or D with a double bond, and R₁ is selected from the group consisting of H, alkyl, alkoxy, cycloalkyl, phenyl, aromatic or aryl, heteroaryl that is optionally a 5- or 6 membered heteroaryl, substituted aromatic or aryl, and, substituted heteroaryl that is optionally a 5- or 6-membered heteroaryl; wherein if R₁ is heteroaryl, R₁ is optionally a 5- or 6-membered heteroaryl selected from the group consisting of pyrazolyl, triazolyl, oxazolyl, thiazolyl, thiophenyl, pyridinyl, pyrimidinyl, pyrazinyl, and pyridazinyl; wherein said aromatic, aryl or heteroaryl is unsubstituted, mono-substituted, or di-substituted, wherein the substituents are independently selected from the group consisting of (C₁₋₄)alkyl, (C₁₋₄)alkoxy, halogen, (C₁₋₃)fluoroalkyl, (C₁₋₃)fluoroalkoxy and (C₃₋₇)cycloalkyl;
R₂, R₃, and R₄ are independently selected from the group consisting of H, alkyl, substituted alkyl, (C₁₋₄)alkyl, (C₁₋₄)alkoxy, (C₁₋₃)fluoroalkyl, (C₁₋₃)fluoroalkoxy, (C₃₋₇)cycloalkyl, and halogen that is optionally F, Cl, or Br; wherein each of R₂, R₃ and R₄ is independently and optionally substituted at each substitutable position with up to three (3) substituents independently selected from one, two, or all R₂, R₃ and R₄;
R₅ is selected from the group consisting of H, F, CH₃, alkyl, substituted alkyl, (C₁₋₃)fluoroalkyl, cycloalkyl, and R₅ replaced with the carbon bearing Y, Z₁ and Z₂, wherein R₅′ is as defined herein;
R₅′ is selected from the group consisting of aromatic, aryl, heteroaryl, 5- or 6-membered heteroaryl, substituted aromatic or aryl, substituted heteroaryl or fused two heteroaryl ring systems, optionally comprising a 5- or 6-membered ring; wherein said aromatic, aryl or heteroaryl is unsubstituted, mono-, or di-substituted or tri-substituted, wherein the substituents are independently selected from the group consisting of (C₁₋₄)alkyl, (C₁₋₄)alkoxy, halogen, (C₁₋₃)fluoroalkyl, (C₁₋₃)fluoroalkoxy, (C₃₋₇)cycloalkyl, and (C₃₋₇)heterocycloalkyl;
R₆ is selected from the group consisting of H, F, CH₃, alkyl, substituted alkyl, (C₁₋₃)fluoroalkyl, cycloalkyl, and R₆ connected to either R₁₀ or R₁₁ as alkyl to form a (C₁₋₃)alkyl bridge cyclic structure;
R₇ is selected from the group consisting of H, F, CH₃, alkyl, substituted alkyl, (C₁₋₃)fluoroalkyl, cycloalkyl, and R₇ replaced with the carbon bearing Y, Z₁ and Z₂, wherein R₅′ is defined herein;
R₈ is selected from the group consisting of H, F, CH₃, alkyl, substituted alkyl, (C₁₋₃)fluoroalkyl, cycloalkyl, and Rₑ connected to either R₁₀, R₁₁ or R₁₂ as alkyl to form a (C₁₋₃)alkyl bridge cyclic structure;
R₉ is selected from the group consisting of H, F, CH₃, alkyl, substituted alkyl, (C₁₋₃)fluoroalkyl, cycloalkyl, and R₉ connected to either R₆ or R₁₂ as alkyl to form a (C₁₋₃)alkyl bridge cyclic structure;
R₁₀ is selected from the group consisting of H, F, CH₃, alkyl, substituted alkyl, (C₁₋₃)fluoroalkyl, cycloalkyl, and R₁₀ connected to R₆ or R₁₁ as alkyl to form a (C₁₋₃)alkyl bridge cyclic structure;
R₁₁ is selected from the group consisting of H, F, CH₃, alkyl, substituted alkyl, (C₁₋₃)fluoroalkyl, cycloalkyl, and R₁₁ connected to R₆ as alkyl to form a (C₁₋₃)alkyl bridge cyclic structure;
R₁₂ is selected from the group consisting of H, F, CH₃, alkyl, substituted alkyl, (C₁₋₃)fluoroalkyl, cycloalkyl, and R₁₂ connected to R₉ as alkyl to form a (C₁₋₃)alkyl bridge cyclic structure;
X is selected from the group consisting of absent to optionally provide five membered pyrrolidine ring, CH₂, O, and CRₐR_{b} where Rₐ and R_{b} are selected from the group consisting of alkyl, cycloalkyl, and Fluoroalkyl; and wherein:
the carbon atom at position 2 of the piperidine or pyrrolidine is optionally in absolute (S)-configuration; or,
the carbon atom at position 2 of the of the morpholine ring wherein X is oxygen and optionally in absolute (R)-configuration;
Y is selected from the group consisting of absent to provide R₅′ attached directly to the carbon bearing Z₁ and Z₂ groups; O; NH; CH₂OR₅′; CH₂; NRₐ wherein Rₐ is selected from the group consisting of alkyl, cycloalkyl, and heteroalkyl; and a 5 or 6-membered heteroaryl selected from the group consisting of pyrrolyl, pyrazolyl, triazolyl, oxazolyl, thiazolyl, and oxadiazolyl; and,
Z₁ and Z₂ are independently selected from the group consisting of H, F, (C₁₋₄)alkyl, (C₁₋₃)fluoroalkyl, (C₁₋₃)fluoroalkoxy, and (C₂₋₇)cycloalkyl;
and wherein:
fused or unfused ring system A-B-J-D-E is a five-membered heteroaryl optionally imidazole wherein A and J are Nitrogen while B, E, D are Carbon; pyrazole wherein A and B are Nitrogen while D, E, and J are Carbon; optionally fused or unfused with one or more additional ring systems;
the fused ring system B-J-M-G-K-L is an arrangements of these outlined variables to provide groups consisting of a 6-membered aromatic, 6-membered aryl, 6-membered substituted aromatic, 6-membered substituted aryl, 6-membered substituted heteroaryl, 6-membered unsubstituted heteroaryl, 5 or 6-membered cycloalkyl, and 5 or 6-membered heterocycloalkyl;
and wherein, optionally:
A is Nitrogen, optionally an Imidazole or Pyrazole;
B is Carbon or Nitrogen;
J is Carbon or Nitrogen;
D is Carbon;
E is Carbon, wherein R₁ is as defined above;
M is selected from the group consisting of Carbon, CH, CHR₂, CHR₃, CR₂R₃, CR₂, CR₃, CR₄, O, and N;
G is selected from the group consisting of Carbon, CH, CHR₂, CHR₃, CR₂R₃, CR₂, CR₃, CR₄, and O;
K is selected from the group consisting of Carbon, CH, CHR₂, CHR₃, CR₂R₃, CR₂, CR₃, CR₄, and O; and,
L is selected from the group consisting of Carbon, CH, CHR₂, CHR₃, CR₂R₃, CR₂, CR₃, CR₄, O, and N;
or a pharmaceutically acceptable salt, hydrate, solvate, polymorph, isomer, or combination thereof.

2. A compound of claim 1 having Formula I-a or II-a: 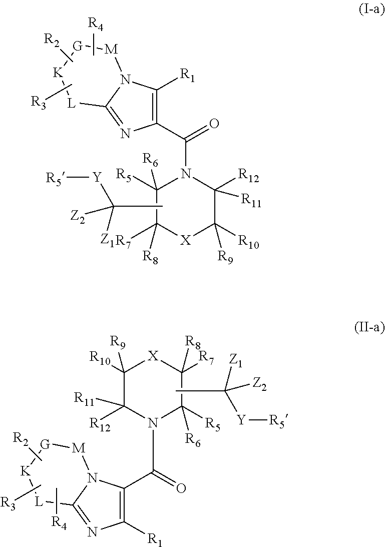
wherein:
R₁ is selected from the group consisting of H, alkyl, alkoxy, cycloalkyl, phenyl, aromatic or aryl, heteroaryl that is optionally a 5- or 6 membered heteroaryl, substituted aromatic or aryl, and, substituted heteroaryl that is optionally a 5- or 6-membered heteroaryl; wherein if R₁ is heteroaryl, R₁ is optionally a 5- or 6-membered heteroaryl selected from the group consisting of pyrazolyl, triazolyl, oxazolyl, thiazolyl, thiophenyl, pyridinyl, pyrimidinyl, pyrazinyl, and pyridazinyl; wherein said aromatic, aryl or heteroaryl is unsubstituted, mono-substituted, or di-substituted, wherein the substituents are independently selected from the group consisting of (C₁₋₄)alkyl, (C₁₋₄)alkoxy, halogen, (C₁₋₃)fluoroalkyl, (C₁₋₃)fluoroalkoxy and (C₃₋₇)cycloalkyl;
R₂, R₃, and R₄ are independently selected from the group consisting of H, alkyl, substituted alkyl, (C₁₋₄)alkyl, (C₁₋₄)alkoxy, (C₁₋₃)fluoroalkyl, (C₁₋₃)fluoroalkoxy, (C₃₋₇)cycloalkyl, halogen that is optionally F, Cl, or Br; wherein each of R₂, R₃ and R₄ is independently and optionally substituted at each substitutable position with up to three (3) substituents independently selected from one, two, or all R₂, R₃ and R₄;
R₅ is selected from the group consisting of H, F, CH₃, alkyl, substituted alkyl, (C₁₋₃)fluoroalkyl, cycloalkyl, and R₅ replaced with the carbon bearing Y, Z₁ and Z₂, wherein R₅′ is as defined herein;
R₅′ is selected from the group consisting of aromatic, aryl, heteroaryl, 5- or 6-membered heteroaryl, substituted aromatic or aryl, substituted heteroaryl or fused two heteroaryl ring systems, optionally comprising a 5- or 6-membered ring; wherein said aromatic, aryl or heteroaryl is unsubstituted, mono-, or di-substituted or tri-substituted, wherein the substituents are independently selected from the group consisting of (C₁₋₄)alkyl, (C₁₋₄)alkoxy, halogen, (C₁₋₃)fluoroalkyl, (C₁₋₃)fluoroalkoxy, (C₃₋₇)cycloalkyl, and (C₃₋₇)heterocycloalkyl;
R₆ is selected from the group consisting of H, F, CH₃, alkyl, substituted alkyl, (C₁₋₃)fluoroalkyl, cycloalkyl, and R₆ connected to either R₁₀ or R₁₁ as alkyl to form a (C₁₋₃)alkyl bridge cyclic structure;
R₇ is selected from the group consisting of H, F, CH₃, alkyl, substituted alkyl, (C₁₋₃)fluoroalkyl, cycloalkyl, and R₇ replaced with the carbon bearing Y, Z₁ and Z₂, wherein R₅′ is defined herein;
R₈ is selected from the group consisting of H, F, CH₃, alkyl, substituted alkyl, (C₁₋₃)fluoroalkyl, cycloalkyl, and R₈ connected to either R₁₁ or R₁₂ as alkyl to form a (C₁₋₃)alkyl bridge cyclic structure;
R₉ is selected from the group consisting of H, F, CH₃, alkyl, substituted alkyl, (C₁₋₃)fluoroalkyl, cycloalkyl, and R₉ connected to either R₆ or R₁₂ as alkyl to form a (C₁₋₃)alkyl bridge cyclic structure;
R₁₀ is selected from the group consisting of H, F, CH₃, alkyl, substituted alkyl, (C₁₋₃)fluoroalkyl, cycloalkyl, and R₁₀ connected to R₆ or R₁₁ as alkyl to form a (C₁₋₃)alkyl bridge cyclic structure;
R₁₁ is selected from the group consisting of H, F, CH₃, alkyl, substituted alkyl, (C₁₋₃)fluoroalkyl, cycloalkyl, and R₁₁ connected to R₆ as alkyl to form a (C₁₋₃)alkyl bridge cyclic structure;
R₁₂ is selected from the group consisting of H, F, CH₃, alkyl, substituted alkyl, (C₁₋₃)fluoroalkyl, cycloalkyl, and R₁₂ connected to R₉ as alkyl to form a (C₁₋₃)alkyl bridge cyclic structure;
X is selected from the group consisting of absent to optionally provide five membered pyrrolidine ring, CH₂, O, and CRₐR_{b} where Rₐ and R_{b} are selected from the group consisting of alkyl, cycloalkyl, and Fluoroalkyl; and wherein:
the carbon atom at position 2 of the piperidine or pyrrolidine ring is optionally in absolute (S)-configuration; or,
the carbon atom at position 2 of the of the morpholine ring wherein X is oxygen is optionally in absolute (R)-configuration;
Y is selected from the group consisting of absent to provide R₅′ attached directly to the carbon bearing Z₁ and Z₂ groups; O; NH; CH₂OR₅′; CH₂; NRₐ wherein Rₐ is selected from the group consisting of alkyl, cycloalkyl, and heteroalkyl; and a 5- or 6-membered heteroaryl selected from the group consisting of pyrrolyl, pyrazolyl, triazolyl, oxazolyl, thiazolyl, and oxadiazolyl;
Z₁ and Z₂ are independently selected from the group consisting of H, F, (C₁₋₄)alkyl, (C₁₋₃)fluoroalkyl, (C₁₋₃)fluoroalkoxy, and (C₂₋₇)cycloalkyl;
wherein:
the fused ring system B-J-M-G-K-L is selected from arrangements of these outlined variables to provide groups consisting of a 6-membered aromatic, 6-membered aryl, 6-membered substituted aromatic, 6-membered substituted aryl, 6-membered substituted heteroaryl, 6-membered unsubstituted heteroaryl, 5 or 6-membered cycloalkyl, and 5 or 6-membered heterocycloalkyl;
and preferably:
M is selected from the group consisting of Carbon, CH, CHR₂, CHR₃, CR₂R₃, CR₂, CR₃, CR₄, O, and N;
G is selected from the group consisting of Carbon, CH, CHR₂, CHR₃, CR₂R₃, CR₂, CR₃, CR₄, and O;
K is selected from the group consisting of Carbon, CH, CHR₂, CHR₃, CR₂R₃, CR₂, CR₃, CR₄, and O, or absent to provide 5-membered cycloalkyl, and heterocycloalkyl; and,
L is selected from the group consisting of Carbon, CH, CHR₂, CHR₃, CR₂R₃, CR₂, CR₃, CR₄, O, and, N;
or a pharmaceutically acceptable salt, hydrate, solvate, polymorph, isomer, or combination thereof.

3. A compound of claim 1 having Formula I-b or II-b: 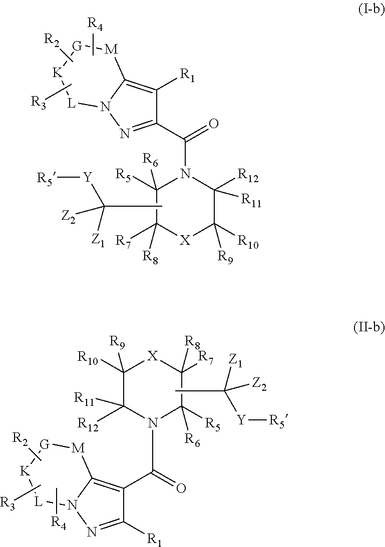
wherein:
R₁ is selected from the group consisting of H, alkyl, alkoxy, cycloalkyl, phenyl, aromatic or aryl, heteroaryl that is optionally a 5- or 6 membered heteroaryl, substituted aromatic or aryl, and, substituted heteroaryl that is optionally a 5- or 6-membered heteroaryl; wherein if R₁ is heteroaryl, R₁ is optionally a 5- or 6-membered heteroaryl selected from the group consisting of pyrazolyl, triazolyl, oxazolyl, thiazolyl, thiophenyl, pyridinyl, pyrimidinyl, pyrazinyl, and pyridazinyl; wherein said aromatic, aryl or heteroaryl is unsubstituted, mono-substituted, or di-substituted, wherein the substituents are independently selected from the group consisting of (C₁₋₄)alkyl, (C₁₋₄)alkoxy, halogen, (C₁₋₃)fluoroalkyl, (C₁₋₃)fluoroalkoxy and (C₃₋₇)cycloalkyl;
R₂, R₃, and R₄ are independently selected from the group consisting of H, halogen (such as F, Cl, Br), alkyl, substituted alkyl, (C₁₋₄)alkyl, (C₁₋₄)alkoxy, (C₁₋₃)fluoroalkyl, (C₁₋₃)fluoroalkoxy and (C₃₋₇)cycloalkyl; wherein each of R₂, R₃ and R₄ is independently and optionally substituted at each substitutable position with up to three (3) substituents independently selected from one, two, or all R₂, R₃ and R₄;
R₅ is selected from the group consisting of H, F, CH₃, alkyl, substituted alkyl, (C₁₋₃)fluoroalkyl, cycloalkyl, and R₅ replaced with the carbon bearing Y, Z₁ and Z₂, wherein R₅′ is as defined herein;
R₅′ is selected from the group consisting of aromatic, aryl, heteroaryl, 5- or 6-membered heteroaryl, substituted aromatic or aryl, substituted heteroaryl or fused two heteroaryl ring systems, optionally comprising a 5- or 6-membered ring; wherein said aromatic, aryl or heteroaryl is unsubstituted, mono-, or di-substituted or tri-substituted, wherein the substituents are independently selected from the group consisting of (C₁₋₄)alkyl, (C₁₋₄)alkoxy, halogen, (C₁₋₃)fluoroalkyl, (C₁₋₃)fluoroalkoxy, (C₃₋₇)cycloalkyl, and (C₃₋₇)heterocycloalkyl;
R₆ is selected from the group consisting of H, F, CH₃, alkyl, substituted alkyl, (C₁₋₃)fluoroalkyl, cycloalkyl, and R₆ connected to either R₁₀ or R₁₁ as alkyl to form a (C₁₋₃)alkyl bridge cyclic structure;
R₇ is selected from the group consisting of H, F, CH₃, alkyl, substituted alkyl, (C₁₋₃)fluoroalkyl, cycloalkyl, and R₇ replaced with the carbon bearing Y, Z₁ and Z₂, wherein R₅′ is defined herein;
R₈ is selected from the group consisting of H, F, CH₃, alkyl, substituted alkyl, (C₁₋₃)fluoroalkyl, cycloalkyl, and R₈ connected to either R₁₀, R₁₁ or R₁₂ as alkyl to form a (C₁₋₃)alkyl bridge cyclic structure;
R₉ is selected from the group consisting of H, F, CH₃, alkyl, substituted alkyl, (C₁₋₃)fluoroalkyl, cycloalkyl, and R₉ connected to either R₆ or R₁₂ as alkyl to form a (C₁₋₃)alkyl bridge cyclic structure;
R₁₀ is selected from the group consisting of H, F, CH₃, alkyl, substituted alkyl, (C₁₋₃)fluoroalkyl, cycloalkyl, and R₁₀connected to R₆ or R₁₁ as alkyl to form a (C₁₋₃)alkyl bridge cyclic structure;
R₁₁ is selected from the group consisting of H, F, CH₃, alkyl, substituted alkyl, (C₁₋₃)fluoroalkyl, cycloalkyl, and R₁₁ connected to R₆ as alkyl to form a (C₁₋₃)alkyl bridge cyclic structure;
R₁₂ is selected from the group consisting of H, F, CH₃, alkyl, substituted alkyl, (C₁₋₃)fluoroalkyl, cycloalkyl, and R₁₂ connected to R₉ as alkyl to form a (C₁₋₃)alkyl bridge cyclic structure;
X is selected from the group consisting of absent to optionally provide five membered pyrrolidine ring, CH₂, O, and CRₐR_{b} where Rₐ and R_{b} are selected from the group consisting of alkyl, cycloalkyl, and Fluoroalkyl; and wherein:
the carbon atom at position 2 of the piperidine or pyrrolidine is optionally in absolute (S)-configuration; or,
the carbon atom at position 2 of the of the morpholine ring wherein X is oxygen and optionally in absolute (R)-configuration;
Y is selected from the group consisting of absent to provide R₅′ attached directly to the carbon bearing Z₁ and Z₂ groups; O; NH; CH₂OR₅′; CH₂; NRₐ wherein Rₐ is selected from the group consisting of alkyl, cycloalkyl, and heteroalkyl; and a 5 or 6-membered heteroaryl selected from the group consisting of pyrrolyl, pyrazolyl, triazolyl, oxazolyl, thiazolyl, and oxadiazolyl;
Z₁ and Z₂ are independently selected from the group consisting of H, F, (C₁₋₄)alkyl, (C₁₋₃)fluoroalkyl, (C₁₋₃)fluoroalkoxy, and (C₂₋₇)cycloalkyl;
wherein:
the fused ring system B-J-M-G-K-L is selected from arrangements of these outlined variables to provide groups consisting of a 6-membered aromatic, 6-membered aryl, 6-membered substituted aromatic, 6-membered substituted aryl, 6-membered substituted heteroaryl, 6-membered unsubstituted heteroaryl, 5 or 6-membered cycloalkyl, and 5 or 6-membered heterocycloalkyl;
and preferably:
M is selected from the group consisting of Carbon, CH, CHR₂, CHR₃, CR₂R₃, CR₂, CR₃, CR₄, O, and N;
G is selected from the group consisting of Carbon, CH, CHR₂, CHR₃, CR₂R₃, CR₂, CR₃, CR₄, and O;
K is selected from the group consisting of Carbon, CH, CHR₂, CHR₃, CR₂R₃, CR₂, CR₃, CR₄, and O, or absent to provide 5-membered cycloalkyl, and heterocycloalkyl; and,
L is selected from the group consisting of Carbon, CH, CHR₂, CHR₃, CR₂R₃, CR₂, CR₃, CR₄, O, and N;
or a pharmaceutically acceptable salt, hydrate, solvate, polymorph, isomer, or combination thereof

4. A compound of claim 1 having Formula I-a4, I-a5, I-a6, II-a4, II-a5, or II-a6: 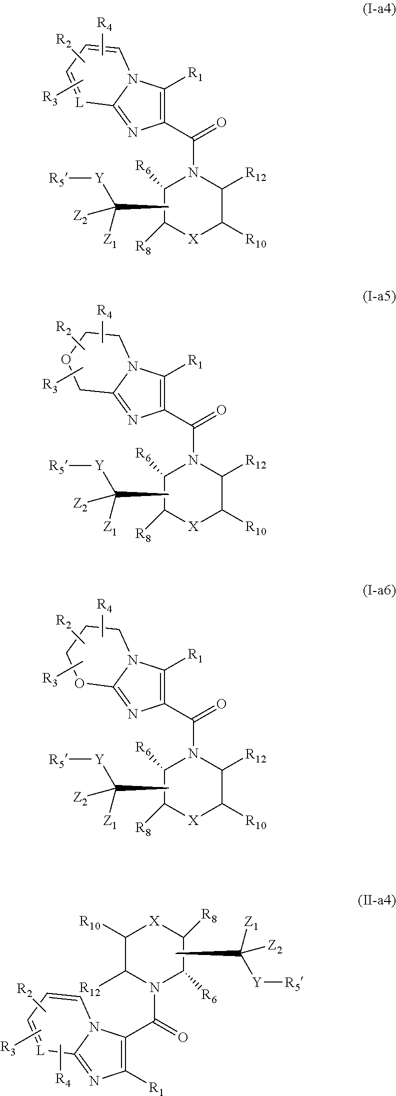 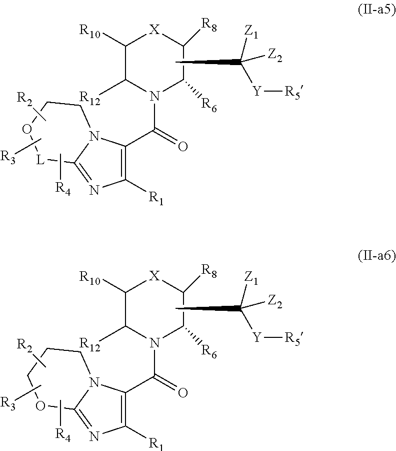
wherein:
R₁ is selected from the group consisting of H, alkyl, alkoxy, cycloalkyl, phenyl, aromatic or aryl, heteroaryl that is optionally a 5- or 6 membered heteroaryl, substituted aromatic or aryl, and, substituted heteroaryl that is optionally a 5- or 6-membered heteroaryl;
wherein if R₁ is heteroaryl, R₁ is optionally a 5- or 6-membered heteroaryl selected from the group consisting of pyrazolyl, triazolyl, oxazolyl, thiazolyl, thiophenyl, pyridinyl, pyrimidinyl, pyrazinyl, and pyridazinyl; wherein said aromatic, aryl or heteroaryl is unsubstituted, mono-substituted, or di-substituted, wherein the substituents are independently selected from the group consisting of (C₁₋₄)alkyl, (C₁₋₄)alkoxy, halogen, (C₁₋₃)fluoroalkyl, (C₁₋₃)fluoroalkoxy and (C₃₋₇)cycloalkyl;
R₂, R₃, and R₄ are independently selected from the group consisting of H, halogen (such as F, Cl, Br), alkyl, substituted alkyl, (C₁₋₄)alkyl, (C₁₋₄)alkoxy, (C₁₋₃)fluoroalkyl, (C₁₋₃)fluoroalkoxy and (C₃₋₇)cycloalkyl; wherein each of R₂, R₃ and R₄ is independently and optionally substituted at each substitutable position with up to three (3) substituents independently selected from one, two, or all R₂, R₃ and R₄;
R₅ is H or replaced with the carbon bearing Y, Z₁ and Z₂, wherein R₅′ is as defined herein;
R₅′ is selected from the group consisting of aromatic, aryl, heteroaryl, 5- or 6-membered heteroaryl, substituted aromatic or aryl, substituted heteroaryl or fused two heteroaryl ring systems, optionally comprising a 5- or 6-membered ring; wherein said aromatic, aryl or heteroaryl is unsubstituted, mono-, or di-substituted or tri-substituted, wherein the substituents are independently selected from the group consisting of (C₁₋₄)alkyl, (C₁₋₄)alkoxy, halogen, (C₁₋₃)fluoroalkyl, (C₁₋₃)fluoroalkoxy, (C₃₋₇)cycloalkyl, and (C₃₋₇)heterocycloalkyl;
R₆ is selected from the group consisting of H, F, CH₃, alkyl, substituted alkyl, (C₁₋₃)fluoroalkyl, cycloalkyl, and R₆ connected to either R₁₀ or R₁₁ as alkyl to form a (C₁₋₃)alkyl bridge cyclic structure;
R₇ is H or replaced with the carbon bearing Y, Z₁ and Z₂, wherein R₅′ is defined herein;
R₈ is selected from the group consisting of H, F, CH₃, alkyl, substituted alkyl, (C₁₋₃)fluoroalkyl, cycloalkyl, and R₈ connected to either R₁₀, R₁₁ or R₁₂ as alkyl to form a (C₁₋₃)alkyl bridge cyclic structure;
R₉ is selected from the group consisting of H, F, CH₃, alkyl, substituted alkyl, (C₁₋₃)fluoroalkyl, cycloalkyl, and R₉ connected to either R₆ or R₁₂ as alkyl to form a (C₁₋₃)alkyl bridge cyclic structure;
R₁₀ is selected from the group consisting of H, F, CH₃, alkyl, substituted alkyl, (C₁₋₃)fluoroalkyl, cycloalkyl, and R₁₀connected to R₆ or R₁₁ as alkyl to form a (C₁₋₃)alkyl bridge cyclic structure;
R₁₂ is selected from the group consisting of H, F, CH₃, alkyl, substituted alkyl, (C₁₋₃)fluoroalkyl, cycloalkyl, and R₁₂ connected to R₉ as alkyl to form a (C₁₋₃)alkyl bridge cyclic structure;
X is selected from the group consisting of absent to optionally provide five membered pyrrolidine ring, CH₂, O, and CRₐR_{b} where Rₐ and R_{b} are selected from the group consisting of alkyl, cycloalkyl, and Fluoroalkyl; and wherein:
the carbon atom at position 2 of the piperidine or pyrrolidine is optionally in absolute (S)-configuration; or,
the carbon atom at position 2 of the of the morpholine ring wherein X is oxygen and optionally in absolute (R)-configuration;
Y is selected from the group consisting of absent to provide R₅′ attached directly to the carbon bearing Z₁ and Z₂ groups; O; NH; CH₂OR₅′; CH₂; NRₐ wherein Rₐ is selected from the group consisting of alkyl, cycloalkyl, and heteroalkyl; and a 5 or 6-membered heteroaryl selected from the group consisting of pyrrolyl, pyrazolyl, triazolyl, oxazolyl, thiazolyl, and oxadiazolyl; and,
Z₁ and Z₂ are independently selected from the group consisting of H, F, (C₁₋₄)alkyl, (C₁₋₃)fluoroalkyl, (C₁₋₃)fluoroalkoxy, and (C₂₋₇)cycloalkyl;
and preferably:
L is selected from the group consisting of Carbon, CH, CHR₂, CHR₃, CR₂R₃, CR₂, CR₃, CR₄, and, N;
or a pharmaceutically acceptable salt, hydrate, solvate, polymorph, isomer, or combination thereof

5. A compound of claim 1 having Formula I-b4, I-b5, I-b6, II-b4, II-b5, or II-b6: 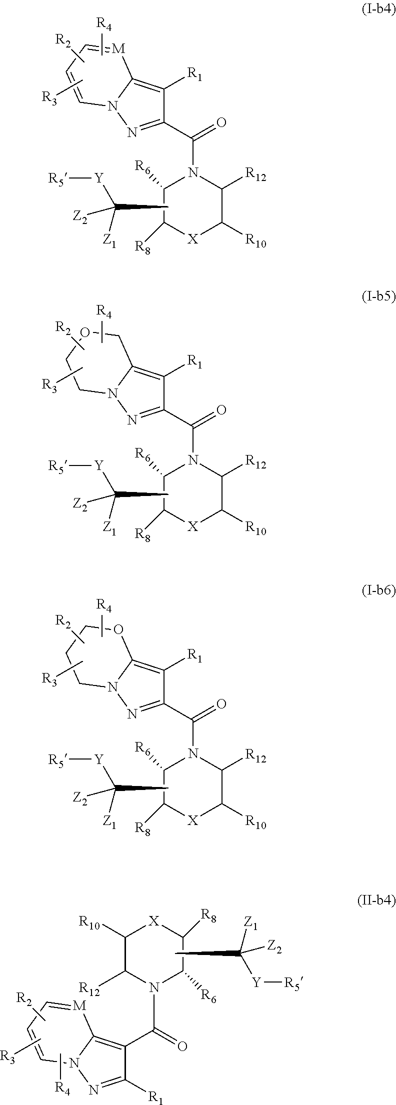 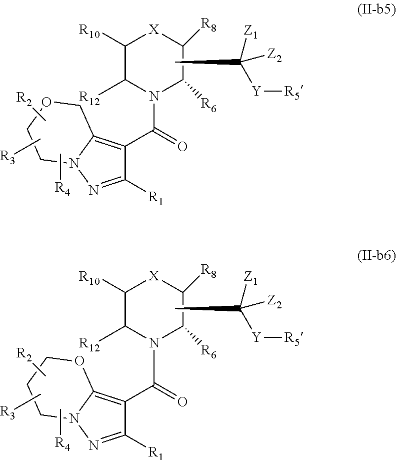
wherein:
R₁ is selected from the group consisting of H, alkyl, alkoxy, cycloalkyl, phenyl, aromatic or aryl, heteroaryl that is optionally a 5- or 6 membered heteroaryl, substituted aromatic or aryl, and, substituted heteroaryl that is optionally a 5- or 6-membered heteroaryl; wherein if R₁ is heteroaryl, R₁ is optionally a 5- or 6-membered heteroaryl selected from the group consisting of pyrazolyl, triazolyl, oxazolyl, thiazolyl, thiophenyl, pyridinyl, pyrimidinyl, pyrazinyl, and pyridazinyl; wherein said aromatic, aryl or heteroaryl is unsubstituted, mono-substituted, or di-substituted, wherein the substituents are independently selected from the group consisting of (C₁₋₄)alkyl, (C₁₋₄)alkoxy, halogen, (C₁₋₃)fluoroalkyl, (C₁₋₃)fluoroalkoxy and (C₃₋₇)cycloalkyl;
R₂, R₃, and R₄ are independently selected from the group consisting of H, halogen (such as F, Cl, Br), alkyl, substituted alkyl, (C₁₋₄)alkyl, (C₁₋₄)alkoxy, (C₁₋₃)fluoroalkyl, (C₁₋₃)fluoroalkoxy and (C₃₋₇)cycloalkyl; wherein each of R₂, R₃ and R₄ is independently and optionally substituted at each substitutable position with up to three (3) substituents independently selected from one, two, or all R₂, R₃ and R₄;
R₅ is H or is replaced with the carbon bearing Y, Z₁ and Z₂, wherein R₅′ is as defined herein;
R₅′ is selected from the group consisting of aromatic, aryl, heteroaryl, 5- or 6-membered heteroaryl, substituted aromatic or aryl, substituted heteroaryl or fused two heteroaryl ring systems, optionally comprising a 5- or 6-membered ring; wherein said aromatic, aryl or heteroaryl is unsubstituted, mono-, or di-substituted or tri-substituted, wherein the substituents are independently selected from the group consisting of (C₁₋₄)alkyl, (C₁₋₄)alkoxy, halogen, (C₁₋₃)fluoroalkyl, (C₁₋₃)fluoroalkoxy, (C₃₋₇)cycloalkyl, and (C₃₋₇)heterocycloalkyl;
R₆ is selected from the group consisting of H, F, CH₃, alkyl, substituted alkyl, (C₁₋₃)fluoroalkyl, cycloalkyl, and R₆ connected to either R₁₀ or R₁₁ as alkyl to form a (C₁₋₃)alkyl bridge cyclic structure;
R₇ is H or is replaced with the carbon bearing Y, Z₁ and Z₂, wherein R₅′ is defined herein;
R₈ is selected from the group consisting of H, F, CH₃, alkyl, substituted alkyl, (C₁₋₃)fluoroalkyl, cycloalkyl, and R₈ connected to either R₁₀, R₁₁ or R₁₂ as alkyl to form a (C₁₋₃)alkyl bridge cyclic structure;
R₉ is selected from the group consisting of H, F, CH₃, alkyl, substituted alkyl, (C₁₋₃)fluoroalkyl, cycloalkyl, and R₉ connected to either R₆ or R₁₂ as alkyl to form a (C₁₋₃)alkyl bridge cyclic structure;
R₁₀ is selected from the group consisting of H, F, CH₃, alkyl, substituted alkyl, (C₁₋₃)fluoroalkyl, cycloalkyl, and R₁₀ connected to R₆ or R₁₁ as alkyl to form a (C₁₋₃)alkyl bridge cyclic structure;
R₁₂ is selected from the group consisting of H, F, CH₃, alkyl, substituted alkyl, (C₁₋₃)fluoroalkyl, cycloalkyl, and R₁₂ connected to R₉ as alkyl to form a (C₁₋₃)alkyl bridge cyclic structure;
X is selected from the group consisting of absent to optionally provide five membered pyrrolidine ring, CH₂, O, and CRₐR_{b} where Rₐ and R_{b} are selected from the group consisting of alkyl, cycloalkyl, and Fluoroalkyl; and wherein:
the carbon atom at position 2 of the piperidine or pyrrolidine ring is optionally in absolute (S)-configuration; or,
the carbon atom at position 2 of the of the morpholine ring wherein X is oxygen and optionally in absolute (R)-configuration;
Y is selected from the group consisting of absent to provide R₅′ attached directly to the carbon bearing Z₁ and Z₂ groups; O; NH; CH₂OR₅′; CH₂; NRₐ wherein Rₐ is selected from the group consisting of alkyl, cycloalkyl, and heteroalkyl; and a 5 or 6-membered heteroaryl selected from the group consisting of pyrrolyl, pyrazolyl, triazolyl, oxazolyl, thiazolyl, and oxadiazolyl; and,
Z₁ and Z₂ are independently selected from the group consisting of H, F, (C₁₋₄)alkyl, (C₁₋₃)fluoroalkyl, (C₁₋₃)fluoroalkoxy, and (C₂₋₇)cycloalkyl;
and preferrably:
M=Carbon, CH, CHR₂, CHR₃, CR₂R₃, CR₂, CR₃, CR₄, N;
or a pharmaceutically acceptable salt, hydrate, solvate, polymorph, isomer, or combination thereof.

6. A compound selected from the group consisting of the compounds presented in Examples 1-263, 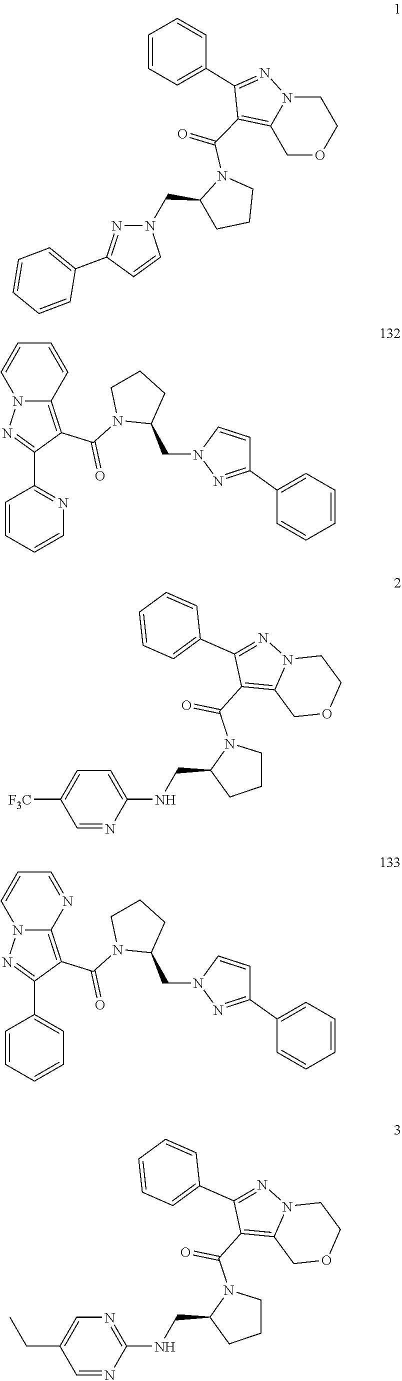 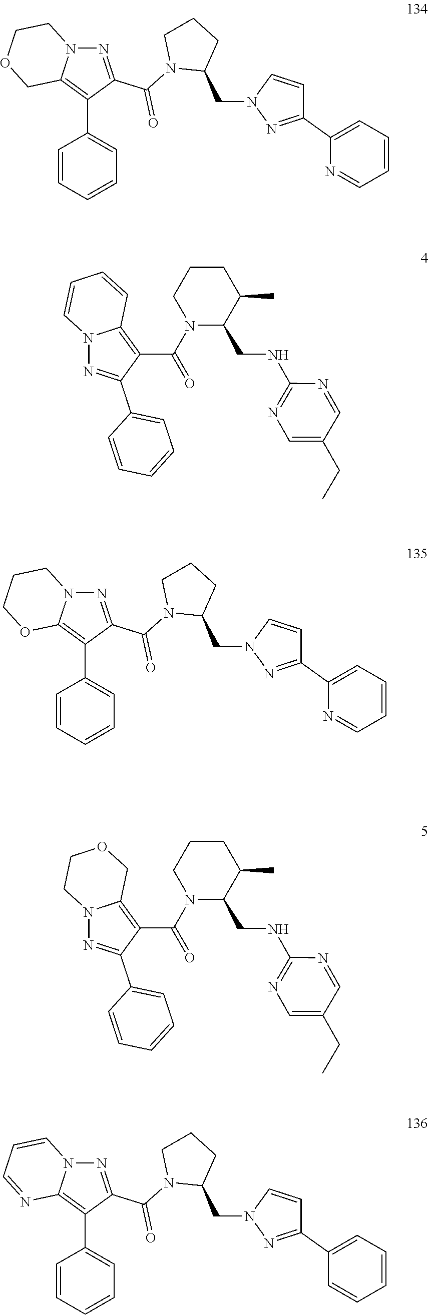 [SMILES: O=C(c1nn2c(c1-c1ccccc1)COCC2)N1CCC[C@H]1Cn1ccc(-c2ccccn2)n1 ; CCc1cnc(NC[C@@H]2[C@H](C)CCCN2C(=O)c2c(-c3ccccc3)nn3ccccc23)nc1 ; O=C(c1nn2c(c1-c1ccccc1)OCCC2)N1CCC[C@H]1Cn1ccc(-c2ccccn2)n1 ; CCc1cnc(NC[C@@H]2[C@H](C)CCCN2C(=O)c2c(-c3ccccc3)nn3c2COCC3)nc1 ; O=C(c1nn2cccnc2c1-c1ccccc1)N1CCC[C@H]1Cn1ccc(-c2ccccc2)n1] 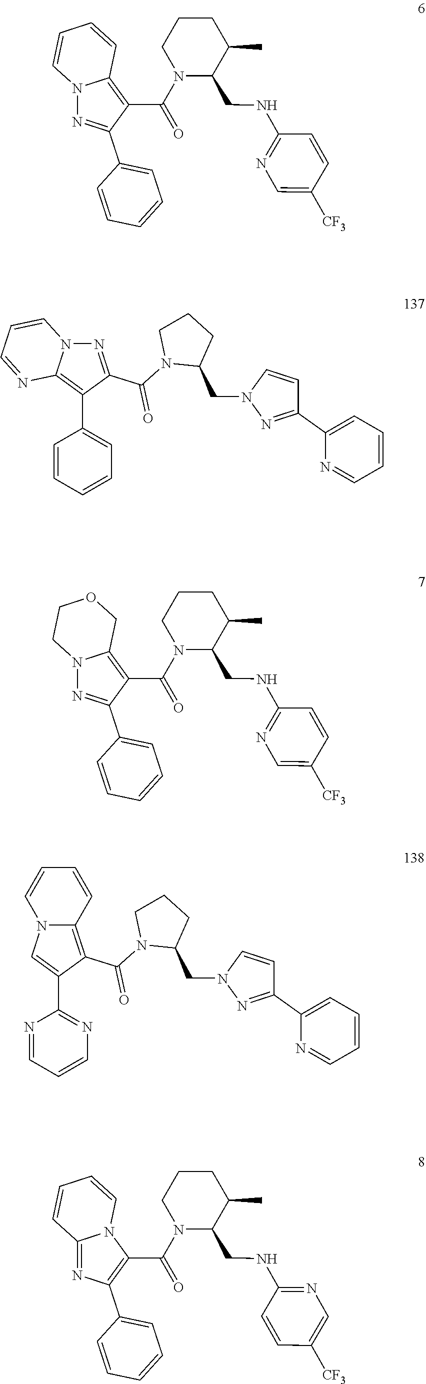 [SMILES: C[C@@H]1CCCN(C(=O)c2c(-c3ccccc3)nn3ccccc23)[C@@H]1CNc1ccc(C(F)(F)F)cn1 ; O=C(c1nn2cccnc2c1-c1ccccc1)N1CCC[C@H]1Cn1ccc(-c2ccccn2)n1 ; C[C@@H]1CCCN(C(=O)c2c(-c3ccccc3)nn3c2COCC3)[C@@H]1CNc1ccc(C(F)(F)F)cn1 ; O=C(c1c(-c2ncccn2)cn2ccccc12)N1CCC[C@H]1Cn1ccc(-c2ccccn2)n1 ; C[C@@H]1CCCN(C(=O)c2c(-c3ccccc3)nc3ccccn23)[C@@H]1CNc1ccc(C(F)(F)F)cn1] 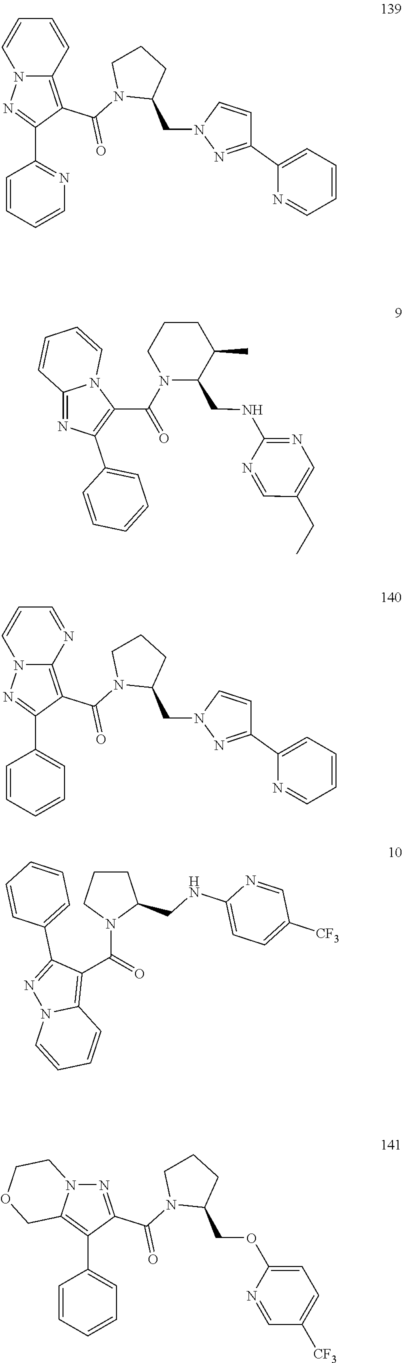 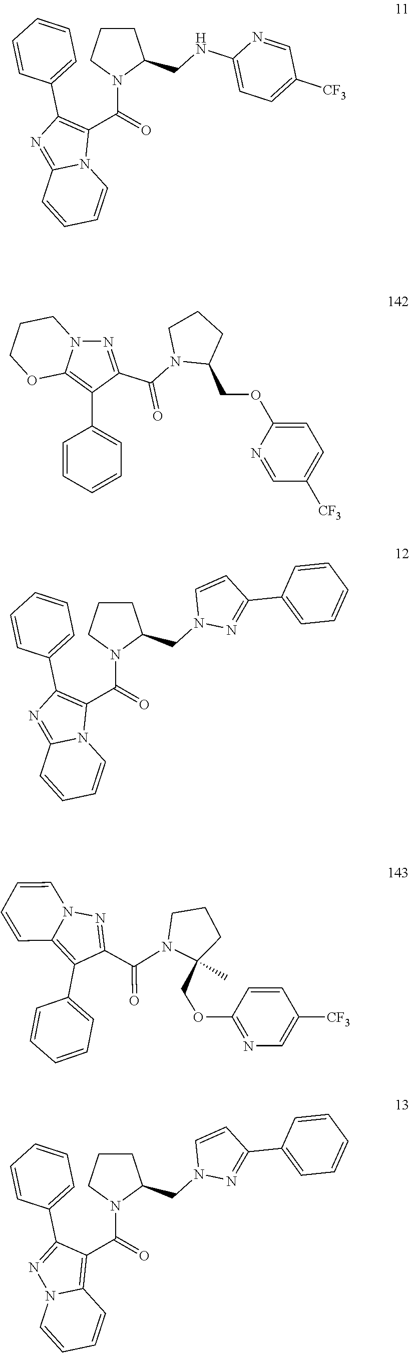 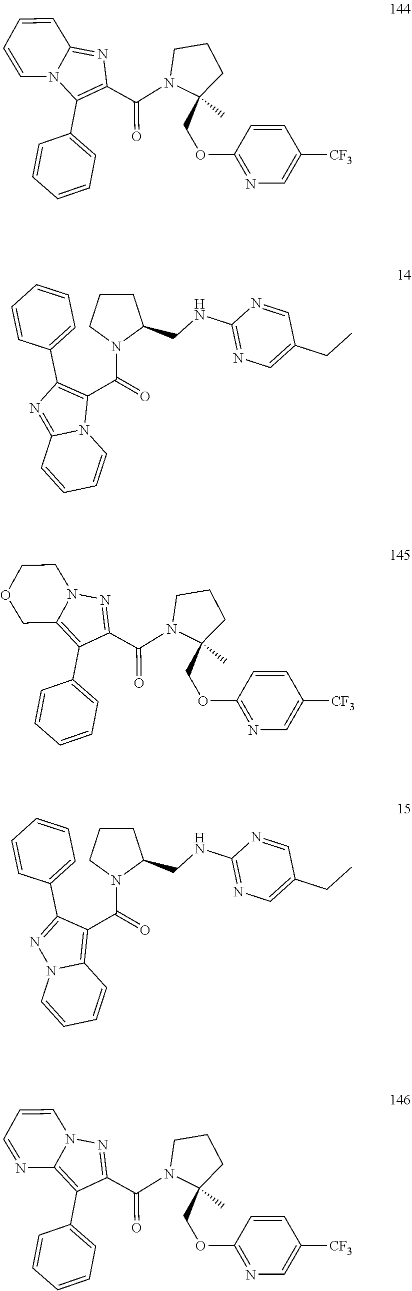 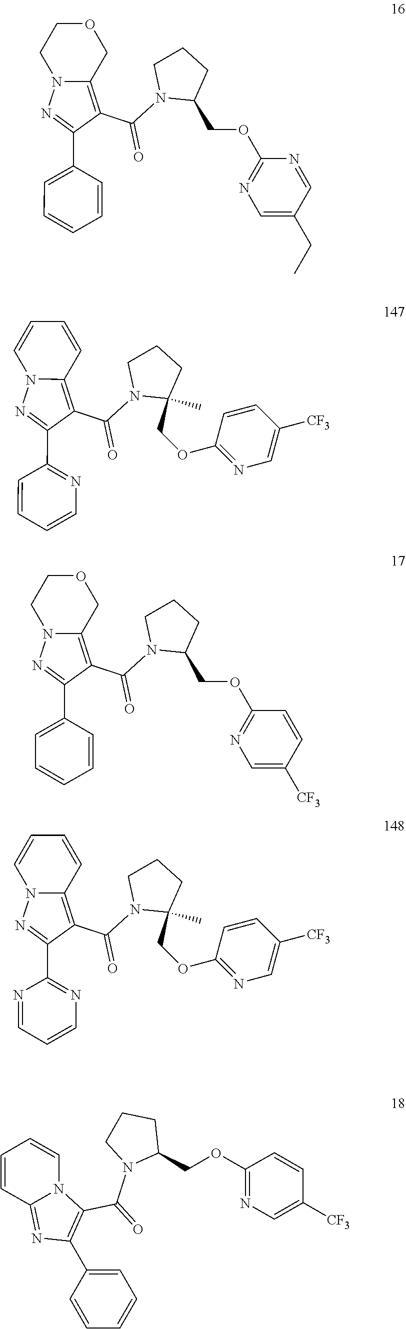 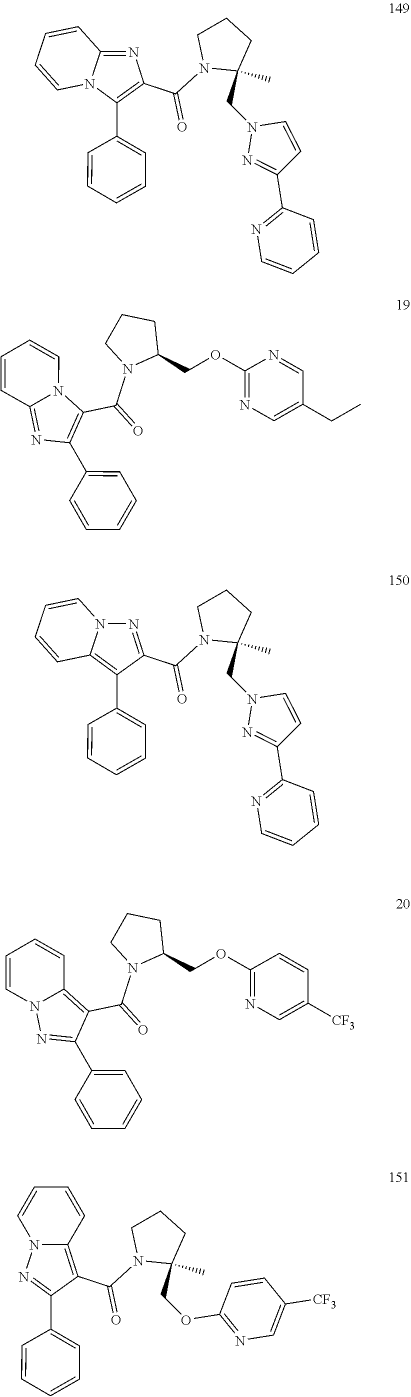 [SMILES: C[C@@]1(Cn2ccc(-c3ccccn3)n2)CCCN1C(=O)c1nc2ccccn2c1-c1ccccc1 ; CCc1cnc(OC[C@@H]2CCCN2C(=O)c2c(-c3ccccc3)nc3ccccn23)nc1 ; C[C@@]1(Cn2ccc(-c3ccccn3)n2)CCCN1C(=O)c1nn2ccccc2c1-c1ccccc1 ; O=C(c1c(-c2ccccc2)nn2ccccc12)N1CCC[C@H]1COc1ccc(C(F)(F)F)cn1 ; C[C@@]1(COc2ccc(C(F)(F)F)cn2)CCCN1C(=O)c1c(-c2ccccc2)nn2ccccc12] 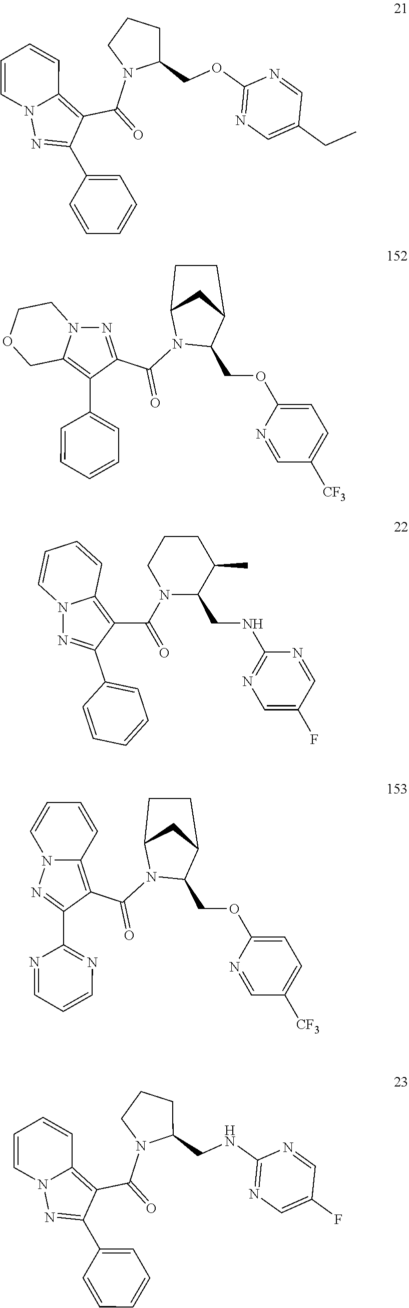 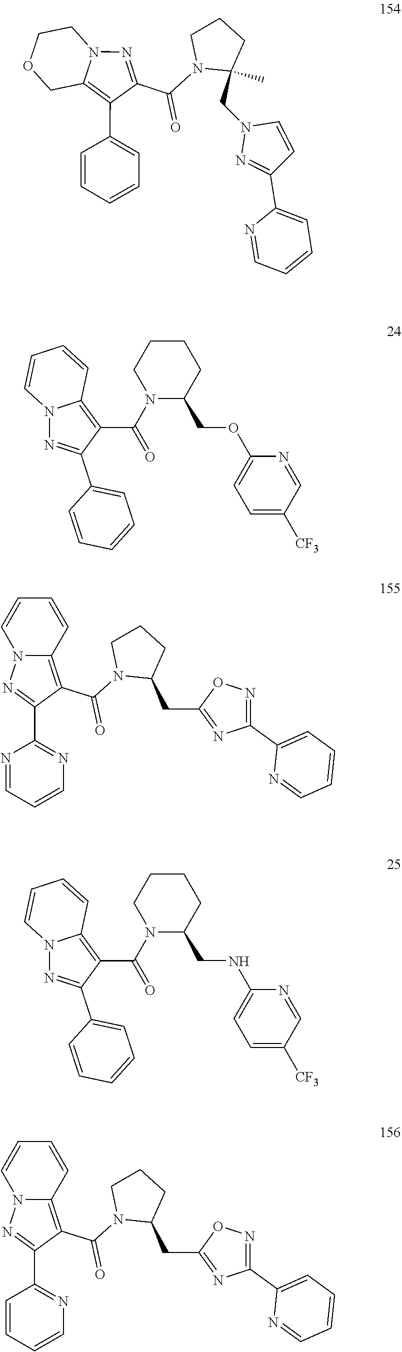 [SMILES: C[C@@]1(Cn2ccc(-c3ccccn3)n2)CCCN1C(=O)c1nn2c(c1-c1ccccc1)COCC2 ; O=C(c1c(-c2ccccc2)nn2ccccc12)N1CCCC[C@H]1COc1ccc(C(F)(F)F)cn1 ; O=C(c1c(-c2ncccn2)nn2ccccc12)N1CCC[C@H]1Cc1nc(-c2ccccn2)no1 ; O=C(c1c(-c2ccccc2)nn2ccccc12)N1CCCC[C@H]1CNc1ccc(C(F)(F)F)cn1 ; O=C(c1c(-c2ccccn2)nn2ccccc12)N1CCC[C@H]1Cc1nc(-c2ccccn2)no1] 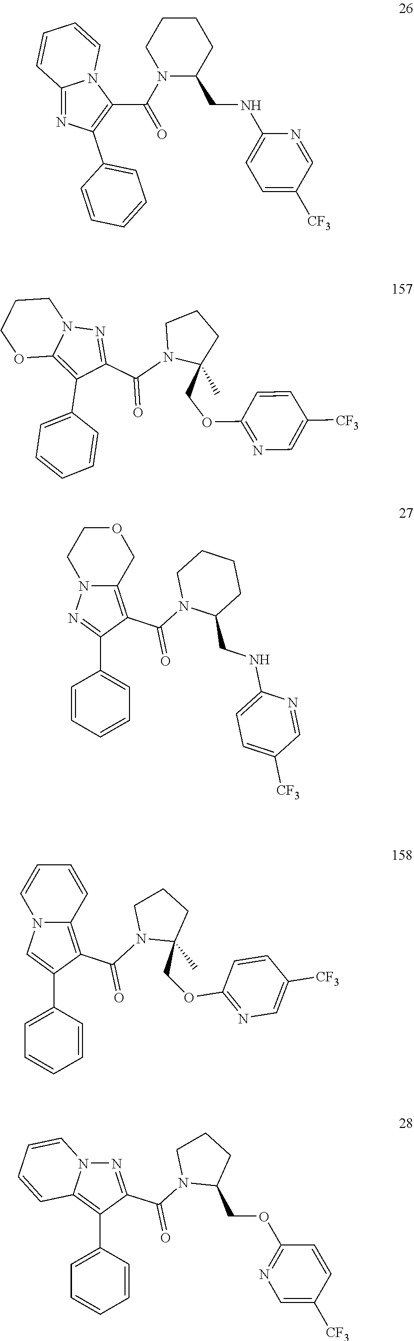 [SMILES: O=C(c1c(-c2ccccc2)nc2ccccn12)N1CCCC[C@H]1CNc1ccc(C(F)(F)F)cn1 ; C[C@@]1(COc2ccc(C(F)(F)F)cn2)CCCN1C(=O)c1nn2c(c1-c1ccccc1)OCCC2 ; O=C(c1c(-c2ccccc2)nn2c1COCC2)N1CCCC[C@H]1CNc1ccc(C(F)(F)F)cn1 ; C[C@@]1(COc2ccc(C(F)(F)F)cn2)CCCN1C(=O)c1c(-c2ccccc2)cn2ccccc12 ; O=C(c1nn2ccccc2c1-c1ccccc1)N1CCC[C@H]1COc1ccc(C(F)(F)F)cn1] 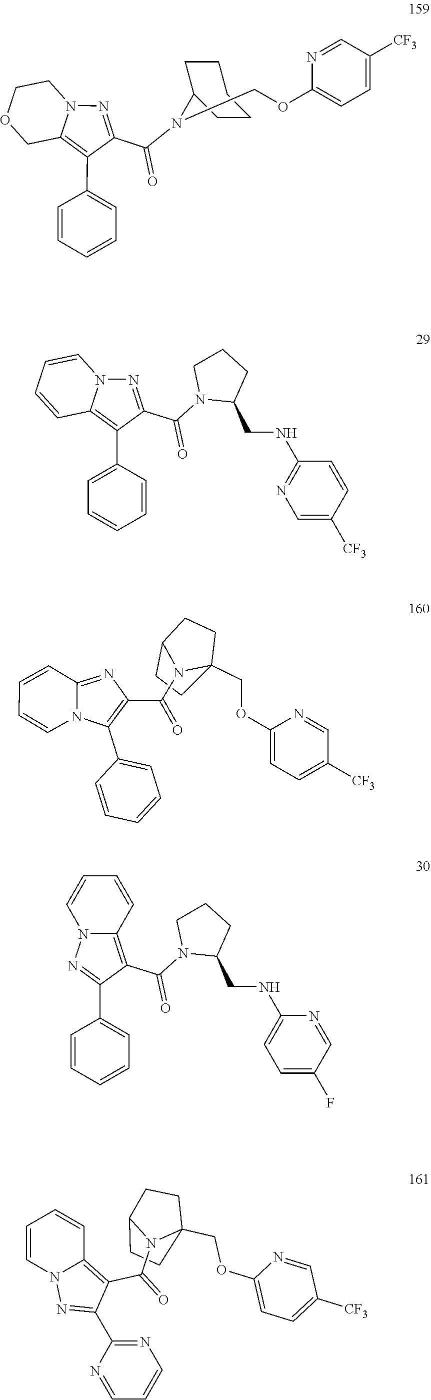 [SMILES: O=C(c1nn2c(c1-c1ccccc1)COCC2)N(COc1ccc(C(F)(F)F)cn1)C1CCCCC1 ; O=C(c1nn2ccccc2c1-c1ccccc1)N1CCC[C@H]1CNc1ccc(C(F)(F)F)cn1 ; O=C(c1nc2ccccn2c1-c1ccccc1)N1C2CCC1(COc1ccc(C(F)(F)F)cn1)CC2 ; O=C(c1c(-c2ccccc2)nn2ccccc12)N1CCC[C@H]1CNc1ccc(F)cn1 ; O=C(c1c(-c2ncccn2)nn2ccccc12)N1C2CCC1(COc1ccc(C(F)(F)F)cn1)CC2] 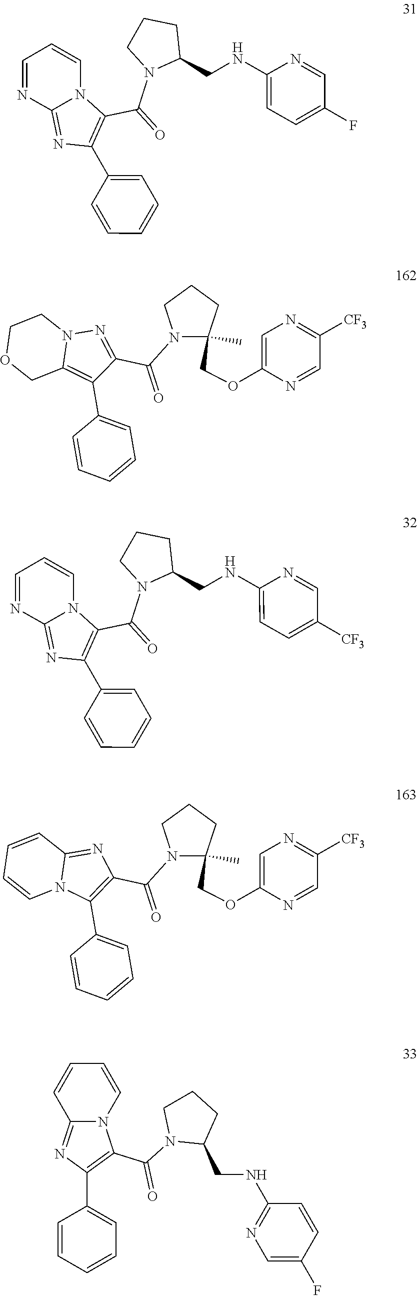 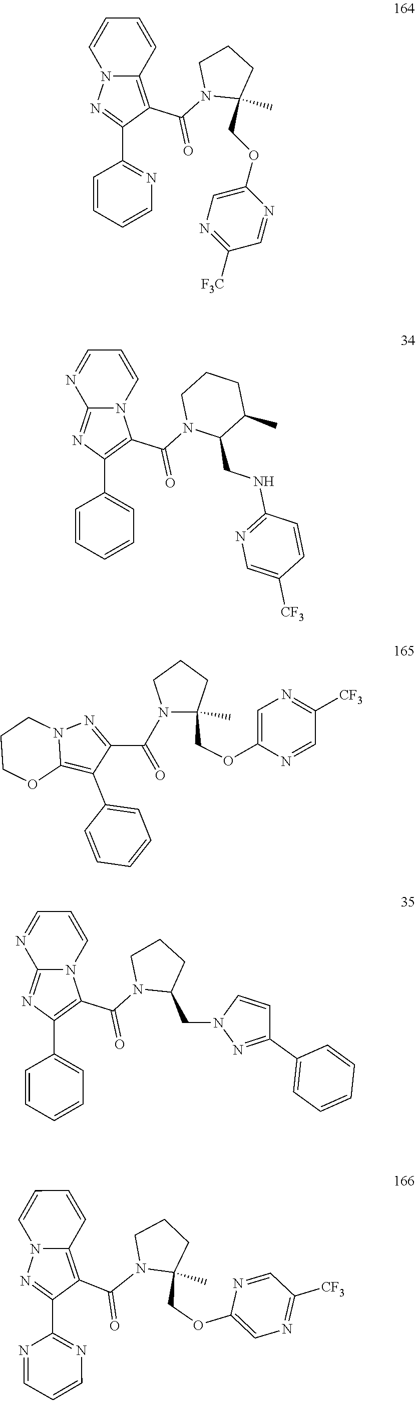 [SMILES: C[C@@]1(COc2cnc(C(F)(F)F)cn2)CCCN1C(=O)c1c(-c2ccccn2)nn2ccccc12 ; C[C@@H]1CCCN(C(=O)c2c(-c3ccccc3)nc3ncccn23)[C@@H]1CNc1ccc(C(F)(F)F)cn1 ; C[C@@]1(COc2cnc(C(F)(F)F)cn2)CCCN1C(=O)c1nn2c(c1-c1ccccc1)OCCC2 ; O=C(c1c(-c2ccccc2)nc2ncccn12)N1CCC[C@H]1Cn1ccc(-c2ccccc2)n1 ; C[C@@]1(COc2cnc(C(F)(F)F)cn2)CCCN1C(=O)c1c(-c2ncccn2)nn2ccccc12] 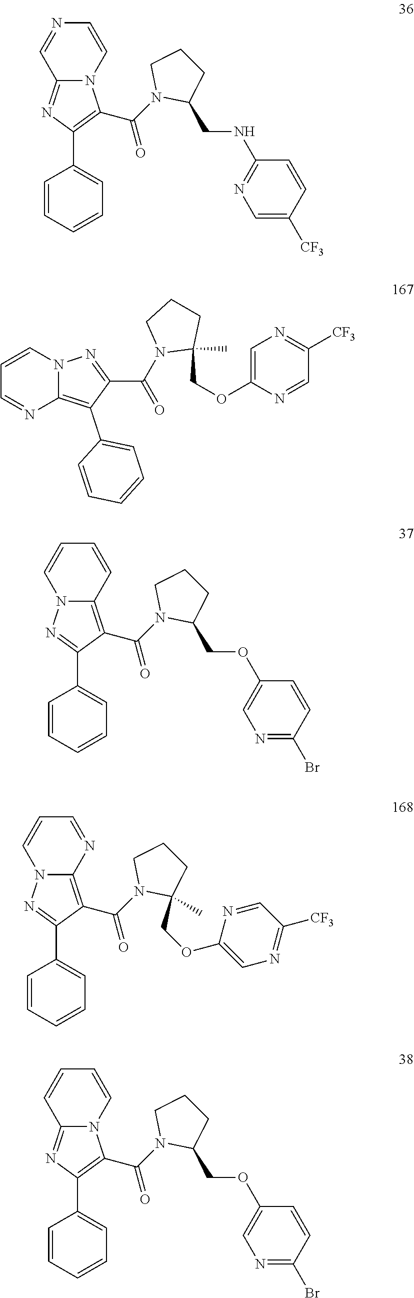 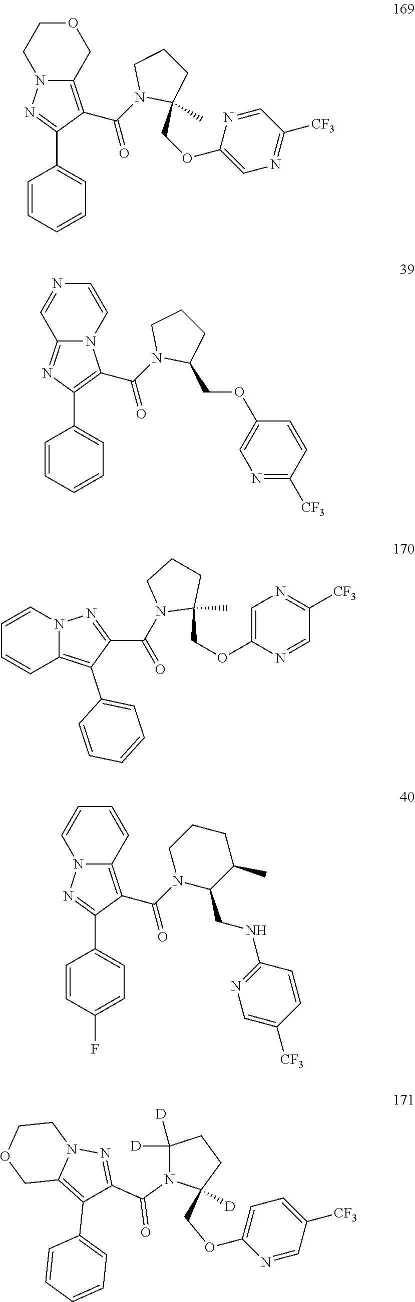 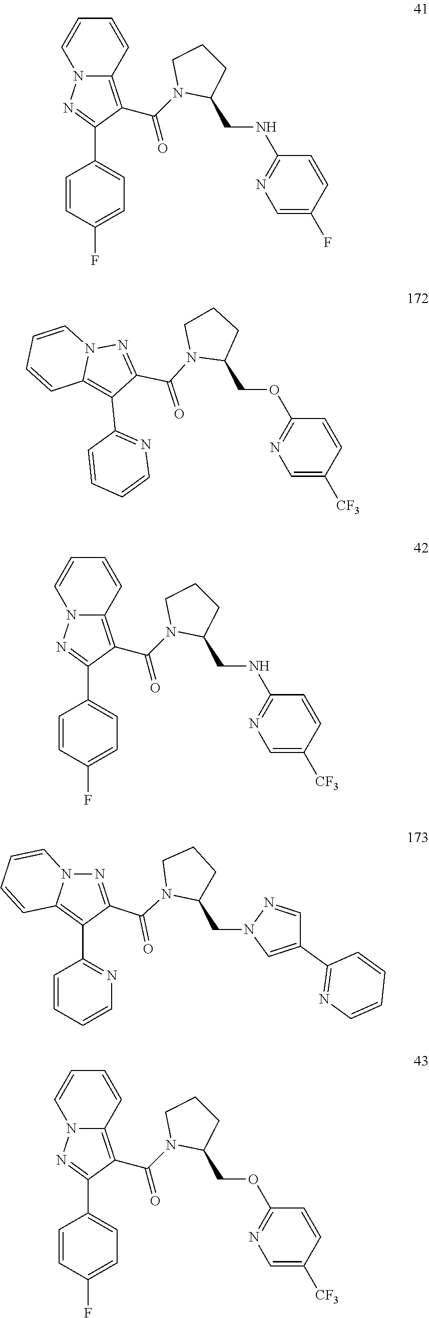 [SMILES: O=C(c1c(-c2ccc(F)cc2)nn2ccccc12)N1CCC[C@H]1CNc1ccc(F)cn1 ; O=C(c1nn2ccccc2c1-c1ccccn1)N1CCC[C@H]1COc1ccc(C(F)(F)F)cn1 ; O=C(c1c(-c2ccc(F)cc2)nn2ccccc12)N1CCC[C@H]1CNc1ccc(C(F)(F)F)cn1 ; O=C(c1nn2ccccc2c1-c1ccccn1)N1CCC[C@H]1Cn1cc(-c2ccccn2)cn1 ; O=C(c1c(-c2ccc(F)cc2)nn2ccccc12)N1CCC[C@H]1COc1ccc(C(F)(F)F)cn1] 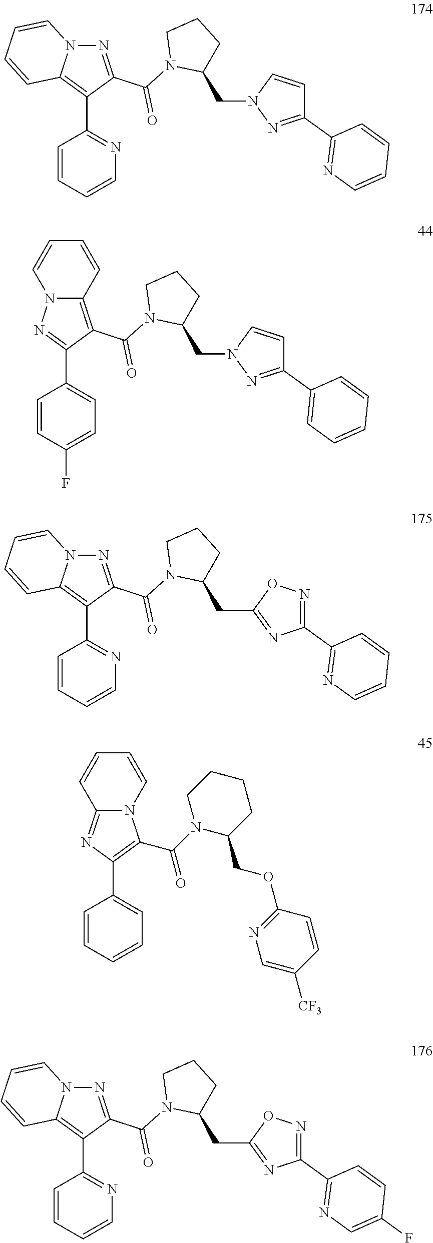 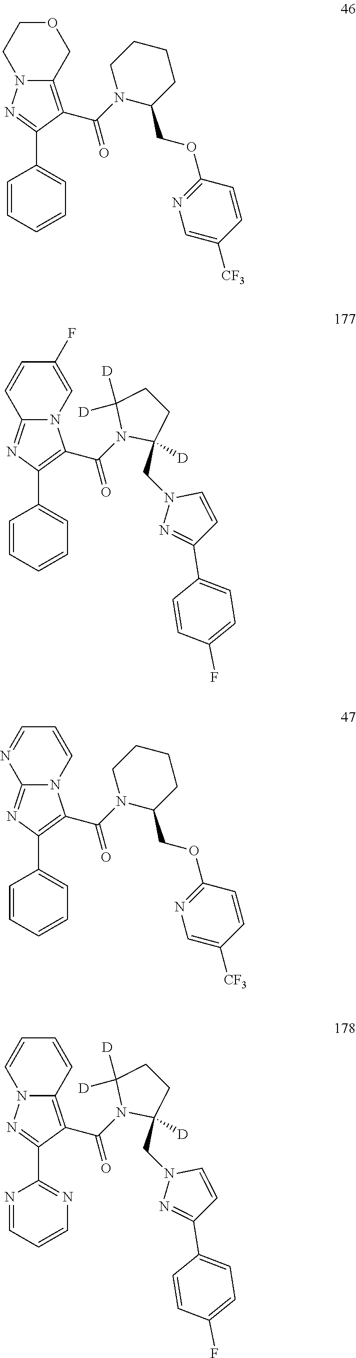 [SMILES: O=C(c1c(-c2ccccc2)nn2c1COCC2)N1CCCC[C@H]1COc1ccc(C(F)(F)F)cn1 ; [2H]C1([2H])CC[C@@]([2H])(Cn2ccc(-c3ccc(F)cc3)n2)N1C(=O)c1c(-c2ccccc2)nc2ccc(F)cn12 ; O=C(c1c(-c2ccccc2)nc2ncccn12)N1CCCC[C@H]1COc1ccc(C(F)(F)F)cn1 ; [2H]C1([2H])CC[C@@]([2H])(Cn2ccc(-c3ccc(F)cc3)n2)N1C(=O)c1c(-c2ncccn2)nn2ccccc12] 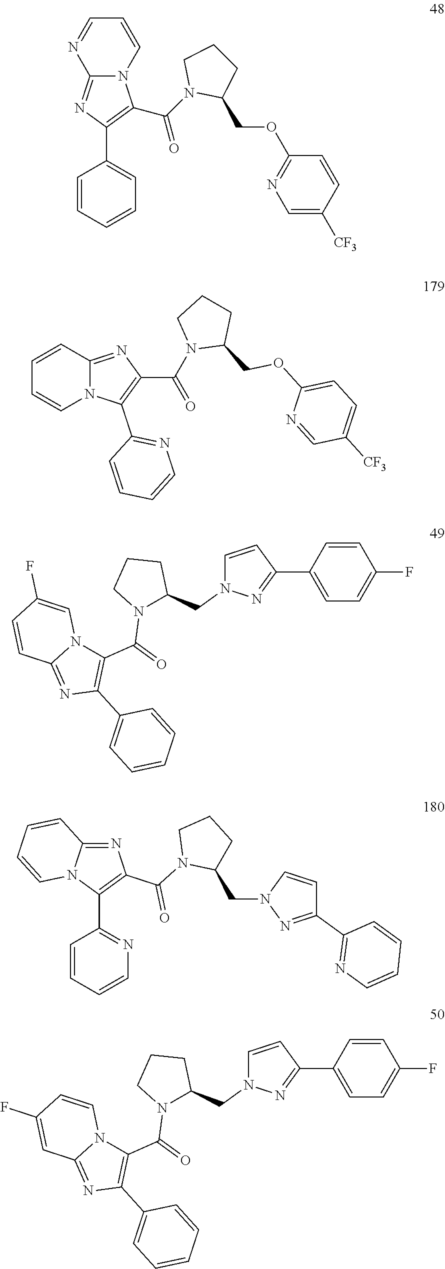 [SMILES: O=C(c1c(-c2ccccc2)nc2ncccn12)N1CCC[C@H]1COc1ccc(C(F)(F)F)cn1 ; O=C(c1nc2ccccn2c1-c1ccccn1)N1CCC[C@H]1COc1ccc(C(F)(F)F)cn1 ; O=C(c1c(-c2ccccc2)nc2ccc(F)cn12)N1CCC[C@H]1Cn1ccc(-c2ccc(F)cc2)n1 ; O=C(c1nc2ccccn2c1-c1ccccn1)N1CCC[C@H]1Cn1ccc(-c2ccccn2)n1 ; O=C(c1c(-c2ccccc2)nc2cc(F)ccn12)N1CCC[C@H]1Cn1ccc(-c2ccc(F)cc2)n1] 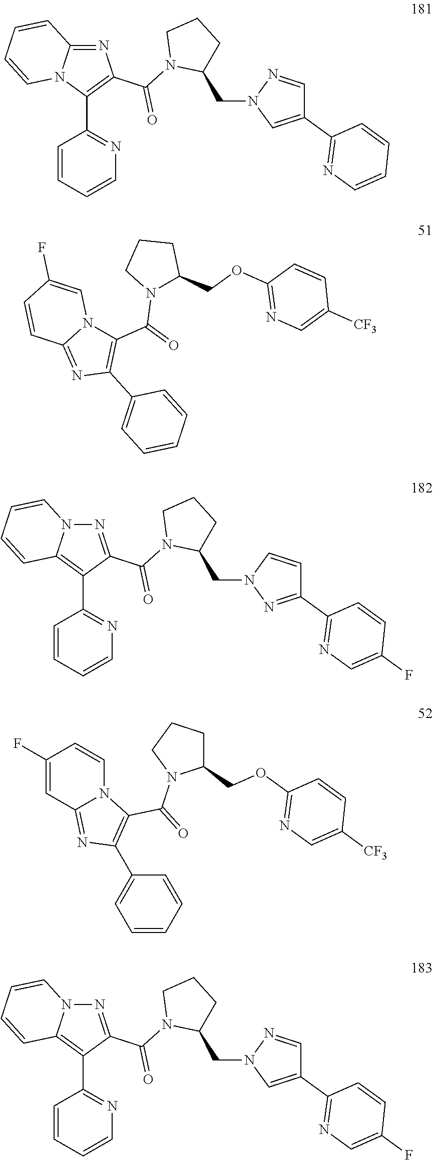 [SMILES: O=C(c1nc2ccccn2c1-c1ccccn1)N1CCC[C@H]1Cn1cc(-c2ccccn2)cn1 ; O=C(c1c(-c2ccccc2)nc2ccc(F)cn12)N1CCC[C@H]1COc1ccc(C(F)(F)F)cn1 ; O=C(c1nn2ccccc2c1-c1ccccn1)N1CCC[C@H]1Cn1ccc(-c2ccc(F)cn2)n1 ; O=C(c1c(-c2ccccc2)nc2cc(F)ccn12)N1CCC[C@H]1COc1ccc(C(F)(F)F)cn1 ; O=C(c1nn2ccccc2c1-c1ccccn1)N1CCC[C@H]1Cn1cc(-c2ccc(F)cn2)cn1] 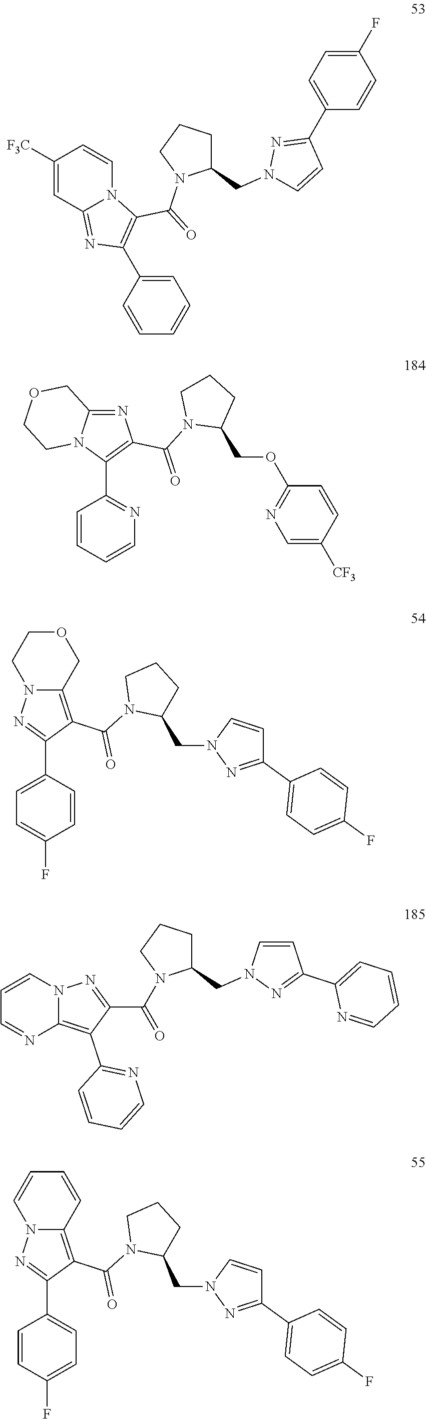 [SMILES: O=C(c1c(-c2ccccc2)nc2cc(C(F)(F)F)ccn12)N1CCC[C@H]1Cn1ccc(-c2ccc(F)cc2)n1 ; O=C(c1nc2n(c1-c1ccccn1)CCOC2)N1CCC[C@H]1COc1ccc(C(F)(F)F)cn1 ; O=C(c1c(-c2ccc(F)cc2)nn2c1COCC2)N1CCC[C@H]1Cn1ccc(-c2ccc(F)cc2)n1 ; O=C(c1nn2cccnc2c1-c1ccccn1)N1CCC[C@H]1Cn1ccc(-c2ccccn2)n1 ; O=C(c1c(-c2ccc(F)cc2)nn2ccccc12)N1CCC[C@H]1Cn1ccc(-c2ccc(F)cc2)n1] 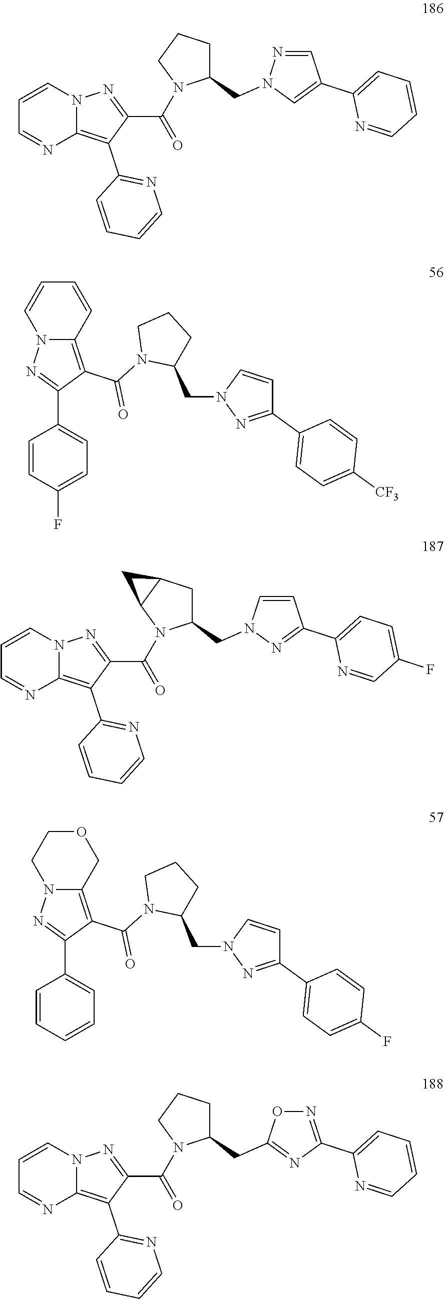 [SMILES: O=C(c1nn2cccnc2c1-c1ccccn1)N1CCC[C@H]1Cn1cc(-c2ccccn2)cn1 ; O=C(c1c(-c2ccc(F)cc2)nn2ccccc12)N1CCC[C@H]1Cn1ccc(-c2ccc(C(F)(F)F)cc2)n1 ; O=C(c1nn2cccnc2c1-c1ccccn1)N1[C@H](Cn2ccc(-c3ccc(F)cn3)n2)C[C@@H]2C[C@@H]21 ; O=C(c1c(-c2ccccc2)nn2c1COCC2)N1CCC[C@H]1Cn1ccc(-c2ccc(F)cc2)n1 ; O=C(c1nn2cccnc2c1-c1ccccn1)N1CCC[C@H]1Cc1nc(-c2ccccn2)no1] 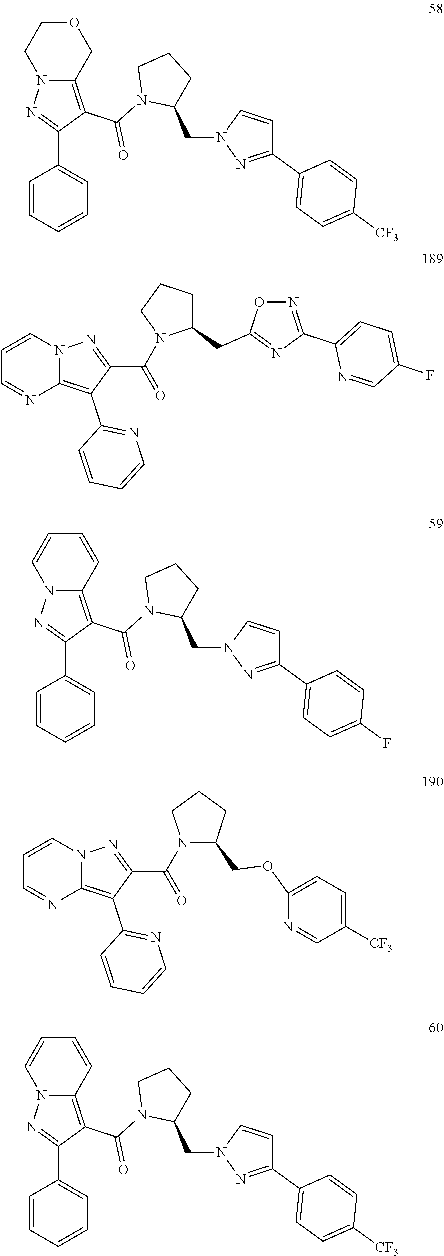 [SMILES: O=C(c1c(-c2ccccc2)nn2c1COCC2)N1CCC[C@H]1Cn1ccc(-c2ccc(C(F)(F)F)cc2)n1 ; O=C(c1nn2cccnc2c1-c1ccccn1)N1CCC[C@H]1Cc1nc(-c2ccc(F)cn2)no1 ; O=C(c1c(-c2ccccc2)nn2ccccc12)N1CCC[C@H]1Cn1ccc(-c2ccc(F)cc2)n1 ; O=C(c1nn2cccnc2c1-c1ccccn1)N1CCC[C@H]1COc1ccc(C(F)(F)F)cn1 ; O=C(c1c(-c2ccccc2)nn2ccccc12)N1CCC[C@H]1Cn1ccc(-c2ccc(C(F)(F)F)cc2)n1] 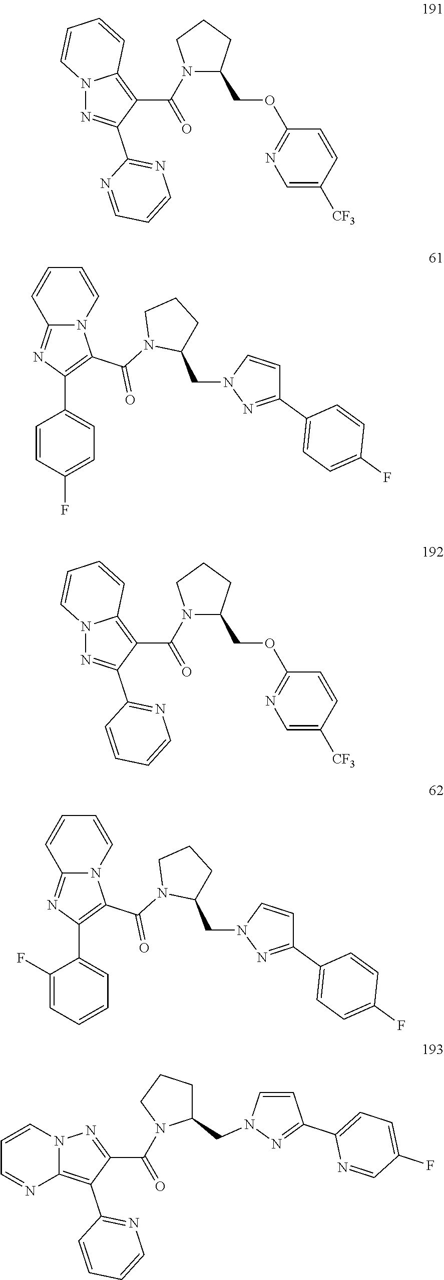 [SMILES: O=C(c1c(-c2ncccn2)nn2ccccc12)N1CCC[C@H]1COc1ccc(C(F)(F)F)cn1 ; O=C(c1c(-c2ccc(F)cc2)nc2ccccn12)N1CCC[C@H]1Cn1ccc(-c2ccc(F)cc2)n1 ; O=C(c1c(-c2ccccn2)nn2ccccc12)N1CCC[C@H]1COc1ccc(C(F)(F)F)cn1 ; O=C(c1c(-c2ccccc2F)nc2ccccn12)N1CCC[C@H]1Cn1ccc(-c2ccc(F)cc2)n1 ; O=C(c1nn2cccnc2c1-c1ccccn1)N1CCC[C@H]1Cn1ccc(-c2ccc(F)cn2)n1] 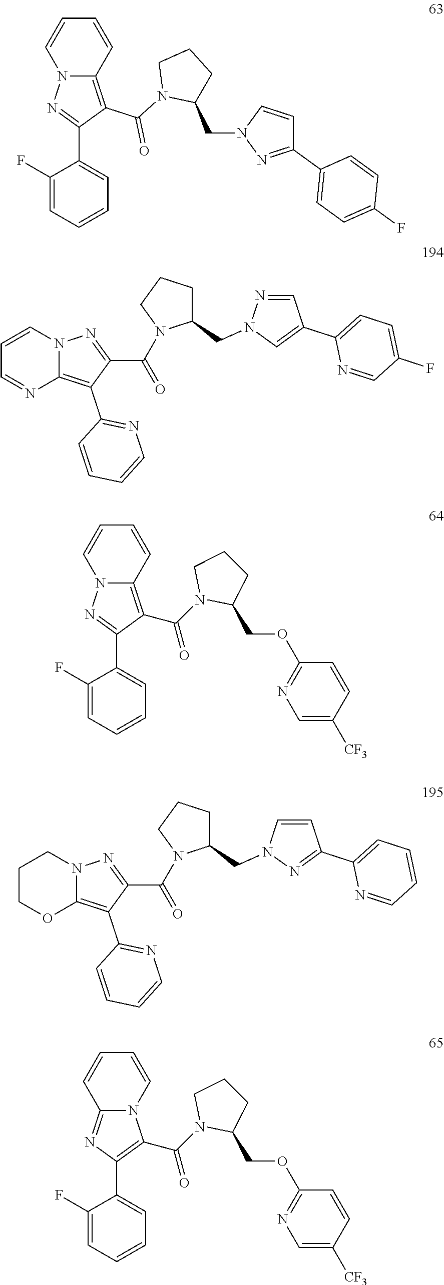 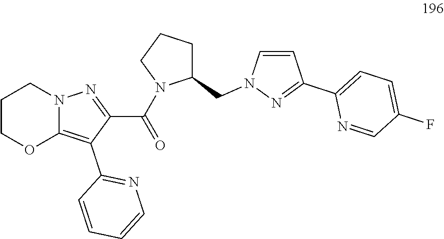 [SMILES: O=C(c1nn2c(c1-c1ccccn1)OCCC2)N1CCC[C@H]1Cn1ccc(-c2ccc(F)cn2)n1] 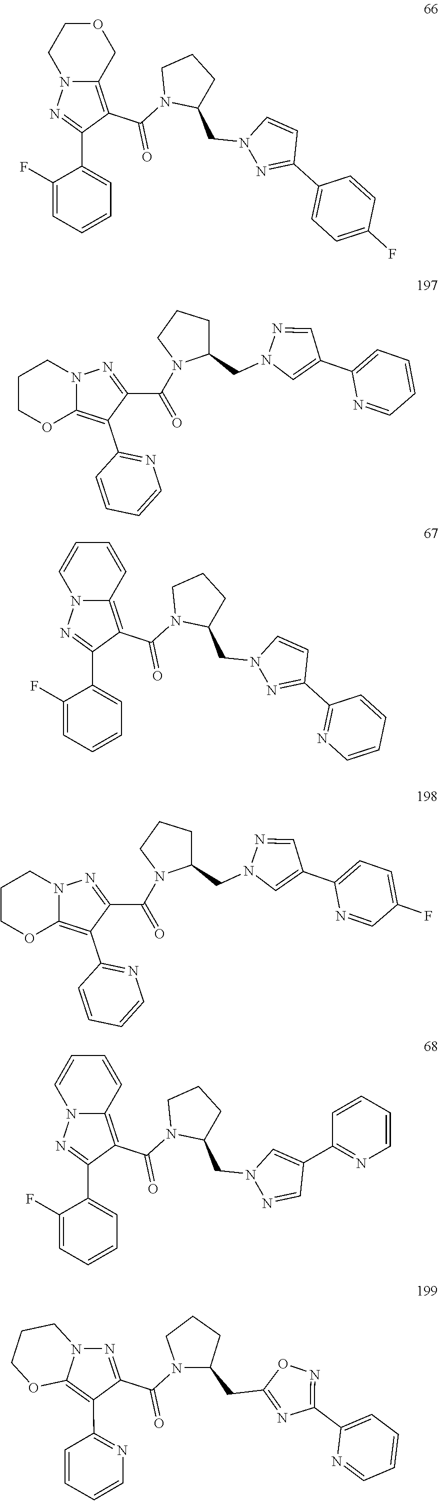 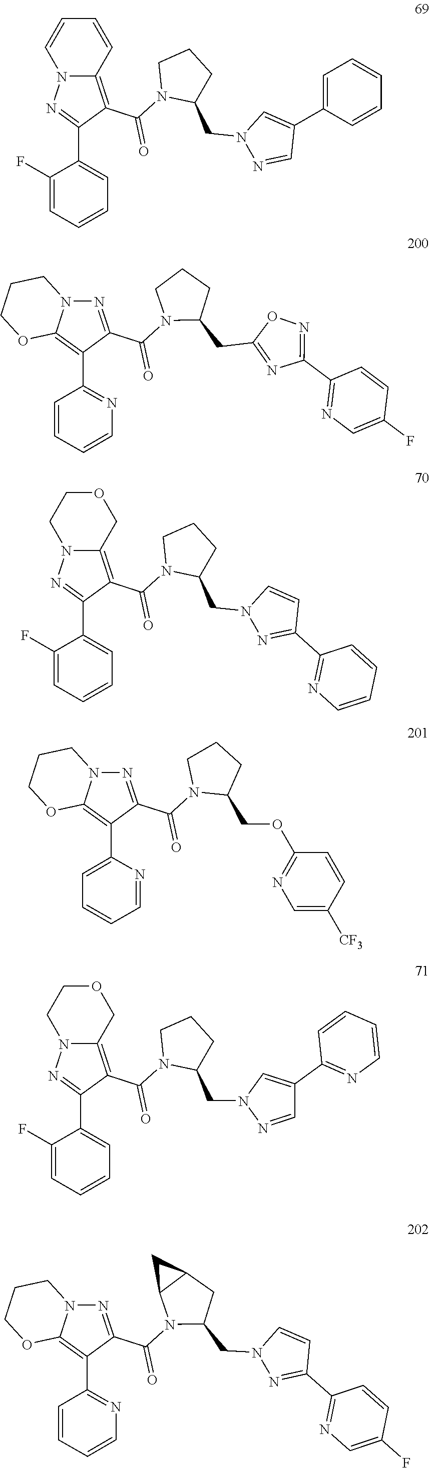 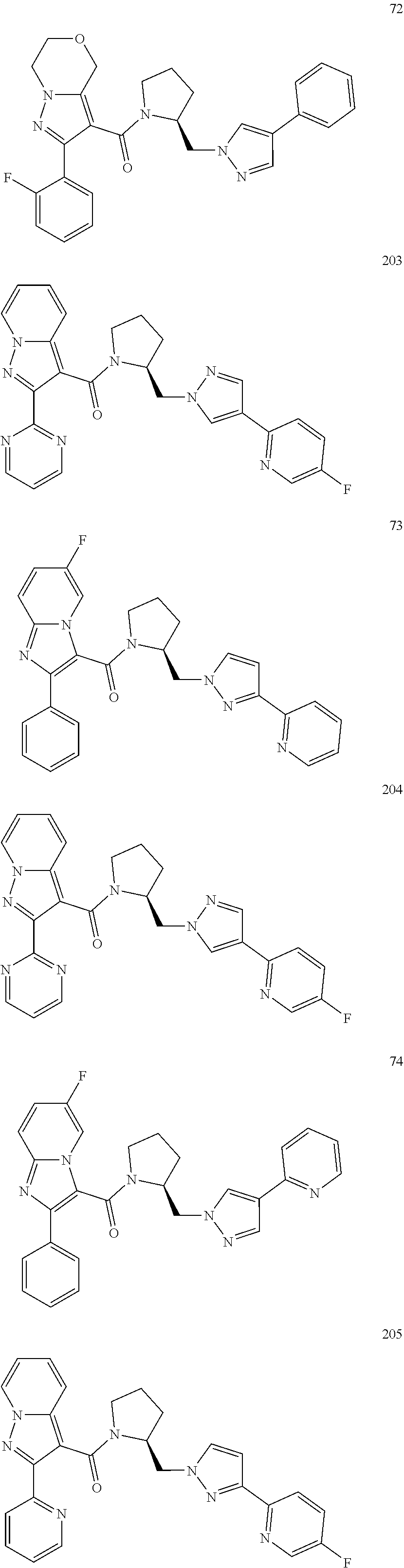 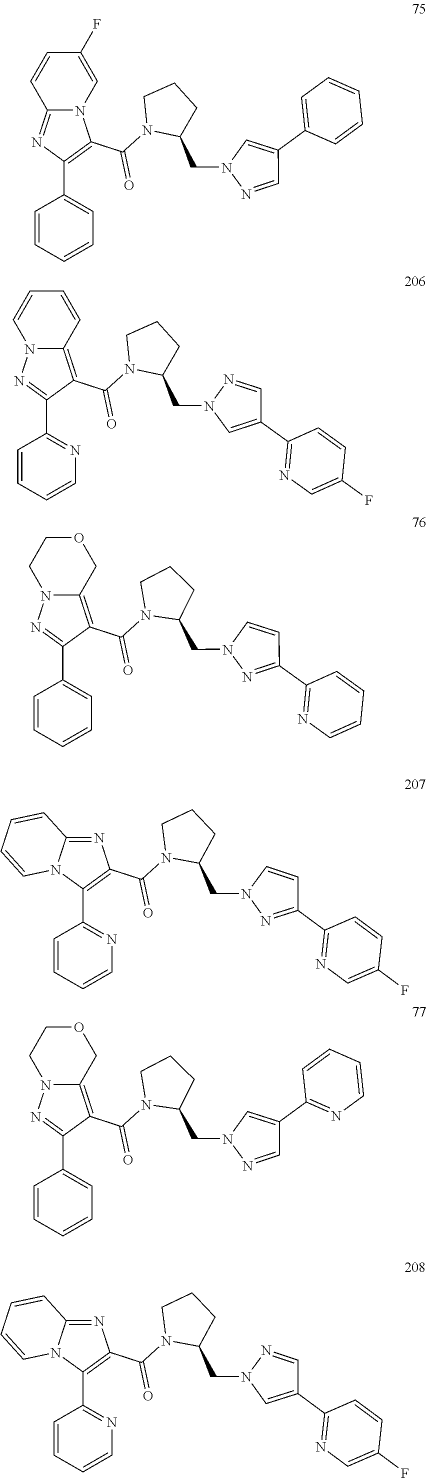 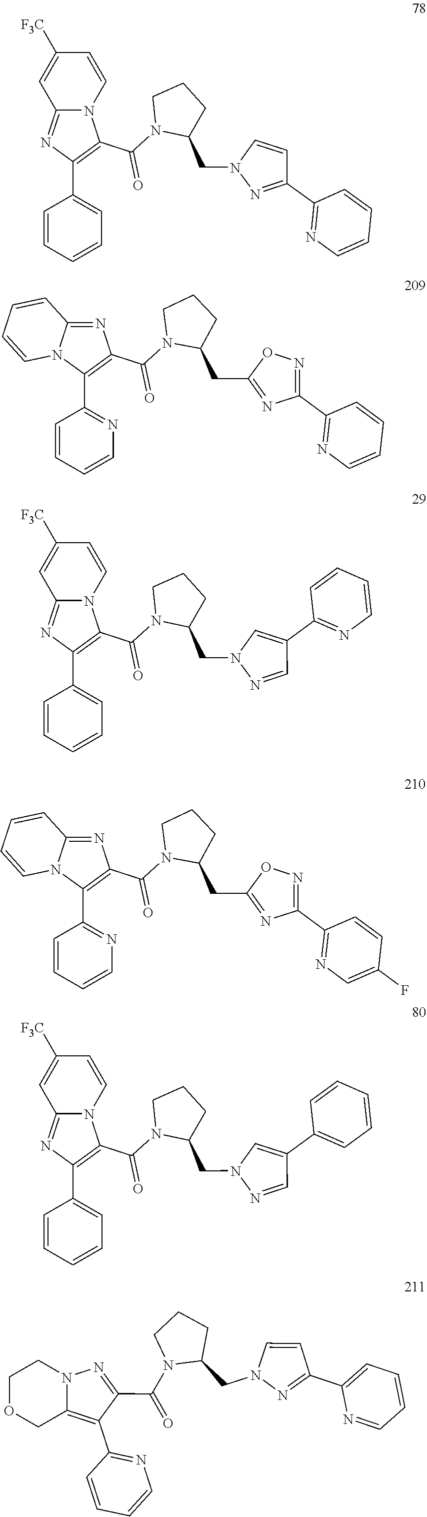 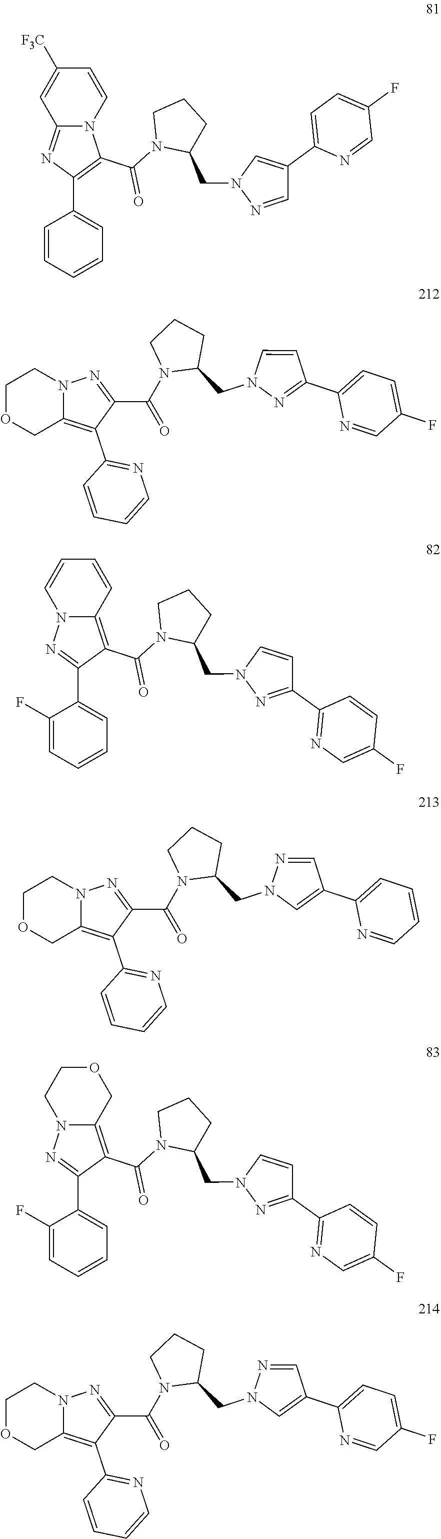 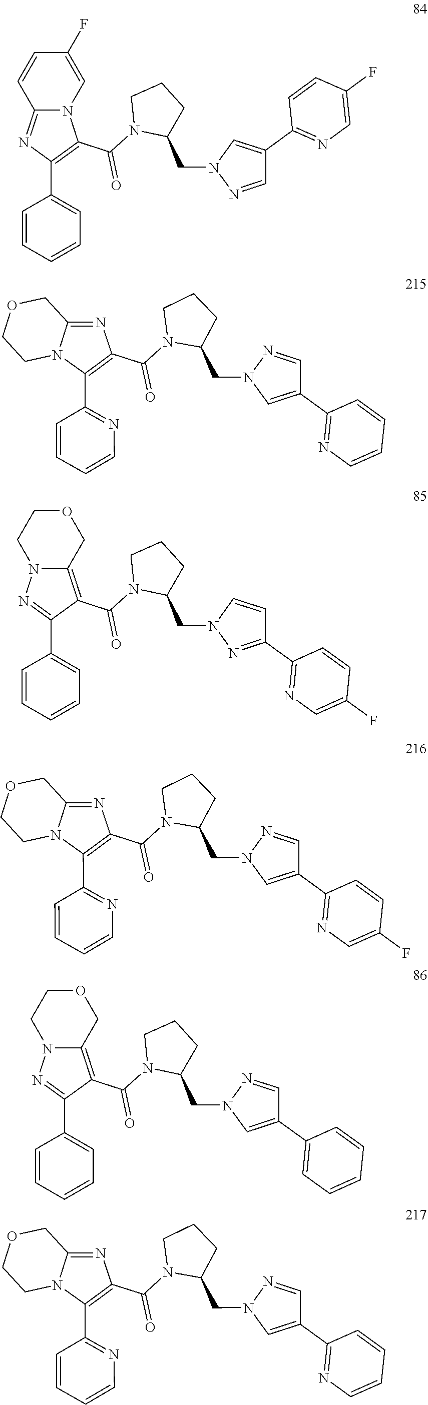 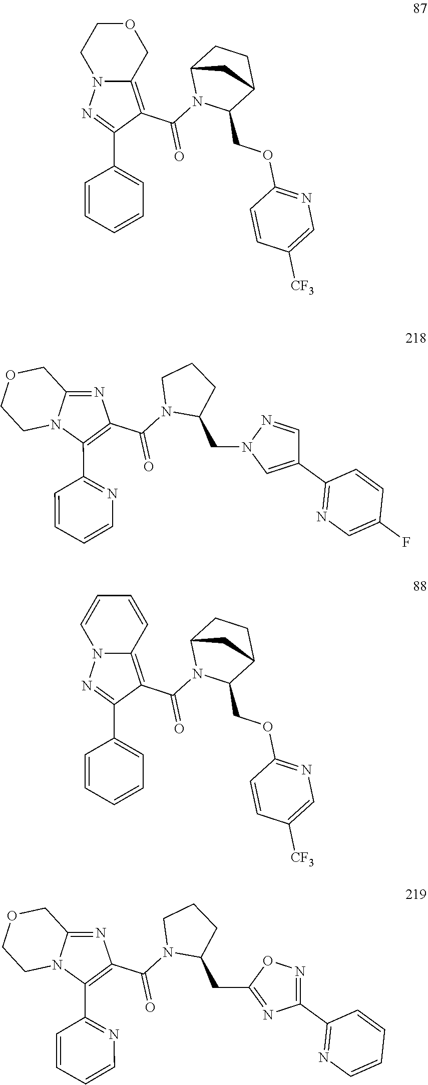 [SMILES: O=C(c1c(-c2ccccc2)nn2c1COCC2)N1[C@@H]2CC[C@@H](C2)[C@H]1COc1ccc(C(F)(F)F)cn1 ; O=C(c1nc2n(c1-c1ccccn1)CCOC2)N1CCC[C@H]1Cn1cc(-c2ccc(F)cn2)cn1 ; O=C(c1c(-c2ccccc2)nn2ccccc12)N1[C@@H]2CC[C@@H](C2)[C@H]1COc1ccc(C(F)(F)F)cn1 ; O=C(c1nc2n(c1-c1ccccn1)CCOC2)N1CCC[C@H]1Cc1nc(-c2ccccn2)no1] 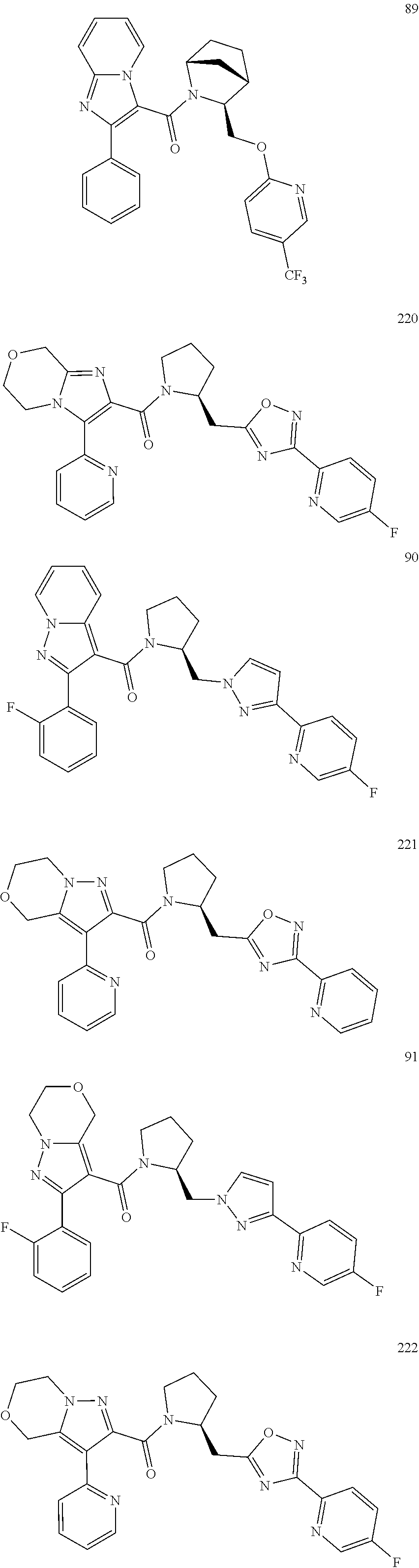 [SMILES: O=C(c1c(-c2ccccc2)nc2ccccn12)N1[C@@H]2CC[C@@H](C2)[C@H]1COc1ccc(C(F)(F)F)cn1 ; O=C(c1nc2n(c1-c1ccccn1)CCOC2)N1CCC[C@H]1Cc1nc(-c2ccc(F)cn2)no1 ; O=C(c1c(-c2ccccc2F)nn2ccccc12)N1CCC[C@H]1Cn1ccc(-c2ccc(F)cn2)n1 ; O=C(c1nn2c(c1-c1ccccn1)COCC2)N1CCC[C@H]1Cc1nc(-c2ccccn2)no1 ; O=C(c1c(-c2ccccc2F)nn2c1COCC2)N1CCC[C@H]1Cn1ccc(-c2ccc(F)cn2)n1 ; O=C(c1nn2c(c1-c1ccccn1)COCC2)N1CCC[C@H]1Cc1nc(-c2ccc(F)cn2)no1] 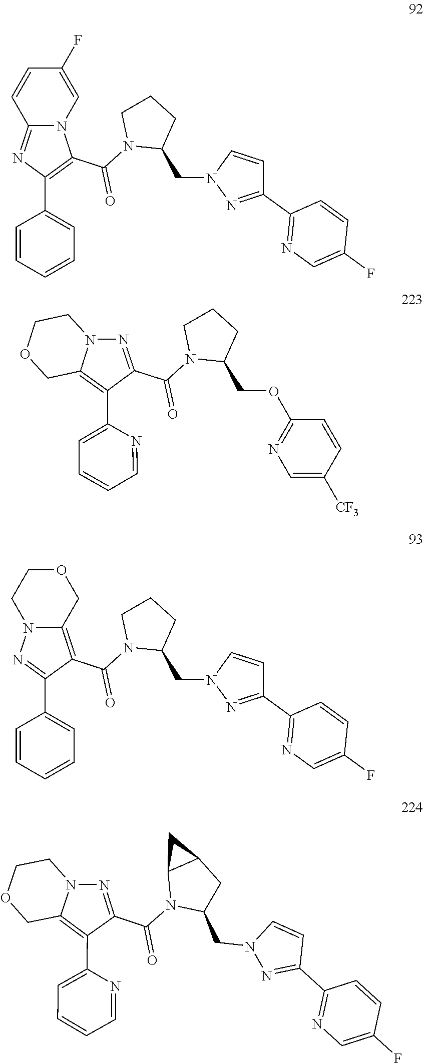 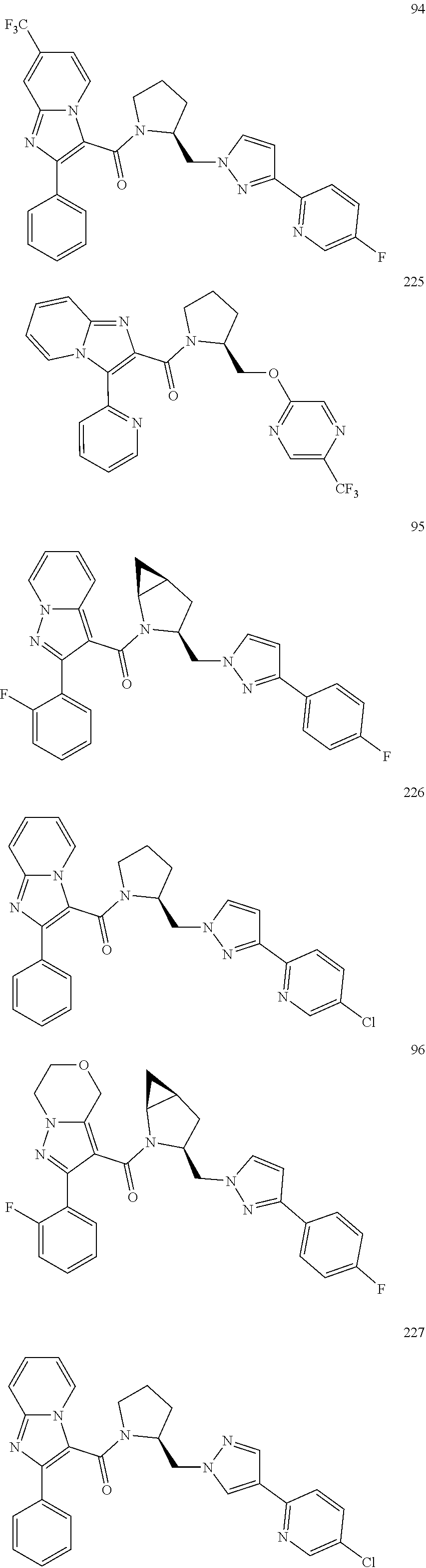 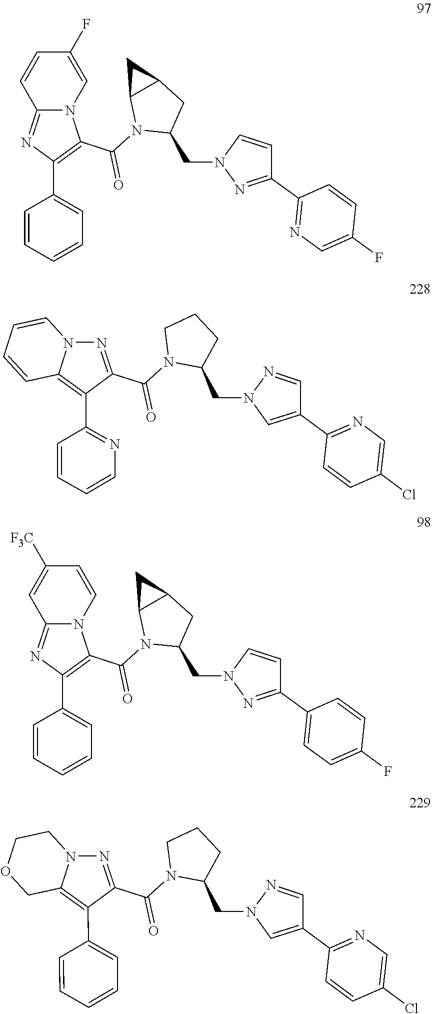 [SMILES: O=C(c1c(-c2ccccc2)nc2ccc(F)cn12)N1[C@H](Cn2ccc(-c3ccc(F)cn3)n2)C[C@@H]2C[C@@H]21 ; O=C(c1nn2ccccc2c1-c1ccccn1)N1CCC[C@H]1Cn1cc(-c2ccc(Cl)cn2)cn1 ; O=C(c1c(-c2ccccc2)nc2cc(C(F)(F)F)ccn12)N1[C@H](Cn2ccc(-c3ccc(F)cc3)n2)C[C@@H]2C[C@@H]21 ; O=C(c1nn2c(c1-c1ccccc1)COCC2)N1CCC[C@H]1Cn1cc(-c2ccc(Cl)cn2)cn1] 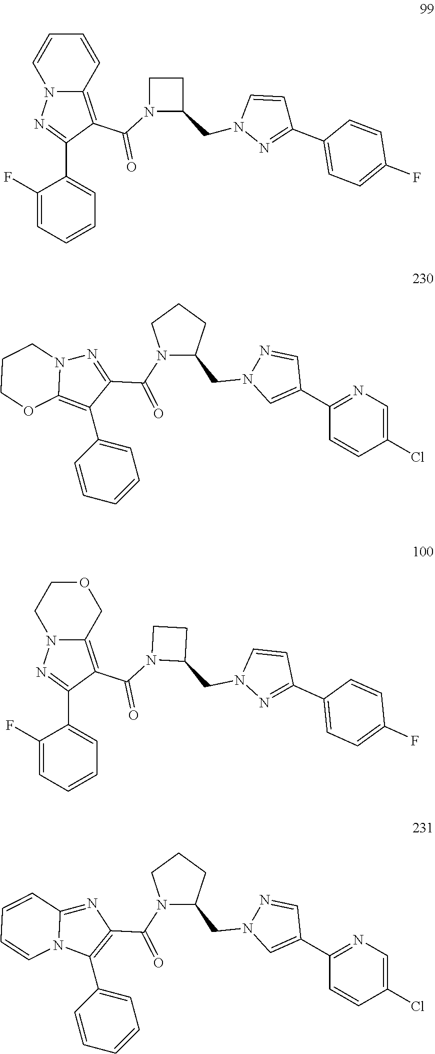 [SMILES: O=C(c1c(-c2ccccc2F)nn2ccccc12)N1CC[C@H]1Cn1ccc(-c2ccc(F)cc2)n1 ; O=C(c1nn2c(c1-c1ccccc1)OCCC2)N1CCC[C@H]1Cn1cc(-c2ccc(Cl)cn2)cn1 ; O=C(c1c(-c2ccccc2F)nn2c1COCC2)N1CC[C@H]1Cn1ccc(-c2ccc(F)cc2)n1 ; O=C(c1nc2ccccn2c1-c1ccccc1)N1CCC[C@H]1Cn1cc(-c2ccc(Cl)cn2)cn1] 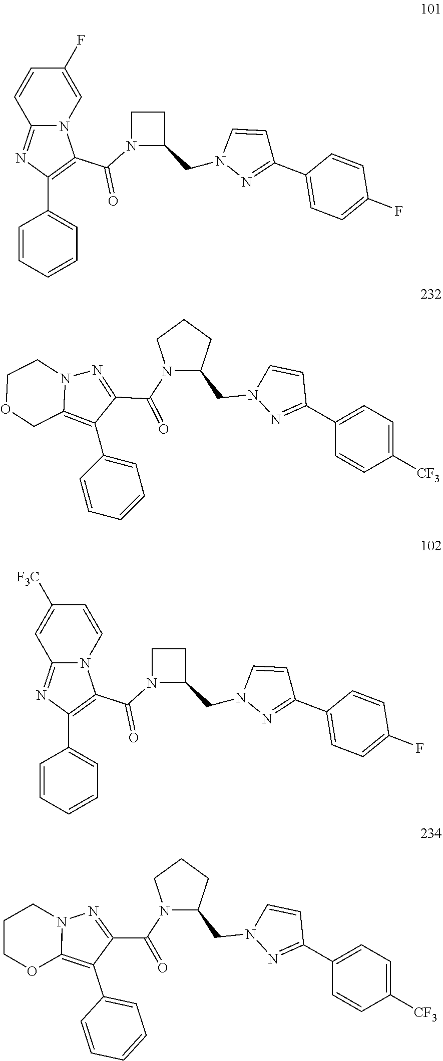 [SMILES: O=C(c1c(-c2ccccc2)nc2ccc(F)cn12)N1CC[C@H]1Cn1ccc(-c2ccc(F)cc2)n1 ; O=C(c1nn2c(c1-c1ccccc1)COCC2)N1CCC[C@H]1Cn1ccc(-c2ccc(C(F)(F)F)cc2)n1 ; O=C(c1c(-c2ccccc2)nc2cc(C(F)(F)F)ccn12)N1CC[C@H]1Cn1ccc(-c2ccc(F)cc2)n1 ; O=C(c1nn2c(c1-c1ccccc1)OCCC2)N1CCC[C@H]1Cn1ccc(-c2ccc(C(F)(F)F)cc2)n1] 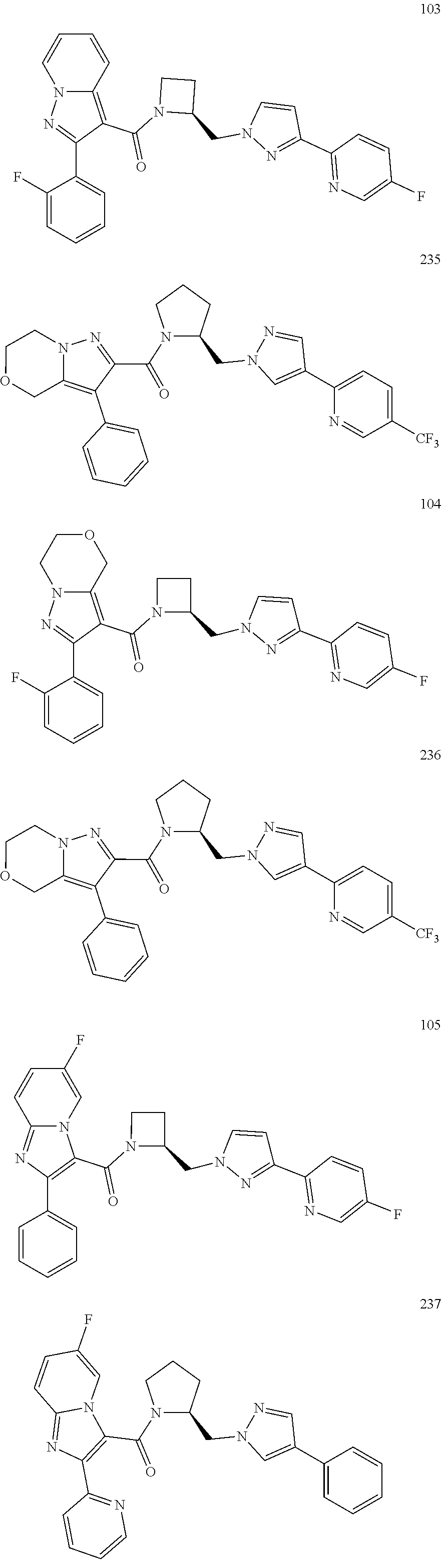 [SMILES: O=C(c1c(-c2ccccc2F)nn2ccccc12)N1CC[C@H]1Cn1ccc(-c2ccc(F)cn2)n1 ; O=C(c1nn2c(c1-c1ccccc1)COCC2)N1CCC[C@H]1Cn1cc(-c2ccc(C(F)(F)F)cn2)cn1 ; O=C(c1c(-c2ccccc2F)nn2c1COCC2)N1CC[C@H]1Cn1ccc(-c2ccc(F)cn2)n1 ; O=C(c1nn2c(c1-c1ccccc1)COCC2)N1CCC[C@H]1Cn1cc(-c2ccc(C(F)(F)F)cn2)cn1 ; O=C(c1c(-c2ccccc2)nc2ccc(F)cn12)N1CC[C@H]1Cn1ccc(-c2ccc(F)cn2)n1 ; O=C(c1c(-c2ccccn2)nc2ccc(F)cn12)N1CCC[C@H]1Cn1cc(-c2ccccc2)cn1] 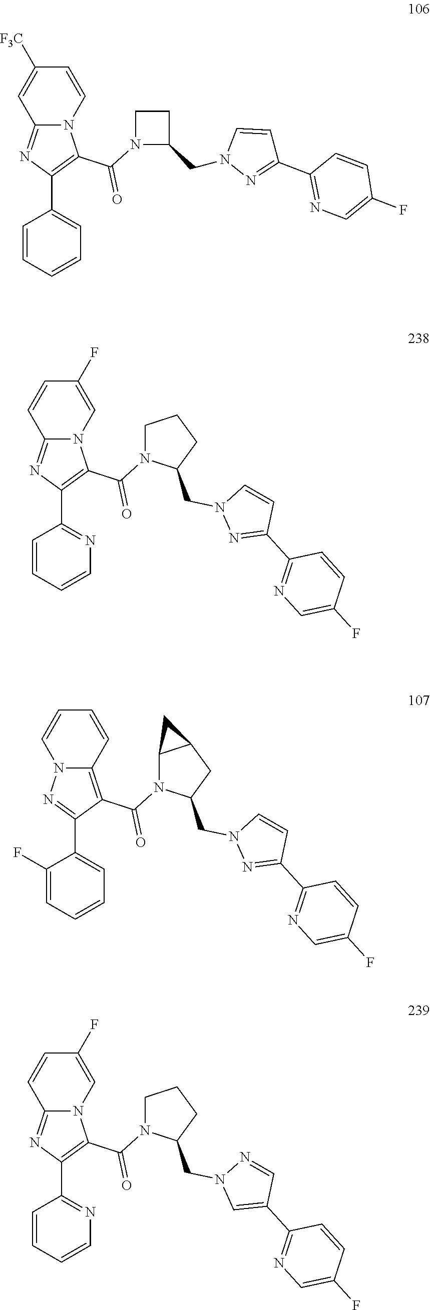 [SMILES: O=C(c1c(-c2ccccc2)nc2cc(C(F)(F)F)ccn12)N1CC[C@H]1Cn1ccc(-c2ccc(F)cn2)n1 ; O=C(c1c(-c2ccccn2)nc2ccc(F)cn12)N1CCC[C@H]1Cn1ccc(-c2ccc(F)cn2)n1 ; O=C(c1c(-c2ccccc2F)nn2ccccc12)N1[C@H](Cn2ccc(-c3ccc(F)cn3)n2)C[C@@H]2C[C@@H]21 ; O=C(c1c(-c2ccccn2)nc2ccc(F)cn12)N1CCC[C@H]1Cn1cc(-c2ccc(F)cn2)cn1] 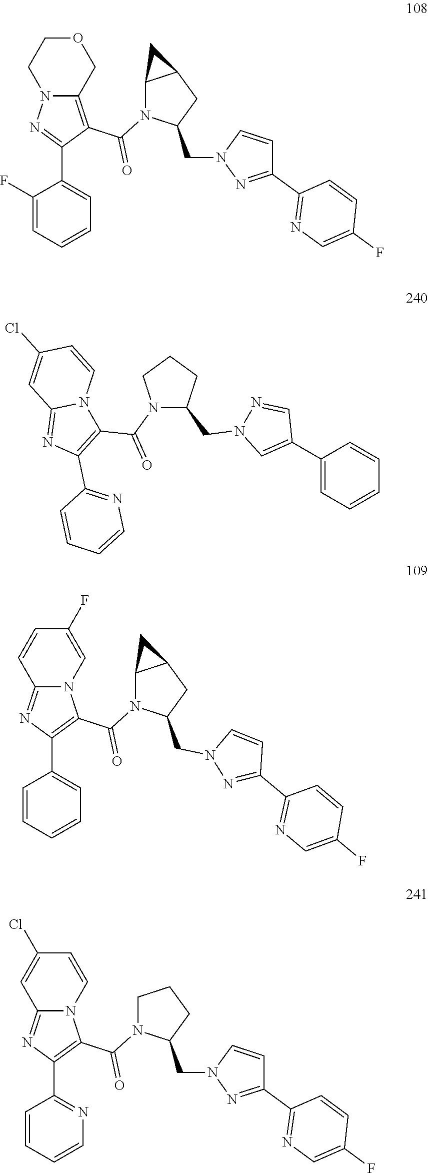 [SMILES: O=C(c1c(-c2ccccc2F)nn2c1COCC2)N1[C@H](Cn2ccc(-c3ccc(F)cn3)n2)C[C@@H]2C[C@@H]21 ; O=C(c1c(-c2ccccn2)nc2cc(Cl)ccn12)N1CCC[C@H]1Cn1cc(-c2ccccc2)cn1 ; O=C(c1c(-c2ccccc2)nc2ccc(F)cn12)N1[C@H](Cn2ccc(-c3ccc(F)cn3)n2)C[C@@H]2C[C@@H]21 ; O=C(c1c(-c2ccccn2)nc2cc(Cl)ccn12)N1CCC[C@H]1Cn1ccc(-c2ccc(F)cn2)n1] 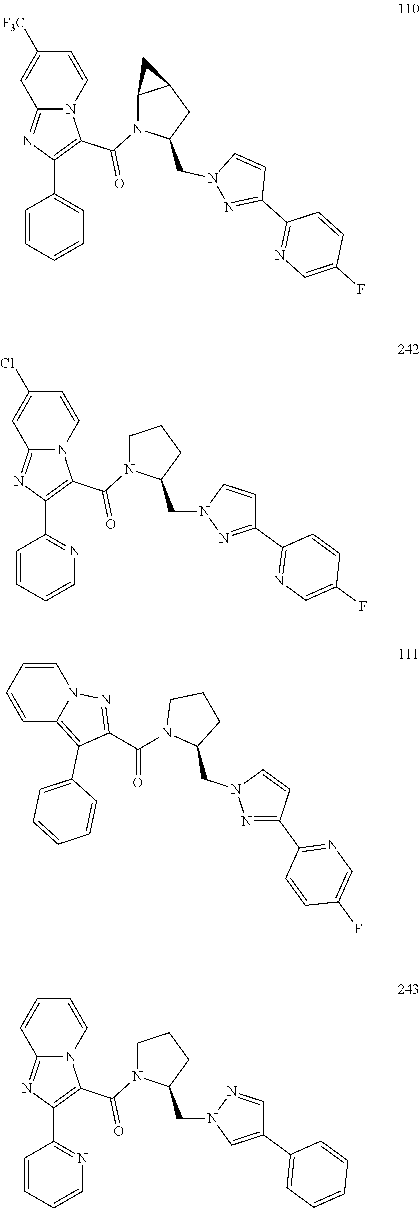 [SMILES: O=C(c1c(-c2ccccc2)nc2cc(C(F)(F)F)ccn12)N1[C@H](Cn2ccc(-c3ccc(F)cn3)n2)C[C@@H]2C[C@@H]21 ; O=C(c1c(-c2ccccn2)nc2cc(Cl)ccn12)N1CCC[C@H]1Cn1ccc(-c2ccc(F)cn2)n1 ; O=C(c1nn2ccccc2c1-c1ccccc1)N1CCC[C@H]1Cn1ccc(-c2ccc(F)cn2)n1 ; O=C(c1c(-c2ccccn2)nc2ccccn12)N1CCC[C@H]1Cn1cc(-c2ccccc2)cn1] 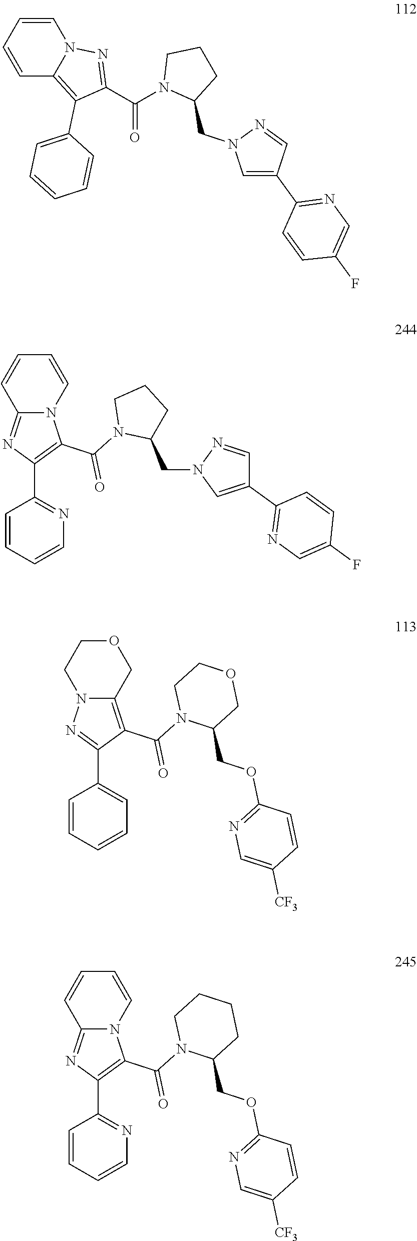 [SMILES: O=C(c1nn2ccccc2c1-c1ccccc1)N1CCC[C@H]1Cn1cc(-c2ccc(F)cn2)cn1 ; O=C(c1c(-c2ccccn2)nc2ccccn12)N1CCC[C@H]1Cn1cc(-c2ccc(F)cn2)cn1 ; O=C(c1c(-c2ccccc2)nn2c1COCC2)N1CCOC[C@H]1COc1ccc(C(F)(F)F)cn1 ; O=C(c1c(-c2ccccn2)nc2ccccn12)N1CCCC[C@H]1COc1ccc(C(F)(F)F)cn1] 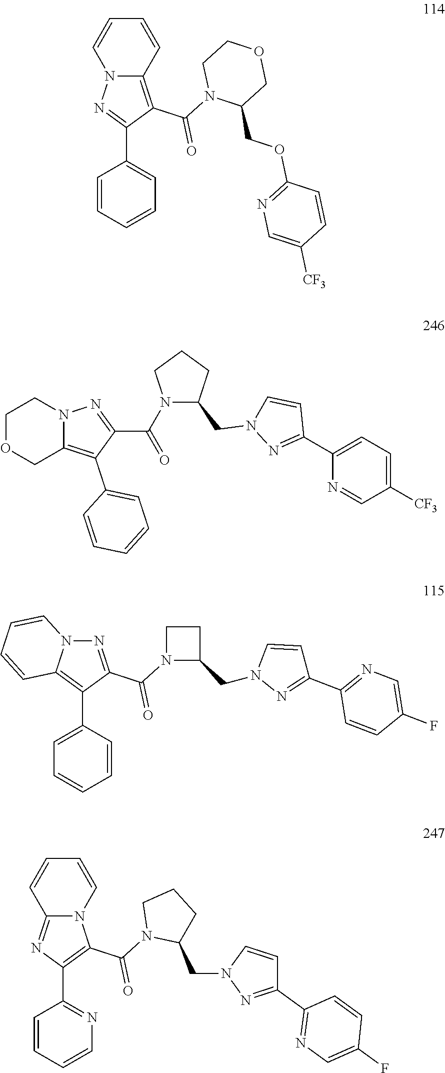 [SMILES: O=C(c1c(-c2ccccc2)nn2ccccc12)N1CCOC[C@H]1COc1ccc(C(F)(F)F)cn1 ; O=C(c1nn2c(c1-c1ccccc1)COCC2)N1CCC[C@H]1Cn1ccc(-c2ccc(C(F)(F)F)cn2)n1 ; O=C(c1nn2ccccc2c1-c1ccccc1)N1CC[C@H]1Cn1ccc(-c2ccc(F)cn2)n1 ; O=C(c1c(-c2ccccn2)nc2ccccn12)N1CCC[C@H]1Cn1ccc(-c2ccc(F)cn2)n1] 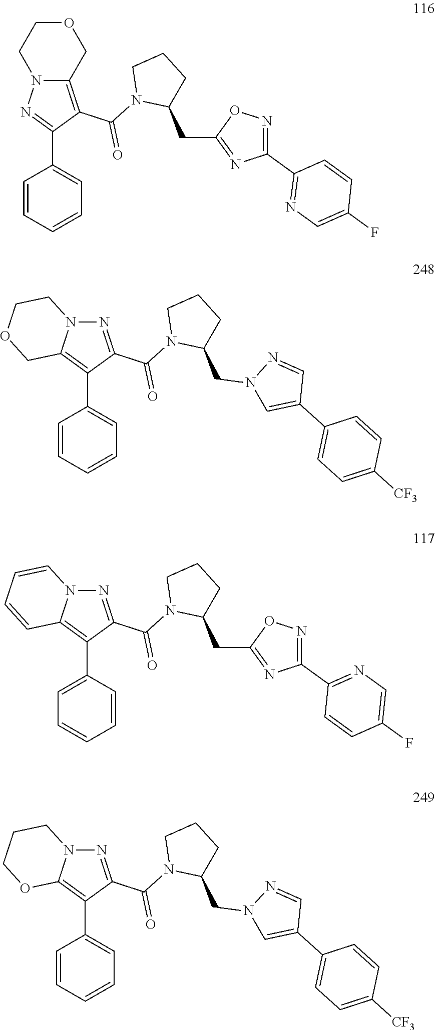 [SMILES: O=C(c1c(-c2ccccc2)nn2c1COCC2)N1CCC[C@H]1Cc1nc(-c2ccc(F)cn2)no1 ; O=C(c1nn2c(c1-c1ccccc1)COCC2)N1CCC[C@H]1Cn1cc(-c2ccc(C(F)(F)F)cc2)cn1 ; O=C(c1nn2ccccc2c1-c1ccccc1)N1CCC[C@H]1Cc1nc(-c2ccc(F)cn2)no1 ; O=C(c1nn2c(c1-c1ccccc1)OCCC2)N1CCC[C@H]1Cn1cc(-c2ccc(C(F)(F)F)cc2)cn1] 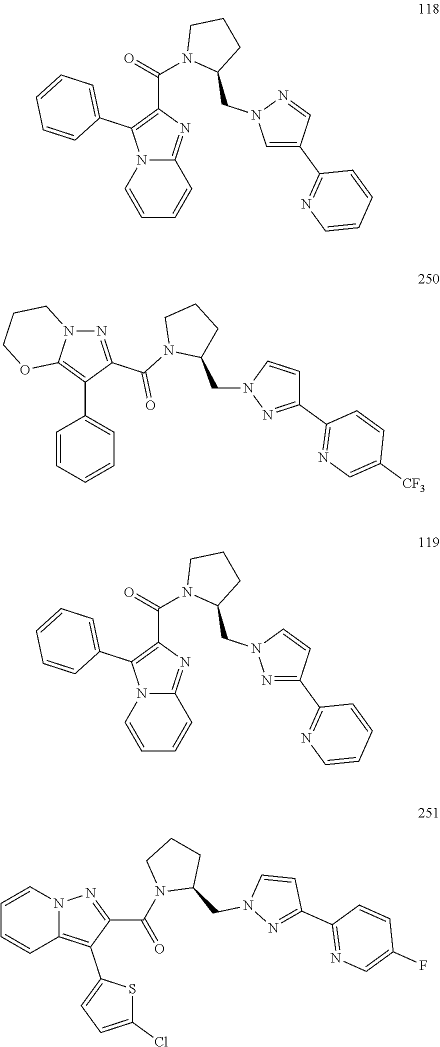 [SMILES: O=C(c1nc2ccccn2c1-c1ccccc1)N1CCC[C@H]1Cn1cc(-c2ccccn2)cn1 ; O=C(c1nn2c(c1-c1ccccc1)OCCC2)N1CCC[C@H]1Cn1ccc(-c2ccc(C(F)(F)F)cn2)n1 ; O=C(c1nc2ccccn2c1-c1ccccc1)N1CCC[C@H]1Cn1ccc(-c2ccccn2)n1 ; O=C(c1nn2ccccc2c1-c1ccc(Cl)s1)N1CCC[C@H]1Cn1ccc(-c2ccc(F)cn2)n1] 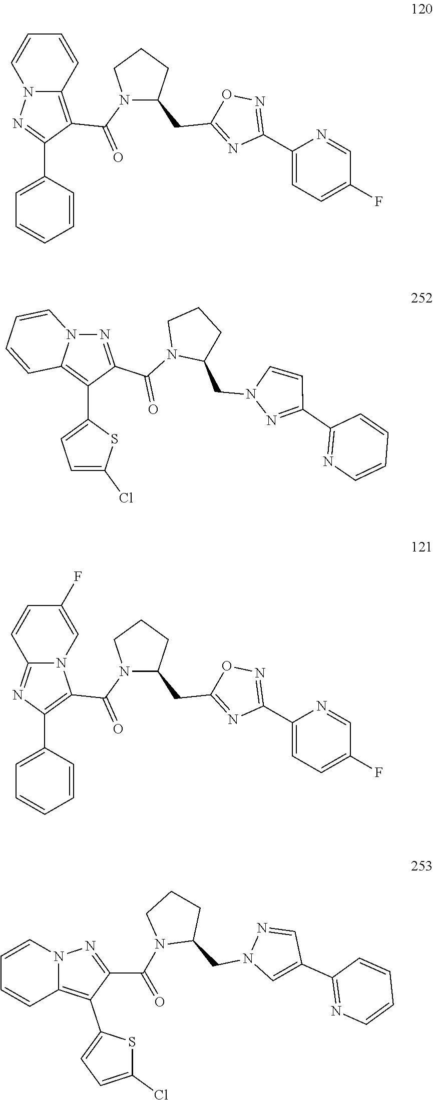 [SMILES: O=C(c1c(-c2ccccc2)nn2ccccc12)N1CCC[C@H]1Cc1nc(-c2ccc(F)cn2)no1 ; O=C(c1nn2ccccc2c1-c1ccc(Cl)s1)N1CCC[C@H]1Cn1ccc(-c2ccccn2)n1 ; O=C(c1c(-c2ccccc2)nc2ccc(F)cn12)N1CCC[C@H]1Cc1nc(-c2ccc(F)cn2)no1 ; O=C(c1nn2ccccc2c1-c1ccc(Cl)s1)N1CCC[C@H]1Cn1cc(-c2ccccn2)cn1] 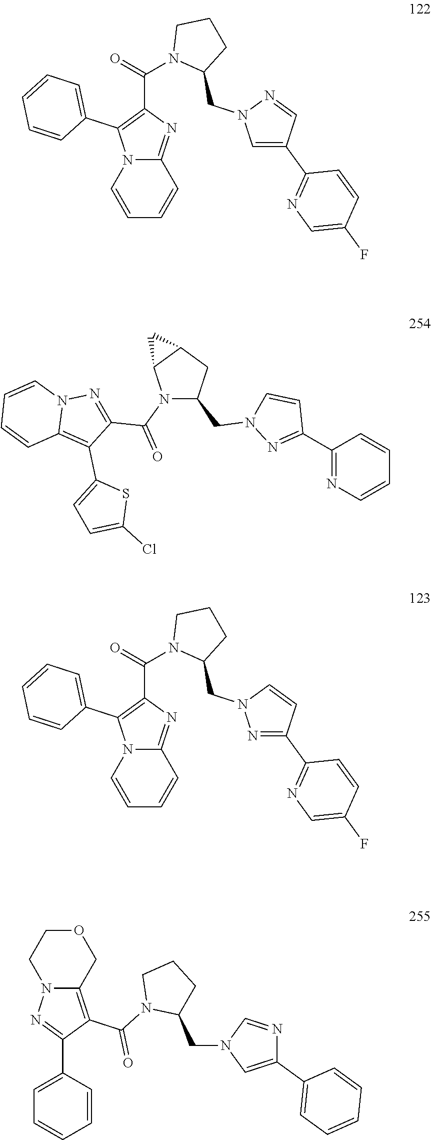 [SMILES: O=C(c1nc2ccccn2c1-c1ccccc1)N1CCC[C@H]1Cn1cc(-c2ccc(F)cn2)cn1 ; O=C(c1nn2ccccc2c1-c1ccc(Cl)s1)N1[C@H](Cn2ccc(-c3ccccn3)n2)C[C@H]2C[C@H]21 ; O=C(c1nc2ccccn2c1-c1ccccc1)N1CCC[C@H]1Cn1ccc(-c2ccc(F)cn2)n1 ; O=C(c1c(-c2ccccc2)nn2c1COCC2)N1CCC[C@H]1Cn1cnc(-c2ccccc2)c1] 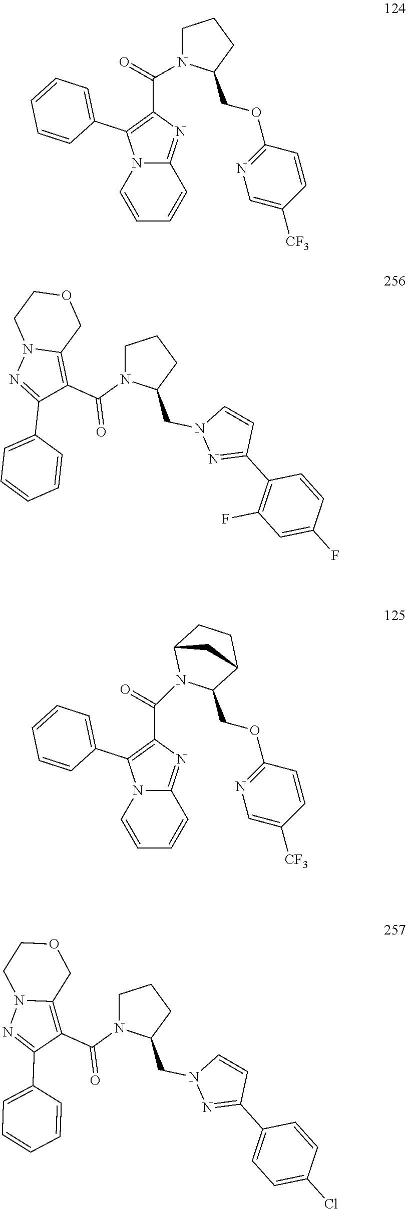 [SMILES: O=C(c1nc2ccccn2c1-c1ccccc1)N1CCC[C@H]1COc1ccc(C(F)(F)F)cn1 ; O=C(c1c(-c2ccccc2)nn2c1COCC2)N1CCC[C@H]1Cn1ccc(-c2ccc(F)cc2F)n1 ; O=C(c1nc2ccccn2c1-c1ccccc1)N1[C@@H]2CC[C@@H](C2)[C@H]1COc1ccc(C(F)(F)F)cn1 ; O=C(c1c(-c2ccccc2)nn2c1COCC2)N1CCC[C@H]1Cn1ccc(-c2ccc(Cl)cc2)n1] 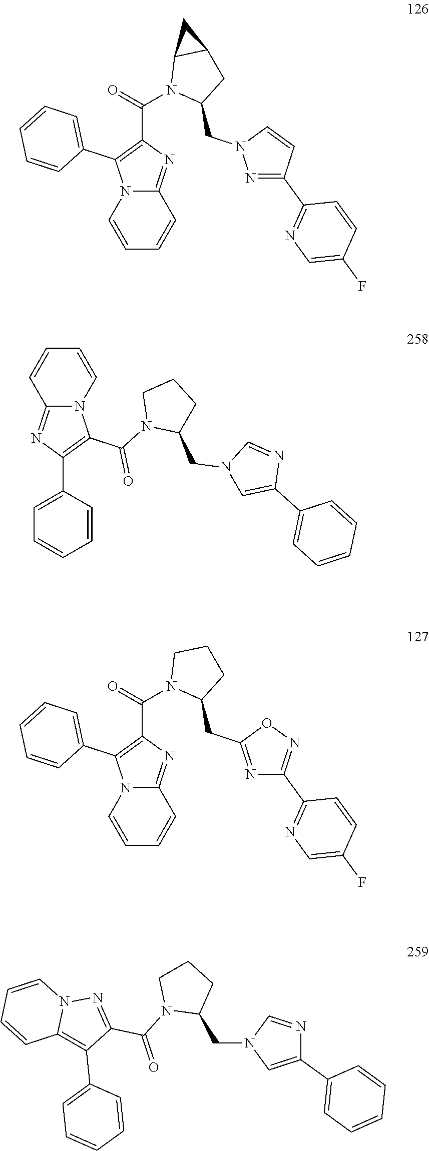 [SMILES: O=C(c1nc2ccccn2c1-c1ccccc1)N1[C@H](Cn2ccc(-c3ccc(F)cn3)n2)C[C@@H]2C[C@@H]21 ; O=C(c1c(-c2ccccc2)nc2ccccn12)N1CCC[C@H]1Cn1cnc(-c2ccccc2)c1 ; O=C(c1nc2ccccn2c1-c1ccccc1)N1CCC[C@H]1Cc1nc(-c2ccc(F)cn2)no1 ; O=C(c1nn2ccccc2c1-c1ccccc1)N1CCC[C@H]1Cn1cnc(-c2ccccc2)c1] 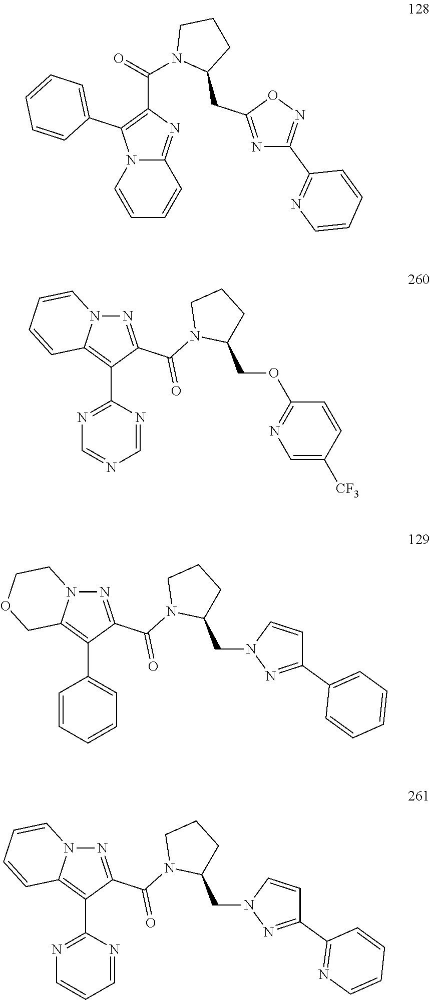 [SMILES: O=C(c1nc2ccccn2c1-c1ccccc1)N1CCC[C@H]1Cc1nc(-c2ccccn2)no1 ; O=C(c1nn2ccccc2c1-c1ncncn1)N1CCC[C@H]1COc1ccc(C(F)(F)F)cn1 ; O=C(c1nn2c(c1-c1ccccc1)COCC2)N1CCC[C@H]1Cn1ccc(-c2ccccc2)n1 ; O=C(c1nn2ccccc2c1-c1ncccn1)N1CCC[C@H]1Cn1ccc(-c2ccccn2)n1] 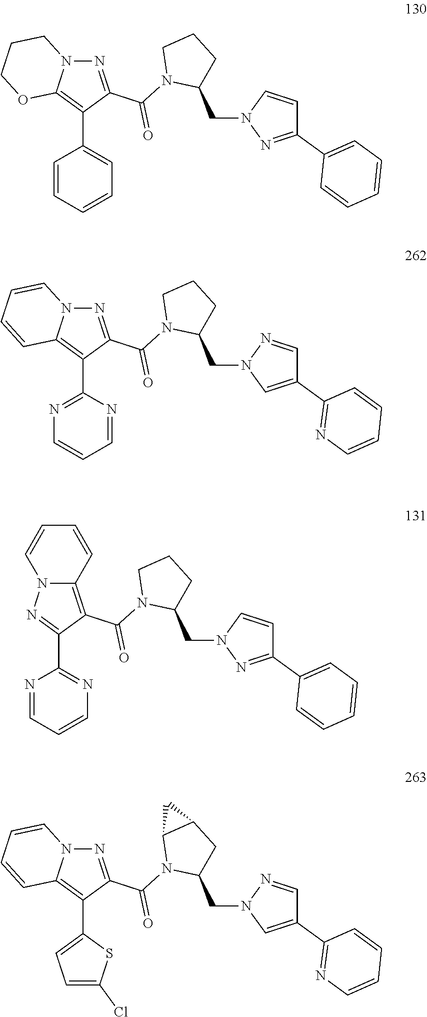 [SMILES: O=C(c1nn2c(c1-c1ccccc1)OCCC2)N1CCC[C@H]1Cn1ccc(-c2ccccc2)n1 ; O=C(c1nn2ccccc2c1-c1ncccn1)N1CCC[C@H]1Cn1cc(-c2ccccn2)cn1 ; O=C(c1c(-c2ncccn2)nn2ccccc12)N1CCC[C@H]1Cn1ccc(-c2ccccc2)n1 ; O=C(c1nn2ccccc2c1-c1ccc(Cl)s1)N1[C@H](Cn2cc(-c3ccccn3)cn2)C[C@H]2C[C@H]21]
or, a pharmaceutically acceptable salt, hydrate, solvate, polymorph, isomer, or combination thereof.

7. A compound of claim 6 selected from the group consisting of the compounds of Examples 4, 6, 7, 8, 10, 12, 13, 20, 22, 24, 25-29, 34, 40, 42-50, 53-64, 66-69, 73, 75, 78, 80, 89, 90, 92, 94, 95, 97, 107, 111, 112, 117-119, 122-142, 147-151, 156, 158, 171-183, 185-198, 201-203, 205, 207, 208, 210-214, 223, and 224; or, a pharmaceutically acceptable salt, hydrate, solvate, polymorph, isomer, or combination thereof.

8. A compound of claim 6 or 7 selected from the group consisting of the compounds of Examples 53, 55, 66, 95, 112, 118, 119, 122, 123, 124, 129, 130, 131, 134, 135, 138, 139, 140, 141, 142, 147, 148, 156, 171, 172, 173, 174, 175, 176, 177, 178, 179, 180, 181, 182, 191, 195, 203, 205, 211, 223, and 224; or, a pharmaceutically acceptable salt, hydrate, solvate, polymorph, isomer, or combination thereof.

9. A compound of any preceding claim in which the compound is unlabeled or isotopically.

10. A pharmaceutical composition comprising a compound according to any preceding claim, or a pharmaceutically acceptable salt, hydrate, solvate, polymorph, isomer, or combination thereof; and at least one pharmaceutically acceptable carrier, adjuvant and/or vehicle.

11. The pharmaceutical composition of claim 8 comprising a therapeutically effective amount of the compound, pharmaceutically acceptable salt, hydrate, solvate, polymorph, isomer, and/or combination thereof.

12. The pharmaceutical composition according to claim 10 or 11, wherein said composition further comprises at least one second therapeutic agent.

13. A method for antagonizing and/or modulating at least one orexin receptor and/or at least one kappa-opioid receptor in a cell, comprising the step of exposing the cell to a compound and/or composition according to any preceding claim, optionally wherein said method is in vitro.

14. A method for modulating at least one orexin receptor and/or at least one kappa-opioid receptor in a subject in need thereof, comprising the step of administering a compound and/or composition according to any preceding claim.

15. A method of treating a condition selected from the group consisting of substance addiction, substance dependence, panic, anxiety, depression, posttraumatic stress disorders (PTSD), neurodegeneration, autism, schizophrenia, pain, Alzheimer diseases (AD), and a central nervous system (CNS) disorder in a subject in need thereof, comprising the step of administering a compound and/or composition according to any preceding claim.

16. The method of claim 15 wherein the substance corresponding to the substance addiction or substance dependence is selected from the group consisting of one or more opioids, optionally heroin, morphine, oxycodone, fentanyl, and hydrocodone; one or more stimulants optionally selected from the group consisting of amphetamine, cocaine, crack cocaine, and methamphetamine; one or more sedatives and/or tranquilizers, benzodiazepine, and barbituates.

17. The method of any one of claims 13-16 wherein the compound antagonize at least one orexin receptor and/or antagonize or modulate at least one kappa-opioid receptor.

18. A method for manufacturing a compound or composition of any preceding claim using at least one applicable combination of acid intermediates, amine intermediates, and methods of presented in Table 2.
